(19) United States
(12) Patent Application Publication
(10) Pub. No.: US 2025/0195475 A1
(43) Pub. Date: Jun. 19, 2025

(54) CDK2 INHIBITOR AND PREPARATION METHOD AND USE THEREOF

(71) Applicants: INVENTISBIO CO., LTD., Pudong, Shanghai (CN); INVENTISBIO LLC, Short Hills, NJ (US)

(72) Inventors: Yanxin YU, Pudong Shanghai (CN); Younong YU, Florham Park, NJ (US); Xianhai HUANG, Florham Park, NJ (US); Hong YANG, Pudong, Shanghai (CN); Rongfeng LIU, Pudong, Shanghai (CN); Jifang WENG, Pudong, Shanghai (CN); Yaolin WANG, Pudong, Shanghai (CN); Xing DAI, Pudong, Shanghai (CN)

(21) Appl. No.: 18/834,118

(22) PCT Filed: Jan. 27, 2022
(86) PCT No.: PCT/CN2022/074188

§ 371 Date: Jul. 29, 2024

(51) Int. Cl. A61K 31/4155; A61K 31/4178; A61K 31/4184; A61K 31/422; A61K 31/454; A61K 31/506; A61P 35/00; C07D 231/40; C07D 401/12; C07D 403/04; C07D 403/12; C07D 403/14; C07D 405/12; C07D 405/14; C07D 413/14

(52) U.S. Cl. CPC A61K 31/4155; A61K 31/4178; A61K 31/4184; A61K 31/422; A61K 31/454; A61K 31/506; A61P 35/00; C07D 231/40; C07D 401/12; C07D 403/04; C07D 403/12; C07D 403/14; C07D 405/12; C07D 405/14; C07D 413/14

(57) ABSTRACT

The present invention discloses a compound represented by formula 1a or formula 1b, an optical isomer, prodrug or pharmaceutically acceptable salt thereof, a pharmaceutical composition thereof, and their use in preparation of a CDK2 inhibitor

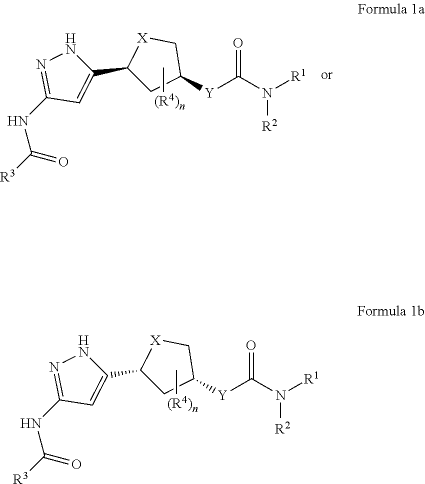

### TECHNICAL FIELD

The present invention belongs to the field of medicine, specifically relates to a compound represented by formula 1a or formula 1b, an optical isomer and pharmaceutically acceptable salt thereof, as well as the preparation method thereof, and also relates to a composition containing the compound represented by formula 1a or formula 1b, an optical isomer and pharmaceutically acceptable salt thereof, and medical use of such compound or composition as a CDK2 inhibitor.

### BACKGROUND ART

Cyclin-dependent kinase 2 (CDK2) belongs to the family of serine/threonine kinases, and its molecule activity is regulated by a fine regulatory network involving a variety of cytokines, phosphorylation/dephosphorylation modification, ubiquitin-mediated proteolysis pathway, cyclin-dependent kinase inhibitors, and so on. The abnormality in its activity regulation will lead to cell proliferation disorder and even tumorigenesis.

Many different cyclins (Cyclin A1, A2, E1, E2, etc.) are CDK2 regulatory cyclins, and they are often overexpressed in cancer. The amplification or over-expression of Cyclin E (Cyclin E1, E2) is associated with poor prognosis in breast cancer and the like. Cyclin E-CDK2 complex plays an important role in cell cycle G1/S transition, histone biosynthesis and centrosome replication.

So far, a variety of CDK4/6 inhibitors have been marketed, offering high clinical benefits to patients with advanced HR+ breast cancer. CDK4/6 inhibitors act through the “Cyclin-CDK-Rb-E2F-cell cycle-related genes” axis. Rb is a key target for CDK4/6 inhibitors to inhibit tumor cell growth. Cyclin E-CDK2 participates in and promotes cell G1/S transition through phosphorylation of Rb. The overexpression of CCNE1 (Cyclin E1) and CDK2 may lead to over-activation of Cyclin E1-CDK2 complex and phosphorylation of Rb, thereby promoting tumor cell cycle progression. The up-regulation of CCNE2 (Cyclin E2) expression and gene amplification in breast cancer cells leads to enhanced signal of cell cycle progression, which is one of the mechanisms of CDK4/6 inhibitor resistance in breast cancer. Therefore, the primary or secondary drug resistance of CDK4/6 inhibitors brings new challenges for tumor treatment.

It has been proved that harmful cytotoxicity in many failed clinical drug candidates may come from the inhibition of CDK1, which has been shown to play a central role in G2/M progression of the cell cycle. In addition, selective CDK2 inhibitors can reduce the risk of certain hematological toxicity.

Therefore, there is an urgent need to find a selective CDK2 inhibitor that is highly safe and effective for the treatment of tumors with CCNE1 or CCNE2 high expression or gene amplification.

### SUMMARY OF THE INVENTION

### Technical Problem

In view of the problems in the prior art, one of the technical objectives of the present invention is to provide a compound with a novel structure, an optical isomer and pharmaceutically acceptable salt thereof;

The second technical objective of the present invention is to provide a method for preparing the compound, the optical isomer and pharmaceutically acceptable salt thereof;

The third technical objective of the present invention is to provide a pharmaceutical composition comprising such compound, the optical isomer and pharmaceutically acceptable salt thereof;

The fourth technical objective of the present invention is to provide medical use of such compound or pharmaceutical composition as a CDK2 inhibitor.

The fifth technical objective of the present invention is to provide the use of such compound or pharmaceutical composition as a drug for treating an abnormal cell growth disease.

The sixth technical objective of the present invention is to provide a method for treating an abnormal cell growth disease, comprising administering an effective amount of the compound, an optical isomer, prodrug or pharmaceutically acceptable salt thereof or said pharmaceutical composition according to the present invention, to a subject in need thereof.

In one embodiment, the present invention provides use of the compound, the optical isomer, prodrug or pharmaceutically acceptable salt thereof in preparation of a drug for treatment of an abnormal cell growth disease.

In one embodiment, the present invention provides a method for treating an abnormal cell growth disease, comprising administering an effective amount of the compound, the optical isomer, pharmaceutically acceptable salt thereof or the pharmaceutical composition according to the present invention, to a subject in need thereof.

In one embodiment, the abnormal cell growth disease is cancer.

In one embodiment, the cancer is selected from breast cancer, ovarian cancer, bladder cancer, uterine cancer, prostate cancer, lung cancer, esophageal cancer, head and neck cancer, colorectal cancer, kidney cancer (including renal cell carcinoma), liver cancer (including hepatocellular carcinoma), pancreatic cancer, gastric cancer or thyroid cancer.

In one embodiment, the lung cancer is selected from non-small cell lung cancer, small cell lung cancer, squamous cell carcinoma or adenocarcinoma.

In one embodiment, the present invention provides a compound represented by formula 1a or formula 1b, or an optical isomer or pharmaceutically acceptable salt thereof:

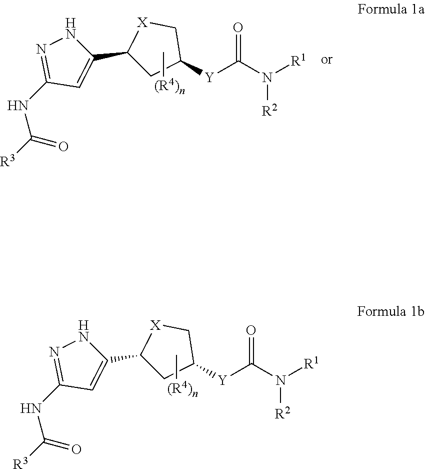

wherein,
X is selected from C, O or S, and X bonds with 1 or 2 hydrogen atoms or without hydrogen atoms attached according to the bonding valence;
Y is selected from C, O or N, preferably O or N, and Y bonds with 1 or 2 hydrogen atoms or without hydrogen atoms attached according to the bonding valence; or Y together with the

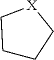

to which Y is bonded forms an optionally substituted 6 to 8 membered aliphatic fused ring, or a optionally substituted 6 to 8 membered aliphatic hetero fused ring containing 1 to 3 heteroatoms selected from N, O, and S;
R¹ and R² are independently selected from hydrogen, optionally substituted C1 to C8 alkyl, optionally substituted C2 to C8 alkoxy, optionally substituted C3 to C15 cycloalkyl, optionally substituted 4 to 15 membered heterocycloalkyl containing 1 to 3 heteroatoms selected from N, O, and S, optionally substituted 4 to 15 membered aliphatic fused ring, optionally substituted 5 to 15 membered aliphatic spiro ring, optionally substituted 5 to 15 membered aliphatic bridged ring, optionally substituted 5 to 15 membered aliphatic hetero fused ring containing 1 to 3 heteroatoms selected from N, O, and S, optionally substituted 6 to 15 membered aliphatic hetero spiro ring containing 1 to 3 heteroatoms selected from N, O, and S, and optionally substituted 5 to 15 membered aliphatic hetero bridged ring containing 1 to 3 heteroatoms selected from N, O, and S heteroatom; or
R¹ and R² together to form an optionally substituted 4 to 15 membered heterocycloalkyl containing 1 to 3 heteroatoms selected from N, O, and S, an optionally substituted 5 to 15 membered aliphatic hetero fused ring containing 1 to 3 heteroatoms selected from N, O, and S in addition to the N atom to which R¹ and R² are bonded, an optionally substituted 6 to 15 membered aliphatic hetero spiro ring containing 1 to 3 heteroatoms selected from N, O, and S in addition to the N atom to which R¹ and R² are bonded, or an optionally substituted 5 to 15 membered aliphatic hetero bridged ring containing 1 to 3 heteroatoms selected from N, O, and S in addition to the N atom to which R¹ and R² are bonded; or
one of R¹ and R², together with the N atom to which R¹ and R² are bonded, the carbonyl group and Y, forms an optionally substituted 5 to 10 membered heterocycloalkyl containing 1 to 3 heteroatoms selected from N, O, and S;
wherein the “substituted” in the above definition of R¹ and R² means that the group comprises 1 to 3 substituents selected from C1 to C6 alkyl, C1 to C6 haloalkyl, C1 to C6 alkoxy, C1 to C6 haloalkoxy, C2 to C6 alkenyl, C2 to C6 haloalkenyl, C2 to C6 alkynyl, C2 to C6 haloalkynyl, C3 to C6 cycloalkyl, C3 to C6 halocycloalkyl, C4 to C6 heterocycloalkyl, C4 to C6 haloheterocycloalkyl, hydroxyl, amino, sulfone group, cyano, and halogen atom;
R³ is selected from hydrogen, optionally substituted C1 to C15 alkyl, optionally substituted C2 to C15 alkenyl, optionally substituted C2 to C15 alkynyl, optionally substituted C2 to C15 alkoxy, optionally substituted C3 to C15 cycloalkyl, optionally substituted 4 to 15 membered heterocycloalkyl containing 1 to 3 heteroatoms selected from N, O, and S, optionally substituted C6 to C14 aryl, optionally substituted 5 to 15 membered heteroaryl containing 1 to 3 heteroatoms selected from N, O, and S, optionally substituted 4 to 17 membered aliphatic fused ring, optionally substituted 5 to 17 membered aliphatic bridged ring, optionally substituted 5 to 15 membered aliphatic spiro ring, optionally substituted 4 to 17 membered aliphatic hetero fused ring containing 1 to 3 heteroatoms selected from N, O, and S, optionally substituted 5 to 17 membered aliphatic hetero bridged ring containing 1 to 3 heteroatoms selected from N, O and S heteroatoms, optionally substituted 5 to 17 membered aromatic fused ring, optionally substituted 5 to 17 membered aromatic hetero fused ring containing 1 to 3 heteroatoms selected from N, O, and S, and optionally substituted 4 to 17 membered aliphatic hetero spiro ring containing 1 to 3 heteroatoms selected from N, O, and S, C8-C14 cycloalkyl fused heteroaryl and C8-C14 heterocycloalkyl fused heteroaryl, wherein the “substituted” means that the group comprises 1 to 3 substituents selected from the following groups: optionally substituted C1 to C6 alkyl, optionally substituted C1 to C6 alkoxy, hydroxy-substituted C1 to C6 alkyl group, amino-substituted C1 to C6 alkyl group, C1 to C6 alkyl group substituted with 1, 2, or 3 halogen atoms, C1 to C6 alkoxy substituted with 1, 2, or 3 halogen atoms, optionally substituted C2 to C7 alkoxyalkyl, C2 to C7 alkoxyalkyl substituted with 1, 2, or 3 halogen atoms, optionally substituted C2 to C6 alkenyl, optionally substituted C2 to C6 alkynyl, optionally substituted C3 to C6 cycloalkyl, optionally substituted 3 to 6 membered heterocycloalkyl containing 1 to 3 heteroatoms selected from N, O, and S, optionally substituted C6 to C10 aryl, optionally substituted 5 to 10 membered heteroaryl containing 1 to 3 heteroatoms selected from N, O, and S, optionally substituted 6 to 10 membered fused heterocyclyl containing 1 to 3 heteroatoms selected from N, O, and S, optionally substituted 9 to 16 membered cycloalkyl fused aryl, optionally substituted 9 to 16 membered heterocycloalkyl fused aryl containing 1 to 3 heteroatoms selected from N, O and S, optionally substituted 8 to 15 membered cycloalkyl fused heteroaryl containing 1 to 5 heteroatoms selected from N, O and S, and optionally substituted 8 to 15 membered heterocycloalkyl fused heteroaryl containing 1 to 5 heteroatoms selected from N, O and S, optionally substituted C3 to C6 cycloalkylsulfonyl, optionally substituted C1 to C6 alkylsulfonyl, C1 to C6 haloalkyl sulfonyl, optionally substituted C1 to C6 alkylacyl, C1 to C6 haloalkylacyl, optionally substituted C6 to C15 arylacyl, optionally substituted 5 to 15 membered heteroarylacyl containing 1 to 3 heteroatoms selected from N, O, and S, cyano, and halo;
R⁴ is selected from hydrogen, hydroxyl, cyano, halogen atom, C1 to C6 alkyl, and C1 to C6 alkoxy; or R⁴ together with the

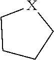

to which R⁴ is bonded forms an optionally substituted 6 to 8 membered aliphatic fused ring, or an optionally substituted 6 to 8 membered aliphatic hetero fused ring containing 1 to 3 heteroatoms selected from N, O, and S, wherein the “substituted” means that the group comprises 1 to 3 substituents selected from C1 to C6 alkyl, C1 to C6 alkoxy, C2 to C6 alkenyl, C2 to C6 alkynyl, C3 to C6 cycloalkyl, cyano, hydroxyl, halogen atom;
the subscript n is an integer from 0 to 4.

In one embodiment, the subscript n is 0, 1, 2, 3, or 4.

In one embodiment, the subscript n is 0, 1, 2, or 3.

In one embodiment, the subscript n is 0, 1, or 2.

In one embodiment, the subscript n is 0 or 1.

In one embodiment, preferably, R¹ and R² are the same or different from each other and are each independently selected from hydrogen, optionally substituted C1 to C6 alkyl, optionally substituted C2 to C6 alkoxy, optionally substituted C3 to C10 cycloalkyl, optionally substituted 4 to 10 membered heterocycloalkyl containing 1 to 3 heteroatoms selected from N, O, and S, optionally substituted 4 to 13 membered aliphatic fused ring, optionally substituted 5 to 13 membered aliphatic spiro ring, optionally substituted 5 to 13 membered aliphatic bridged ring, optionally substituted 5 to 13 membered aliphatic hetero fused ring containing 1 to 3 heteroatoms selected from N, O, and S, optionally substituted 6 to 13 membered aliphatic hetero spiro ring containing 1 to 3 heteroatoms selected from N, O, and S, and optionally substituted 5 to 13 membered aliphatic hetero bridged ring containing 1 to 3 heteroatoms selected from N, O, and S; or
R¹ and R² together to form an optionally substituted 4 to 10 membered heterocycloalkyl containing 1 to 3 heteroatoms selected from N, O, and S, an optionally substituted 5 to 10 membered aliphatic hetero fused ring containing 1 to 3 heteroatoms selected from N, O, and S in addition to the N atom to which R¹ and R² are bonded, an optionally substituted 6 to 13 membered aliphatic hetero spiro ring containing 1 to 3 heteroatoms selected from N, O, and S in addition to the N atom to which R¹ and R² are bonded, or an optionally substituted 5 to 13 membered aliphatic hetero bridged ring containing 1 to 3 heteroatoms selected from N, O, and S in addition to the N atom to which R¹ and R² are bonded; or
one of R¹ and R², together with the N atom to which they are bonded, the carbonyl group and Y, forms an optionally substituted 5 to 8 membered heterocycloalkyl containing 1 to 3 heteroatoms selected from N, O, and S.

In one embodiment, more preferably, R¹ and R² are independently selected from hydrogen, optionally substituted C1 to C4 alkyl, optionally substituted C2 to C4 alkoxy, optionally substituted C3 to C8 cycloalkyl, optionally substituted 4 to 8 membered heterocycloalkyl containing 1 to 3 heteroatoms selected from N, O, and S, optionally substituted 4 to 10 membered aliphatic fused ring, optionally substituted 5 to 10 membered aliphatic spiro ring, optionally substituted 5 to 10 membered aliphatic bridged ring, optionally substituted 5 to 10 membered aliphatic hetero fused ring containing 1 to 3 heteroatoms selected from N, O, and S, optionally substituted 6 to 10 membered aliphatic hetero spiro ring containing 1 to 3 heteroatoms selected from N, O, and S, and optionally substituted 5 to 10 membered aliphatic hetero bridged ring containing 1 to 3 heteroatoms selected from N, O, and S; or
R¹ and R² together to form an optionally substituted 4 to 8 membered heterocycloalkyl containing 1 to 3 heteroatoms selected from N, O, and S, an optionally substituted 4 to 10 membered aliphatic hetero fused ring containing 1 to 3 heteroatoms selected from N, O, and S in addition to the N atom to which R¹ and R² are bonded, an optionally substituted 6 to 10 membered aliphatic hetero spiro ring containing 1 to 3 heteroatoms selected from N, O, and S in addition to the N atom to which R¹ and R² are bonded, or an optionally substituted 5 to 10 membered aliphatic hetero bridged ring containing 1 to 3 heteroatoms selected from N, O, and S in addition to the N atom to which R¹ and R² are bonded; or
one of R¹ and R², together with the N atom to which they are bonded, the carbonyl group and Y forms an optionally substituted 5 to 6 membered heterocycloalkyl containing 1 to 3 heteroatoms selected from N, O, and S.

In one embodiment, more preferably, R¹ and R² are the same or different from each other and are each independently selected from hydrogen, methyl, ethyl, n-propyl, isopropyl, 1,1-difluoroisopropyl, n-butyl, isobutyl, tert-butyl, ethoxy, n-propoxy, isopropoxy, n-butoxy, isobutoxy, tert-butoxy, cyclopropyl, cyclobutyl, cyclopentyl, cyclohexyl, methylcyclopropyl, ethylcyclopropyl, n-propylcyclopropyl, isopropylcyclopropyl, n-butylcyclopropyl, isobutylcyclopropyl, methylcyclobutyl, ethylcyclobutyl, n-propylcyclobutyl, isopropylcyclobutyl, n-butylcyclobutyl, isobutylcyclobutyl, methylcyclopentyl, ethylcyclopentyl, n-propylcyclopentyl, isopropylcyclopentyl n-butylcyclopentyl, isobutylcyclopentyl, oxetanyl, tetrahydrofuranyl, tetrahydropyranyl, azetidinyl, pyrrolidinyl, piperidinyl, methyloxetanyl, ethyloxetanyl, n-propyloxetanyl, isopropyloxetanyl, n-butyloxetanyl, isobutyloxetanyl, methyl tetrahydrofuranyl, ethyl tetrahydrofuranyl, n-propyl tetrahydrofuranyl, isopropyl tetrahydrofuranyl, n-butyl tetrahydrofuranyl, isobutyl tetrahydrofuranyl, methylazetidinyl, ethylazetidinyl, n-propylazetidinyl, isopropylazetidinyl, n-butylazetidinyl, isobutylazetidinyl, methylpyrrolidinyl, ethylpyrrolidinyl, n-propylpyrrolidinyl, isopropylpyrrolidinyl, n-butylpyrrolidinyl, isobutylpyrrolidinyl,

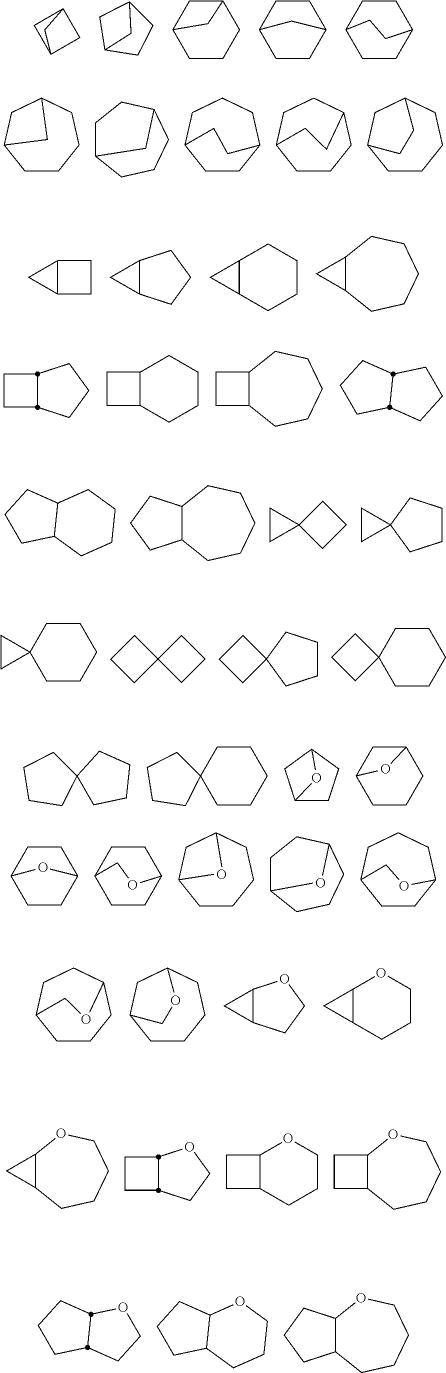

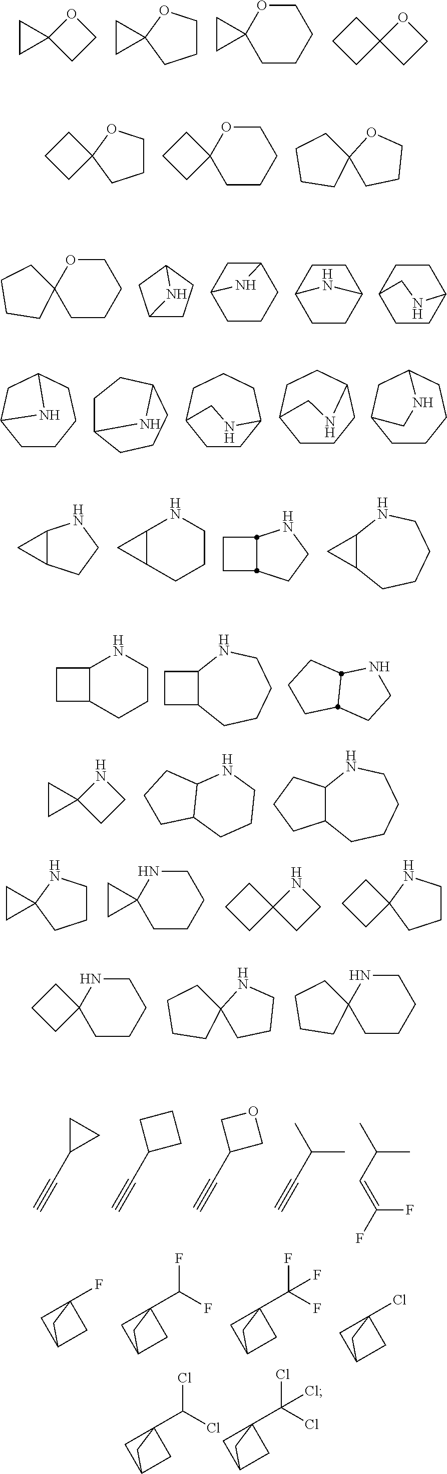

R¹ and R² together to form an optionally substituted 4 to 8 membered heterocycloalkyl containing 1 to 3 heteroatoms selected from N, O, and S, an optionally substituted 5 to 10 membered aliphatic hetero fused ring containing 1 to 3 heteroatoms selected from N, O, and S in addition to the N atom to which R¹ and R² are bonded, an optionally substituted 6 to 10 membered aliphatic hetero spiro ring containing 1 to 3 heteroatoms selected from N, O, and S in addition to the N atom to which R¹ and R² are bonded, or an optionally substituted 5 to 10 membered aliphatic hetero bridged ring containing 1 to 3 heteroatoms selected from N, O, and S in addition to the N atom to which R¹ and R² are bonded; or
one of R¹ and R², together with the N atom to which they are bonded, the carbonyl and Y, forms an optionally substituted 5 to 6 membered heterocycloalkyl containing 1 to 3 heteroatoms selected from N, O, and S.

In one embodiment, preferably, R⁴ is selected from hydrogen, C1 to C3 alkyl (e.g. methyl, ethyl, n-propyl, isopropyl), and C1 to C3 alkoxy (e.g. methoxy, ethoxy, n-propoxy, isopropoxy), or R⁴ together with the

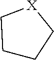

to which R⁴ is bonded forms

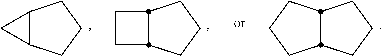

In one embodiment, the compound is a compound represented by the following formula 1-1a or formula 1-1b:

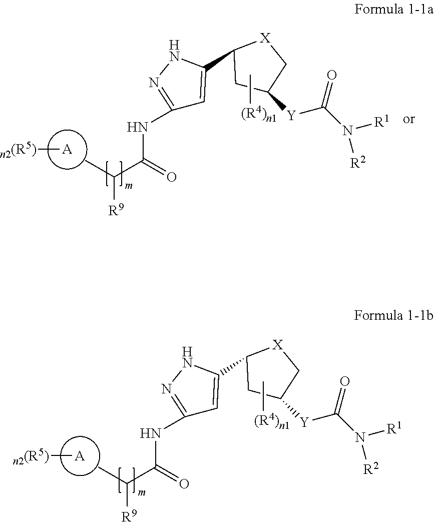

wherein,
X, Y, R¹, R², and R⁴ are defined the same as those in Formula 1a or Formula 1b, and n1 is defined the same as n in Formula 1a or Formula 1b;

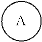

is selected from optionally substituted C3 to C15 cycloalkyl, optionally substituted 4 to 15 membered heterocycloalkyl containing 1 to 3 heteroatoms selected from N, O, and S, optionally substituted 6 to 14 membered aryl, optionally substituted 5 to 15 membered heteroaryl containing 1 to 3 heteroatoms selected from N, O, and S, optionally substituted 4 to 17 membered aliphatic fused ring, optionally substituted 5 to 17 membered aliphatic bridged ring, optionally substituted 5 to 17 membered aliphatic spiro ring, optionally substituted 4 to 17 membered aliphatic hetero fused ring containing 1 to 3 heteroatoms selected from N, O, and S, optionally substituted 5 to 17 membered aliphatic hetero bridged ring containing 1 to 3 heteroatoms selected from N, O, and S, optionally substituted 5 to 17 membered aliphatic hetero spiro ring containing 1 to 3 heteroatoms selected from N, O, and S, and optionally substituted 5 to 17 membered aromatic hetero fused ring containing 1 to 3 heteroatoms selected from N, O, and S, C8-C14 cycloalkyl fused heteroaryl and C8-C14 heterocycloalkyl fused heteroaryl;
R⁵ is selected from hydrogen, optionally substituted C1 to C6 alkyl, optionally substituted C1 to C6 alkylcarbonyl, optionally substituted C1 to C6 alkoxy, optionally substituted C1 to C6 alkoxycarbonyl, C1 to C6 alkoxy substituted with C1 to C6 alkyl, optionally substituted C2 to C6 alkenyl, optionally substituted C2 to C6 alkynyl, sulfonyl, optionally substituted C1 to C6 alkylsulfonyl, optionally substituted C3 to C6 cycloalkylsulfonyl, C1 to C6 haloalkylsulfonyl, carbonyl, cyano, halogen atom, hydroxyl, C1 to C6 alkyl acyl, C1 to C6 haloalkyl acyl, optionally substituted C6 to C14 arylacyl, optionally substituted 5 to 15 membered heteroarylacyl containing 1 to 3 heteroatoms selected from N, O, and S, optionally substituted C3 to C8 cycloalkyl, saturated or unsaturated 4 to 8 membered heterocycloalkyl containing 1 to 3 heteroatoms selected from N, O, and S, optionally substituted 6 to 10 membered aryl, optionally substituted 5 to 10 membered heteroaryl containing 1 to 3 heteroatoms selected from N, O, and S, optionally substituted 8 to 15 membered aromatic fused ring, optionally substituted 8 to 15 membered aromatic hetero fused ring containing 1 to 3 heteroatoms selected from N, O, and S, optionally substituted 9 to 16 membered cycloalkyl fused aryl, optionally substituted 9 to 16 membered heterocycloalkyl fused aryl containing 1 to 3 heteroatoms selected from N, O and S, optionally substituted 8 to 15 membered cycloalkyl fused heteroaryl containing 1 to 5 heteroatoms selected from N, O and S, and optionally substituted 8 to 15 membered heterocycloalkyl fused heteroaryl containing 1 to 5 heteroatoms selected from N, O and S, wherein the “substituted” means that the group comprises 1 to 3 substituents selected from C1 to C6 alkyl, C1 to C6 alkoxy, C1 to C6 alkyl substituted with 1, 2, or 3 halogen atoms, C1 to C6 alkoxy substituted with 1, 2, or 3 halogen atoms, C2 to C6 alkenyl, C2 to C6 alkynyl, C3 to C6 cycloalkyl, cyano, nitro, halogen atom, hydroxyl, amino, amide, hydroxylamino, C3 to C6 cycloalkyl, 3 to 6 membered heterocycloalkyl containing 1 to 3 heteroatoms selected from N, O, and S, 6 to 8 membered aryl, and 3 to 6 membered heteroaryl containing 1 to 3 heteroatoms selected from N, O, and S;
n2 is an integer from 0 to 4;
R⁹ is selected from hydrogen, C1 to C4 alkyl, C1 to C4 alkoxy, C1 to C4 haloalkyl, C1 to C4 haloalkoxy;
m is an integer from 0 to 4.

In one embodiment, n2 is 0, 1, 2, 3, or 4.

In one embodiment, n2 is 0, 1, 2, or 3.

In one embodiment, n2 is 0, 1, or 2.

In one embodiment, m is 0, 1, 2, 3, or 4.

In one embodiment, m is 0, 1, 2, or 3.

In one embodiment, m is 0, 1, or 2.

In one embodiment, preferably,

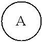

is selected from optionally substituted C3 to C10 cycloalkyl, optionally substituted 3 to 10 membered heterocycloalkyl containing 1 to 3 heteroatoms selected from N, O and S, optionally substituted 6 to 10 membered aryl, optionally substituted 5 to 10 membered heteroaryl containing 1 to 3 heteroatoms selected from N, O, and S, optionally substituted 4 to 13 membered aliphatic fused ring, optionally substituted 5 to 13 membered aliphatic bridged ring, optionally substituted 4 to 13 membered aliphatic hetero fused ring containing unsubstituted 1 to 3 heteroatoms selected from N, O, and S, optionally substituted 5 to 13 membered aliphatic hetero bridged ring containing 1 to 3 heteroatoms selected from N, O and S, optionally substituted 5 to 13 membered aromatic fused ring, and optionally substituted 4 to 13 membered aromatic hetero fused ring containing 1 to 3 heteroatoms selected from N, O, and S.

In one embodiment, more preferably,

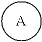

is selected from optionally substituted C3 to C8 cycloalkyl, optionally substituted 3 to 8 membered heterocycloalkyl containing 1 to 3 heteroatoms selected from N, O and S, optionally substituted 6 to 8 membered aryl, optionally substituted 5 to 8 membered heteroaryl containing 1 to 3 heteroatoms selected from N, O and S, optionally substituted 4 to 10 membered aliphatic fused ring, optionally substituted 4 to 10 membered aliphatic spiro ring, saturated or unsaturated substituted unsubstituted 5 to 10 membered aliphatic bridged ring, optionally substituted 4 to 10 membered aliphatic hetero fused ring containing 1 to 3 heteroatoms selected from N, O and S, optionally substituted 5 to 10 membered aliphatic hetero spiro ring containing 1 to 3 heteroatoms selected from N, O and S, optionally substituted 5 to 10 membered aliphatic hetero bridged ring containing 1 to 3 heteroatoms selected from N, O and S, optionally substituted 5 to 10 membered aromatic fused ring, and optionally substituted 4 to 10 membered aromatic hetero fused ring containing 1 to 3 heteroatoms selected from N, O and S.

In one embodiment, more preferably, the specific examples of

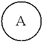

are selected from:

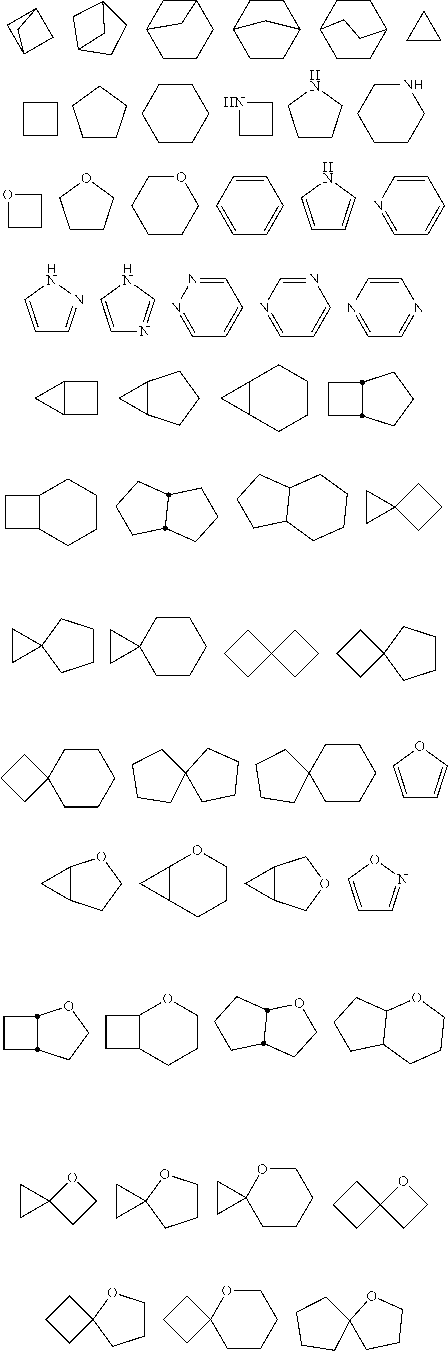

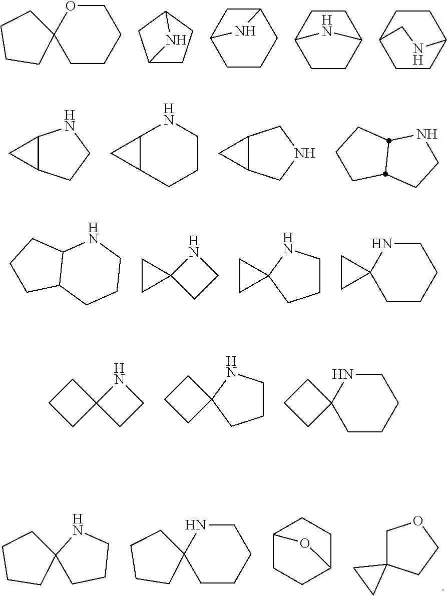

In one embodiment, preferably, R⁹ is selected from hydrogen, methyl, ethyl, n-propyl, isopropyl, n-butyl, isobutyl, tert-butyl, methoxy, ethoxy, n-propyloxy, isopropoxy, n-butoxy, isobutoxy, tert-butoxy, monofluoromethyl, difluoromethyl, trifluoromethyl, dichloromethyl, trichloromethyl, monofluoroethyl, difluoroethyl, trifluoroethyl, tetrafluoroethyl, pentafluoroethyl, dichloroethyl, trichloroethyl, tetrachloroethyl, pentachloroethyl, difluoropropyl, trifluoropropyl, tetrafluoropropyl, pentafluoropropyl, hexafluoropropyl, perfluoropropyl, monochloropropyl, dichloropropyl, trichloropropyl, tetrachloropropyl, pentachloropropyl, hexachloropropyl, perchloropropyl.

In one embodiment, the compound is a compound represented by the following formula 1-1-1a, formula 1-1-1b, formula 1-1-2a, formula 1-1-2b, formula 1-1-3a, formula 1-1-3b, formula 1-1-4a or formula 1-1-4b:

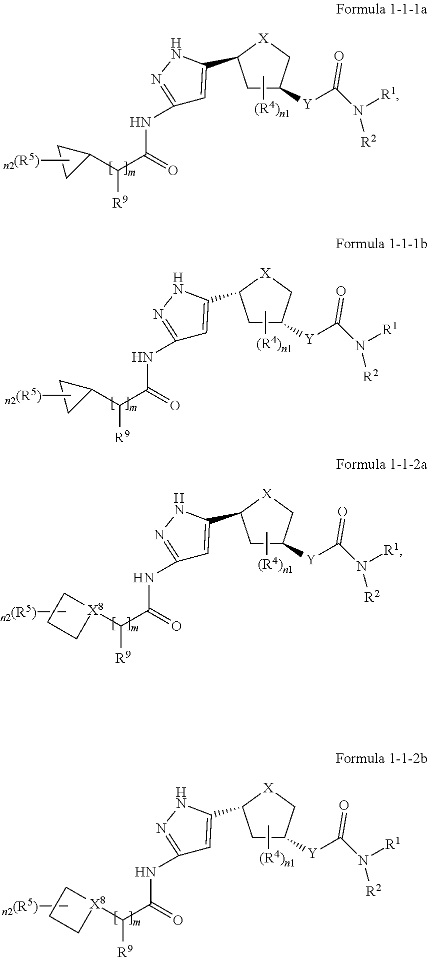

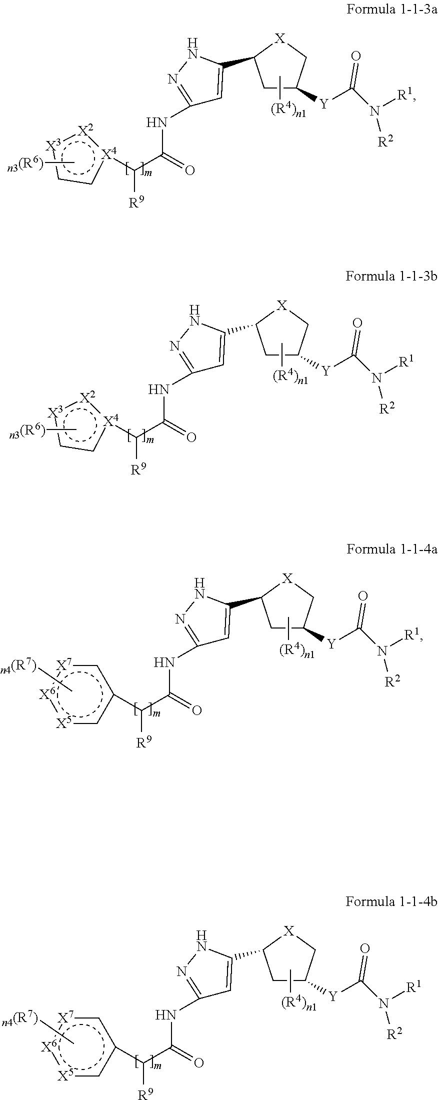

wherein,
X, Y, R¹, R², R⁴ are defined the same as those in Formula 1a or Formula 1b, n1 is defined the same as n in Formula 1a or Formula 1b;
R⁵, R⁹ and m are defined the same as those in formula 1-1a or formula 1-1b;
X² to X⁸ are each independently selected from C, O, S, and N, and X² to X⁸ each independently bond with 1 or 2 hydrogen atoms or without hydrogen atoms attached according to the bonding valence;
R⁶ is selected from hydrogen, C1 to C6 alkyl (e.g., methyl, ethyl, n-propyl, isopropyl, n-butyl, isobutyl, tert-butyl, n-pentyl, isopentyl, n-hexyl, 2-methylpentyl, 2,2-dimethylbutyl, 2,3-dimethylbutyl), C1 to C6 alkoxy (e.g. methoxy, ethoxy, n-propoxy, isopropoxy, n-butoxy, iso-butoxy, tert-butoxy, n-pentoxy, iso-pentoxy, n-hexyloxy, 2-methylpentoxy, 2,2-dimethylbutoxy, 2,3-dimethylbutoxy), C2 to C8 alkoxyalkyl (e.g., methoxymethyl, ethoxymethyl, propoxymethyl, butoxymethyl, pentoxymethyl, hexoxymethyl, heptoxymethyl, methoxyethyl, ethoxyethyl, propoxyethyl, butoxyethyl, pentoxyethyl, hexoxyethyl, methoxypropyl, ethoxypropyl, propoxypropyl, butoxypropyl, pentoxypropyl, methoxybutyl, ethoxybutyl, propoxybutyl, butoxybutyl, methoxypentyl, ethoxypentyl, propoxypentyl, methoxyhexyl, ethoxyhexyl, methoxyheptyl), C1 to C6 haloalkyl (e.g., momofluoromethyl, difluoromethyl, trifluoromethyl, dichloromethyl, trichloromethyl, monofluoroethyl, difluoroethyl, trifluoroethyl, tetrafluoroethyl, pentafluoroethyl, dichloroethyl, trichloroethyl, tetrachloroethyl, pentachloroethyl, difluoropropyl, trifluoropropyl, tetrafluoropropyl, pentafluoropropyl, hexafluoropropyl, perfluoropropyl, monochloropropyl, dichloropropyl, trichloropropyl, tetrachloropropyl, pentachloropropyl, hexachloropropyl, perchloropropyl), C1 to C6 haloalkoxy (e.g., monofluoromethoxy, difluoromethyloxy, trifluoromethoxy, dichloromethoxy, trichloromethoxy, monofluoroethoxy, difluoroethoxy, trifluoroethoxy, tetrafluoroethoxy, pentafluoroethoxy, dichloroethoxy, trichloroethoxy, tetrachloroethoxy, pentachloroethoxy, difluoropropoxy, trifluoropropoxy, tetrafluoropropoxy, pentafluoropropoxy, hexafluoropropoxy, perfluoropropoxy, monochloropropoxy, dichloropropoxy, trichloropropoxy, tetrachloropropoxy, pentachloropropoxy, hexachloropropoxy, perchloropropoxy), C1 to C6 alkylcarbonyl (e.g. methylcarbonyl, ethylcarbonyl, propylcarbonyl, butylcarbonyl, pentylcarbonyl, hexylcarbonyl), C3 to C6 cycloalkyl (e.g. cyclopropyl, cyclobutyl, cyclopentyl, cyclohexyl), C3 to C6 cycloalkylcarbonyl (e.g., cyclopropylcarbonyl, cyclobutylcarbonyl, cyclopentylcarbonyl, cyclohexylcarbonyl), 4 to 6 membered heterocycloalkyl containing 1 to 3 heteroatoms selected from N, O and S (e.g. oxetanyl, azetidinyl, thietanyl, furan, tetrahydrofuran, pyrrole, tetrahydropyrrole, oxazole, thiazole, imidazole, pyrazole, thiophene, pyridine, pyrimidine, pyridazine, pyrazine, triazine, pyran, dioxane), 4 to 6 membered heterocycloalkylcarbonyl containing 1 to 3 heteroatoms selected from N, O, and S (e.g., oxetanylcarbonyl, azetidinylcarbonyl, thietanecarbonyl, furylcarbonyl, tetrahydrofurylcarbonyl, pyrrolylcarbonyl, tetrahydropyrrolylcarbonyl, oxazolecarbonyl, thiazolylcarbonyl, imidazolylcarbonyl, pyrazolylcarbonyl, thienylcarbonyl, pyridylcarbonyl, pyrimidinylcarbonyl, pyridazinylcarbonyl, pyrazinylcarbonyl, triazinylcarbonyl, pyranylcarbonyl, dioxanylcarbonyl), sulfonyl (e.g., —S(═O)₂⁻), C3 to C6 cycloalkyl sulfonyl (e.g., cyclopropylsulfonyl, cyclobutylsulfonyl, cyclopentylsulfonyl, cyclohexylsulfonyl), C1 to C6 alkylsulfonyl (e.g., methylsulfonyl, ethylsulfonyl, n-propylsulfonyl, isopropylsulfonyl, n-butylsulfonyl, isobutylsulfonyl, tert-butylsulfonyl, n-pentylsulfonyl, isopentylsulfonyl, n-hexylsulfonyl, 2-methylpentylsulfonyl, 2,2-dimethylbutylsulfonyl, 2,3-dimethylbutylsulfonyl), C1 to C6 haloalkylsulfonyl (e.g., monofluoromethylsulfonyl, difluoromethylsulfonyl, trifluoromethylsulfonyl, dichloromethylsulfonyl, trichloromethylsulfonyl, monofluoroethylsulfonyl, difluoroethylsulfonyl, trifluoroethylsulfonyl, tetrafluoroethylsulfonyl, pentafluoroethylsulfonyl, dichloroethylsulfonyl, trichloroethylsulfonyl, tetrachloroethylsulfonyl, pentachloroethylsulfonyl, difluoropropylsulfonyl, trifluoropropylsulfonyl, tetrafluoropropylsulfonyl, pentafluoropropylsulfonyl, hexafluoropropylsulfonyl, perfluoropropylsulfonyl, monochloropropylsulfonyl, dichloropropylsulfonyl, trichloropropylsulfonyl, tetrachloropropylsulfonyl, pentachloropropylsulfonyl, hexachloropropylsulfonyl, perchloropropylsulfonyl), carbonyl, cyano, and halogen atoms (e.g., fluorine, chlorine, bromine, iodine);
R⁷ is selected from hydrogen, C1 to C6 alkyl (e.g. methyl, ethyl, n-propyl, isopropyl, n-butyl, isobutyl, tert-butyl, n-pentyl, isopentyl, n-hexyl, 2-methylpentyl, 2,2-dimethylbutyl, 2,3-dimethylbutyl), C1 to C6 haloalkyl group (e.g. monofluoromethyl, difluoromethyl, trifluoromethyl, dichloromethyl, trichloromethyl, monofluoroethyl, difluoroethyl, trifluoroethyl, tetrafluoroethyl, pentafluoroethyl, dichloroethyl, trichloroethyl, tetrachloroethyl, pentachloroethyl, difluoropropyl, trifluoropropyl, tetrafluoropropyl, pentafluoropropyl, hexafluoropropyl, perfluoropropyl, monochloropropyl, dichloropropyl, trichloropropyl, tetrafluoropropyl, pentachloropropyl, hexachloropropyl, perchloropropyl), C1 to C6 alkoxy (e.g., methoxy, ethoxy, n-propoxy, isopropoxy, n-butoxy, isobutoxy, tert-butoxy, n-pentoxy, isopentoxy, n-hexyloxy, 2-methylpentoxy, 2,2-dimethylbutoxy, 2,3-dimethylbutoxy), C2 to C8 alkoxyalkyl (e.g., methoxymethyl, ethoxymethyl, propoxymethyl, butoxymethyl, pentoxymethyl, hexoxymethyl, heptoxymethyl, methoxyethyl, ethoxyethyl, propoxyethyl, butoxyethyl, pentoxyethyl, hexoxyethyl, methoxypropyl, ethoxypropyl, propoxypropyl, butoxypropyl, pentoxypropyl, methoxybutyl, ethoxybutyl, propoxybutyl, butoxybutyl, methoxy pentyl, ethoxypentyl, propoxy pentyl, methoxyhexyl, ethoxyhexyl, methoxyheptyl), sulfonyl (e.g., —S(═O)₂⁻), C3 to C6 cycloalkylsulfonyl (e.g. cyclopropylsulfonyl, cyclobutylsulfonyl, cyclopentylsulfonyl, cyclohexylsulfonyl), C1 to C6 alkylsulfonyl (e.g. methylsulfonyl, ethylsulfonyl, n-propylsulfonyl, isopropylsulfonyl, n-butylsulfonyl, isobutylsulfonyl, tert-butylsulfonyl, n-pentylsulfonyl, isopentylsulfonyl, n-hexylsulfonyl, 2-methyl pentylsulfonyl, 2,2-dimethylbutylsulfonyl, 2,3-dimethylbutylsulfonyl), C1 to C6 haloalkylsulfonyl (e.g. monofluoromethylsulfonyl, difluoromethylsulfonyl, trifluoromethylsulfonyl, dichloromethylsulfonyl, trichloromethylsulfonyl, monofluoroethylsulfonyl, difluoroethylsulfonyl, trifluoroethylsulfonyl, tetrafluoroethylsulfonyl, pentafluoroethylsulfonyl, dichloroethylsulfonyl, trichloroethylsulfonyl, tetrachloroethylsulfonyl, pentachloroethylsulfonyl, difluoropropylsulfonyl, trifluoropropylsulfonyl, tetrafluoropropylsulfonyl, pentafluoropropylsulfonyl, hexafluoropropylsulfonyl, perfluoropropylsulfonyl, monochloropropylsulfonyl, dichloropropylsulfonyl, trichloropropylsulfonyl, tetrachloropropylsulfonyl, pentachloropropylsulfonyl, hexachloropropylsulfonyl, perchloropropylsulfonyl), C1 to C6 alkylacyl (e.g., methylacyl, ethylacyl, n-propylacyl, isopropylacyl, n-butylacyl, isobutylacyl, tert-butylacyl, n-pentylacyl, isopentylacyl, n-hexylacyl, 2-methylpentylacyl, 2,2-dimethylbutylacyl, 2,3-dimethylbutylacyl), C1 to C6 haloalkylacyl (e.g., monofluoromethylacyl, difluoromethylacyl, trifluoromethylacyl, dichloromethylacyl, trichloromethylacyl, monofluoroethylacyl, difluoroethylacyl, trifluoroethylacyl, tetrafluoroethylacyl, pentafluoroethylacyl, dichloroethylacyl, trichloroethylacyl, tetrachloroethylacyl, pentachloroethylacyl, difluoropropylacyl, trifluoropropylacyl, tetrafluoropropylacyl, pentafluoropropylacyl, hexafluoropropylacyl, perfluoropropylacyl, monochloropropylacyl, dichloropropylacyl, trichloropropylacyl, tetrachloropropylacyl, pentachloropropylacyl, hexachloropropylacyl, perchloropropylacyl), optionally substituted C6 to C15 arylacyl (e.g., phenylacyl, methylphenylacyl, ethylphenyl acyl, propylphenylacyl, butylphenylacyl, dimethylphenylacyl, methylethylphenylacyl, diethylphenylacyl, naphthylacyl, anthrylacyl), optionally substituted 5 to 15 membered heteroaryl acyl containing 1 to 3 heteroatoms selected from N, O, and S (e.g., furylacyl, thienylacyl, pyrrolylacyl, pyridylacyl, benzofurylacyl, benzothienylacyl, benzopyrrolylacyl, quinolinylacyl, isoquinolinylacyl), carbonyl, cyano, halogen atom (e.g., fluorine, chlorine, bromine, iodine), and C1 to C6 alkoxy substituted by 4 to 6 membered heterocycloalkyl containing 1 to 3 heteroatoms selected from N, O, and S (e.g., oxetanylmethoxy, azetidinylmethoxy, thietanylmethoxy, furylmethoxy, tetrahydrofurylmethoxy, pyrrolylmethyoxy, tetrahydropyrrolylmethoxy, oxazolylmethoxy, thiazolylmethoxy, imidazolylmethoxy, pyrazolylmethoxy, thienylmethoxy, pyridylmethoxy, pyrimidinylmethoxy, pyridazinylmethoxy, pyrazinylmethoxy, triazinylmethoxy, pyranylmethoxy, dioxanylmethoxy, oxetanylethoxy, azetidinylethoxy, thietanylethoxy, furylethoxy, tetrahydrofurylethoxy, pyrrolylethoxy, tetrahydropyrrolylethoxy, oxazolylethoxy, thiazolylethoxy, imidazolylethoxy, pyrazolylethoxy, thienylethoxy, pyridylethoxy, pyrimidinylethoxy, pyridazinylethoxy, pyrazinylethoxy, triazinylethoxy, pyranylethoxy, dioxanylethoxy, oxetanylpropoxy, azetidinylpropoxy, thietanylpropoxy, furylpropoxy, tetrahydrofurylpropoxy, pyrrolylpropoxy, tetrahydropyrrolylpropoxy, oxazolylpropoxy, thiazolylpropoxy, imidazolylpropoxy, pyrazolylpropoxy, thienylpropoxy, pyridylpropoxy, pyrimidinylpropoxy, pyridazinylpropoxy, pyrazinylpropoxy, triazinylpropoxy, pyranylpropoxy, dioxanylpropoxy, oxetanylbutoxy, azetidinylbutoxy, thietanylbutoxy, furylbutoxy, tetrahydrofurylbutoxy, pyrrolylbutoxy, tetrahydropyrrolylbutoxy, oxazolylbutoxy, thiazolylbutoxy, imidazolylbutoxy, pyrazolylbutoxy, thienylbutoxy, pyridylbutoxy, pyrimidinylbutoxy, pyridazinylbutoxy, pyrazinylbutoxy, triazinylbutoxy, pyranylbutoxy, dioxanylbutoxy).

The subscripts n3 and n4 are each independently an integer from 0 to 4.

In one embodiment, preferably, n3 and n4 are each independently 0, 1, 2, 3, or 4, respectively.

In one embodiment, preferably, n3 and n4 are each independently 0, 1, 2, or 3, respectively.

In one embodiment, preferably, n3 and n4 are each independently 0, 1, or 2, respectively.

In one embodiment, preferably, X² to X⁸ are each independently selected from C, O, and N, and X² to X⁸ each independently bond with 1 or 2 hydrogen atoms or without hydrogen atoms attached according to the bonding valence.

In one embodiment, preferably, R⁷ is selected from hydrogen, C1 to C4 alkyl, C1 to C4 haloalkyl, C1 to C4 alkoxy, C2 to C5 alkoxyalkyl, sulfonyl, C1 to C4 alkylsulfonyl, C1 to C4 haloalkylsulfonyl, carbonyl, cyano, halogen atom, and C1 to C4 alkoxy substituted by 4 to 6 membered heterocycloalkyl containing 1 to 3 heteroatoms selected from N, O, and S.

In one embodiment, the compound is selected from the following specific compounds:

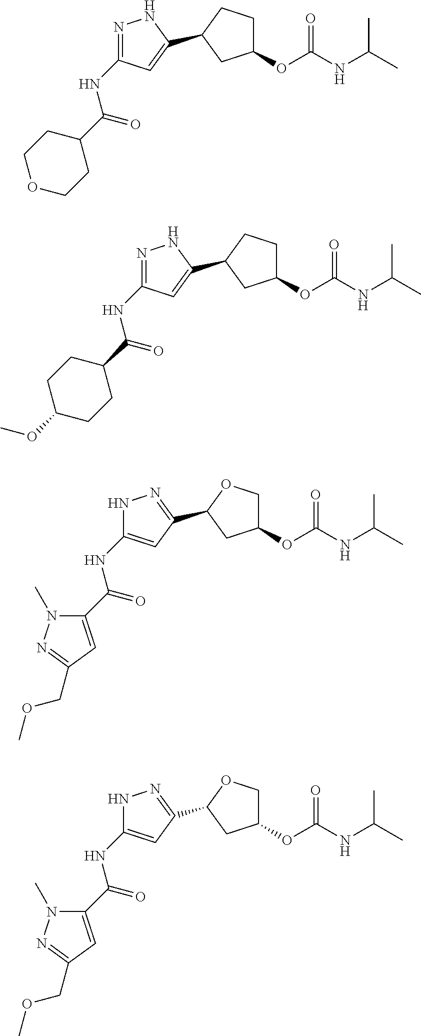

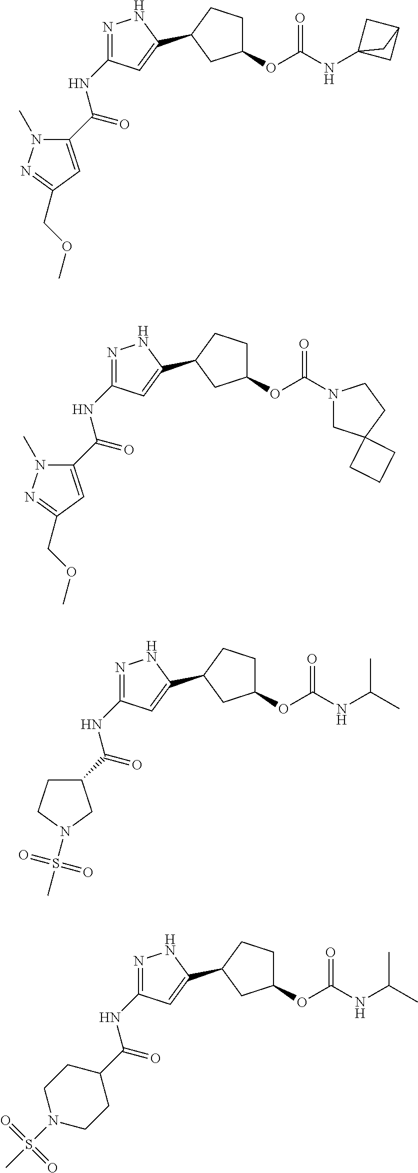

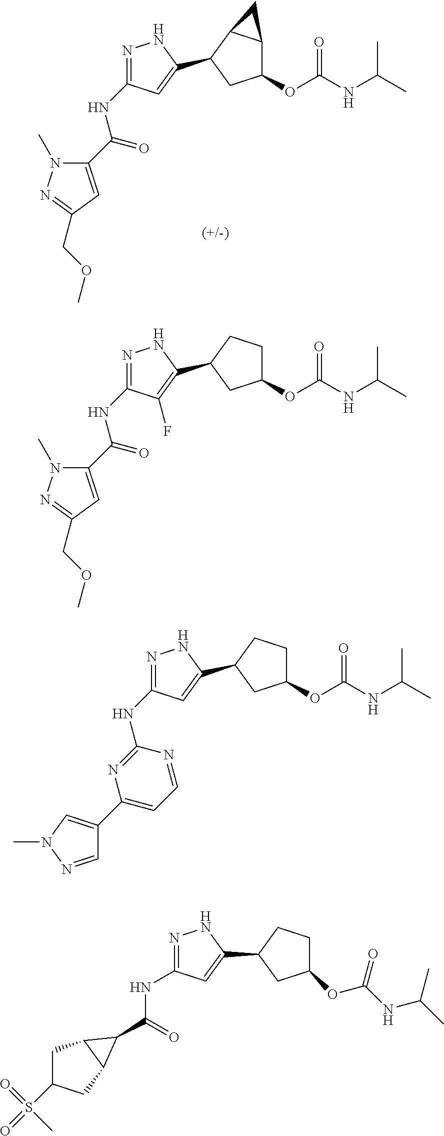

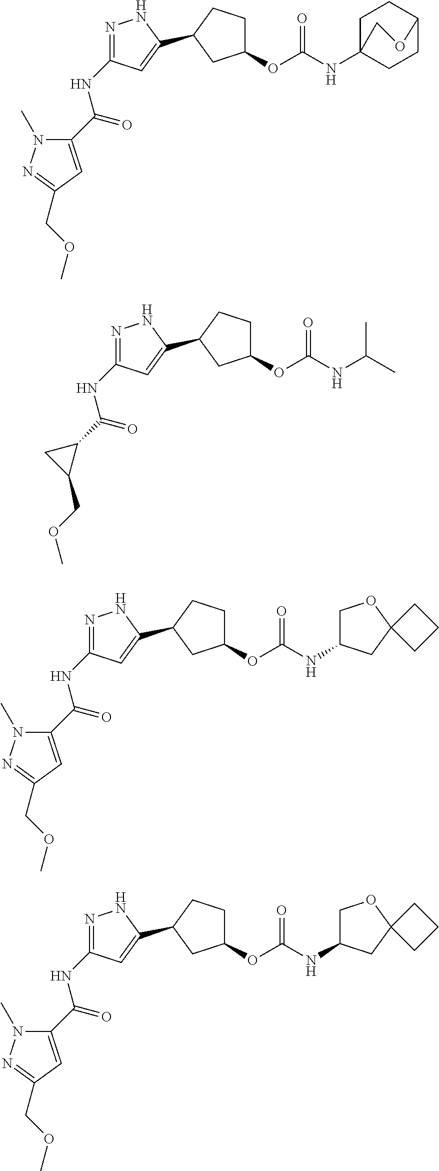

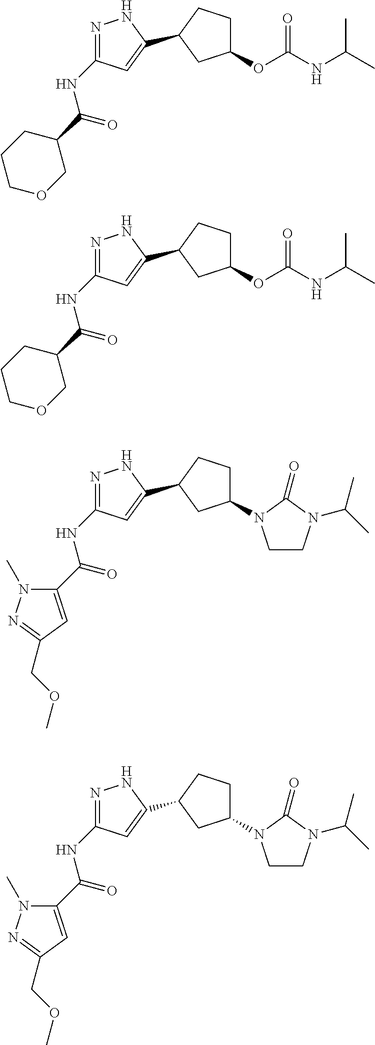

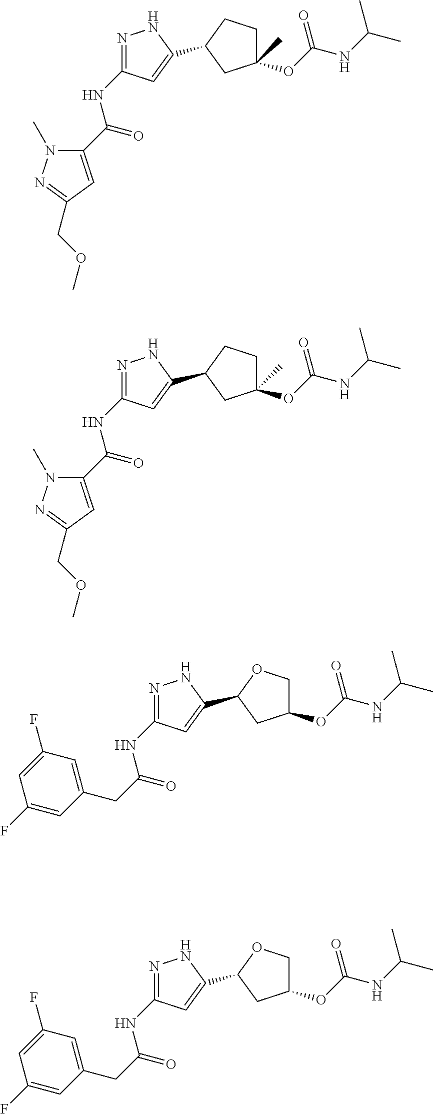

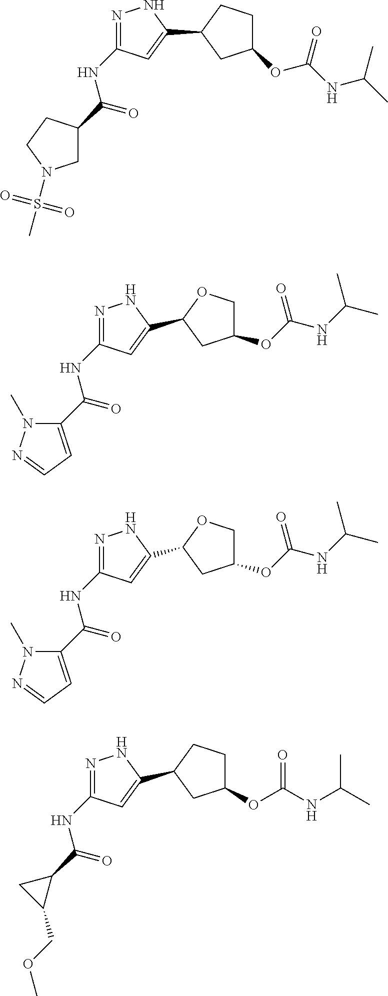

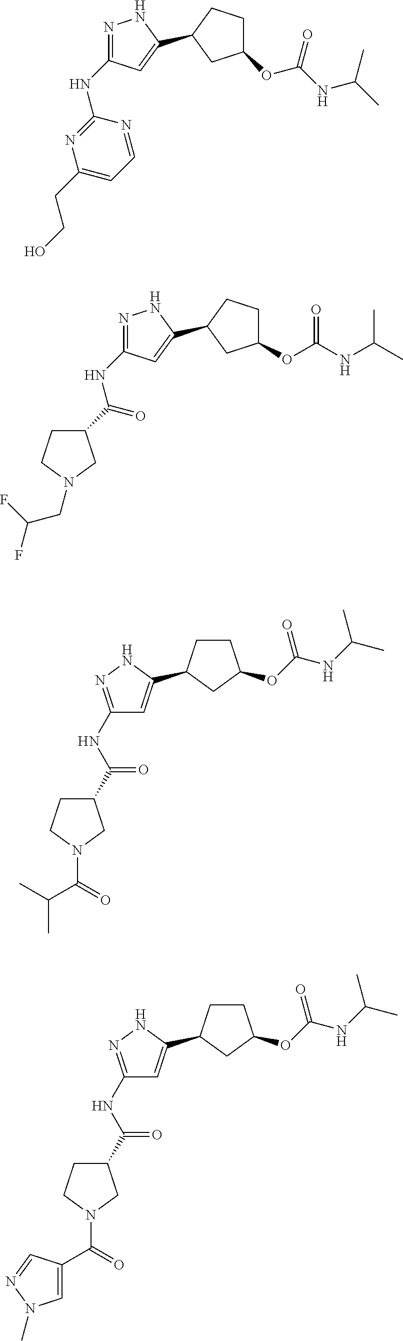

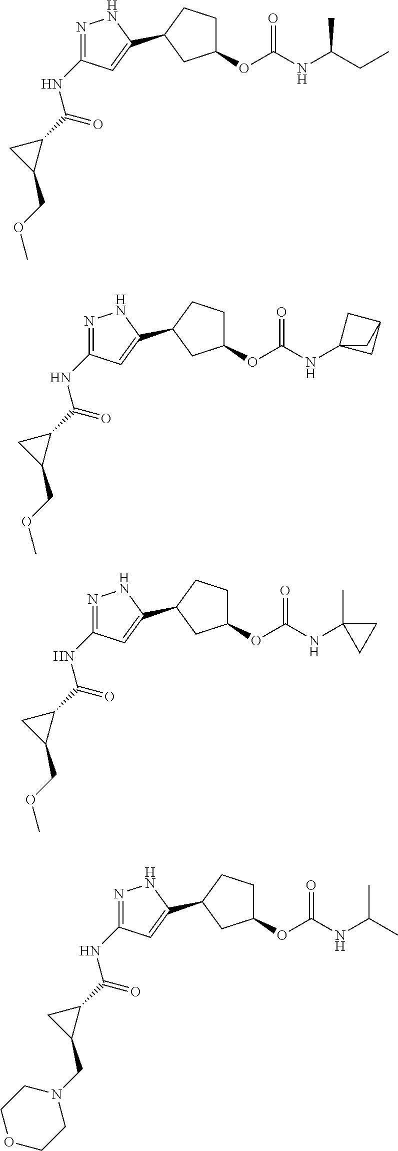 [SMILES: CC[C@H](C)NC(=O)O[C@@H]1CC[C@H](c2cc(NC(=O)[C@H]3C[C@@H]3COC)n[nH]2)C1 ; COC[C@H]1C[C@@H]1C(=O)Nc1cc([C@H]2CC[C@@H](OC(=O)NC34CC(C3)C4)C2)[nH]n1 ; COC[C@H]1C[C@@H]1C(=O)Nc1cc([C@H]2CC[C@@H](OC(=O)NC3(C)CC3)C2)[nH]n1 ; CC(C)NC(=O)O[C@@H]1CC[C@H](c2cc(NC(=O)[C@H]3C[C@@H]3CN3CCOCC3)n[nH]2)C1]

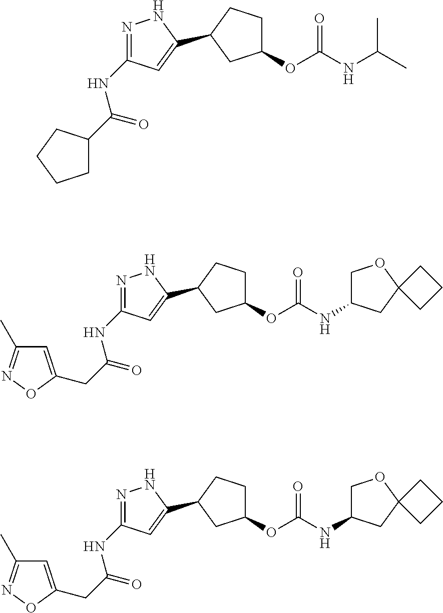

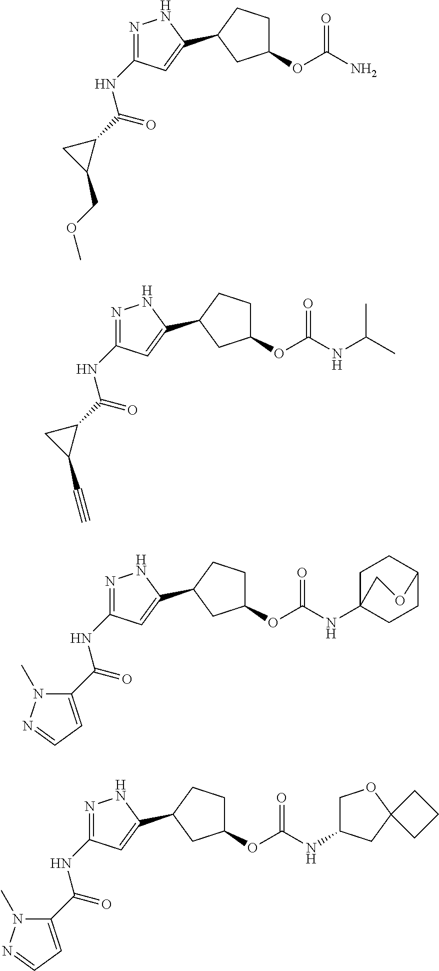

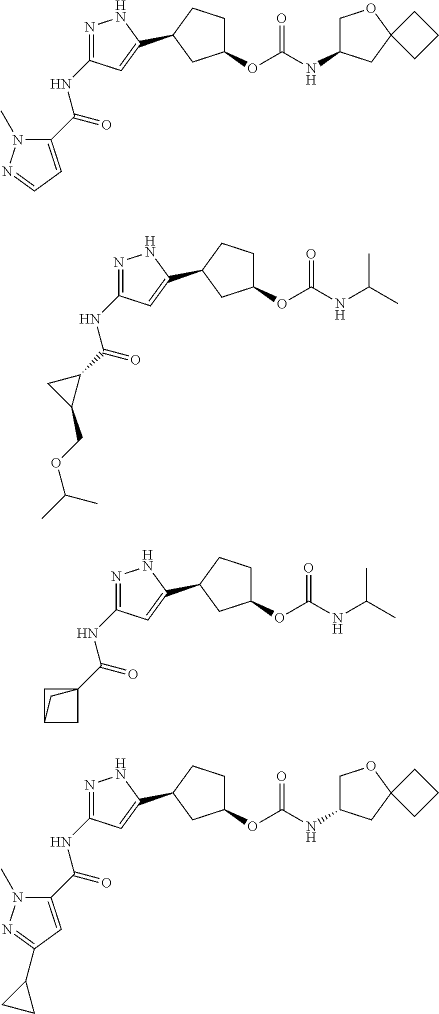

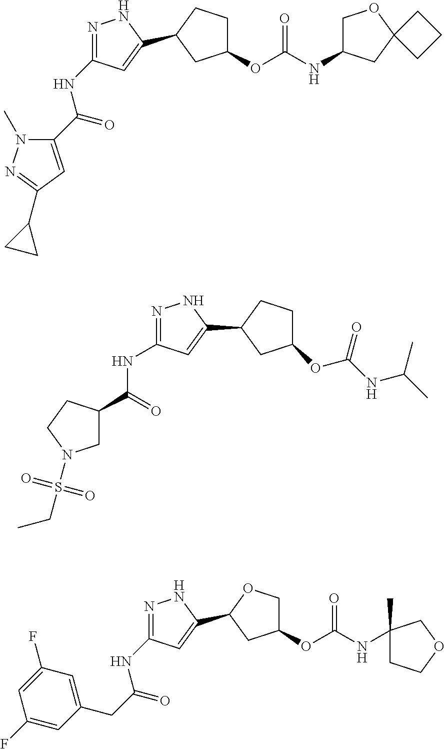 [SMILES: Cn1nc(C2CC2)cc1C(=O)Nc1cc([C@H]2CC[C@@H](OC(=O)N[C@H]3COC4(CCC4)C3)C2)[nH]n1 ; CCS(=O)(=O)N1CC[C@@H](C(=O)Nc2cc([C@H]3CC[C@@H](OC(=O)NC(C)C)C3)[nH]n2)C1 ; C[C@]1(NC(=O)O[C@@H]2CO[C@H](c3cc(NC(=O)Cc4cc(F)cc(F)c4)n[nH]3)C2)CCOC1]

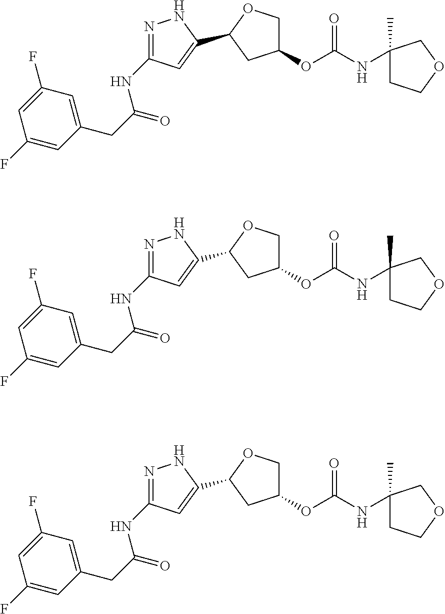 [SMILES: C[C@@]1(NC(=O)O[C@@H]2CO[C@H](c3cc(NC(=O)Cc4cc(F)cc(F)c4)n[nH]3)C2)CCOC1 ; C[C@]1(NC(=O)O[C@H]2CO[C@@H](c3cc(NC(=O)Cc4cc(F)cc(F)c4)n[nH]3)C2)CCOC1 ; C[C@@]1(NC(=O)O[C@H]2CO[C@@H](c3cc(NC(=O)Cc4cc(F)cc(F)c4)n[nH]3)C2)CCOC1]

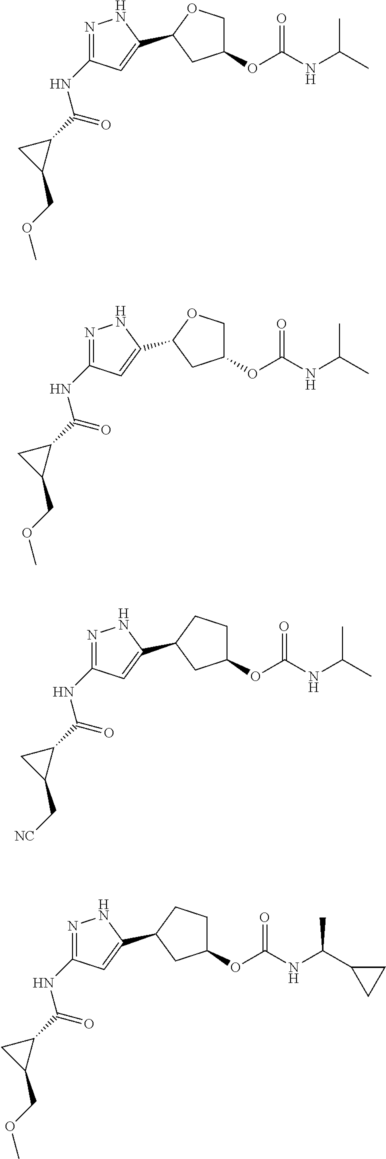 [SMILES: COC[C@H]1C[C@@H]1C(=O)Nc1cc([C@@H]2C[C@H](OC(=O)NC(C)C)CO2)[nH]n1 ; COC[C@H]1C[C@@H]1C(=O)Nc1cc([C@H]2C[C@@H](OC(=O)NC(C)C)CO2)[nH]n1 ; CC(C)NC(=O)O[C@@H]1CC[C@H](c2cc(NC(=O)[C@H]3C[C@@H]3CC#N)n[nH]2)C1 ; COC[C@H]1C[C@@H]1C(=O)Nc1cc([C@H]2CC[C@@H](OC(=O)N[C@@H](C)C3CC3)C2)[nH]n1]

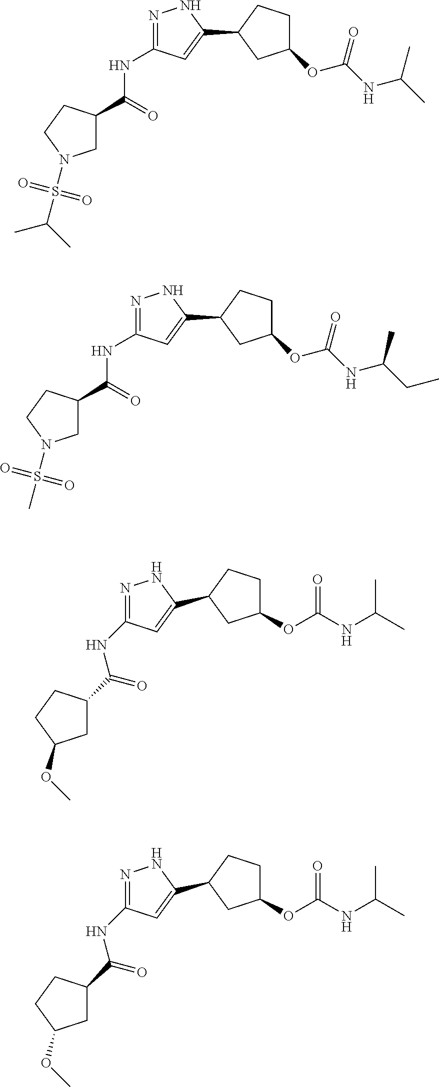 [SMILES: CC(C)NC(=O)O[C@@H]1CC[C@H](c2cc(NC(=O)[C@@H]3CCN(S(=O)(=O)C(C)C)C3)n[nH]2)C1 ; CC[C@H](C)NC(=O)O[C@@H]1CC[C@H](c2cc(NC(=O)[C@@H]3CCN(S(C)(=O)=O)C3)n[nH]2)C1 ; CO[C@H]1CC[C@H](C(=O)Nc2cc([C@H]3CC[C@@H](OC(=O)NC(C)C)C3)[nH]n2)C1 ; CO[C@@H]1CC[C@@H](C(=O)Nc2cc([C@H]3CC[C@@H](OC(=O)NC(C)C)C3)[nH]n2)C1]

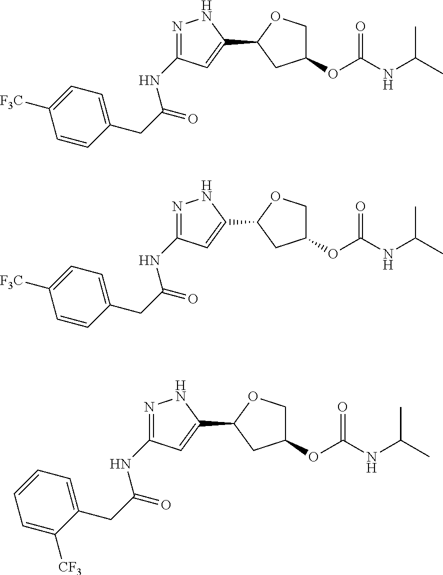 [SMILES: CC(C)NC(=O)O[C@@H]1CO[C@H](c2cc(NC(=O)Cc3ccc(C(F)(F)F)cc3)n[nH]2)C1 ; CC(C)NC(=O)O[C@H]1CO[C@@H](c2cc(NC(=O)Cc3ccc(C(F)(F)F)cc3)n[nH]2)C1 ; CC(C)NC(=O)O[C@@H]1CO[C@H](c2cc(NC(=O)Cc3ccccc3C(F)(F)F)n[nH]2)C1]

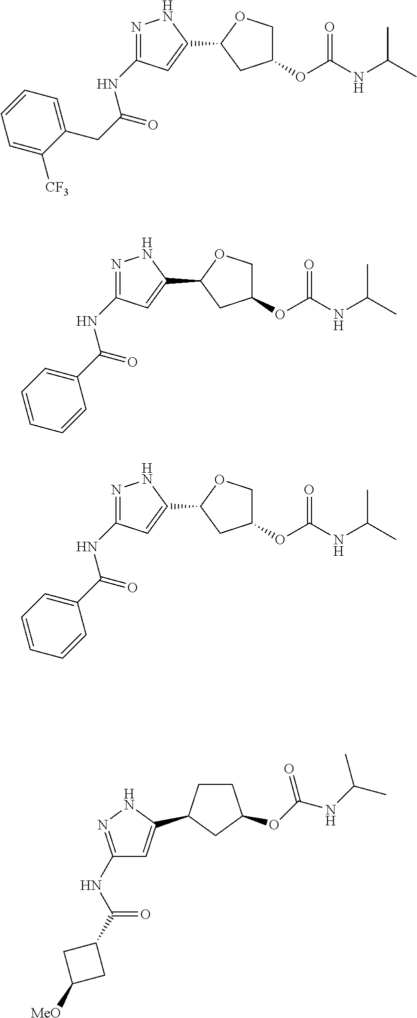 [SMILES: CC(C)NC(=O)O[C@H]1CO[C@@H](c2cc(NC(=O)Cc3ccccc3C(F)(F)F)n[nH]2)C1 ; CC(C)NC(=O)O[C@@H]1CO[C@H](c2cc(NC(=O)c3ccccc3)n[nH]2)C1 ; CC(C)NC(=O)O[C@H]1CO[C@@H](c2cc(NC(=O)c3ccccc3)n[nH]2)C1 ; CO[C@H]1C[C@H](C(=O)Nc2cc([C@H]3CC[C@@H](OC(=O)NC(C)C)C3)[nH]n2)C1]

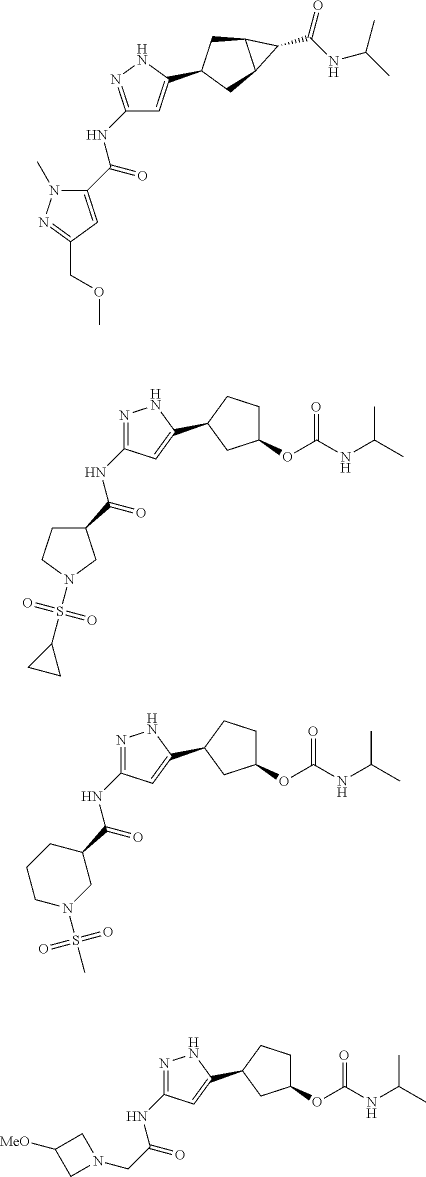 [SMILES: COCc1cc(C(=O)Nc2cc([C@H]3C[C@@H]4[C@H](C3)[C@H]4C(=O)NC(C)C)[nH]n2)n(C)n1 ; CC(C)NC(=O)O[C@@H]1CC[C@H](c2cc(NC(=O)[C@@H]3CCN(S(=O)(=O)C4CC4)C3)n[nH]2)C1 ; CC(C)NC(=O)O[C@@H]1CC[C@H](c2cc(NC(=O)[C@@H]3CCCN(S(C)(=O)=O)C3)n[nH]2)C1 ; COC1CN(CC(=O)Nc2cc([C@H]3CC[C@@H](OC(=O)NC(C)C)C3)[nH]n2)C1]

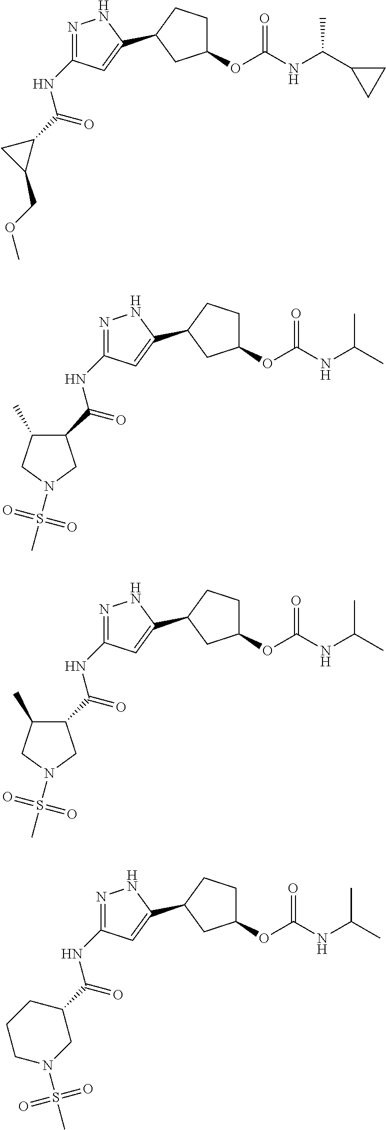 [SMILES: COC[C@H]1C[C@@H]1C(=O)Nc1cc([C@H]2CC[C@@H](OC(=O)N[C@H](C)C3CC3)C2)[nH]n1 ; CC(C)NC(=O)O[C@@H]1CC[C@H](c2cc(NC(=O)[C@H]3CN(S(C)(=O)=O)C[C@@H]3C)n[nH]2)C1 ; CC(C)NC(=O)O[C@@H]1CC[C@H](c2cc(NC(=O)[C@@H]3CN(S(C)(=O)=O)C[C@H]3C)n[nH]2)C1 ; CC(C)NC(=O)O[C@@H]1CC[C@H](c2cc(NC(=O)[C@H]3CCCN(S(C)(=O)=O)C3)n[nH]2)C1]

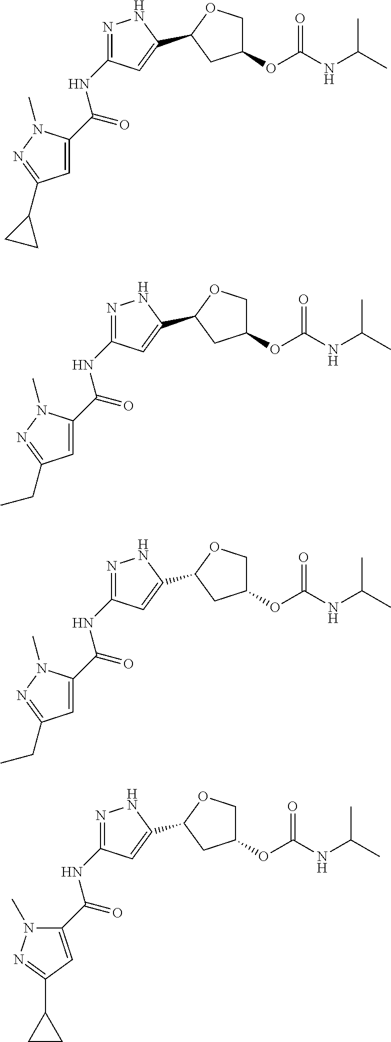

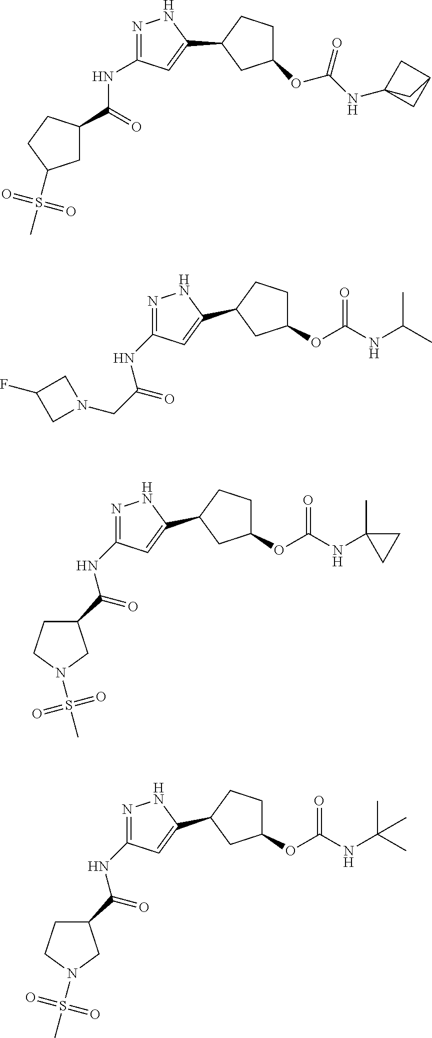 [SMILES: CS(=O)(=O)C1CC[C@@H](C(=O)Nc2cc([C@H]3CC[C@@H](OC(=O)NC45CC(C4)C5)C3)[nH]n2)C1 ; CC(C)NC(=O)O[C@@H]1CC[C@H](c2cc(NC(=O)CN3CC(F)C3)n[nH]2)C1 ; CC1(NC(=O)O[C@@H]2CC[C@H](c3cc(NC(=O)[C@@H]4CCN(S(C)(=O)=O)C4)n[nH]3)C2)CC1 ; CC(C)(C)NC(=O)O[C@@H]1CC[C@H](c2cc(NC(=O)[C@@H]3CCN(S(C)(=O)=O)C3)n[nH]2)C1]

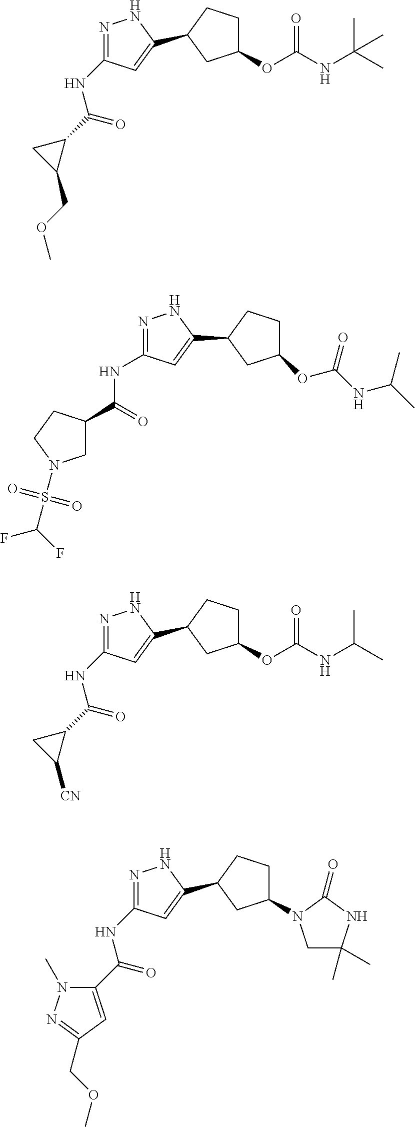

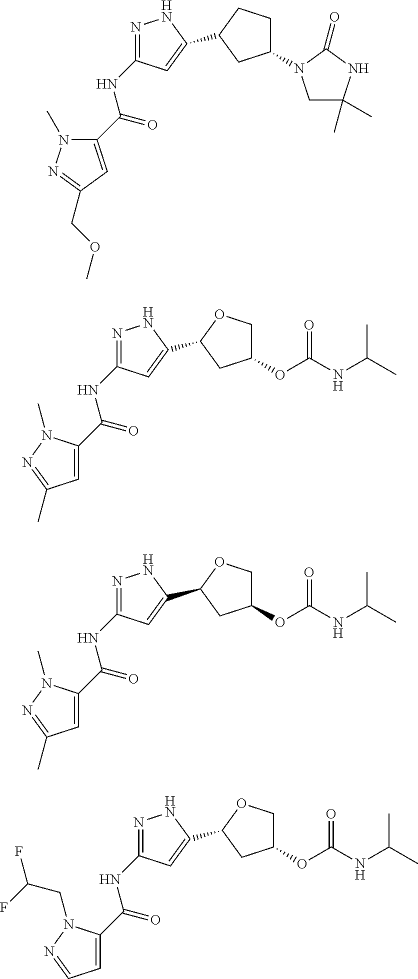

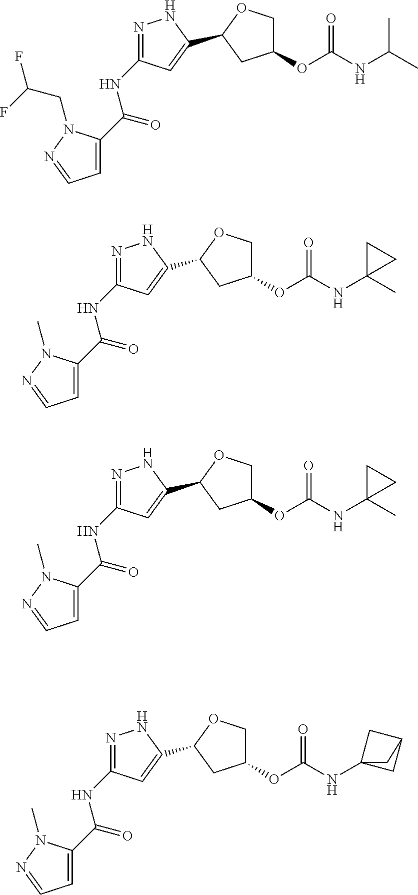 [SMILES: CC(C)NC(=O)O[C@@H]1CO[C@H](c2cc(NC(=O)c3ccnn3CC(F)F)n[nH]2)C1 ; Cn1nccc1C(=O)Nc1cc([C@H]2C[C@@H](OC(=O)NC3(C)CC3)CO2)[nH]n1 ; Cn1nccc1C(=O)Nc1cc([C@@H]2C[C@H](OC(=O)NC3(C)CC3)CO2)[nH]n1 ; Cn1nccc1C(=O)Nc1cc([C@H]2C[C@@H](OC(=O)NC34CC(C3)C4)CO2)[nH]n1]

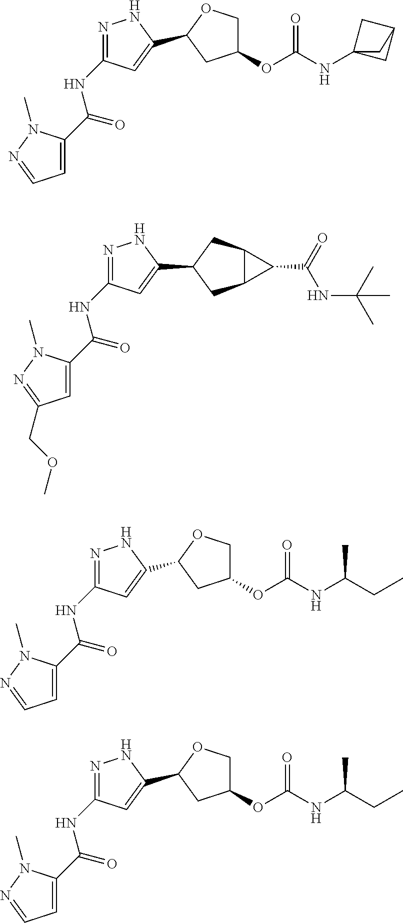 [SMILES: Cn1nccc1C(=O)Nc1cc([C@@H]2C[C@H](OC(=O)NC34CC(C3)C4)CO2)[nH]n1 ; COCc1cc(C(=O)Nc2cc([C@H]3C[C@@H]4[C@H](C3)[C@H]4C(=O)NC(C)(C)C)[nH]n2)n(C)n1 ; CC[C@H](C)NC(=O)O[C@H]1CO[C@@H](c2cc(NC(=O)c3ccnn3C)n[nH]2)C1 ; CC[C@H](C)NC(=O)O[C@@H]1CO[C@H](c2cc(NC(=O)c3ccnn3C)n[nH]2)C1]

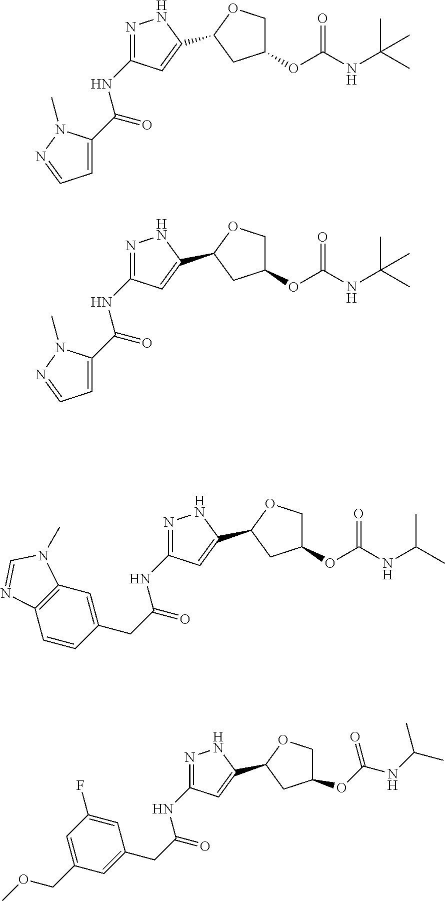

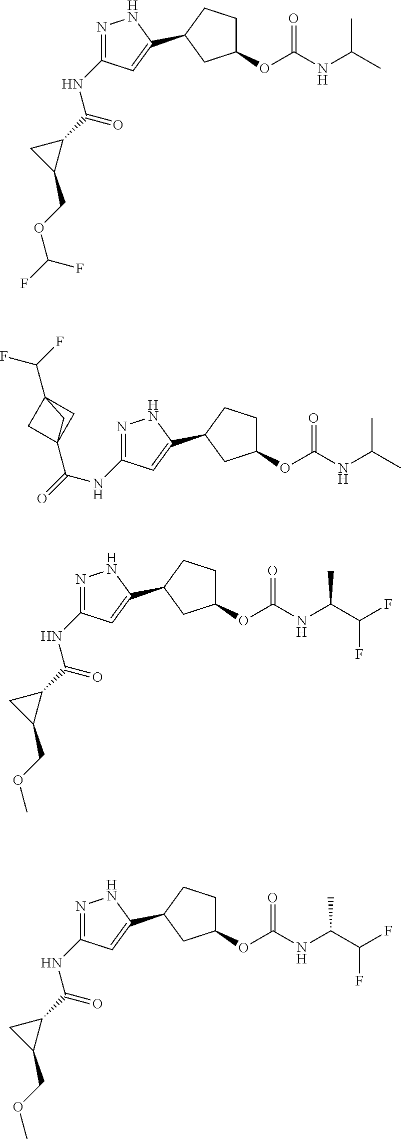 [SMILES: CC(C)NC(=O)O[C@@H]1CC[C@H](c2cc(NC(=O)[C@H]3C[C@@H]3COC(F)F)n[nH]2)C1 ; CC(C)NC(=O)O[C@@H]1CC[C@H](c2cc(NC(=O)C34CC(C(F)F)(C3)C4)n[nH]2)C1 ; COC[C@H]1C[C@@H]1C(=O)Nc1cc([C@H]2CC[C@@H](OC(=O)N[C@@H](C)C(F)F)C2)[nH]n1 ; COC[C@H]1C[C@@H]1C(=O)Nc1cc([C@H]2CC[C@@H](OC(=O)N[C@H](C)C(F)F)C2)[nH]n1]

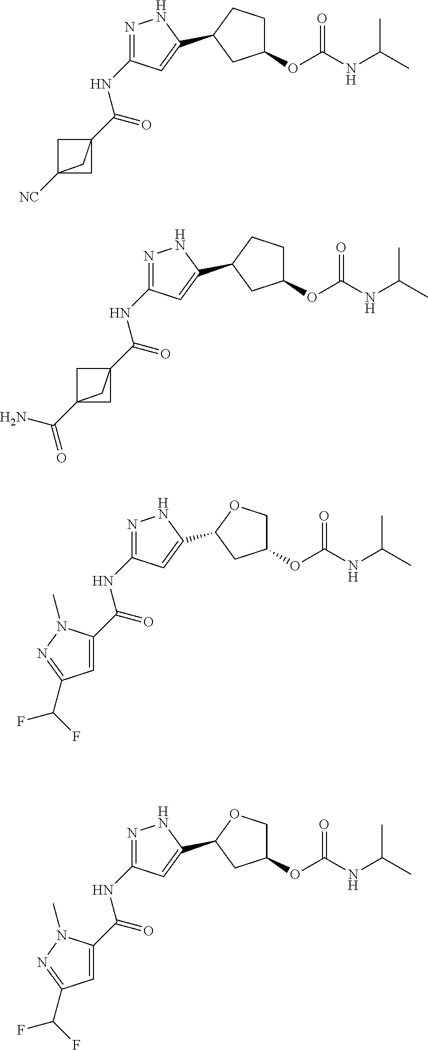 [SMILES: CC(C)NC(=O)O[C@@H]1CC[C@H](c2cc(NC(=O)C34CC(C#N)(C3)C4)n[nH]2)C1 ; CC(C)NC(=O)O[C@@H]1CC[C@H](c2cc(NC(=O)C34CC(C(N)=O)(C3)C4)n[nH]2)C1 ; CC(C)NC(=O)O[C@H]1CO[C@@H](c2cc(NC(=O)c3cc(C(F)F)nn3C)n[nH]2)C1 ; CC(C)NC(=O)O[C@@H]1CO[C@H](c2cc(NC(=O)c3cc(C(F)F)nn3C)n[nH]2)C1]

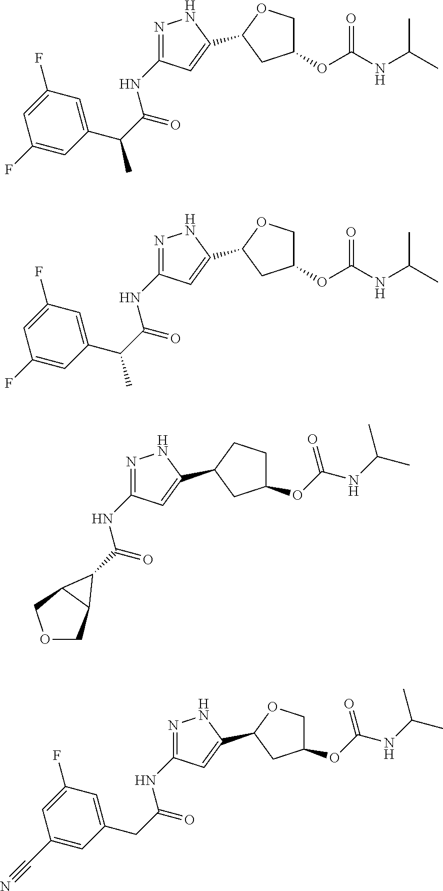 [SMILES: CC(C)NC(=O)O[C@H]1CO[C@@H](c2cc(NC(=O)[C@@H](C)c3cc(F)cc(F)c3)n[nH]2)C1 ; CC(C)NC(=O)O[C@H]1CO[C@@H](c2cc(NC(=O)[C@H](C)c3cc(F)cc(F)c3)n[nH]2)C1 ; CC(C)NC(=O)O[C@@H]1CC[C@H](c2cc(NC(=O)[C@H]3[C@@H]4COC[C@@H]43)n[nH]2)C1 ; CC(C)NC(=O)O[C@@H]1CO[C@H](c2cc(NC(=O)Cc3cc(F)cc(C#N)c3)n[nH]2)C1]

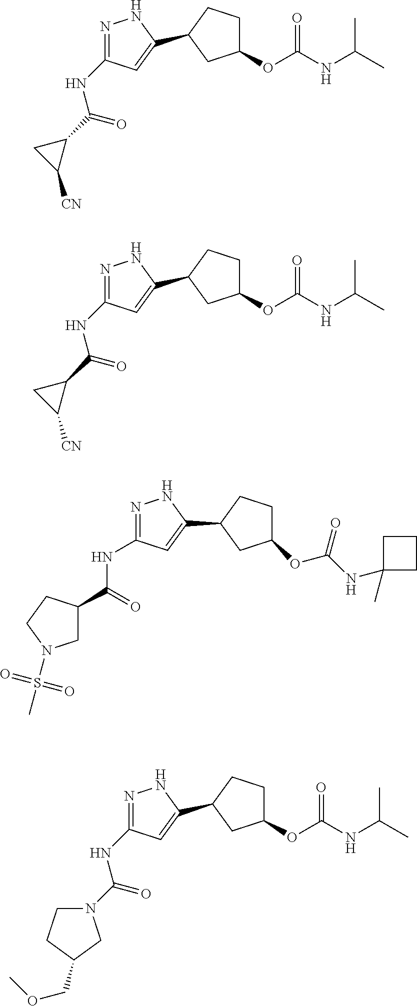

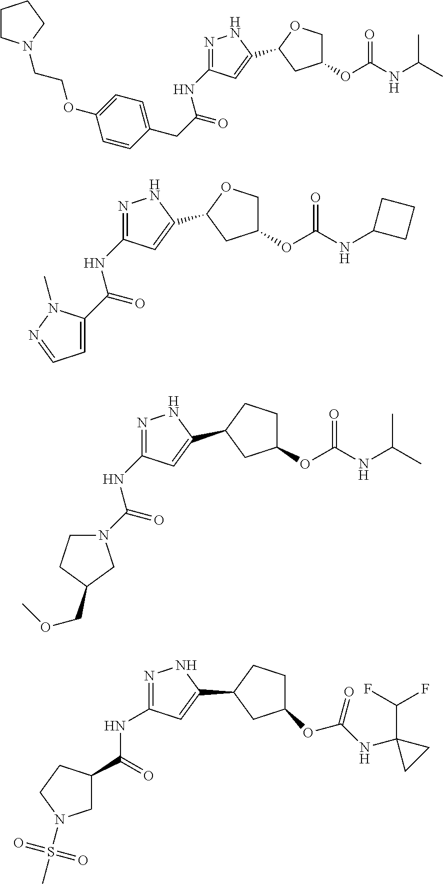

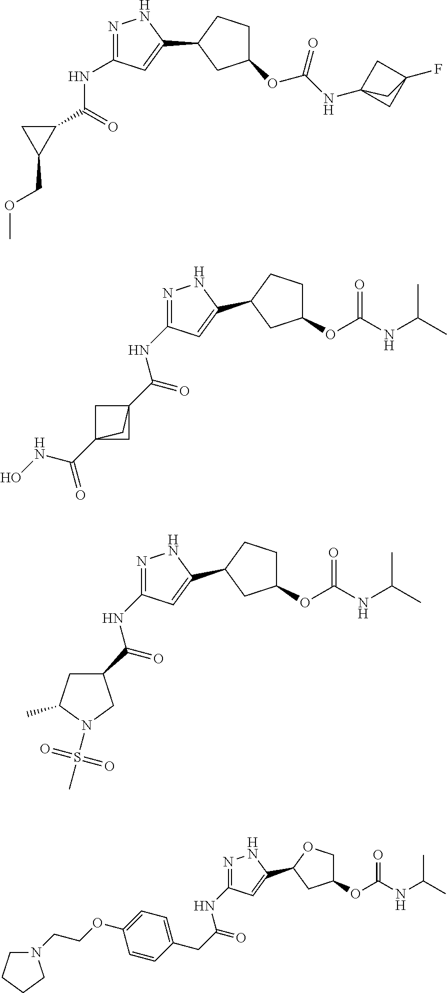

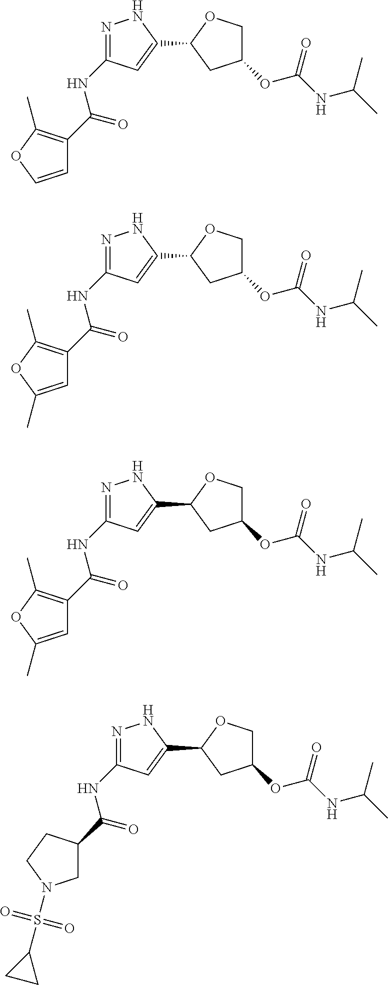 [SMILES: Cc1occc1C(=O)Nc1cc([C@H]2C[C@@H](OC(=O)NC(C)C)CO2)[nH]n1 ; Cc1cc(C(=O)Nc2cc([C@H]3C[C@@H](OC(=O)NC(C)C)CO3)[nH]n2)c(C)o1 ; Cc1cc(C(=O)Nc2cc([C@@H]3C[C@H](OC(=O)NC(C)C)CO3)[nH]n2)c(C)o1 ; CC(C)NC(=O)O[C@@H]1CO[C@H](c2cc(NC(=O)[C@@H]3CCN(S(=O)(=O)C4CC4)C3)n[nH]2)C1]

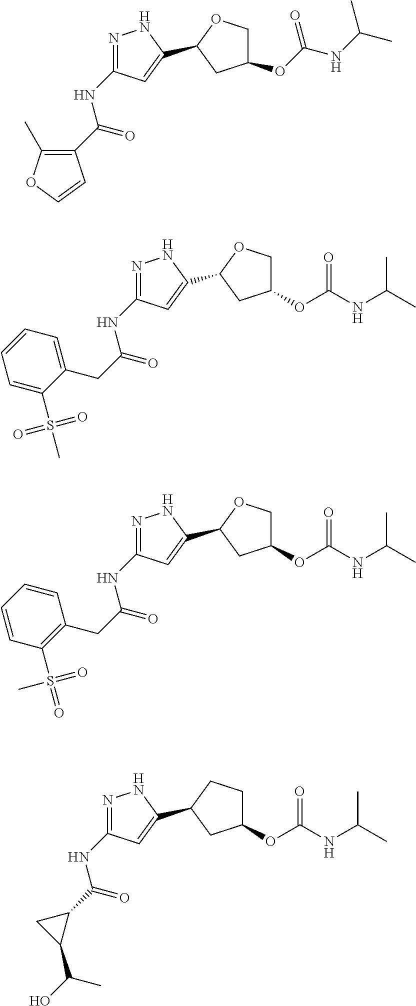 [SMILES: Cc1occc1C(=O)Nc1cc([C@@H]2C[C@H](OC(=O)NC(C)C)CO2)[nH]n1 ; CC(C)NC(=O)O[C@H]1CO[C@@H](c2cc(NC(=O)Cc3ccccc3S(C)(=O)=O)n[nH]2)C1 ; CC(C)NC(=O)O[C@@H]1CO[C@H](c2cc(NC(=O)Cc3ccccc3S(C)(=O)=O)n[nH]2)C1 ; CC(C)NC(=O)O[C@@H]1CC[C@H](c2cc(NC(=O)[C@H]3C[C@@H]3C(C)O)n[nH]2)C1]

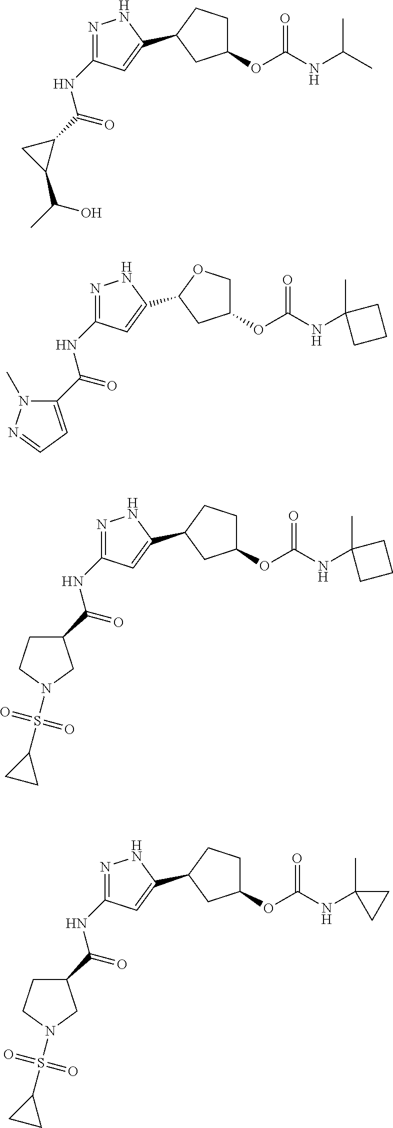

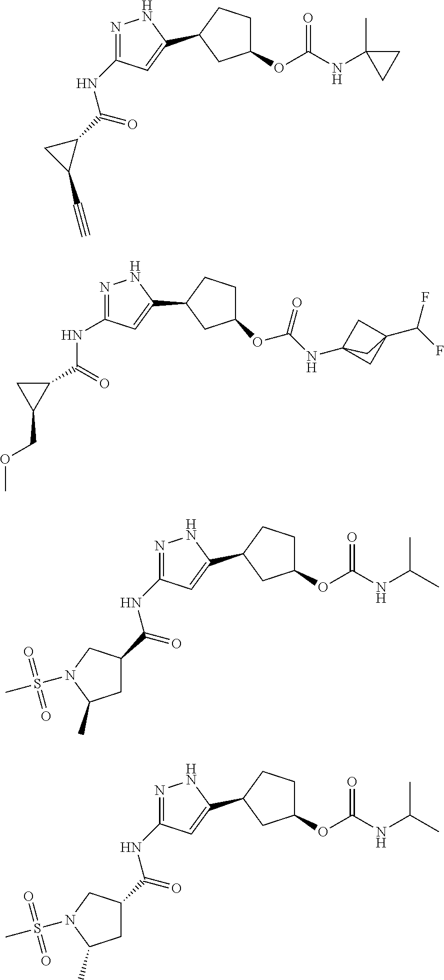

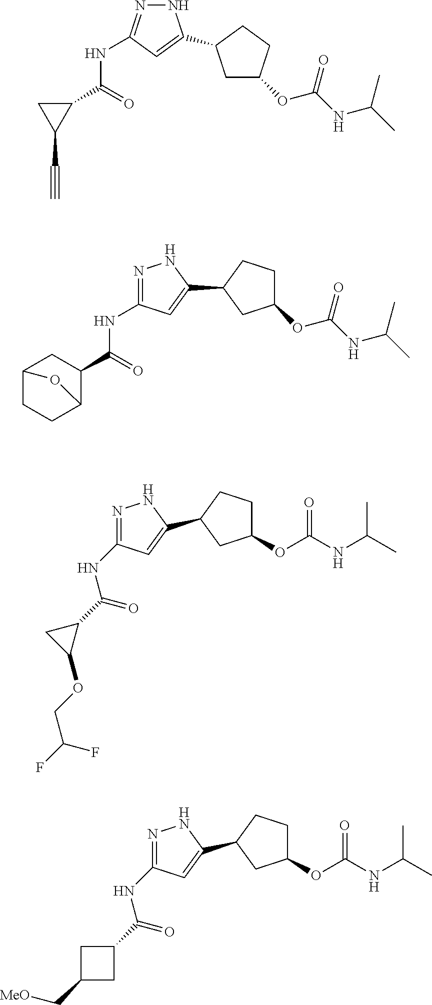

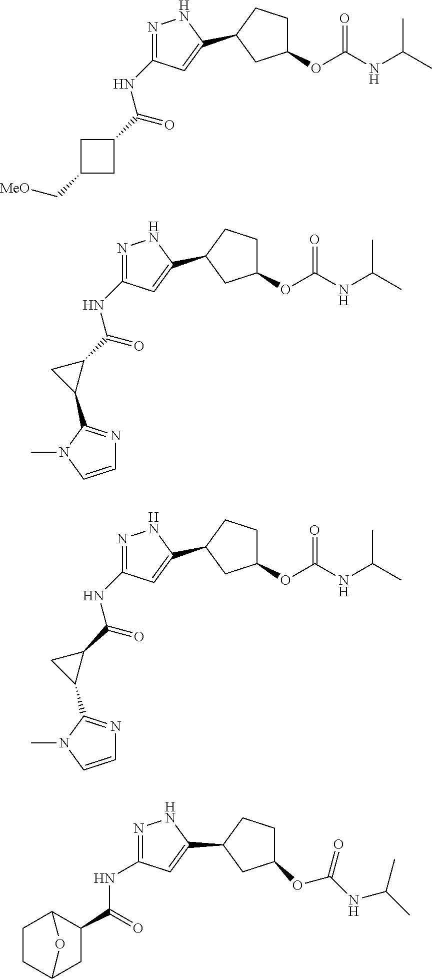

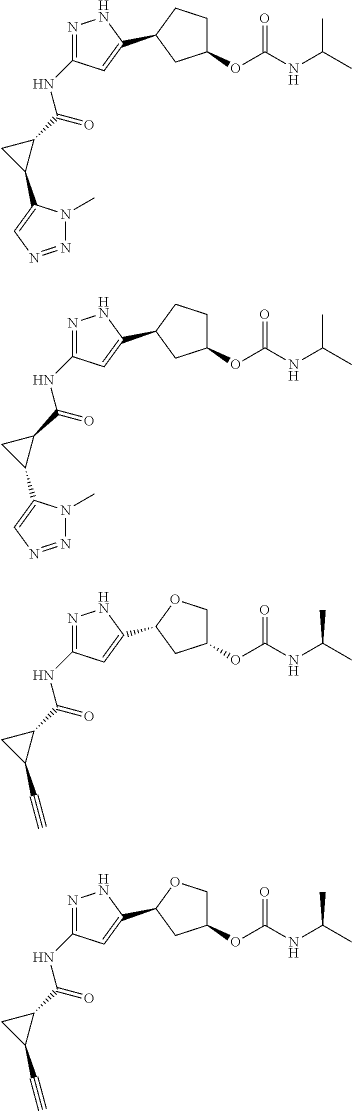

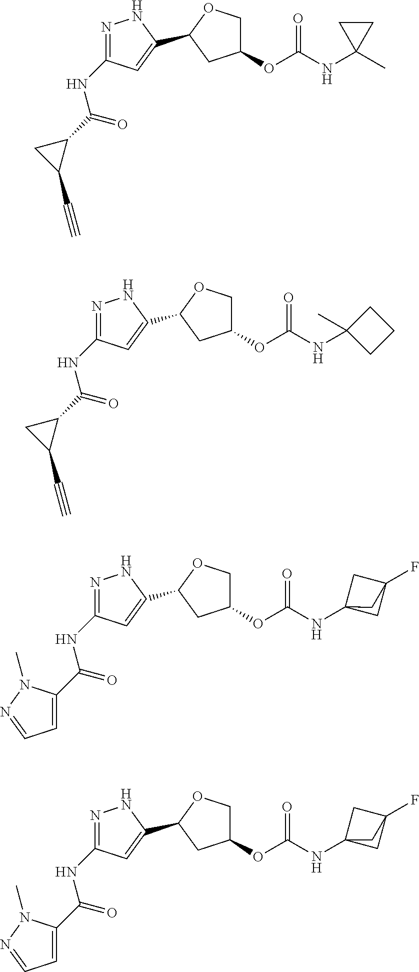 [SMILES: C#C[C@H]1C[C@@H]1C(=O)Nc1cc([C@@H]2C[C@H](OC(=O)NC3(C)CC3)CO2)[nH]n1 ; C#C[C@H]1C[C@@H]1C(=O)Nc1cc([C@H]2C[C@@H](OC(=O)NC3(C)CCC3)CO2)[nH]n1 ; Cn1nccc1C(=O)Nc1cc([C@H]2C[C@@H](OC(=O)NC34CC(F)(C3)C4)CO2)[nH]n1 ; Cn1nccc1C(=O)Nc1cc([C@@H]2C[C@H](OC(=O)NC34CC(F)(C3)C4)CO2)[nH]n1]

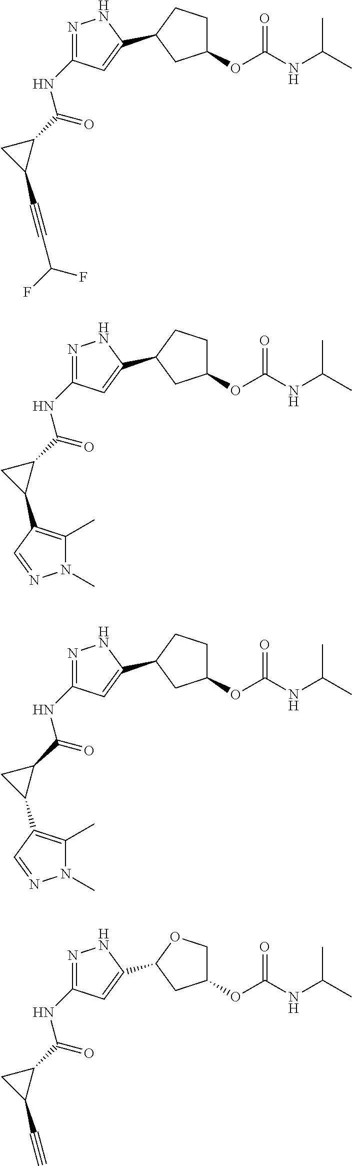 [SMILES: CC(C)NC(=O)O[C@@H]1CC[C@H](c2cc(NC(=O)[C@H]3C[C@@H]3C#CC(F)F)n[nH]2)C1 ; Cc1c([C@H]2C[C@@H]2C(=O)Nc2cc([C@H]3CC[C@@H](OC(=O)NC(C)C)C3)[nH]n2)cnn1C ; Cc1c([C@@H]2C[C@H]2C(=O)Nc2cc([C@H]3CC[C@@H](OC(=O)NC(C)C)C3)[nH]n2)cnn1C ; C#C[C@H]1C[C@@H]1C(=O)Nc1cc([C@H]2C[C@@H](OC(=O)NC(C)C)CO2)[nH]n1]

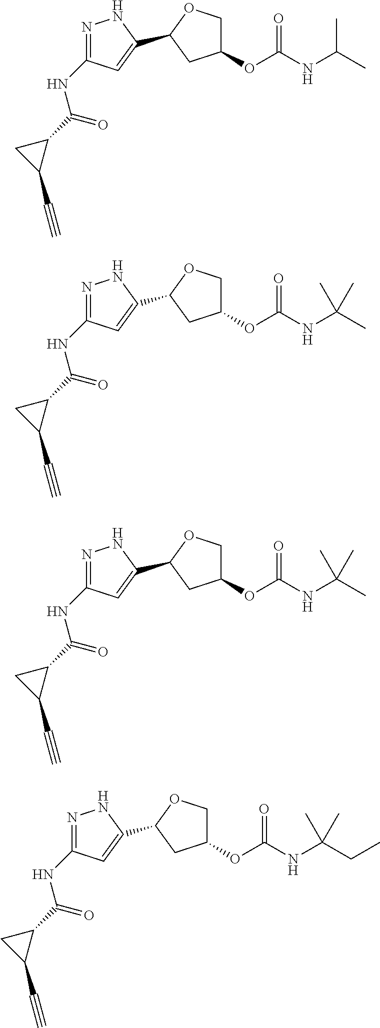 [SMILES: C#C[C@H]1C[C@@H]1C(=O)Nc1cc([C@@H]2C[C@H](OC(=O)NC(C)C)CO2)[nH]n1 ; C#C[C@H]1C[C@@H]1C(=O)Nc1cc([C@H]2C[C@@H](OC(=O)NC(C)(C)C)CO2)[nH]n1 ; C#C[C@H]1C[C@@H]1C(=O)Nc1cc([C@@H]2C[C@H](OC(=O)NC(C)(C)C)CO2)[nH]n1 ; C#C[C@H]1C[C@@H]1C(=O)Nc1cc([C@H]2C[C@@H](OC(=O)NC(C)(C)CC)CO2)[nH]n1]

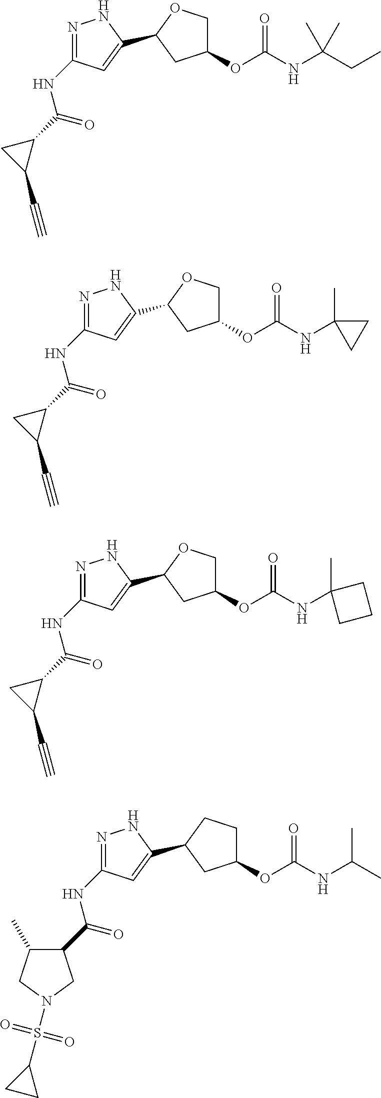

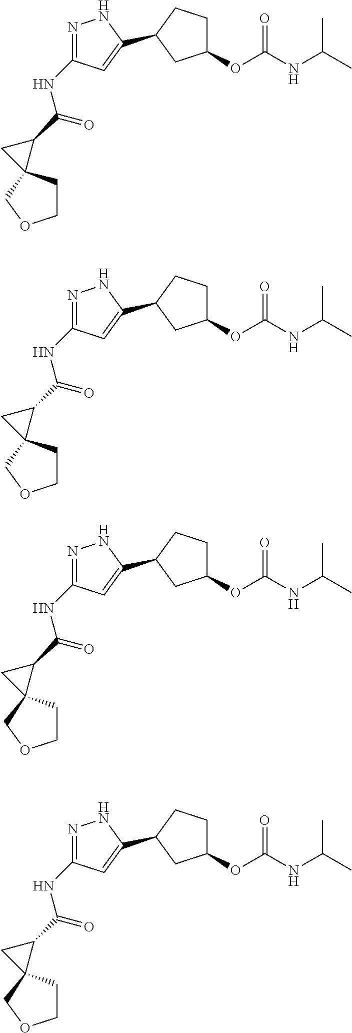 [SMILES: CC(C)NC(=O)O[C@@H]1CC[C@H](c2cc(NC(=O)[C@@H]3C[C@]34CCOC4)n[nH]2)C1 ; CC(C)NC(=O)O[C@@H]1CC[C@H](c2cc(NC(=O)[C@H]3C[C@]34CCOC4)n[nH]2)C1 ; CC(C)NC(=O)O[C@@H]1CC[C@H](c2cc(NC(=O)[C@@H]3C[C@@]34CCOC4)n[nH]2)C1 ; CC(C)NC(=O)O[C@@H]1CC[C@H](c2cc(NC(=O)[C@H]3C[C@@]34CCOC4)n[nH]2)C1]

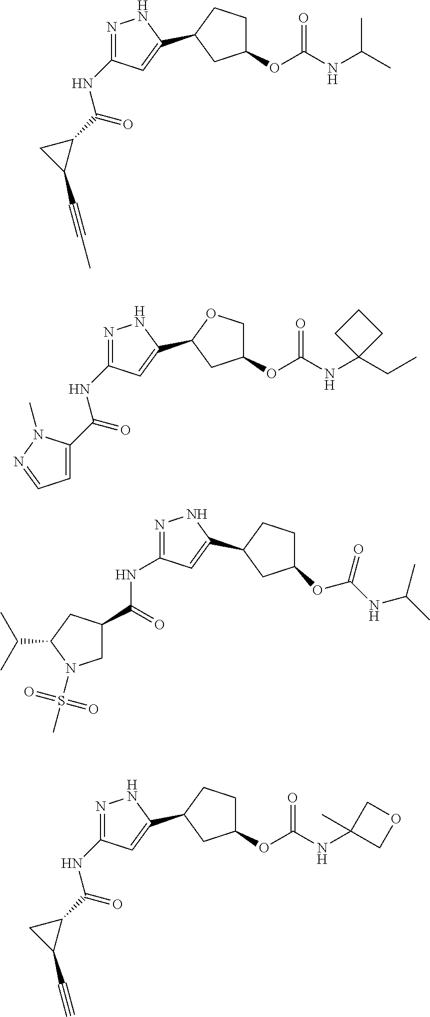

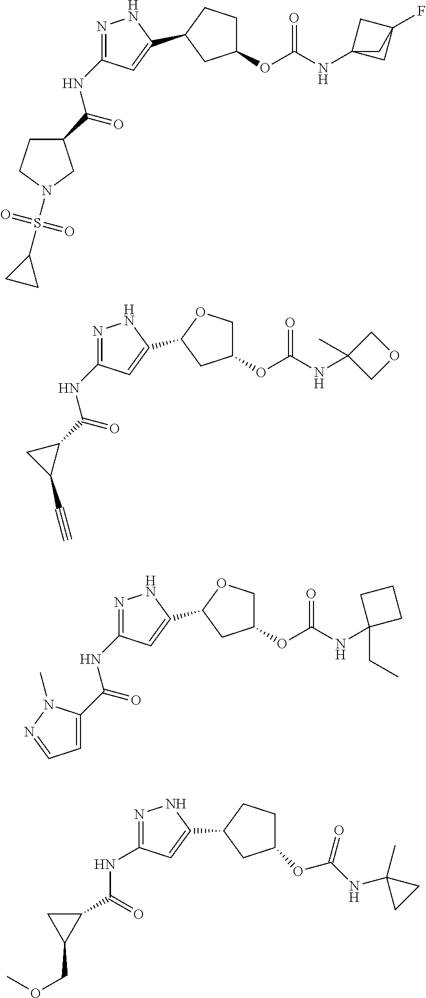 [SMILES: O=C(NC12CC(F)(C1)C2)O[C@@H]1CC[C@H](c2cc(NC(=O)[C@@H]3CCN(S(=O)(=O)C4CC4)C3)n[nH]2)C1 ; C#C[C@H]1C[C@@H]1C(=O)Nc1cc([C@H]2C[C@@H](OC(=O)NC3(C)COC3)CO2)[nH]n1 ; CCC1(NC(=O)O[C@H]2CO[C@@H](c3cc(NC(=O)c4ccnn4C)n[nH]3)C2)CCC1 ; COC[C@H]1C[C@@H]1C(=O)Nc1cc([C@@H]2CC[C@H](OC(=O)NC3(C)CC3)C2)[nH]n1]

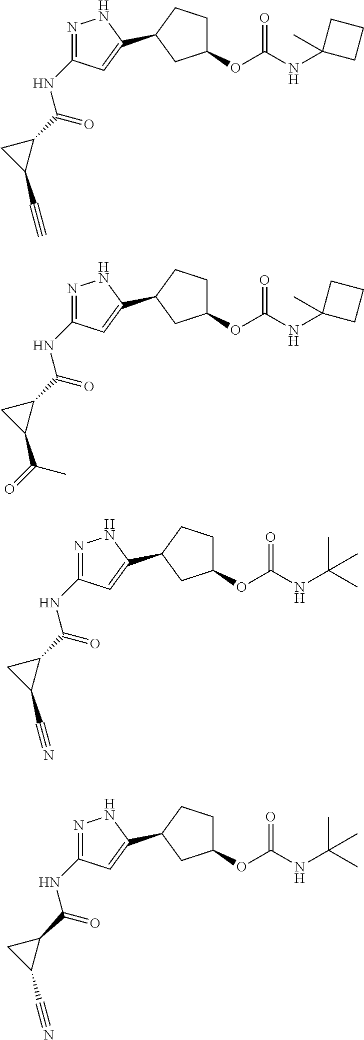 [SMILES: C#C[C@H]1C[C@@H]1C(=O)Nc1cc([C@H]2CC[C@@H](OC(=O)NC3(C)CCC3)C2)[nH]n1 ; CC(=O)[C@H]1C[C@@H]1C(=O)Nc1cc([C@H]2CC[C@@H](OC(=O)NC3(C)CCC3)C2)[nH]n1 ; CC(C)(C)NC(=O)O[C@@H]1CC[C@H](c2cc(NC(=O)[C@H]3C[C@@H]3C#N)n[nH]2)C1 ; CC(C)(C)NC(=O)O[C@@H]1CC[C@H](c2cc(NC(=O)[C@@H]3C[C@H]3C#N)n[nH]2)C1]

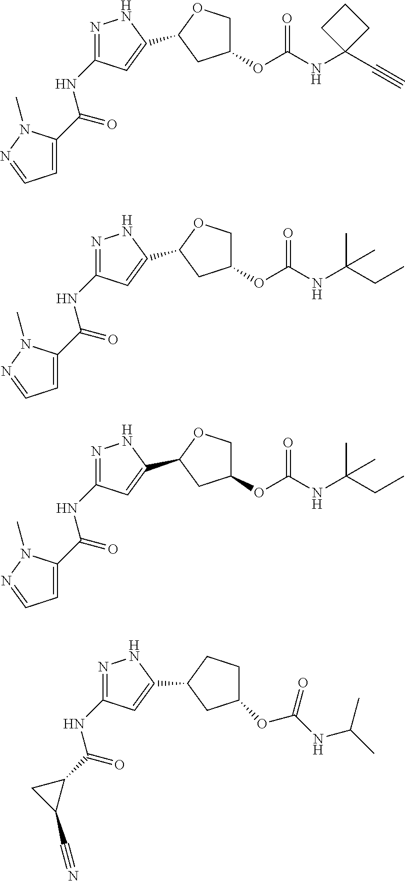 [SMILES: C#CC1(NC(=O)O[C@H]2CO[C@@H](c3cc(NC(=O)c4ccnn4C)n[nH]3)C2)CCC1 ; CCC(C)(C)NC(=O)O[C@H]1CO[C@@H](c2cc(NC(=O)c3ccnn3C)n[nH]2)C1 ; CCC(C)(C)NC(=O)O[C@@H]1CO[C@H](c2cc(NC(=O)c3ccnn3C)n[nH]2)C1 ; CC(C)NC(=O)O[C@H]1CC[C@@H](c2cc(NC(=O)[C@H]3C[C@@H]3C#N)n[nH]2)C1]

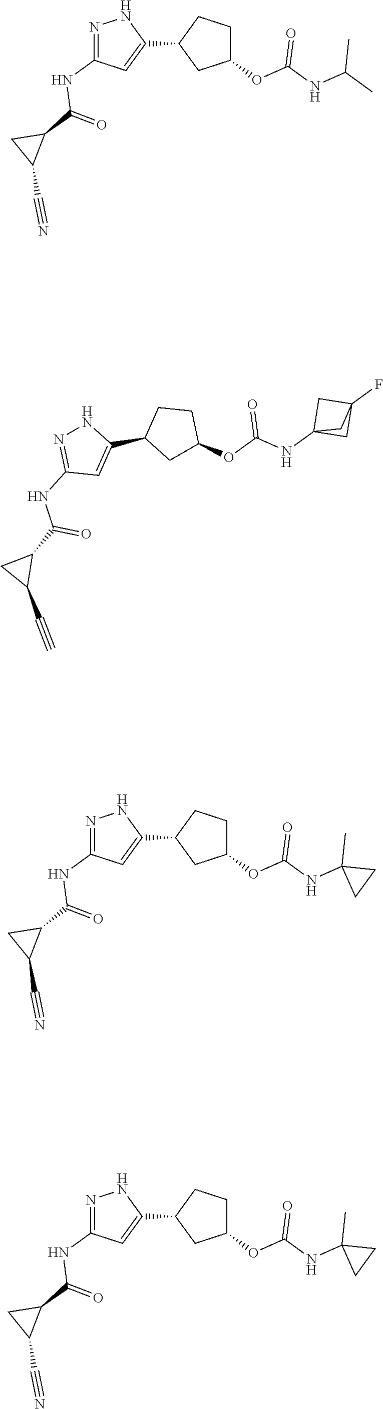 [SMILES: CC(C)NC(=O)O[C@H]1CC[C@@H](c2cc(NC(=O)[C@@H]3C[C@H]3C#N)n[nH]2)C1 ; C#C[C@H]1C[C@@H]1C(=O)Nc1cc([C@H]2CC[C@@H](OC(=O)NC34CC(F)(C3)C4)C2)[nH]n1 ; CC1(NC(=O)O[C@H]2CC[C@@H](c3cc(NC(=O)[C@H]4C[C@@H]4C#N)n[nH]3)C2)CC1 ; CC1(NC(=O)O[C@H]2CC[C@@H](c3cc(NC(=O)[C@@H]4C[C@H]4C#N)n[nH]3)C2)CC1]

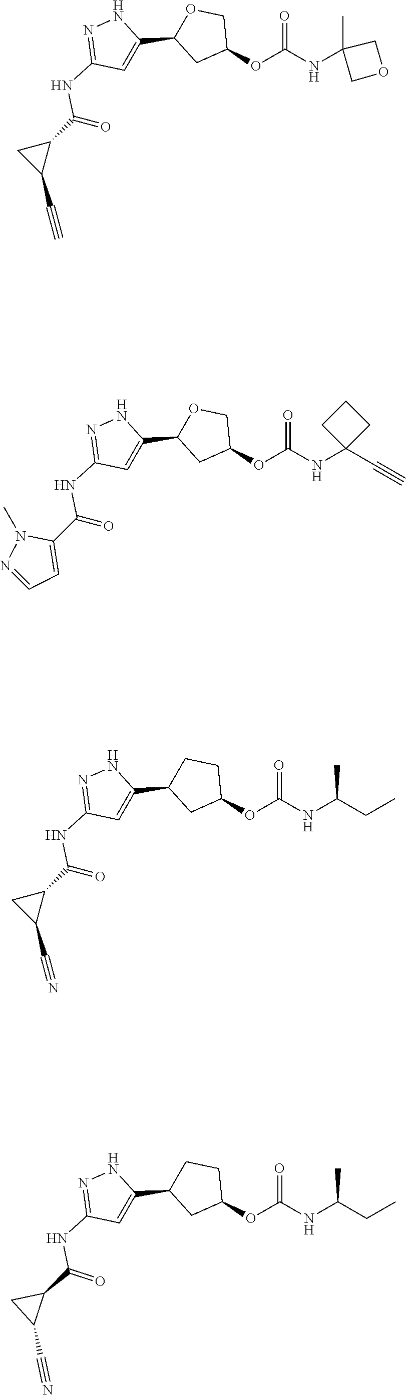 [SMILES: C#C[C@H]1C[C@@H]1C(=O)Nc1cc([C@@H]2C[C@H](OC(=O)NC3(C)COC3)CO2)[nH]n1 ; C#CC1(NC(=O)O[C@@H]2CO[C@H](c3cc(NC(=O)c4ccnn4C)n[nH]3)C2)CCC1 ; CC[C@H](C)NC(=O)O[C@@H]1CC[C@H](c2cc(NC(=O)[C@H]3C[C@@H]3C#N)n[nH]2)C1 ; CC[C@H](C)NC(=O)O[C@@H]1CC[C@H](c2cc(NC(=O)[C@@H]3C[C@H]3C#N)n[nH]2)C1]

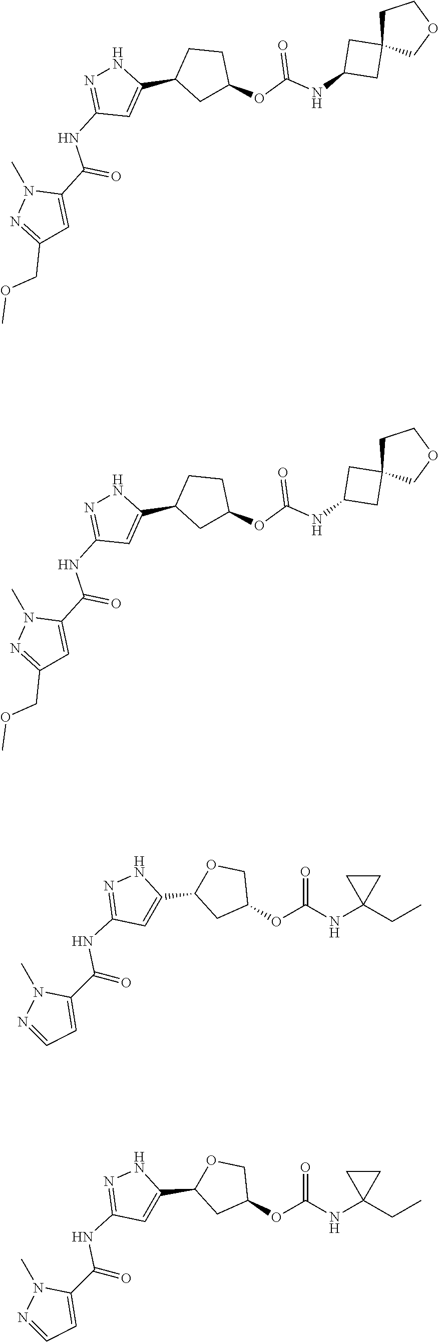

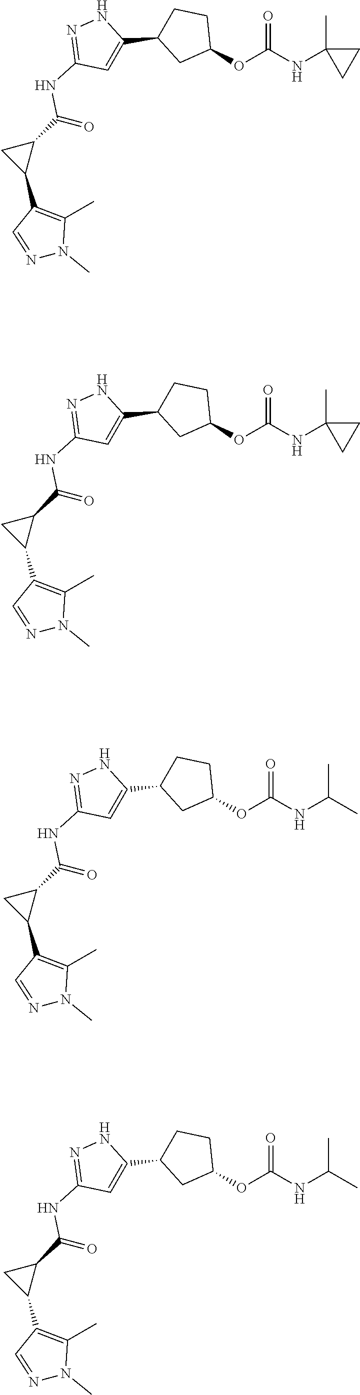 [SMILES: Cc1c([C@H]2C[C@@H]2C(=O)Nc2cc([C@H]3CC[C@@H](OC(=O)NC4(C)CC4)C3)[nH]n2)cnn1C ; Cc1c([C@@H]2C[C@H]2C(=O)Nc2cc([C@H]3CC[C@@H](OC(=O)NC4(C)CC4)C3)[nH]n2)cnn1C ; Cc1c([C@H]2C[C@@H]2C(=O)Nc2cc([C@@H]3CC[C@H](OC(=O)NC(C)C)C3)[nH]n2)cnn1C ; Cc1c([C@@H]2C[C@H]2C(=O)Nc2cc([C@@H]3CC[C@H](OC(=O)NC(C)C)C3)[nH]n2)cnn1C]

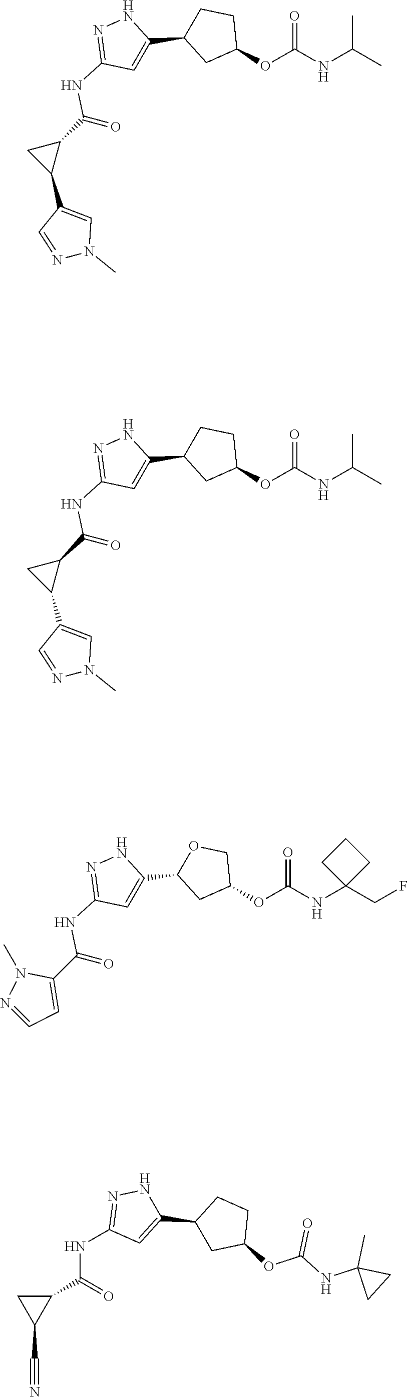

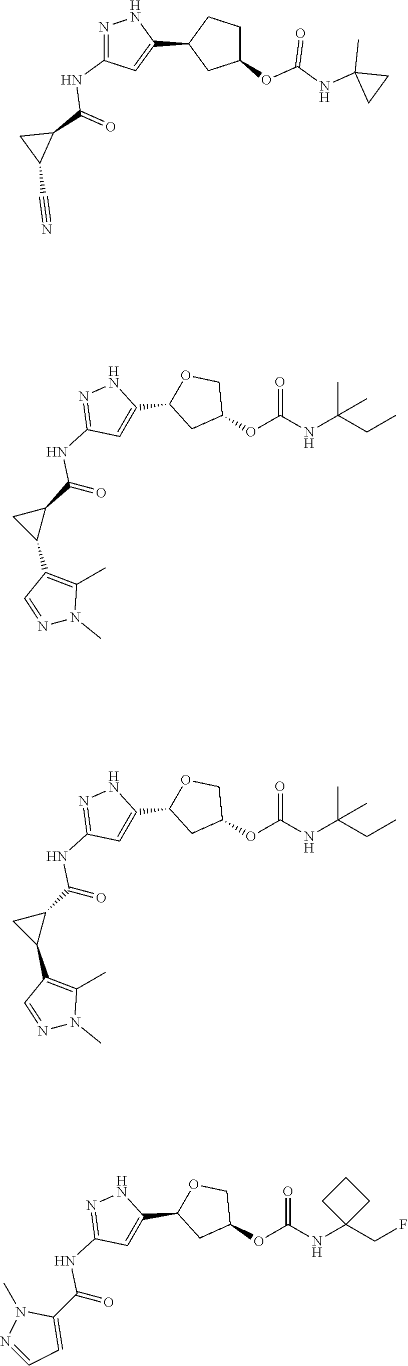

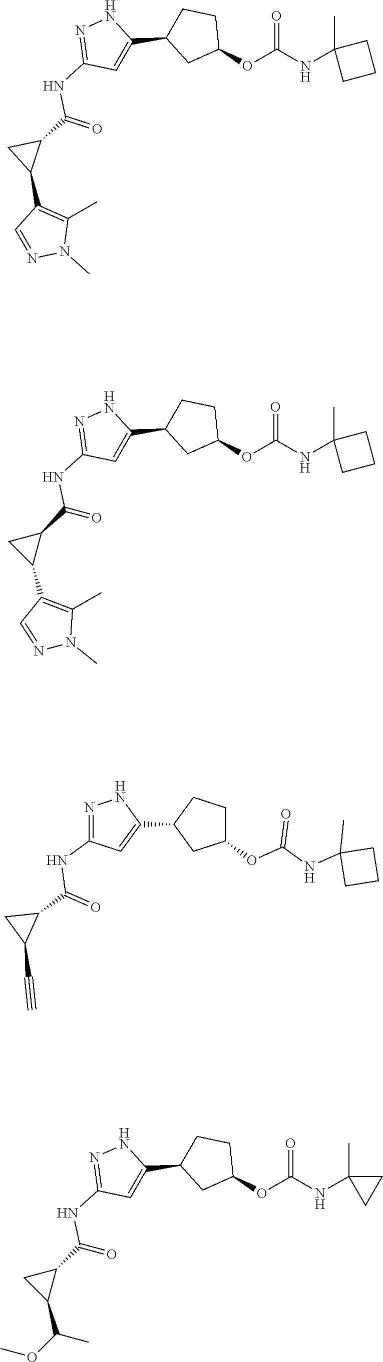 [SMILES: Cc1c([C@H]2C[C@@H]2C(=O)Nc2cc([C@H]3CC[C@@H](OC(=O)NC4(C)CCC4)C3)[nH]n2)cnn1C ; Cc1c([C@@H]2C[C@H]2C(=O)Nc2cc([C@H]3CC[C@@H](OC(=O)NC4(C)CCC4)C3)[nH]n2)cnn1C ; C#C[C@H]1C[C@@H]1C(=O)Nc1cc([C@@H]2CC[C@H](OC(=O)NC3(C)CCC3)C2)[nH]n1 ; COC(C)[C@H]1C[C@@H]1C(=O)Nc1cc([C@H]2CC[C@@H](OC(=O)NC3(C)CC3)C2)[nH]n1]

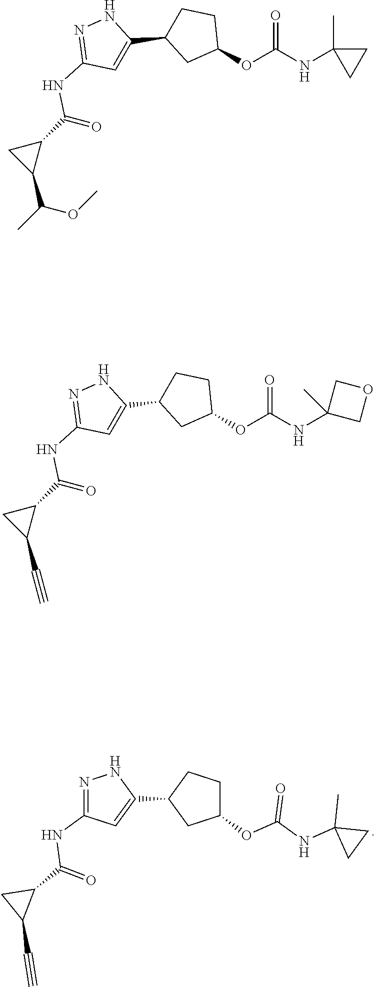 [SMILES: COC(C)[C@H]1C[C@@H]1C(=O)Nc1cc([C@H]2CC[C@@H](OC(=O)NC3(C)CC3)C2)[nH]n1 ; C#C[C@H]1C[C@@H]1C(=O)Nc1cc([C@@H]2CC[C@H](OC(=O)NC3(C)COC3)C2)[nH]n1 ; C#C[C@H]1C[C@@H]1C(=O)Nc1cc([C@@H]2CC[C@H](OC(=O)NC3(C)CC3)C2)[nH]n1]

In one embodiment, the present invention provides a preparation method of the compound, and the preparation method includes the following steps:

The compound represented by formula 1a or 1b can be prepared by the following scheme 1. 1a-2 can be synthesized from the raw material 1a-1 by activated carbonate displacement etc., then the protective group of the amino group (such as a Cbz group) on the pyrazole ring is removed to give 1a-3 which is coupled with a carboxylic acid to give 1a-4, finally the tert-butyl protecting group on the pyrazole ring is removed to obtain the final product.

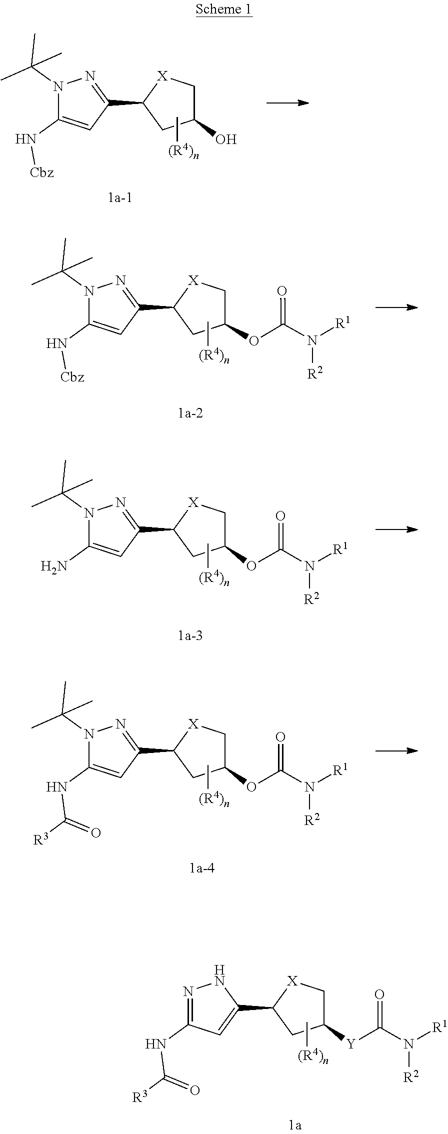

The compound of formula 1a or 1b can also be prepared by the following scheme 2. The alcoholic hydroxyl group is firstly transferred into an activated carbonate, then the tert-butyl protecting group on the pyrazole ring of 1a-5 is removed to obtain 1a-6, which is then reacted with an amine to get 1a-7, then the amino group on the pyrazole is protected, and then a procedure similar as that in scheme 1 is performed to synthesize the final product.

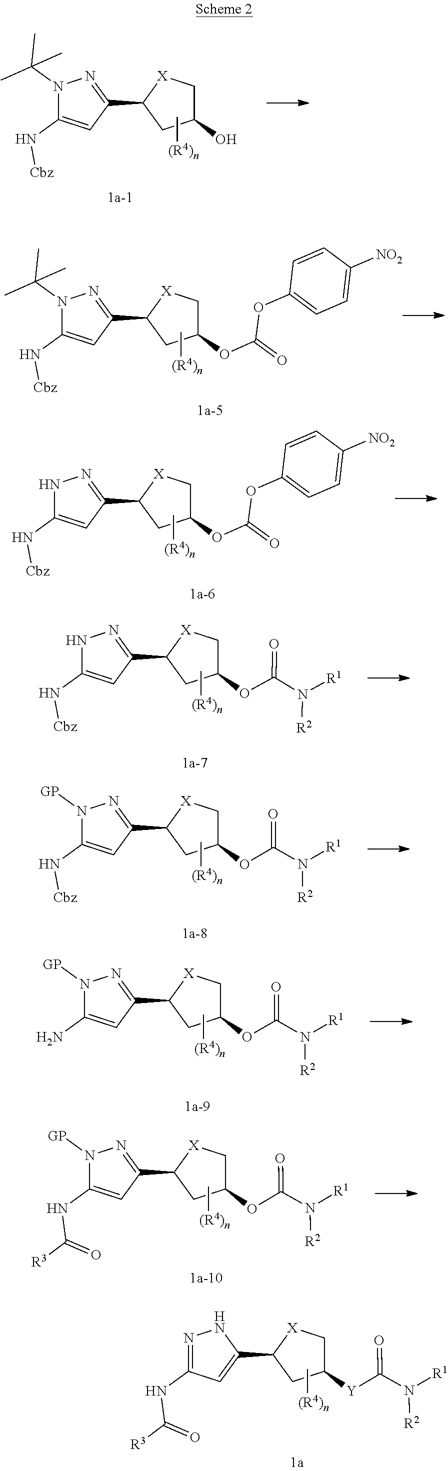

The compound of formula 1a or 1b can also be prepared by the following scheme 3. The hydroxyl group in 1a-1 is firstly protected, then the protecting group of the amino group on the pyrazole ring is removed, then 1a-14 is obtained by amide condensation and deprotection of the hydroxyl group. The final product can be obtained from deprotection of 1a-15 which is derived from the intermediate 1a-14 through carbamate formation, or alternatively from 1a-14 through active carbonate formation (1a-16), deprotection by removing the t-butyl on the pyrazole ring (1a-17), and displacement with an amine.

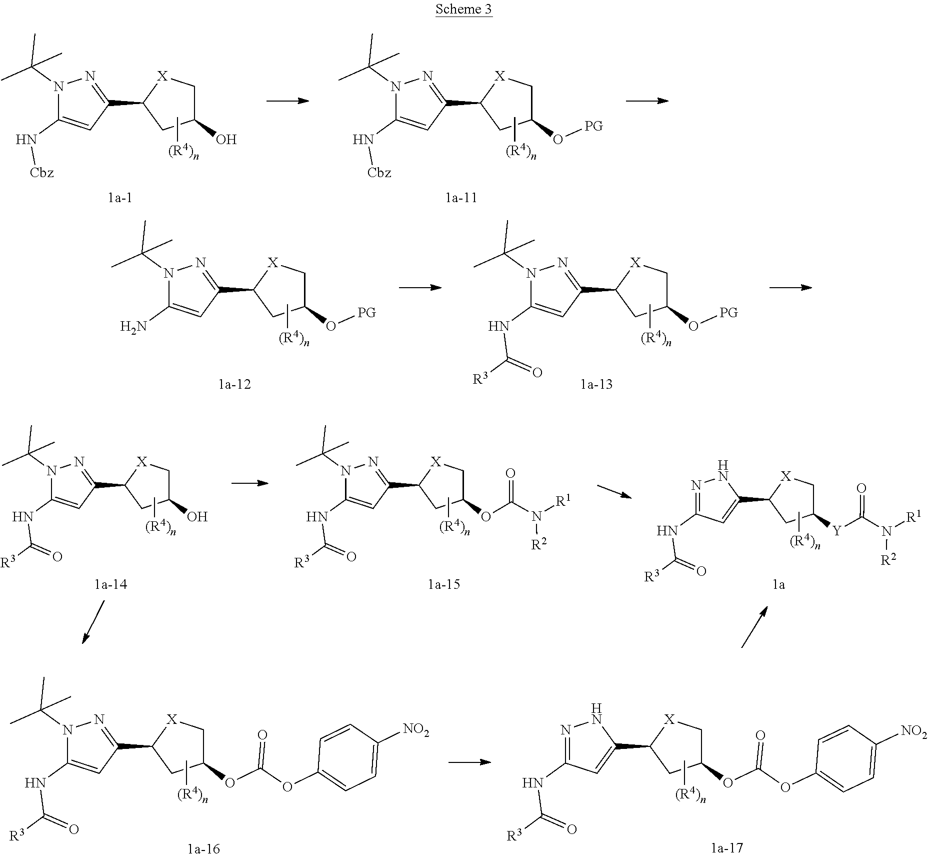

The compound of formula 1a or 1b can also be prepared by the following scheme 4. Similar to the procedure of scheme 1, 1a-19 can be prepared from 1a-18 firstly, then the protective group of the amino group attached to the pyrazole ring is removed to give 1a-20, and 1a-21 is obtained by condensation of an carboxylic acid with 1a-20, the enantiomeric pure final product is obtained by chiral separation after deprotection of racemic 1a-21 or chiral separation of racemic 1a-21 first followed by deprotection of the tert-butyl group.

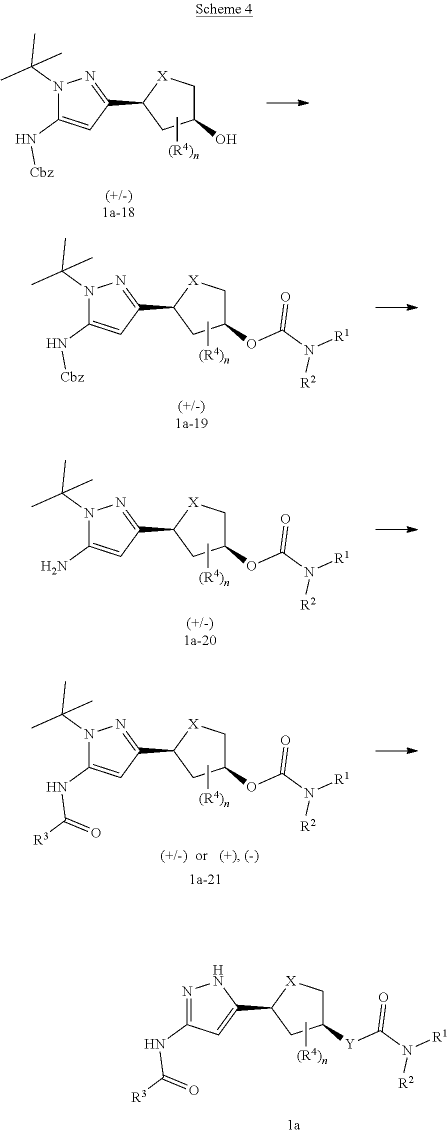

In one embodiment, the present invention provides a pharmaceutical composition comprising the compound, or an optical isomer or pharmaceutically acceptable salt thereof according to the present invention, and a pharmaceutically acceptable excipient or carrier.

In one embodiment, the present invention provides use of the compound, or an optical isomer or pharmaceutically acceptable salt thereof in preparation of a CDK2 inhibitor.

In one embodiment, the present invention provides use of the compound, or an optical isomer, prodrug or pharmaceutically acceptable salt thereof in preparation of a drug for treatment of an abnormal cell growth disease.

In one embodiment, the present invention provides a method for treating an abnormal cell growth disease, comprising administering to a subject in need thereof an effective amount of the compound, or an optical isomer or pharmaceutically acceptable salt thereof or the pharmaceutical composition according to the present invention.

In one embodiment, the abnormal cell growth disease is cancer.

In one embodiment, the cancer is selected from breast cancer, ovarian cancer, bladder cancer, uterine cancer, prostate cancer, lung cancer, esophageal cancer, head and neck cancer, colorectal cancer, kidney cancer (including renal cell carcinoma), liver cancer (including hepatocellular carcinoma), pancreatic cancer, gastric cancer or thyroid cancer.

In one embodiment, the lung cancer is selected from non-small cell lung cancer, small cell lung cancer, squamous cell carcinoma or adenocarcinoma.

### DETAILED DESCRIPTION

Hereinafter, the present invention will be described in detail. Before proceeding with the description, it should be understood that the terms used in the specification and the claims should not be construed as being limited to the general meaning and dictionary meaning, but should be explained according to the meaning and concept corresponding to the technical aspects of the present invention on the basis of the principle of allowing the inventor to properly define the terms for the best interpretation. Therefore, the description presented here is only a preferred example for illustrative purposes, and is not intended to limit the scope of the present invention. Therefore, it should be understood that various modifications and equivalents may be made without departing from the spirit or scope of the present invention,

When a range of values is listed, it is intended to encompass each value and sub-range within the range. For example “C1 to C8” is intended to encompass C₁, C₂, C₃, C₄, C₅, C₆, C₇, C₈, C₁₋₈, C₁₋₇, C₁₋₆, C₁₋₅, C₁₋₄, C₁₋₃, C₁₋₂, C₂₋₈, C₂₋₇, C₂₋₆, C₂₋₅, C₂₋₄, C₂₋₃, C₃₋₈, C₃₋₇, C₃₋₆, C₃₋₅, C₃₋₄, C₄₋₈, C₄₋₇, C₄₋₆, C₄₋₅, C₅₋₈, C₅₋₇, C₅₋₆, C₆₋₈, C₆₋₇ and C₇₋₈.

### Definition

“Alkyl” refers to a straight or branched chain saturated hydrocarbonyl having 1 to 8 carbon atoms (“C₁₋₈ alkyl”). In some embodiments, the alkyl has 1 to 7 carbon atoms (“C₁₋₇ alkyl”). In some embodiments, the alkyl has 1 to 6 carbon atoms (“C₁₋₆ alkyl”). In some embodiments, the alkyl has 1 to 5 carbon atoms (“C₁₋₅ alkyl”). In some embodiments, the alkyl has 1 to 4 carbon atoms (“C₁₋₄ alkyl”). In some embodiments, the alkyl has 1 to 3 carbon atoms (“C₁₋₃ alkyl”). In some embodiments, the alkyl has 1 to 2 carbon atoms (“C₁₋₂ alkyl”). In some embodiments, the alkyl group has 1 carbon atom (“C₁ alkyl”). In some embodiments, the alkyl has 1 to 6 carbon atoms (“C₁₋₆ alkyl”). Examples of C₁₋₆ alkyl include methyl (C₁), ethyl (C₂), propyl (C₃) (for example, n-propyl, isopropyl), butyl (C₄) (for example, n-butyl, tert-butyl, sec-butyl, isobutyl), pentyl (C₅) (for example, n-pentyl, 3-pentyl, neopentyl, 3-methyl-2-butyl, tert-pentyl) and hexyl (C₆) (for example, n-hexyl). Additional examples of alkyl include n-heptyl (C₇), n-octyl (C₈), and the like. Unless otherwise stated, each example of alkyl is independently unsubstituted (“unsubstituted alkyl”) or substituted with one or more substituents (e.g., halogen, such as F) (“substituted alkyl”). In some embodiments, the alkyl is an unsubstituted C₁₋₈ alkyl (e.g., unsubstituted C₁ alkyl, such as —CH₃). In some embodiments, the alkyl is a substituted C₁₋₈ alkyl (e.g., a substituted C₁ alkyl, such as —CF₃).

“Alkenyl” refers to a straight or branched hydrocarbonyl having 2 to 15 carbon atoms, one or more carbon-carbon double bonds and no triple bonds (“C₂₋₁₅ alkenyl”). In some embodiments, the alkenyl has 2 to 15 carbon atoms (“C₂₋₁₅ alkenyl”). In some embodiments, the alkenyl has 2 to 14 carbon atoms (“C₂₋₁₄ alkenyl”). In some embodiments, the alkenyl has 2 to 13 carbon atoms (“C₂₋₁₃ alkenyl”). In some embodiments, an alkenyl has 2 to 12 carbon atoms (“C₂₋₁₂ alkenyl”). In some embodiments, an alkenyl has 2 to 11 carbon atoms (“C₂₋₁₁ alkenyl”). In some embodiments, the alkenyl group has 2 to 10 carbon atoms (“C₂₋₁₀ alkenyl”). In some embodiments, the alkenyl has 2 to 9 carbon atoms (“C₂₋₉ alkenyl”). In some embodiments, the alkenyl has 2 to 8 carbon atoms (“C₂₋₈ alkenyl”). In some embodiments, the alkenyl has 2 to 7 carbon atoms (“C₂₋₇ alkenyl”). In some embodiments, the alkenyl group has 2 to 6 carbon atoms (“C₂₋₆ alkenyl”). In some embodiments, the alkenyl has 2 to 5 carbon atoms (“C₂₋₅ alkenyl”). In some embodiments, the alkenyl has 2 to 4 carbon atoms (“C₂₋₄ alkenyl”). In some embodiments, the alkenyl has 2 to 3 carbon atoms (“C₂₋₃ alkenyl”). In some embodiments, the alkenyl has 2 carbon atoms (“C₂ alkenyl”). The one or more carbon-carbon double bonds may be located in the middle (for example in 2-butenyl) or at the end (for example in 1-butenyl). Examples of C₂₋₄ alkenyl include vinyl (C₂), 1-propenyl (C₃), 2-propenyl (C₃), 1-butenyl (C₄), 2-butenyl (C₄), butadienyl (C₄), and the like. Examples of C₂₋₆ alkenyl include the aforementioned C₂₋₄ alkenyl as well as pentenyl (C₅), pentadienyl (C₅), hexenyl (C6), and the like. Additional examples of alkenyl include heptenyl (C7), octenyl (C₈), octatrienyl (C₈), and the like. Unless otherwise stated, each example of alkenyl is independently optionally substituted, that is, unsubstituted (“unsubstituted alkenyl”) or substituted with one or more substituents (“substituted alkenyl”). In some embodiments, the alkenyl is an unsubstituted C₂₋₁₀ alkenyl. In some embodiments, the alkenyl is a substituted C₂₋₁₀ alkenyl. In the alkenyl, a C═C double bond without specified stereochemistry (for example, —CH═CHCH₃ or

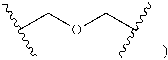 [SMILES: *COC*]

can be an (E)- or (Z)-double bond.

“Alkynyl” refers to a straight or branched hydrocarbonyl having 2 to 15 carbon atoms and one or more carbon-carbon triple bonds (“C₂₋₁₅ alkyne”). In some embodiments, the alkynyl has 2 to 15 carbon atoms (“C₂₋₁₅ alkynyl”). In some embodiments, the alkynyl has 2 to 14 carbon atoms (“C₂₋₁₄ alkynyl”). In some embodiments, the alkynyl has 2 to 13 carbon atoms (“C₂₋₁₃ alkynyl”). In some embodiments, the alkynyl has 2 to 12 carbon atoms (“C₂₋₁₂ alkynyl”). In some embodiments, the alkynyl has 2 to 11 carbon atoms (“C₂₋₁₁ alkynyl”). In some embodiments, the alkynyl has 2 to 10 carbon atoms (“C₂₋₁₀ alkynyl”). In some embodiments, the alkynyl has 2 to 9 carbon atoms (“C₂₋₉ alkynyl”). In some embodiments, the alkynyl has 2 to 8 carbon atoms (“C₂₋₈ alkynyl”). In some embodiments, the alkynyl has 2 to 7 carbon atoms (“C₂₋₇ alkynyl”). In some embodiments, the alkynyl has 2 to 6 carbon atoms (“C₂₋₆ alkynyl”). In some embodiments, the alkynyl has 2 to 5 carbon atoms (“C₂₋₅ alkynyl”). In some embodiments, the alkynyl has 2 to 4 carbon atoms (“C₂₋₄ alkynyl”). In some embodiments, the alkynyl has 2 to 3 carbon atoms (“C₂₋₃ alkynyl”). In some embodiments, the alkynyl has 2 carbon atoms (“C₂ alkynyl”). The one or more carbon-carbon triple bonds may be located in the middle (for example in 2-butynyl) or at the end (for example in 1-butynyl). Examples of C₂₋₄ alkynyl include, but are not limited to, ethynyl (C₂), 1-propynyl (C₃), 2-propynyl (C₃), 1-butynyl (C₄), 2-butynyl (C₄) and the like. Examples of C₂₋₆ alkynyl include the aforementioned C₂₋₄ alkynyl, as well as pentynyl (C₅), hexynyl (C₆), and the like. Additional examples of alkynyl include heptynyl (C₇), octynyl (C₈), and the like. Unless otherwise stated, each example of the alkynyl is independently optionally substituted, that is, unsubstituted (“unsubstituted alkynyl”) or substituted with one or more substituents (“substituted alkynyl”). In some embodiments, the alkynyl is an unsubstituted C₂₋₁₀ alkynyl. In some embodiments, the alkynyl is a substituted C₂₋₁₀ alkynyl.

“Alkoxy” means a monovalent —O-alkyl, where the alkyl moiety has the specified number of carbon atoms. In the present disclosure the alkoxy typically contains 1-15 carbon atoms (“C1 to C15 alkoxy”), or 1-8 carbon atoms (“C1-C8 alkoxy”) or 1-6 carbon atoms (“C1-C6 alkoxy”), or 1-4 carbon atoms (“C1-C4 alkoxy”). For example, C1-C4 alkoxy includes methoxy, ethoxy, isopropoxy, tert-butyloxy and the like. Unless otherwise stated, each example of the alkoxy is independently optionally substituted, that is, unsubstituted (“unsubstituted alkoxy”) or substituted with one or more substituents (“substituted alkoxy”). In some embodiments, the alkoxy is an unsubstituted C₁ to C15 alkoxy. In some embodiments, the alkoxy is a substituted C₁ to C15 alkoxy.

“Cycloalkyl” refers to a group of a non-aromatic cyclic hydrocarbonyl having 3 to 15 ring carbon atoms (“C₃₋₁₅ cycloalkyl”) and zero heteroatoms in the non-aromatic ring system. In some embodiments, the cycloalkyl has 3 to 10 ring carbon atoms (“C₃₋₁₀ cycloalkyl”). In some embodiments, the cycloalkyl has 3 to 6 ring carbon atoms (“C₃₋₆ cycloalkyl”). In some embodiments, the cycloalkyl has 5 to 10 ring carbon atoms (“C₅₋₁₀ cycloalkyl”). Exemplary C₃₋₆ cycloalkyls include, but are not limited to, cyclopropyl (C3), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, but are not limited to, the above-mentioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), and the like. Unless otherwise specified, each example of the cycloalkyl is independently optionally substituted, ie, unsubstituted (“unsubstituted cycloalkyl”) or substituted with one or more substituents (“substituted cycloalkyl”). In some embodiments, the cycloalkyl is an unsubstituted C₃₋₁₀ cycloalkyl. In some embodiments, the cycloalkyl is a substituted C₃₋₁₀ cycloalkyl. The cycloalkyl can be a monocyclic ring or a fused, bridged or spiro ring system, such as a bicyclic ring system, and can be saturated or partially unsaturated.

“Heterocycloalkyl” refers to a group of 3 to 15 membered non-aromatic ring system (“3-15 membered heterocyclyl”) having one or more ring carbon atoms and 1 to 4 ring heteroatoms, wherein each heteroatom is independently selected from nitrogen, oxygen, sulfur, boron, phosphorus and silicon. In a heterocyclyl containing one or more nitrogen atoms, the point of attachment may be a carbon atom or a nitrogen atom where the valence allows. The heterocyclyl can be a monocyclic ring (“monocyclic heterocyclyl”) or a fused, bridged or spiro ring system, such as a bicyclic ring system (“bicyclic heterocyclyl”), and can be saturated or partially unsaturated. A bicyclic ring system of the heterocyclyl may contain one or more heteroatoms in one or both rings. “Heterocyclyl” also includes a ring system in which a heterocyclic ring as defined above is fused with one or more cycloalkyl groups (wherein the point of attachment is on the cycloalkyl group or the heterocyclic ring), or in which a heterocyclic ring as defined above is fused with one or more aryl or heteroaryl groups (where the point of attachment is on the heterocyclic ring), and in this case, the number of ring members still refer to the number of the ring members in the heterocyclic ring system. Unless otherwise indicated, each example of the heterocyclyl is independently optionally substituted, that is, unsubstituted (“unsubstituted heterocycloalkyl”) or substituted with one or more substituents (“substituted heterocycloalkyl”). In some embodiments, the heterocycloalkyl is an unsubstituted 3-10 membered heterocycloalkyl. In some embodiments, the heterocycloalkyl is a substituted 3-10 membered heterocycloalkyl.

“Aryl” or “aromatic ring” or “aromatic cyclic group” refers to a group of a monocyclic or polycyclic (for example, bicyclic or tricyclic) 4n+2 aromatic ring system (e.g., shared with 6, 10, or 14 π electrons in the cyclic array) with 6-14 ring carbon atoms and zero heteroatoms in the aromatic ring system (“C₆₋₁₄ aryl”). In some embodiments, the aryl has 6 ring carbon atoms (“C₆ aryl”; e.g., phenyl). In some embodiments, the aryl has 10 ring carbon atoms (“C₁₀ aryl” e.g., naphthyl such as 1-naphthyl and 2-naphthyl). In some embodiments, the aryl has 14 ring carbon atoms (“C₁₄ aryl”, e.g., anthryl). The “aryl” also includes a ring system in which an aryl ring as defined above is fused with one or more cycloalkyl or heterocyclic groups (wherein the point of attachment is on the aryl ring), and in this case, the number of carbon atoms still refers to the number of carbon atoms in the aromatic ring system. Unless otherwise stated, each example of the aryl is independently optionally substituted, that is, unsubstituted (“unsubstituted aryl”) or substituted with one or more substituents (“substituted aryl”). In some embodiments, the aryl is an unsubstituted C₆₋₁₄ aryl. In some embodiments, the aryl is a substituted C₆₋₁₄ aryl.

“Aromatic heterocyclic ring” or “heteroaryl” refers to a group of a 5-10 membered monocyclic or bicyclic 4n+2 aromatic ring system (e.g., shared with 6, 10, or 14 π electrons in the cyclic array) with one or more ring carbon atoms and 1-4 ring heteroatoms in the aromatic ring system, wherein each heteroatom is independently selected from nitrogen, oxygen and sulfur (“5-10 membered heteroaryl”). In a heteroaryl containing one or more nitrogen atoms, the point of attachment may be a carbon atom or a nitrogen atom where the valence allows. A bicyclic ring system of the heteroaryl may contain one or more heteroatoms in one or both rings. The “heteroaryl” includes a ring system in which a heteroaryl ring as defined above is fused with one or more cycloalkyl or heterocyclic groups (wherein the point of attachment is on the heteroaryl ring), and in this case, the number of ring members still refers to the number of ring members in the heteroaryl ring system. The “heteroaryl” also includes a ring system in which a heteroaryl ring as defined above is fused with one or more aryl groups (where the point of attachment is on the aryl ring or the heteroaryl ring), and in this case, the number of ring members refers to the number of ring members in the fused (aryl/heteroaryl) ring system. In a bicyclic heteroaryl group where one ring does not contain a heteroatom (for example, indolyl, quinolinyl, carbazolyl, etc.), the point of attachment can be located on any of the rings, that is, on the ring with heteroatom(s) (for example, 2-indole group) or on the heteroatom-free ring (for example, 5-indolyl).

In some embodiments, the heteroaryl is a 5-10 membered aromatic ring system having one or more ring carbon atoms and 1-4 ring heteroatoms in the aromatic ring system, wherein each heteroatom is independently selected from nitrogen, oxygen and sulfur (“5-10 membered heteroaryl”). In some embodiments, the heteroaryl is a 5-8 membered aromatic ring system having one or more ring carbon atoms and 1-4 ring heteroatoms in the aromatic ring system, wherein each heteroatom is independently selected from nitrogen, oxygen and sulfur (“5-8 membered heteroaryl”). In some embodiments, the heteroaryl is a 5-6 membered aromatic ring system having one or more ring carbon atoms and 1-4 ring heteroatoms in the aromatic ring system, wherein each heteroatom is independently selected from nitrogen, oxygen and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has one ring heteroatom selected from nitrogen, oxygen, and sulfur. Unless otherwise specified, each example of the heteroaryl is independently optionally substituted, that is, unsubstituted (“unsubstituted heteroaryl”) or substituted with one or more substituents (“substituted heteroaryl”). In some embodiments, the heteroaryl is an unsubstituted 5-14 membered heteroaryl. In some embodiments, the heteroaryl is a substituted 5-14 membered heteroaryl.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, benzoisothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Fused ring” or “fused cyclic structure” refers to a 5-15 members polycyclic group wherein two or more monocyclic rings are fused by sharing a pair of bonding atoms, and all ring atoms are carbon atoms. Each monocyclic ring constituting the “fused ring” or “fused cyclic structure” may be an aromatic ring or a saturated or unsaturated aliphatic ring.

“Hetero fused ring” or “hetero fused ring structure” refers to a 5-15 membered polycyclic system wherein two or more monocyclic rings are fused by sharing a pair of bonding atoms, and one or more ring atoms are heteroatoms selected from nitrogen, oxygen, and sulfur and the remaining ring atoms are carbon atoms. Each monocyclic ring constituting the “hetero fused ring” or “hetero fused ring structure” may be an aromatic ring, an aromatic heterocyclic ring, a saturated or unsaturated aliphatic ring, or a saturated or unsaturated aliphatic heterocyclic ring.

“Spiro ring”, “aliphatic Spiro ring” or “Spiro ring structure” refers to a 4-15 membered polycyclic group, in which one atom (called Spiro atom) is shared between monocyclic rings and all ring atoms are carbon atoms. The “spiro ring” may be fused with one or more aromatic rings.

“Heterospiro ring”, “spiro heterocyclic ring” or “heterospiro ring structure” refers to a 4-15 membered poly-heterocyclic group, in which one atom (called spiro atom) is shared between monocyclic rings, and one or more ring atoms are heteroatoms selected from nitrogen, oxygen, and sulfur and the remaining ring atoms are carbon atoms.

“Bridged ring”, “aliphatic bridged ring” or “bridged ring structure” refers to a 5 to 15 membered polycyclic group in which any two rings share two atoms that are not directly connected, wherein these rings may contain one or more double bonds, but none of the rings have a fully conjugated p electron system, and all ring atoms are carbon atoms. According to the number of rings, it can be divided into bicyclic, tricyclic, tetracyclic or polycyclic bridged ring.

“Hetero bridged ring”, “bridged heterocyclic ring” or “hetero-bridged ring structure” refers to a 5 to 15 membered poly-heterocyclic group in which any two rings share two atoms that are not directly connected, wherein these rings may contain one or more double bonds, but none of the rings have a fully conjugated p electron system, and one or more ring atoms are heteroatoms selected from nitrogen, oxygen, and sulfur, and the remaining ring atoms are carbon atoms. According to the number of rings, it can be divided into bicyclic, tricyclic, tetracyclic or polycyclic bridged heterocyclic ring.

“Halogen” or “halo” refers to fluorine (fluo, —F), chlorine (chlo, —Cl), bromine (bromo, —Br) or iodine (iodo, —I).

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Each example of R^{aa} is independently selected from C₁₋₁₀ alkyl, C₁₋₁₀ perhaloalkyl, C₂₋₁₀ alkenyl, C₂₋₁₀ alkynyl, C₃₋₁₀ cycloalkyl, 3-14 membered heterocyclyl, C₆₋₁₄ aryl and 5-14 membered heteroaryl, or two R^{aa} are connected to form a 3-14 membered heterocyclyl or a 5-14 membered heteroaryl ring.

Each example of R^{bb} is independently selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, C₃₋₁₀ cycloalkyl, 3-14 membered heterocyclyl, C₆₋₁₄ aryl and 5-14 membered heteroaryl, or two R^{bb} groups are connected to form a 3-14 membered heterocyclyl or a 5-14 membered heteroaryl ring.

Each example of R^{cc} is independently selected from hydrogen, C₁₋₁₀ alkyl, C₁₋₁₀ perhaloalkyl, C₂₋₁₀ alkenyl, C₂₋₁₀ alkynyl, C₃₋₁₀ cycloalkyl, 3-14 membered heterocyclyl, C₆₋₁₄ aryl and 5-14 membered heteroaryl, or two R^{cc} groups are connected to form a 3-14 membered heterocyclyl or a 5-14 membered heteroaryl ring.

“Substituted” or “optionally substituted” means that an atom, such as a hydrogen atom, in the group is substituted. In certain embodiments, alkyl, alkenyl, alkynyl, carbocyclyl, heterocyclyl, aryl, and heteroaryl are substituted (e.g., “substituted” alkyl, “substituted” alkenyl, “substituted” alkynyl, “substituted” carbocyclyl, “substituted” heterocyclyl, “substituted” aryl or “substituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group may have one or more substituents at one or more substitutable positions of the group, and when more than one position in any given structure is substituted, the substituents are the same or different at each position. By the term “substituted”, it is intended to include substitution with all permissible substituents of organic compounds, any of the substituents described herein that results in the formation of a stable compound. The present disclosure contemplates any and all such combinations to arrive at stable compounds. For purposes of this disclosure, a heteroatom such as nitrogen may have a hydrogen substituent and/or any suitable substituent as described herein which satisfies the valence of the heteroatom and results in the formation of a stable moiety. In certain embodiments, the substituent is a substituent on a carbon atom. In certain embodiments, the substituent is a substituent on a nitrogen atom. In certain embodiments, the substituent is a substituent on an oxygen atom. In certain embodiments, the substituent is a substituent on a sulfur atom.

“Unsaturated” or “partially unsaturated” refers to a group containing at least one double or triple bond. The “partially unsaturated” ring system is also intended to encompass a ring with multiple unsaturated sites, but is not intended to include aromatic groups (e.g., aryl or heteroaryl). Similarly, “saturated” refers to a group that does not contain a double or triple bond, that is, the group only contains single bonds.

The term “pharmaceutically acceptable” used herein is intended to refer to those compounds, materials, compositions and/or dosage forms which, within the scope of reliable medical judgment, are suitable for use in contact with human and animal tissues without excessive toxic, irritant, allergic response, or other problems or complications, and commensurate with a reasonable benefit/risk ratio.

The term “pharmaceutically acceptable salt” refers to a salt of the compound of the present invention, which is prepared from a compound with specific substituents discovered in the present invention and a relatively non-toxic acid or base. When the compound of the present invention contains a relatively acidic functional group, a base addition salt can be obtained by contacting the neutral form of the compound with a sufficient amount of a base in a pure solution or a suitable inert solvent. Pharmaceutically acceptable base addition salts include sodium, potassium, calcium, ammonium, organic ammonia or magnesium salts or similar salts. When the compound of the present invention contains a relatively basic functional group, an acid addition salt (i.e., a pharmaceutically acceptable salt) can be obtained by contacting the neutral form of the compound with a sufficient amount of an acid in a pure solution or a suitable inert solvent, and the examples thereof include inorganic acid salts and organic acid salts. The inorganic acid includes, for example, hydrochloric acid, hydrobromic acid, nitric acid, carbonic acid, hydrogen carbonate, phosphoric acid, monohydrogen phosphate, dihydrogen phosphate, sulfuric acid, monohydrogen sulfate, hydroiodic acid, phosphorous acid, etc.; the organic acid includes, for example, benzoic acid, 2-hydroxyethanesulfonic acid, sulfamic acid, benzenesulfonic acid, phenylacetic acid, mandelic acid, malonic acid, propionic acid, oxalic acid, p-aminobenzenesulfonic acid, p-toluenesulfonic acid, polygalacturonic acid, pantothenic acid, fumaric acid, glutamic acid, succinic acid, methanesulfonic acid, tartaric acid, ascorbic acid, phthalic acid, maleic acid, citric acid, malic acid, glucoheptonic acid, gluconic acid, isethionic acid, lactic acid, lactobionic acid, dodecylsulfonic acid, pamoic acid, salicylic acid, suberic acid, folinic acid, edetic acid, glycolic acid, acetic acid, ethanesulfonic acid, isobutyric acid, stearic acid and other similar acids; also included are salts of amino acids (such as arginine, etc.), and salts of organic acids such as glucuronic acid (referring to Berge et al., “Pharmaceutical Salts”, Journal of Pharmaceutical Science 66: 1-19 (1977)). Some specific compounds of the present invention contain basic and acidic functional groups, which can be converted into any base or acid addition salt. The parent forms and various salt forms of the compounds differ in some physical properties, such as solubility in polar solvents.

As used herein, the term “about” refers to variation in the numerical quantity that may occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

As used herein, the terms “treat”, “treating”, “treatment” and the like refer to eliminating, reducing, or ameliorating a disease or condition, and/or symptoms associated therewith. Although not precluded, treating a disease or condition does not require complete elimination of the disease, condition, or symptoms associated therewith. As used herein, the terms “treat”, “treating”, “treatment” and the like may include “prophylactic treatment” which refers to reducing the possibility of redevelopment of a disease or condition or recurrence of a disease or condition previously controlled in a subject who is not or at risk of suffering from or susceptible to a disease or condition or recurrence of a disease or condition. The term “treat” and synonyms thereof contemplate administering a therapeutically effective amount of the compound of the present disclosure to a subject in need of such treatment.

With regard to a drug or a pharmacologically active agent, the term “effective amount” or “therapeutically effective amount” refers to a sufficient amount of the drug or agent that is non-toxic but can achieve the desired effect. For the oral dosage form of the present invention, the “effective amount” of one active substance in the composition refers to the amount required to achieve the desired effect when combined with another active substance in the composition. The determination of the effective amount varies from different subjects, and depends on the age and general conditions of the subject, as well as the specific active substance. The appropriate effective amount in a specific case can be determined by those skilled in the art according to routine experiments.

The term “subject”, “patient” or “object” refers to an animal that has become the object of treatment, observation or experiment, preferably a mammal, and most preferably a human. In any of the embodiments described herein, the subject can be a human.

The pharmaceutical composition described in the present disclosure can be prepared by any method known in the field of pharmacology. Generally, such preparation method includes mixing the active ingredient, such as the salt of the present invention, with a carrier or excipient and/or if necessary and/or desired, one or more other auxiliary ingredients, and then shaping and/or paking the product into the desirable single-dose or multiple-dose units.

The relative amounts of the active ingredients, pharmaceutically acceptable excipients and/or any other ingredients in the pharmaceutical composition described in the present disclosure can vary, depending on the identity, size and/or condition of the subject to be treated, and further on the route by which the composition will be administered. The composition may contain 0.1% to 100% (w/w) of the active ingredient.

The term “pharmaceutically acceptable excipient or carrier” refers to any preparation or carrier medium that can deliver an effective amount of the active substance of the present invention, does not interfere with the biological activity of the active substance, and has no toxic side effects to the host or patient. The representative carriers include water, oil, vegetables and minerals, cream substrates, lotion substrates, ointment substrates, etc. These substrates include suspending agents, tackifiers, penetration enhancers and the like. The pharmaceutically acceptable excipients that can be used to manufacture the pharmaceutical compositions described herein include, but are not limited to, for example, inert diluents, dispersing agents and/or granulating agents, surfactants and/or emulsifiers, disintegrating agents, adhesives, preservatives, buffers, lubricants and/or oils. Excipients, such as cocoa butter and suppository waxes, coloring agents, coating agents, sweetening agents, flavoring agents and perfuming agents may also be present in the composition.

The pharmaceutical composition can be formulated for any route of administration, such as oral administration. Generally, the pharmaceutical composition is in a solid form. However, in some embodiments, other dosage forms may also be used, such as liquid, suspension, or semi-solid dosage forms.

Solid dosage forms for oral administration include capsules, tablets, pills, powders, and granules. In such solid dosage forms, the active ingredient is mixed with at least one inert, pharmaceutically acceptable excipient or carrier (such as sodium citrate or dicalcium phosphate) and/or (a) a filler or extender (such as starch, lactose, sucrose, glucose, mannitol, and silicic acid), (b) a binder (such as carboxymethylcellulose, alginate, gelatin, polyvinylpyrrolidinone, sucrose, and arabic gum), (c) a humectant (such as glycerol), (d) a disintegrant (such as agar, calcium carbonate, potato or tapioca starch, alginic acid, certain silicates, and sodium carbonate), (e) a retardant (such as paraffin), (f) an absorption promoter (such as a quaternary ammonium compound), (g) a wetting agent such as, cetyl alcohol and glycerol monostearate, (h) an absorbent (such as kaolin and bentonite), and (i) a lubricant (such as talc, calcium stearate, magnesium stearate, solid polyethylene glycols, sodium lauryl sulfate), and a mixture thereof. In the case of capsules, tablets, and pills, these dosage forms may include a buffering agent.

The active ingredient (e.g., the compound of the present invention) may be in the form of a microcapsule with one or more excipients as described above. The solid dosage forms of tablets, dragees, capsules, pills and granules can be prepared with coatings and shells well known in the field of pharmaceutical preparation, such as enteric coatings, controlled release coatings and other coatings. In these solid dosage forms, the active ingredient can be mixed with at least one inert diluent such as sucrose, lactose or starch. Such dosage forms may contain substances other than inert diluents according to conventional practice, for example, a tableting lubricant and other tableting aids, such as magnesium stearate and microcrystalline cellulose. In the case of capsules, tablets and pills, these dosage forms may contain a buffering agent. They may optionally contain a sunscreen agent, and may have a composition that releases the active ingredients only or preferably in certain parts of the intestinal tract, optionally in a delayed manner. Examples of encapsulants that can be used include polymeric substances and waxes.

Although the description of the pharmaceutical composition provided in the present disclosure is mainly directed to a pharmaceutical composition suitable for administration to humans, such a composition is generally suitable for administration to various animals. In order to adapt the composition suitable for administration to various animals, modifications of the pharmaceutical composition suitable for administration to humans are well known, and a veterinary pharmacist of ordinary skill can design and/or perform such modifications through ordinary experiments. For veterinary use, according to normal veterinary practice, the compound of the present disclosure can be administered as an appropriately acceptable formulation. A veterinarian can easily determine the dosage regimen and route of administration most suitable for a particular animal.

The pharmaceutical composition of the present disclosure may be prepared, packaged, and/or sold in bulk as a single unit dose and/or as a plurality of single unit doses. A “unit dose” means a discrete amount of a pharmaceutical composition containing a predetermined amount of an active ingredient. The amount of the active ingredient is usually equal to the dose of the active ingredient to be administered to the subject and/or a convenient dose of the dose, such as half or one third of the dose. However, it should be understood that the total daily dosage of the composition described herein will be determined by the physician within the scope of reasonable medical judgment. For any specific subject or organism, the specific therapeutically effective dose level will depend on many factors, including the disease being treated and the severity of the disease; the activity of the used specific active ingredient; the used specific composition; the age, weight, general health, gender and diet of the subject; time of administration, route of administration and excretion rate of the specific active ingredient; duration of treatment; drugs used in combination or concurrently with the specific active ingredient; and factors well known in the medical field.

Various dosage forms of the pharmaceutical composition of the present disclosure can be prepared according to conventional preparation methods in the pharmaceutical field. The unit dose of the formulation may contain 0.05-200 mg, preferably 1 mg-100 mg of the compound.

The compound and pharmaceutical composition of the present disclosure can be used clinically in mammals, including humans and animals, and can be administered via oral, nose, skin, lung, or gastrointestinal tract. The best preferred daily dose is 0.1-20 mg/kg body weight, administered once or in divided doses. Regardless of the method of administration, the optimal dose for the patient should be determined based on the specific treatment. Clinical trials usually start with a small dose and gradually increase the dose until the most suitable dose is found.

In some embodiments, all the necessary components for treating a CDK2-related disease or condition comprising the compound of the present disclosure alone or in combination with another agent or intervention traditionally used to treat such disease can be packaged in a medical kit. Specifically, in some embodiments, the present invention provides a medical kit for treatment and intervention of a disease, which includes a set of packaged medicines, and the packaged medicines include the compound disclosed herein and a buffer and other components for preparing a deliverable form of the medicines, and/or devices used to deliver such medicines and/or any agent used in the combination therapy with the compound of the present disclosure, and/or instructions for the treating diseases packaged together with the medicines.

In some embodiments, the compound, or the optical isomer, prodrug, or pharmaceutically acceptable salt thereof according to the present invention has a higher selectivity for CDK2 than other CDKs, especially CDK1. In some embodiments, the compound, or the optical isomer, prodrug, or pharmaceutically acceptable salt thereof according to the present invention has a higher selectivity for CDK2 than CDK4 and/or CDK6. In other aspects and embodiments, the compound, or the optical isomer, prodrug or pharmaceutically acceptable salt thereof according to the present invention has a higher selectivity for CDK2 than glycogen synthase kinase 3β (GSK3β).

Based on experimental data, the inventors of the present invention believe that, compared with known CDK2 inhibitors, the compounds disclosed herein have better selectivity for CDK2 than for CDK1 with a stronger cell activity. These compounds also have better overall PK characteristics.

In some embodiments, further provided are treatment methods and uses, including administering the compound of the present invention, or the optical isomer, prodrug, or pharmaceutically acceptable salt thereof according to the present invention alone or in combination with another therapeutic agent or a buffer.

In some embodiments, the present invention provides a method for treating abnormal cell growth in a subject in need thereof, comprising administering to the subject a therapeutically effective amount of the compound, or the optical isomer, prodrug or pharmaceutically acceptable salt thereof according to the present invention. Also included is that an amount of the compound, or the optical isomer, prodrug, or pharmaceutically acceptable salt thereof according to the invention is administered to a subject in combination with an amount of an additional therapeutic agent (e.g., an anti-cancer therapeutic agent) to effectively treat the abnormal cell growth.

The abnormal cell growth comprises the following abnormal growth: (1) tumor cells (tumors) exhibiting increased CDK2 expression; (2) tumors proliferating through abnormal CDK2 activation; (3) tumors characterized in amplification or overexpression of CCNE1 and/or CCNE2 and (4) tumors resistant to endocrine therapy, HER2 antagonists, or CDK4/6 inhibition.

In common embodiments of the treatment methods provided by the present disclosure, the abnormal cell growth is cancer. The compound of the present invention may be administered as a single agent, or in combination with another anti-cancer therapeutic agent, especially a standard-of-care agent applicable to a specific cancer.

In some embodiments, the provided method generates one or more of the following effects: (1) inhibiting proliferation of tumor cells; (2) inhibiting invasion of tumor cells; (3) inducing apoptosis of tumor cells; (4) inducing metastasis of tumor cells; or (5) inhibiting angiogenesis.

In some embodiments, the present invention provides a method for treating a disorder mediated by CDK2 in a subject, comprising administering to the subject (especially a cancer patient) the compound or a pharmaceutically acceptable salt thereof according to the present invention in amount of effectively treating the disorder.

In some embodiments, the cancer is selected from breast cancer, ovarian cancer, bladder cancer, uterine cancer, prostate cancer, lung cancer (including non-small cell lung cancer, small cell lung cancer, squamous cell carcinoma or adenocarcinoma), esophageal cancer, head and neck cancer, colorectal cancer, kidney cancer (including renal cell carcinoma), liver cancer (including hepatocellular carcinoma), pancreatic cancer, gastric cancer or thyroid cancer.

The term “additional anti-cancer therapeutic agent” used in the present disclosure refers to any one or more therapeutic agents, which is used to or able to treat cancer, other than the compound, or the optical isomer, prodrug or pharmaceutically acceptable salt thereof according to the present invention. In some embodiments, such additional anticancer therapeutic agents include compounds derived from the following categories: mitotic inhibitors, alkylating agents, antimetabolites, antitumor antibiotics, antiangiogenic agents, topoisomerase I and II inhibitors, plant alkaloids, hormones and antagonists, growth factor inhibitors, radiation, signal transduction inhibitors such as protein tyrosine kinases and/or serine/threonine kinase inhibitors, cell cycle inhibitors, biological response modifiers, enzyme inhibitors, antisense oligonucleotides or oligonucleotide derivatives, cytotoxins, antitumor immunomodulators, and the like. In some embodiments, the additional anticancer agent is an endocrine agent, such as an aromatase inhibitor, a selective estrogen receptor degrading agent (SERD) or a selective estrogen receptor modulator (SERM). In other embodiments, the additional anti-cancer agent is a so-called classic anti-tumor agent. In some other embodiments, the additional anticancer agent is a genetic modulator, such as EZH2, SMARCA4, PBRM1, ARID1A, ARID2, ARID1B, etc.

In some embodiments, provided are any methods capable of delivering a compound to an acting site so as to realize the administration of the compound of the present invention. These methods include oral, intraduodenal, parenteral (including intravenous, subcutaneous, intramuscular, intravascular or infusion), topical and rectal administration.

The dosage regimen can be adjusted to provide optimal desired response. For example, the administration can be done by a single large dose delivery, or by several divided doses over time, or by proportionally reduced or increased dosage as dictated by the urgency of the treatment situation.

Suitable pharmaceutical carriers include inert diluents or fillers, water, and various organic solvents (such as hydrates and solvates). If necessary, the pharmaceutical composition may contain additional ingredients such as a flavoring agent, a binder, an excipient, and the like.

The pharmaceutical composition may, for example, be in a form suitable for oral administration, such as a tablet, a capsule, a pill, powder, a sustained-release preparation, a solution, a suspension; in a form suitable for parenteral injection, such as a sterile solution, a suspension or an emulsion; in a form suitable for topical application, such as an ointment or a cream; or in a form suitable for rectal application, such as a suppository.

The present invention also includes isotopically-labeled compounds of the present invention, and these isotopically-labeled compounds are identical to those recited herein, except that one or more atoms are replaced with atoms with an atomic mass or mass number different from those usually found in nature. Examples of isotopes that can be incorporated into the compounds of the invention include isotopes of hydrogen, carbon, nitrogen, oxygen, fluorine and chloride, such as ²H, ³H, ¹³C, ¹⁴C, ¹⁵N, ¹⁸O, ¹⁷O, ¹⁸F and ³⁶Cl.

Certain isotopically-labeled compounds of the present invention (for example, compounds labeled with ³H and ¹⁴C) can be used in identification of the compounds and/or substrate tissue distribution. Tritium (i.e., ³H) and carbon-14 (i.e., ¹⁴C) isotopes are particularly preferred for their ease of preparation and detection. Further, the compound substituted with a heavy isotope such as deuterium (i.e., ²H) may produce certain therapeutic advantages resulting from higher metabolic stability (for example, increased half-life in vivo or reduced dosage), and thus is preferably used in certain conditions. The isotopically-labeled compounds of the present invention may generally be prepared by following the procedures similar to those disclosed in the flow routes and/or examples below by replacing non-isotope labeled agents with isotopically-labeled agents.

The following examples are only listed as examples of the embodiments of the present invention, and do not constitute any limitation to the present invention. Those skilled in the art can understand that modifications within the scope that do not deviate from the nature and concept of the present invention fall into the protection scope of the present invention. Unless otherwise specified, the reagents and instruments used in the following examples are commercially available.

### Synthetic Methods

Compounds of the invention are prepared according to the exemplary procedures provided herein and modifications thereof known to those of skill in the art. The following abbreviations are used throughout the Examples: “Ac” means acetyl, “OAc” means acetoxy, “aq” means aqueous, “BOC”, “Boc” or “boc” means N-tert-butoxycarbonyl, “Bn” means benzyl, “Bu” means butyl, “n-Bu” means normal-butyl, “t-Bu” means tert-butyl, “Cbz” means benzyloxycarbonyl, “CMPI” means 2-chloro-1-methylpyridinium iodide, “DAST” means diethylaminosulphur trifluoride, “DCM” (CH₂Cl₂) means methylene chloride/dichloromethane, “de” means diastereomeric excess, “DEA” means diethylamine, “DIEA” means N-ethyl-N-isopropylpropan-2-amine, “DMA” means N,N-dimethylacetamide, “DMAP” means 4-dimethylaminopyridine, “DMF” means N,N-dimethyl formamide, “DMSO” means dimethylsulfoxide, “DPPA” means diphenyl azidophosphate, “ee” means enantiomeric excess, “Et” means ethyl, “EtOAc” means ethyl acetate, “EtOH” means ethanol, “FA” means formic acid, “HATU” means 1-[bis(dimethylamino)methylene]-1H-1,2,3-triazolo[4,5-b]pyridinium 3-oxid hexafluorophosphate, “HOAc” means acetic acid, “HPLC” means high performance liquid chromatography, “i-Pr” or “′Pr” means isopropyl, “IPA” means isopropyl alcohol, “LCMS” means liquid chromatography mass spectrometry, “Me” means methyl, “MeOH” means methanol, “MS” means mass spectrometry, “MsCl” means methanesulfonyl chloride, “MTBE” means methyl tert-butyl ether, “Ph” means phenyl, “sat.” means saturated, “SFC” means supercritical fluid chromatography, “TBAF” means tetrabutylammonium fluoride, “TBDPSCl” means tert-butylchlorodiphenylsilane, “TEA” means triethylamine, “TFA” means trifluoroacetic acid, “THF” means tetrahydrofuran, “TLC” means thin layer chromatography, “min” means minutes.

### PREPARATION OF INTERMEDIATES

### Intermediates: A1, A2

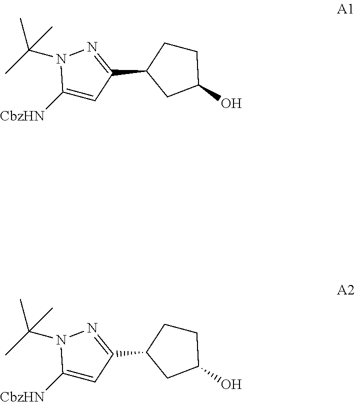 [SMILES: CC(C)(C)n1nc([C@H]2CC[C@@H](O)C2)cc1NC(=O)OCc1ccccc1 ; CC(C)(C)n1nc([C@@H]2CC[C@H](O)C2)cc1NC(=O)OCc1ccccc1]

### Synthetic Route of Intermediates A1, A2 as Follows (Route 1):

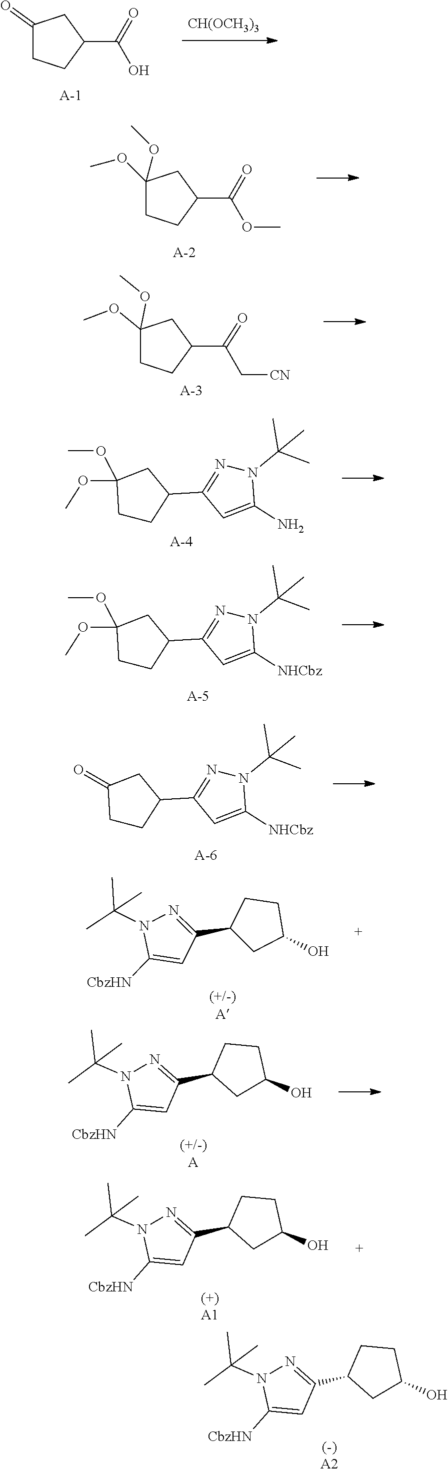

### Step 1 (Synthesis of Compound A-2)

To a solution of A-1 (100 g, 780.6 mmol, 1.0 eq) in methanol (560 mL) at room temperature were added with trimethyl orthoformate (497.3 g, 4.7 mol, 6 eq) and p-toluenesulfonic acid monohydrate (3.0 g, 15.6 mmol, 0.02 eq). The mixture was stirred at room temperature for 15 hours. TLC showed completion of A-1. The mixture was quenched with saturated aqueous NaHCO₃ solution and concentrated under vacuum to remove most of methanol. The residue was diluted with EtOAc (×3). The combined organic layers were washed with brine, dried over Na₂SO₄, filtered and concentrated to give A-2 (260.0 g).

### Step 2 (Synthesis of Compound A-3)

A solution of n-BuLi (212.5 mL of 2.5 M solution in hexanes, 531.3 mmol, 2.0 eq) was added into THE (475 mL) dropwise at −65° C. To the above solution was added anhydrous acetonitrile (27.8 mL, 531.3 mmol, 2.0 eq) dropwise slowly keeping the internal temperature below −55° C. The mixture was stirred for additional 1 hour at −65° C. A solution of A-2 (50 g, 265.7 mmol, 1.0 eq) in THE (55 mL) was then added dropwise slowly enough to maintain the internal temperature below −50° C. After addition, the reaction was stirred for 2 hours at −65° C., TLC showed completion of A-2. Five batches were carried out. The reaction was quenched with H₂O, neutralized with aqueous HCl solution (1 M) to pH 7, and extracted with EtOAc (×3). The combined organic layers of five batches were washed with brine, dried over Na₂SO₄, filtered and concentrated to give A-3 (160.0 g), which was used in next step without further purification.

### Step 3 (Synthesis of Compound A-4)

t-BuOK (34.1 g, 304.2 mmol, 1.5 eq) was added in portions to a suspension of tert-butylhydrazine hydrochloride (30.8 g, 243.4 mmol, 1.2 eq) in t-BuOH (400 mL) at 28° C. The reaction was stirred at 28° C. for 1 hour. A solution of A-3 (40 g, 202.8 mmol, 1.0 eq) in t-BuOH (240 mL) at 28° C., then the mixture was heated to 75° C. and stirred for 6 hours. TLC showed the reaction was completed. The reaction was cooled to room temperature, filtered, and the filtrate was concentrated under vacuum to give crude product, which was purified by silica gel chromatography (petroleum ether/EtOAc=3/1˜1/1) to give A-4 (120.0 g).

### Step 4 (Synthesis of Compound A-5)

To a solution of A-4 (120.0 g, 448.7 mmol, 1.0 eq) in THE (2.4 L) was added sodium bicarbonate (120.1 g, 1.43 mol, 3.2 eq). Then to the reaction mixture was added CbzCl (153.1 g, 897.4 mmol, 2.0 eq) dropwise at 0° C. Then the reaction was stirred at room temperature for 4 hours. TLC showed the reaction was completed. The mixture was filtered and the solid was washed with DCM. The filtrate was concentrated to give A-5 (300.0 g, crude product), which was used in next step without further purification. LCMS: (ES, m/z): [M+H]⁺=356.1.

### Step 5 (Synthesis of Compound A-6)

To a solution of A-5 (300.0 g, 747.1 mmol, 1.0 eq) in acetone/H₂O (1:1, 600 mL) was added p-toluenesulfonic acid monohydrate (18.47 g, 97.13 mmol, 0.13 eq). The reaction was stirred at 65° C. for 2 hours. TLC showed the reaction was completed. Water was added and extracted with DCM (×3). The combined organic phase was washed with brine, dried over Na₂SO₄, filtered, and concentrated in vacuo to give a crude product, which was purified by silica gel column chromatography (petroleum ether/EtOAc=5:1) to give product A-6 (70.0 g). LCMS: (ES, m/z): [M+H]⁺=356.1.

### Step 6 (Synthesis of Compounds A & A′)

Under the atmosphere of nitrogen, to the solution of A-6 (22.0 g, 61.9 mmol, 1.0 eq) dissolved in THE (110 mL) was added dropwise lithium triethylhydridoborate (123.8 mL, 123.8 mmol, 2.0 eq, 1.0 M in THF) at −65° C. The reaction was stirred at −65° C. for 1 hour. HPLC showed the reaction was completed. The reaction mixture was quenched with saturated sodium bicarbonate (140 mL) at −40° C. to −30° C. Hydrogen peroxide (30% aqueous, 84.0 g) was added to the mixture dropwise at −10° C.-0° C. The mixture was stirred at 10° C. for 1 hour. Water was added and the mixture was extracted with EtOAc (×3). The combined organic phases were washed with brine, dried over Na₂SO₄, filtered, and concentrated in vacuo to give a residue, which was purified by silica gel column chromatography (petroleum ether/EtOAc=3:1), then further purified by trituration in DCM:EtOAc=1:1 to give A (9.0 g) and A′ (4.0 g).

The relevant characterization data for compound A was as follows: LCMS: (ES, m/z): [M+H]⁺=358.2. ¹HNMR (300 MHz, DMSO-d₆, ppm): δ 9.05 (brs, 1H), 7.38-7.36 (m, 5H), 5.92 (s, 1H), 5.12 (s, 2H), 4.57 (d, J=7.2 Hz, 1H), 4.15-4.13 (m, 1H), 2.91-2.88 (m, 1H), 2.28-2.14 (m, 1H), 1.88-1.80 (m, 1H), 1.76-1.69 (m, 2H), 1.52-1.43 (m, 11H).

The relevant characterization data for compound A′ was as follows: LCMS: (ES, m/z): [M+H]⁺=358.0. ¹HNMR (300 MHz, DMSO-d₆, ppm): δ 9.04 (brs, 1H), 7.37-7.33 (m, 5H), 5.88 (s, 1H), 5.10 (s, 2H), 4.43 (d, J=7.2 Hz, 1H), 4.21-4.11 (m, 1H), 3.20-3.02 (m, 1H), 2.03-1.48 (m, 5H), 1.52-1.40 (m, 10H).

### Step 7 (Synthesis of Compounds A1, A2)

A (22.5 g) was separated via chiral SFC [DAICELCHIRALPAK® AD with methanol (0.1% 7M ammonia in methanol)/Supercritical CO₂] to give A1 (8.4 g, P1) and A2 (7.3 g, P2).

The relevant characterization data for compound A1 was as follows: LCMS: (ES, m/z): [M+H]⁺=358.2. ¹HNMR (300 MHz, DMSO-d₆, ppm): δ 9.05 (brs, 1H), 7.38-7.36 (m, 5H), 5.93 (s, 1H), 5.12 (s, 2H), 4.57 (d, J=7.2 Hz, 1H), 4.15-4.13 (m, 1H), 2.91-2.88 (m, 1H), 2.28-2.14 (m, 1H), 1.88-1.80 (m, 1H), 1.76-1.69 (m, 2H), 1.52-1.43 (m, 1H). Optical rotation [α]_{D}+5.200 (c 1.0, MeOH). Chiral purity: 95.75% ee, retention time 2.824 min. Chiral SFC analysis was performed on a DAICEL CHIRALPAK® AD, 100*3.0 mm 3 m column heated to 35° C., eluted with a mobile phase of CO₂ and gradient of 5-40% MeOH (0.1% DEA), flowing at 1.5 mL/min.

The relevant characterization data for compound A2 was as follows: LCMS: (ES, m/z): [M+H]⁺=358.2. ¹HNMR (300 MHz, DMSO-d₆, ppm): δ 9.05 (brs, 1H), 7.45-7.30 (m, 5H), 5.92 (s, 1H), 5.12 (s, 2H), 4.57 (d, J=4.5 Hz, 1H), 4.19-4.10 (m, 1H), 2.95-2.83 (m, 1H), 2.23-2.14 (m, 1H), 1.88-1.43 (m, 4H), 1.42 (s, 9H). Chiral purity: 97.22% ee, retention time 3.026 min. Chiral SFC analysis was performed on a DAICEL CHIRALPAK® AD, 100*3.0 mm 3 μm column heated to 35° C., eluted with a mobile phase of CO₂ and gradient of 5-40% MeOH (0.1% DEA), flowing at 1.5 mL/min.

### Synthetic Route of Intermediate A as Follows (Route 2)

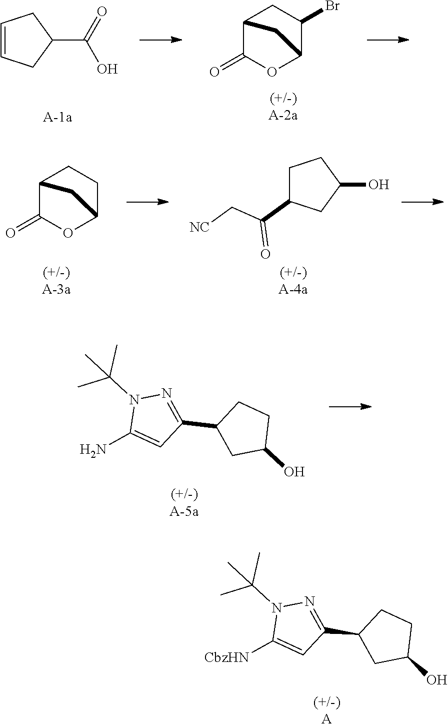

### Step 1 (Synthesis of Compound A-2a)

To a solution of DMSO (359.5 g, 4601.8 mmol) in chloroform (2 L) was added bromotrimethylsilane (710 g, 4637.5 mmol) at 0° C. under nitrogen atmosphere, and stirred for 3 hours at room temperature. Then a solution of cyclopent-3-ene-1-carboxylic acid (400 g, 3567.3 mmol) in chloroform (400 mL) was added and stirred for 10 minutes. N,N-diisopropylethylamine (594.7 g, 4601.8 mmol) was added at 10° C. The resulting mixture was heated to reflux and stirred for 20 hours. The mixture was diluted with dichloromethane, washed with water, hydrochloric acid solution (1N) and saturated brine, dried over sodium sulfate, filtered and concentrated to afford A-2a (413 g, crude).

### Step 2 (Synthesis of Compound A-3a)

To a solution of A-2a (139.4 g, 729.8 mmol) in toluene (1200 mL) were added 2,2-azobis(2-methylpropionitrile) (12 g, 73.0 mmol) and tributylstannane (240.9 g, 802.7 mmol), then stirred for 5 hours at 80° C. The reaction mixture was treated with saturated potassium fluoride aqueous and stirred at room temperature for 1 hour. The mixture was filtered and the filtrate was extracted with ethyl acetate. The organic layer was concentrated and purified by distillation under reduced pressure to give A-3a (125.76 g, crude).

### Step 3 (Synthesis of Compound A-4a)

Under nitrogen atmosphere, n-butyllithium (502.6 mL, 2.5M solution in hexane, 1256.5 mmol) was added into tetrahydrofuran (500 mL) at −65° C. Anhydrous acetonitrile (51.6 g, 1256.6 mmol) was added dropwise slowly to maintain the internal temperature below −55° C. The mixture was stirred for 1 hour at −65° C. A solution of A-3a (56.4 g, 502.6 mmol) in tetrahydrofuran (120 mL) was added dropwise slowly to maintain the internal temperature below −50° C. The mixture was stirred for 1 hour at −65° C. The mixture was quenched with water and diluted with hexane, then stirred for 10 minutes. The organic layer was discarded. The aqueous layer was adjusted to pH=3-4 with 2N hydrochloric acid solution and extracted with ethyl acetate/2-methyltetrahydrofuran (1/1, ×2). Organic layers were combined and dried over sodium sulfate, filtered and concentrated to give A-4a (206 g, crude).

### Step 4 (Synthesis of Compound A-5a)

To a solution of tert-butylhydrazine hydrochloride (99.2 g, 756.4 mmol) in tert-butanol (1000 mL) was added potassium tert-butoxide (106.1 g, 945.4 mmol), then stirred for 1 hour at room temperature. Then a solution of A-4a (96.55 g, 630.3 mmol) in tert-butanol (200 mL) was added. The resulting mixture was stirred at 85° C. for 24 hours. Two batches of such reaction were carried out. The mixture from two batches were cooled to room temperature, combined, diluted with ethyl acetate and stirred for 20 minutes. The mixture was filtered, and the filtrate was concentrated. The residue was diluted with tert-butyl methyl ether and treated with hydrochloric acid solution (3N). After stirring for 20 minutes, the organic layer was discarded and the aqueous layer was adjusted to pH=8-9 with 2N sodium hydroxide aqueous. The mixture was extracted with ethyl acetate (×3). Organic layers were combined, washed with brine, dried over sodium sulfate, filtered and concentrated to give A-5a (168 g, crude).

### Step 5 (Synthesis of Compound A)

To a solution of A-5a (160 g, 716.5 mmol) and sodium bicarbonate (300.9 g, 3582.3 mmol) in tetrahydrofuran (1500 mL) and acetonitrile (500 mL) was added benzyl chloroformate (135 g, 791.4 mmol) dropwise at 0° C., then stirred at room temperature for 20 hours. Filtered, the filtrate was concentrated. The residue was diluted with ethyl acetate, washed with water, brine, dried over sodium sulfate, filtered and concentrated. The crude was slurried with ethyl acetate/heptane ( 1/10) to give product A (80 g).

The relevant characterization data was as follows: LCMS (ESI, m/z): [M+H]⁺=358.2; ¹HNMR (400 MHz, DMSO-d₆, ppm): δ 9.03 (brs, 1H), 7.36-7.31 (m, 5H), 5.90 (s, 1H), 5.10 (s, 2H), 4.14-4.11 (m, 1H), 2.91-2.82 (m, 1H), 2.20-2.13 (m, 1H), 1.88-1.78 (m, 1H), 1.76-1.64 (m, 2H), 1.59-1.53 (m, 1H), 1.47-1.39 (m, 10H).

### Intermediates B1, B2

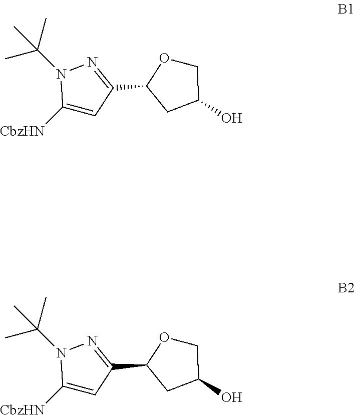 [SMILES: CC(C)(C)n1nc([C@H]2C[C@@H](O)CO2)cc1NC(=O)OCc1ccccc1 ; CC(C)(C)n1nc([C@@H]2C[C@H](O)CO2)cc1NC(=O)OCc1ccccc1]

### Synthetic Route of Intermediates B1, B2 as Follows

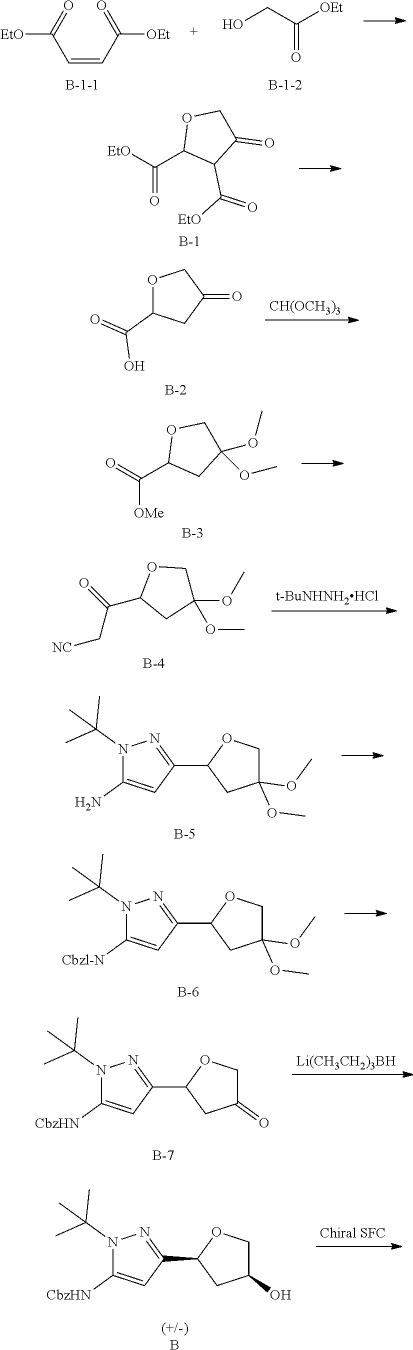

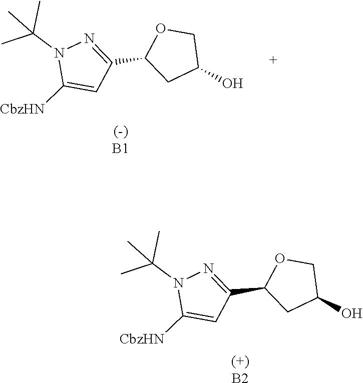 [SMILES: CC(C)(C)n1nc([C@H]2C[C@@H](O)CO2)cc1NC(=O)OCc1ccccc1 ; "(-)B1" ; CC(C)(C)n1nc([C@@H]2C[C@H](O)CO2)cc1NC(=O)OCc1ccccc1 ; "(+)B2"]

### Step 1 (Synthesis of Compound B-1)

To a suspension of sodium hydride (57.6 g, 1440.8 mmol, 60%) in tetrahydrofuran (1500 mL) was added B-1-2 (150 g, 1440.8 mmol) dropwise at ice bath. The reaction was stirred at room temperature for 1 hour. Then B-1-1 (235.68 g, 1368.7 mmol) was added dropwise. The reaction was stirred at 65° C. for 1 hour and then stirred at room temperature for 48 hours. After that, the reaction was diluted with ice-water and 1N hydrogen chloride. Most of tetrahydrofuran was remove and extracted with ethyl acetate (×3). The organic layer was concentrated and purified by silica gel column chromatography eluted with ethyl acetate in petroleum ether from 0% to 35%, kept at 35% to give compound B-1 (137 g, crude).

### Step 2 (Synthesis of Compound B-2)

To a flask containing B-1 (127 g, crude) was added 10% sulfuric acid (1270 mL). The mixture was stirred at 100° C. for 5 hours. Cooled to room temperature, the reaction was extracted with methanol in ethyl acetate (10%, ×5), dried over sodium sulphate and concentrated to give the crude compound B-2 (52.5 g).

### Step 3 (Synthesis of Compound B-3)

To a solution of B-2 (52.5 g, 403.5 mmol) in methanol (525 mL) were added trimethoxy methane (256.9 g, 2421.2 mmol) and p-methylbenzene-1-sulfonic acid hydrate (7.7 g, 40.4 mmol). And then the reaction was stirred at room temperature for 18 hours. The reaction was quenched with saturated sodium bicarbonate, then most of the methanol was removed, diluted with water and extracted with ethyl acetate (×3). The organic layer was collected, concentrated in vacuo, and dried to afford the title compound B-3 (51.8 g).

### Step 4 (Synthesis of Compound B-4)

To a solution of n-butyllithium (234.3 mL, 585.7 mmol) in tetrahydrofuran (550 mL) was added anhydrous acetonitrile (30.8 mL, 585.7 mmol) dropwise under nitrogen at −70° C. And the reaction was stirred at −70° C. for 2 hours. A solution of B-3 (55.7 g, 292.9 mmol) in tetrahydrofuran (100 mL) was added dropwise at −70° C. The reaction was stirred for an additional 1 hour at −70° C., cooled, quenched with water, neutralized with hydrochloride aqueous (2M) to pH=6-7, and extracted with ethyl acetate (×3). The combined organic layers were washed with saturated aq. sodium chloride (×2), dried over sodium sulphate, filtered and concentrated under vacuum to give the crude product B-4 (55.2 g).

### Step 5 (Synthesis of Compound B-5)

To a solution of tert-butyl hydrazine hydrochloride (41.43 g, 332.5 mmol) in ethanol (550 mL) was added potassium 2-methylpropan-2-olate (37.3 g, 332.5 mmol). And the reaction was stirred at room temperature for 1 hour. Then a solution of crude B-4 (55.2 g, 277.1 mmol) in ethanol (100 mL) was added at room temperature and the mixture was heated at 78° C. for 10 hours. The reaction was filtered and the filtrate was concentrated to give B-5 (77.8 g, crude), which was used without further purification. LCMS (ESI, m/z): [M+H]⁺=270.2

### Step 6 (Synthesis of Compound B-6)

To a solution of B-5 (77.8, crude) in acetonitrile (800 mL) was added benzyl chloroformate (98.55 g, 577.7 mmol) and the reaction was stirred at room temperature for 2 hours. Then sodium bicarbonate (77.7 g, 924.3 mmol) was added in portions and the reaction was stirred at room temperature for 48 hours. The resulted suspension was filtered and the filtrate was concentrated under vacuum to give the crude product B-6 (130 g, crude), which was used in next step without purification.

### Step 7 (Synthesis of Compound B-7)

To a solution of B-6 (130 g, crude) in acetone (750 mL) and water (750 mL) was added p-methylbenzene-1-sulfonic acid hydrate (61.3 g, 322.2 mmol) and the reaction was stirred at 60° C. for 4 hours. After that, water (500 mL) was added and extracted with dichloromethane. The organic layer was concentrated and purified by silica gel column chromatography eluting with ethyl acetate in petroleum ether from 0% to 35%, kept at 35% to give the title product B-7 (51 g). LCMS (ESI, m/z): [M+H]⁺=358.2

### Step 8 (Synthesis of Compound B)

To a solution of B-7 (51 g, 142.7 mmol) in tetrahydrofuran (500 mL) was added dropwise a solution of lithium triethylhydridoborate (11.7 mL, 11.7 mmol, 1.0 M in tetrahydrofuran) at a rate which maintained the internal temperature below −55° C. Then the reaction was stirred at −65° C. for 2.5 hours, and the reaction was warmed to 0° C. The reaction was quenched with water and extracted with ethyl acetate (×3). It was concentrated and purified by silica gel column chromatography (eluted with ethyl acetate in petroleum ether from 0% to 50%, kept at 50%) to give the title product B (45 g).

The relevant characterization data was as follows: LCMS (ESI, m/z): [M+H]⁺=360.2. ¹HNMR (400 MHz, CDCl₃, ppm): δ 7.38-7.35 (m, 5H), 6.42 (s, 1H), 6.27 (s, 1H), 5.20 (s, 2H), 5.08-5.06 (m, 1H), 4.47-4.45 (m, 1H), 4.00-3.93 (m, 2H), 2.45-2.38 (m, 1H), 2.32-2.29 (m, 1H), 1.58 (s, 9H).

### Step 9 (Synthesis of Compounds B1 & B2)

B (45 g) was separated via chiral SFC [Thar 200 preparative SFC(SFC-10), (S, S) Whelk O1, 300×50 mm I.D., 10 μm, with EtOH/Supercritical CO₂] to give B1 (16.61 g) and B2 (15.37 g).

The relevant characterization data for compound B1 was as follows: LCMS (ESI, m/z): [M+H]⁺=360.2. ¹HNMR (400 MHz, CDCl₃, ppm): δ 7.37 (m, 5H), 6.43 (s, 1H), 6.28 (s, 1H), 5.20 (s, 2H), 5.08-5.06 (m, 1H), 4.47-4.45 (m, 1H), 4.00-3.93 (m, 2H), 2.45-2.40 (m, 1H), 2.33-2.29 (m, 1H), 1.58 (s, 9H). Optical rotation [α]_{D}−20.594 (c 1.0, CHCl₃). Chiral purity: 99.10% ee, retention time 2.204 min. Chiral SFC analysis was performed on a Waters UPC2 analytical SFC (SFC-H), (S, S) Whelk O1, 300×50 mm ID, 10 μm column heated to 35° C., eluted with a mobile phase of CO₂ and gradient of 40% EtOH, flowing at 2.5 mL/min.

The relevant characterization data for compound B2 was as follows: LCMS (ESI, m/z): [M+H]⁺=360.2. ¹HNMR (400 MHz, CDCl₃, ppm): δ 7.38 (m, 5H), 6.37 (s, 1H), 6.30 (s, 1H), 5.20 (s, 2H), 5.09-5.07 (m, 1H), 4.48-4.45 (m, 1H), 4.00-3.93 (m, 2H), 2.46-2.39 (m, 1H), 2.33-2.29 (m, 1H), 1.58 (s, 9H). Optical rotation [α]_{D}+23.095 (c 1.0, CHCl₃). Chiral purity: 99.82% ee, retention time 2.581 min. Chiral SFC analysis was performed on a Waters UPC2 analytical SFC (SFC-H), (S, S) Whelk O1, 300×50 mm ID, 10 μm column heated to 35° C., eluted with a mobile phase of CO2 and gradient of 40% EtOH, flowing at 2.5 mL/min.

### Intermediate C

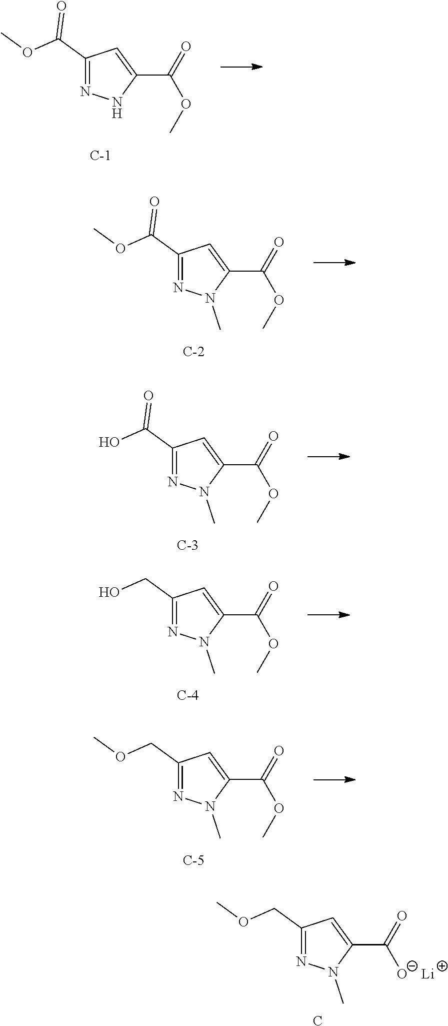

### Step 1 (Synthesis of Compound C-2)

To a solution of C-1 (150 g, 0.81 mol, 1 eq) in acetone (750 mL) at 0° C. were added K₂CO₃ (225 g, 1.63 mol, 2.0 eq), then Me₂SO₄ (118 g, 0.94 mol, 1.15 eq) and the mixture was allowed to warm to room temperature and stirred overnight. The mixture was filtered through a pad of Celite, washed with acetone, the filtrate was concentrated and re-dissolved in EtOAc, washed with water and brine, dried over Na₂SO₄, concentrated to give the residue, which was recrystallized in MTBE to give C-2 (145 g). LCMS (ESI, m/z): [M+H]⁺=199.1.

### Step 2 (Synthesis of Compound C-3)

To the solution of C-2 (25 g, 126 mmol, 1 eq) in 1,4-dioxane (200 mL) was added aqueous H₂SO₄ solution (450 mL, 1.5%). The mixture was heated to 80° C. for 18 hours. The mixture was cooled to room temperature, extracted with EtOAc (×2), then extracted with DCM (×2), the combined was dried over Na₂SO₄, concentrated to dryness, the residue was slurried in MTBE to give C-3 (10.8 g).

### Step 3 (Synthesis of Compound C-4)

Under the atmosphere of nitrogen, to a suspension of C-3 (25.0 g, 135.7 mmol, 1 eq) in THE (200 mL) was added borane tetrahydrofuran complex solution (407 mL, 407 mmol 3.0 eq) dropwise at 0° C., and the mixture was heated to 40° C. and stirred for 5 hours. After being cooled to 0° C., methanol was added dropwise and the mixture was warmed to room temperature and stirred overnight. The mixture was concentrated and the residue was purified by silica gel (PE:EtOAc 5:1 to 2:1) to give C-4 (20.0 g). LCMS (ESI, m/z): [M+H]⁺=171.1.

### Step 4 (Synthesis of Compound C-5)

To the solution of C-4 (15.0 g, 88.1 mmol, 1 eq) in DMF (150 mL) were added Ag₂O (51.1 g, 220.4 mmol, 2.5 eq) and iodomethane (25.0 g, 176.3 mmol, 2 eq), the mixture was stirred at room temperature overnight. The mixture was diluted with EtOAc (500 mL), filtered through a pad of Celite, the filtrate was washed with water and brine, dried over Na₂SO₄, concentrated to dryness, the residue was purified by silica gel (PE:EtOAc 20:1 to 3:1) to give C-5 (14.3 g). LCMS (ESI, m/z): [M+H]⁺=185.1.

### Step 5 (Synthesis of Compound C)

To a solution of C-5 (3.0 g, 16.3 mmol, 1.0 eq) in THE (30 mL) was added LiOH (410 mg, 17.1 mmol, 1.05 eq) in water (10 mL) at room temperature overnight. The solvent was removed under reduced pressure and the residue was dispersed in THF, concentrated, the crude was slurried with MTBE to give C (2.5 g).

The relevant characterization data was as follows: LCMS (ESI, m/z): [M−Li]⁻=169.0. ¹HNMR (400 MHz, DMSO-d₆, ppm) δ 6.28 (s, 1H), 4.23 (s, 2H), 4.00 (s, 3H), 3.20 (s, 3H).

### Intermediate C1

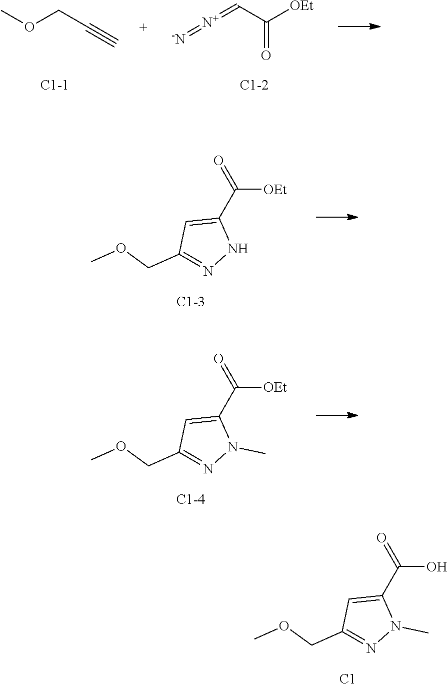

### Step 1 (Synthesis of Compound C1-3)

To a solution of 3-methoxyprop-1-yne (18.7 g, 266.8 mmol) in toluene (190 mL) was added ethyl 2-diazoacetate (33.49 g, 293.5 mmol) at 20˜25° C. dropwise. Then the resulted mixture was heated to 110° C. and stirred for 6 hours. After being cooled to room temperature, the solvent was removed in vacuo. The residue was purified by silica gel chromatography (PE:EtOAc=1:1) to afford C1-3 (17.8, crude). LCMS (ESI, m/z): [M+H]⁺=184.8.

### Step 2 (Synthesis of Compound C1-4)

In a 500 mL three-neck flask, C1-3 (6.0 g, 32.6 mmol), acetone (60 mL) and potassium carbonate (13.5 g, 97.7 mmol) were added, then followed by iodomethane (9.25 g, 65.2 mmol) dropwise. The reaction mixture was stirred at 25° C. for 3.5 hours with rubber stopper in a closed environment. The reaction mixture was filtered and the filtrate was concentrated in vacuo. The residue was purified by silica gel chromatography (PE EtOAc 3:1) to afford C1-4 (1.8 g). LCMS (ESI, m/z): [M+H]⁺=199.2.

### Step 3 (Synthesis of Compound C1)

To a stirred solution of C1-4 (1.6 g, 8.1 mmol) in tetrahydrofuran (8 mL) and water (8 mL) was added lithium hydroxide hydrate (406.4 mg, 9.7 mmol). The mixture was stirred at 25° C. for 2.5 hours. The reaction mixture was adjusted pH=1 with 2 N hydrochloric acid and extracted with ethyl acetate (×2). The combined organic layers were washed with brine, dried over anhydrous sodium sulphate, filtered and concentrated in vacuo to afford C1 (1.2 g).

The relevant characterization data was as follows: LCMS (ESI, m/z): [M+H]⁺=171.0. ¹HNMR (400 MHz, DMSO-d₆, ppm): δ 13.32 (brs, 1H), 6.75 (s, 1H), 4.32 (s, 2H), 4.04 (s, 3H), 3.24 (s, 3H).

### Intermediate D

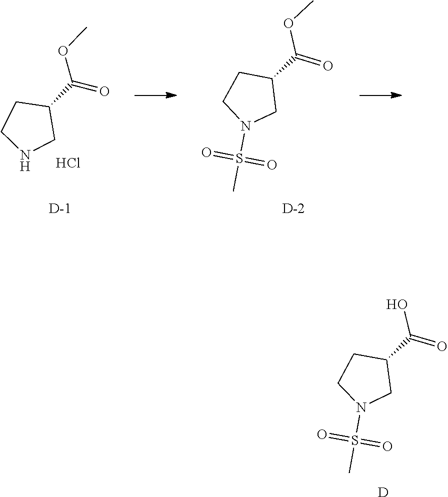

### Step 1 (Synthesis of Compound D-2)

To a solution of D-1 (0.9 g, 5.4 mmol, 1.0 eq) in DCM (20 mL) were added Et₃N (1.9 g, 16.3 mmol, 3.0 eq) and MsCl (1.1 g, 10.8 mmol, 2.0 eq) at 0° C. The reaction mixture was stirred at room temperature for 15 hours. TLC showed the reaction was completed. Then the mixture was quenched with water. The aqueous phase was extracted with DCM. The combined organic phase was washed with brine, dried over Na₂SO₄, filtered and concentrated to give a residue which was purified by silica gel chromatography (PE:EtOAc=1:1) to give D-2 (880 mg).

### Step 2 (Synthesis of Compound D)

To a solution of D-2 (800 mg, 3.8 mmol, 1.0 eq) in THE (5 mL) were added LiOH·H₂O (243 mg, 5.8 mmol, 1.5 eq) and H₂O (5 mL). The reaction mixture was stirred at room temperature for 1 hour. HPLC showed the reaction was completed. Then the mixture was concentrated. The residue added water. The mixture was extracted with EtOAc. The aqueous phase was acidified to pH 3 with aqueous potassium bisulfate solution. The aqueous phase was extracted with DCM (×6). The combined organic phase was dried over anhydrous Na₂SO₄, filtered and concentrated to give compound D (600 mg).

The relevant characterization data was as follows: ¹HNMR (400 MHz, CDCl₃, ppm): δ 3.63-3.61 (m, 2H), 3.50-3.37 (m, 2H), 3.24-3.17 (m, 1H), 2.87 (s, 3H), 2.31-2.26 (m, 2H).

The intermediates D1-8 below were synthesized followed the similar route of intermediate D.

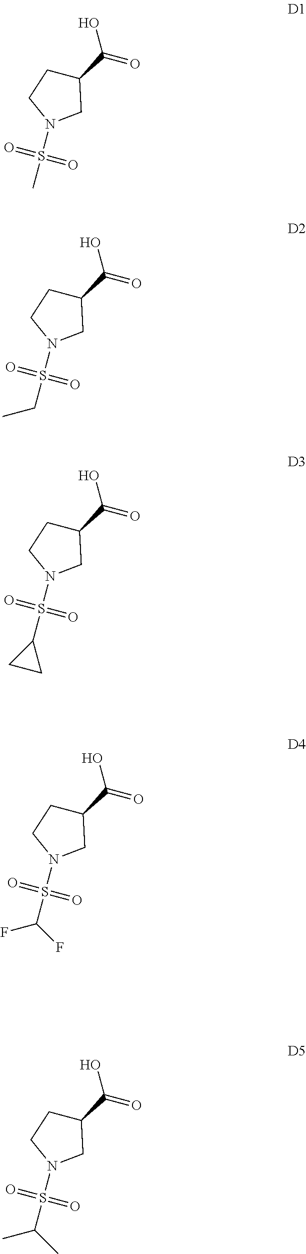

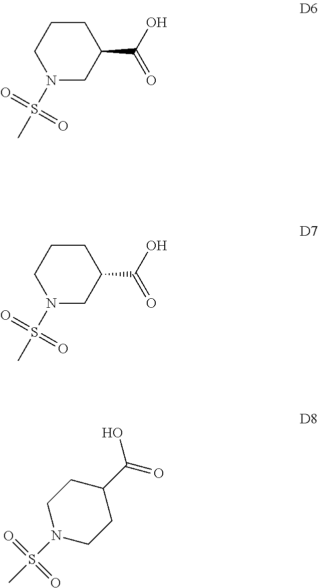

### Intermediate E

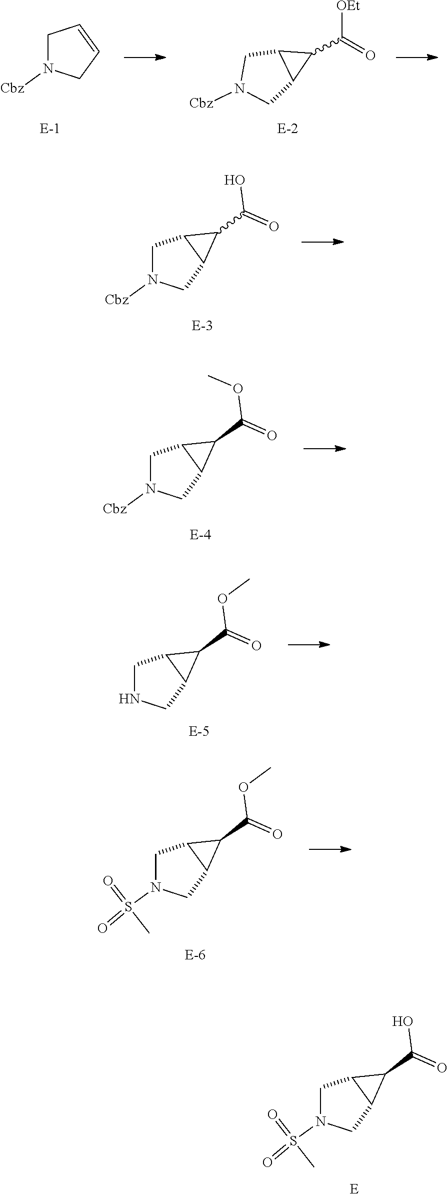

### Step 1 (Synthesis of Compound E-2)

Under nitrogen atmosphere, to a solution of E-1 (10.0 g, 49.2 mmol, 1.0 eq) in 1,2-dichloroethane (100 mL) was added rhodium (II) acetate dimer (120 mg, 0.27 mmol, 0.0054 eq) at room temperature, then the mixture was heated 80° C. and a solution of ethyl diazoacetate (29.0 g, 224.3 mmol, 4.56 eq) in 1,2-dichloroethane (300 mL) was added dropwise. The mixture was stirred for 2 hours. After completion of the reaction, the mixture was concentrated to afford E-2 (30 g, crude), which was used in next step without further purification.

### Step 2 (Synthesis of Compound E-3)

To a solution of E-2 (30.0 g, crude) in methanol (250 mL) and water (75 mL) was added LiOH (5.3 g, 0.22 mol) at room temperature. Then the mixture was stirred for 2 hours at room temperature. The mixture was adjusted to pH 6 with 2 N HCl and extracted with DCM:MeOH (10:1, ×3), the combined organic layers were washed with brine, dried over Na₂SO₄ and concentrated. The residue was purified by column chromatography on silica gel (eluted with PE:EtOAc=3:1) to give E-3 (10.0 g).

### Step 3 (Synthesis of Compound E-4)

To a solution of E-3 (10.0 g, 38.3 mmol, 1.0 eq) in DMF (100 mL) at room temperature was added K₂CO₃ (10.6 g, 76.6 mmol, 2.0 eq) and MeI (6.5 g, 46.0 mmol, 1.2 eq). Then the mixture was stirred for 2 hours at room temperature. The reaction mixture was poured into water and the mixture was extracted with EtOAc (×3), the combined organic layers were washed with brine, dried over Na₂SO₄ and concentrated, the residue was purified by column chromatography on silica gel (PE:EtOAc=25:1) to afford E-4 (5.0 g).

### Step 4 (Synthesis of Compound E-5)

To a solution of E-4 (1.5 g, 5.4 mmol, 1.0 eq) in MeOH (20 mL) was added Pd/C (750 mg, 10% wt), the mixture stirred for 2 hours at room temperature. The mixture was filtered through a pad of Celite, the filtrate was concentrated to give E-5 (590 mg), which was used in next step without further purification. LCMS (ESI, m/z): [M+H]⁺=142.2.

### Step 5 (Synthesis of Compound E-6)

To a solution of E-5 (590 mg, 4.18 mmol, 1.0 eq) and TEA (846 mg, 8.36 mmol, 2.0 eq) in DCM (6 mL) was added MsCl (575 mg, 5.02 mmol, 1.2 eq) dropwise at 0° C., then the mixture was stirred for 2 hours at room temperature. The reaction mixture was poured into water and the mixture was extracted with EtOAc (×3), the combined organic layers were washed with brine, dried over Na₂SO₄ and concentrated. The residue was triturated with PE/EtOAc (3:1, 2 mL) for 30 minutes to afford E-6 (650 mg).

The relevant characterization data was as follows: LCMS (ESI, m/z): [M+H]⁺=220.0. ¹HNMR (300 MHz, CDCl₃, ppm): δ 3.68 (s, 3H), 3.63 (d, J=9.9 Hz, 2H), 3.41 (d, J=9.6 Hz, 2H), 2.83 (s, 3H), 2.16-2.12 (m, 2H), 1.78 (t, J=3.0 Hz, 1H).

### Step 6 (Synthesis of Compound E)

To a solution of E-6 (200 mg, 0.91 mmol, 1.0 eq) in MeOH (2 mL) and water (0.6 mL) was added LiOH (27 mg, 1.1 mol, 1.2 eq). Then the mixture stirred at room temperature for 2 hours. The mixture was adjusted to pH 2˜3 with 2 M HCl in EtOAc and concentrated to give E-7 (300 mg, crude), which was used directly in next step. LCMS (ESI, m/z): [M+H]⁺=206.0.

### Intermediate F

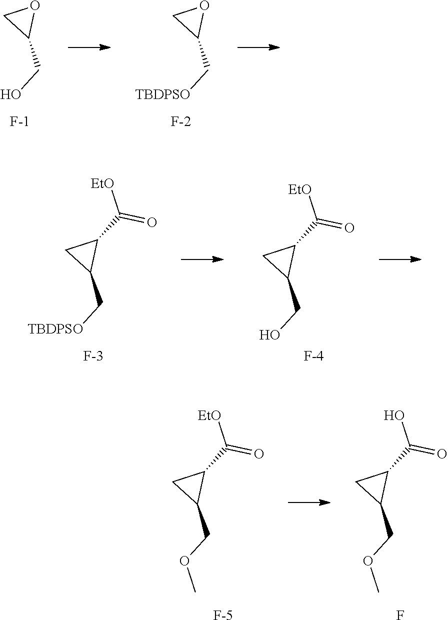

### Step 1 (Synthesis of Compound F-2)

To a solution of F-1 (10.0 g, 135 mmol, 1 eq) in DCM (50 mL) was added imidazole (16.5 g, 243 mmol, 1.8 eq) at 0° C., then TBDPSCl (48.2 g, 175 mmol, 1.3 eq) in DCM (50 mL) dropwise and the mixture was allowed to warm to room temperature and stirred for 2 hours. The reaction was quenched with saturated aqueous NH₄Cl solution, extracted with DCM. The organic phase washed water and NH₄Cl solution, dried over Na₂SO₄, concentrated to dryness, the residue was purified on silica to give F-2 (33.0 g).

### Step 2 (Synthesis of Compound F-3)

Under the atmosphere of nitrogen, to the solution of F-2 (20 g, 64 mmol, 1 eq) in toluene (200 mL) was added triethyl phosphonoacetate (28.7 g, 128 mmol) at 0° C., then NaH (5.3 g, 131 mmol, 2.05 eq) was added in portions. After addition, the reaction was heated to 80° C. within 1 hour and stirred overnight. The mixture was quenched with saturated aqueous NH₄Cl solution, extracted with EtOAc (×2), the organic phase was washed with water and brine, dried over Na₂SO₄, concentrated, the residue was purified on silica get to F-3 (15.0 g).

### Step 3 (Synthesis of Compound F-4)

To a solution of F-3 (15.0 g, 39.2 mmol, 1 eq) in THE (80 mL) was added the solution of TBAF in THE (47 mL, 47 mmol, 1 mol/L, 1.2 eq) dropwise at 0° C., and the mixture was allowed to warm to room temperature and stirred for 3 hours. The mixture was concentrated and the residue was purified on silica get to F-4 (5.6 g).

### Step 4 (Synthesis of Compound F-5)

To the solution of F-4 (3.0 g, 20.8 mmol, 1 eq) in DMF (30 mL) was added Ag₂O (14.5 g, 62.4 mmol) and MeI (7.4 g, 52.0 mmol) and the mixture was stirred at room temperature overnight. The mixture was diluted with EtOAc and filtered through a pad of Celite, the filtrate was washed with water and brine dried over Na₂SO₄, concentrated to dryness, the residue was purified on silica gel to give F-5 (3.0 g).

### Step 5 (Synthesis of Compound F)

To a solution of F-5 (3.0 g, 19.0 mmol, 1.0 eq) in THE (200 mL) was added LiOH (908 mg, 37.9 mmol, 2 eq) in water (10 mL) at room temperature overnight. THE and water was removed under reduced pressure, the residue was dispersed in THF, a solution of HCl in EtOAc (3M) was added and stirred at room temperature for 30 minutes. The mixture was concentrated and the crude was purified by silica gel column chromatography to give F (2.1 g).

The relevant characterization data was as follows: ¹HNMR (400 MHz, CDCl₃, ppm): δ 3.40-3.36 (m, 1H), 3.35 (s, 3H), 3.29-3.25 (m, 1H), 1.79-1.74 (m, 1H), 1.59-1.55 (m, 1H), 1.29-1.24 (m, 1H), 0.96-0.91 (m, 1H).

The synthesis of intermediate F1 was followed the similar synthetic method of intermediate F.

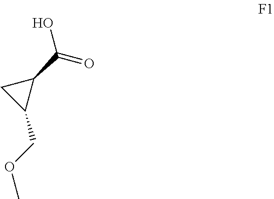

### Intermediate G

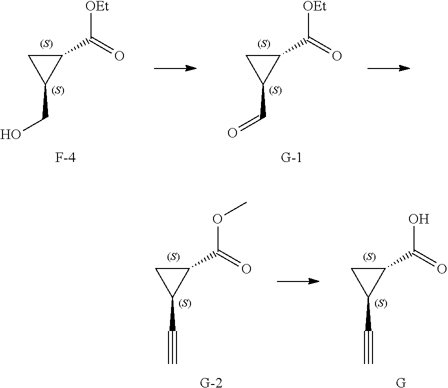

### Step 1 (Synthesis of Compound G-1)

To a solution of F-4 (3.0 g, 20.8 mmol, 1.0 eq) in CHCl₃ (60 mL) was added Dess-Martin periodinane (13.2 g, 31.2 mmol, 1.4 eq) in portions at 0° C. Then the mixture stirred for 4 hours at room temperature. The mixture was filtered through a Celite pad, and the filtrate was diluted with the addition of the saturated aqueous Na₂S₂O₃ and NaHCO₃ solution (1:1), the mixture was extracted with EtOAc, the combined organic layers were washed with brine, dried over Na₂SO₄ and concentrated. The residue was purified by column chromatography on silica gel (PE:EtOAc=30:1) to give G-1 (1.6 g).

### Step 2 (Synthesis of Compound G-2)

Under N2, to a solution of G-1 (1 g, 7.0 mmol, 1.0 eq) in MeOH (5 mL) was added dimethyl (1-diazo-2-oxopropyl)phosphonate (1.6 g, 8.4 mmol, 1.2 eq) and K₂CO₃ (1.9 g, 14.0 mmol, 2 eq). The reaction mixture was stirred at room temperature for 1 hour. TLC showed the reaction was completed. Then to the mixture was added with water, extracted with MTBE (×3). The combined organic phase was washed with water and brine, dried over Na₂SO₄, filtered and concentrated to give a residue which was purified by silica gel chromatography (hexane:MTBE=5:1) to give G-2 (500 mg).

The relevant characterization data was as follows: ¹HNMR (400 MHz, CDCl₃, ppm): δ 3.70 (s, 3H), 1.98-1.90 (m, 1H), 1.88-1.83 (m, 2H), 1.40-1.35 (m, 1H), 1.29-1.22 (m, 1H).

### Step 3 (Synthesis of Compound G)

To a solution of G-2 (200 mg, 1.6 mmol, 1.0 eq) in MeOH (2 mL) and water (1 mL) was added NaOH (96 mg, 2.4 mmol, 1.5 eq). The reaction mixture was stirred at room temperature for 1 hour. TLC showed the reaction was completed. The mixture was cooled to 0° C. and acidified to pH 2-3 with concentrated hydrochloric acid, concentrated, and washed with THF. The THF phase was dried over Na₂SO₄, filtered and concentrated to give G (180 mg, crude), which was used directly in next step.

### Intermediate H

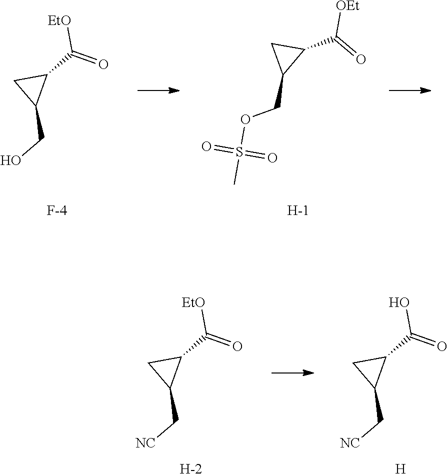

### Step 1 (Synthesis of Compound H-1)

To a solution of F-4 (2.0 g, 13.9 mmol, 1 eq) in CHCl₃ (20 mL) and Et₃N (2.8 g, 27.7 mmol, 2.0 eq) was added MsCl (2.4 g, 20.8 mmol, 1.5 eq) dropwise at 0° C. The resulted mixture was stirred at 0° C. for 2 hours. TLC showed the completion of the reaction. The mixture was quenched with aqueous saturated NH₄Cl solution, extracted with DCM (20 mL×2), the combined organic phase was washed with water (20 mL×2) and brine, dried over Na₂SO₄ and concentrated, the residue was purified on silica (PE:EtOAc=10:1 to 3:1) to give H-1 (2.0 g).

### Step 2 (Synthesis of Compound H-2)

To a solution of H-1 (400 mg, 1.8 mmol, 1.0 eq) in acetonitrile (4 mL) was added TBAF (5.4 mL, 1 M in THF, 5.4 mmol) and trimethylsilanecarbonitrile (893 mg, 9.0 mmol), the resulted solution was heated at 40° C. for 16 hours. TLC showed the completion of the reaction. The mixture was cooled to room temperature and quenched with water, extracted with EtOAc (×3), the combined organic phase was washed with water and brine, dried over Na₂SO₄ and concentrated, the residue was purified by silica gel column chromatography (PE:EtOAc=20:1 to 5:1) to give H-2 (200 mg).

The relevant characterization data was as follows: ¹HNMR (400 MHz, CDCl₃, ppm): δ 4.14 (q, J=7.2 Hz, 2H), 2.54 (d, J=5.6 Hz, 2H), 1.74-1.62 (m, 2H), 1.34-1.30 (m, 1H), 1.27 (t, J=7.2 Hz, 3H), 0.99-0.94 (m, 1H).

### Step 3 (Synthesis of Compound H)

To a solution of H-2 (200 mg, 1.3 mmol, 1.0 eq) in MeOH (4 mL) was added LiOH·H₂O (110 mg, 2.6 mmol, 2.0 eq) in water (2 mL) and stirred at room temperature for 3 hours. The solvent was removed under reduced pressure, the residue was added EtOAc. The pH was adjusted to 1 by aqueous HCl solution (6 M), extracted with EtOAc (×3), the combined organic phase was dried over Na₂SO₄ and concentrated to give H (120 mg, crude), which was used in next step.

### Intermediate I

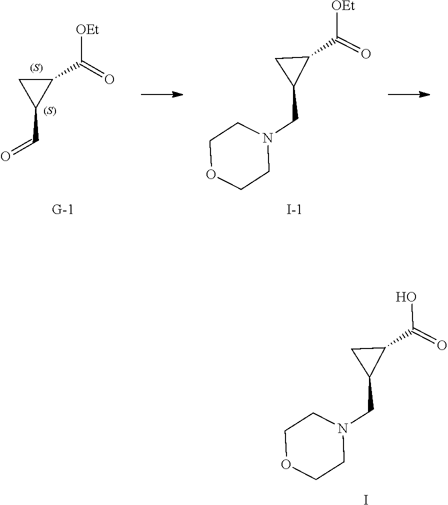

### Step 1 (Synthesis of Compound I-1)

Under the atmosphere of nitrogen, to the solution of F-4 (500 mg, 3.5 mmol, 1 eq) and morpholine (398 mg, 4.6 mmol, 1.3 eq) in DCM (5 mL), NaBH(OAc)₃ (969 g, 4.6 mmol, 1.3 eq) was added in potions at 0° C., then heated at room temperature for 6 hours. To the mixture was added aqueous saturated NaHCO₃ solution, extracted with DCM (×2), the combined organic phase was washed with water and brine, dried over Na₂SO₄, concentrated and purified on silica (PE:EtOAc=10:1 to EtOAc) to give I-1 (470 mg).

¹HNMR (400 MHz, CDCl₃, ppm): δ 4.15-4.08 (m, 2H), 3.74-3.40 (m, 4H), 2.54-2.47 (m, 4H), 2.40-2.25 (m, 2H), 1.57-1.50 (m, 1H), 1.45-1.40 (m, 1H), 1.29-1.21 (m, 4H), 0.79-0.72 (m, 1H).

### Step 2 (Synthesis of Compound I)

To a solution of I-1 (470 mg, 2.2 mmol, 1 eq) in MeOH (5 mL) was added the solution of NaOH (97 mg, 2.4 mmol, 1.1 eq) in water (2.5 mL), the mixture was stirred at room temperature for 3 hours. TLC showed the completion of the reaction. The solvent was removed under reduced pressure. The residue was added THF, the solution of HCl in EtOAc (3M) was added and stirred for 30 minutes. Concentrated to give crude I (500 mg), which was used for next step without any further purification.

### Intermediate J

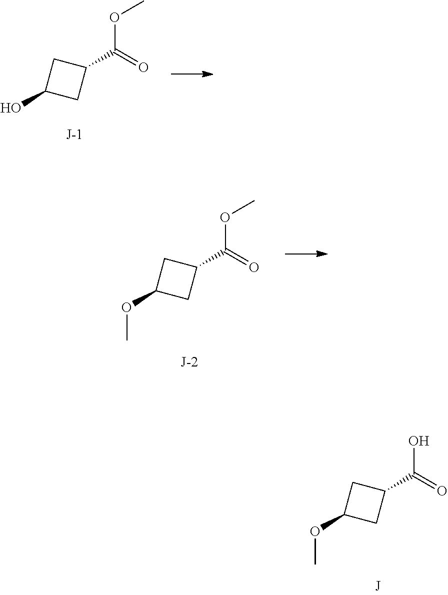

### Step 1 (Synthesis of Compound J-2)

To the solution of J-1 (1.0 g, 7.68 mmol) in DMF (40 mL) was added AgO (3.8 g, 30.7 mmol) and CH₃I (4.4 g, 30.7 mmol). The reaction was stirred at 45° C. overnight. The reaction was washed with water and extracted with ethyl acetate (80 mL). The organic layer was dried over sodium sulfate and concentrated, then purified by silica gel column chromatography eluted with PE/EA=1:1 to give J-2 (1.0 g).

The relevant characterization data was as follows: ¹HNMR (400 MHz, CDCl₃, ppm): δ 4.06-4.00 (m, 1H), 3.62 (s, 3H), 3.17 (s, 3H), 3.00-2.94 (m, 1H), 2.47-2.40 (m, 2H), 2.19-2.11 (m, 2H).

### Step 2 (Synthesis of Compound J)

To a solution of J-2 (500 mg, 3.5 mmol) in THF (5 mL) were added H₂O (5 mL) and LiOH·H₂O (145.5 mg, 3.5 mmol), the reaction was stirred at room temperature for 3 hours. The residue was diluted in EtOAc and added 1N HCl solution until pH=3, the organic layer was washed with water. The organic layer was concentrated in vacuo and dried to give J (400 mg), which was used directly.

### Intermediate K

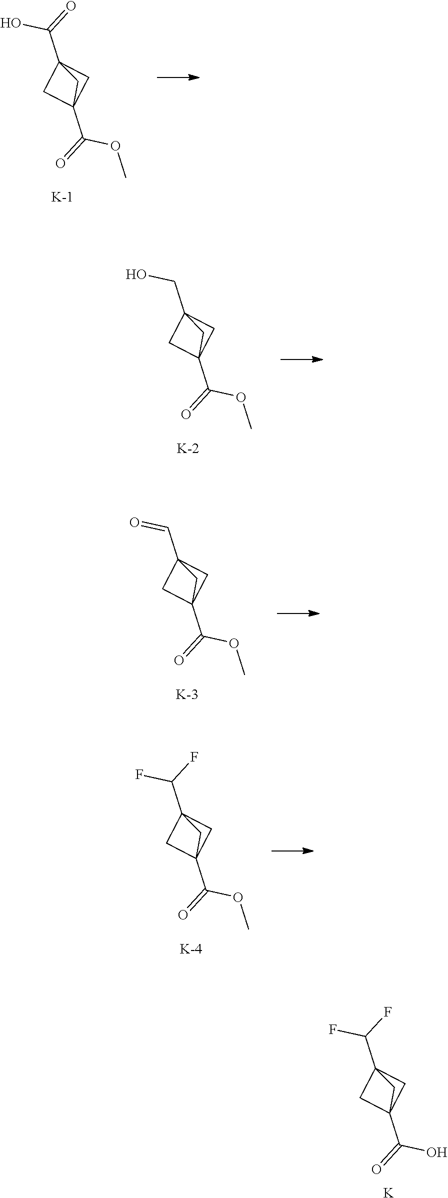

### Step 1 (Synthesis of Compound K-2)

Under N2, to a solution of K-1 (15.0 g, 88.2 mmol, 1.0 eq) in THE (300 mL) was added the solution of BH₃-THF in THE (1M, 105.8 mL, 105.8 mmol, 1.2 eq) dropwise at 0° C. The reaction was stirred at room temperature overnight. TLC showed the reaction was completed. The reaction was cooled to 0° C., quenched with MeOH and stirred at room temperature for 30 min. The mixture was concentrated and purified by column chromatography (PE:EtOAc=5:1) to give K-2 (10.1 g).

### Step 2 (Synthesis of Compound K-3)

To the solution of K-2 (10.0 g, 64.0 mmol, 1.0 eq) in DCM (200 mL) was added Dess-Martin periodinane (54.3 g, 128.1 mmol, 2.0 eq) in portions at 0° C. The reaction was stirred at room temperature for 2 hours. TLC showed the reaction was completed. The mixture was filtered through a pad of Celite, washed with DCM (×3), the filtrate was washed with aqueous 10% NaHCO₃ solution and then brine, dried over Na₂SO₄, filtered and concentrated in vacuo, the residue was purified by column chromatography (PE:EtOAc=7:1) to give K-3 (3.6 g).

### Step 3 (Synthesis of Compound K-4)

Under N2, to a solution of K-3 (2.5 g, 16.2 mmol, 1.0 eq) in DCM (40 mL) was added DAST (7.8 g, 48.6 mmol, 3.0 eq) dropwise at −78° C. The reaction was raised to room temperature and stirred for 2 hours. TLC showed the reaction was completed. The mixture was cooled to 0° C., quenched with aqueous 10% NaHCO₃ solution, extracted with DCM (×3), the organic phase washed with water and then brine, dried over Na₂SO₄, filtered and concentrated in vacuo to give K-4 (2.6 g), which was used in next step without further purification.

### Step 4 (Synthesis of Compound K)

To a solution of K-4 (2.6 g, 14.8 mmol, 1.0 eq) in MeOH (50 mL) and H₂O (10 mL) was added LiOH·H₂O (1.2 g, 29.5 mmol, 2.0 eq). The reaction was stirred at room temperature for 3 hours. LCMS showed the reaction was completed. The mixture was concentrated and diluted with water, extracted with MTBE (×3). The aqueous phase was adjusted pH to 1 with 1N HCl, extracted with EtOAc (×3), washed with water and then brine, dried over Na₂SO₄, filtered and concentrated in vacuo to give K (2.0 g).

The relevant characterization data was as follows: ¹HNMR (400 MHz, CDCl₃, ppm): δ 5.72 (t, J=56.0 Hz, 1H), 2.18 (s, 6H).

### Intermediate L

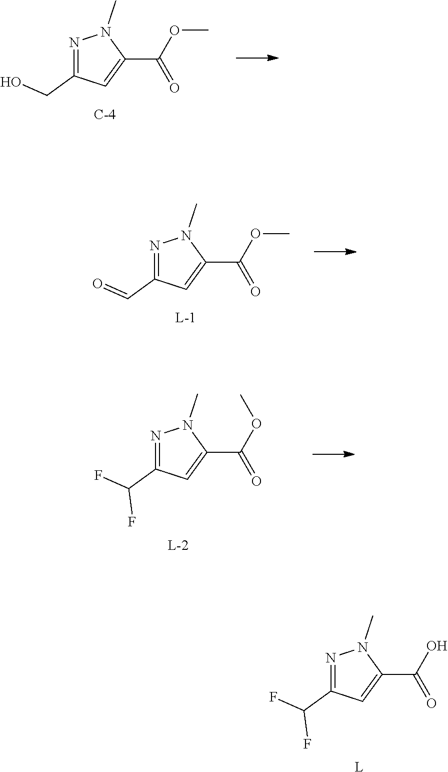

### Step 1 (Synthesis of Compound L-1)

To a solution of C-4 (500 mg, 2.9 mmol) in DCM (50 mL) was added manganese dioxide (2554.5 mg, 29.4 mmol). The reaction was stirred at room temperature for 16 hours. The reaction mixture was filtered. The filtrate was concentrated to obtain the product L-1 (480 mg). LCMS (ESI, m/z): [M+H]⁺=169.2

### Step 2 (Synthesis of Compound L-2)

To a solution of L-1 (0.50 g, 3.0 mmol) in DCM (20 mL) were added bis(2-methoxyethyl)aminosulfur trifluoride (2.0 g, 8.9 mmol) at 0° C. and the reaction was stirred at room temperature overnight. The reaction mixture was quenched by the addition of the saturated aqueous NaHCO₃. The organic layer was separated, washed with further saturated NaCl solution, and concentrated in vacuo. The residue was chromatographed on silica gel (PE/EA 2:1) to give L-2 (500 mg).

The relevant characterization data was as follows: ¹HNMR (400 MHz, CDCl₃, ppm): δ 7.03 (s, 1H), 6.81-6.58 (t, J=56.0 Hz, 1H), 4.20 (s, 3H), 3.91 (s, 3H).

### Step 3 (Synthesis of Compound L)

To a solution of L-2 (500 mg, 2.6 mmol) in THE (5 mL) was added H₂O (5 mL) and LiOH·H₂O (110.5 mg, 2.6 mmol), the reaction was stirred at room temperature for 3 hours. The reaction mixture was dissolved in EtOAc and added 1N HCl solution to adjust pH=3, further washed with water. The organic layer was collected, concentrated in vacuo and dried to afford L (400 mg). LCMS (ESI, m/z): [M+H]⁺=199.2

### Intermediate M

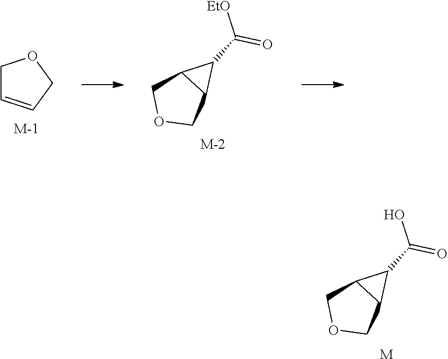

### Step 1 (Synthesis of Compound M-2)

To the solution of M-1 (2 g, 28.5 mmol) in DCM (40 mL) were added rhodium acetate dimer (0.6 g, 1.4 mmol) and then ethyl 2-diazoacetate (3.26 g, 28.5 mmol) dissolved in DCM (20 mL) was added slowly. The reaction was stirred at room temperature overnight. The mixture was concentrated in vacuo and the residue was purified by silica gel column chromatography eluting with PE/EA=1:1 to offer M-2 (600.00 mg).

The relevant characterization data was as follows: ¹HNMR (400 MHz, CDCl₃, ppm): δ 4.16-4.11 (q, J=7.2 Hz, 2H), 3.94-3.92 (d, J=8.8 Hz, 2H), 3.76-3.74 (d, J=8.8 Hz, 2H), 2.17-2.15 (m, 2H), 1.61-1.60 (t, J=3.2 Hz, 1H), 1.29-1.25 (t, J=7.2 Hz, 3H).

### Step 2 (Synthesis of Compound M)

In a 50 mL round-bottomed flask was added M-2 (100 mg, 0.64 mmol) and LiOH (26.9 mg, 0.64 mmol) in THE (5 mL), H₂O (5 mL). The reaction mixture stirred at room temperature for 2 hours. The mixture was adjusted to pH=3 with 1N HCl aq. The aqueous layer was extracted with ethyl acetate (×3). The organic layer was dried by Na₂SO₄, filtered and concentrated to give M (70 mg, crude), which was used directly in next step. LCMS (ESI, m/z): [M+H]⁺=129.2

### Intermediate N

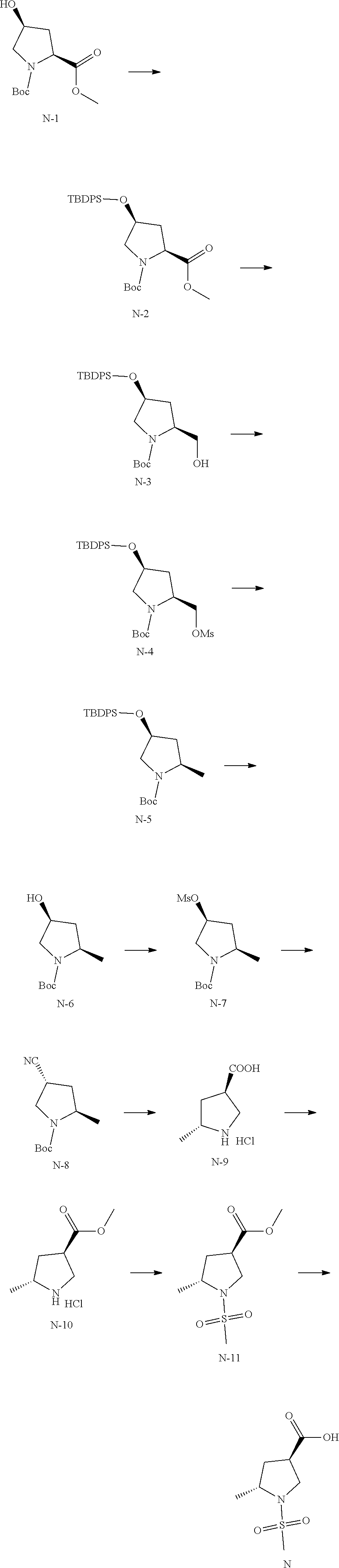

### Step 1 (Synthesis of Compound N-2)

To the solution of N-1 (20.0 g, 81.5 mmol, 1.0 eq) in DMF (140 mL) were added imidazole (6.7 g, 97.8 mmol, 1.2 eq) and TBDPSCl (31.4 g, 114.2 mmol, 1.4 eq) at 0° C. under N2 atmosphere. The reaction mixture was stirred at room temperature for 15 hours. TLC showed the reaction was completed. Then the mixture was poured into H₂O and extracted with EtOAc (×3). The combined organic layers were washed with brine, dried over Na₂SO₄, filtered and concentrated to give a residue which was purified by silica gel column chromatography (PE:EtOAc=5:1) to give compound N-2 (20.0 g). LCMS (ESI, m/z): [M+Na]⁺=506.1.

### Step 2 (Synthesis of Compound N-3)

To a solution of N-2 (20.0 g, 41.3 mmol, 1.0 eq) in THE (60 mL) and EtOH (60 mL) was added NaBH₄ (3.1 g, 82.7 mmol, 2.0 eq) and CaCl₂) (4.6 g, 41.3 mmol, 1.0 eq) at 0° C. The reaction mixture was stirred at room temperature for 3 hours. HPLC showed the reaction was completed. Then the mixture was treated with 1N HCl (60 mL) at 0° C. and extracted with EtOAc (×3). The combined organic layers were washed with brine, dried over Na₂SO₄, filtered and concentrated to give N-3 (18.0 g).

### Step 3 (Synthesis of Compound N-4)

To a solution of N-3 (13.0 g, 28.5 mmol, 1.0 eq) in DCM (156 mL) was added TEA (8.7 g, 85.6 mmol, 3.0 eq) and MsCl (4.9 g, 42.8 mmol, 1.5 eq) at 0° C. under N2 atmosphere. The mixture was stirred at room temperature for 15 hours. HPLC showed the reaction was completed. Then the mixture was washed with water and then brine, dried over Na₂SO₄, filtered and concentrated to give N-4 (14.0 g).

### Step 4 (Synthesis of Compound N-5)

To a solution of N-4 (14.0 g, 26.2 mmol, 1.0 eq) in THE (56 mL) was added LiBHEt₃ (78.7 mL, 78.7 mmol, 3.0 eq) at 0° C. The mixture was stirred at room temperature for 15 hours. HPLC showed the reaction was completed. Then the mixture was poured into water and extracted with EtOAc (×3). The combined organic layers were washed with brine, dried over Na₂SO₄, filtered and concentrated to give N-5 (11.0 g). LCMS (ESI, m/z): [M+Na]⁺=462.2.

### Step 5 (Synthesis of Compound N-6)

To a solution of N-5 (10.0 g, 23.0 mmol, 1.0 eq) in THE (120 mL) was added TBAF (25 mL, 25.0 mmol, 1.1 eq). The mixture was stirred at room temperature for 6 hours. HPLC showed the reaction was completed. Then the mixture was added EtOAc, washed with water, brine, dried over Na₂SO₄, filtered and concentrated to give N-6 (3.6 g), which was used in next step without further purification.

### Step 6 (Synthesis of Compound N-7)

To a solution of N-6 (3.6 g, 17.9 mmol, 1.0 eq) in DCM (36 mL) was added TEA (5.4 g, 53.7 mmol, 3.0 eq) and MsCl (3.7 g, 32.2 mmol, 1.8 eq) at 0° C. under N2 atmosphere. The mixture was stirred at room temperature for 6 hours. Then the mixture was washed with water and then brine, dried over Na₂SO₄, filtered and concentrated to give N-7 (4.8 g, crude). LCMS (ESI, m/z): [M+Na]⁺=302.0.

### Step 7 (Synthesis of Compound N-8)

To a solution of N-7 (4.5 g, 16.1 mmol, 1.0 eq) in DMSO (45 mL) was added NaCN (1.2 g, 24.2 mmol, 1.5 eq). The mixture was stirred at 80° C. for 15 hours. LCMS showed completion. Then the mixture was added a saturated aqueous solution of NaHCO₃ and extracted with EtOAc (×3). The combined organic layers were washed with brine, dried over Na₂SO₄, filtered and concentrated to give a residue. The residue was purified by silica gel column chromatography (PE:EtOAc=5:1) to give N-8 (600 mg).

### Step 8 (Synthesis of Compound N-9)

A solution of N-8 (560 mg, 2.7 mmol, 1.0 eq) in concentrated HCl solution (5.6 mL) was stirred at 80° C. for 2 hours. LCMS showed the reaction was completed. Then the mixture was cooled and concentrated to give N-9 (600 mg). LCMS (ESI, m/z): [M+H]⁺=130.2.

### Step 9 (Synthesis of Compound N-10)

To a solution of N-9 (600 mg, 3.6 mmol, 1.0 eq) in MeOH (6 mL) was added SOCl₂ (862 mg, 7.2 mmol, 2.0 eq) at 0° C. The mixture was stirred at room temperature for 15 hours. LCMS showed the reaction was completed. Then the mixture was concentrated to give N-10 (800 mg, crude). LCMS (ESI, m/z): [M+H]⁺=144.1.

### Step 10 (Synthesis of Compound N-11)

To a solution of N-10 (800 mg, 4.5 mmol, 1.0 eq) in DCM (16 mL) was added TEA (1.7 g, 16.8 mmol) and MsCl (960 mg, 8.4 mmol) at 0° C. under N2 atmosphere. The mixture was stirred at room temperature for 15 hours. LCMS showed the reaction was completed. Then the mixture was washed with water and then brine, dried over Na₂SO₄, filtered and concentrated to give N-11 (900 mg, crude).

The relevant characterization data was as follows: LCMS (ESI, m/z): [M+H]⁺=222.1. ¹H NMR (300 MHz, CDCl₃, ppm): δ 4.00-3.88 (m, 1H), 3.75-3.60 (m, 2H), 3.27-3.19 (m, 1H), 2.87 (s, 3H), 2.42-2.33 (m, 1H), 2.00-1.92 (m, 1H), 1.33 (d, J=6.3 Hz, 3H).

### Step 11 (Synthesis of Compound N)

To a solution of N-11 (900 mg, 4.1 mmol, 1.0 eq) in MeOH (6 mL) and H₂O (2 mL) was added LiOH·H₂O (334 mg, 8.1 mmol, 2.0 eq). The mixture was stirred at room temperature for 3 hours. LCMS showed the reaction was completed. The mixture was extracted with DCM (×3) and the aqueous phase was acidified to pH 1, and then extracted with DCM (×3). The combined organic layers were washed with brine, dried over Na₂SO₄, filtered and concentrated to give N (400 mg).

The relevant characterization data was as follows: ¹H NMR (300 MHz, CDCl₃, ppm): δ 4.00-3.88 (m, 1H), 3.75-3.60 (m, 2H), 3.27-3.19 (m, 1H), 2.87 (s, 3H), 2.42-2.33 (m, 1H), 2.00-1.92 (m, 1H), 1.33 (d, J=6.3 Hz, 3H).

### Intermediate O

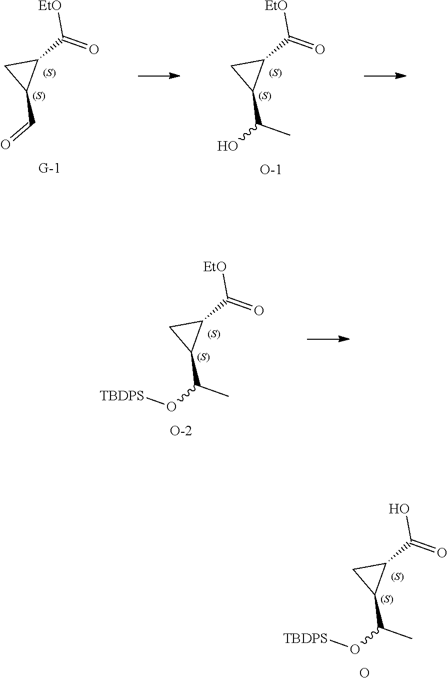

### Step 1 (Synthesis of Compound O-1)

To a solution of G-1 (1.6 g, 11.3 mmol, 1.0 eq) in THE (20 mL) was added MeMgBr (3.8 mL, 11.3 mmol, 1.0 eq, 3M) under argon at −78° C., the mixture was stirred for 2 hours at −78° C. The reaction mixture was quenched with saturated aqueous NH₄Cl solution and extracted with EtOAc (×3), the combined organic layers were washed with brine, dried over Na₂SO₄ and concentrated. The residue was purified by column chromatography on silica gel (PE:EtOAc=5:1) to give the 0-1 (1.3 g).

### Step 2 (Synthesis of Compound O-2)

To a solution of 0-1 (1.0 g, 6.3 mmol, 1.0 eq) in DMF (30 mL) at room temperature were added imidazole (670 mg, 9.5 mmol, 1.5 eq) and TBDPSCl (2.6 g, 9.5 mmol, 1.5 eq). The mixture stirred at room temperature for 12 hours. The reaction mixture was poured into water and extracted with EtOAc, the combined organic layers were washed with brine, dried over Na₂SO₄ and concentrated to afford 0-2 (3.1 g, crude), which was used in next step without further purification.

### Step 3 (Synthesis of Compound O)

To a solution of 0-2 (3.1 g, crude) in MeOH (30 mL), THE (30 mL) and H₂O (15 mL) was added NaOH (345 mg, 9.6 mmol, 1.5 eq) at room temperature. Then the mixture was stirred for 12 hours at same temperature. The mixture was adjusted to pH 3 to 4 with 2 N HCl aqueous solution and the reaction mixture was extracted with DCM:MeOH (10:1, ×3), the combined organic layers were washed with brine, dried over Na₂SO₄ and concentrated. The residue was purified by column chromatography on silica gel (PE:EtOAc=20:1) to give O (1.2 g).

The relevant characterization data was as follows: LCMS (ESI, m/z): [M−H]⁻=366.8; ¹HNMR (300 MHz, CDCl₃, ppm): δ 7.74-7.64 (m, 4H), 7.47-7.36 (m, 6H), 3.58-3.46 (m, 1H), 1.71-1.60 (m, 1H), 1.51-1.44 (m, 1H), 1.18 (d, J=6.0 Hz, 3H), 1.14-1.06 (m, 9H), 0.97-0.75 (m, 2H).

### Intermediate P

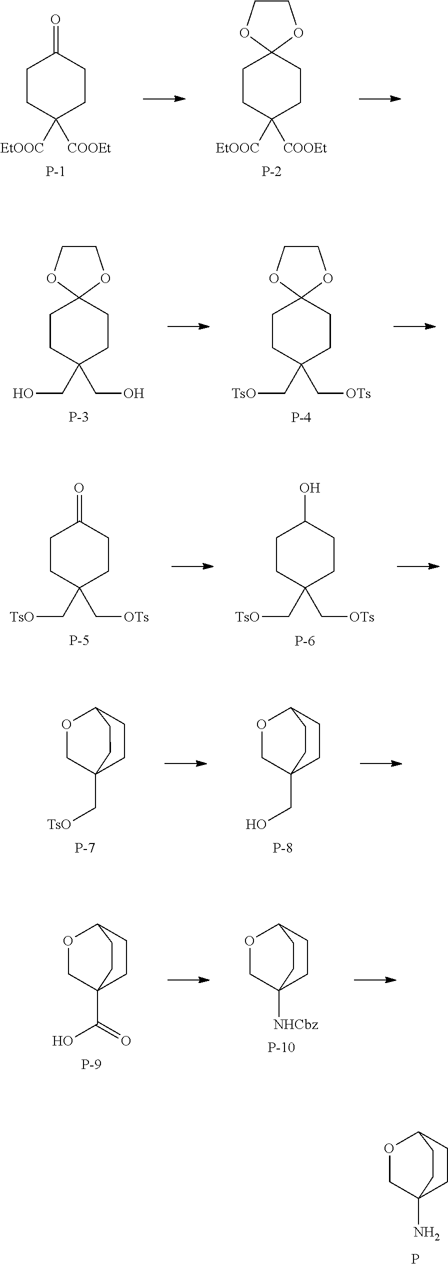

### Step 1 (Synthesis of Compound P-2)

To the solution of P-1 (19.0 g, 78.4 mmol, 1.0 eq) in toluene (190 mL) were added ethylene glycol (5.8 g, 94.1 mmol, 1.2 eq) and p-toluenesulfonic acid hydrate (148 mg, 0.8 mmol, 0.01 eq) and the mixture was heated at reflux for 3 hours via Dean-Stark apparatus. HPLC showed the reaction was completed. Then the mixture was quenched with water. The aqueous phase was extracted with EtOAc (×2). The combined organic phase was washed with aqueous saturate NaHCO₃ solution and brine, dried over Na₂SO₄, filtered and concentrated to give P-2 (22.0 g), which was used directly in next step. LCMS (ESI, m/z): [M+H]⁺=287.2.

### Step 2 (Synthesis of Compound P-3)

Under the atmosphere of N2, to a solution of LiAlH₄ (8.8 g, 230.5 mmol, 3.0 eq) in THE (180 mL) was added the solution of P-2 (22.0 g, 76.8 mmol, 1.0 eq) in THE (40 mL) dropwise over a period of 1 hour while maintaining the internal temperature at −30° C. After addition, the mixture was allowed to warm to 0° C. and stirred for 2 hours. HPLC showed the reaction was completed. The mixture was quenched with water (8.8 mL). Then aqueous NaOH solution (15%, 8.8 mL) and water (26.4 mL) were added dropwise. The aqueous phase was extracted with DCM:i-PrOH (3:1, ×2). The combined organic phase was washed with brine, dried over Na₂SO₄, concentrated to give crude product. The residue was purified by silica gel chromatography (PE:EtOAc=2:1) to give P-3 (12.8 g), which was used in next step without further purification. LCMS (ESI, m/z): [M+H]⁺=203.0.

### Step 3 (Synthesis of Compound P-4)

To a solution of P-3 (12.4 g, 61.3 mmol, 1.0 eq) in pyridine (74.4 mL) was added p-toluenesulfonyl chloride (35.1 g, 183.9 mmol, 3.0 eq) at 0° C., the resulting mixture was stirred at room temperature overnight. LCMS showed the completion of the reaction. The mixture was diluted with EtOAc, washed with aqueous citric acid solution (10%, ×4) and brine, dried over Na₂SO₄, concentrated to give crude product which was purified by silica gel chromatography (PE:EtOAc=5:1) to give P-4 (29.6 g).

### Step 4 (Synthesis of Compound P-5)

A mixture of P-4 (14.8 g, 28.2 mmol, 1.0 eq), hydrochloric acid (1N, 113 mL, 113 mmol, 4.0 eq) and tetrahydrofuran (150 mL) was heated under reflux for 2 hours. the reaction was completed. The mixture was cooled to room temperature, the aqueous phase was extracted with EtOAc (×2). The combined organic phase was washed with NaHCO₃ and then brine, dried over Na₂SO₄, filtered and concentrated to give P-5 (14.5 g).

### Step 5 (Synthesis of Compound P-6)

To a mixture of P-5 (10.0 g, 21.4 mmol, 1.0 eq) in DCM (100 mL) was added a solution of diisobutyl aluminium hydride (17 mL, 25.7 mmol, 1.2 eq) in hexanes at −40° C. The mixture was allowed to warm to −20° C., another diisobutyl aluminium hydride (2.5 mL, 3.8 mmol, 0.2 eq) was added and stirred for 2 hours. HPLC showed the reaction was completed. The reaction was warm to 0° C., quenched with Rochelle salt solution and extracted with DCM (×3). The combined organic layers was dried over Na₂SO₄, filtered and concentrated and purified by silica gel chromatography (PE:EtOAc=5:1) to give P-6 (8.9 g).

### Step 5 (Synthesis of Compound P-7)

To the mixture of P-6 (12.0 g, 25.6 mmol, 1.0 eq) in 1,2-dimethoxyethane (120.0 mL) was added sodium hydride (3.1 g, 60% in mineral oil, 76.8 mmol, 3.0 eq) and heated at reflux for 1.5 hours. HPLC showed the reaction was completed. The reaction was cooled to 0° C. and quenched with aqueous saturated ammonium chloride solution and extracted with EtOAc (×3). The combined organic phase was washed with H₂O and brine, dried over Na₂SO₄, filtered and concentrated, the residue was purified by silica gel chromatography (PE:EtOAc=5:1) to give P-7 (5.8 g).

### Step 7 (Synthesis of Compound P-8)

A mixture of P-7 (5.0 g, 16.9 mmol, 1.0 eq), potassium acetate (5.0 g, 50.6 mmol, 3.0 eq) in DMF (50 mL) was heated at 110° C. for 1 hour. HPLC showed the reaction was completed. Then the mixture was quenched with water, extracted with EtOAc. The combined organic phase was washed with H₂O and then brine, dried over Na₂SO₄, filtered and concentrated to give crude product, which was dissolved in H₂O (30 mL) and methanol (25 mL). K₂CO₃ (11.6 g, 84.3 mmol, 5.0 eq) was added and the mixture was heated to 50° C. for 5 hours, HPLC showed the reaction was completed. The mixture was treated with water and cooled to room temperature, extracted with EtOAc. The combined organic phase was washed with H₂O and then brine, dried over Na₂SO₄, filtered and concentrated to give P-8 (2.0 g).

### Step 8 (Synthesis of Compound P-9)

To a mixture of P-8 (2.84 g, 20.0 mmol, 1.0 eq) in DMF (28 mL) was added Jones reagent (35 mL, 70.0 mmol, 3.5 eq) at 0° C., warmed to 25° C. and stirred for 1.5 hours. Cooled to 10° C., the mixture was quenched with water, extracted with EtOAc (×2). The combined organic phase was washed with H₂O and then brine, dried over Na₂SO₄, filtered and concentrated, the residue was purified by silica gel chromatography (PE:EtOAc=3:1) to give P-9 (2.2 g). LCMS (ESI, m/z): [M−H]⁻=155.0.

### Step 9 (Synthesis of Compound P-10)

To the mixture of P-9 (1.0 g, 6.4 mmol, 1.0 eq) in toluene (20 mL) was added DIEA (1.7 g, 12.8 mmol, 2.0 eq) and DPPA (2.1 g, 7.7 mmol, 1.2 eq), the mixture was heated at reflux for 2 hours. The mixture was cooled to 60° C., and benzyl alcohol (1.4 g, 12.8 mmol, 2.0 eq) was added and stirred at reflux overnight. LCMS showed the reaction was completed. After cooling, the mixture was treated with water and extracted with EtOAc (×2). The combined organic phase was washed with H₂O and then brine, dried over Na₂SO₄, filtered and concentrated, the residue was purified by silica gel chromatography (PE EtOAc=5:1) to give P-10 (1.0 g).

The relevant characterization data was as follows: LCMS (ESI, m/z): [M+H]⁺=262.0.

### Step 10 (Synthesis of Compound P)

To the solution of P-10 (1.3 g, 5.0 mmol, 1.0 eq) in methanol (13 mL) was added Pd/C (130 mg, 10% wt.). The reaction was stirred under the atmosphere of hydrogen overnight. LCMS showed the reaction was completed. The reaction mixture was filtered through a pad of Celite and filtrate was concentrated to afford P (520 mg).

The relevant characterization data was as follows: ¹HNMR (300 MHz, METHANOL-d₄, ppm): δ 3.84-3.80 (m, 3H), 2.19-2.09 (m, 2H), 1.99-1.80 (m, 6H).

### Intermediate Q

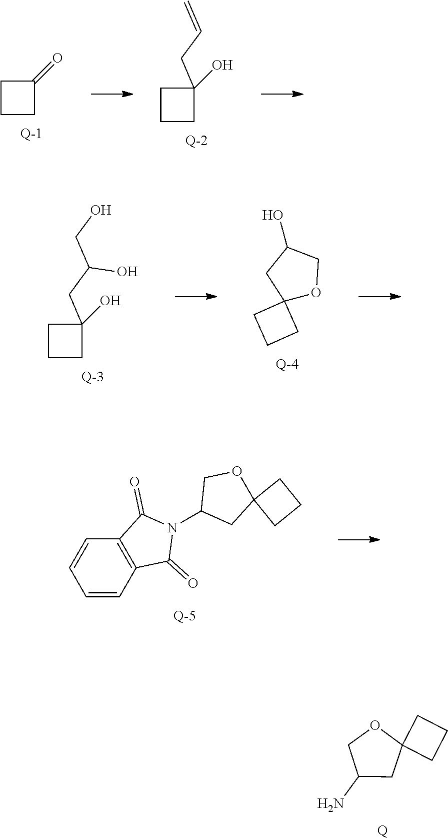

### Step 1 (Synthesis of Compound Q-2)

To a solution of allylmagnesium bromide in THF (0.7 M, 917.0 mL, 642.0 mol, 3.0 eq) in THE (300 mL) was added a solution of Q-1 (15.0 g, 214.0 mol, 1.0 eq) in THE (225 mL) dropwise at 0° C. under N2. The reaction mixture was stirred at 25° C. for 4 hours. After cooling to 0° C., the reaction was quenched by the addition of saturated NH₄Cl solution, extracted with EtOAc (×4). The combined organic layers were washed with brine, dried over Na₂SO₄ and concentrated to give Q-2 (26.0 g, crude), which was used directly in next step without further purification.

### Step 2 (Synthesis of Compound Q-3)

To a mixture of Q-2 (26.0 g, 231.7 mmol, 1.0 eq) and 4-methylmorpholine N-oxide (209.1 g, 1.78 mol, 7.7 eq) in THE (390 mL) was added dropwise a solution of OsO₄ (2.5% in tert-butanol, 118.0 g, 11.6 mmol, 0.05 eq). The reaction mixture was stirred at 25° C. for 12 hours. TLC showed the completion of the reaction. The mixture was cooled to 0° C. and quenched by the addition of aqueous saturated Na₂S₂O₃ solution, then stirred at room temperature for 3 hours. The reaction mixture was extracted with 1-butanol (×3). The combined organic phase was washed with brine, dried over Na₂SO₄, filtered and concentrated to give a residue which was purified by silica gel chromatography (DCM:MeOH=30:1) to give Q-3 (13.0 g).

### Step 3 (Synthesis of Compound Q-4)

To a solution of Q-3 (13.0 g, 88.9 mmol, 1.0 eq) in DCM (890 mL) were added 4-methylbenzenesulfonyl chloride (18.7 g, 97.8 mmol, 1.1 eq), Bu₂SnO (1.1 g, 4.5 mmol, 0.05 eq) and TEA (9.9 g, 88.9 mmol, 1.0 eq). The mixture was stirred at 40° C. for 16 hours. Then the mixture was concentrated to give a residue which was purified by silica gel chromatography (PE:EtOAc=1:1) to give Q-4 (7.5 g).

### Step 4 (Synthesis of Compound Q-5)

To a solution of Q-4 (7.5 g, 58.5 mmol, 1.0 eq) in THF (100 mL) were added isoindoline-1,3-dione (12.9 g, 87.8 mmol, 1.5 eq), PPh₃ (16.9 g, 64.4 mmol, 1.1 eq), diisopropyl azodicarbolate (14.2 g, 70.2 mmol, 1.2 eq) under N2. The reaction mixture was stirred at room temperature for 4 hours. The reaction was quenched with water and extracted with ethyl acetate (×2). The combined organic phase was washed with brine, dried over Na₂SO₄, filtered and concentrated to give a residue which was purified by silica gel chromatography (PE:EtOAc=5:1) to give Q-5 (8.3 g). LCMS (ESI, m/z): [M+H]⁺=258.0.

### Step 5 (Synthesis of Compound Q)

To a solution of Q-4 (8.3 g, 32.3 mmol, 1.0 eq) in MeOH (110 mL) was added hydrazine hydrate (98%, 3.29 g, 64.5 mmol, 2.0 eq). The reaction mixture was heated to 40° C. for 4 hours. The reaction was cooled to room temperature and diluted with DCM:MeOH (10:1). Then the reaction mixture was filtered, and the filtrate was concentrated to give a residue which was purified by silica gel chromatography (DCM:MeOH=30:1) to give Q (2.0 g).

The relevant characterization data was as follows: ¹HNMR (400 MHz, CDCl₃, ppm): δ 3.92-3.88 (m, 1H), 3.58-3.55 (m, 1H), 3.47-3.43 (m, 1H), 2.35-2.10 (m, 4H), 2.03-1.95 (m, 1H), 1.79-1.62 (m, 2H), 1.55-1.45 (m, 1H).

### Intermediate R

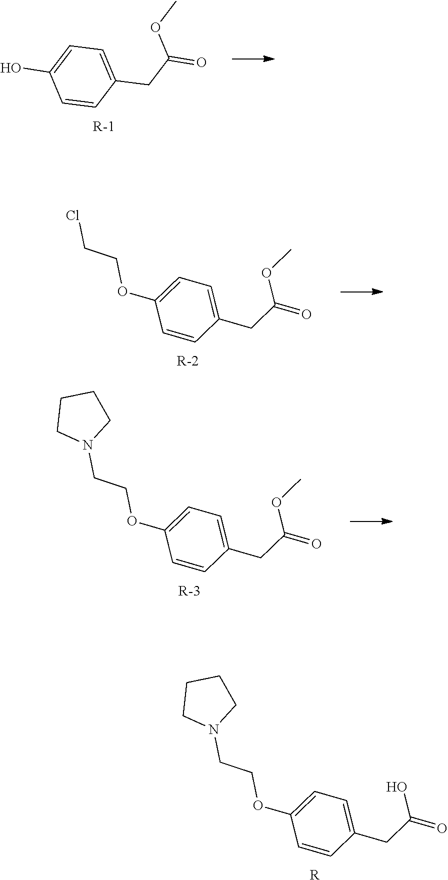

### Step 1 (Synthesis of Compound R-2)

To a solution of R-1 (5240 mg, 31.5 mmol) in acetonitrile (100 mL) were added potassium carbonate (13100 mg, 94.8 mmol) and 1-bromo-2-chloroethane (13600 mg, 94.8 mmol). The reaction was stirred at 85° C. for 48 hours. The mixture was cooled, filtered and concentrated. The residue was purified by chromatography on a silica gel column (petroleum ether to petroleum ether/ethyl acetate=4/1) to give R-2 (3700 mg). LCMS (ESI, m/z): [M+H]⁺=229.0.

### Step 2 (Synthesis of Compound R-3)

To a solution of R-2 (3500 mg, 15.3 mmol) in dimethylformamide (100 mL) were added pyrrolidine (1630 mg, 22.9 mmol), potassium carbonate (3175 mg, 23.0 mmol) and potassium iodide (3812 mg, 23.0 mmol). The reaction was stirred at 100° C. for 8 hours. HPLC showed the reaction was completed. The mixture was cooled and filtered. The filtrate was extracted with ethyl acetate and washed with water. The organic phase was concentrated and purified by chromatography on a silica gel column (dichloromethane to dichloromethane/methanol=10/1) to give R-3 (2400 mg). LCMS (ESI, m/z): [M+H]⁺=264.2.

### Step 3 (Synthesis of Compound R)

To a solution of R-3 (1000 mg, 3.8 mmol) in methanol (10 mL)/water (10 mL) was added sodium hydroxide (455 mg, 11.4 mmol). The reaction was stirred at room temperature for 16 hours. The mixture was concentrated and purified by prep-HPLC (acetonitrile/0.05% TFA in water: 5%˜50%) to give R (600 mg). LCMS (ESI, m/z): [M+H]⁺=250.2.

### Intermediate S

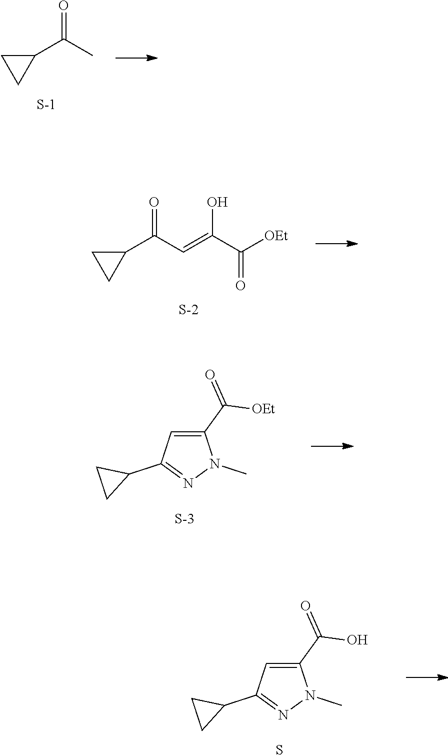

### Step 1 (Synthesis of Compound S-2)

Under the atmosphere of nitrogen, Na wire (2.7 g, 118.9 mmol, 1.0 eq) was added slowly into EtOH (100 mL), the mixture was stirred at room temperature for 2 hours until Na was disappeared. The reaction mixture was cooled to 0° C., then S-1 (10.0 g, 118.9 mmol, 1.0 eq) and diethyl oxalate (17.4 g, 118.9 mmol, 1.0 eq) were added. The reaction mixture was stirred at 25° C. for 1 hour and heated at 80° C. for 2 hours. The mixture was cooled to 0° C., treated with water and acidified with aq. HCl solution (2N) to pH=4, extracted with EtOAc (×3). The combined organic phase was washed with brine, dried over Na₂SO₄ and concentrated to give a residue which was purified by silica gel chromatography (eluted with PE:EtOAc=40:1) to give S-2 (16.0 g). LCMS (ESI, m/z): [M+H]⁺=185.1

### Step 2 (Synthesis of Compound S-3)

To a solution of S-2 (10.0 g, 54.3 mmol, 1.0 eq) in EtOH (100 mL) was added dropwise aqueous methylhydrazine solution (40%, 12.5 g, 108.6 mmol, 2.0 eq) at 0° C. The reaction mixture was stirred at 0° C. for 1 hour and stirred at 25° C. for 12 hours. The reaction mixture was concentrated, diluted with water and extracted with EtOAc (×2). The combined organic phase was washed with brine, dried over Na₂SO₄ and concentrated to give a residue. The residue was purified by silica gel chromatography (eluted with PE EtOAc=20:1) to give S-3 (6.4 g). LCMS (ESI, m/z): [M+H]⁺=195.2.

### Step 3 (Synthesis of Compound S)

To a solution of S-3 (6.4 g, 33.0 mmol, 1.0 eq) in THF/H₂O (96 mL/32 mL) was added LiOH (1.2 g, 49.0 mmol, 1.5 eq) and stirred at 25° C. for 12 hours. The reaction mixture was acidified with the solution of HCl in EtOAc (4N, 15 mL) to pH=3 and concentrated to give a residue. The residue was purified by silica gel chromatography (DCM:MeOH=10:1) to give S (5.2 g).

The relevant characterization data was as follows: LCMS (ESI, m/z): [M+H]⁺=167.2, ¹HNMR (300 MHz, DMSO-d₆, ppm): δ 6.49 (s, 1H), 3.95 (s, 3H), 1.87-1.80 (m, 1H), 0.89-0.81 (m, 2H), 0.65-0.60 (m, 2H).

### Intermediate T

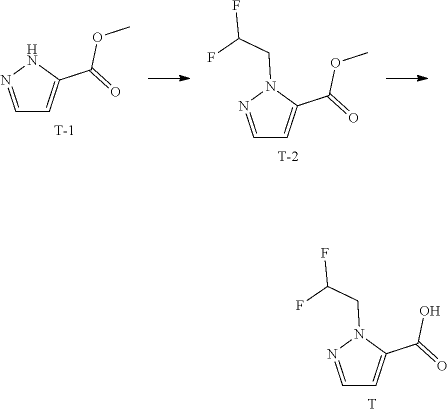

### Step 1 (Synthesis of Compound T-2)

To the solution of methyl T-1 (2.5 g, 19.8 mmol) in acetonitrile (100 mL) were added 2,2-difluoroethyl trifluoromethanesulfonate (5.1 g, 23.8 mmol) and Cs₂CO₃ (9.7 g, 29.7 mmol) at room temperature under N2, then stirred at 60° C. for 3 hours. Then the reaction was diluted with EtOAc, the organic layer was washed with H₂O, aq. NaCl, dried over sodium sulfate and concentrated. The residue was purified by column chromatography on silica gel (petroleum ether to petroleum ether/ethyl acetate=1/2) to give T-2 (1.5 g).

The relevant characterization data was as follows: LCMS (ESI, m/z): [M+H]⁺=191.0, ¹HNMR (400 MHz, CDCl₃, ppm): δ 7.60 (d, J=2.0 Hz, 1H), 6.92 (d, J=2.0 Hz, 1H), 6.31-6.01 (t, J=56.0 Hz, 1H), 5.04-4.97 (m, 2H), 3.92 (s, 3H).

### Step 2 (Synthesis of Compound T)

To the solution of T-2 (480 mg, 2.52 mmol) in THE (8 mL) and H₂O (8 mL) was added LiOH·H₂O (317.8 mg, 7.57 mmol) at 0° C. and stirred at room temperature for 16 hours. Then the reaction was adjusted with 1N HCl to pH=4, diluted with EtOAc, the organic layer was then washed with H₂O, aq. NaCl, dried over sodium sulfate and concentrated to give T (400 mg). LCMS (ESI, m/z): [M+H]⁺=177.0

### Intermediate U

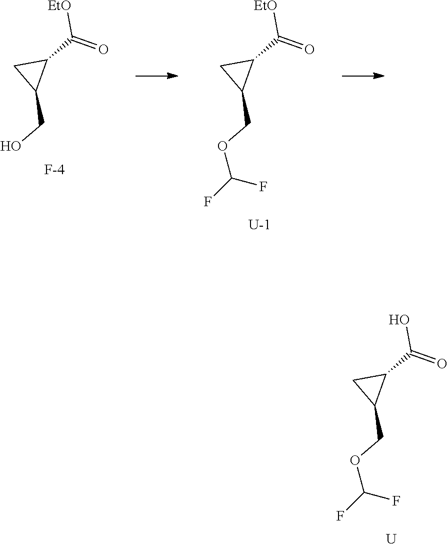

### Step 1 (Synthesis of Compound U-1)

To a solution of F-4 (2.0 g, 13.9 mmol, 1.0 eq) in DCM (10 mL) was added (bromodifluoromethyl)trimethylsilane (5.6 g, 27.7 mmol, 2.0 eq) and KOAc (5.4 g, 55.5 mmol, 4.0 eq) in H₂O (10 mL). The reaction mixture was stirred at room temperature for 6 hours. TLC showed the reaction was completed. Then the mixture was partitioned. The aqueous phase was extracted with DCM. The combined organic layers were washed with brine, dried over Na₂SO₄, filtered and concentrated to give U-1 (2 g), which was used directly in next step.

### Step 2 (Synthesis of Compound U)

To a solution of U-1 (1.0 g, 5.15 mmol, 1.0 eq) in MeOH (20 mL) was added LiOH·H₂O (238 mg, 5.66 mmol, 1.1 eq) in H₂O (5 mL). The reaction mixture was stirred at 40° C. for 1 hour. TLC showed the reaction was completed. Then the mixture was extracted with DCM (×3). The aqueous phase was acidified to pH˜1 and then extracted with DCM (×3). The combined organic layers were washed with brine, dried over Na₂SO₄, filtered and concentrated at 30° C. to give U (500 mg, crude), which was used directly in next step.

The relevant characterization data was as follows: ¹HNMR (300 MHz, CDCl₃, ppm): δ 6.21 (t, J=74.1 Hz, 1H), 3.88 (dd, J=10.8, 6.0 Hz, 1H), 3.73-3.62 (m, 1H), 1.90-1.79 (m, 1H), 1.68-1.59 (m, 1H), 1.37-1.27 (m, 1H), 1.05-0.95 (m, 1H).

### Intermediate V

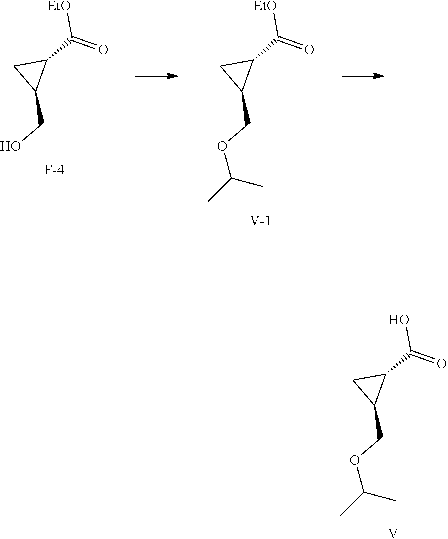

### Step 1 (Synthesis of Compound V-1)

To a suspension of F-4 (1.0 g, 6.9 mmol, 1.0 eq) and AgOTf (2.7 g, 10.4 mmol, 1.5 eq) in DCM (10 mL), was added 2-iodopropane (2.4 g, 13.9 mmol, 2.0 eq) dropwise at 0° C., the reaction mixture was allowed to warm to room temperature and stirred overnight. TLC showed the reaction was completed. Filtered through a Celite pad, the filtrate was concentrated, the residue was purified on silica gel column chromatography (PE:EA=20:1 to 5:1) to give V-1 (550 mg).

### Step 2 (Synthesis of Compound V)

To a solution of V-1 (500 mg, 2.7 mmol, 1.0 eq) in MeOH (5 mL) was added NaOH (120 mg, 3.0 mmol, 1.1 eq) in water (2 mL), the reaction mixture was stirred at room temperature overnight before it was concentrated. The residue was diluted with THE (5 mL), HCl in EtOAc (3 M, 3 mL) was added dropwise at 0° C. and stirred for 30 minutes. The mixture was filtered and concentrated, the residue was purified on silica gel column chromatography (PE:EtOAc=20:1 to 2:1) to give V (400 mg).

¹HNMR (400 MHz, CDCl₃, ppm): δ 3.61-3.53 (m, 1H), 3.44-3.29 (m, 2H), 1.79-1.72 (m, 1H), 1.57-1.52 (m, 1H), 1.28-1.23 (m, 1H), 1.14 (d, J=6.0 Hz, 6H), 0.96-0.90 (m, 1H).

### Intermediate W

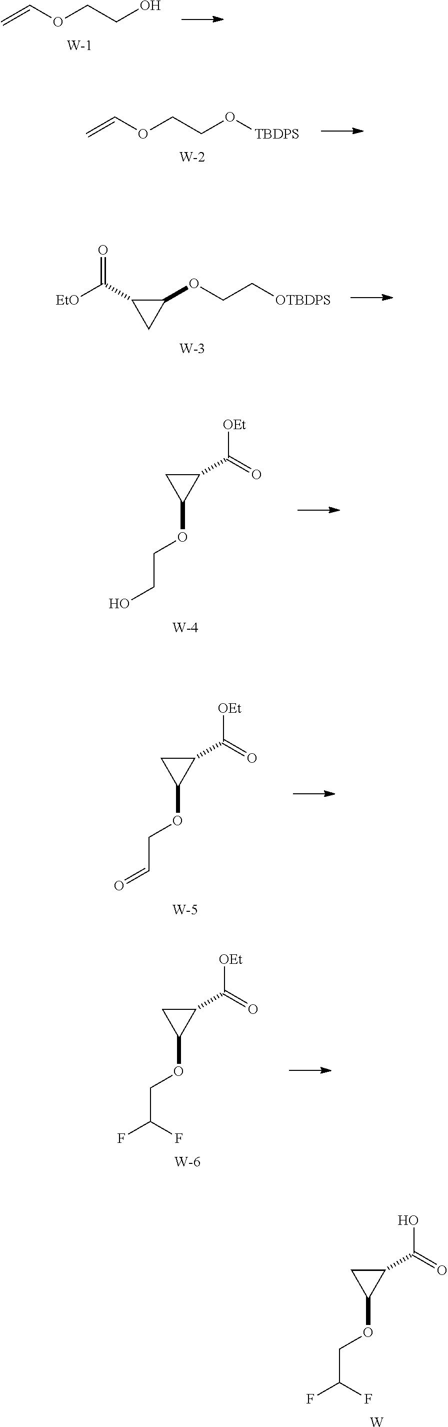

### Step 1 (Synthesis of Compound W-2)

To a solution of W-1 (5.0 g, 56.747 mmol) and imidazole (7.7 g, 113.494 mmol) in N, N-dimethylformamide (50 mL) was added tert-butylchlorodiphenylsilane (22.135 mL, 85.121 mmol) dropwise at 0˜5° C. After addition, the reaction solution was warmed to 25° C. and stirred for 64 hours. The reaction mixture was diluted with water and extracted with ethyl acetate. The combined organic layers were washed with water and then brine, dried over anhydrous sodium sulphate, filtered and concentrated in vacuo. The residue was purified by silica gel chromatography (PE:EtOAc 93:7) to afford W-2 (14.4 g).

### Step 2 (Synthesis of Compound W-3)

To a stirred solution of W-2 (13.40 g, 41.0 mmol) in dichloromethane (260 mL) was added rhodium (II) acetate dimer (0.3 g, 0.71 mmol) under nitrogen atmosphere, then ethyl 2-diazoacetate (9.37 g, 82.1 mmol) was added dropwise. The resulting mixture was stirred at 25° C. for 20 hours. The reaction mixture was filtered and the filtrate was concentrated in vacuo. The residue was purified by silica gel chromatography (PE:EtOAc 92.5:7.5) to afford W-3 (7.6 g).

### Step 3 (Synthesis of Compound W-4)

To a stirred solution of W-3 (3.0 g, 7.3 mmol) in tetrahydrofuran (30 mL) was added tetrabutylammonium fluoride solution (14.5 mL, 14.5 mmol, 1.0 M). The reaction mixture was stirred at 25° C. for 1.5 hours. The mixture was dissolved in water and extracted with ethyl acetate (×4). The combined organic layers were washed with water and then brine, dried over anhydrous sodium sulphate, filtered and concentrated in vacuo. The residue was purified by silica gel chromatography (PE:EtOAc=2:3) to afford W-4 (960 mg).

### Step 4 (Synthesis of Compound W-5)

To a solution of W-4 (960 mg, 5.51 mmol) in dichloromethane (20 mL) was added Dess-martin periodinane (3506.1 mg, 8.27 mmol). The mixture was stirred at 25° C. for 15 hours. The reaction mixture was quenched with saturated sodium bicarbonate solution (30 mL) and saturated sodium thiosulfate solution (30 mL), stirred at 30 minutes, then extracted with ethyl acetate (×3). The combined organic layers were washed with water and then brine, dried over anhydrous sodium sulphate, filtered and concentrated in vacuo. The residue was purified by silica gel chromatography (PE:EtOAc=1:1) to afford W-5 (340 mg).

### Step 5 (Synthesis of Compound W-6)

A solution of W-5 (340 mg, 1.98 mmol) in dichloromethane (5 mL) was purged with nitrogen three times, then cooled to −20° C.˜15° C., diethylaminosulphur trifluoride (1591.5 mg, 9.87 mmol) was added dropwise. After addition, the resulting mixture was allowed to warmed to 25° C. and stirred for 17 hours. The reaction was quenched with saturated solution of sodium bicarbonate, diluted with water and extracted with ethyl acetate (×3). The combined organic layers were washed with brine, dried over anhydrous sodium sulphate, filtered and concentrated in vacuo. The residue was purified by silica gel chromatography (PE:EtOAc=4:1) to afford W-6 (100 mg).

### Step 6 (Synthesis of Compound W)

To a stirred solution of W-5 (100 mg, 0.515 mmol) in tetrahydrofuran (1.5 mL) and water (1.5 mL) was added lithium hydroxide hydrate (32.4 mg, 0.772 mmol). The mixture was stirred at 25° C. for 2 hours. The reaction mixture was adjusted pH=1˜2 with 1N hydrochloric acid solution and extracted with ethyl acetate (×3). The combined organic layers were washed with brine, dried over anhydrous sodium sulphate, filtered and concentrated in vacuo to afford W (71 mg), which was used directly in next step.

The relevant characterization data was as follows: ¹H NMR (400 MHz, DMSO-d₆, ppm): δ 12.26 (s, 1H), 6.29-6.01 (t, J=52.0 Hz, 1H), 3.85-3.68 (m, 3H), 1.72-1.69 (m, 1H), 1.25-1.05 (m, 2H).

### Intermediate X

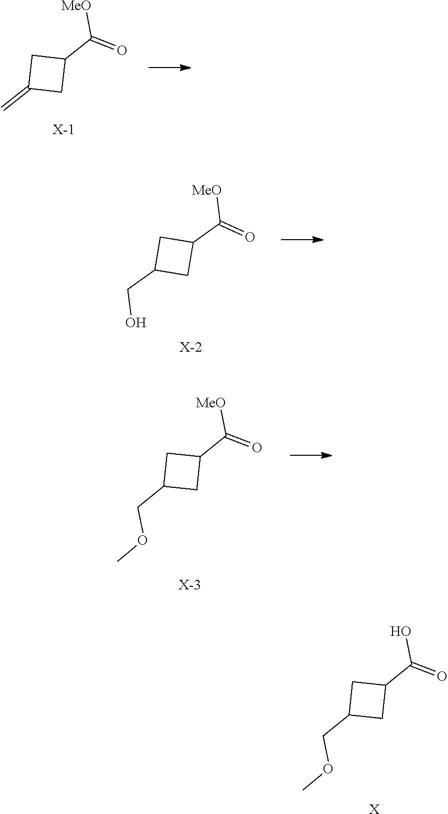

### Step 1 (Synthesis of Compound X-2)

To a stirred solution of methyl X-1 (2.0 g, 15.8 mmol) in anhydrous tetrahydrofuran (20 mL) urged with nitrogen three times was added borane dimethyl sulfide complex (7.926 mL, 15.8 mmol) dropwise at −15° C. After addition, the ice bath was removed and the temperature was allowed to warmed to 25° C. and stirred for 4 hours. Then cooled to −15° C., 3.0 M sodium hydroxide (2.1 mL, 6.3 mmol) and 30% hydrogen peroxide (1.8 g, 15.8 mmol) were added. The resulted mixture was stirred at 25° C. for 2 hours. The reaction mixture was quenched with 10% sodium bisulfate solution and extracted with ethyl acetate (×5). The combined organic layers were washed with brine, dried over anhydrous sodium sulphate, filtered and concentrated in vacuo. The residue was purified by silica gel chromatography (PE:EtOAc=3:7) to afford X-2 (1200 mg).

### Step 2 (Synthesis of Compound X-3)

To a stirred solution of X-2 (800 mg, 5.55 mmol) in N, N-dimethylformamide (10 mL) was added silver oxide (3857.8 mg, 16.65 mmol), followed by iodomethane (2362.88 mg, 16.65 mmol). The resulted mixture was stirred at 25° C. for 18 hours. The reaction mixture was filtered and the filtrate was diluted with water, extracted with ethyl acetate (×3). The combined organic layers were washed with water and then brine, dried over anhydrous sodium sulphate, filtered and concentrated in vacuo. The residue was purified by silica gel chromatography (PE:EtOAc=4:1) to afford X-3 (510 mg).

### Step 3 (Synthesis of Compound X)

To a stirred solution of X-3 (510 mg, 3.22 mmol) in tetrahydrofuran (5 mL) and water (5 mL) was added lithium hydroxide hydrate (202.9 mg, 4.84 mmol). The mixture was stirred at 25° C. for 2 hours. The reaction mixture was adjusted pH=1˜2 with 1N hydrochloric acid solution and extracted with ethyl acetate (×3). The combined organic layers were washed with brine, dried over anhydrous sodium sulphate, filtered and concentrated in vacuo to afford X (400 mg).

The relevant characterization data was as follows: ¹H NMR (400 MHz, DMSO-d₆, ppm): δ 12.02 (s, 1H), 3.34-3.32 (d, J=6.8 Hz, 1H), 3.24-3.21 (m, 4H), 2.99-2.88 (m, 1H), 2.45-2.40 (m, 1H), 2.39-2.11 (m, 2H), 1.94-1.81 (m, 2H).

### Intermediate Y

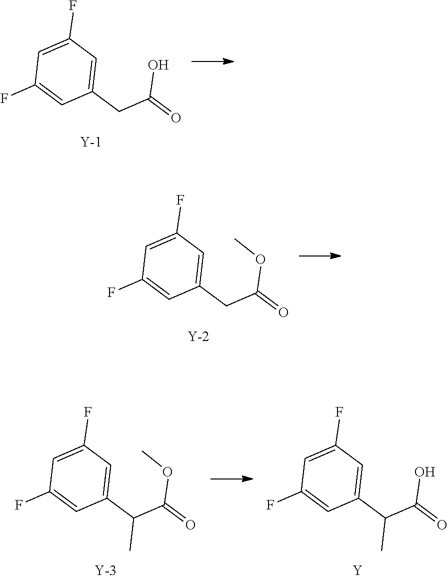

### Step 1 (Synthesis of Compound Y-2)

To a solution of Y-1 (4.5 g, 26.143 mmol) in MeOH (50 mL) was added H₂SO₄ (0.3 mL, 5.628 mmol), and the reaction was stirred at room temperature for 3 hours. The reaction was diluted with EtOAc and water. The organic layer was collected, washed with further saturated NaCl solution, dried over sodium sulphate, filtered and concentrated under vacuum to give Y-2 (4.7 g).

### Step 2 (Synthesis of Compound Y-3)

To a stirred solution of Y-2 (4.2 g, 22.6 mmol) in THF (50 mL) was added lithium diisopropylamide (1M in THF, 27.1 mmol) at 0° C. After 30 mins, CH₃I (4.8 g, 33.8 mmol) was added, and the reaction was stirred at room temperature for 3 hours. The reaction mixture was diluted with EtOAc and saturated NH₄Cl solution. The organic layer was collected, washed with further saturated NaCl solution, dried with sodium sulphate, filtered and concentrated under vacuum to give the title compound Y-3 (4.1 g).

### Step 3 (Synthesis of Compound Y)

To a solution of Y-2 (4.1 g, 20.5 mmol) in THF (15 mL) was added H₂O (15 mL), LiOH·H₂O (0.9 g, 20.5 mmol), and the reaction was stirred at room temperature for 3 hours. The residue was dissolved in EtOAc and 1N HCl solution was added to adjust pH=3, further washed with water. The organic layer was collected, dried over sodium sulphate, filtered and concentrated under vacuum to give Y (3.5 g), which was used in next step. LCMS (ESI, m/z): [M+H]⁺=187.2

### Intermediate Z

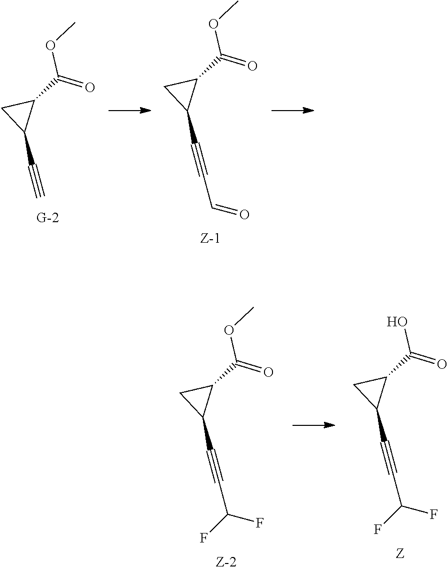

### Step 1 (Synthesis of Compound Z-1)

To a solution of G-2 (800 mg, 6.4 mmol, 1.0 eq) in THF (16 mL) was added n-BuLi (2.4 M, 3.0 mL, 7.1 mmol, 1.1 eq) dropwise at −78° C., the reaction mixture was stirred at −78° C. for 30 minutes. Then methyl formate (464 mg, 7.7 mmol, 1.2 eq) was added dropwise, after addition, the reaction mixture was stirred at −78° C. for 1 hour. TLC showed the reaction was completed. The reaction was quenched with NH₄Cl solution at −20° C., extracted with MTBE (×3), washed with brine, dried over Na₂SO₄, filtered, and concentrated. The residue was purified on silica gel column chromatography (PE:MTBE=20:1) to give Z-1 (300 mg).

### Step 2 (Synthesis of Compound Z-2)

Under N2, to a solution of Z-1 (260 mg, 1.7 mmol, 1.0 eq) in DCM (4 mL) was added DAST (826 mg, 5.1 mmol, 3.0 eq) dropwise at −60° C. The reaction was stirred at room temperature for 1 hour. TLC showed the reaction was completed. The mixture was cooled to 0° C., quenched with aqueous saturated NaHCO₃ solution, extracted with DCM (×3), organic phase was washed with water, brine, dried over Na₂SO₄, filtered and concentrated in vacuo to give a residue which was purified on silica gel column chromatography (PE MTBE=20:1) to give Z-2 (100 mg).

¹HNMR (400 MHz, CDCl₃, ppm): δ 6.13 (t, J=56.0, 0.8 Hz, 1H), 3.72 (s, 1H), 2.06-1.91 (m, 2H), 1.49-1.44 (m, 1H), 1.32-1.26 (m, 1H).

### Step 3 (Synthesis of Compound Z)

To a solution of Z-2 (100 mg, 0.57 mmol, 1.0 eq) in THE (3 mL) and H₂O (3 mL) was added LiOH·H₂O (36 mg, 0.86 mmol, 1.5 eq). The reaction mixture was stirred at room temperature for 1.5 hours. HPLC showed the reaction was completed. The pH of the mixture was adjusted to 1 with 1N HCl. The mixture was extracted with EtOAc (×3), washed with brine, dried over Na₂SO₄, filtered and concentrated in vacuo to give Z (80 mg). LCMS (ESI, m/z): [M−H]⁻=159.1.

### Intermediate AA

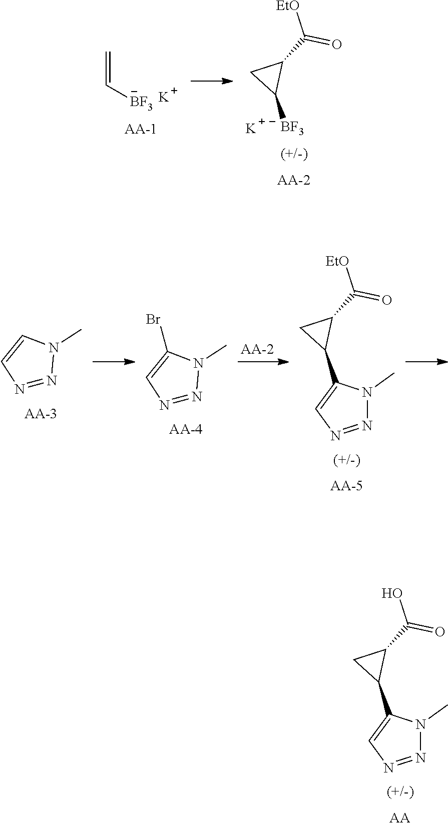

### Step 1 (Synthesis of Compound AA-2

Under nitrogen atmosphere, to a solution of potassium vinyltrifluoroborate (20 g, 149.3 mmol) in THE (200 mL) was added Pd(OAc)₂ (335 mg, 1.5 mmol) and then a solution of ethyl diazoacetate (19.8 g, 173.2 mmol) in THE (20 mL) was added dropwise at 35° C. The reaction mixture was stirred at 35° C. for 15 hours. Then to the mixture was added heptane (500 mL), and stirred at room temperature for 30 minutes. The precipitate was formed at −18° C. and collected by filtration. To the filtrate was added activated carbon (20 g). The mixture was stirred at room temperature for 30 minutes. Filtered, the filter cake was washed with EtOAc (×3). The filtrate was concentrated under reduced pressure to give a residue which was dissolved in EtOH (200 mL), and stirred at 50° C. for 30 minutes. The mixture was allowed to cool down to room temperature. The precipitated solids were collected by filtration and washed with EtOH. The filtrate was concentrated under reduced pressure to give AA-2 (15.0 g).

The relevant characterization data was as follows: ¹H NMR (300 MHz, DMSO-d₆, ppm): δ 3.96 (q, J=7.2 Hz, 2H), 1.15 (t, J=7.2 Hz, 3H), 1.12-1.05 (m, 1H), 0.66-0.59 (m, 1H), 0.52-0.46 (m, 1H), −0.01-0.12 (m, 1H).

### Step 2 (Synthesis of Compound AA-4)

To a solution of AA-3 (5.0 g, 60.2 mmol) in THE (100 mL) was added n-BuLi (27.6 mL, 66.2 mmol, 2.4 N in hexane) dropwise at −78° C. under nitrogen atmosphere. The reaction mixture was stirred at −78° C. for 30 minutes. Then Br₂ (9.6 g, 60.2 mmol) was added dropwise and the mixture was stirred for 30 minutes at −78° C. HPLC showed the reaction was completed. The reaction was quenched with aqueous ammonium chloride solution at −78° C., and then the mixture was extracted with EtOAc (×3). The combined organic extracts were washed with aqueous sodium thiosulfate solution and then brine, dried over anhydrous Na₂SO₄, and concentrated under vacuum to give AA-4 (9.2 g). LCMS (ESI, m/z): [M+H]⁺=162.0.

### Step 3 (Synthesis of Compound AA-5)

To a solution of AA-4 (1.5 g, 9.3 mmol) in toluene (15 mL) and H₂O (15 mL) was added AA-2 (2.3 g, 10.2 mmol), K₃PO₄ (5.9 g, 28.0 mmol), RuPhos (870 mg, 1.9 mmol) and Pd(OAc)₂ (209 mg, 0.9 mmol) under nitrogen atmosphere. The reaction mixture was stirred at 100° C. overnight. HPLC showed the reaction was completed. Then the mixture was concentrated. The residue was taken up in water, and extracted with EtOAc (×3). The combined organic phase was washed with brine, dried over Na₂SO₄, filtered, and concentrated to give a residue which was purified by silica gel chromatography (PE EtOAc=3:1) to give AA-5 (700 mg). LCMS (ESI, m/z): [M+H]⁺=196.2.

### Step 4 (Synthesis of Compound AA)

To a solution of AA-5 (700 mg, 3.6 mmol) in THE (3.5 mL) was added LiOH·H₂O (226 mg, 5.4 mmol) and H₂O (3.5 mL). The reaction mixture was stirred at room temperature for 1 hour. before it was concentrated. The residue was diluted with water and extracted with EtOAc. The aqueous phase was acidified to pH 2˜3 with aqueous potassium bisulfate solution. The aqueous phase was extracted with DCM:i-PrOH (3:1, ×3). The combined organic phase was dried over Na₂SO₄, filtered and concentrated to give compound AA (270 mg).

The relevant characterization data was as follows: LCMS (ESI, m/z): [M+H]⁺=168.1; ¹HNMR (300 MHz, DMSO-d₆, ppm): δ 12.53 (brs, 1H), 7.48 (s, 1H), 4.00 (s, 3H), 2.44-2.36 (m, 1H), 1.94-1.84 (m, 1H), 1.49-1.41 (m, 1H), 1.33-1.28 (m, 1H).

The intermediates AA1, AA2 below were synthesized followed the similar route of intermediate AA.

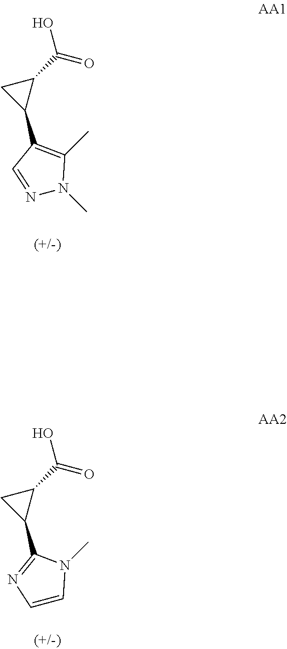

### Intermediate AB

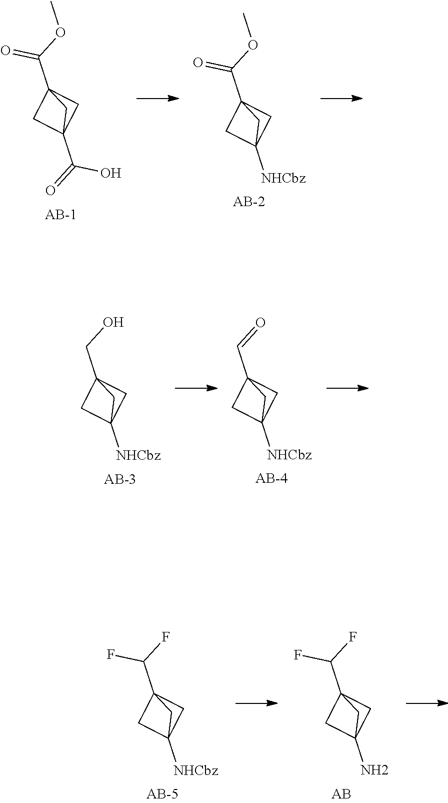 [SMILES: COC(=O)C12CC(C(=O)O)(C1)C2 ; COC(=O)C12CC(NC(=O)OCc3ccccc3)(C1)C2 ; O=C(NC12CC(CO)(C1)C2)OCc1ccccc1 ; O=CC12CC(NC(=O)OCc3ccccc3)(C1)C2 ; O=C(NC12CC(C(F)F)(C1)C2)OCc1ccccc1 ; NC12CC(C(F)F)(C1)C2]

### Step 1 (Synthesis of Compound AB-2)

Under N2, to a solution of AB-1 (5.0 g, 29.4 mmol, 1.0 eq) in toluene (150 mL) was added DPPA (16.2 g, 58.8 mmol, 2.0 eq) and TEA (8.9 g, 88.2 mmol, 3.0 eq), the mixture was stirred at room temperature for 30 minutes, then heated at 120° C. for 3 hours. Cooled to room temperature, benzyl alcohol (9.5 g, 88.2 mmol, 3.0 eq) was added and the mixture was stirred at room temperature for 30 minutes, then heated at 120° C. for 4 hours. HPLC showed the reaction was completed. The reaction was cooled to room temperature, quenched with H₂O and extracted with EtOAc (×3), washed with brine, dried over Na₂SO₄ and concentrated. The residue was purified by column chromatography (PE:EtOAc=10:1) to give AB-2 (4.5 g).

### Step 2 (Synthesis of Compound AB-3)

Under N2, to a solution of AB-1 (4.5 g, 16.3 mmol, 1.0 eq) in THE (90 mL) was added dropwise a solution of LiBH₄ (2M in THF, 24.5 mL, 49.0 mmol, 3.0 eq) at 0° C. The mixture was stirred at room temperature for 3 hours, TLC showed the reaction was completed. The reaction was cooled to 0° C., quenched with NH₄Cl solution and extracted with EtOAc (×3), washed with brine, dried over Na₂SO₄ and concentrated. The residue was purified by column chromatography (PE:EtOAc=3:1) to give AB-3 (4.0 g).

### Step 3 (Synthesis of Compound AB-4)

To the solution of AB-3 (3.5 g, 14.2 mmol, 1.0 eq) in DCM (70 mL) was added Dess-Martin periodinane (12.0 g, 28.3 mmol, 2.0 eq) in portions at 0° C. The reaction was stirred at room temperature for 2 hours. TLC showed the reaction was completed. The mixture was filtered through a pad of celite and washed with DCM (10 mL×3). The filtrate was washed with aqueous saturated NaHCO₃ solution, brine, dried over Na₂SO₄, filtered and concentrated in vacuo. The residue was purified by column chromatography (PE:EtOAc=6:1) to give AB-4 (2.2 g).

### Step 4 (Synthesis of Compound AB-5)

Under N2, to a solution of AB-4 (2.2 g, 9.0 mmol, 1.0 eq) in DCM (40 mL) was added DAST (2.9 g, 17.9 mmol, 2.0 eq) dropwise at −60° C. The reaction was stirred at room temperature for 1 hour. TLC showed the reaction was completed. The mixture was cooled to 0° C., quenched with aqueous saturated NaHCO₃ solution, and extracted with DCM (×3). The organic phase was washed with water and then brine, dried over Na₂SO₄, filtered and concentrated in vacuo to give AB-5 (1.7 g).

### Step 5 (Synthesis of Compound AB)

To a solution of AB-5 (1.6 g, 6.0 mmol, 1.0 eq) in MeOH (16 mL) was added Pd/C (140 mg, 10% wt). The reaction was stirred at room temperature for 2 hours under hydrogen. TLC showed the reaction was completed. The mixture was filtered through a pad of Celite, to the filtrate was added HCl (4 M in EtOAc, 7.5 mL, 30.0 mmol, 5.0 eq). The mixture was stirred at room temperature for 1 hour, concentrated and co-evaporated with toluene for three times to give AB (600 mg).

The relevant characterization data was as follows: ¹HNMR (400 MHz, CDCl₃, ppm): δ 5.79 (t, J=56.4 Hz, 1H), 1.89 (s, 6H).

### Intermediate AC

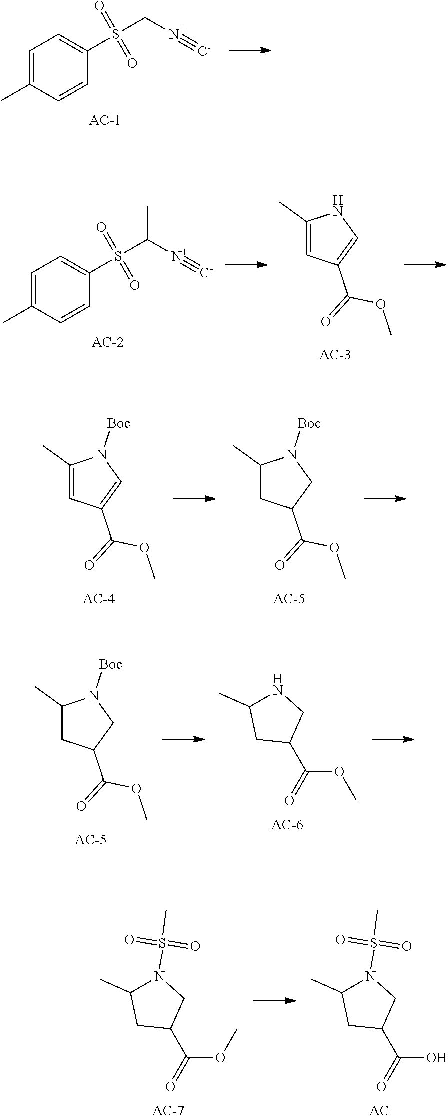

### Step 1 (Synthesis of Compound AC-2

To a solution of AC-1 (30.0 g, 153.7 mmol, 1.0 eq) in DCM (300 mL) was added methyl iodide (45.8 g, 307.4 mmol, 2.0 eq) and benzyltriethylammonium chloride (7.0 g, 30.8 mmol, 0.2 eq). 300 mL of 30% aqueous sodium hydroxide solution was then added at 0° C. The reaction mixture was stirred at 0° C. for 3 hours. TLC showed the reaction was completed. The reaction mixture was diluted with water, extracted with DCM (×2). The combined organic phase was washed with brine, dried over Na₂SO₄, filtered and concentrated to give a residue which was purified by silica gel chromatography (petroleum ether:ethyl acetate=10:1) to give AC-2 (28 g).

### Step 2 (Synthesis of Compound AC-3

Under nitrogen, to a stirred suspension of sodium hydride (4.0 g, 99.4 mmol, 1.6 eq) in diethyl ether (40 mL) was added a solution of methyl acrylate (5.3 g, 62.1 mmol, 1.0 eq) and AC-2 (13.0 g, 62.1 mmol, 1.0 eq) in DMSO (50 mL) and diethyl ether (50 mL) dropwise. The resulted suspension was stirred at room temperature for 2 hours. TLC showed the reaction was completed. Water (100 mL) was added dropwise at 0° C. The aqueous phase was extracted with ethyl acetate (×4), the combined organic layers were washed with water, dried over Na₂SO₄, filtered and concentrated. The residue was purified by silica gel chromatography (petroleum ether:ethyl acetate=5:1) to give AC-3 (5.0 g).

### Step 3 (Synthesis of Compound AC-4

To a solution of AC-3 (3.0 g, 21.6 mmol, 1.0 eq) and (Boc)₂O (5.7 g, 25.9 mmol, 1.2 eq) in DCM (30 mL) were added TEA (6.6 g, 64.8 mmol, 3.0 eq) and DMAP (268 mg, 2.2 mmol, 0.1 eq). The reaction mixture was stirred at room temperature for 12 hours before it was concentrated. The residue was purified by silica gel chromatography (petroleum ether:ethyl acetate=10:1) to give AC-4 (4.6 g).

### Step 4 (Synthesis of Compound AC-5

In a sealed tube, a mixture of AC-4 (2.0 g, 8.4 mmol, 1.0 eq) and Pt/C (2.0 g) in EtOH (20 mL) was stirred at room temperature under 4 MPa of H₂ for 32 hours. The reaction mixture was filtered through a pad of Celite, the filtrate was concentrated under reduced pressure. The residue was purified by silica gel chromatography (petroleum ether:ethyl acetate=5:1) to give AC-5 (1.2 g).

### Step 5 (Synthesis of Compound AC-6

To a solution of AC-5 (730 mg, 3.0 mmol, 1.0 eq) in MeOH (7 mL) was added HCl in Et₂O (15 mL, 30.0 mmol, 10 eq, 2M). Then the mixture was concentrated to give AC-6 (620 mg, crude) as HCl salt, which was used in next step directly without further purification.

### Step 6 (Synthesis of Compound AC-7

To a solution of AC-6 (600 mg, 3.4 mmol, 1.0 eq) in DCM (10 mL) were added Et₃N (1.3 g, 13.2 mmol, 4.0 eq) and MsCl (460 mg, 4.0 mmol, 1.2 eq) at 0° C. The reaction mixture was stirred at room temperature for 2 hours. LCMS showed the reaction was completed. The mixture was quenched with water. The aqueous phase was extracted with DCM (×3). The combined organic phase was washed with brine, dried over Na₂SO₄, filtered and concentrated to give a residue which was purified by silica gel chromatography (petroleum ether:ethyl acetate=2:1) to give AC-7 (370 mg). LCMS (ESI, m/z): [M+H]⁺=222.2.

### Step 7 (Synthesis of Compound AC

To a solution of AC-7 (370 mg, 1.7 mmol, 1.0 eq) in THE (3 mL) was added LiOH·(210 mg, 5.0 mmol, 3.0 eq) and (1 mL). The reaction mixture was stirred at room temperature for 1 hour before it was concentrated. The residue was dissolved in DCM. The aqueous phase was acidified to pH 3 with aqueous citric acid solution. The aqueous phase was extracted with DCM:IPA (3:1, ×3). The combined organic phase was dried over Na₂SO₄, filtered and concentrated to give AC (270 mg), which was used in next step directly without further purification. LCMS (ESI, m/z): [M−H]⁻=206.0.

### Intermediate AD

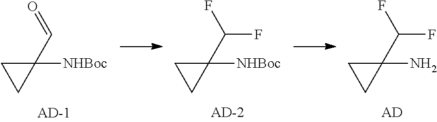 [SMILES: CC(C)(C)OC(=O)NC1(C=O)CC1 ; CC(C)(C)OC(=O)NC1(C(F)F)CC1 ; NC1(C(F)F)CC1]

### Step 1 (Synthesis of Compound AD-2)

To a solution of AD-1 (6.2 g, 33.5 mmol, 1.0 eq) in DCM (120 mL) was added DAST (9.2 g, 56.9 mmol, 1.7 eq) dropwise at 0° C. The reaction mixture was stirred at room temperature for 12 hours. The reaction was quenched by the addition of aqueous saturated NaHCO₃ solution, extracted with DCM (×3). The combined organic layers were washed with brine, dried over Na₂SO₄, and concentrated. The residue was purified by silica gel chromatography (eluted with PE:EtOAc=10:1) to give AD-2 (2.1 g).

### Step 2 (Synthesis of Compound AD

To a solution of AD-2 (2.1 g, 10.1 mmol, 1.0 eq) in dioxane (6 mL) was added HCl/dioxane (4 M, 12.6 mL, 50.5 mmol, 5.0 eq) dropwise at 0° C. The reaction mixture was stirred at room temperature for 2 hours. The reaction mixture was concentrated to give AD (1.5 g, crude) as HCl salt, which was used in next step.

¹HNMR (400 MHz, DMSO-d₆, ppm): δ 9.17 (brs, 3H), 6.02 (t, J=53.6 Hz, 1H), 1.26-1.22 (m, 2H), 1.12-1.07 (m, 2H).

### Intermediate AE

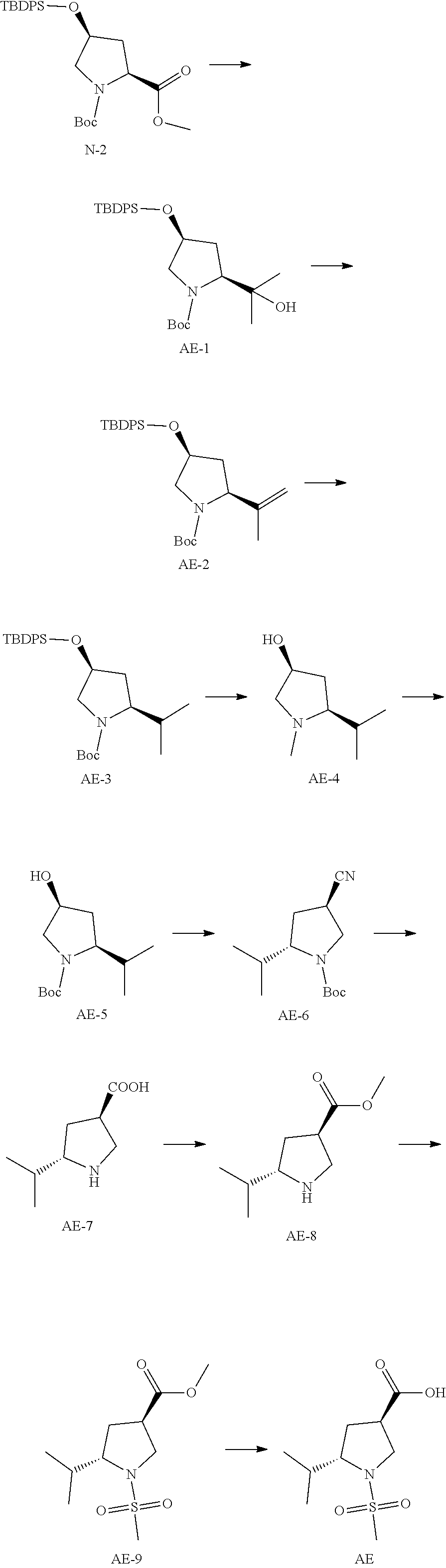

### Step 1 (Synthesis of Compound AE-1)

To a solution of N-2 (15.0 g, 31.0 mmol, 1.0 eq) in THE (105 mL) was added the solution of CH₃MgBr in THE (3.0 mol/L, 42.4 mL, 127.1 mmol, 4.1 eq) at 0° C. under N2 atmosphere. The reaction mixture was stirred at room temperature for 2 hours. TLC showed the reaction was completed. Two batches of parallel reaction were combined and poured into aq. NH₄Cl and extracted with EtOAc (×5). The combined organic layers were washed with brine, dried over Na₂SO₄, filtered and concentrated to give a residue, which was purified by silica gel column chromatography (PE:EtOAc=10:1) to give compound AE-1 (29.8 g).

### Step 2 (Synthesis of Compound AE-2)

To a solution of AE-1 (24.8 g, 51.3 mmol, 1.0 eq) in THE (250 mL) was added dropwise the solution of SOCl₂ (15.3 g, 128.2 mmol, 2.5 eq) in THE (25 mL) at −65° C. under N2 atmosphere. The reaction mixture was stirred at −65° C. for 2 hours. Then to the mixture was added TEA (94.1 g, 922.9 mmol, 18.0 eq) at −65° C. The mixture was stirred at room temperature for 15 hours. before it was partitioned between EtOAc and, and extracted with EtOAc (×3). The combined organic layers were washed with brine, dried over Na₂SO₄, filtered and concentrated to give AE-2 (9.7 g).

### Step 3 (Synthesis of Compound AE-3)

To a solution of AE-2 (5.0 g, 10.7 mmol, 1.0 eq) in EtOH (50 mL) were added Pd/C (1.0 g, 10% w/w) and Pd(OAc)₂ (1.0 g). The mixture was stirred at reflux for 15 hours under H₂ atmosphere before it was filtered through a pad of Celite and the filtrate was concentrated. The residue was purified by silica gel column chromatography (PE:EtOAc=100:1) to give compound AE-3 (4.7 g).

The relevant characterization data was as follows: ¹H NMR (300 MHz, CDCl₃, ppm): δ 7.72-7.60 (m, 4H), 7.48-7.32 (m, 6H), 4.37-2.85 (m, 4H), 2.42-2.18 (m, 1H), 1.98-1.62 (m, 2H), 1.40 (s, 9H), 1.06 (s, 9H), 0.98-0.76 (m, 6H).

### Step 4 (Synthesis of Compound AE-4)

To a solution of AE-3 (4.7 g, 10.1 mmol, 1.0 eq) in THE (50 mL) was added the solution of TBAF in THE (1.0 mol/L, 11.1 mL, 11.1 mmol, 1.1 eq). The mixture was stirred at room temperature for 4 hours before it was diluted with EtOAc, washed with water (×3) and then brine, dried over Na₂SO₄, filtered and concentrated. The crude was purified by silica gel column chromatography (PE:EtOAc=2:1) to give AE-4 (997 mg).

### Step 5 (Synthesis of Compound AE-5)

To a solution of AE-4 (777 mg, 3.4 mmol, 1.0 eq) in DCM (8 mL) were added DIEA (876 mg, 6.8 mmol, 2.0 eq) and MsCl (582 mg, 5.1 mmol, 1.5 eq) at 0° C. under N2 atmosphere. The mixture was stirred at room temperature for 15 hours before it was washed with water and then brine, dried over Na₂SO₄, filtered and concentrated to give AE-5 (1.3 g). LCMS (ESI, m/z): [M+Na]=330.1.

### Step 6 (Synthesis of Compound AE-6)

To a solution of AE-5 (1.3 g, 4.2 mmol, 1.0 eq) in DMSO (15 mL) was added NaCN (1.3 g, 26.2 mmol, 6.2 eq). The mixture was stirred at 80° C. for 15 hours. LCMS showed the reaction was completed. The mixture was cooled and added a saturated aqueous saturated NaHCO₃ solution and extracted with EtOAc (×3). The combined organic layers were washed with brine, dried over Na₂SO₄, filtered and concentrated to give a residue. The residue was purified by silica gel column chromatography (PE:EtOAc=10:1) to give AE-6 (508 mg).

### Step 7 (Synthesis of Compound AE-7)

A solution of AE-6 (508 mg, 2.1 mmol, 1.0 eq) in concentrated HCl (6 mL) was stirred at 80° C. for 2 hours. LCMS showed the reaction was completed. Then the mixture was concentrated to give AE-7 (528 mg, crude), which was used in next step without further purification. LCMS (ESI, m/z): [M+H]⁺=158.2.

### Step 8 (Synthesis of Compound AE-8)

To a solution of AE-7 (528 mg, 2.7 mmol, 1.0 eq) in MeOH (6 mL) was added SOCl₂ (644 mg, 5.4 mmol, 2.0 eq) at 0° C. The mixture was stirred at room temperature for 15 hours before it was concentrated to give AE-8 (503 mg, crude), which was used in next step without further purification. LCMS (ESI, m/z): [M+H]⁺=172.2.

### Step 9 (Synthesis of Compound AE-9)

To a solution of AE-8 (503 mg, 2.4 mmol, 1.0 eq) in DCM (6 mL) were added TEA (732 mg, 7.3 mmol, 3.0 eq) and MsCl (533 mg, 3.6 mmol, 1.5 eq) at 0° C. under N2 atmosphere. The mixture was stirred at room temperature for 15 hours. LCMS showed the reaction was completed. Then the mixture was washed with water and brine, dried over Na₂SO₄, filtered and concentrated to give AE-9 (530 mg, crude), which was used in next step without further purification. LCMS (ESI, m/z): [M+H]⁺=250.1.

### Step 10 (Synthesis of Compound AE)

To a solution of AE-9 (530 mg, 2.1 mmol, 1.0 eq) in MeOH (5 mL) and (5 mL) was added LiOH·(179 mg, 4.3 mmol, 2.0 eq). The mixture was stirred at room temperature for 3 hours. LCMS showed the reaction was completed. The mixture was extracted with DCM (×3) and the aqueous phase was acidified to pH=1 with 3 N HCl solution, and then extracted with DCM (×3). The combined organic layers were washed with brine, dried over Na₂SO₄, filtered and concentrated to give AE-10 (400 mg), which was used in next step without further purification. LCMS (ESI, m/z): [M−H]⁻=234.1.

### Intermediate AF

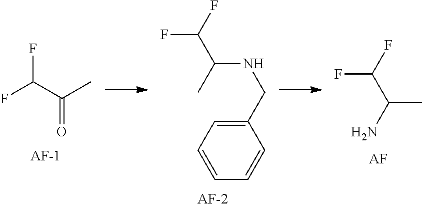

### Step 1 (Synthesis of Compound AF-1)

To a solution of 1,1-difluoropropan-2-one (2 g, 21.3 mmol) in DCM (80 mL) was added BnNH₂ (2.3 g, 21.3 mmol) and NaBH(OAc)₃ (13.5 g, 63.8 mmol). The reaction mixture was stirred at room temperature for 15 hours before it was diluted with aqueous sodium bicarbonate solution. The aqueous phase was extracted with DCM (×3). The combined organic phase was washed with aqueous sodium bicarbonate solution and then brine, dried over Na₂SO₄, filtered and concentrated to give a residue which was purified by silica gel chromatography (PE:EtOAc=50:1) to give compound AF-1 (2.0 g).

### Step 2 (Synthesis of Compound AF

To a solution of AF-1 (1.0 g, 5.4 mmol) in MeOH (20 mL) was added Pd/C (150 mg, 10% wt) at room temperature under hydrogen atmosphere. The reaction mixture was stirred at room temperature for 15 hours before HCl in dioxane (4.8 mL, 27.0 mmol, 4 N in dioxane) was added. The mixture was stirred at room temperature for 1 h and then filtered through a pad of Celite. The filter cake was washed with MeOH. The filtrate was concentrated under reduced pressure to give AF (350 mg).

The relevant characterization data was as follows: ¹H NMR (300 MHz, DMSO-d₆, ppm): δ 8.70 (brs, 3H), 6.26 (dt, J=54.6, 3.0 Hz, 1H), 3.74-3.59 (m, 1H), 1.25 (d, J=6.9 Hz, 3H).

### Intermediate AG

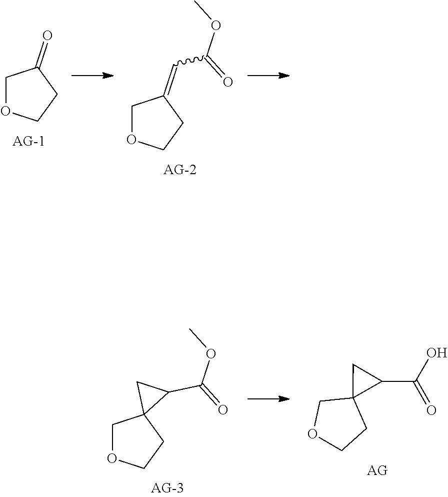

### Step 1 (Synthesis of Compound AG-2)

To a solution of trimethyl phosphonoacetate (126.9 g, 697 mmol, 2.0 eq) in THE (1.8 L) was added NaH (16.7 g, 418 mmol, 1.2 eq, 60% in mineral oil) in portions at 0° C. and stirred for 1.5 hours. Then to the reaction mixture was added a solution of AG-1 (30.0 g, 348 mmol, 1.0 eq) in THE (60 mL), then stirred at room temperature for 12 hours before it was diluted with water and extracted with EtOAc (×3). The combined organic phase was washed with brine, dried over Na₂SO₄ and concentrated. The residue was purified by silica gel chromatography (eluted with PE:EtOAc=10:1) to give AG-2 (37 g).

### Step 2 (Synthesis of Compound AG-3

To a solution of trimethylsulfoxonium iodide (89.2 g, 405 mmol, 1.8 eq) in DMSO (570 mL) was added t-BuOK (45.4 g, 405 mmol, 1.8 eq) at 20° C. and stirred for 1.5 hours. Then to the reaction mixture was added dropwise a solution of AG-2 (32.2 g, 225 mmol, 1.0 eq) in DMSO (250 mL) and stirred at room temperature for 12 hours. The reaction was quenched by the addition of aqueous saturated NH₄Cl solution and extracted with EtOAc (×3). The combined organic phase was washed with brine, dried over Na₂SO₄ and concentrated. The residue was purified by silica gel chromatography (eluted with PE:EtOAc=10:1) to give AG-3 (5.6 g).

### Step 3 (Synthesis of Compound AG)

To a solution of AG-3 (5.6 g, 35.9 mmol, 1.0 eq) in THF/120 (53 mL/53 mL) was added NaOH (2.9 g, 71.8 mmol, 2 eq). Then the reaction mixture was stirred at 40° C. for 1 hour. The reaction was extracted with MTBE. The aqueous phase was acidified to pH=2 with 2N HCl and extracted with EtOAc (×3). The combined organic phase was washed with brine, dried over Na₂SO₄ and concentrated to give AG (4.3 g).

The relevant characterization data was as follows: ¹HNMR (400 MHz, CDCl₃, ppm): δ 8.79 (brs, 1H), 4.14-3.60 (m, 4H), 2.22-1.77 (m, 3H), 1.42-1.20 (m, 2H).

### Intermediate AH

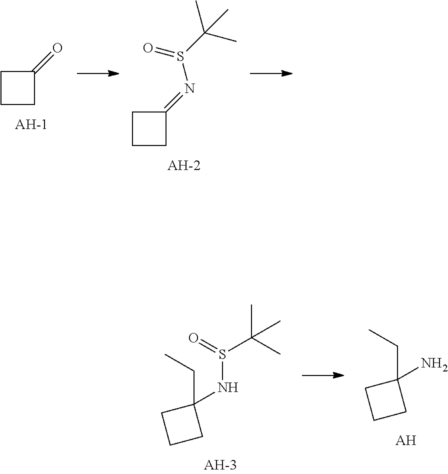

### Step 1 (Synthesis of Compound AH-2)

A mixture of titanium (IV) ethoxide (23.4 g, 102.7 mmol) and AH-1 (4.8 g, 68.5 mmol) in THE (100 mL) was stirred for 10 minutes. 2-Methylpropane-2-sulfinamide (6.89 g, 56.8 mmol) was added and the reaction mixture was stirred at room temperature for 18 hours before it was concentrated. The residue was dissolved in ethyl acetate, washed with saturated aqueous sodium hydrogen carbonate, dried over sodium sulphate, and concentrated to give AH-2 (9 g).

### Step 2 (Synthesis of Compound AH-3)

To the solution of AH-2 (2 g, 11.542 mmol) in DCM (50 mL) was added ethyl magnesium bromide (3M in ethyl ether, 1.9 mL, 5.7 mmol) at −78° C. under N2, then slowly warmed to room temperature and stirred at room temperature for 16 hours. The mixture was quenched with aq. NH₄Cl, extracted with DCM and washed with water. The organic phase was concentrated and purified by chromatography on a silica gel column (petroleum ether to petroleum ether/ethyl acetate=1/2) to give AH-3 (400 mg).

### Step 3 (Synthesis of Compound AH)

To a solution of AH-3 (400 mg, 1.967 mmol) in MeOH (4 mL) was added HCl/ethyl acetate (4 mL, 1 M HCl) at 0° C. and stirred for 3 hours at room temperature. Then the mixture was concentrated and recrystallized with acetonitrile to give AH (100 mg) as HCl salt.

The relevant characterization data was as follows: ¹H NMR (300 MHz, DMSO-d₆, ppm): δ 8.17 (brs, 3H), 2.22-1.71 (m, 8H), 0.91 (t, J=7.2 Hz, 3H).

### Intermediate AI

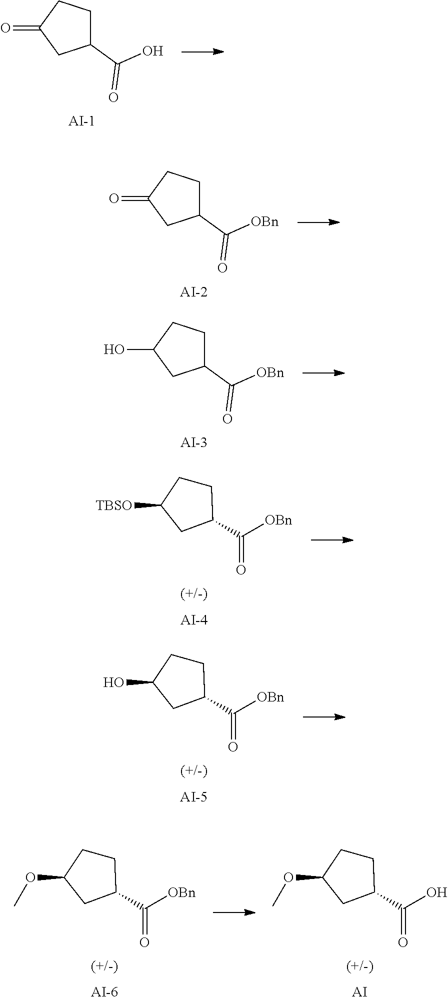

### Step 1 (Synthesis of Compound AI-2)

To a solution of AI-1 (10.0 g, 78.1 mmol, 1.0 eq) in DMF (100 mL) were added BnBr (26.7 g, 156.1 mmol, 2.0 eq) and K₂CO₃ (32.3 g, 234.3 mmol, 3.0 eq). The reaction mixture was stirred at 25° C. for 6 hours before it was diluted with water and extracted with EtOAc (×3). The combined organic phase was washed with water (×4) and then brine, dried over Na₂SO₄, filtered and concentrated to give a residue which was purified by silica gel chromatography (petroleum ether:ethyl acetate=10:1) to give AI-2 (13.0 g).

### Step 2 (Synthesis of Compound AI-3)

To a solution of AI-2 (7.0 g, 32.1 mmol, 1.0 eq) in methanol (70 mL) was added sodium borohydride (1.2 g, 32.1 mmol, 1.0 eq) in portions at 0° C. under the atmosphere of nitrogen and the reaction was stirred at 0° C. for 30 minutes. Acetic acid (2.5 mL) was added to the reaction mixture and stirred for 5 minutes. Methanol was removed under reduced pressure and the residue was taken up in water, extracted with ethyl acetate (×2). Combined organic layer was dried over sodium sulfate and concentrated to give a residue which was purified by silica gel chromatography (petroleum ether:ethyl acetate=5:1) to give AI-3 (4.5 g).

### Step 3 (Synthesis of Compound AI-4)

Under the atmosphere of nitrogen, to a solution of AI-3 (7.5 g, 34.0 mmol, 1.0 eq) in DMF (50 mL) were added imidazole (5.8 g, 85.1 mmol, 2.5 eq) and TBSCl (10.2 g, 68.0 mmol, 2.0 eq). The reaction mixture was stirred at room temperature for 12 hours before it was diluted with water and extracted with EtOAc (×3). The combined organic extracts were washed with water and brine, dried, and concentrated. The residue was purified by silica gel chromatography (eluted with petroleum ether:ethyl acetate=10:1) to give a mixture of cis/rans isomers, then purified by prep-HPLC to give AI-4 (1.5 g,).

### Step 4 (Synthesis of Compound AI-5)

To a solution of AI-4 (1.0 g, 2.9 mmol, 1.0 eq) in THE (10 mL) was added TBAF (5.7 mL, 5.7 mmol, 2.0 eq, 1.0 mol in THF). The reaction mixture was stirred at 25° C. for 6 hours before it was diluted with water and extracted with DCM (×3). The combined organic phase was washed with water (×3) and then brine, dried over Na₂SO₄, filtered and concentrated. The residue was purified by silica gel chromatography (eluted with petroleum ether:ethyl acetate=5:1) to give AI-5 (480 mg).

### Step 5 (Synthesis of Compound AI-6)

To a solution of AI-5 (380 mg, 1.61 mmol, 1.0 eq) in DCM (5 mL) were added 4A molecular sieves (500 mg), silver triflate (1.1 g, 4.2 mmol, 2.6 eq) and MeI (593 mg, 4.2 mmol, 2.6 eq). The reaction mixture was stirred at 25° C. for 12 hours before it was filtered and concentrated. The residue was purified by silica gel chromatography (petroleum ether:ethyl acetate=10:1) to give AI-6 (360 mg).

### Step 6 (Synthesis of Compound AI)

To a solution of AI-6 (350 mg, 1.4 mmol) in EtOAc (5 mL) was added Pd/C (70 mg, 10% wt) and the mixture was stirred at 25° C. overnight under the hydrogen. The reaction mixture was filtered and washed with DCM. The filtrate was concentrated in vacuo to give a residue of AI (180 mg), which was used in next step directly without further purification.

The relevant characterization data was as follows: ¹HNMR (400 MHz, CDCl₃, ppm): δ 3.92-3.88 (m, 1H), 3.27 (s, 3H), 3.02-2.94 (m, 1H), 2.09-1.72 (m, 6H).

### Intermediate AJ

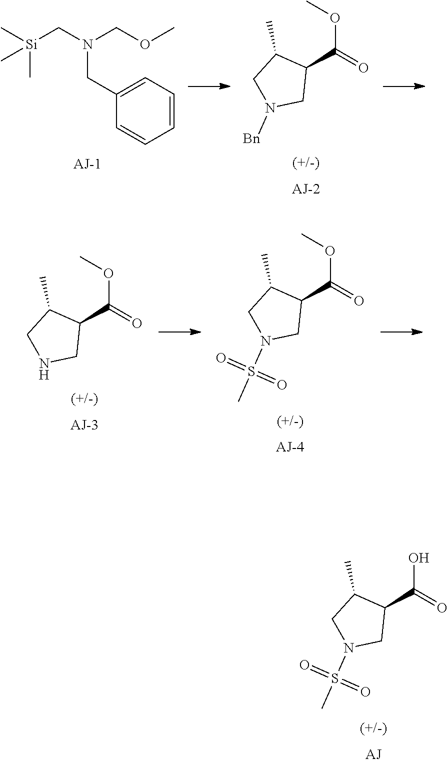

### Step 1 (Synthesis of Compound AJ-2)

To a solution of methyl crotonate (6.6 g, 66.3 mmol, 1.0 eq) in EtOAc (150 mL) and TFA (0.15 mL) was added AJ-1 (15.0 g, 63.2 mmol, 2.0 eq) dropwise. The mixture was then stirred at room temperature overnight before it was diluted with NaHCO₃ solution, stirred for 30 minutes, and extracted with EtOAc (×2). The organic phase was washed with NaHCO₃ solution (×2), dried over Na₂SO₄ and concentrated. The residue was purified on silica gel column chromatography (PE:EA=20:1 to 5:1) to give AJ-2 (8.5 g). LCMS (ES, m/z): [M+H]⁺=234.1.

### Step 2 (Synthesis of Compound AJ-3)

To a solution of AJ-2 (500 mg, 2.7 mmol, 1.0 eq) in EtOH (60 mL) were added Pd(OH)₂ (600 mg) and Pd/C (600 mg) and stirred under 2.4 MPa of hydrogen at 50° C. overnight. HPLC showed the reaction was completed. The mixture was cooled to room temperature and filtered. The filtrate was concentrated and the residue was purified on silica gel column chromatography (PE:EtOAc=5:1 to EA) to give AJ-3 (2.1 g). LCMS (ES, m/z): [M+H]⁺=144.2.

### Step 3 (Synthesis of Compound AJ-4)

Under the atmosphere of nitrogen, to a solution of AJ-3 (2.0 g, 13.9 mmol, 1.0 eq) and Et₃N (3,5 g, 34.7 mnol, 2,5 eq) in DCM (20 mL) was added MsCl (2.4 g, 20.8 mmol, 0.5 eq) dropwise at 0° C. The mixture was stirred for 3 hours before it was diluted with NaHCO₃ solution, extracted with DCM (×3). The combined organic layer was washed with water (×2) and thien brine (×2), dried over Na₂SO₄ and concentrated in vacuo. The residue was purified on silica gel column chromatography (PE:EtOAc=10:1 to 2:1) to give AJ-4 (1.4 g).

### Step 4 (Synthesis of Compound AJ)

To the solution of AJ-4 (1.1 g, 5.0 mmol) in THE (15 mL) was added LiOH·(313 mg, 7.5 mmol) in water (5 mL) and stirred at room temperature overnight. Solvent was removed under reduced pressure, the residue was adjusted to pH=3, extracted with DCM and i-PrOH (3:1, ×3). The combined organic phase was washed with brine, dried over Na₂SO₄, concentrated. The residue was purified on silica gel column chromatography (PE:EtOAc=10:1 to 1:1) to give AJ-4 (800 mg).

The relevant characterization data was as follows: ¹HNMR (400 MHz, DMSO-d₆, ppm): δ 12.62 (brs, 1H), 3.56-3.45 (m, 2H), 3.38-3.35 (m, 1H), 2.90 (s, 3H), 2.87-2.80 (m, 1H), 2.71-2.63 (m, 1H), 2.44-2.33 (m, 1H), 1.07 (d, J=6.4 Hz, 3H).

### Intermediate AK

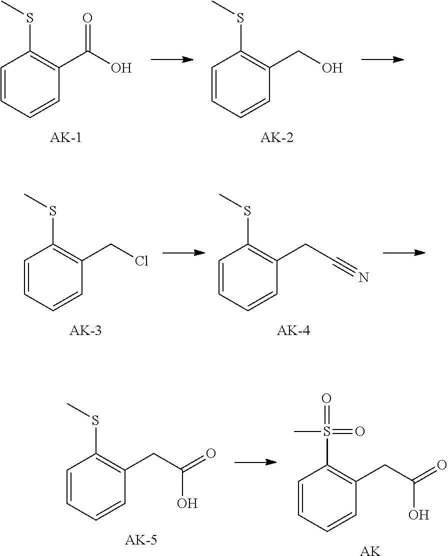

### Step 1 (Synthesis of Compound AK-2)

Under N2, to a solution of AK-2 (4.0 g, 23.8 mmol, 1.0 eq) in THE (50 mL) was added dropwise a solution of BH₃ (59.5 mL, 59.5 mmol, 2.5 eq, 1M in THF) at 0° C. and stirred at room temperature for 2 hours. Water (20 mL) was added dropwise to the mixture at 0° C. and potassium carbonate was added to saturate the solution. the organic solvent was removed and the aqueous phase was extracted with DCM (×3). The combined organic phase was washed with brine (×3), dried over Na₂SO₄, filtered and concentrated. The residue was purified by silica gel chromatography (petroleum ether:ethyl acetate=5:1) to give AK-2 (3.5 g).

### Step 2 (Synthesis of Compound AK-3)

Under N2, to the solution of AK-2 (2.0 g, 13.0 mmol, 1.0 eq) in DCM (10 mL) were added TEA (3.9 g, 39.0 mmol, 3.0 eq) and MsCl (3.0 g, 26.0 mmol, 2.0 eq) at 0° C. The reaction mixture was stirred at room temperature for 7 hours before it was cooled to 0° C. and water (5 mL) was added dropwise. The aqueous phase was extracted with DCM (×3). The combined organic phase was washed with brine (×3), dried over Na₂SO₄, filtered and concentrated to give the residue which was purified by silica gel chromatography (petroleum ether:ethyl acetate=10:1) to give AK-3 (1.0 g).

### Step 3 (Synthesis of Compound AK-4)

Under N₂, to a solution of AK-3 (1.0 g, 5.8 mmol, 1.0 eq) in acetonitrile (10 mL) was added trimethylsilanecarbonitrile (2.8 g, 29.0 mmol, 5.0 eq) and the solution of TBAF in THE (11.6 ml, 11.6 mmol, 2.0 eq, 1M in THF). The reaction mixture was stirred at 40° C. for 16 hours Before water was dropwise added at room temperature. The aqueous phase was extracted with EtOAc (×3). The combined organic phase was washed with brine (×3), dried over Na₂SO₄, filtered and concentrated to give a residue which was purified by silica gel chromatography (petroleum ether:ethyl acetate=10:1) to give AK-4 (850 mg).

The relevant characterization data was as follows: ¹HNMR (300 MHz, METHANOL-d₄, ppm): δ 7.44-7.30 (m, 3H), 7.25-7.17 (m, 1H), 3.91 (s, 2H), 2.51 (s, 3H).

### Step 4 (Synthesis of Compound AK-5)

Under N2, the solution of AK-4 (850 mg, 5.2 mmol, 1.0 eq) in H₂SO₄ (5 mL), HOAc (4 mL) and H₂O (5 mL) was heated at 110° C. for 5 hours. HPLC showed the reaction was completed. Concentrated and the residue was diluted with DCM, washed with brine, dried over Na₂SO₄, filtered and concentrated to give AK-5 (860 mg,). LCMS (ESI, m/z): [M−H]⁻=180.9.

### Step 5 (Synthesis of Compound AK)

Under N2, to a solution of AK-5 (860 mg, 4.7 mmol, 1.0 eq) in DCM (10 mL) was added 85% m-CPBA (2.4 g, 11.8 mmol, 2.5 eq) at 0° C. The reaction mixture was stirred at room temperature for 16 hours before it was filtered and dried over Na₂SO₄, concentrated to give the residue which was purified by silica gel chromatography (petroleum ether:ethyl acetate=1:1) to give AK (800 mg). LCMS (ESI, m/z): [M+H]⁺=215.0.

### Intermediate AL

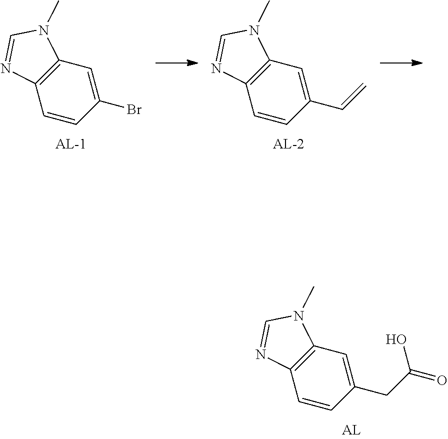

### Step 1 (Synthesis of Compound AL-2)

Under N2, to a solution of AL-1 (500 mg, 2.369 mmol) in dioxane (10 mL) were added potassium vinyltrifluoroborate (634.65 mg, 4.738 mmol) and K₂CO₃ (982.3 mg, 7.107 mmol), then Pd(dppf)Cl₂ (173.3 mg, 0.237 mmol) and H₂O (2 mL). The reaction mixture was stirred for 14 hours at 80° C. before it was cooled to room temperature and concentrated under vacuo. The residue was purified by column chromatography (PE/EA=10/1-1/10) to get AL-2 (298 mg). LCMS (ESI, m/z): [M+H]⁺=159.2.

### Step 2 (Synthesis of Compound AL)

To a solution of AL-2 (345 mg, 2.181 mmol) in 1,2-dimethoxyethane (8 mL) and H₂O (2 mL) were added oxone (2681.3 mg, 4.362 mmol) and 12 (55.4 mg, 0.218 mmol), and the reaction mixture was stirred at room temperature for 4 hours before it was dried over Na₂SO₄, filtered, and concentrated under reduced pressure. The residue was purified by prep-HPLC (0.5% TFA in water/MeCN=10/1˜5/1) to give AL (350 mg). LCMS (ESI, m/z): [M+H]⁺=191.2.

### Intermediate AM

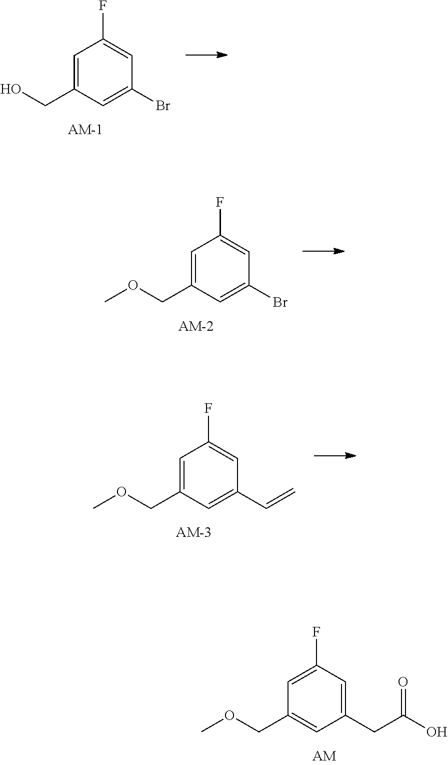

### Step 1 (Synthesis of Compound AM-2)

To a solution of AM-1 (1.0 g, 4.877 mmol) in THE (20 mL) was added NaH (0.2 g, 5.365 mmol) in portions and then CH₃I (0.9 g, 6.341 mmol) was added. The mixture was stirred at 25° C. for 2 hours. The mixture was poured into ice water slowly and extracted by EtOAc (×3). The combined organic layers were dried over Na₂SO₄, filtered, and concentrated under vacuo. The residue was purified by silica gel column chromatography eluted with 0˜20% ethyl acetate in petroleum ether to give AM-2 (800 mg).

### Step 2 (Synthesis of Compound AM-3)

To a solution of AM-2 (800 mg, 3.652 mmol) in dioxane (20 mL)/H₂O (5 mL) were added potassium vinyltrifluoroborate (978.41 mg, 7.304 mmol), K₂CO₃ (1514.3 mg, 10.956 mmol) and Pd(dppf)Cl₂ (267.2 mg, 0.365 mmol). The mixture was stirred at 80° C. for 16 hours. The mixture was quenched with H₂O and extracted by EtOAc (×3). The combined organic layers were dried over Na₂SO₄, filtered, and concentrated under vacuo. The residue was purified by silica gel column chromatography eluted with 0˜30% ethyl acetate in petroleum ether to give AM-3 (520 mg).

### Step 3 (Synthesis of Compound AM)

To a solution of AM-3 (400 mg, 2.41 mmol) in 1,2-dimethoxyethane (20 mL) and H₂O (4 mL) were added 12 (61.1 mg, 0.241 mmol) and oxone (2959.3 mg, 4.81 mmol). The mixture was stirred at 25° C. for 16 hours. The mixture was filtered and concentrated under vacuo. The residue was purified by silica gel column chromatography eluted with 0˜80% ethyl acetate in petroleum ether to give AM (105 mg).

The relevant characterization data was as follows: ¹H NMR (400 MHz, CDCl₃, ppm): δ 6.96 (s, 1H), 6.92-6.83 (m, 2H), 4.37 (s, 2H), 3.57 (s, 2H), 3.33 (s, 3H).

### Intermediate AN

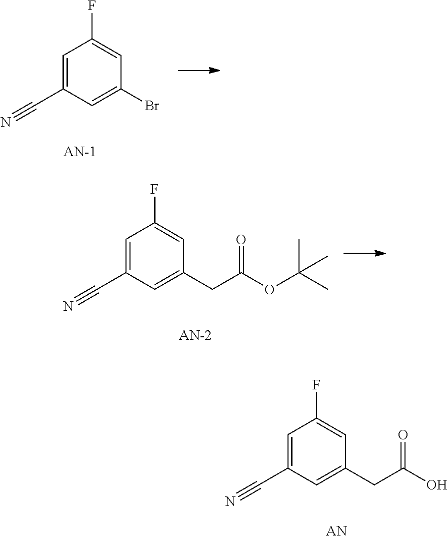

### Step 1 (Synthesis of Compound AN-2)

To a suspension of activated Zn powder (3450 mg, 53.1 mmol) in THE (17 mL) was added chlorotrimethylsilane (0.45 mL, 3.54 mmol) at 25° C. under N2 and the mixture was stirred at 25° C. for 1 hour. Then tert-butyl 2-bromoacetate (3450 mg, 17.7 mmol) was added and stirred at 50° C. under N2 for 1.5 hours. The mixture was cooled, the supernatant was decanted and the residue was used in next step without further purification.

To a solution of AN-1 (400 mg, 2.0 mmol), Pd₂(dba)₃ (183.1 mg, 0.20 mmol) and X-PHOS (190.7 mg, 0.40 mmol) in THF (18 mL) was added the crude product made in above step (6.00 mL, 6.0 mmol) at 25° C. The mixture was stirred at 65° C. under N2 for 3 hours before it was concentrated. The residue was extracted with EtOAc, washed with H₂O, and concentrated. The residue was purified by silica gel chromatography (EtOAc:PE=1:9) to give AN-2 (525 mg). LCMS (ESI, m/z): [M+H]⁺=236.2.

### Step 2 (Synthesis of Compound AN)

To a solution of AN-2 (525 mg, 2.23 mmol) in DCM (8 mL) was added TFA (2 mL, 2.23 mmol) at 25° C. The mixture was stirred at 25° C. for 1 hour before it was concentrated. The residue was purified by silica gel chromatography (MeOH:DCM=1:9) to give AN (293 mg).

The relevant characterization data was as follows: LCMS (ESI, m/z): [M+H]⁺=180.0. ¹H NMR (400 MHz, DMSO-d₆, ppm): δ 7.77-7.74 (m, 1H), 7.64 (s, 1H), 7.57-7.54 (m, 1H), 3.73 (s, 2H).

### Intermediate AO

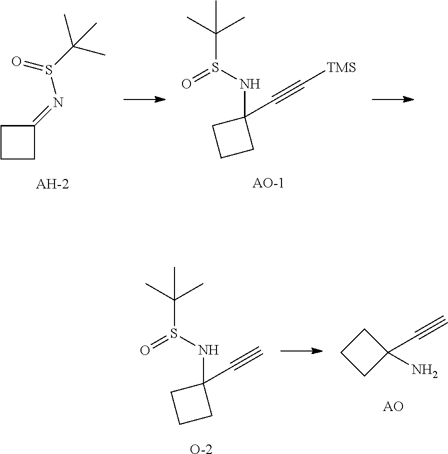

### Step 1 (Synthesis of Compound AO-1)

To a solution of ethynyltrimethylsilane (3.40 g, 34.626 mmol) in THE (50 mL) was added butyllithium solution (1.6 M in ethyl ether, 18.75 mL, 30.009 mmol) at −78° C. under N2, then stirred at −78° C. for 1 hour. AH-2 (2 g, 11.542 mmol) in THE (10 mL) was added dropwise at −78° C., then stirred at −78° C. for 1 hour. The mixture was quenched with H₂O, extracted with EtOAc and washed with water. The organic phase was concentrated and purified by chromatography on a silica gel column (petroleum ether to petroleum ether/ethyl acetate=1/1) to give AO-1 (1 g).

### Step 2 (Synthesis of Compound AO-2)

To a solution of AO-1 (950 mg, 3.499 mmol) in THE (30 mL) was added TBAF (1M in THF, 7 mL, 7 mmol), then stirred at room temperature for 2 hours. Then the reaction mixture was diluted with EtOAc, the organic layer was then washed with H₂O, dried over sodium sulfate and concentrated. The residue was purified by column chromatography on silica gel (petroleum ether to petroleum ether/ethyl acetate=1/1) to give AO-2 (600 mg).

### Step 3 (Synthesis of Compound AO-3)

To a solution of AO-2 (600 mg, 3.010 mmol) in MeOH (4 mL) was added 1 M HCl/ethyl acetate (6 mL) at 0° C. and stirred for 2 hours at room temperature. Then concentrated and the residue was recrystallized in acetonitrile to give AO (350 mg) as HCl salt.

The relevant characterization data was as follows: ¹HNMR (400 MHz, DMSO-d₆, ppm): δ 8.99 (brs, 3H), 3.88 (s, 1H), 2.52-2.41 (m, 2H), 2.35-2.30 (m, 2H), 2.05-1.92 (m, 2H).

### Intermediate AP

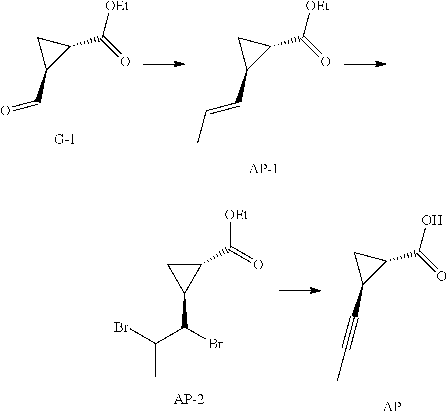

### Step 1 (Synthesis of Compound AP-1)

To a solution of EtPPh₃Br (10.5 g, 28.2 mmol, 2.0 eq) in THE (20 mL) was added dropwise KHMDS (1.0 M in THF, 29.6 mL, 29.6 mmol, 2.1 eq) at −5° C. under N2. The reaction mixture was stirred at −5° C. for 20 minutes and room temperature for 1 hour. Then a solution of G-1 (2.0 g, 14.1 mmol, 1.0 eq) in THE (5 mL) was added dropwise at −5° C. The reaction mixture was stirred at −5° C. for 1 hour and then at room temperature for 12 hours. To the reaction was added aqueous saturated NH₄Cl solution, extracted with EtOAc (×2). The combined organic phase was washed with brine, dried over Na₂SO₄, filtered and concentrated. The residue was purified by column chromatography (PE:EtOAc=50:1) to give AP-1 (605 mg).

### Step 2 (Synthesis of Compound AP-2)

To a solution of AP-1 (605 mg, 3.9 mmol, 1.0 eq) in CHCl₃ (7.8 mL) was added dropwise Br₂ (752 mg, 4.7 mmol, 1.2 eq) at 0° C. under N2. The reaction mixture was stirred at 0° C. for 10 minutes and then stirred at room temperature for 1 hour. TLC showed the reaction was completed. To the mixture was added aqueous saturated Na₂S₂O₃ solution, extracted with DCM (×2). The combined organic phase was washed with brine, dried over Na₂SO₄, filtered and concentrated to give AP-2 (1.1 g, crude), which was used directly in next step without further purification.

### Step 3 (Synthesis of Compound AP)

To a solution of AP-2 (1.1 g, 2.5 mmol, 1.0 eq) in THE (22 mL) was added t-BuOK (981 mg, 8.75 mmol, 2.5 eq). The reaction mixture was heated to reflux for 2 hours before it was cooled to room temperature, diluted with water, and extracted with EtOAc. The aqueous phase was acidified with HCl solution (2N) to pH=2 and extracted with dichloromethane:methanol (10:1, ×3). The combined organic phase was washed with brine, dried over Na₂SO₄, and concentrated to give AP (120 mg).

The relevant characterization data was as follows: ¹HNMR (300 MHz, CDCl₃, ppm): δ 1.91-1.78 (m, 1H), 1.73 (s, 3H), 1.40-1.36 (m, 1H), 1.20-1.15 (m, 1H).

### Intermediate AQ

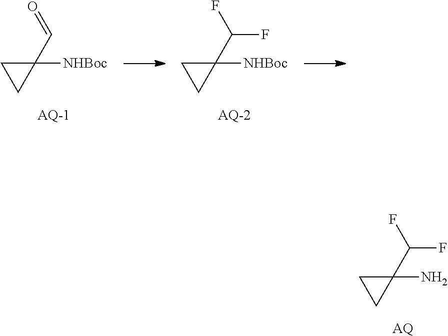

### Step 1 (Synthesis of Compound AQ-2)

To a solution of AQ-1 (6.2 g, 33.5 mmol, 1.0 eq) in DCM (120 mL) was added DAST (9.2 g, 56.9 mmol, 1.7 eq) dropwise at 0° C. The reaction mixture was stirred at room temperature for 12 hours. The reaction was quenched by the addition of aqueous saturated NaHCO₃ solution, extracted with DCM (×3). The combined organic layers were washed with brine, dried over Na₂SO₄ and concentrated to give a residue. The residue was purified by silica gel chromatography (PE:EtOAc=10:1) to give AQ-2 (2.1 g).

### Step 2 (Synthesis of Compound AQ

To a solution of AQ-2 (2.1 g, 10.1 mmol, 1.0 eq) in dioxane (6 mL) was added HCl/dioxane (4 M, 12.6 mL, 50.5 mmol, 5.0 eq) dropwise at 0° C. The reaction mixture was stirred at room temperature for 2 hours before it was concentrated to give AQ-3 (1.5 g, crude) as HCl salt, which was used directly in next step.

The relevant characterization data was as follows: ¹HNMR (400 MHz, DMSO-d₆, ppm): 69.17 (brs, 3H), 6.02 (t, J=53.6 Hz, 1H), 1.26-1.22 (m, 2H), 1.12-1.07 (m, 2H).

### PREPARATION OF EXAMPLES

### Example 1

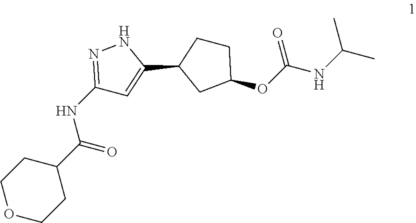 [SMILES: CC(C)NC(=O)O[C@@H]1CC[C@H](c2cc(NC(=O)C3CCOCC3)n[nH]2)C1]

### Synthetic Route for Compound 1 as Follow:

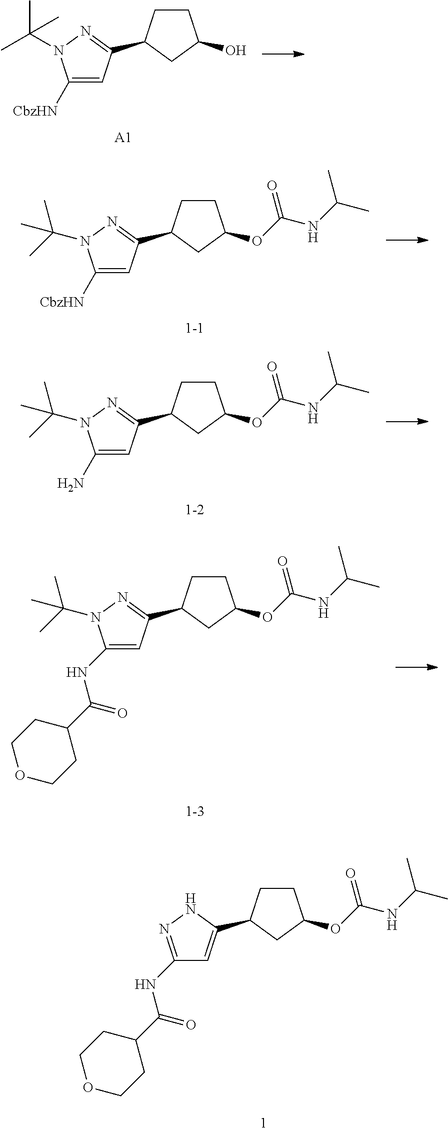

To a solution of A1 (2.1 g, 5.9 mmol, 1.0 eq) and 4-nitrophenyl chloroformate (2.4 g, 11.7 mmol, 2.0 eq) in DCM (20 mL) were added pyridine (1.4 g, 17.6 mmol, 3.0 eq) and DMAP (72 mg, 0.6 mmol, 0.1 eq). The reaction mixture was stirred at room temperature for 3 hours, and then isopropylamine (2.1 g, 35.2 mmol, 6.0 eq) was added. The resulted mixture was stirred at room temperature overnight before it was diluted with EtOAc, washed with aqueous NaOH solution (×3) and saturated aqueous NaHCO₃ solution, dried over Na₂SO₄, filtered and concentrated. The residue was purified by silica gel chromatography (petroleum ether:ethyl acetate=10:1-3:1) to give 1-1 (2.4 g). LCMS (ESI, m/z): [M+H]⁺=443.2.

### Step 2 (Synthesis of Compound 1-2)

To a solution of 1-1 (2.4 g, 5.4 mmol, 1.0 eq) in EtOH (25 mL) was added Pd/C (240 mg, 10 wt %), the reaction mixture was stirred under hydrogen atmosphere at room temperature for 2 hours. HPLC showed the reaction was completed. Then the mixture was filtered through a pad of Celite and concentrated to give 1-2 (1.2 g). LCMS (ESI, m/z): [M+H]⁺=309.2.

### Step 3 (Synthesis of Compound 1-3)

To a solution of 1-2 (120 mg, 0.39 mmol, 1.0 eq) and tetrahydro-2H-pyran-4-carboxylic acid (56 mg, 0.43 mmol, 1.1 eq) in acetonitrile (10 mL) was added DIEA (101 mg, 0.78 mmol, 2.0 eq) and CMPI (129 mg, 0.51 mmol, 1.3 eq) under N2. The reaction mixture was stirred at reflux for 24 hours before it was cooled and diluted with water and extracted with EtOAc (×2). The combined organic layers were washed with brine, dried over Na₂SO₄ and concentrated in vacuo. The crude product was purified by silica gel chromatography (petroleum ether/EtOAc=1:1) to give compound 1-3 (70 mg). LCMS (ESI, m/z): [M+H]⁺=421.3.

### Step 4 (Synthesis of Compound 1)

A solution of 1-3 (70 mg, 0.17 mmol, 1.0 eq) in formic acid (5 mL) was heated at reflux overnight. HPLC showed the reaction was completed, the reaction mixture was concentrated and the crude product was diluted with water and extracted with DCM (×3). The organic extracts were combined, washed with brine, dried over Na₂SO₄ and concentrated in vacuo. The crude product was purified by Prep-HPLC (acetonitrile with 0.1% FA in water, 20% to 36%) to afford compound 1 (22.0 mg).

LCMS (ESI, m/z): [M+H]⁺=365.1; ¹HNMR (400 MHz, METHANOL-d₄, ppm): δ 6.34 (s, 1H), 5.10-5.08 (m, 1H), 4.01-3.98 (m, 2H), 3.71-3.66 (m, 1H), 3.51-3.45 (m, 2H), 3.20-3.13 (m, 1H), 2.68-2.61 (m, 1H), 2.54-2.46 (m, 1H), 2.10-2.08 (m, 1H), 1.95-1.73 (m, 8H), 1.12-1.11 (m, 6H).

### Example 2

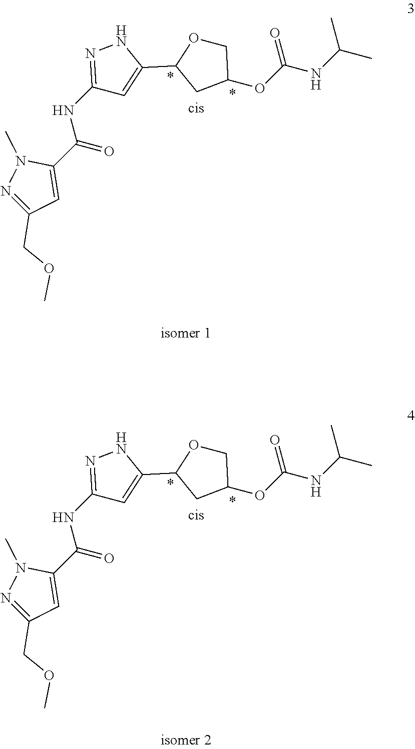 [SMILES: COCc1cc(C(=O)Nc2cc(C3CC(OC(=O)NC(C)C)CO3)[nH]n2)n(C)n1 ; COCc1cc(C(=O)Nc2cc(C3CC(OC(=O)NC(C)C)CO3)[nH]n2)n(C)n1]

### Synthetic Route for Compounds 3 & 4 as Follows:

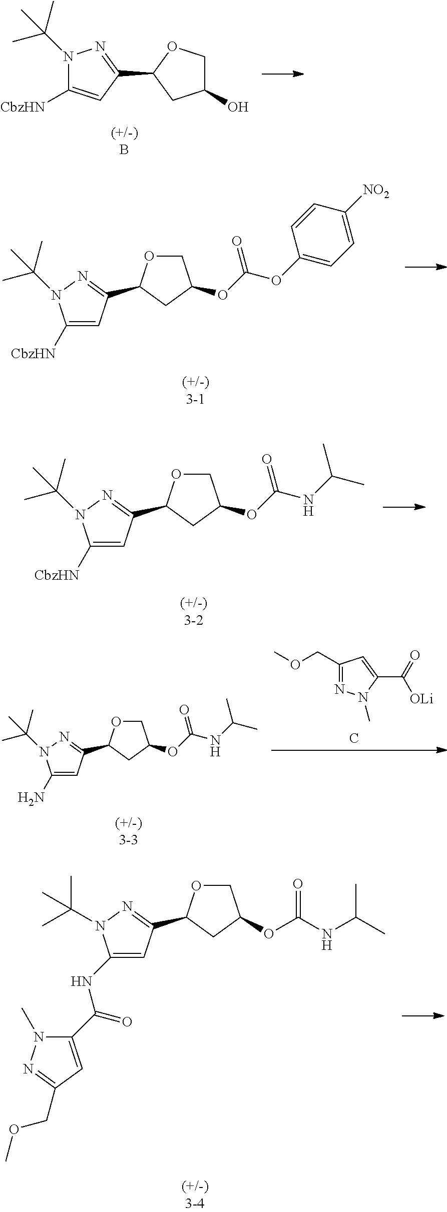

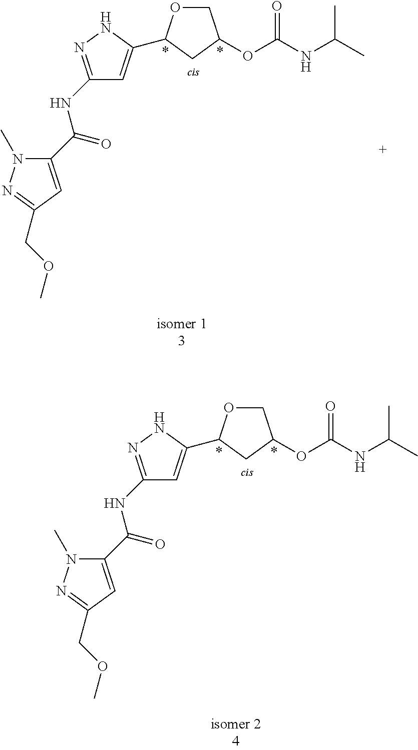

### Step 1 (Synthesis of Compound 3-1)

To a solution of B (800 mg, 2.23 mmol) and 4-nitrophenyl chloroformate (672.9 mg, 3.34 mmol) in dichloromethane (20 mL) were added pyridine (528.2 mg, 6.68 mmol) and 4-dimethylaminopyridine (108.8 mg, 0.89 mmol). The reaction was stirred at room temperature overnight. Another batch of 4-nitrophenyl chloroformate (336 mg, 1.67 mmol) and pyridine (264 mg, 3.34 mol) was added and the reaction was stirred at room temperature for 6 hours. The reaction mixture was concentrated and the residue was purified by silica gel column chromatography eluted with ethyl acetate in petroleum ether from 0% to 50% to give the title product 3-1 (935 mg). LCMS (ESI, m/z): [M+H]⁺=525.2

### Step 2 (Synthesis of Compound 3-2)

To a solution of 3-1 (880 mg, 1.678 mmol) in 2-methyltetrahydrofuran (10 mL) were added N,N-diisopropylethylamine (650.5 mg, 5.033 mmol) and propan-2-amine (148.75 mg, 2.517 mmol) at 0° C. The mixture was warmed to room temperature and stirred for 48 hours. The reaction mixture was concentrated. The residue was diluted with ethyl acetate, washed with 1N sodium hydroxide (×3) and saturated aq. sodium chloride. The organic layer was dried over sodium sulfate, filtered and concentrated to afford the title compound 3-2 (766 mg).

### Step 3 (Synthesis of Compound 3-3)

To a solution of 3-2 (736 mg, 1.66 mmol) in tetrahydrofuran (7 mL) and ethyl acetate (7 mL) was added Pd/C 10% (350 mg, 3.29 mmol) at room temperature. The reaction was degassed and purged with hydrogen (3 times). Then it was stirred at room temperature for 3 hours. This reaction mixture was filtered, concentrated to afford the title compound 3-3 (544 mg, crude). LCMS (ESI, m/z): [M+H]⁺=311.2

### Step 3 (Synthesis of Compound 3-4)

To a solution of 3-3 (396.59 mg, 1.28 mmol) in acetonitrile (35 mL) was added C (150 mg, 0.85 mmol) and N, N-diisopropylethylamine (330.3 mg, 2.56 mmol), then CMPI (435.2 mg, 1.70 mmol) was added. The reaction mixture was stirred at room temperature for 3 hours before it was cooled to room temperature and most of the solvent was removed. The residue was purified by C18 column (eluted with acetonitrile in 0.05% trifluoroacetic acid in water from 5% to 40%) to give compound 3-4 (142 mg). LCMS (ESI, m/z): [M+H]⁺=463.4

### Step 5 (Synthesis of Compounds 3 & 4)

To a flask containing 3-4 (130 mg, 0.211 mmol) was added formic acid (5 mL). The mixture was stirred at 100° C. for 3 hours. The solvent was removed and the residue was purified by prep-HPLC (eluted with acetonitrile in 0.05% trifluoroacetic acid in water from 5% to 25%), then separated via SFC [Waters SFC 150, DAICELCHIRALCEL® OZ, 250*25 mm 10 m, MeOH (0.1% 7.0 mol/L Ammonia in MeOH)/Supercritical CO₂], then further purified by prep-HPLC (eluted with acetonitrile in 0.05% trifluoroacetic acid in water from 5% to 30%) to give compound 3 (isomer 1, 20.4 mg) and 4 (isomer 2, 20.6 mg).

The relevant characterization data for compound 3 was as follows: LCMS (ESI, m/z): [M+H]⁺=406.9; ¹HNMR (400 MHz, DMSO-d₆, ppm): δ 10.76 (s, 1H), 7.09-7.05 (m, 2H), 6.50 (s, 1H), 5.15-5.14 (m, 1H), 4.83 (t, J=7.6 Hz, 1H), 4.33 (s, 2H), 4.04 (s, 3H), 3.85-3.83 (m, 2H), 3.58-3.55 (m, 2H), 3.25 (s, 3H), 2.69-2.64 (m, 1H), 1.95-1.91 (m, 1H), 1.02 (d, J=6.4 Hz, 6H). Chiral purity 100.0% ee, retention time 4.145 min. Chiral SFC analysis was performed on a Waters UPCC (CA-060), DAICEL CHIRALPAK® OZ, 100*3.0 mm 3 μm column heated to 35° C., eluted with a mobile phase of CO₂ and gradient of 5-40% MeOH (0.1% DEA), flowing at 1.5 mL/min.

The relevant characterization data for compound 4 was as follows: LCMS (ESI, m/z): [M+H]⁺=406.9; ¹HNMR (400 MHz, DMSO-d₆, ppm): δ 10.76 (s, 1H), 7.09-7.05 (m, 2H), 6.50 (s, 1H), 5.15-5.14 (m, 1H), 4.83 (t, J=7.6 Hz, 1H), 4.33 (s, 2H), 4.04 (s, 3H), 3.85-3.83 (m, 2H), 3.58-3.55 (m, 2H), 3.25 (s, 3H), 2.71-2.64 (m, 1H), 1.95-1.91 (m, 1H), 1.02 (d, J=6.4 Hz, 6H). Chiral purity 97.7% ee, retention time 4.567 min. Chiral SFC analysis was performed on a Waters UPCC (CA-060), DAICELCHIR-ALPAK® OZ, 100*3.0 mm 3 μm column heated to 35° C., eluted with a mobile phase of CO₂ and gradient of 5-40% MeOH (0.1% DEA), flowing at 1.5 mL/min.

### Example 3

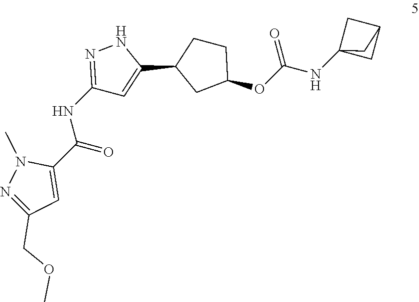 [SMILES: COCc1cc(C(=O)Nc2cc([C@H]3CC[C@@H](OC(=O)NC45CC(C4)C5)C3)[nH]n2)n(C)n1]

### Synthetic Route for Compound 5 as Follows:

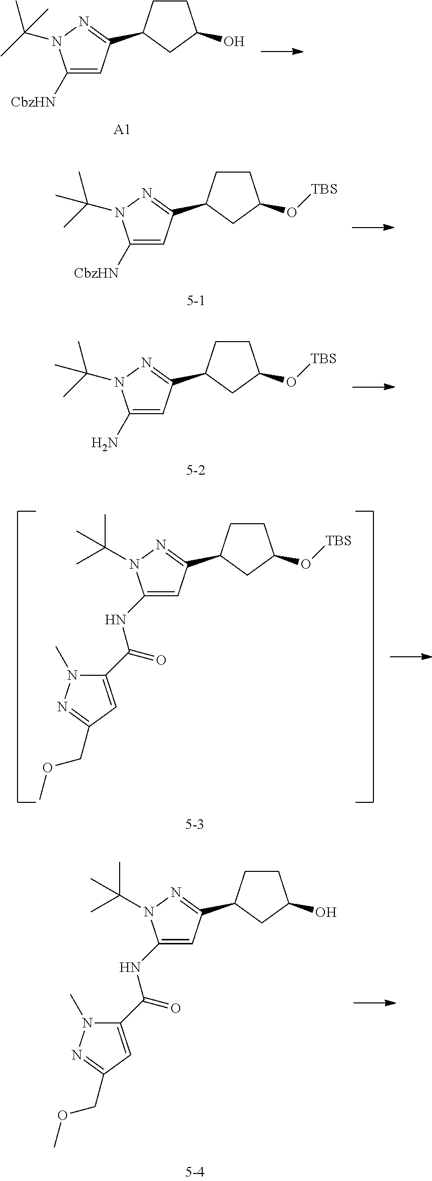

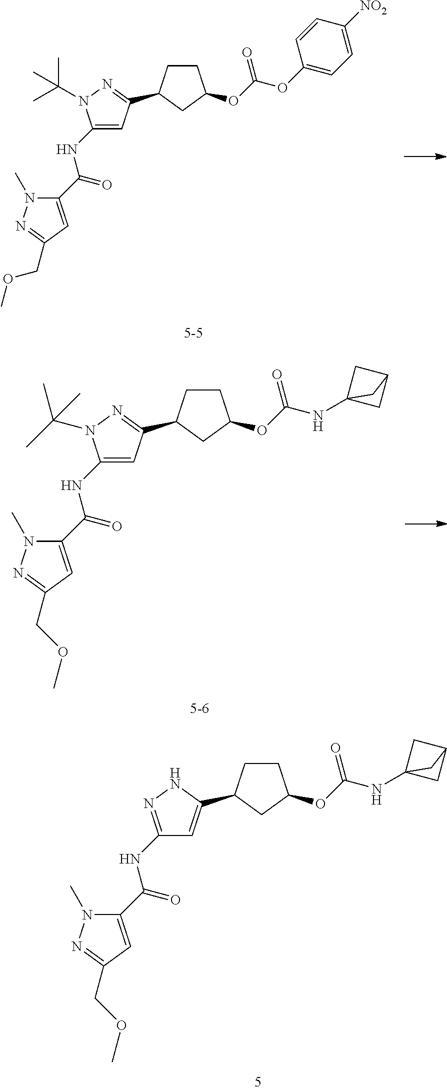 [SMILES: COCc1cc(C(=O)Nc2cc([C@H]3CC[C@@H](OC(=O)Oc4ccc([N+](=O)[O-])cc4)C3)nn2C(C)(C)C)n(C)n1 ; COCc1cc(C(=O)Nc2cc([C@H]3CC[C@@H](OC(=O)NC45CC(C4)C5)C3)nn2C(C)(C)C)n(C)n1 ; COCc1cc(C(=O)Nc2cc([C@H]3CC[C@@H](OC(=O)NC45CC(C4)C5)C3)[nH]n2)n(C)n1]

### Step 1 (Synthesis of Compound 5-1)

To a solution of A1 (2.0 g, 5.59 mmol, 1.0 eq) in DMF (20 mL) was added imidazole (570.5 mg, 8.38 mmol, 1.5 eq) and TBSCl (1.09 g, 7.26 mmol, 1.3 eq). The mixture was stirred at room temperature for 2 hours. HPLC showed the reaction was completed. The reaction mixture was diluted with EtOAc and the organic layer was washed with brine, dried over Na₂SO₄, filtered and concentrated to give the residue which was purified by silica gel chromatography (PE:EtOAc=10:1) to give 5-1 (2.5 g). LCMS (ESI, m/z): [M+H]⁺=472.3.

### Step 2 (Synthesis of Compound 5-2)

To a solution of 5-1 (1.2 g, 2.54 mmol, 1.0 eq) in EtOAc (6 mL) and THE (6 mL) was added Pd/C (200 mg, 10% wt). The mixture was stirred at room temperature under H₂ for 5 hours. HPLC showed the reaction was completed. The mixture was filtered through a pad of Celite and washed with EtOAc. The filtrate was concentrated to give the residue which was purified by silica gel chromatography (PE:EtOAc=10:1) to give 5-2 (800 mg). LCMS (ESI, m/z): [M+H]⁺=338.3.

### Step 3 (Synthesis of Compound 5-4)

The solution of 5-2 (200 mg, 0.59 mmol, 1.0 eq) in acetonitrile (2 mL) were added C1 (151.3 mg, 0.89 mmol, 1.5 eq), CMPI (301.4 mg, 1.18 mmol, 2.0 eq) and DIEA (228.7 mg, 1.77 mmol, 3.0 eq). The reaction was stirred at 60° C. for 1 hour before it was cooled to room temperature and stirred at room temperature overnight. The mixture was diluted with EtOAc, washed with brine (×3), dried over Na₂SO₄, filtered and concentrated to give the residue which was purified by silica gel chromatography (PE:EtOAc=5:1) to give 5-4 (150 mg). LCMS (ESI, m/z): [M+H]⁺=376.3

### Step 4 (Synthesis of Compound 5-5)

To a solution of 5-4 (300.0 mg, 0.80 mmol, 1.0 eq) in DCM (5 mL) were added 4-nitrophenyl chloroformate (320.5 mg, 1.59 mmol, 2.0 eq), pyridine (189.8 mg, 2.40 mmol, 3.0 eq) and DMAP (9.7 mg, 0.08 mmol, 0.1 eq) at 0° C. The reaction mixture was stirred at room temperature for 5 hours before it was diluted with water, extracted with DCM (×3). The organic phase was washed with brine, dried over Na₂SO₄, filtered and concentrated to give the residue which was purified by silica gel chromatography (PE EtOAc=3:1) to give 5-5 (230 mg). LCMS (ESI, m/z): [M+H]⁺=541.2

### Step 5 (Synthesis of Compound 5-6)

To a solution of 5-5 (70.0 mg, 0.13 mmol, 1.0 eq) in THE (2 mL) were added bicyclo[1.1.1]pentan-1-amine hydrochloride (46.6 mg, 0.39 mmol, 3.0 eq) and DIEA (84.0 mg, 0.65 mmol, 5.0 eq). The reaction mixture stirred at room temperature for 2 hours before water was added and the mixture was extracted with EtOAc (×3). The combined organic phase was dried over Na₂SO₄, filtered and concentrated to give the residue which was purified by prep-TLC (DCM:MeOH=10:1) to give 5-6 (37 mg). LCMS (ESI, m/z): [M+H]⁺=485.3.

### Step 6 (Synthesis of Compound 5)

A solution of 5-6 (38 mg, 0.08 mmol, 1.0 eq) in formic acid (1 mL) was stirred at 70° C. for 12 hours. HPLC showed the reaction was completed. DCM (10 mL) was added and the organic phase was washed with aqueous saturated sodium bicarbonate solution (×3), dried over Na₂SO₄, filtered and concentrated to give the residue which was purified by prep-TLC (DCM:MeOH=10:1) to give 5 (13.0 mg).

The relevant characterization data was as follows: LCMS (ESI, m/z): [M+H]⁺=429.1; ¹H NMR (400 MHz, METHANOL-d₄, ppm): δ 6.92 (s, 1H), 6.43 (s, 1H), 5.13-5.02 (m, 1H), 4.43 (s, 2H), 4.12 (s, 3H), 3.37 (s, 3H), 3.20-3.10 (m, 1H), 2.55-2.45 (m, 1H), 2.35 (m, 1H), 2.15-2.05 (m, 1H), 1.99 (s, 6H), 1.90-1.70 (m, 4H).

### Example 4

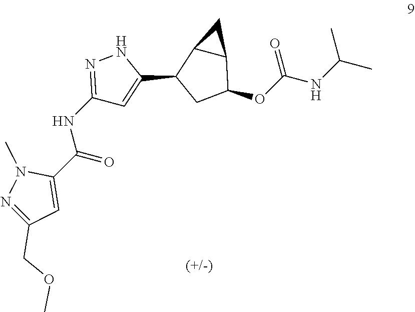 [SMILES: COCc1cc(C(=O)Nc2cc([C@@H]3C[C@H](OC(=O)NC(C)C)[C@@H]4C[C@@H]43)[nH]n2)n(C)n1]

### Synthetic Route for Compound 9 as Follows:

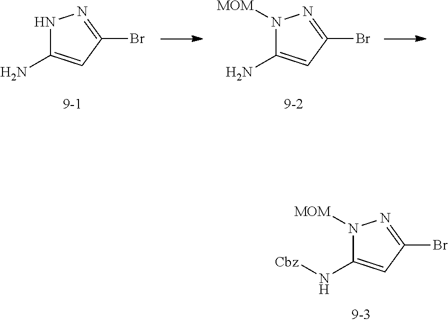

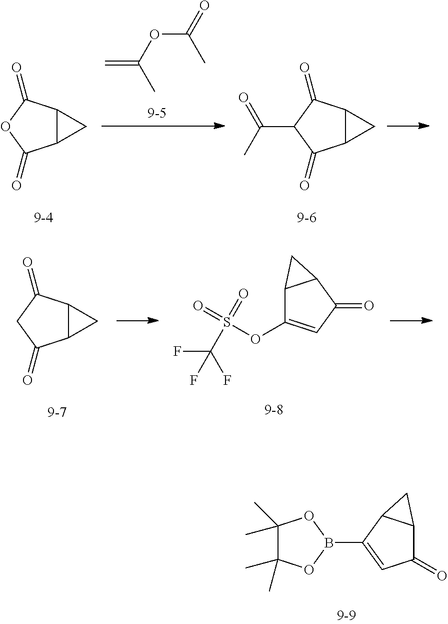

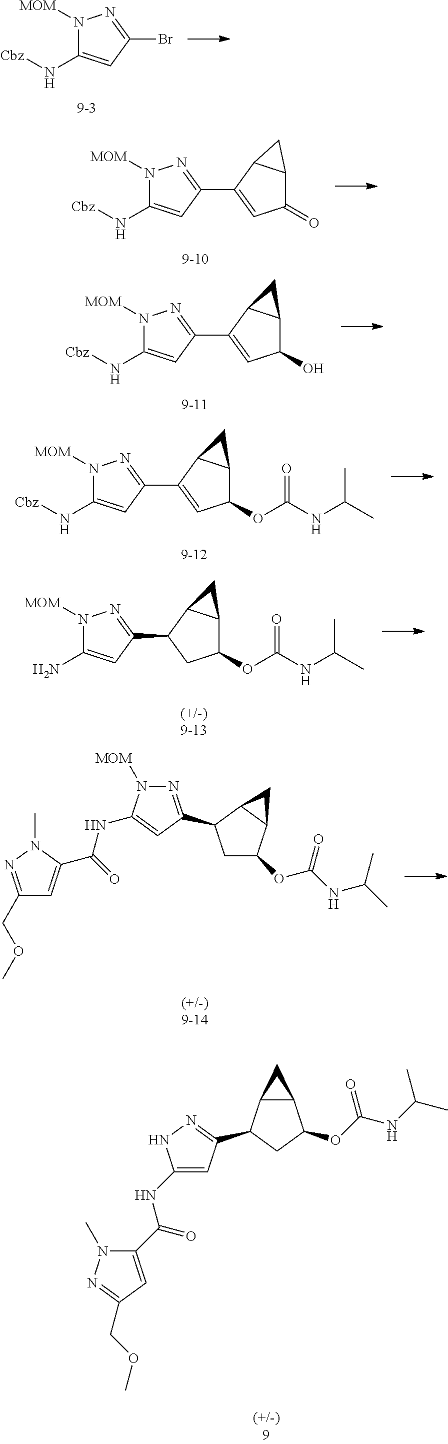

### Step 1 (Synthesis of Compound 9-2)

To a solution of 9-1 (5 g, 30.9 mmol) in tetrahydrofuran (50 mL) was added sodium hydride (1.9 g, 46.3 mmol) at 0° C. in portions. The reaction mixture was stirred for 20 minutes. Bromo(methoxy)methane (3.86 g, 30.9 mmol) was added into the above reaction. The reaction was stirred for 12 hours at room temperature before it was quenched with water, extracted with ethyl acetate. The organic layer was dried over Na₂SO₄, filtered and concentrated. The residue was purified through silica gel column (eluted with ethyl acetate in petroleum ether from 0 to 40%) to give 9-2 (2.7 g). LCMS (ESI, m/z): [M+H]⁺=206.1

### Step 2 (Synthesis of Compound 9-3)

To a mixture of 9-2 (2.7 g, 13.1 mmol) and sodium bicarbonate (3.3 g, 39.3 mmol) in tetrahydrofuran (30 mL) was added benzyl chloroformate (2.767 mL, 19.6 mmol). The reaction mixture was stirred at room temperature for 12 hours before it was washed with water and extracted with ethyl acetate. The organic layer was dried over sodium sulphate, filtered and concentrated. The residue was purified by silica gel column (eluted with ethyl acetate in petroleum ether from 0 to 30%) to give 9-3 (2.5 g). LCMS (ESI, m/z): [M+H]⁺=340.1.

### Step 3 (Synthesis of Compound 9-6)

To a vigorously stirred solution of 9-4 (22 g, 196.3 mmol) in 1,2-dichloroethane (300 mL) was added aluminium trichloride (52.9 g, 396.5 mmol) in portions controlled inner temperature below 10° C. Then to the above reaction mixture was added a solution of prop-1-en-2-yl acetate (19.65 g, 196.3 mmol) in 1,2-dichloroethane (30 mL) dropwise while maintaining inner temperature below 20° C. Then the ice bath was removed. The reaction solution was stirred at 30° C. for 13 hours under N2 before it was cooled to room temperature and poured into a mixture of ice and 2N HCl. The resulting mixture was stirred for 30 minutes. The organic layer was separated and the water layer was extracted with ethyl acetate (×3). The organic layer was dried over Na₂SO₄, filtered and concentrated. The residue was purified by silica gel column (eluted with ethyl acetate in petroleum ether from 0 to 35%) to give 9-6 (6.5 g). LCMS (ESI, m/z): [M+H]⁺=153.10

### Step 4 (Synthesis of Compound 9-7)

A suspension of 9-6 (100 mg, 0.657 mmol) in 2N HCl (12 mL) was stirred at 100° C. for 3 hours in a sealed tube. This reaction was repeated 22 times. The combined reaction mixture of was extracted with ethyl acetate (×3) and DCM/MeOH:10/1 (×2). The organic layer was dried over sodium sulphate, filtered and concentrated. The residue was purified by silica gel column (eluted with ethyl acetate in petroleum ether from 0 to 30%) to give 9-7 (2 g).

### Step 5 (Synthesis of Compound 9-8)

To a solution of 9-7 (500 mg, 4.541 mmol) and 2,6-dimethylpyridine (0.793 mL, 6.811 mmol) in dichloromethane (6 mL) was added trifluoromethanesulfonic anhydride (0.904 mL, 5.449 mmol) at −78° C. in portions. The reaction mixture was stirred at the temperature for 3 hours before it was diluted with ethyl acetate and washed with 1N HCl. The organic layer was dried over sodium sulphate, filtered and concentrated in vacuo. The residue was separated through silica gel column (eluted with ethyl acetate in petroleum ether from 0 to 30%) to give 9-8 (450 mg). LCMS (ESI, m/z): [M+H]⁺=243.0.

### Step 6 (Synthesis of Compound 9-9

To a solution of 9-8 (450 mg, 1.86 mmol), 4,4,5,5-tetramethyl-2-(tetramethyl-1,3,2-dioxaborolan-2-yl)-1,3,2-dioxaborolane (943.7 mg, 3.72 mmol) and KOAc (364.7 mg, 3.72 mmol) in 1,4-dioxane (8 mL) was added Pd(dppf)Cl₂ (275.1 mg, 0.38 mmol). The reaction mixture was stirred at 100° C. for 2 hours under N2 before it was cooled and diluted with ethyl acetate and washed with water. The organic layer was dried over sodium sulphate, filtered and concentrated in vacuo. The residue was purified through silica gel column chromatography (eluted with ethyl acetate in petroleum ether from 0 to 25%) to give compound 9-9 (340 mg).

### Step 7 (Synthesis of Compound 9-10)

To a mixture of 9-3 (92.74 mg, 0.273 mmol) and 9-9 (60 mg, 0.273 mmol) and K₂CO₃ (113.0 mg, 0.818 mmol) in 1,4-dioxane (6 mL) and water (0.6 mL) was added Pd(dppf)Cl₂ (39.9 mg, 0.055 mmol). The reaction mixture was stirred at 100° C. under N2 for 12 hours before it was cooled and diluted with EtOAc and washed with water. The organic layer was dried over Na₂SO₄, filtered and concentrated in vacuo. The residue was purified through silica gel column chromatography (eluted with ethyl acetate in petroleum ether from 0 to 60%) to give compound 9-10 (28 mg). LCMS (ESI, m/z): [M+H]⁺=354.2.

### Step 8 (Synthesis of Compound 9-11)

To a solution of 9-10 (400.0 mg, 1.13 mmol) in THE (20 mL) was added lithium triethylborohydride (3.40 mL, 3.40 mmol) slowly at −78° C. The reaction mixture was allowed to warm to −10° C. in 2 hours before it was quenched with water and extracted with ethyl acetate (×3). The organic layer was dried over sodium sulphate, filtered and concentrated in vacuo. The residue was purified by silica gel column chromatography (eluted with ethyl acetate in petroleum from 0 to 60%) to give 9-11 (400 mg). LCMS (ESI, m/z): [M+H]⁺=356.4.

### Step 9 (Synthesis of Compound 9-12)

To a solution of 9-11 (400 mg, 1.13 mmol) in THE (10 mL) was added n-butyllithium (3.377 mL, 3.38 mmol) at −78° C. dropwise. The reaction mixture was stirred at that temperature for 15 minutes. Then 2-isocyanatopropane (239.2 mg, 2.81 mmol) was added into the above reaction mixture. The reaction mixture was stirred for 3 hours at room temperature before it was quenched with water and diluted with THF, dried over anhydrous sodium sulfate, filtered and concentrated. The residue was purified through silica gel column chromatography (eluted with ethyl acetate in petroleum ether from 0 to 60%) to give compound 9-12 (240 mg). LCMS (ESI, m/z): [M+H]⁺=441.3.

### Step 10 (Synthesis of Compound 9-13)

To a solution of 9-12 (220 mg, 0.499 mmol) in THE (10 mL) and ethyl acetate (10 mL) was added Pd/C (150 mg, 0.454 mmol) and the reaction was stirred under H₂ for 1 hour. The mixture was filtered and concentrated. The residue was purified through silica gel column chromatography (eluted with ethyl acetate in petroleum ether from 0 to 60%) to compound 9-13 (130 mg). LCMS (ESI, m/z): [M+H]⁺=309.2.

### Step 11 (Synthesis of Compound 9-14)

To the solution of 9-13 (120 mg, 0.389 mmol) dissolved in acetonitrile (5 mL) was added C1 (132.44 mg, 0.778 mmol) and DIEA (0.322 mL, 1.95 mmol), CMPI (397.7 mg, 1.56 mmol) and the reaction was stirred at 80° C. for 2 hours. The reaction mixture was concentrated in vacuum. The residue was purified by silica gel column (eluted with ethyl acetate in petroleum ether from 0 to 90%), then purified by pre-HPLC (eluted with acetonitrile in 0.5% trifluoroacetic acid in water from 5% to 32%) to give 9-14 (70 mg). LCMS (ESI, m/z): [M+H]⁺=461.4.

### Step 12 (Synthesis of Compound 9)

A mixture 9-14 (30 mg, 0.065 mmol) and p-toluenesulfonic acid (33.7 mg, 0.195 mmol) in 1,4-dioxane (1 mL) was stirred at 85° C. for 16 hours. The reaction mixture was purified by pre-HPLC (eluted with acetonitrile in 0.5% trifluoroacetic acid in water from 5% to 32%) to give 9 (2.4 mg).

The relevant characterization data was as follows: LCMS (ESI, m/z): [M+H]⁺=417.4; ¹H NMR (400 MHz, DMSO-d₆, ppm) δ: 12.29 (br, 1H), 10.75 (s, 1H), 7.12 (s, 1H), 7.04 (d, J=7.4 Hz, 1H), 6.46 (s, 1H), 5.21-5.16 (m, 1H), 4.34 (s, 2H), 4.05 (s, 3H), 3.62-3.56 (m, 1H), 3.55-3.53 (m, 1H), 3.27 (s, 3H), 2.31-2.18 (m, 1H), 1.66-1.63 (m, 2H), 1.31-1.15 (m, 1H), 1.06-1.03 (m, 6H), 0.71-0.69 (m, 1H), 0.51-0.44 (m, 1H).

### Example 5

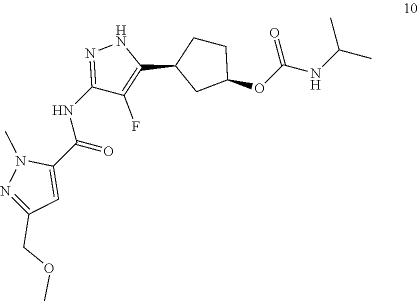 [SMILES: COCc1cc(C(=O)Nc2n[nH]c([C@H]3CC[C@@H](OC(=O)NC(C)C)C3)c2F)n(C)n1]

### Synthetic Route for Compound 9 as Follows:

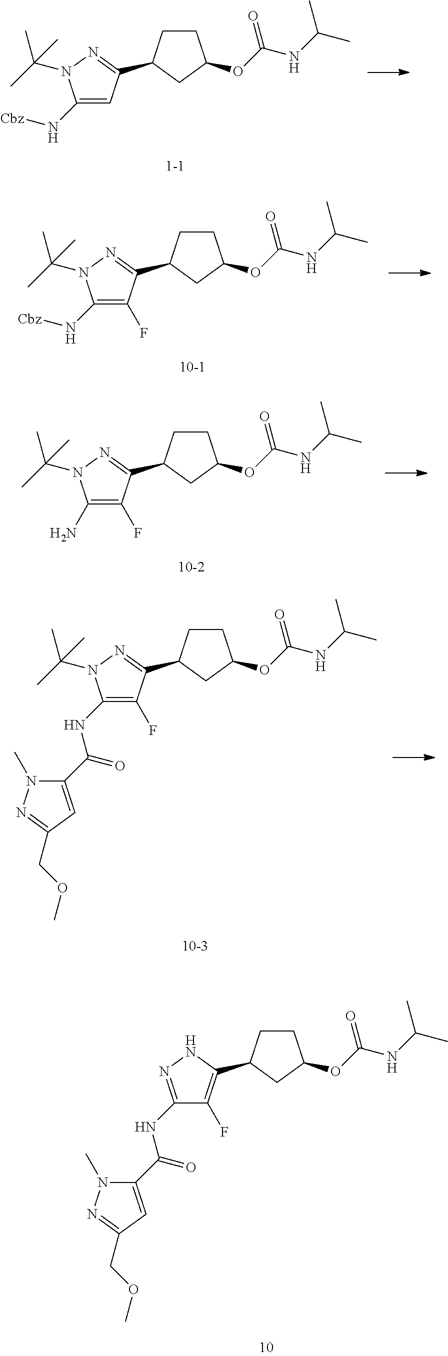

### Step 1 (Synthesis of Compound 10-1)

To a solution of 1-1 (220 mg, 0.50 mmol, 1.0 eq) in DCM (13 mL) and DMF (13 mL) was added selectfluor (352 mg, 0.99 mmol, 2.0 eq) at 0° C. The reaction mixture was stirred at room temperature for 2 hours before it was diluted with EtOAc, then washed with brine, dried over Na₂SO₄, filtered and concentrated. The residue was purified by silica gel chromatography (PE:EtOAc=10:1 to 3:1) to give 10-1 (160 mg). LCMS (ESI, m/z): [M+H]⁺=461.3

### Step 2 (Synthesis of Compound 10-2)

To a solution of 10-1 (160 mg, 0.35 mmol, 1.0 eq) in EtOH (8 mL) was added Pd/C (32 mg, 10 wt %), the reaction mixture was stirred under hydrogen atmosphere at room temperature for 2 hours before it was filtered through a pad of Celite and concentrated to give compound 10-2 (100 mg). LCMS (ESI, m/z): [M+H]⁺=327.3

### Step 3 (Synthesis of Compound 10-3)

To the solution of C (200 mg, 1.2 mmol, 1.0 eq) dissolved in DCM (4 mL) was added SOCl₂ (700 mg, 5.9 mmol, 5.0 eq) and DMF (9 mg, 0.1 mmol, 0.1 eq), the reaction mixture was stirred at 40° C. for 4 hours before it was concentrated to give the cured product which was dissolved in DCM (9 mL). 10-2 (90 mg, 0.27 mmol, 1.0 eq) and Et₃N (84 mg, 0.83 mmol, 3.0 eq) were added to the above solution. The reaction mixture was stirred at room temperature overnight and diluted with DCM, then washed with water and brine, dried over Na₂SO₄, filtered and concentrated to give a residue which was purified by Prep-TLC (PE:EtOAc=1:1) to give 10-3 (140 mg).

### Step 4 (Synthesis of Compound 10)

A solution of 10-3 (140 mg, crude) in formic acid (2 mL) was heated at reflux for 2 hours. HPLC showed the reaction was completed. The resulted mixture was concentrated and water was added and extracted with DCM (×3). The organic phase was washed with brine, dried over Na₂SO₄ and concentrated in vacuo. The crude product was purified by prep-HPLC (acetonitrile with 0.1% FA in water, 30% to 36%) to give 10 (30 mg).

The relevant characterization data was as follows: LCMS (ESI, m/z): [M+H]⁺=423.2. ¹HNMR (400 MHz, METHANOL-d₄, ppm): δ 6.97 (s, 1H), 5.15-5.101 (m, 1H), 4.45 (s, 2H), 4.12 (s, 3H), 3.74-3.66 (m, 1H), 3.38 (s, 3H), 3.24-3.18 (m, 1H), 2.59-2.45 (m, 1H), 2.14-2.10 (m, 1H), 2.04-1.87 (m, 4H), 1.12 (d, J=6.4 Hz, 6H).

### Example 6

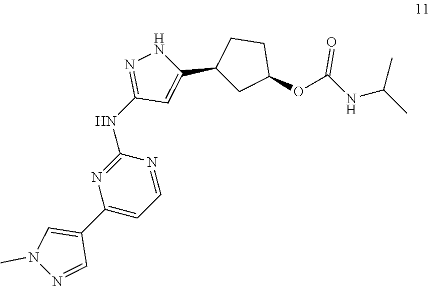 [SMILES: CC(C)NC(=O)O[C@@H]1CC[C@H](c2cc(Nc3nccc(-c4cnn(C)c4)n3)n[nH]2)C1]

### Synthetic Route for Compound 11 as Follows:

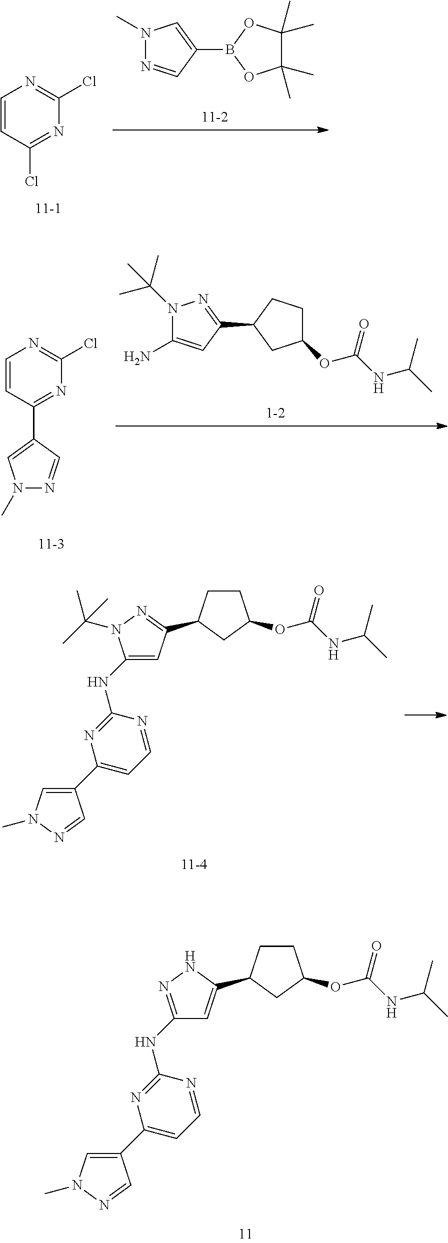

### Step 1 (Synthesis of Compound 11-3)

To a solution of 11-1 (3.5 g, 23.5 mmol, 1.0 eq) in acetonitrile (80 mL) were added 11-2 (5.2 g, 24.8 mmol, 1.05 eq), Pd(dppf)Cl₂·CH₂Cl₂ (575 mg, 0.7 mmol, 0.03 eq), Na₂CO₃ (5.0 g, 47.2 mmol, 2.0 eq) and H₂O (14 mL) at room temperature under nitrogen atmosphere. The reaction mixture was stirred at 110° C. for 6 hours before it was cooled and diluted with water. The aqueous phase was extracted with EtOAc (×3). The combined organic phase was washed with brine, dried over Na₂SO₄, filtered and concentrated to give a residue which was purified by silica gel chromatography (eluted with petroleum ether:ethyl acetate=1:1) to give 11-3 (1.6 g). LCMS (ESI, m/z): [M+H]⁺=195.1.

### Step 2 (Synthesis of Compound 11-4)

To a solution of 1-2 (130 mg, 0.4 mmol, 1.0 eq) in 2-methyltetrahydrofuran (2 mL) were added 11-3 (164 mg, 0.8 mmol, 2.0 eq), Cs₂CO₃ (481 mg, 1.5 mmol, 3.5 eq), H₂O (2 mL), BrettPhos (54 mg, 0.1 mmol, 0.24 eq) and Pd₂(dba)₃ (58 mg, 0.1 mmol, 0.15 eq) in sealed tube at room temperature under nitrogen atmosphere. The reaction mixture was stirred at 100° C. for 3 hours before it was cooled and diluted with water. The aqueous phase was extracted with EtOAc (×3). The combined organic phase was washed with brine, dried over Na₂SO₄, filtered and concentrated. The residue was purified by silica gel chromatography (dichloromethane:methanol=40:1) to give 11-4 (120 mg). LCMS (ESI, m/z): [M+H]⁺=467.3.

### Step 3 (Synthesis of Compound 11)

The solution of 11-4 (120 mg, 0.3 mmol, 1.0 eq) in anhydrous formic acid (5 mL) was stirred at 70° C. for 2 hours. HPLC showed the completion of reaction. Then the mixture was concentrated to give a residue which was purified by prep-HPLC (acetonitrile with 0.1% FA in water, 20% to 34%) to give 11 (56.8 mg).

The relevant characterization data was as follows: LCMS (ESI, m/z): [M+H]⁺=411.2; ¹HNMR (400 MHz, METHANOL-d₄, ppm): δ 8.33 (d, J=5.2 Hz, 1H), 8.27 (s, 1H), 8.09 (s, 1H), 7.03 (d, J=5.2 Hz, 1H), 6.33 (s, 1H), 5.12-5.09 (m, 1H), 3.76 (s, 3H), 3.70-3.67 (m, 1H), 3.20-3.14 (m, 1H), 2.58-2.51 (m, 1H), 2.14-2.13 (m, 1H), 2.10-1.91 (m, 4H), 1.15-1.05 (m, 6H).

### Example 7

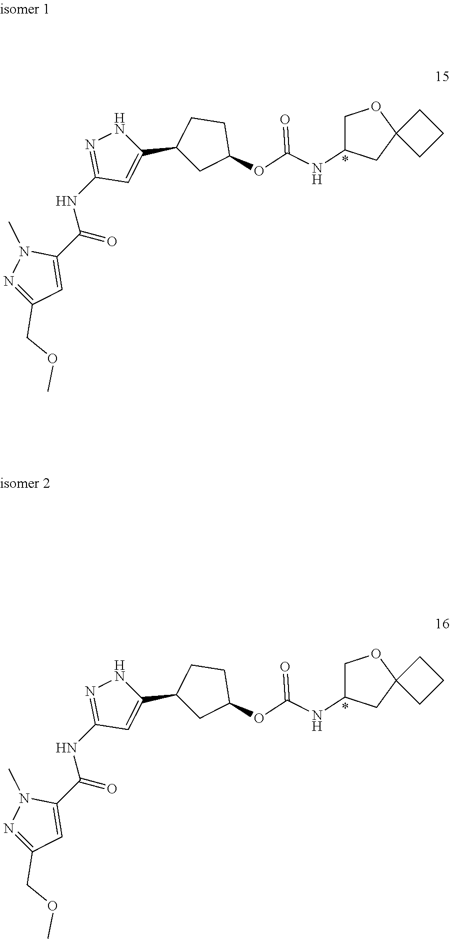 [SMILES: COCc1cc(C(=O)Nc2cc([C@H]3CC[C@@H](OC(=O)NC4COC5(CCC5)C4)C3)[nH]n2)n(C)n1 ; COCc1cc(C(=O)Nc2cc([C@H]3CC[C@@H](OC(=O)NC4COC5(CCC5)C4)C3)[nH]n2)n(C)n1]

### Synthetic Route for Compounds 15 & 16 as Follows:

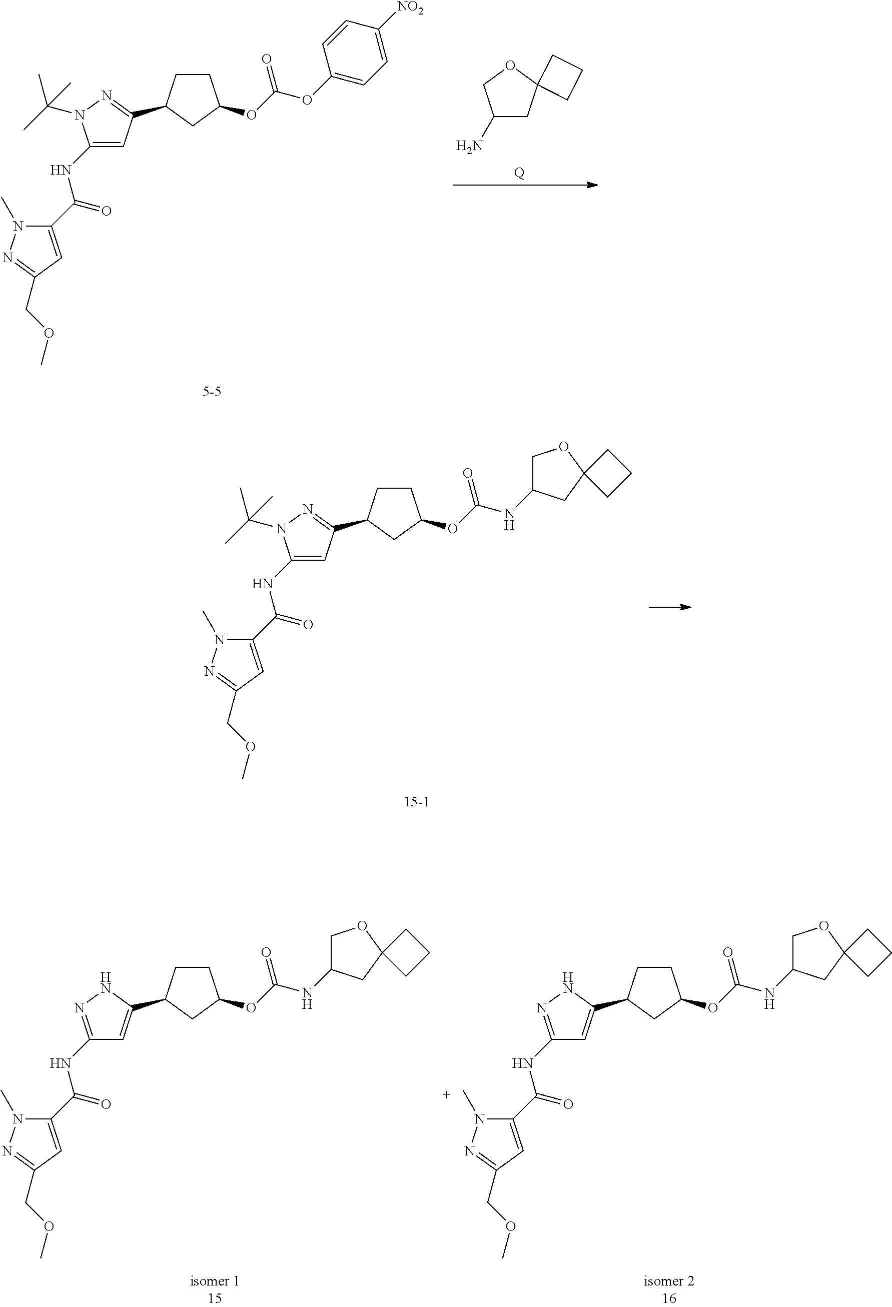

### Step 1 (Synthesis of Compound 15-1)

To a solution of Q (108 mg, 0.85 mmol, 2.0 eq) in THE (10 mL) were added 5-5 (230 mg, 0.43 mmol, 1.0 eq) and DIEA (165 mg, 1.28 mmol, 3.0 eq). The reaction mixture was stirred at room temperature for 12 hours before it was concentrated to give a residue which was purified by prep-TLC (DCM:MeOH=80:1) to give 15-1 (200.0 mg). LCMS (ESI, m/z): [M+H]⁺=529.0.

### Step 2 (Synthesis of Compounds 15 & 16)

The solution of 15-1 (180.0 mg, 0.34 mmol, 1.0 eq) in anhydrous formic acid (5 mL) was stirred at 70° C. for 12 hours. Then the mixture was concentrated to give a residue which was purified by prep-TLC (DCM:MeOH=20:1), then separated via SFC (column: OD-H, IPA (0.1% of DEA)/CO₂=20/80) to give compound 15 (isomer 1, 31.8 mg) and compound 16 (isomer 2, 39.1 mg).

The relevant characterization data for compound 15 was as follows: LCMS (ESI, m/z): [M+H]⁺=473.2. ¹H NMR (400 MHz, DMSO-d₆, ppm): δ 12.22 (brs, 1H), 10.72 (brs, 1H), 7.32 (d, J=6.4 Hz, 1H), 7.11 (s, 1H), 6.42 (s, 1H), 5.04-4.98 (m, 1H), 4.34 (s, 2H), 4.11-3.96 (m, 4H), 3.85-3.79 (m, 1H), 3.45-3.38 (m, 1H), 3.27 (s, 3H), 3.10-3.06 (m, 1H), 2.19-1.41 (m, 14H). Chiral purity 99.11% de, retention time 15.113 min. Chiral SFC analysis was performed on OD-3 column heated to 35° C., eluted with a mobile phase of CO₂ and gradient of 20% IPA (0.1% of DEA), flowing at 2.0 mL/min

The relevant characterization data for compound 16 was as follows: LCMS (ESI, m/z): [M+H]⁺=473.2. ¹H NMR (400 MHz, DMSO-d₆, ppm): δ 12.22 (brs, 1H), 10.72 (brs, 1H), 7.32 (d, J=6.4 Hz, 1H), 7.11 (s, 1H), 6.42 (s, 1H), 5.04-4.98 (m, 1H), 4.34 (s, 2H), 4.11-3.96 (m, 4H), 3.85-3.79 (m, 1H), 3.45-3.38 (m, 1H), 3.27 (s, 3H), 3.10-3.06 (m, 1H), 2.18-1.40 (m, 14H). Chiral purity 92.44% de, retention time 17.902 min. Chiral SFC analysis was performed on OD-3 column heated to 35° C., eluted with a mobile phase of CO₂ and gradient of 20% IPA (0.1% of DEA), flowing at 2.0 mL/min.

### Example 8

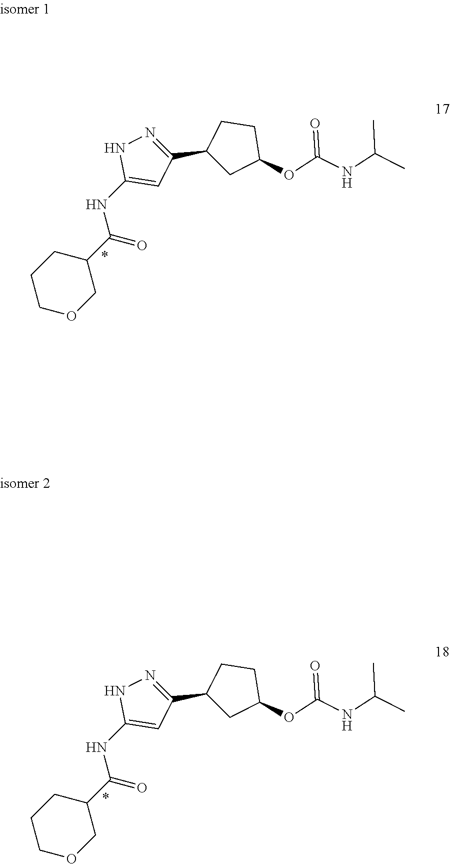 [SMILES: CC(C)NC(=O)O[C@@H]1CC[C@H](c2cc(NC(=O)C3CCCOC3)[nH]n2)C1 ; CC(C)NC(=O)O[C@@H]1CC[C@H](c2cc(NC(=O)C3CCCOC3)[nH]n2)C1]

### Synthetic Route for Compounds 17 & 18 as Follows:

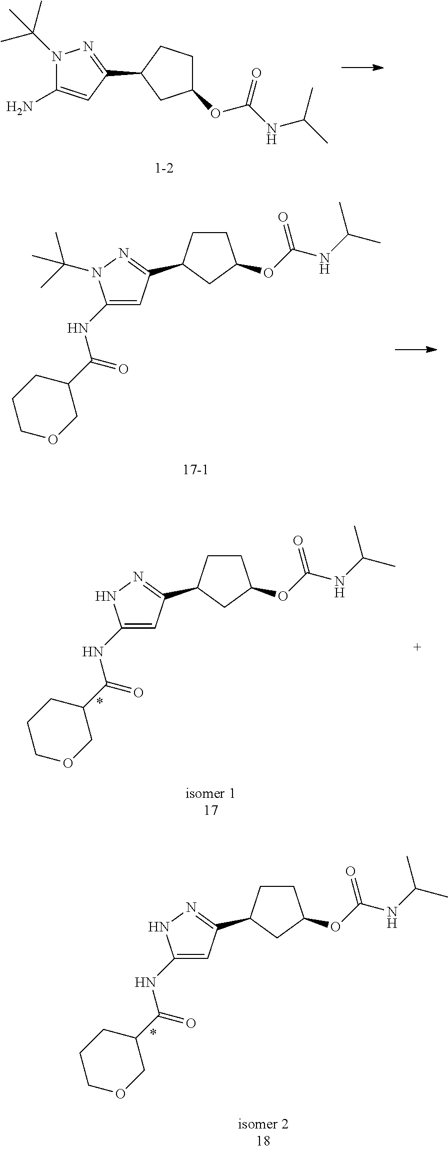 [SMILES: CC(C)NC(=O)O[C@@H]1CC[C@H](c2cc(N)n(C(C)(C)C)n2)C1 ; CC(C)NC(=O)O[C@@H]1CC[C@H](c2cc(NC(=O)C3CCCOC3)n(C(C)(C)C)n2)C1 ; CC(C)NC(=O)O[C@@H]1CC[C@H](c2cc(NC(=O)C3CCCOC3)[nH]n2)C1 ; CC(C)NC(=O)O[C@@H]1CC[C@H](c2cc(NC(=O)C3CCCOC3)[nH]n2)C1]

### Step 1 (Synthesis of Compound 17-1)

To a solution of 1-2 (220.0 mg, 0.71 mmol, 1.0 eq) and tetrahydro-2H-pyran-3-carboxylic acid (102.1 mg, 0.78 mmol, 1.1 eq) in acetonitrile (11 mL) were added DIEA (184.4 mg, 1.43 mmol, 2.0 eq) and CMPI (242.4 mg, 0.95 mmol, 1.3 eq) under N2. The reaction mixture was stirred at 78° C. for 24 hours before it was cooled and diluted with water and extracted with DCM (×2). The organic phase was washed with brine, dried over Na₂SO₄ and concentrated in vacuo and the residue was purified by Pre-TLC (petroleum ether/EtOAc=1:1) to give compound 17-1 (200.0 mg), which was used in next step directly. LCMS (ESI, m/z): [M+H]⁺=421.3

### Step 2 (Synthesis of Compounds 17 & 18)

A solution of 17-1 (200.0 mg, 1.0 eq) in formic acid (20 mL) was heated at reflux overnight. HPLC showed the reaction was completed. The reaction mixture was cooled and concentrated and the residue was diluted with water and extracted with DCM (×3). The organic phase was washed with brine, dried over Na₂SO₄ and concentrated in vacuo. The residue was purified by Pre-TLC (DCM/MeOH=20:1), then separated via chiral SFC (DAICELCHIRALCEL® AS, EtOH (0.1% 7.0 mol/l Ammonia in MeOH)/CO₂=30/70), further purified by Pre-HPLC (acetonitrile with 0.1% FA in water, 20% to 38%) to give compound 17 (isomer 1, 19.0 mg) and 18 (isomer 2, 20.0 mg).

The relevant characterization data for compound 17 was as follows: LCMS (ESI, m/z): [M+H]⁺=365.2. ¹HNMR (400 MHz, DMSO-d₆, ppm): δ 12.02 (brs, 1H), 10.26 (brs, 1H), 6.96-6.90 (m, 1H), 6.29 (s, 1H), 5.05-4.95 (m, 1H), 3.90-3.86 (m, 1H), 3.85-3.75 (m, 1H), 3.60-3.50 (m, 1H), 3.37-3.32 (m, 1H), 3.28-3.20 (m, 1H), 3.10-2.95 (m, 1H), 2.68-2.58 (m, 1H), 2.50-2.38 (m, 1H), 1.99-1.93 (m, 1H), 1.90-1.80 (m, 2H), 1.75-1.40 (m, 6H), 1.02 (d, J=6.4 Hz, 6H). Chiral purity 97.98% de, retention time 9.644 min. Chiral SFC analysis was performed on a Waters UPCC (CA-060), DAICEL CHIRALPAK® OZ, 100*3.0 mm 3 μm column heated to 35° C., eluted with a mobile phase of CO₂ and gradient of 5-40% MeOH (0.1% DEA), flowing at 1.5 mL/min.

The relevant characterization data for compound 18 was as follows: LCMS (ESI, m/z): [M+H]⁺=365.2. ¹HNMR (400 MHz, DMSO-d₆, ppm): δ 12.01 (brs, 1H), 10.26 (brs, 1H), 6.96-6.90 (m, 1H), 6.29 (s, 1H), 5.05-4.95 (m, 1H), 3.90-3.86 (m, 1H), 3.85-3.75 (m, 1H), 3.60-3.50 (m, 1H), 3.37-3.32 (m, 1H), 3.28-3.20 (m, 1H), 3.10-2.95 (m, 1H), 2.68-2.58 (m, 1H), 2.50-2.38 (m, 1H), 1.99-1.93 (m, 1H), 1.90-1.80 (m, 2H), 1.75-1.40 (m, 6H), 1.02 (d, J=6.4 Hz, 6H). Chiral purity 95.00% de, retention time 11.879 min. Chiral SFC analysis was performed on a Waters UPCC (CA-060), DAICEL CHIRALPAK® OZ, 100*3.0 mm 3 μm column heated to 35° C., eluted with a mobile phase of CO₂ and gradient of 5-40% MeOH (0.1% DEA), flowing at 1.5 mL/min.

### Example 9

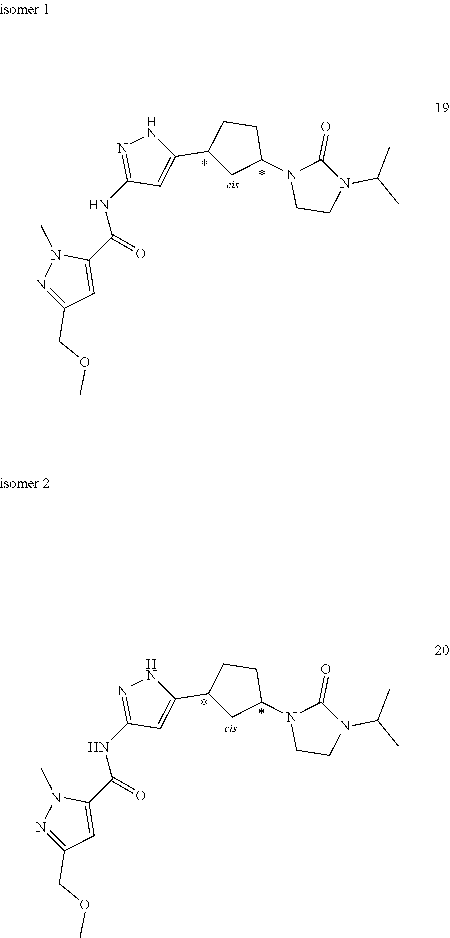 [SMILES: COCc1cc(C(=O)Nc2cc(C3CCC(N4CCN(C(C)C)C4=O)C3)[nH]n2)n(C)n1 ; COCc1cc(C(=O)Nc2cc(C3CCC(N4CCN(C(C)C)C4=O)C3)[nH]n2)n(C)n1]

### Synthetic Route for Compounds 19 & 20 as Follows:

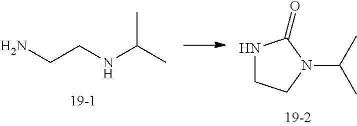

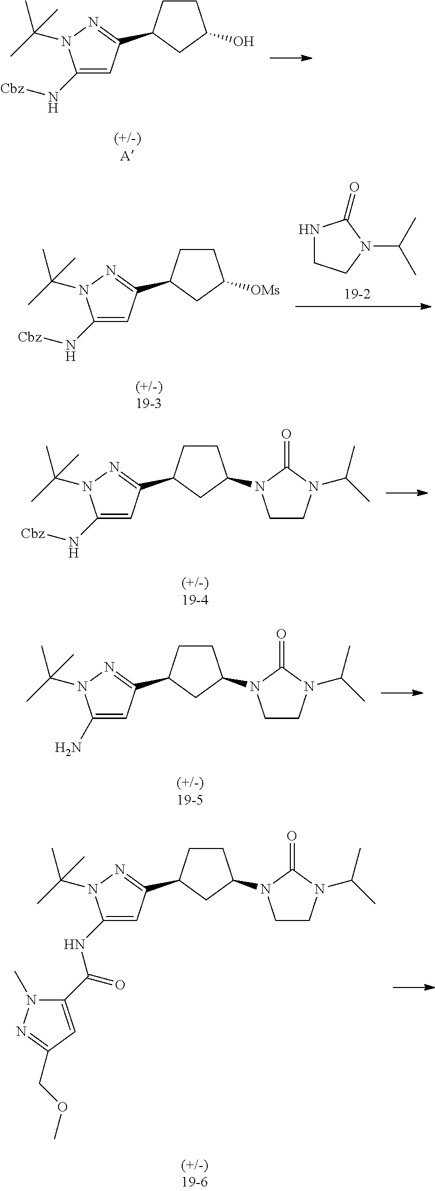

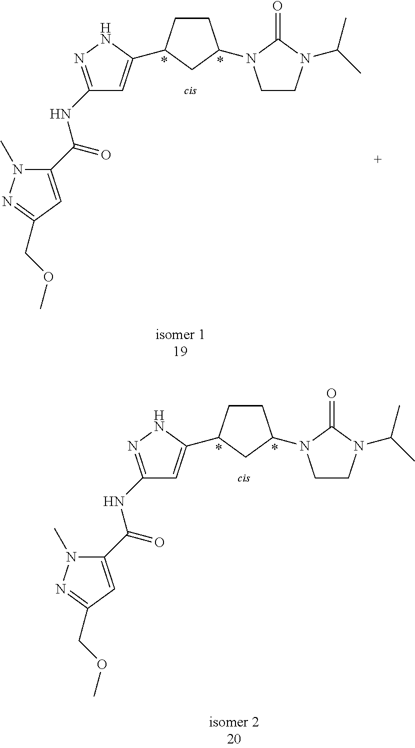

### Step 1 (Synthesis of Compound 19-2)

To a solution of N,N-carbonyldiimidazole (8.0 g, 48.9 mmol, 1.0 eq) dissolved in THE (1.0 L) was added 19-1 (5.0 g, 48.9 mmol, 1.0 eq) at 0° C. The reaction mixture was warmed to 25° C. and stirred for 24 hours before it was concentrated to give a residue which was purified by C18 gel chromatography (acetonitrile:water=5:1) to give 19-2 (3.0 g). LCMS (ESI, m/z): [M+H]⁺=129.2.

### Step 2 (Synthesis of Compound 19-3)

To a mixture of A′ (500.0 mg, 1.4 mmol, 1.0 eq) and TEA (283.1 mg, 2.8 mmol, 2.0 eq) in DCM (10 mL) was added MsCl (320.7 mg, 2.8 mmol, 2.0 eq) dropwise at 0° C. The reaction mixture was stirred at room temperature for 12 hours before it was cooled to 0° C. and quenched with the addition of aqueous saturated NaHCO₃ solution. The mixture was extracted with DCM (×2). The combined organic phase was washed with brine, dried over Na₂SO₄, filtered and concentrated to give a residue which was purified by silica gel chromatography (PE:EtOAc=8:1) to give 19-3 (400.0 mg). LCMS (ESI, m/z): [M+H]⁺=436.0.

### Step 3 (Synthesis of Compound 19-4)

To a solution of 19-2 (737 mg, 5.75 mmol, 5.0 eq) in DMF (15 mL) was added NaH (230 mg, 5.75 mmol, 5.0 eq, 60% in mineral oil) at 15° C. The reaction mixture was stirred at room temperature for 2 hours. Then 19-3 (500 mg, 1.15 mmol, 1.0 eq) was added. The reaction mixture was stirred at 35° C. for 12 hours before it was quenched with water and extracted with dichloromethane:methanol (10:1, ×3). The combined organic phase was washed with brine, dried over Na₂SO₄, filtered and concentrated to give a residue which was purified by column chromatography (PE:EtOAc=2:1) to give 19-4 (300 mg). LCMS (ESI, m/z): [M+H]⁺=468.2.

### Step 4 (Synthesis of Compound 19-5)

To a solution of 19-4 (300.0 mg, 0.64 mmol, 1.0 eq) in THF/ethyl acetate (5 mL/5 mL) was added Pd/C (80 mg, 10% wt). The reaction mixture was stirred under the atmosphere of hydrogen at room temperature for 12 hours before it was filtered through a pad of Celite. The filtrate was concentrated to give 19-5 (200 mg). LCMS (ESI, m/z): [M+H]⁺=334.3.

### Step 5 (Synthesis of Compound 19-6)

To a solution of 19-5 (130 mg, 0.39 mmol, 1.0 eq) in acetonitrile (15 mL) were added C1 (99.5 mg, 0.59 mmol, 1.5 eq), DIEA (126.2 mg, 0.98 mmol, 2.5 eq) and CMPI (179.3 mg, 0.7 mmol, 1.8 eq). The reaction mixture was stirred at 80° C. for 20 hours before it was cooled to room temperature, diluted with water, and extracted with ethyl acetate (×3). The combined organic phase was washed with brine, dried over Na₂SO₄ and concentrated. The residue was purified by prep-TLC (ethyl acetate) to give 19-6 (144 mg).

### Step 6 (Synthesis of Compounds 19 & 20)

The solution of 19-6 (200.0 mg, 0.41 mmol, 1.0 eq) in anhydrous formic acid (8 mL) was stirred at 70° C. for 12 hours. The reaction was concentrated and quenched by the addition of saturated NaHCO₃ solution. The mixture was extracted with ethyl acetate (×2). The combined organic phase was washed with brine, dried over Na₂SO₄ and concentrated. The residue was purified via SFC (column: LUX 5 um Cellulose-3, IPA (0.1% of DEA)/CO₂=15/85) to give compound 19 (isomer 1, 20.3 mg) and compound 20 (isomer 2, 46.1 mg).

The relevant characterization data for compound 19 was as follows: LCMS (ESI, m/z): [M+H]⁺=430.2. ¹H NMR (400 MHz, DMSO-d₆, ppm): δ 12.23 (brs, 1H), 10.72 (brs, 1H), 7.12 (s, 1H), 6.44 (s, 1H), 4.33 (s, 2H), 4.29-4.20 (m, 1H), 4.05 (s, 3H), 3.94-3.85 (m, 1H), 3.27-3.17 (m, 7H), 3.12-3.04 (m, 1H), 2.13-1.97 (m, 2H), 1.82-1.60 (m, 4H), 1.05-1.01 (m, 6H). Chiral purity 99.2% de, retention time 3.317 min. Chiral SFC analysis was performed on a OJ-3 column heated to 35° C., eluted with a mobile phase of CO₂ and IPA (0.1% of DEA), flowing at 2.0 mL/min.

The relevant characterization data for compound 20 was as follows: LCMS (ESI, m/z): [M+H]⁺=430.2. ¹H NMR (400 MHz, DMSO-d₆, ppm): δ 12.29 (brs, 1H), 10.72 (brs, 1H), 7.12 (s, 1H), 6.43 (s, 1H), 4.33 (s, 2H), 4.28-4.21 (m, 1H), 4.05 (s, 3H), 3.95-3.87 (m, 1H), 3.26-3.17 (m, 7H), 3.12-3.04 (m, 1H), 2.12-1.99 (m, 2H), 1.80-1.63 (m, 4H), 1.05-1.01 (m, 6H). Chiral purity 93.98% de, retention time 4.443 min. Chiral SFC analysis was performed on a OJ-3 column heated to 35° C., eluted with a mobile phase of CO₂ and IPA (0.1% of DEA), flowing at 2.0 mL/min.

### Example 10

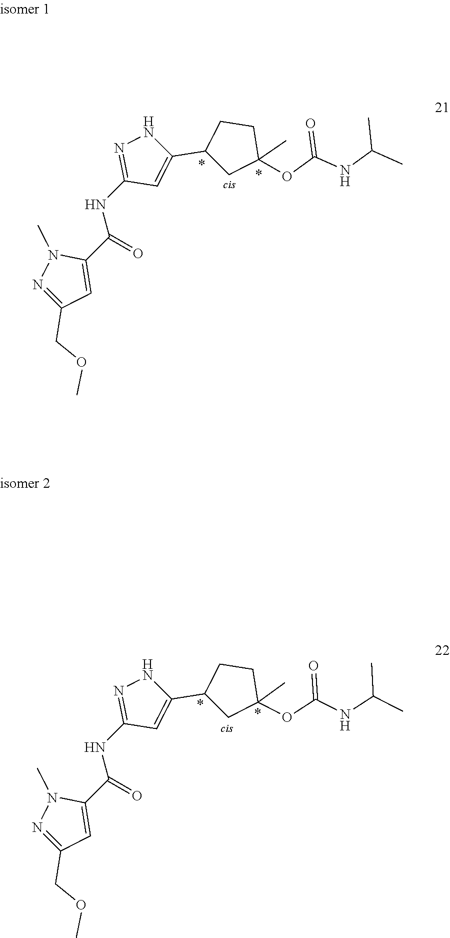 [SMILES: COCc1cc(C(=O)Nc2cc(C3CCC(C)(OC(=O)NC(C)C)C3)[nH]n2)n(C)n1 ; COCc1cc(C(=O)Nc2cc(C3CCC(C)(OC(=O)NC(C)C)C3)[nH]n2)n(C)n1]

### Synthetic Route for Compounds 21 & 22 as Follows:

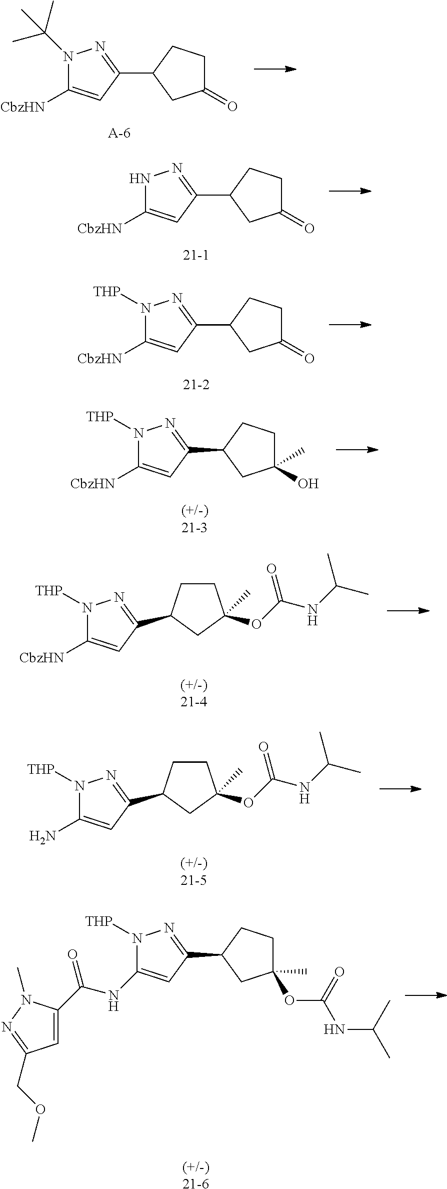

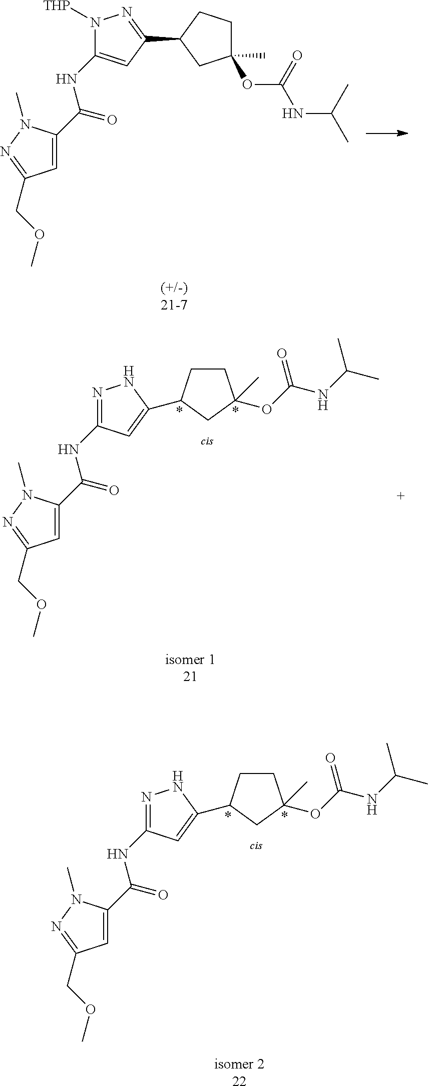

### Step 1 (Synthesis of Compound 21-1)

A solution of benzyl A-6 (2800 mg, 6.13 mmol) in formic acid (80 mL) was heated at 75° C. for 10 hours. The mixture was concentrated, diluted with saturated sodium bicarbonate and extracted with ethyl acetate. The organic phase was concentrated and purified by chromatography on a silica gel column (petroleum ether to petroleum ether/ethyl acetate=1/1) to give 21-1 (1800 mg). LCMS: (ES, m/z): [M+H]⁺=300.2.

### Step 2 (Synthesis of Compound 21-2)

To a solution of 21-1 (1100 mg, 3.68 mmol) in ethyl acetate (50 mL) was added dihydropyran (930 mg, 11.06 mmol) and p-toluenesulfonic acid monohydrate (127 mg, 0.74 mmol). The reaction mixture was stirred at 60° C. for 16 hours before it was cooled and diluted with saturated sodium bicarbonate. The organic phase was concentrated and purified by chromatography on a silica gel column (petroleum ether to petroleum ether/ethyl acetate=1/1) to give 21-2 (1000 mg). LCMS: (ES, m/z): [M+H]⁺=384.3.

### Step 3 (Synthesis of Compound 21-3)

To a solution of 21-2 (940 mg, 2.45 mmol) in THE (50 mL) was added methyllithium (37 mL, 48.10 mmol) at −70° C. under nitrogen. The reaction mixture was stirred at −70° C. for 2 hours before it was quenched with saturated ammonium chloride and extracted with ethyl acetate. The organic phase was concentrated and purified by chromatography on a silica gel column (petroleum ether to petroleum ether/ethyl acetate=1/1) to give 21-3 (550 mg). LCMS: (ES, m/z): [M+H]⁺=400.2.

### Step 4 (Synthesis of Compound 21-4)

To a solution of 21-3 (500 mg, 1.25 mmol) was added n-butyllithium (1.8 mL, 4.50 mmol) at −78° C. under nitrogen. The mixture was stirred at −78° C. for an hour and then 2-isocyanatopropane (427 mg, 5.02 mmol) was added. The reaction mixture was stirred at room temperature for 5 hours before it was extracted with ethyl acetate and washed with water. The organic phase was concentrated and purified by chromatography on a silica gel column (petroleum ether to petroleum ether/ethyl acetate=3/1) to give 21-4 (220 mg, 0.454 mmol). LCMS: (ES, m/z): [M+H]⁺=485.3.

### Step 5 (Synthesis of Compound 21-5)

To a solution of 21-4 (220 mg, 0.454 mmol) in ethyl acetate (10 mL) and tetrahydrofuran (10 mL) was added Pd/C 10% (100 mg, 0.940 mmol). The reaction mixture was stirred at room temperature for 3 hours under hydrogen before it was filtered and concentrated to give 21-5 (159 mg). LCMS: (ES, m/z): [M+H]⁺=351.3.

### Step 6 (Synthesis of Compound 21-6)

To a solution of 21-5 (159 mg, 0.454 mmol) in acetonitrile (40 mL) were added C (160 mg, 0.909 mmol), CMPI (464 mg, 1.82 mmol) and ethyldiisopropylamine (0.307 mL, 1.86 mmol). The reaction mixture was stirred at 80° C. for 4 hours before it was cooled, extracted with ethyl acetate, and washed with water. The organic phase was concentrated and the residue was dissolved in methanol (10 mL) and water (10 mL). The reaction mixture was stirred at room temperature for 1 hour before it was extracted with ethyl acetate and washed with water. The organic phase was concentrated and purified by chromatography on a silica gel column (petroleum ether to petroleum ether/ethyl acetate=1/1) to give 21-6 (120 mg). LCMS: (ES, m/z): [M+H]⁺=503.0.

### Step 7 (Synthesis of Compounds 21 & 22)

To a solution of 21-6 (110 mg, 0.219 mmol) in methanol (20 mL) was added p-toluenesulfonic acid monohydrate (63 mg, 0.331 mmol). The reaction was stirred at room temperature for 24 hours before it was extracted with ethyl acetate and washed with saturated sodium bicarbonate. The organic phase was concentrated, and the residue was purified via SFC [Waters SFC 150, DAICELCHIRALCEL® OZ, 250*25 mm 10 μm, MeOH (0.1% 7M ammonia in MeOH)/supercritical CO₂], then pre-HPLC (acetonitrile/0.05% TFA in water: 5%˜50%) to give compound 21 (isomer 1, 10.3 mg) and compound 22 (isomer 2, 15 mg).

The relevant characterization data for compound 21 was as follows: LCMS (ESI, m/z): [M+H]⁺=419.3; HNMR (400 MHz, METHANOL-d₄, ppm): δ 6.93 (s, 1H), 6.40 (s, 1H), 4.44 (s, 2H), 4.13 (s, 3H), 3.68-3.62 (m, 1H), 3.37 (s, 3H), 3.27-3.23 (m, 1H), 2.33-2.27 (m, 3H), 2.19-2.12 (m, 1H), 1.99-1.89 (m, 1H), 1.83-1.76 (m, 1H), 1.60 (s, 3H), 1.02 (dd, J=6.4 Hz, 4.0 Hz, 6H). Chiral purity 100.0% ee, retention time 4.163 min. Chiral SFC analysis was performed on a Waters UPCC (CA-060), DAICEL CHIRALPAK® OZ, 100*3.0 mm 3 μm column heated to 35° C., eluted with a mobile phase of CO₂ and gradient of 5-40% MeOH (0.1% DEA), flowing at 1.5 mL/min.

The relevant characterization data for compound 22 was as follows: LCMS (ESI, m/z): [M+H]⁺=419.3; HNMR (400 MHz, METHANOL-d₄, ppm): δ 6.93 (s, 1H), 6.40 (s, 1H), 4.44 (s, 2H), 4.13 (s, 3H), 3.68-3.62 (m, 1H), 3.37 (s, 3H), 3.28-3.23 (m, 1H), 2.33-2.27 (m, 3H), 2.19-2.12 (m, 1H), 1.99-1.90 (m, 1H), 1.83-1.76 (m, 1H), 1.60 (s, 3H), 1.02 (dd, J=6.4 Hz, 4.0 Hz, 6H). Chiral purity 97.66% ee, retention time 4.406 min. Chiral SFC analysis was performed on a Waters UPCC (CA-060), DAICEL CHIRALPAK® OZ, 100*3.0 mm 3 μm column heated to 35° C., eluted with a mobile phase of CO₂ and gradient of 5-40% MeOH (0.1% DEA), flowing at 1.5 mL/min.

### Example 11

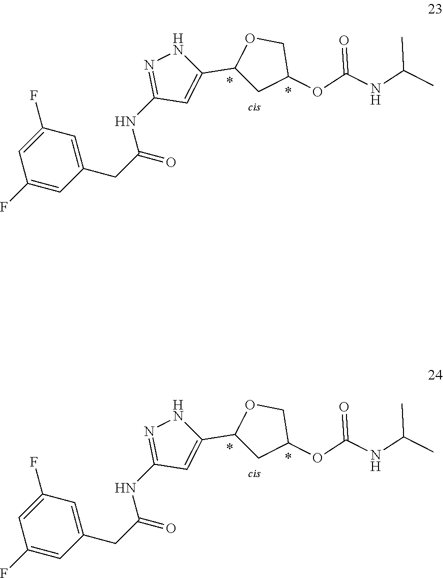 [SMILES: CC(C)NC(=O)OC1COC(c2cc(NC(=O)Cc3cc(F)cc(F)c3)n[nH]2)C1 ; CC(C)NC(=O)OC1COC(c2cc(NC(=O)Cc3cc(F)cc(F)c3)n[nH]2)C1]

### Synthetic Route for Compounds 23 & 24 as Follows:

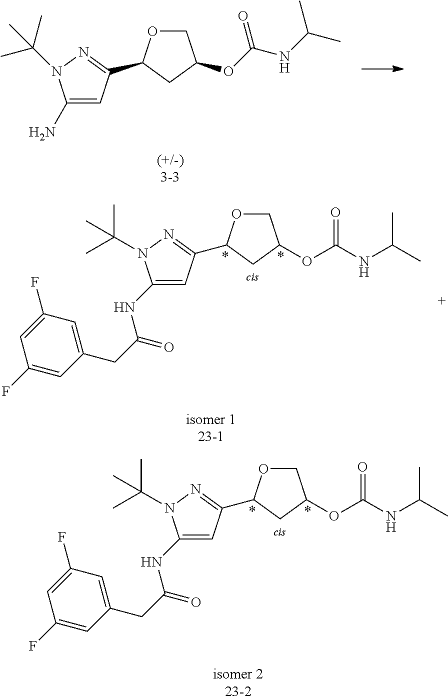 [SMILES: CC(C)NC(=O)O[C@@H]1CO[C@H](c2cc(N)n(C(C)(C)C)n2)C1 ; "(+/-)3-3" ; CC(C)NC(=O)OC1COC(c2cc(NC(=O)Cc3cc(F)cc(F)c3)n(C(C)(C)C)n2)C1 ; "isomer 123-1" ; CC(C)NC(=O)OC1COC(c2cc(NC(=O)Cc3cc(F)cc(F)c3)n(C(C)(C)C)n2)C1 ; "isomer 223-2"]

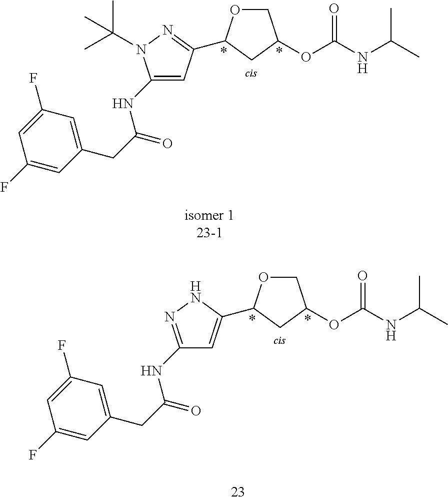 [SMILES: CC(C)NC(=O)OC1COC(c2cc(NC(=O)Cc3cc(F)cc(F)c3)n(C(C)(C)C)n2)C1 ; "isomer 123-1" ; CC(C)NC(=O)OC1COC(c2cc(NC(=O)Cc3cc(F)cc(F)c3)n[nH]2)C1 ; "23"]

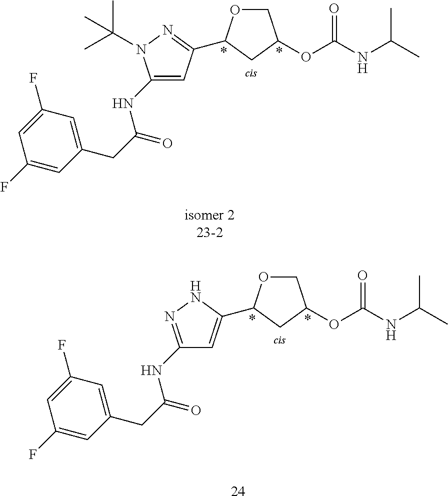 [SMILES: CC(C)NC(=O)OC1COC(c2cc(NC(=O)Cc3cc(F)cc(F)c3)n(C(C)(C)C)n2)C1 ; "isomer 223-2" ; CC(C)NC(=O)OC1COC(c2cc(NC(=O)Cc3cc(F)cc(F)c3)n[nH]2)C1 ; "24"]

### Step 1 (Synthesis of Compounds 23-1, 23-2)

To a mixture of 3-3 (200 mg, 0.644 mmol) and 2-(3,5-difluorophenyl) acetic acid (116.4 mg, 0.677 mmol) in dichloromethane (4 mL) were added DIEA (0.21 mL, 1.29 mmol) and 2-(7-azabenzotriazol-1-yl)-N,N N′,N′-tetramethyluronium hexafluorophosphate (367.5 mg, 0.966 mmol). The reaction mixture was stirred at 25° C. for 7 hours before it was diluted with dichloromethane and washed with saturated sodium bicarbonate solution and water and then brine, dried over anhydrous sodium sulfate, filtered and concentrated in vacuo. The residue was purified by flash chromatography (C-18, 80 g, 0˜50% acetonitrile in H₂O (0.05% TFA)), then separated via SFC [Waters SFC 150, DAICELCHIRALCEL® OZ, 250*25 mm 10 m, MeOH (0.1% 7M ammonia in MeOH)/supercritical CO₂] to give compound 23-1 (isomer 1, 58 mg) and compound 23-2 (isomer 2, 53 mg).

The relevant characterization data for compound 23-1 was as follows: LCMS (ESI, m/z): [M+H]⁺=465.2; Chiral purity 100% ee, retention time 3.280 min. Chiral SFC analysis was performed on a Waters UPCC (CA-060), DAICEL CHIRALPAK® OZ, 100*3.0 mm 3 μm column heated to 35° C., eluted with a mobile phase of CO₂ and gradient of 5-40% MeOH (0.1% DEA), flowing at 1.0 mL/min.

The relevant characterization data for compound 23-2 was as follows: LCMS (ESI, m/z): [M+H]⁺=465.2; Chiral purity 99.2% ee, retention time 4.093 min. Chiral SFC analysis was performed on a Waters UPCC (CA-060), DAICEL CHIRALPAK® OZ, 100*3.0 mm 3 μm column heated to 35° C., eluted with a mobile phase of CO₂ and gradient of 5-40% MeOH (0.1% DEA), flowing at 1.0 mL/min.

### Step 2 (Synthesis of Compounds 23, 24)

A solution of 23-1 (53 mg, 0.114 mmol) in formic acid (2 mL) was heated to 75° C. and stirred for 1 hour. The solvent was removed in vacuo. The residue was purified by Prep-HPLC (eluted with acetonitrile in 0.05% trifluoroacetic acid in water from 5% to 50%) to give 23 (5.7 mg).

The relevant characterization data for compound 23 was as follows: LCMS (ESI, m/z): [M+H]⁺=409.4; ¹H NMR (400 MHz, METHANOL-d₄, ppm): δ 7.00 (m, 2H), 6.90-6.84 (m, 1H), 6.45 (s, 1H), 5.24 (s, 1H), 5.05-4.98 (m, 1H), 4.09-4.02 (m, 1H), 3.99-3.91 (m, 1H), 3.73 (s, 2H), 3.70-3.61 (m, 1H), 2.75-2.66 (m, 1H), 2.18-2.10 (m, 1H), 1.11 (t, J=7.6 Hz, 6H).

Compound 24 was synthesized followed the similar synthetic method for compound 23, The relevant characterization data for compound 24 was as follows: LCMS (ESI, m/z): [M+H]⁺=409.4; HNMR (400 MHz, METHANOL-d₄, ppm): δ 7.02-6.97 (m, 2H), 6.91-6.84 (m, 1H), 6.45 (s, 1H), 5.24 (s, 1H), 5.02 (s, 1H), 4.09-4.03 (m, 1H), 3.99-3.93 (m, 1H), 3.74 (s, 2H), 3.70 (s, 1H), 2.75-2.68 (m, 1H), 2.18-2.11 (m, 1H), 1.12 (t, J=7.2 Hz, 6H).

### Example 12

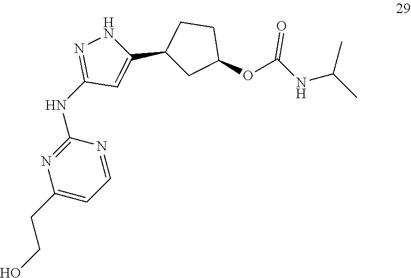 [SMILES: CC(C)NC(=O)O[C@@H]1CC[C@H](c2cc(Nc3nccc(CCO)n3)n[nH]2)C1]

### Synthetic Route for Compound 29 as Follows:

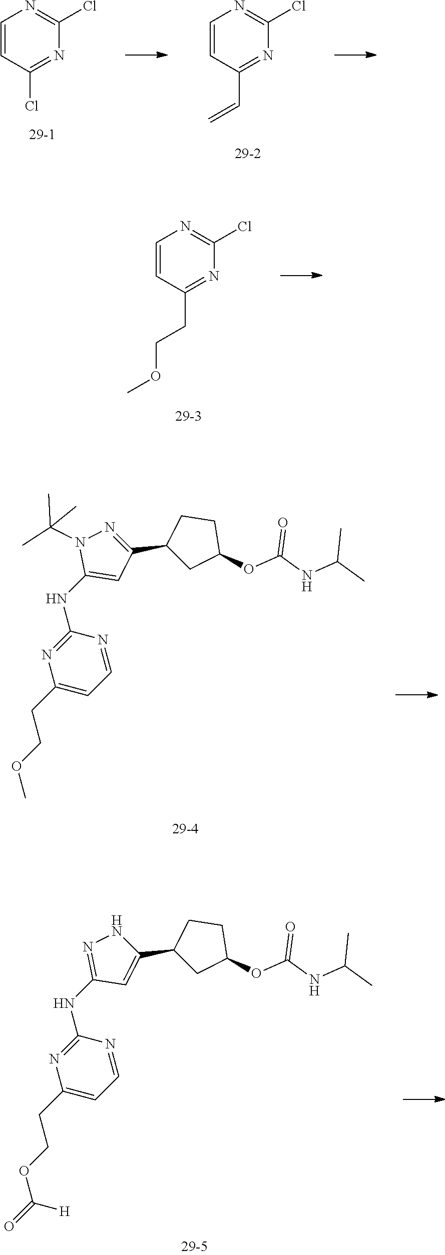

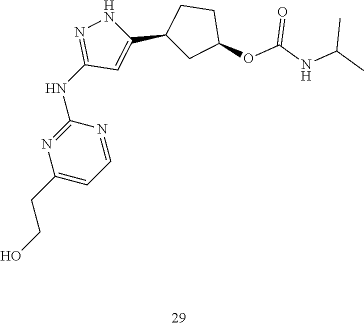 [SMILES: CC(C)NC(=O)O[C@@H]1CC[C@H](c2cc(Nc3nccc(CCO)n3)n[nH]2)C1]

### Step 1 (Synthesis of Compound 29-2)

To a solution of 29-1 (10.0 g, 67.6 mmol, 1.0 eq) in i-PrOH (340 mL) were added Et₃N (6.83 g, 67.6 mmol, 1.0 eq), potassium vinyl trifluoroborate (9.95 g, 74.3 mmol, 1.1 eq) and Pd(dppf)Cl₂ (2.79 g, 3.4 mmol, 0.05 eq). The reaction mixture was stirred at 100° C. for 6 hours under nitrogen atmosphere before it was cooled and concentrated and diluted with water, extracted with EtOAc (×3). The combined organic phase was washed with brine, dried over Na₂SO₄, filtered and concentrated. The residue was purified by silica gel chromatography (PE:EtOAc=10:1) to give 29-2 (5.5 g). LCMS (ESI, m/z): [M+H]⁺=141.1.

### Step 2 (Synthesis of Compound 29-3)

To a solution of 29-2 (5.5 g, 39.3 mmol, 1.0 eq) in toluene (55 mL) was added MeONa (2.1 g, 39.3 mmol, 1.0 eq) at room temperature. The reaction mixture was stirred at room temperature for 15 hours before it was concentrated to give a residue which was diluted with water. The mixture was extracted with EtOAc (×3). The combined organic phase was washed with brine, dried over Na₂SO₄, filtered and concentrated. The residue was purified by silica gel chromatography (PE:EtOAc=3:1) to give 29-3 (1.0 g). LCMS (ESI, m/z): [M+H]⁺=173.1.

### Step 3 (Synthesis of Compound 29-4)

To a solution of 1-2 (100 mg, 0.3 mmol, 1.0 eq) in 2-methyltetrahydrofuran (1 mL) and H₂O (1 mL) were added 29-3 (102 mg, 0.6 mmol, 2.0 eq), Cs₂CO₃ (370 mg, 1.1 mmol, 3.5 eq), BrettPhos (42 mg, 0.1 mmol, 0.24 eq) and Pd₂(dba)₃ (45 mg, 0.1 mmol, 0.15 eq) under nitrogen atmosphere. The reaction mixture was stirred in the sealed tube at 100° C. for 3 hours before it was cooled to room temperature and diluted with water, extracted with EtOAc (×3). The combined organic phase was washed with brine, dried over Na₂SO₄, filtered and concentrated. The residue was purified by silica gel chromatography (DCM:MeOH=40:1) to give 29-4 (100 mg). LCMS (ESI, m/z): [M+H]⁺=445.3.

### Step 4 (Synthesis of Compound 29-5)

The solution of 29-4 (65 mg, 0.2 mmol, 1.0 eq) in anhydrous formic acid (5 mL) was stirred at 70° C. for 15 hours. HPLC showed the reaction was completed. The mixture was concentrated to give a residue which was diluted with water. The aqueous phase was extracted with EtOAc (×3). The combined organic phase was washed with brine, dried over Na₂SO₄, filtered and concentrated to give 29-5 (55 mg, crude), which was used in next step without further purification. LCMS (ESI, m/z): [M+H]⁺=403.2.

### Step 5 (Synthesis of Compound 29)

To a solution of 29-5 (55 mg, crude) in toluene (2 mL) was added MeONa (22 mg, 0.4 mmol). The mixture was stirred at room temperature for 1 hour before it was diluted with water and extracted with EtOAc (×3). The combined organic phase was washed with brine, dried over Na₂SO₄, filtered and concentrated. The residue was purified by prep-HPLC (eluted with acetonitrile in water from 25% to 42%) to give 29 (15 mg).

The relevant characterization data was as follows: LCMS (ESI, m/z): [M+H]⁺=375.2. ¹H NMR (400 MHz, METHANOL-d₄, ppm): δ 8.30 (d, J=5.2 Hz, 1H), 6.75 (d, J=4.8 Hz, 1H), 6.70-6.30 (m, 1H), 5.15-5.08 (m, 1H), 3.96 (t, J=6.4 Hz, 2H), 3.79-3.65 (m, 1H), 3.19-3.10 (m, 1H), 2.88 (t, J=6.4 Hz, 2H), 2.60-2.49 (m, 1H), 2.18-2.08 (m, 1H), 1.98-1.78 (m, 4H), 1.11 (d, J=6.4 Hz, 6H).

### Example 13

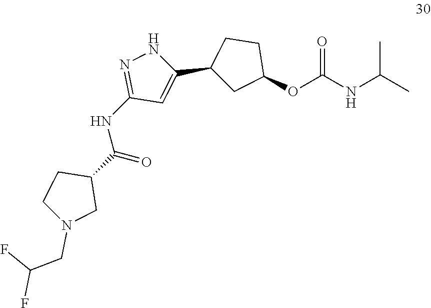 [SMILES: CC(C)NC(=O)O[C@@H]1CC[C@H](c2cc(NC(=O)[C@H]3CCN(CC(F)F)C3)n[nH]2)C1]

### Synthetic Route for Compound 30 as Follows:

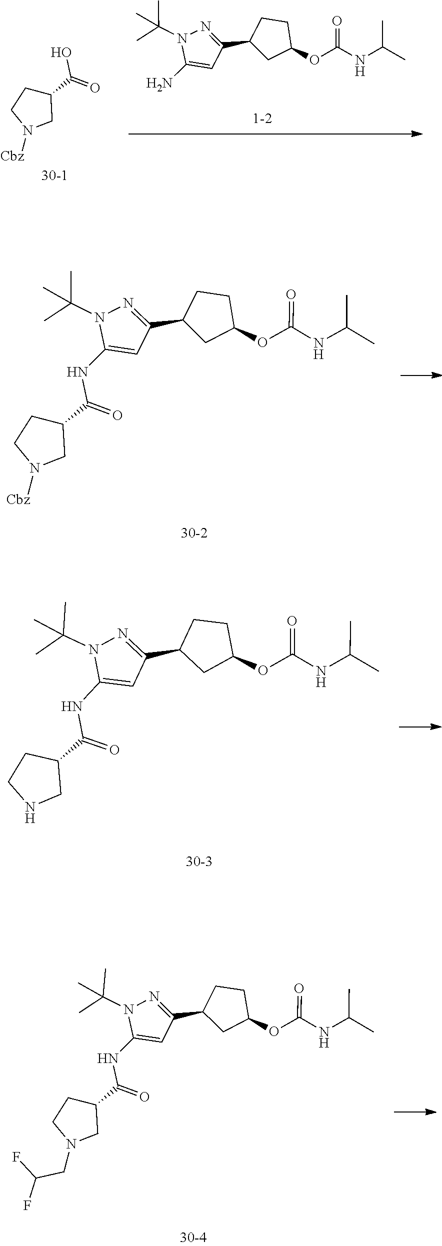 [SMILES: O=C(O)[C@H]1CCN(C(=O)OCc2ccccc2)C1 ; CC(C)NC(=O)O[C@@H]1CC[C@H](c2cc(N)n(C(C)(C)C)n2)C1 ; CC(C)NC(=O)O[C@@H]1CC[C@H](c2cc(NC(=O)[C@H]3CCN(C(=O)OCc4ccccc4)C3)n(C(C)(C)C)n2)C1 ; CC(C)NC(=O)O[C@@H]1CC[C@H](c2cc(NC(=O)[C@H]3CCNC3)n(C(C)(C)C)n2)C1 ; CC(C)NC(=O)O[C@@H]1CC[C@H](c2cc(NC(=O)[C@H]3CCN(CC(F)F)C3)n(C(C)(C)C)n2)C1]

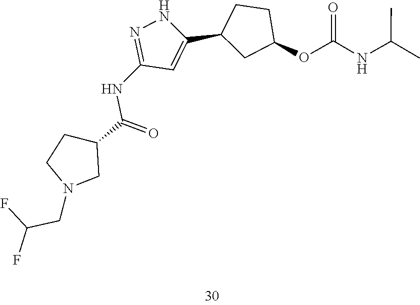 [SMILES: CC(C)NC(=O)O[C@@H]1CC[C@H](c2cc(NC(=O)[C@H]3CCN(CC(F)F)C3)n[nH]2)C1]

### Step 1 (Synthesis of Compound 30-2)

To a solution of 30-1 (194 mg, 0.78 mmol, 2.0 eq) and 1-2 (120 mg, 0.39 mmol, 1.0 eq) in acetonitrile (6 mL) were added and DIEA (101 mg, 0.78 mmol, 2.0 eq) and CMPI (149 mg, 0.58 mmol, 1.5 eq). The reaction mixture was stirred at reflux for 15 hours. HPLC showed the reaction was completed. Then the mixture was cooled to room temperature and partitioned with DCM and water. The aqueous phase was extracted with DCM (×3). The combined organic phase was washed with brine, dried over Na₂SO₄, filtered and concentrated to give a residue which was purified by prep-TLC (DCM:MeOH=20:1) to give 30-2 (150 mg).

LCMS (ESI, m/z): [M+H]⁺=540.3.

### Step 2 (Synthesis of Compound 30-3)

To a solution of 30-2 (150 mg, 1.0 eq) in MeOH (2 mL) was added Pd/C (30 mg, 10% wt). The reaction mixture was stirred under the atmosphere of hydrogen at room temperature for 3 hours. HPLC showed the reaction was completed. The mixture was filtered through a pad of Celite and the filtrate was concentrated to give a residue which was purified by prep-TLC (DCM:MeOH=10:1) to give 30-3 (90 mg). LCMS (ESI, m/z): [M+H]⁺=406.3.

### Step 3 (Synthesis of Compound 30-4)

To a solution of 30-3 (60 mg, 0.15 mmol, 1.0 eq) in DMF (1 mL) were added 1,1-difluoro-2-iodoethane (34 mg, 0.18 mmol, 1.2 eq), DIEA (115 mg, 0.89 mmol, 6 eq). The reaction mixture was stirred at 75° C. for 15 hours before it was cooled and diluted with water, and extracted with EtOAc (×3). The combined organic phase was washed with brine, dried over Na₂SO₄, filtered and concentrated to give a residue which was purified by prep-TLC (DCM:MeOH=30:1) to give 30-4 (36 mg). LCMS (ESI, m/z): [M+H]⁺=470.3.

### Step 4 (Synthesis of Compound 30)

The solution of 30-4 (36 mg, 0.08 mmol, 1.0 eq) in anhydrous formic acid (1.5 mL) was stirred at 70° C. for 4 hours before it was concentrated under reduced pressure. The residue was purified by prep-TLC (DCM:MeOH=10:1) to give 30 (5.9 mg).

The relevant characterization data was as follows: LCMS (ESI, m/z): [M+H]⁺=414.2; ¹HNMR (400 MHz, METHANOL-d₄, ppm): δ 6.34 (s, 1H), 6.14-5.80 (m, 1H), 5.23-5.07 (m, 1H), 3.77-3.65 (m, 1H), 3.15-3.03 (m, 3H), 2.97-2.83 (m, 4H), 2.78-2.72 (m, 1H), 2.56-2.44 (m, 1H), 2.19-1.75 (m, 7H), 1.11 (d, J=6.4 Hz, 6H).

### Example 14

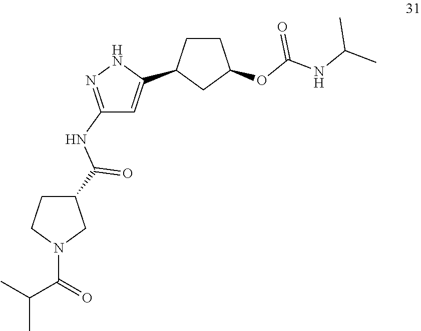 [SMILES: CC(C)NC(=O)O[C@@H]1CC[C@H](c2cc(NC(=O)[C@H]3CCN(C(=O)C(C)C)C3)n[nH]2)C1]

### Synthetic Route for Compound 31 as Follows:

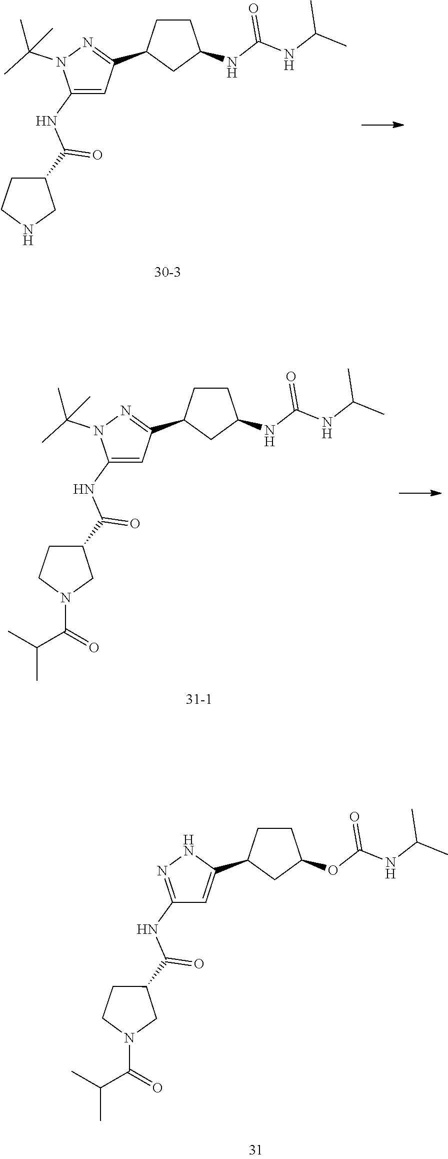

### Step 1 (Synthesis of Compound 31-1)

To a solution of 30-3 (63 mg, 0.16 mmol, 1.0 eq) in THE (1.5 mL) were added TEA (31 mg, 0.31 mmol, 2.0 eq) and isobutyryl chloride (25 mg, 0.23 mmol, 1.5 eq) at 0° C. under N2 atmosphere. The mixture was stirred at room temperature for 3 hours before it was diluted with water, and extracted with EtOAc (×3). The combined organic phase was washed with brine, dried over Na₂SO₄, filtered and concentrated to give 31-1 (75 mg). LCMS (ESI, m/z): [M+H]⁺=476.3.

### Step 2 (Synthesis of Compound 31)

The solution of 31-1 (80 mg, 0.16 mmol, 1.0 eq) in anhydrous formic acid (1.5 mL) was stirred at 70° C. for 2 hours. HPLC showed the reaction was completed. The mixture was concentrated and the residue was purified by prep-TLC (DCM:MeOH=10:1) to give 31 (28.7 mg).

The relevant characterization data was as follows: LCMS (ESI, m/z): [M+H]⁺=420.2; ¹HNMR (400 MHz, DMSO-d₆, ppm): δ 12.05 (brs, 1H), 10.43 (brs, 1H), 6.91 (brs, 1H), 6.31 (s, 1H), 4.98 (s, 1H), 3.80-3.35 (m, 5H), 3.22-3.02 (m, 3H), 2.69-2.59 (m, 1H), 2.16-1.83 (m, 4H), 1.74-1.46 (m, 3H), 1.11-0.92 (m, 12H).

### Example 15

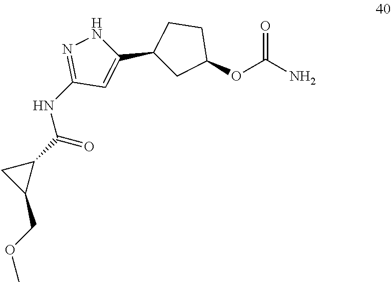 [SMILES: COC[C@H]1C[C@@H]1C(=O)Nc1cc([C@H]2CC[C@@H](OC(N)=O)C2)[nH]n1]

### Synthetic Route for Compound 40 as Follows:

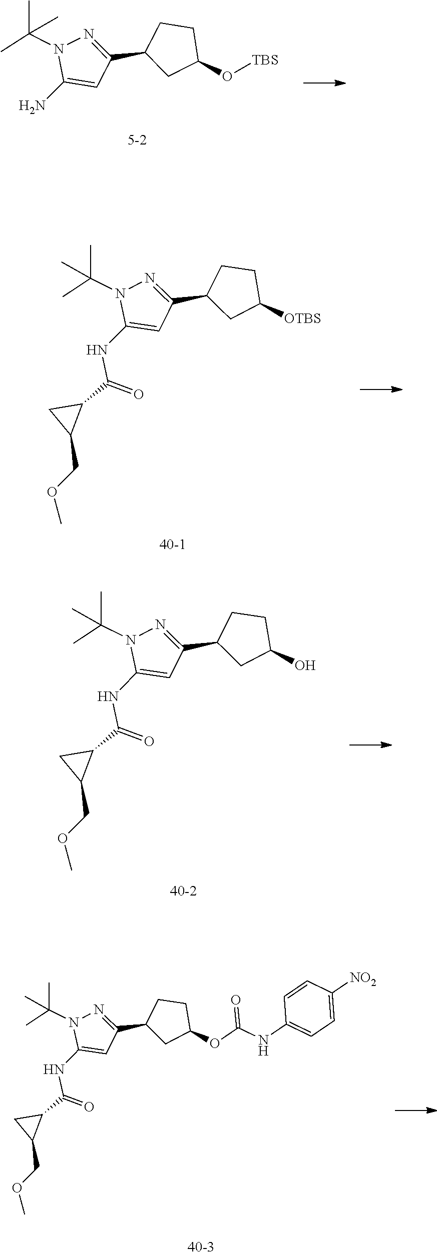

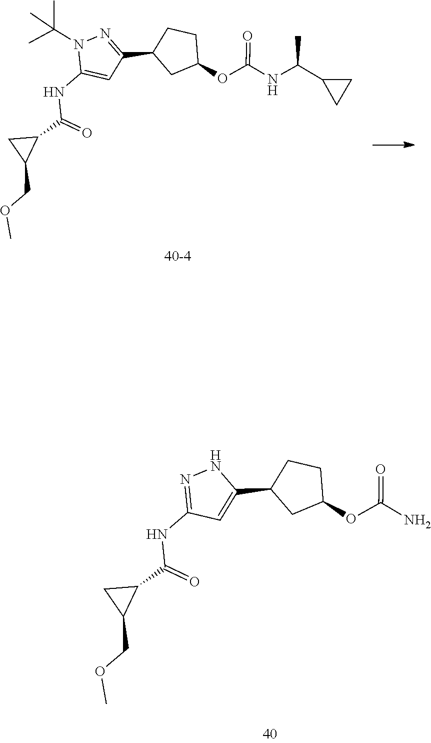 [SMILES: COC[C@H]1C[C@@H]1C(=O)Nc1cc([C@H]2CC[C@@H](OC(=O)N[C@@H](C)C3CC3)C2)nn1C(C)(C)C ; COC[C@H]1C[C@@H]1C(=O)Nc1cc([C@H]2CC[C@@H](OC(N)=O)C2)[nH]n1]

### Step 1 (Synthesis of Compound 40-1)

To a solution of 5-2 (1.3 g, 3.9 mmol, 1.0 eq) and F (752 mg, 5.8 mmol, 1.5 eq) in acetonitrile (15 mL) were added DIEA (995 mg, 7.7 mmol, 2.0 eq) and CMPI (1.5 g, 5.8 mmol, 1.5 eq). The reaction mixture was stirred at 75° C. overnight before it was diluted with DCM, washed with water and brine, dried over Na₂SO₄, concentrated to give crude 40-1 (2.0 g, crude), which was used for next step without further purification. LCMS (ESI, m/z): [M+H]⁺=450.3

### Step 2 (Synthesis of Compound 40-2)

A solution of 40-1 (2.0 g, crude) in formic acid (10 mL) was stirred at room temperature for 1 hour. LCMS showed the reaction was completed. Then the mixture was diluted with DCM, washed with water and brine, dried over Na₂SO₄, filtered and concentrated to give a residue which was purified by silica gel chromatography (DCM MeOH=100:1 to 20:1) to give 40-2 (600 mg). LCMS (ESI, m/z): [M+H]⁺=336.2

### Step 3 (Synthesis of Compound 40-3)

To a solution of 40-2 (600 mg, 1.8 mmol, 1.0 eq) and 4-nitrophenyl chloroformate (541 mg, 2.7 mmol, 1.5 eq) in DCM (18 mL) were added pyridine (424 mg, 5.4 mmol, 3.0 eq) and DMAP (22 mg, 0.2 mmol, 0.1 eq). The reaction mixture was stirred at room temperature for 2 hours before it was diluted with DCM, washed with 1N NaOH solution (×3) and NaHCO₃, dried over Na₂SO₄, filtered and concentrated to give a residue which was purified by silica gel chromatography (PE:EtOAc=3:1 to 1:1) to give 40-3 (320 mg). LCMS (ESI, m/z): [M+H]⁺=501.2

### Step 4 (Synthesis of Compound 40-4)

To a solution of 40-3 (80 mg, 0.16 mmol, 1.0 eq) and (S)-1-cyclopropylethan-1-amine hydrochloride (116 mg, 0.96 mmol, 6.0 eq) in DCM (8 mL) was added DIEA (124 mg, 0.96 mmol, 6.0 eq). The mixture was stirred at room temperature for 6 hours before it was diluted with DCM, washed with water and then brine, dried over Na₂SO₄, concentrated in vacuo. The residue was purified by Prep-TLC (DCM:MeOH=20:1) to give compound 40-4 (40 mg). LCMS (ESI, m/z): [M+H]⁺=447.3

### Step 5 (Synthesis of Compound 40)

The solution of 40-4 (40 mg, 0.46 mmol, 1.0 eq) in formic acid (4 mL) was heated to 100° C. for 1 hour before it was cooled to room temperature, concentrated, and diluted with water, and extracted with DCM (×3). The combined organic layer was washed with brine, dried over Na₂SO₄ and concentrated in vacuo. The crude product was purified by Prep-HPLC (acetonitrile: 0.1% FA in water, 10% to 31%) to afford 40 (13 mg).

The relevant characterization data was as follows: LCMS (ESI, m/z): [M+H]⁺=323.1. ¹HNMR (400 MHz, METHANOL-d₄, ppm): δ 6.31 (s, 1H), 5.13-5.07 (m, 1H), 3.53-3.48 (m, 1H), 3.35 (s, 3H), 3.29-3.25 (m, 1H), 3.19-3.10 (m, 1H), 2.53-2.47 (m, 1H), 2.12-2.06 (m, 1H), 1.95-1.60 (m, 6H), 1.21-1.15 (m, 1H), 0.90-0.83 (m, 1H).

### Example 16

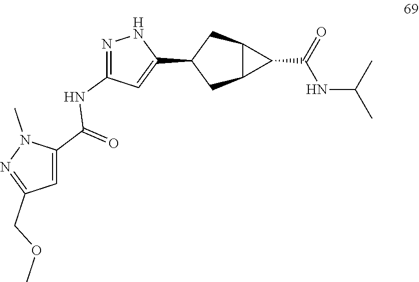 [SMILES: COCc1cc(C(=O)Nc2cc([C@H]3C[C@@H]4[C@H](C3)[C@H]4C(=O)NC(C)C)[nH]n2)n(C)n1]

### Synthetic Route for Compound 69 as Follows:

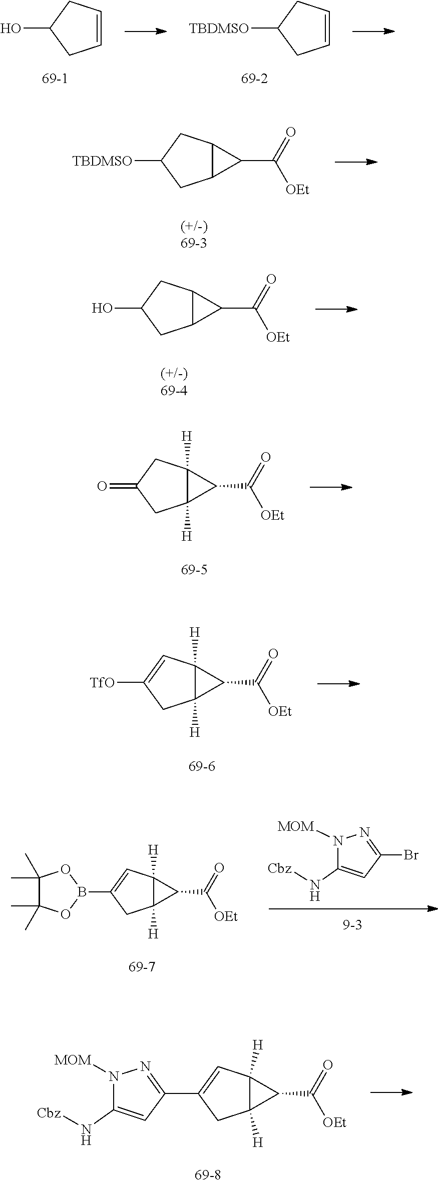

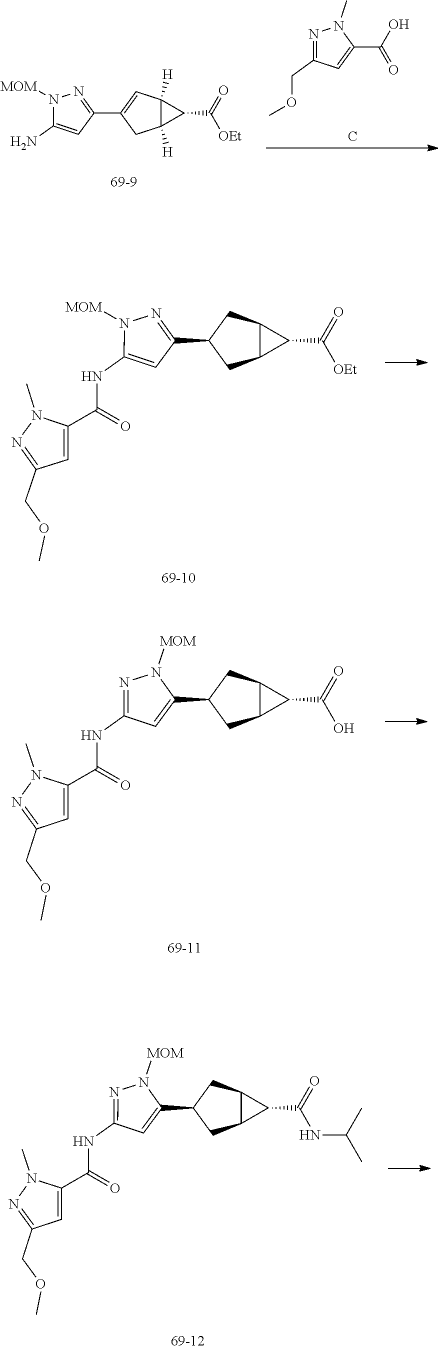

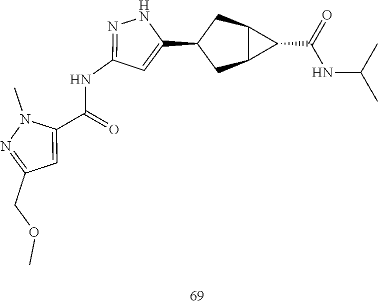 [SMILES: COCc1cc(C(=O)Nc2cc([C@H]3C[C@@H]4[C@H](C3)[C@H]4C(=O)NC(C)C)[nH]n2)n(C)n1]

### Step 1 (Synthesis of Compound 69-2)

To a solution of 69-1 (20 g, 237.756 mmol) in DMF (200 mL) was added imidazole (32.4 g, 475.5 mmol), tert-butyl(chloro)dimethylsilane (43.0 g, 285.3 mmol). The reaction was stirred at room temperature for 16 hours before it was diluted with EtOAc. The organic layer was washed with H₂O, brine, dried over sodium sulfate and concentrated. The residue was purified by column chromatography on silica gel (petroleum ether to petroleum ether/ethyl acetate=1/5) to give 69-2 (40 g).

### Step 2 (Synthesis of Compound 69-3)

To a solution of 69-2 (22 g, 110.9 mmol) and Rhodium (II) acetate dimer (0.5 g, 1.109 mmol) in 1,2-dichloroethane (500 mL) was added a solution of ethyl 2-diazoacetate (15.18 g, 133.1 mmol) in 1,2-dichloroethane (100 mL) dropwise at 25° C. under N2. The reaction mixture was stirred at room temperature for 16 hours before it was filtered through a pad of Celite. The filtrate was concentrated and purified by column chromatography on silica gel (petroleum ether to petroleum ether/ethyl acetate=1/4) to give 69-3 (16 g).

### Step 3 (Synthesis of Compound 69-4)

To a solution of 69-3 (16 g, 56.2 mmol) in THE (100 mL) was added TBAF (29.4 g, 112.5 mmol, 1M in THF), then stirred at 50° C. for 3 hours. Then the reaction was diluted with EtOAc, washed with H₂O and then brine, dried over sodium sulfate and concentrated. The residue was purified by column chromatography on silica gel (petroleum ether to petroleum ether/ethyl acetate=1/5) to give 69-4 (9 g).

### Step 4 (Synthesis of Compound 69-5)

To a solution of 69-4 (9 g, 52.9 mmol) in DCM (200 mL) was added Dess-Martin periodinane (26.9 g, 63.4 mmol) and the reaction was stirred at room temperature for 16 hours. The mixture was filtered through a pad of Celite and the filtrate was treated with aq NaHCO₃ and extracted with DCM. The organic layer was washed with H₂O, dried over sodium sulfate and concentrated. The residue was purified by column chromatography on silica gel (petroleum ether to petroleum ether/ethyl acetate=1/1) to give 65-5 (7 g).

### Step 5 (Synthesis of Compound 69-6)

To a solution of 65-5 (500 mg, 2.97 mmol) in toluene (15 mL) were added DIEA (1150.4 mg, 8.92 mmol), trifluoromethanesulfonic anhydride (2516.3 mg, 8.92 mmol) dropwise. The mixture was stirred at 45° C. for 2 hours and then diluted with EtOAc. The organic layer was washed with H₂O and then brine, dried over sodium sulfate and concentrated. The residue was purified by column chromatography on silica gel (petroleum ether to petroleum ether/ethyl acetate=1/5) to give 69-6 (400 mg).

### Step 6 (Synthesis of Compound 69-7)

To the solution of 69-6 (1200.0 mg, 4.0 mmol) and 4,4,5,5-tetramethyl-2-(tetramethyl-1,3,2-dioxaborolan2-yl)-1,3,2-dioxaborolane (1520.0 mg, 6.0 mmol) in dioxane (20 mL) were added KOAc (1200.0 mg, 12 mmol), 1,1′-bis(diphenylphosphino)ferrocene (112.8 mg, 0.20 mmol) and [1,1′-bis(diphenylphosphino)ferrocene]dichloropalladium(II) (146.2 mg, 0.20 mmol), then stirred at 90° C. for 16 hours under N2. The reaction mixture was cooled to room temperature and diluted with EtOAc. Then the organic layer was washed with H₂O, aq. NaCl, dried over sodium sulfate and concentrated. The residue was purified by column chromatography on silica gel (petroleum ether to petroleum ether/ethyl acetate=1/4) to give 69-7 (560 mg).

### Step 7 (Synthesis of Compound 69-8)

To the solution of 69-7 (560 mg, 2.01 mmol) and 9-3 (684.9 mg, 2.01 mmol) in dioxane (100 mL) and H₂O (10 mL) were added K₂CO₃ (834.7 mg, 6.04 mmol) and [1,1′-bis(diphenylphosphino)ferrocene]dichloropalladium(II) (221.0 mg, 0.302 mmol), then stirred at 95° C. for 16 hours under N2. The mixture was cooled to room temperature and diluted with EtOAc. The organic layer was washed with H₂O and aq. NaCl, dried over sodium sulfate and concentrated. The residue was purified by column chromatography on silica gel (petroleum ether to petroleum ether/ethyl acetate=1/4) to give 69-8 (300 mg). LCMS (ESI, m/z): [M+H]⁺=412.2

### Step 8 (Synthesis of Compound 69-9)

To the solution of 69-8 (300 mg, 0.729 mmol) in THE (15 mL) and EtOAc (15 mL) was added Pd/C 10% (100 mg, 0.940 mmol) at room temperature. The reaction mixture was stirred at room temperature under H₂ for 6 hours before it was filtered a pad of Celite and concentrated to give 69-9 (200 mg), which was used in next step.

### Step 9 (Synthesis of Compound 69-10)

To the solution of C (168.41 mg, 0.841 mmol) and 69-9 (235 mg, 0.841 mmol) in acetonitrile (20 mL) were added CMPI (322.4 mg, 1.26 mmol) and DIEA (325.6 mg, 2.52 mmol) at room temperature under N2, then stirred at 75° C. for 1 hour. The reaction mixture was cooled to room temperature and diluted with EtOAc. The organic layer was then washed with H₂O and aq. NaCl, dried over sodium sulfate and concentrated. The residue was purified by column chromatography on silica gel (petroleum ether to petroleum ether/ethyl acetate=1/2) to give 69-10 (180 mg). LCMS (ESI, m/z): [M+H]⁺=432.2

### Step 10 (Synthesis of Compound 69-11)

To the solution of 69-10 (180 mg, 0.417 mmol) in THE (5 mL) and H₂O (5 mL) was added LiOH·H₂O (52.5 mg, 1.25 mmol) at 0° C. under N2, then stirred at room temperature for 16 hours. Then the reaction was adjusted with 1N HCl to pH=4, diluted with EtOAc. The organic layer was washed with H₂O and aq. NaCl, dried over sodium sulfate and concentrated to give 69-11 (160 mg). LCMS (ESI, m/z): [M+H]⁺=404.2

### Step 11 (Synthesis of Compound 69-12)

To the solution of 69-11 (160 mg, 0.397 mmol) and propan-2-amine (28.13 mg, 0.476 mmol) in DMF (10 mL) were added DIEA (153.5 mg, 1.19 mmol) and HATU (226.2 mg, 0.595 mmol) at room temperature under N2, then stirred at room temperature for 2 hours. Then the reaction was diluted with EtOAc. The organic layer was washed with H₂O and aq. NaCl, dried over sodium sulfate and concentrated. The residue was purified by column chromatography on silica gel (petroleum ether to petroleum ether/ethyl acetate=1/2) to give 69-12 (120 mg).

### Step 12 (Synthesis of Compound 69)

To a solution of 69-12 (60 mg, 0.135 mmol) in EtOH (3 mL) was added 4 M HCl/ethyl acetate (2 mL) at room temperature and stirred at 60° C. for 4 hrs. The reaction was diluted with ethyl acetate and saturated sodium bicarbonate aqueous, extracted with ethyl acetate (×2). Organic layer was dried over sodium sulfate, filtered and concentrated. The residue was purified by prep-HPLC (acetonitrile/0.05% TFA in water: 10%˜35%) to give 69 (24.5 mg).

The relevant characterization data was as follows: LCMS (ESI, m/z): [M+H]⁺=401.2; ¹HNMR (400 MHz, METHANOL-d₄, ppm): δ 6.95 (s, 1H), 6.35 (s, 1H), 4.44 (s, 2H), 4.15 (s, 3H), 3.93-3.87 (m, 1H), 3.53-3.48 (m, 1H), 3.38 (s, 3H), 2.53-2.46 (m, 2H), 2.14-2.10 (m, 2H), 1.86 (s, 2H), 1.32-1.31 (m, 1H), 1.08 (d, J=6.4 Hz, 6H).

### Example 17

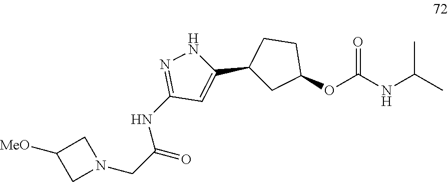 [SMILES: COC1CN(CC(=O)Nc2cc([C@H]3CC[C@@H](OC(=O)NC(C)C)C3)[nH]n2)C1]

### Synthetic Route for Compound 72 as Follows:

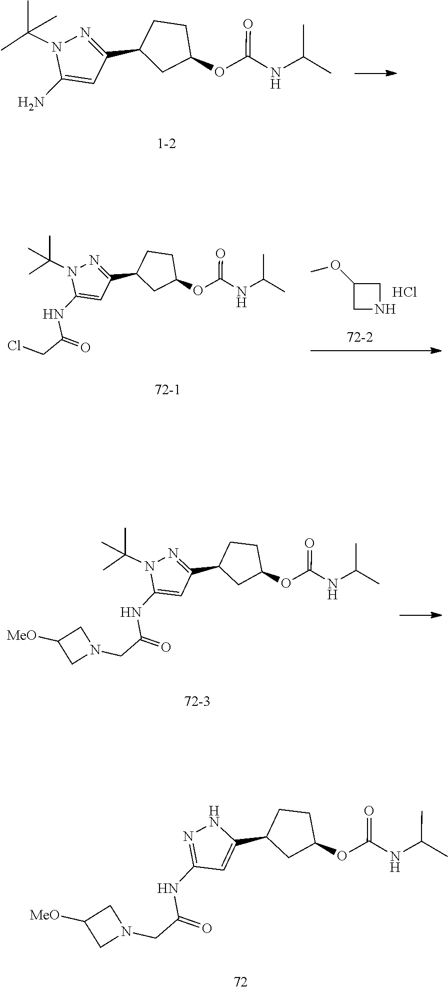

### Step 1 (Synthesis of Compound 72-1)

To a solution of 1-2 (100 mg, 0.32 mmol, 1.0 eq) in THE (2 mL) were added DIEA (93.9 mg, 0.65 mmol, 2.0 eq) and chloroacetyl chloride (47.0 mg, 0.42 mmol, 1.3 eq) at 0° C. The reaction mixture was stirred at room temperature for 3 hours before it was quenched with water. The aqueous phase was extracted with EtOAc (×3). The combined organic phase was washed with brine, dried over Na₂SO₄, filtered and concentrated. The residue was purified by prep-TLC (DCM:MeOH=40:1) to give 72-1 (52 mg). LCMS (ESI, m/z): [M+H]⁺=385.1.

### Step 2 (Synthesis of Compound 72-3)

To a solution of 72-1 (47 mg, 0.12 mmol, 0.25 eq) in acetonitrile (2 mL) was added 72-2 (60.5 mg, 0.49 mmol, 1.0 eq) and K₂CO₃ (135 mg, 0.98 mmol, 2.0 eq). The reaction mixture was stirred at 40° C. for 2 hours before it was concentrated to give a residue which was taken up in water. The mixture was extracted with EtOAc (×3). The combined organic phase was dried over Na₂SO₄, filtered and concentrated to give compound 72-3 (62 mg, crude), which was used in next step without further purification. LCMS (ESI, m/z): [M+H]⁺=436.3

### Step 3 (Synthesis of Compound 72)

The solution of 72-3 (62 mg, crude) in anhydrous formic acid (0.5 mL) was stirred at 70° C. for 4 hours. HPLC showed the reaction was completed. The mixture was concentrated to give a residue, which was purified by prep-TLC (DCM:MeOH=15:1) to give 72 (14.6 mg).

The relevant characterization data was as follows: LCMS (ESI, m/z): [M+H]⁺=380.2; ¹HNMR (300 MHz, METHANOL-d₄, ppm): δ 6.37 (s, 1H), 5.10-5.06 (m, 1H), 4.14-4.10 (m, 1H), 3.84-3.81 (m, 2H), 3.79-3.68 (m, 1H), 3.40 (s, 2H), 3.27 (s, 3H), 3.25-3.20 (m, 2H), 3.18-3.10 (m, 1H), 2.53-2.48 (m, 1H), 2.13-2.06 (m, 1H), 1.93-1.80 (m, 4H), 1.11 (d, J=6.3 Hz, 6H).

### Example 18

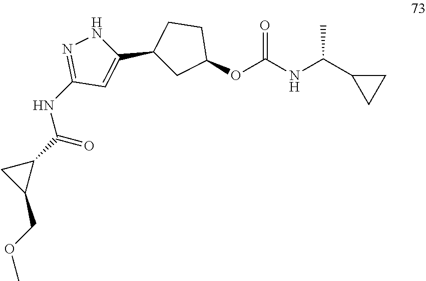 [SMILES: COC[C@H]1C[C@@H]1C(=O)Nc1cc([C@H]2CC[C@@H](OC(=O)N[C@H](C)C3CC3)C2)[nH]n1]

### Synthetic Route for Compound 73 as Follows:

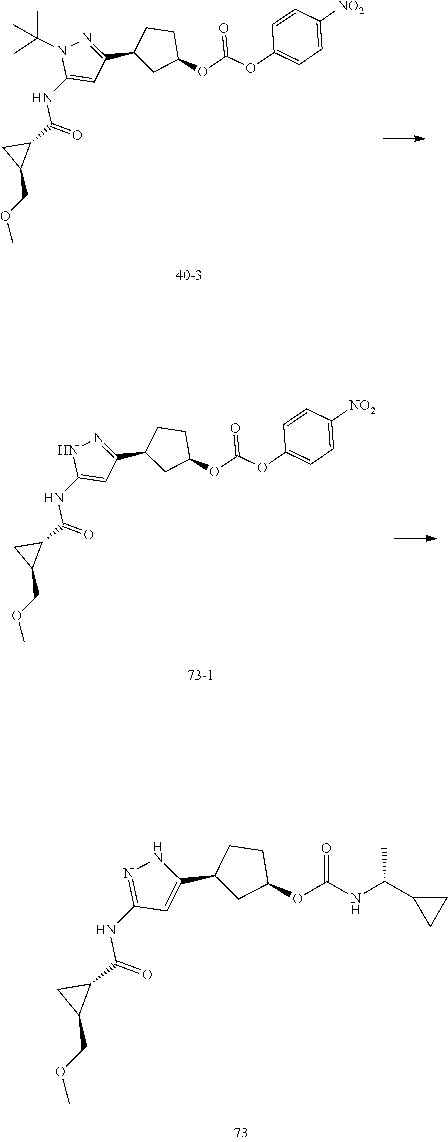 [SMILES: COC[C@H]1C[C@@H]1C(=O)Nc1cc([C@H]2CC[C@@H](OC(=O)Oc3ccc([N+](=O)[O-])cc3)C2)nn1C(C)(C)C ; COC[C@H]1C[C@@H]1C(=O)Nc1cc([C@H]2CC[C@@H](OC(=O)Oc3ccc([N+](=O)[O-])cc3)C2)n[nH]1 ; COC[C@H]1C[C@@H]1C(=O)Nc1cc([C@H]2CC[C@@H](OC(=O)N[C@H](C)C3CC3)C2)[nH]n1]

### Step 1 (Synthesis of Compound 73-1)

A solution of 40-3 (80 mg, 0.16 mmol) in formic acid (2 mL) was heated to 70° C. for 4 hours. HPLC showed the reaction was completed. The resulting mixture was concentrated to give the crude product which was taken up in water and extracted with DCM (×3). The organic extracts were combined, washed with brine, dried over Na₂SO₄ and concentrated in vacuo to give 73-1 (80 mg, crude), which was used in next step without further purification. LCMS (ESI, m/z): [M+H]⁺=445.2

### Step 2 (Synthesis of Compound 73-1)

To a solution of 73-1 (80 mg, crude) and (R)-1-cyclopropylethan-1-amine hydrochloride (117 mg, 0.96 mmol) in THE (3 mL) was added DIEA (124 mg, 0.96 mmol). The reaction mixture was stirred at room temperature for 4 hours before it was diluted with DCM, washed with water and then brine, dried over Na₂SO₄, concentrated in vacuo. The residue was purified by prep-HPLC (acetonitrile with 0.1% FA in water, 35% to 40%) to give 73 (26 mg).

The relevant characterization data was as follows: LCMS (ESI, m/z): [M+H]⁺=391.1. ¹HNMR (400 MHz, METHANOL-d₄, ppm): δ 6.26 (s, 1H), 5.14-5.06 (m, 1H), 3.53-3.46 (m, 1H), 3.35 (s, 3H), 3.28-3.13 (m, 2H), 3.04-2.96 (m, 1H), 2.55-2.45 (m, 1H), 2.18-2.10 (m, 1H), 2.00-1.62 (m, 6H), 1.25-1.20 (m, 1H), 1.17 (d, J=6.8 Hz, 3H), 0.96-0.80 (m, 2H), 0.52-0.37 (m, 2H), 0.35-0.23 (m, 1H), 0.19-0.13 (m, 1H).

### Example 19

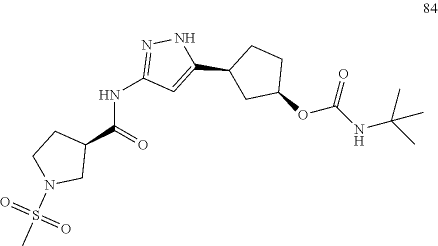 [SMILES: CC(C)(C)NC(=O)O[C@@H]1CC[C@H](c2cc(NC(=O)[C@@H]3CCN(S(C)(=O)=O)C3)n[nH]2)C1]

### Synthetic Route for Compound 84 as Follows:

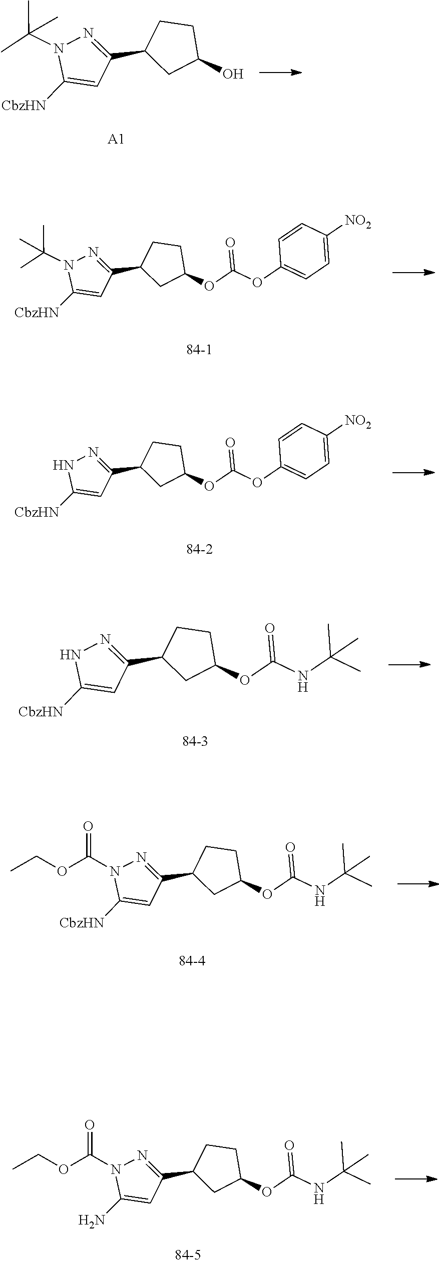

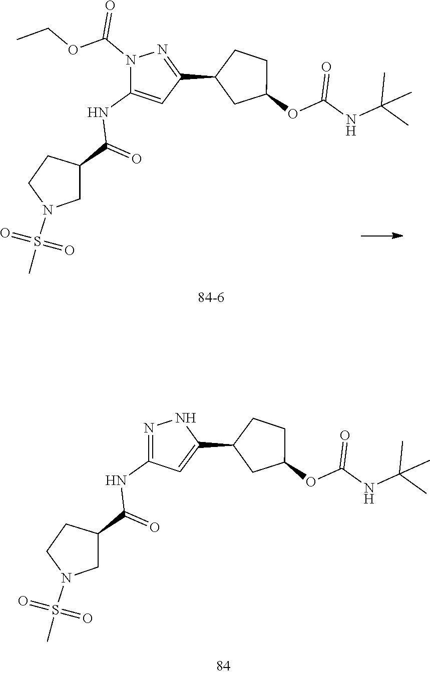 [SMILES: CCOC(=O)n1nc([C@H]2CC[C@@H](OC(=O)NC(C)(C)C)C2)cc1NC(=O)[C@@H]1CCN(S(C)(=O)=O)C1 ; CC(C)(C)NC(=O)O[C@@H]1CC[C@H](c2cc(NC(=O)[C@@H]3CCN(S(C)(=O)=O)C3)n[nH]2)C1]

### Step 1 (Synthesis of Compound 84-1)

To a solution of A1 (500 mg, 1.40 mmol) in DCM (10 mL) were added 4-nitrophenyl chloroformate (480 mg, 2.38 mmol), pyridine (332 mg, 4.20 mmol) and DMAP (34 mg, 0.28 mmol) in sequence at room temperature under nitrogen atmosphere. Then the mixture was stirred for 12 hours at room temperature before it was poured into water and extracted with DCM (×3), the combined organic layers were washed with brine, dried over Na₂SO₄ and concentrated. The residue was purified by prep-TLC (PE/EtOAc=3:1) to give 84-1 (500 mg). LCMS (ESI, m/z): [M+H]⁺=522.8.

### Step 2 (Synthesis of Compound 84-2)

84-1 (400 mg, 0.77 mmol) was dissolved in formic acid (5 mL) at room temperature and stirred at 85° C. for 12 hours. After completion of the reaction, the mixture was concentrated to afford 84-2 (380 mg, crude). LCMS (ESI, m/z): [M+H]⁺=466.8.

### Step 3 (Synthesis of Compound 84-3)

To a solution of 84-2 (420 mg, 0.9 mmol, 1.0 eq) in DCM (8 mL) were added DIEA (580 mg, 4.5 mmol, 5.0 eq) and tert-butylamine (330 mg, 4.5 mmol, 5.0 eq) room temperature. The mixture was stirred for 4 hours at room temperature before it was concentrated to afford 84-3 (1.1 g, crude), which was used for next step without further purification

### Step 4 (Synthesis of Compound 84-4)

To a solution of 84-3 (1.1 g, crude) in DCM (10 mL) were added DIEA (2.0 g, 15.5 mmol) and ethyl chloroformate (1.1 g, 10.1 mmol). The mixture was stirred for 3 hours at room temperature before it was diluted with water and extracted with DCM (×2). The combined organic layer was washed with brine, dried over Na₂SO₄ and concentrated. The residue was purified by silica column chromatography (PE:EtOAc=5:1) to give 84-4 (386 mg). LCMS (ES, m/z): [M+H]⁺=473.1.

### Step 5 (Synthesis of Compound 84-5)

To a solution of 84-4 (380 mg, 0.8 mmol) in EtOAc (10 mL) was added Pd/C (76 mg, 10% wt) and the resulting mixture was stirred at room temperature for 3 hours under the atmosphere of hydrogen. HPLC showed the reaction was completed. The mixture was filtered through a pad of Celite and concentrated to give 84-5 (210 mg). LCMS (ESI, m/z): [M+H]⁺=339.1.

### Step 6 (Synthesis of Compound 84-6)

To a solution of 84-5 (100 mg, 0.30 mmol, 1.0 eq) and D1 (86 mg, 0.45 mmol, 1.5 eq) in acetonitrile (3 mL) were added DIEA (120 mg, 0.9 mmol, 3.0 eq) and CMPI (113 mg, 0.45 mmol, 1.5 eq). The mixture was stirred at 80° C. overnight before it was cooled to room temperature and concentrated in vacuo. The residue was purified by prep-TLC (DCM:MeOH=20:1) to give 84-6 (100 mg). LCMS (ES, m/z): [M+H]⁺=514.2.

### Step 7 (Synthesis of Compound 84)

To a solution of 84-6 (100 mg, 0.2 mmol, 1.0 eq) in MeOH (2 mL) was added TEA (0.8 g, 7.9 mmol, 40.0 eq) at room temperature. The mixture stirred at 40° C. overnight before it was concentrated. The residue was purified by prep-HPLC (acetonitrile with water, 30% to 51%) to give 84 (49 mg).

The relevant characterization data was as follows: LCMS (ESI, m/z): [M+H]⁺=442.0; ¹HNMR (400 MHz, METHANOL-d₄, ppm): δ 6.35 (s, 1H), 5.10-5.03 (m, 1H), 3.63-3.52 (m, 2H), 3.48-3.39 (m, 2H), 3.28-3.23 (m, 1H), 3.17-3.13 (m, 1H), 292 (s, 3H), 2.55-2.45 (m, 1H), 2.32-2.18 (m, 2H), 2.15-2.06 (m, 1H), 1.98-1.72 (m, 4H), 1.28 (s, 9H).

### Example 20

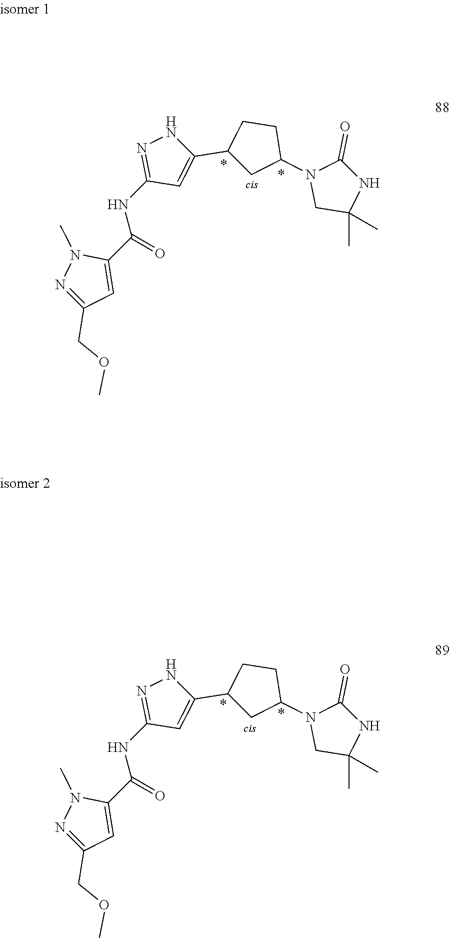 [SMILES: COCc1cc(C(=O)Nc2cc(C3CCC(N4CC(C)(C)NC4=O)C3)[nH]n2)n(C)n1 ; COCc1cc(C(=O)Nc2cc(C3CCC(N4CC(C)(C)NC4=O)C3)[nH]n2)n(C)n1]

### Synthetic Route for Compounds 88 & 89 as Follows:

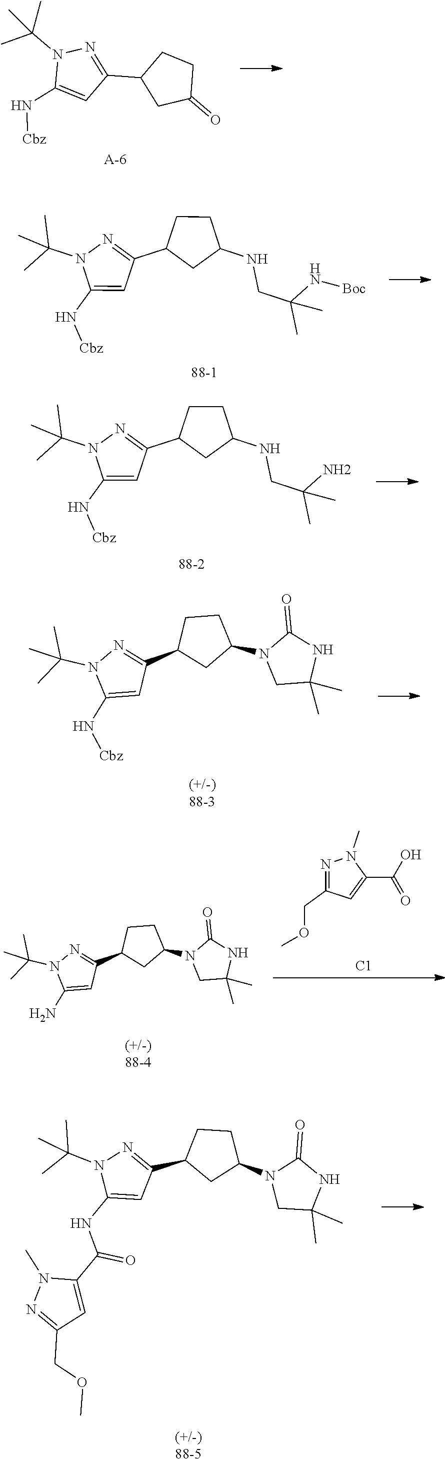

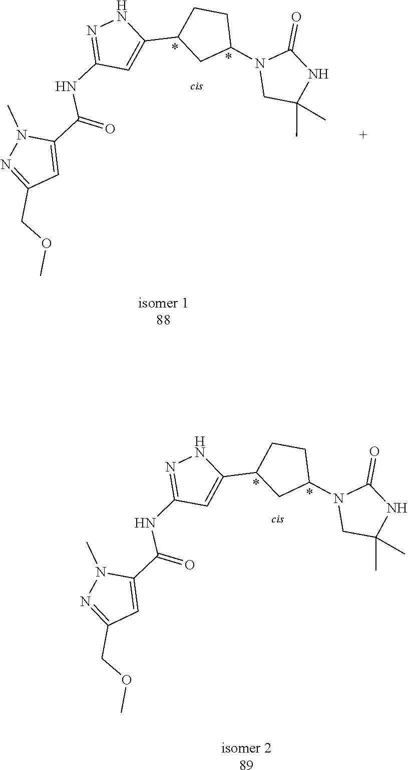 [SMILES: COCc1cc(C(=O)Nc2cc(C3CCC(N4CC(C)(C)NC4=O)C3)[nH]n2)n(C)n1 ; "isomer 188" ; COCc1cc(C(=O)Nc2cc(C3CCC(N4CC(C)(C)NC4=O)C3)[nH]n2)n(C)n1 ; "isomer 289"]

### Step 1 (Synthesis of Compound 88-1)

To a solution of A-6 (900 mg, 2.5 mmol, 1.0 eq) and tert-butyl (1-amino-2-methylpropan-2-yl)carbamate (1.4 g, 7.6 mmol, 3.0 eq) in 1,2-dichloroethane (9 mL) was added HOAc (456 mg, 7.6 mmol, 3.0 eq). The reaction mixture was stirred at room temperature for 1 hour. Then sodium triacetoxyborohydride (1.6 g, 7.6 mmol, 3.0 eq) was added and stirred at room temperature overnight. LCMS showed the reaction was completed. The mixture was diluted with DCM, washed with water and brine, dried over Na₂SO₄, filtered and concentrated. The residue was purified by silica gel chromatography (DCM:MeOH=100:1-30:1) to give 88-1 (1.5 g). LCMS (ESI, m/z): [M+H]⁺=528.3.

### Step 2 (Synthesis of Compound 88-2)

To a solution of 88-1 (1.5 g, 2.8 mmol, 1.0 eq) in DCM (15 mL) was added TFA (4.9 g, 42.6 mmol, 15.0 eq). The reaction mixture was stirred at room temperature for 4 hours before it was concentrated to give 88-2 (3.2 g, crude), which was used in next step without further purification. LCMS (ESI, m/z): [M+H]⁺=428.3

### Step 3 (Synthesis of Compound 88-3)

To a solution of 88-2 (3.2 g, 7.5 mmol, 1.0 eq, crude) in THE (32 mL) were added DIEA (14.5 g, 112.2 mmol, 15.0 eq) and N,N-carbonyldiimidazole (18.2 g, 112.2 mmol, 15.0 eq). The reaction mixture was stirred at 40° C. overnight before it was diluted with DCM, washed with water and brine, dried over Na₂SO₄, filtered and concentrated. The residue was purified by silica gel chromatography (PE:EtOAc=3: 1-1:1) and Prep-HPLC (acetonitrile with 0.11% FA in water, 40% to 51%) to give 88-3 (300 mg).

The relevant characterization data was as follows: LCMS (ESI, m/z): [M+H]⁺=454.4. ¹HNMR (400 MHz, CDCl₃, ppm): δ 7.55-7.28 (m, 5H), 6.64 (s, 1H), 6.04 (s, 1H), 5.18 (s, 2H), 4.50-4.40 (m, 1H), 3.20-3.00 (m, 3H), 2.22-2.13 (m, 1H), 2.10-1.98 (m, 2H), 1.94-1.60 (m, 5H), 1.56 (s, 9H), 1.25 (s, 6H).

### Step 4 (Synthesis of Compound 88-4)

To a solution of 88-3 (300 mg, 0.66 mmol, 1.0 eq) in EtOH (3 mL) was added Pd/C (60 mg, 10% wt) under nitrogen atmosphere. The reaction mixture was stirred under hydrogen atmosphere at room temperature for 2 hours before it was filtered through a pad of Celite and concentrated to give 88-4 (180 mg). LCMS (ESI, m/z): [M+H]⁺=320.4

### Step 5 (Synthesis of Compound 88-5)

To a solution of 88-4 (150 mg, 0.47 mmol, 1.0 eq) and C1 (120 mg, 0.70 mmol, 1.5 eq) in acetonitrile (15 mL) were added DIEA (121 mg, 0.94 mmol, 2.0 eq) and CMPI (180 mg, 0.70 mmol, 1.5 eq). The reaction mixture was stirred at 78° C. for 3 hours before it was cooled, diluted with DCM, washed with water and brine, dried over Na₂SO₄, and concentrated in vacuo. The crude product was purified by Prep-TLC (DCM:MeOH=20:1) to give 88-5 (220 mg). LCMS (ESI, m/z): [M+H]⁺=472.3

### Step 6 (Synthesis of Compounds 88 & 89)

A solution of 88-5 (220 mg, 0.46 mmol, 1.0 eq) in formic acid (5 mL) was heated to 90° C. for 2 hours. HPLC showed the reaction was completed. The resulting mixture was concentrated and the residue was diluted with water and extracted with DCM (×3). The organic phase was washed with brine, dried over Na₂SO₄ and concentrated in vacuo. The crude product was purified by Prep-TLC (DCM:MeOH=10:1), then separated via chiral SFC (column: OJ-3, MeOH:CO₂=10:90) to give compound 88 (isomer 1, 39 mg) and compound 89 (isomer 2, 52 mg).

The relevant characterization data for compound 88 was as follows: LCMS (ESI, m/z): [M+H]⁺=416.1. ¹HNMR (400 MHz, METHANOL-d₄, ppm): δ 6.93 (s, 1H), 6.45 (s, 1H), 4.44 (s, 2H), 4.42-4.30 (m, 1H), 4.13 (s, 3H), 3.38 (s, 3H), 3.24 (s, 2H), 3.25-3.10 (m, 1H), 2.28-2.21 (m, 1H), 2.19-2.12 (m, 1H), 1.98-1.91 (m, 1H), 1.88-1.72 (m, 3H), 1.29 (s, 6H). Chiral purity 98.22% de, retention time: 5.721 min. Chiral SFC analysis was performed on a Waters UPCC, column: DAICELCHIRALCEL®® OJ-3, 250*4.6 mm 3 μm, column heated to 35° C., eluted with a mobile phase of CO₂ and 10% MeOH, flowing at 2.0 mL/min.

The relevant characterization data for compound 89 was as follows: LCMS (ESI, m/z): [M+H]⁺=416.1. ¹HNMR (400 MHz, METHANOL-d₄, ppm): δ 6.93 (s, 1H), 6.45 (s, 1H), 4.44 (s, 2H), 4.41-4.33 (m, 1H), 4.13 (s, 3H), 3.38 (s, 3H), 3.24 (s, 2H), 3.21-3.13 (m, 1H), 2.28-2.21 (m, 1H), 2.18-2.12 (m, 1H), 1.98-1.91 (m, 1H), 1.87-1.73 (m, 3H), 1.29 (s, 6H). Chiral purity 95.34% de, retention time: 8.716 min. Chiral SFC analytical method: Waters UPCC, column: DAICELCHIRALCEL®® OJ-3, 250*4.6 mm 3 μm, column heated to 35° C., eluted with a mobile phase of CO₂ and 10% MeOH, flowing at 2.0 mL/min.

### Example 21

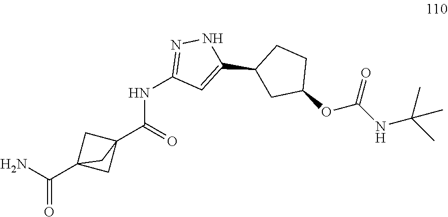 [SMILES: CC(C)(C)NC(=O)O[C@@H]1CC[C@H](c2cc(NC(=O)C34CC(C(N)=O)(C3)C4)n[nH]2)C1]

### Synthetic Route for Compound 110 as Follows:

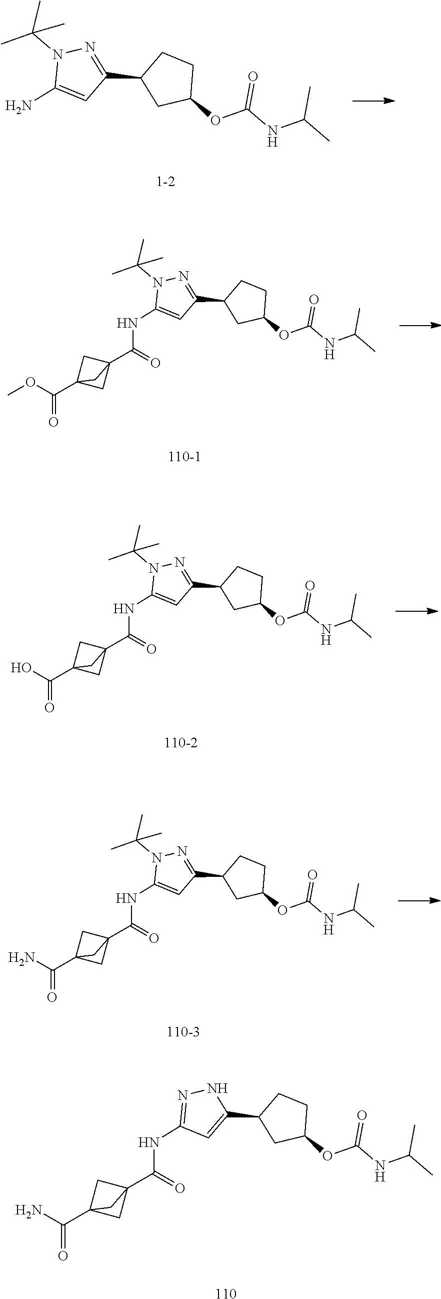

### Step 1 (Synthesis of Compound 110-1)

To a solution of 1-2 (100 mg, 0.32 mmol) in acetonitrile (2 mL) were added 3-(methoxycarbonyl)bicyclo[1.1.1]pentane-1-carboxylic acid (66 mg, 0.39 mmol), DIEA (84 mg, 0.6 mmol, 2.0 eq) and CMPI (110 mg, 0.43 mmol). The reaction mixture was stirred at 80° C. for 15 hours before it was cooled to room temperature, quenched with H₂O, and extracted with DCM (×3). The combined organic phase was washed with brine, dried over Na₂SO₄, filtered and concentrated. The residue was purified by silica gel chromatography (DCM:MeOH=10:1) to give 110-1 (110 mg). LCMS (ESI, m/z): [M+H]⁺=461.2.

### Step 2 (Synthesis of Compound 110-2)

To a solution of 110-1 (96 mg, 0.21 mmol) in THE (1 mL) was added LiOH·H₂O (13 mg, 0.31 mmol) and H₂O (1 mL). The reaction mixture was stirred at room temperature for 1 hour before it was concentrated to give a residue. To the residue was added water, and the mixture was extracted with EtOAc (×2). The organic phase was discarded and the aqueous phase was acidified to pH 2-3 with aqueous 5% citric acid solution which was extracted with DCM (3). The combined organic phase was dried over Na₂SO₄, filtered and concentrated to give compound 110-2 (100 mg, crude). LCMS (ESI, m/z): [M+H]⁺=447.1.

### Step 3 (Synthesis of Compound 110-3)

To a solution of 110-2 (100 mg, crude) in THE (2 mL) were added NH₄Cl (18 mg, 0.33 mmol), 1-ethyl-3-(3-dimethylaminopropyl)carbodiimide hydrochloride (61 mg, 0.33 mmol), 1H-benzo[d][1,2,3]triazol-1-ol (45 mg, 0.33 mmol) and TEA (65 mg, 0.67 mmol) in sequence. The reaction mixture was stirred at room temperature for 1 hour before it was quenched with H₂O and extracted with EtOAc (×3). The combined organic phase was washed with brine, dried over Na₂SO₄, filtered and concentrated. The residue was purified by silica gel chromatography (DCM:MeOH=10:1) to give 110-3 (65 mg). LCMS (ESI, m/z): [M+H]⁺=446.3.

### Step 4 (Synthesis of Compound 110)

The solution of 110-3 (65 mg, 0.15 mmol) in anhydrous formic acid (5 mL) was stirred at 70° C. for 15 hours. HPLC showed the reaction was completed. Then the mixture was concentrated to give a residue which was purified by prep-HPLC (acetonitrile with 0.1% FA in water, 15% to 37%) to give 110 (22.5 mg).

The relevant characterization data was as follows: LCMS (ESI, m/z): [M+H]⁺=390.1; ¹H NMR (400 MHz, DMSO-d₆, ppm): δ 12.08 (brs, 1H), 10.13 (brs, 1H), 7.29 (brs, 1H), 7.05-6.87 (m, 2H), 6.28 (s, 1H), 5.03-4.95 (m, 1H), 3.65-3.50 (m, 1H), 3.12-2.95 (m, 1H), 2.57-2.38 (m, 1H), 2.13 (s, 6H), 2.06-1.94 (m, 1H), 1.94-1.83 (m, 1H), 1.78-1.50 (m, 3H), 1.02 (d, J=6.4 Hz, 6H).

### Example 22

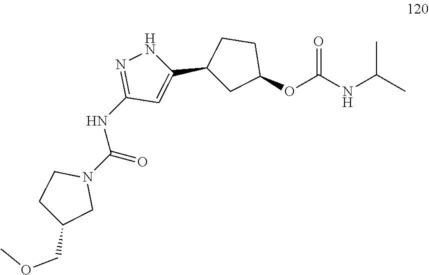 [SMILES: COC[C@@H]1CCN(C(=O)Nc2cc([C@H]3CC[C@@H](OC(=O)NC(C)C)C3)[nH]n2)C1]

### Synthetic Route for Compound 120 as Follows:

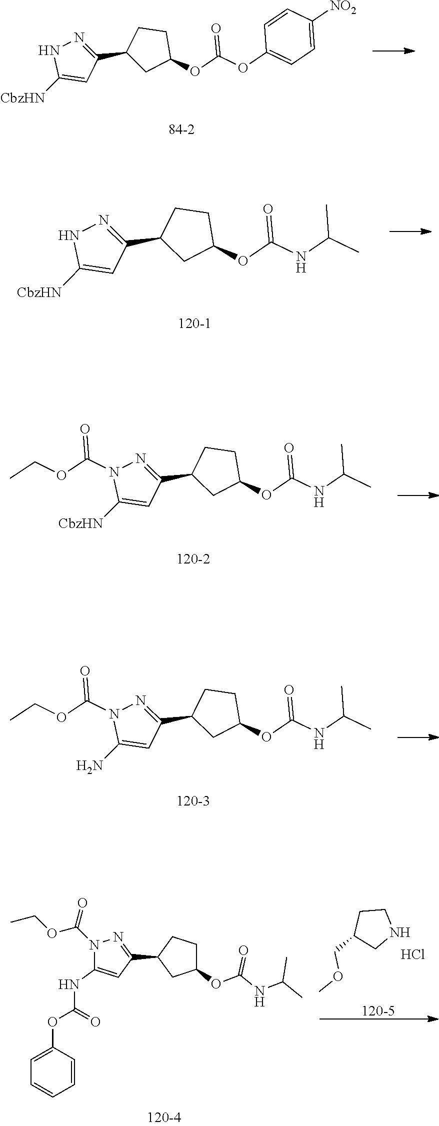

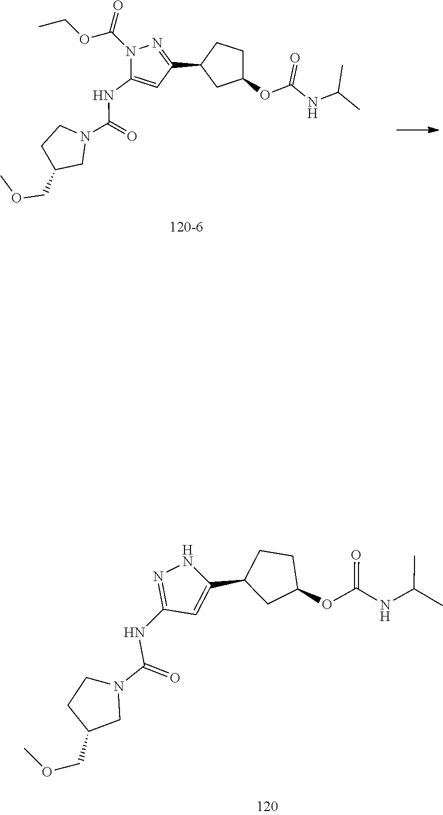

### Step 1 (Synthesis of Compound 120-1)

To a solution of 84-2 (380 mg, crude) in DCM (8 mL) at room temperature were added DIEA (1.0 g, 8.1 mmol) and isopropylamine (240 ng, 4.05 mmol). Then the mixture stirred for 2 hours at same temperature. After completion of the reaction, the reaction mixture was poured into water and the mixture was extracted with DCM (×3). The combined organic layers were washed with brine, dried over Na₂SO₄ and concentrated to afford 120-1 (320 mg, crude). LCMS (ESI, m/z): [M+H]⁺=387.0.

### Step 2 (Synthesis of Compound 120-2)

To a solution of 120-1 (320 mg) in DCM (6 mL) were added DIEA (1.1 g, 8.30 mmol) and ethyl chloroformate (392 mg, 4.15 mmol) at room temperature. Then the mixture was stirred for 2 hours at the same temperature. After completion of the reaction, the reaction mixture was poured into water and the mixture was extracted with DCM (×3). The combined organic layers were washed with brine, dried over Na₂SO₄ and concentrated. The residue was purified by column chromatography on silica gel eluted with (PE/EtOAc=10:1) to give 120-2 (150 mg). LCMS (ESI, m/z): [M+H]⁺=458.8;

### Step 3 (Synthesis of Compound 120-3)

To a solution of 120-2 (150 mg, 0.33 mmol, 1.0 eq) in EtOH (3 mL) was added Pd/C (75 mg, 10% wt) at room temperature, then the mixture was stirred under 50 Psi of hydrogen pressure for 12 hours at room temperature. After completion of the reaction, the mixture was filtered through a Celite pad, and the filtrate was concentrated. The residue was purified by prep-TLC (PE/EtOAc=3:1) to give 120-3 (70 mg). LCMS (ESI, m/z): [M+H]⁺=325.1.

### Step 4 (Synthesis of Compound 120-4)

To a solution of 120-3 (50 mg, 0.15 mmol) in DCM (3 mL) were added phenyl chloroformate (117 mg, 0.75 mmol) and 2,6-lutidine (48 mg, 0.45 mmol) at room temperature. Then the mixture was stirred for 12 hours at same temperature. After completion of the reaction, the reaction mixture was poured into water and the mixture was extracted with DCM (×3). The combined organic layers were washed with brine, dried over Na₂SO₄ and concentrated. The residue was purified by prep-TLC (PE/EtOAc=2:1) to give 120-4 (60 mg). LCMS (ESI, m/z): [M+H]⁺=444.8.

### Step 5 (Synthesis of Compound 120-6)

To a solution of 120-4 (60 mg, 0.14 mmol, 1.0 eq) in DMSO (2 mL) were added 120-5 (57 mg, 0.21 mmol, 1.5 eq) and DIEA (53 mg, 0.42 mmol, 3.0 eq) at room temperature. Then the mixture was stirred for 2 hours at room temperature. After completion of the reaction, the reaction mixture was poured into water and the mixture was extracted with EtOAc (×3). The combined organic layers were washed with brine, dried over Na₂SO₄ and concentrated. The residue was purified by prep-TLC (PE/EtOAc=1:1) to give 120-6 (38 mg). LCMS (ESI, m/z): [M+H]⁺=465.8.

### Step 6 (Synthesis of Compound 120)

To a solution of 120-6 (60 mg, 0.08 mmol, 1.0 eq) in MeOH (1 mL) was added TEA (25 mg, 0.25 mmol, 3.0 eq) at room temperature. Then the mixture stirred for 3 hours at room temperature. After completion of the reaction, the reaction mixture was poured into water and the mixture was extracted with EtOAc (×3). The combined organic layers were washed with brine, dried over Na₂SO₄ and concentrated. The residue was purified by prep-TLC (EtOAc) to give 120 (20.0 mg).

The relevant characterization data was as follows: LCMS (ESI, m/z): [M+H]⁺=394.2; ¹HNMR (400 MHz, METHANOL-d₄, ppm): δ 6.15 (s, 1H), 5.14-5.07 (m, 1H), 3.75-3.65 (m, 1H), 3.64-3.51 (m, 2H), 3.45-3.24 (m, 6H), 3.25-3.19 (m, 1H), 3.18-3.10 (m, 1H), 2.63-2.47 (m, 2H), 2.15-2.03 (m, 2H), 2.00-1.70 (m, 5H), 1.11 (d, J=6.4 Hz, 6H).

### Example 23

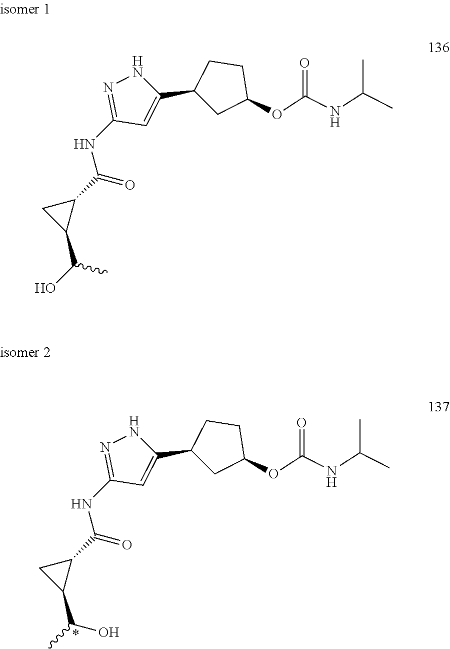

### Synthetic Route for Compounds 136 & 137 as Follows:

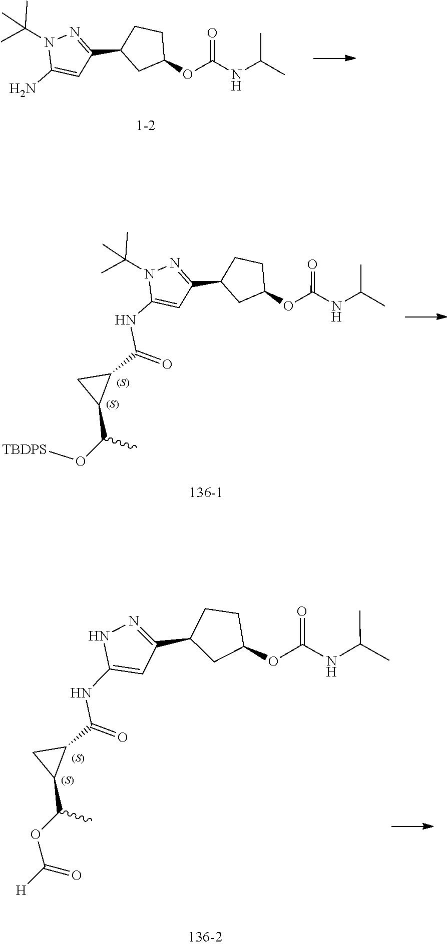

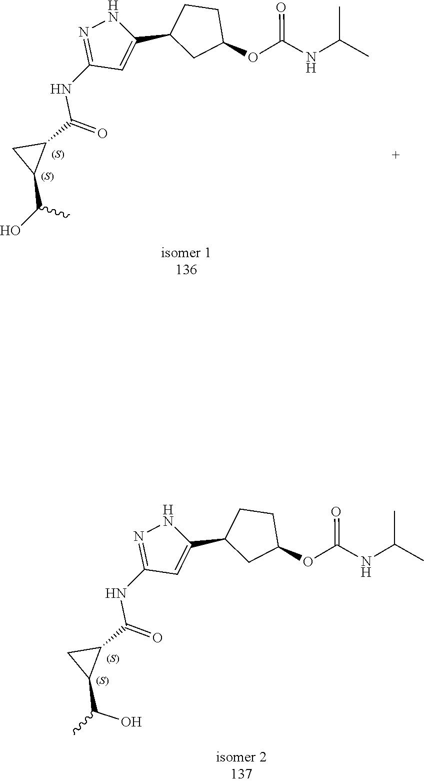

### Step 1 (Synthesis of Compound 136-1)

To a solution of O (620 mg, 1.7 mmol, 2.0 eq) in acetonitrile (10 mL) were added 1-2 (250 mg, 0.85 mmol, 1.0 eq), DIEA (330 mg. 2.5 mmol, 3.0 eq) and CMPI (434 mg, 1.7 mmol, 2.0 eq) at room temperature, then the mixture was heated to reflux and stirred for 12 hours. After completion of the reaction, the reaction mixture was cooled to room temperature and poured into water, and extracted with EtOAc (×3). The combined organic layers were washed with brine, dried over Na₂SO₄ and concentrated. The residue was purified by column chromatography on silica gel (PE:EtOAc=2:1) to give the 136-1 (160.0 mg). LCMS (ESI, m/z): [M+H]⁺=658.8.

### Step 2 (Synthesis of Compound 136-2)

The solution of 136-1 (360 mg, 0.55 mmol, 1.0 eq) in formic acid (5 mL) was heated to 85° C. and stirred for 12 hours. After completion of the reaction, the mixture was concentrated and the residue was purified by prep-TLC (EtOAc) to give 136-2 (190.0 mg). LCMS (ESI, m/z): [M+H]⁺=393.0.

### Step 3 (Synthesis of Compounds 136 & 137)

To a solution of 136-2 (190 mg, 0.48 mmol, 1.0 eq) in MeOH (2 mL) was added K₂CO₃ (66 mg, 0.48 mmol, 1.0 eq), the mixture was stirred at room temperature for 2 hours. After completion of the reaction, the reaction mixture was poured into water and the mixture was extracted with DCM:MeOH (10:1, ×3). The combined organic layers were washed with brine, dried over Na₂SO₄ and concentrated. The residue was purified by prep-TLC (DCM:MeOH=10:1), then Pre-HPLC (acetonitrile with 0.05% TFA in water, 20% to 40%) to give compound 136 (isomer 1, 19.7 mg) and compound 137 (isomer 2, 25 mg).

The relevant characterization data for compound 136 was as follows: LCMS (ESI, m/z): [M+H]⁺=365.4; ¹HNMR (400 MHz, METHANOL-d₄, ppm): δ 6.26 (s, 1H), 5.13-5.06 (m, 1H), 3.76-3.67 (m, 1H), 3.39-3.35 (m, 1H), 3.19-3.12 (m, 1H), 2.55-2.48 (m, 1H), 2.13-2.11 (m, 1H), 1.95-1.68 (m, 5H), 1.55-1.48 (m, 1H), 1.28-1.24 (m, 3H), 1.16-1.08 (m, 7H), 0.97-0.93 (m, 1H).

The relevant characterization data for compound 137 was as follows: LCMS (ESI, m/z): [M+H]⁺=365.4; ¹HNMR (400 MHz, METHANOL-d₄, ppm): δ 6.25 (s, 1H), 5.13-5.06 (m, 1H), 3.76-3.67 (m, 1H), 3.40-3.35 (m, 1H), 3.19-3.10 (m, 1H), 2.58-2.48 (m, 1H), 2.15-2.11 (m, 1H), 1.99-1.71 (m, 5H), 1.54-1.48 (m, 1H), 1.29-1.24 (m, 3H), 1.18-1.08 (m, 7H), 0.97-0.87 (m, 1H).

### Example 24

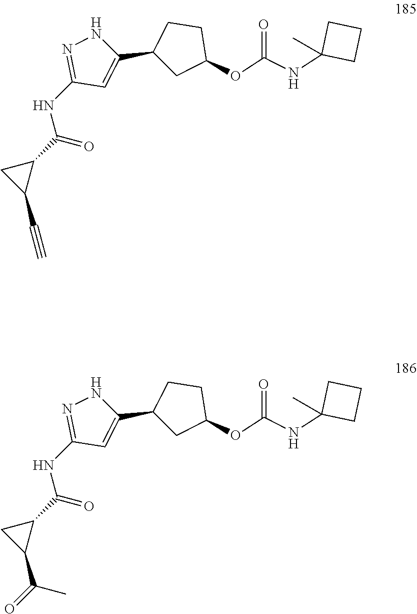 [SMILES: C#C[C@H]1C[C@@H]1C(=O)Nc1cc([C@H]2CC[C@@H](OC(=O)NC3(C)CCC3)C2)[nH]n1 ; CC(=O)[C@H]1C[C@@H]1C(=O)Nc1cc([C@H]2CC[C@@H](OC(=O)NC3(C)CCC3)C2)[nH]n1]

### Synthetic Route for Compounds 185 & 186 as Follows:

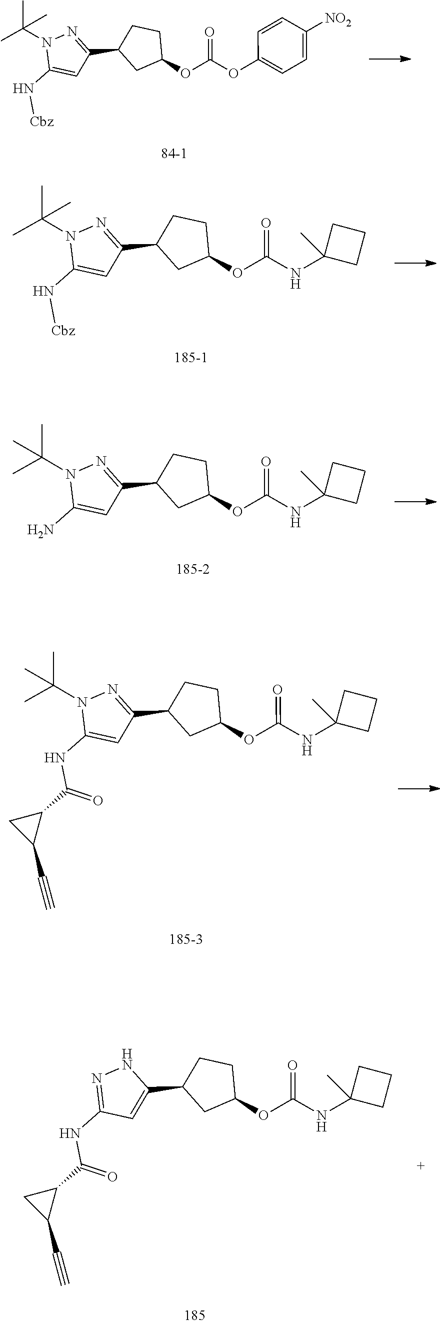 [SMILES: CC(C)(C)n1nc([C@H]2CC[C@@H](OC(=O)Oc3ccc([N+](=O)[O-])cc3)C2)cc1NC(=O)OCc1ccccc1 ; CC1(NC(=O)O[C@@H]2CC[C@H](c3cc(NC(=O)OCc4ccccc4)n(C(C)(C)C)n3)C2)CCC1 ; CC1(NC(=O)O[C@@H]2CC[C@H](c3cc(N)n(C(C)(C)C)n3)C2)CCC1 ; C#C[C@H]1C[C@@H]1C(=O)Nc1cc([C@H]2CC[C@@H](OC(=O)NC3(C)CCC3)C2)nn1C(C)(C)C ; C#C[C@H]1C[C@@H]1C(=O)Nc1cc([C@H]2CC[C@@H](OC(=O)NC3(C)CCC3)C2)[nH]n1]

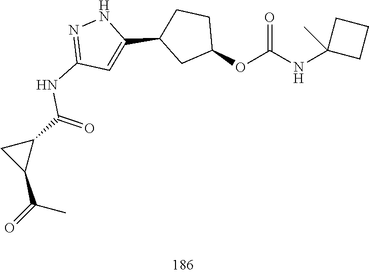 [SMILES: CC(=O)[C@H]1C[C@@H]1C(=O)Nc1cc([C@H]2CC[C@@H](OC(=O)NC3(C)CCC3)C2)[nH]n1]

### Step 1 (Synthesis of Compound 185-1)

To a solution of 84-1 (160 mg, 0.31 mmol, 1.0 eq) in DCM (5 mL) were added 1-methylcyclobutan-1-amine hydrochloride (76 mg, 0.62 mmol, 2.0 eq) and DIEA (240 mg, 1.86 mmol, 6.0 eq) at room temperature. Then the mixture was heated to 40° C. and stirred for 12 hours. After completion of the reaction, the reaction mixture was poured into water and the mixture was extracted with DCM (×3). The combined organic layers were washed with brine, dried over Na₂SO₄ and concentrated. The residue was purified by prep-TLC (PE/EtOAc=3:1) to give 185-1 (120 mg). LCMS (ESI, m/z): [M+H]⁺=468.9.

### Step 2 (Synthesis of Compound 185-2)

To a solution of 185-1 (120 mg, 0.26 mmol, 1.0 eq) in EtOH (2 mL) was added Pd/C (60 mg, 100% wt), the resulting mixture was stirred under 50 Psi of hydrogen pressure for 12 hours at room temperature. After completion of the reaction, the mixture was filtered through a Celite pad, and the filtrate was concentrated to give a residue which was purified by prep-TLC (PE/EtOAc=1:1) to give 185-2 (80 mg). LCMS (ESI, m/z): [M+H]⁺=335.0.

### Step 3 (Synthesis of Compound 185-3)

To a solution of 185-2 (100 mg, 0.30 mmol, 1.0 eq) in acetonitrile (5 mL) were added G (49.4 mg, 0.45 mmol, 1.5 eq), DIEA (154 mg, 1.20 mmol, 4.0 eq) and CMPI (255 mg, 1.00 mmol, 3.0 eq). The mixture was stirred at 80° C. for 12 hours before it was cooled and diluted with water. The aqueous phase was extracted with DCM (×3). The combined organic phase was washed with brine, dried over Na₂SO₄, filtered and concentrated to give a residue which was purified by silica gel chromatography (petroleum ether:ethyl acetate=1:1) to give 185-3 (85 mg). LCMS (ESI, m/z): [M+H]⁺=427.2.

### Step 4 (Synthesis of Compounds 185 & 186)

The solution of 185-3 (85 mg, 0.20 mmol, 1.0 eq) in anhydrous formic acid (5 mL) was stirred at 80° C. for 2 hours. HPLC showed the reaction was completed. Then the mixture was concentrated to give a residue, which was purified by prep-HPLC (acetonitrile with 0.1% FA in water 25% to 49%.) to give 185 (9.0 mg) and 186 (7.0 mg).

The relevant characterization data for compound 185 was as follows: LCMS (ESI, m/z): [M+H]⁺=371.2; H NMR (400 MHz, METHANOL-d₄, ppm): δ 6.31 (s, 1H), 5.13-4.99 (m, 1H), 3.21-3.09 (m, 1H), 2.56-2.43 (m, 1H), 2.35-2.20 (m, 3H), 2.15-2.04 (m, 2H), 1.94-1.74 (m, 9H), 1.42-1.33 (m, 4H), 1.19-1.11 (m, 1H).

The relevant characterization data for compound 186 was as follows: LCMS (ESI, m/z): [M+H]⁺=389.2; ¹H NMR (400 MHz, METHANOL-d₄, ppm): δ 6.32 (s, 1H), 5.13-4.99 (m, 1H), 3.19-3.11 (m, 1H), 2.57-2.43 (m, 2H), 2.38-2.19 (m, 6H), 2.16-2.04 (m, 1H), 1.99-1.72 (m, 8H), 1.50-1.44 (m, 1H), 1.42-1.34 (m, 4H).

The preparation of the intermediates in the table 1 below can be carried out by referring to the similar methods in the above-mentioned examples, the relevant characterization data was as follows:

TABLE 1

| | | LCMS | |
|---|---|---|---|
| Inter- | | (ESI, | |
| me- | | m/z): | |
| diate | Structure | [M + H]⁺ | Chiral purity and Analytical method |
| 26-1 | 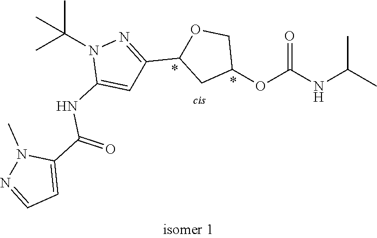 [SMILES: CC(C)NC(=O)OC1COC(c2cc(NC(=O)c3ccnn3C)n(C(C)(C)C)n2)C1] | 419.2 | Chiral purity 99.11% ee, retention time 1.864 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 26-2 | 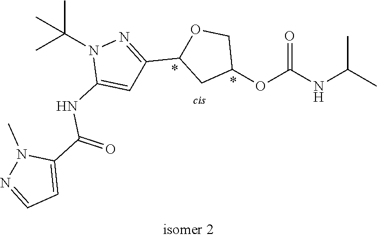 [SMILES: CC(C)NC(=O)OC1COC(c2cc(NC(=O)c3ccnn3C)n(C(C)(C)C)n2)C1] | 419.2 | Chiral purity 100% ee, retention time 3.266 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 50-1 | 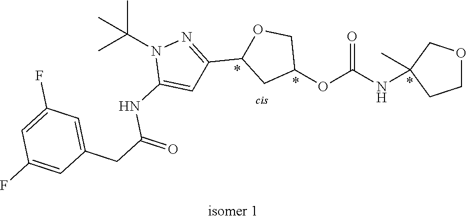 [SMILES: CC1(NC(=O)OC2COC(c3cc(NC(=O)Cc4cc(F)cc(F)c4)n(C(C)(C)C)n3)C2)CCOC1] | 507.2 | Chiral purity 95.11% de, retention time 3.668 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 50-2 | 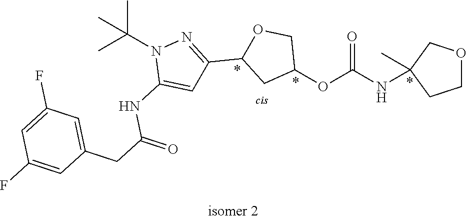 [SMILES: CC1(NC(=O)OC2COC(c3cc(NC(=O)Cc4cc(F)cc(F)c4)n(C(C)(C)C)n3)C2)CCOC1] | 507.2 | Chiral purity 92.56% de, retention time 3.819 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1 %DEA), flowing at 1.5 mL/min. |
| 50-3 | 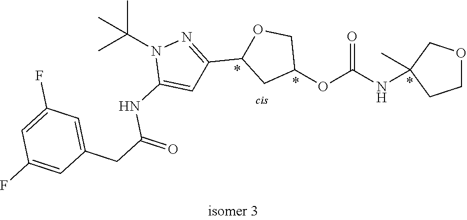 [SMILES: CC1(NC(=O)OC2COC(c3cc(NC(=O)Cc4cc(F)cc(F)c4)n(C(C)(C)C)n3)C2)CCOC1] | 507.2 | Chiral purity 100.0% de, retention time 9.797 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 50-4 | 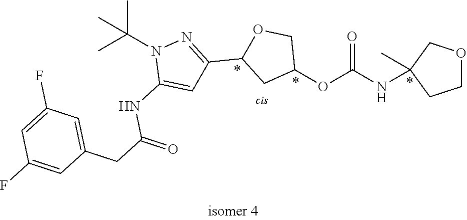 [SMILES: CC1(NC(=O)OC2COC(c3cc(NC(=O)Cc4cc(F)cc(F)c4)n(C(C)(C)C)n3)C2)CCOC1] | 507.2 | Chiral purity 100.0% de, retention time 11.229 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 62-1 | 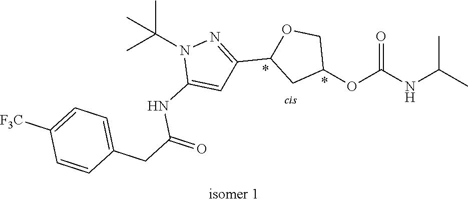 [SMILES: CC(C)NC(=O)OC1COC(c2cc(NC(=O)Cc3ccc(C(F)(F)F)cc3)n(C(C)(C)C)n2)C1] | 497.2 | Chiral purity 100% ee, retention time 3.172 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 62-2 | 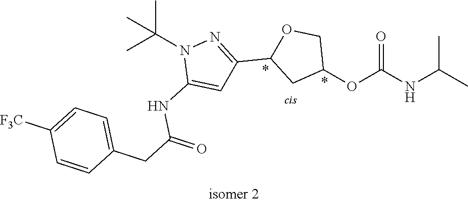 [SMILES: CC(C)NC(=O)OC1COC(c2cc(NC(=O)Cc3ccc(C(F)(F)F)cc3)n(C(C)(C)C)n2)C1] | 497.2 | Chiral purity 99.28% ee, retention time 3.956 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 64-1 | 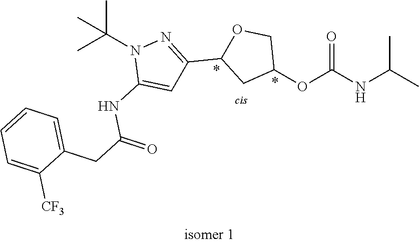 [SMILES: CC(C)NC(=O)OC1COC(c2cc(NC(=O)Cc3ccccc3C(F)(F)F)n(C(C)(C)C)n2)C1] | 497.2 | Chiral purity 100% ee, retention time 3.188 min. Chiral SFC analysis was performed Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 64-2 | 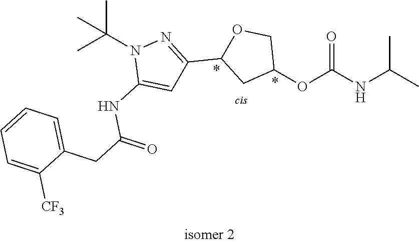 [SMILES: CC(C)NC(=O)OC1COC(c2cc(NC(=O)Cc3ccccc3C(F)(F)F)n(C(C)(C)C)n2)C1] | 497.2 | Chiral purity 99.40% ee, retention time 4.372 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 66-1 | 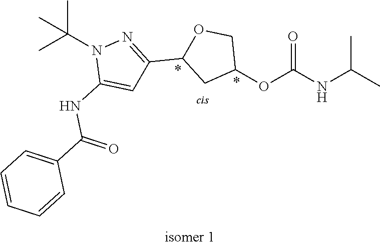 [SMILES: CC(C)NC(=O)OC1COC(c2cc(NC(=O)c3ccccc3)n(C(C)(C)C)n2)C1] | 415.2 | Chiral purity 100% ee, retention time 0.593 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 66-2 | 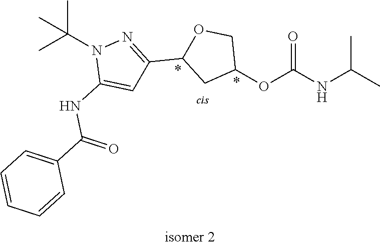 [SMILES: CC(C)NC(=O)OC1COC(c2cc(NC(=O)c3ccccc3)n(C(C)(C)C)n2)C1] | 415.2 | Chiral purity 99.22% ee, retention time 0.827 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and gradient of 5-40% MeOH (0.1% DEA), flowing at 1.5 mL/min. |
| 74-1 | 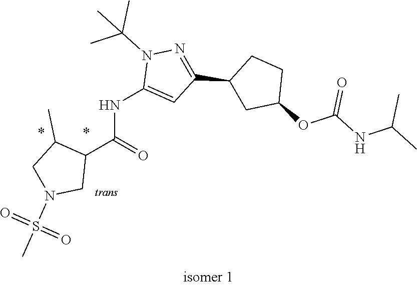 [SMILES: CC(C)NC(=O)O[C@@H]1CC[C@H](c2cc(NC(=O)C3CN(S(C)(=O)=O)CC3C)n(C(C)(C)C)n2)C1] | 498.2 | Chiral purity 99.90% de, retention time: 2.947 min, Chiral SFC analysis was performed on a Waters UPCC, DAICEL CHIRALPAK ®OD-3, 250* 4.6 mm 3 μm column heated to 35° C., eluted with a mobile phase of CO₂ and gradient of 20% MeOH (0.1% DEA), flowing at 2 mL/min. |
| 74-2 | 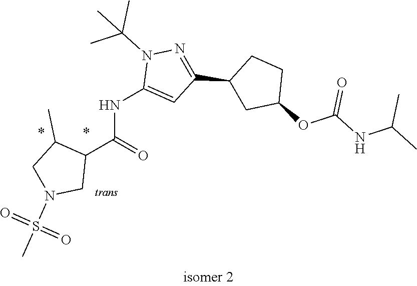 [SMILES: CC(C)NC(=O)O[C@@H]1CC[C@H](c2cc(NC(=O)C3CN(S(C)(=O)=O)CC3C)n(C(C)(C)C)n2)C1] | 498.2 | Chiral purity 98.08% de, retention time: 3.583 min. Chiral SFC analysis was performed on a Waters UPCC (CA-352), DAICEL CHIRALPAK ®OD-3, 250* 4.6 mm 3 μm column heated to 35° C., eluted with a mobile phase of CO₂ and gradient of 20% MeOH (0.1% DEA), flowing at 2 mL/min. |
| 113-1 | 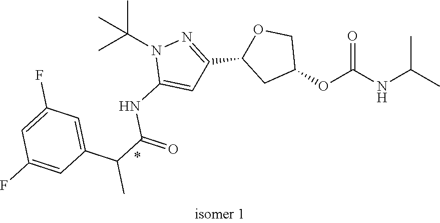 [SMILES: CC(C)NC(=O)O[C@H]1CO[C@@H](c2cc(NC(=O)C(C)c3cc(F)cc(F)c3)n(C(C)(C)C)n2)C1] | 479.4 | Chiral purity 100% de, retention time: 1.986 min. Chiral SFC analysis was performed on a Waters UPCC (CA-060), column: DAICEL CHIRALPAK ®OJ, 100*3.0 mm 3 μm, column heated to 35° C., eluted with a mobile phase of CO₂ and gradient of 5-40% MeOH (0.1% DEA), flowing at 1.5 mL/min. |
| 113-2 | 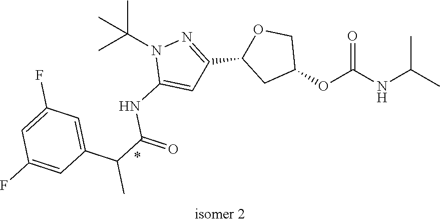 [SMILES: CC(C)NC(=O)O[C@H]1CO[C@@H](c2cc(NC(=O)C(C)c3cc(F)cc(F)c3)n(C(C)(C)C)n2)C1] | 479.4 | Chiral purity 100% de, retention time: 2.305 min. Chiral SFC analysis was performed on a Waters UPCC (CA-060), column: DAICEL CHIRALPAK ®OJ, 100*3.0 mm 3 μm, column heated to 35° C., eluted with a mobile phase of CO₂ and gradient of 5-40% MeOH (0.1% DEA), flowing at 1.5 mL/min. |
| 150-1 | 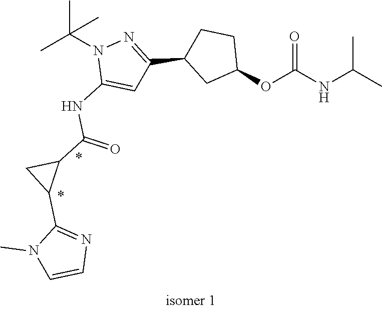 [SMILES: CC(C)NC(=O)O[C@@H]1CC[C@H](c2cc(NC(=O)C3CC3c3nccn3C)n(C(C)(C)C)n2)C1] | 456.8 | Chiral purity 99.82% de, retention time: 3.314 min. Chiral SFC analysis was performed on a Waters UPCC, IG-3 column heated to 35° C., eluted with a mobile phase of CO₂ and gradient of 30% MeOH (0.1% DEA), flowing at 2 mL/min. |
| 150-2 | 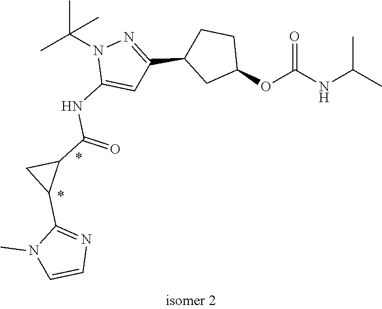 [SMILES: CC(C)NC(=O)O[C@@H]1CC[C@H](c2cc(NC(=O)C3CC3c3nccn3C)n(C(C)(C)C)n2)C1] | 456.8 | Chiral purity 99.28% de, retention time: 4.134 min. Chiral SFC analysis was performed on a Waters UPCC, IG-3 column heated to 35° C., eluted with a mobile phase of CO₂ and gradient of 30% MeOH (0.1% DEA), flowing at 2 mL/min. |
| 173-1 | 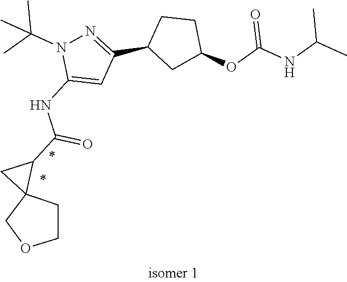 [SMILES: CC(C)NC(=O)O[C@@H]1CC[C@H](c2cc(NC(=O)C3CC34CCOC4)n(C(C)(C)C)n2)C1] | 433.0 | Chiral purity 97.1% de, retention time: 1.505 min. Chiral SFC analysis was performed on a Waters UPCC (CA-060), DAICELCHIRALCEL ® OJ, 100x3.0 mm, 3 μm, column heated to 35° C., eluted with a mobile phase of CO₂ and gradient of 5-40% EtOH (0.1% DEA), flowing at 1.5 mL/min. |
| 173-2 | 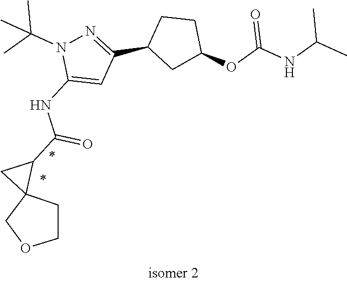 [SMILES: CC(C)NC(=O)O[C@@H]1CC[C@H](c2cc(NC(=O)C3CC34CCOC4)n(C(C)(C)C)n2)C1] | 433.0 | Chiral purity 100% de, retention time: 1.894 min. Chiral SFC analysis was performed on a Waters UPCC (CA-060), DAICELCHIRALCEL ® OJ, 100x3.0 mm, 3 μm, column heated to 35° C., eluted with a mobile phase of CO₂ and gradient of 5-40% EtOH (0.1% DEA), flowing at 1.5 mL/min. |
| 173-3 | 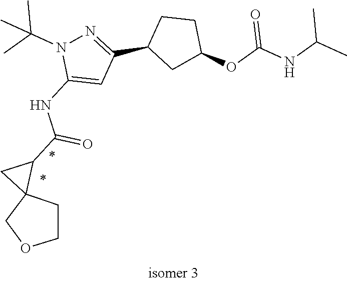 [SMILES: CC(C)NC(=O)O[C@@H]1CC[C@H](c2cc(NC(=O)C3CC34CCOC4)n(C(C)(C)C)n2)C1] | 433.0 | Chiral purity 97.9% de, retention time: 3.275 min. Chiral SFC analysis was performed on a Waters UPCC (CA-060), DAICELCHIRALCEL ® OZ, 100x3.0 mm, 3 μm, column heated to 35° C., eluted with a mobile phase of CO₂ and gradient of 5-40% EtOH (0.1% DEA), flowing at 1.5 mL/min. |
| 173-4 | 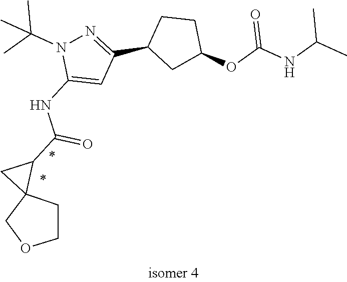 [SMILES: CC(C)NC(=O)O[C@@H]1CC[C@H](c2cc(NC(=O)C3CC34CCOC4)n(C(C)(C)C)n2)C1] | 433.0 | Chiral purity 97.8% de, retention time:3.581 min. Chiral SFC analysis was performed on a Waters UPCC (CA-060), DAICELCHIRALCEL ® OZ, 100x3.0 mm, 3 μm, column heated to 35° C., eluted with a mobile phase of CO₂ and gradient of 5-40% EtOH (0.1% DEA), flowing at 1.5 mL/min. |

The preparation of the compounds in the table 2 below can be carried out by referring to the similar methods in the above-mentioned examples, the relevant characterization data was as follows:

TABLE 2

| | | LCMS | |
|---|---|---|---|
| | | (ESI, | |
| | | m/z): | |
| Com- | | [M + | ¹HNMR, |
| pound | Structrue | H]⁺ | Chiral purity and Analytical method |
| 2 | 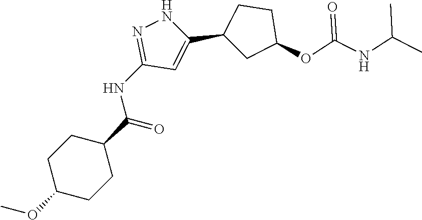 [SMILES: CO[C@H]1CC[C@H](C(=O)Nc2cc([C@H]3CC[C@@H](OC(=O)NC(C)C)C3)[nH]n2)CC1] | 393.1 | ¹HNMR (400 MHz, method-d₄, ppm): δ 6.33 (s, 1H), 5.10-5.07 (m, 1H), 3.71-3.68 (m, 1H), 3.36 (s, 3H), 3.22-3.13 (m, 2H), 2.53-2.46 (m, 1H), 2.38-2.30 (m, 1H), 2.18-2.06 (m, 3H), 1.95-1.76 (m, 6H), 1.64-1.53 (m, 2H), 1.29-1.18 (m, 2H), 1.12-1.10 (m, 6H). |
| 6 | 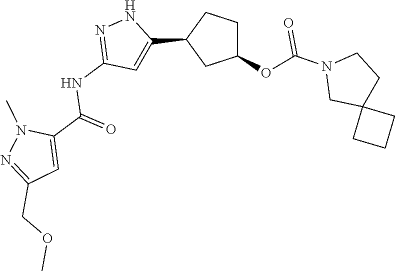 [SMILES: COCc1cc(C(=O)Nc2cc([C@H]3CC[C@@H](OC(=O)N4CCC5(CCC5)C4)C3)[nH]n2)n(C)n1] | 457.3 | ¹H NMR (400 MHz, METHANOL-d₄, ppm): δ 6.91 (s, 1H), 6.55-6.45 (m, 1H), 5.13-5.09 (m, 1H), 4.43 (s, 2H), 4.12 (s, 3H), 3.37 (s, 3H), 3.36-3.18 (m, 4H), 2.55-2.39 (m, 1H), 2.19-2.05 (m, 1H), 2.01-1.78 (m, 13H). |
| 7 | 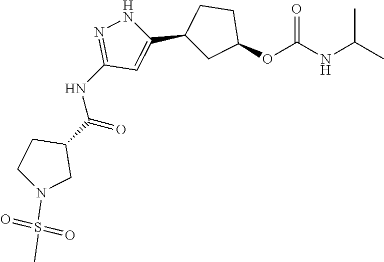 [SMILES: CC(C)NC(=O)O[C@@H]1CC[C@H](c2cc(NC(=O)[C@H]3CCN(S(C)(=O)=O)C3)n[nH]2)C1] | 428.0 | ¹HNMR (400 MHz, METHANOL-d₄, ppm): δ 6.35 (s, 1H), 5.09-5.07 (m, 1H), 3.75-3.64 (m, 1H), 3.62-3.50 (m, 2H), 3.50-3.37 (m, 2H), 3.28-3.21 (m, 1H), 3.20-3.10 (m, 1H), 2.92 (s, 3H), 2.56-2.43 (m, 1H), 2.30-2.17 (m, 2H), 2.11-2.03 (m, 1H), 1.96-1.71 (m, 4H), 1.11 (d, J = 6.4 Hz, 6H). |
| 8 | 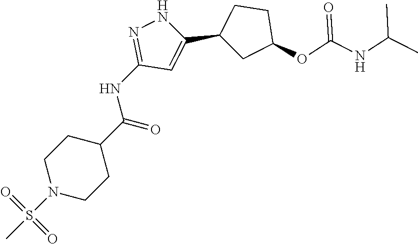 [SMILES: CC(C)NC(=O)O[C@@H]1CC[C@H](c2cc(NC(=O)C3CCN(S(C)(=O)=O)CC3)n[nH]2)C1] | 442.1 | ¹HNMR (400 MHz, METHANOL-d₄, ppm): δ 6.34 (s, 1H), 5.15-5.05 (m, 1H), 3.83-3.80 (m, 2H), 3.75-3.66 (m, 1H), 3.20-3.10 (m, 1H), 2.85-2.77 (m, 5H), 2.54-2.45 (m, 2H), 2.15-2.07 (m, 1H), 1.97-1.72 (m, 8H), 1.12-1.09 (m, 6H). |
| 12 | 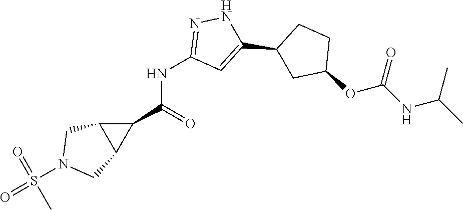 [SMILES: CC(C)NC(=O)O[C@@H]1CC[C@H](c2cc(NC(=O)[C@H]3[C@@H]4CN(S(C)(=O)=O)C[C@@H]43)n[nH]2)C1] | 440.2 | ¹HNMR (400 MHz, DMSO-d₆, ppm): δ 12.01 (brs, 1H), 10.50 (brs, 1H), 6.95-6.92 (m, 1H), 6.26 (s, 1H), 5.03-4.95 (m, 1H), 3.62-3.50 (m, 1H), 3.47-3.37 (m, 4H), 3.14-2.96 (m, 1H), 2.92 (s, 3H), 2.46-2.41 (m, 1H), 2.10-1.84 (m, 5H), 1.80-1.50 (m, 3H), 1.02 (d, J = 6.4 Hz, 6H). |
| 13 | 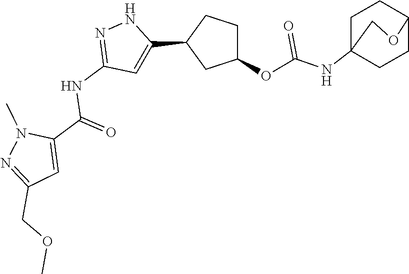 [SMILES: COCc1cc(C(=O)Nc2cc([C@H]3CC[C@@H](OC(=O)NC45CCC(CC4)OC5)C3)[nH]n2)n(C)n1] | 473.2 | ¹HNMR (400 MHz, DMSO-d₆, ppm): δ 12.22 (brs, 1H), 10.72 (brs, 1H), 7.11 (s, 1H), 6.89 (brs, 1H), 6.41 (s, 1H), 5.02-4.98 (m, 1H), 4.34 (s, 2H), 4.05 (s, 3H), 3.73 (s, 2H), 3.64-3.62 (s, 1H), 3.37 (s, 3H), 3.09-3.03 (m, 1H), 2.03-1.97 (m, 1H), 1.92-1.88 (m, 5H), 1.86-1.64 (m, 8H). |
| 14 | 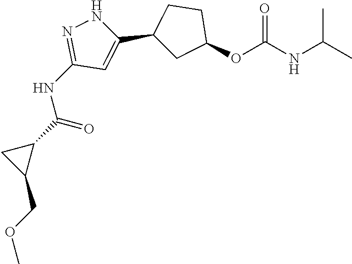 [SMILES: COC[C@H]1C[C@@H]1C(=O)Nc1cc([C@H]2CC[C@@H](OC(=O)NC(C)C)C2)[nH]n1] | 365.2 | ¹HNMR (400 MHz, DMSO-d₆, ppm): δ 11.99 (brs, 1H), 10.49 (brs, 1H), 6.94-6.92 (m, 1H), 6.27 (s, 1H), 4.99-4.97 (m, 1H), 3.57-3.54 (m, 1H), 3.37-3.36 (m, 1H), 3.23 (s, 3H), 3.18-3.15 (m, 1H), 3.14-3.12 (m, 1H), 2.50-2.49 (m, 1H), 1.99-1.42 (m, 7H), 1.02 (d, J = 6.4 Hz, 6H), 0.95-0.93 (m, 1H), 0.73-0.72 (m, 1H). |
| 25 | 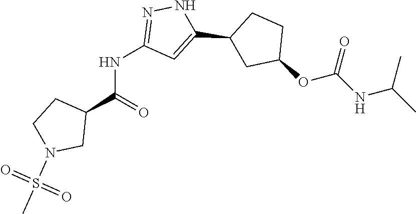 [SMILES: CC(C)NC(=O)O[C@@H]1CC[C@H](c2cc(NC(=O)[C@@H]3CCN(S(C)(=O)=O)C3)n[nH]2)C1] | 428.2 | ¹HNMR (400 MHz, METHANOL-d₄, ppm): δ 6.35 (s, 1H), 5.09-5.08 (m, 1H), 3.70-3.66 (m, 1H), 3.62-3.37 (m, 4H), 3.38-3.21 (m, 1H), 3.17-3.13 (m, 1H), 2.93 (s, 3H), 2.54-2.48 (m, 1H), 2.32-2.25 (m, 2H), 2.11-2.06 (m, 1H), 1.96-1.76 (m, 4H), 1.11 (d, J = 6.4 Hz, 6H). |
| 26 | 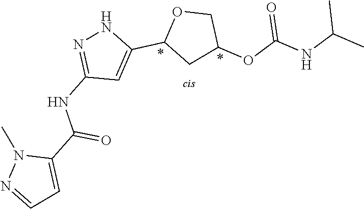 [SMILES: CC(C)NC(=O)OC1COC(c2cc(NC(=O)c3ccnn3C)n[nH]2)C1] | 363.3 | ¹H NMR (400 MHz, METHANOL-d₄, ppm): δ 7.53 (d, J = 2.4 Hz, 1H), 6.97 (d, J = 2.4 Hz, 1H), 6.53 (s, 1H), 5.26 (s, 1H), 5.09-5.01 (m, 1H), 4.18 (s, 3H), 4.08-4.06 (m, 1H), 4.01-3.95 (m, 1H), 3.73-3.69 (m, 1H), 2.78- 2.71 (m, 1H), 2.18-2.16 (m, 1H), 1.13-1.07 (m, 6H). |
| | It's obtained from the | | |
| | intermediate 26-1. | | |
| 27 | 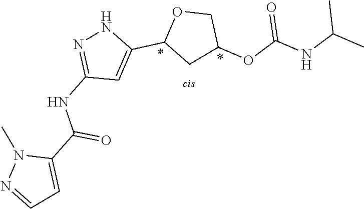 [SMILES: CC(C)NC(=O)OC1COC(c2cc(NC(=O)c3ccnn3C)n[nH]2)C1] | 363.3 | ¹H NMR (400 MHz, METHANOL-d₄, ppm): δ 7.55 (d, J = 2.0 Hz, 1H), 6.98 (d, J = 2.0 Hz, 1H), 6.55 (s, 1H), 5.27 (s, 1H), 5.10-5.03 (m, 1H), 4.18 (s, 3H), 4.08-4.06 (m, 1H), 4.02-3.96 (m, 1H), 3.75-3.67 (m, 1H), 2.80- 2.71 (m, 1H), 2.24-2.13 (m, 1H), 1.20- 1.09 (m, 6H). |
| | It's obtained from the | | |
| | intermediate 26-2. | | |
| 28 | 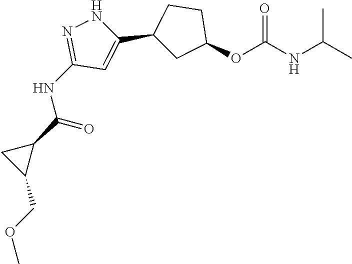 [SMILES: COC[C@@H]1C[C@H]1C(=O)Nc1cc([C@H]2CC[C@@H](OC(=O)NC(C)C)C2)[nH]n1] | 365.2 | ¹HNMR (400 MHz, DMSO-d₆, ppm): δ 12.00 (s, 1H), 10.49 (s, 1H), 6.94-6.90 (m, 1H), 6.25 (s, 1H), 5.04-4.95 (m, 1H), 3.65-3.52 (m, 1H), 3.40-3.33 (m, 1H), 3.23 (s, 3H), 3.10-2.98 (m, 2H), 2.50-2.40 (m, 1H), 2.00-1.35 (m, 7H), 1.02 (d, J = 6.6 Hz, 6H), 0.99-0.87 (m, 1H), 0.74-0.68 (m, 1H). |
| 32 | 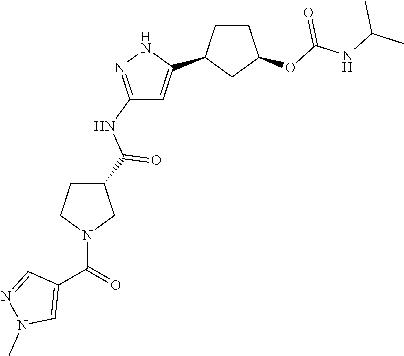 [SMILES: CC(C)NC(=O)O[C@@H]1CC[C@H](c2cc(NC(=O)[C@H]3CCN(C(=O)c4cnn(C)c4)C3)n[nH]2)C1] | 458.2 | ¹HNMR (400 MHz, DMSO-d₆, ppm): δ 12.07 (brs, 1H), 10.46 (d, J = 5.6 Hz, 1H), 8.15-8.13 (m, 1H), 7.75 (s, 1H), 6.95-6.89 (m, 1H), 6.31 (s, 1H), 5.04-4.98 (m, 1H), 3.88 (s, 3H), 3.74-3.50 (m, 4H), 3.34-3.13 (m, 2H), 3.09-3.03 (m, 1H), 2.23-2.10 (m, 2H), 2.07-1.85 (m, 3H), 1.80-1.53 (m, 3H), 1.02 (d, J = 6.4 Hz, 6H). |
| 33 | 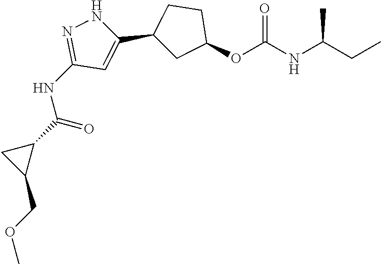 [SMILES: CC[C@H](C)NC(=O)O[C@@H]1CC[C@H](c2cc(NC(=O)[C@H]3C[C@@H]3COC)n[nH]2)C1] | 379.2 | ¹HNMR (400 MHz, METHANOL-d₄, ppm): δ 6.31 (s, 1H), 5.13-5.06 (m, 1H), 3.52-3.45 (m, 2H), 3.35 (s, 3H), 3.29-3.25 (m, 1H), 3.19-3.12 (m, 1H), 2.53-2.45 (m, 1H), 2.13-2.07 (m, 1H), 1.96-1.60 (m, 6H), 1.47-1.40 (m, 2H), 1.19-1.15 (m, 1H), 1.10-1.03 (m, 3H), 0.93-0.84 (m, 4H). |
| 34 | 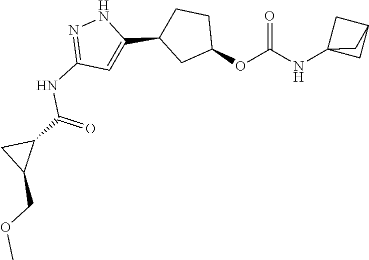 [SMILES: COC[C@H]1C[C@@H]1C(=O)Nc1cc([C@H]2CC[C@@H](OC(=O)NC34CC(C3)C4)C2)[nH]n1] | 389.1 | ¹HNMR (400 MHz, METHANOL-d₄, ppm): δ 6.30 (s, 1H), 5.12-5.05 (m, 1H), 3.49-3.45 (m, 1H), 3.35 (s, 3H), 3.29-3.23 (m, 1H), 3.19-3.09 (m, 1H), 2.57-2.46 (m, 1H), 2.36 (s, 1H), 2.12-2.09 (m, 1H), 1.99 (s, 6H), 1.93-1.72 (m, 5H), 1.67-1.62 (m, 1H), 1.20-1.15 (m, 1H), 0.89-0.84 (m, 1H). |
| 35 | 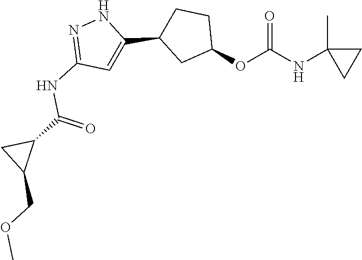 [SMILES: COC[C@H]1C[C@@H]1C(=O)Nc1cc([C@H]2CC[C@@H](OC(=O)NC3(C)CC3)C2)[nH]n1] | 377.1 | ¹HNMR (400 MHz, DMSO-d₆, ppm): δ 11.99 (brs, 1H), 10.49 (s, 1H), 7.34 (s, 1H), 6.25 (s, 1H), 5.08-4.95 (m, 1H), 3.40-3.35 (m, 1H), 3.23 (s, 3H), 3.18-3.13 (m, 1H), 3.11-2.95 (m, 1H), 2.46-2.41 (m, 1H), 2.02-1.96 (m, 1H), 1.89-1.80 (m, 1H), 1.77-1.38 (m, 5H), 1.22 (s, 3H), 0.96-0.92 (m, 1H), 0.75-0.70 (m, 1H), 0.60-0.54 (m, 2H), 0.47-0.43 (m, 2H). |
| 36 | 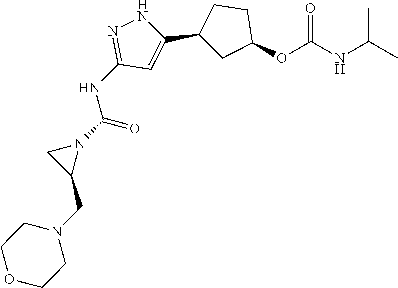 | 420.1 | ¹HNMR (400 MHz, DMSO-d₆, ppm): δ 10.51 (s, 1H), 8.15 (s, 1.52H, FA), 6.93 (d, J = 7.2 Hz, 1H), 6.26 (s, 1H), 5.04-4.96 (m, 1H), 3.63-3.56 (m, 6H), 3.09-3.01 (m, 1H), 2.47-2.35 (m, 6H), 2.23-2.17 (m, 1H), 2.02-1.93 (m, 1H), 1.89-1.80 (m, 1H), 1.76-1.52 (m, 4H), 1.28-1.20 (m, 1H), 1.03-0.93 (m, 7H), 0.68-0.60 (m, 1H). |
| 37 | 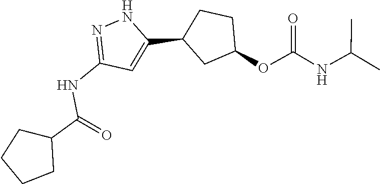 [SMILES: CC(C)NC(=O)O[C@@H]1CC[C@H](c2cc(NC(=O)C3CCCC3)n[nH]2)C1] | 349.1 | ¹HNMR (400 MHz, METHANOL-d₄, ppm): δ 6.36 (s, 1H), 5.14-5.05 (m, 1H), 3.75-3.62 (m, 1H), 3.20-3.10 (m, 1H), 2.85-2.77 (m, 1H), 2.53-2.46 (m, 1H), 2.14-1.62 (m, 13H), 1.11 (d, J = 6.4 Hz, 6H). |
| 38 | 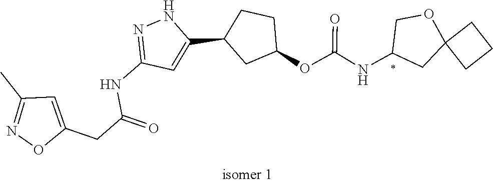 [SMILES: Cc1cc(CC(=O)Nc2cc([C@H]3CC[C@@H](OC(=O)NC4COC5(CCC5)C4)C3)[nH]n2)on1] | 444.0 | ¹H NMR (400 MHz, METHANOL-d₄, ppm): δ 6.38 (s, 1H), 6.24 (s, 1H), 5.17-5.08 (m, 1H), 4.23-4.15 (m, 1H), 3.97-3.90 (m, 1H), 3.88 (s, 2H), 3.58-3.45 (m, 1H), 3.23-3.10 (m, 1H), 2.56-2.40 (m, 1H), 2.37-1.50 (m, 16H). Chiral purity 99.12% de, retention time 5.575 min. Chiral SFC analysis was performed on a OJ-3, column heated to 35° C., eluted with a mobile phase of CO₂ and 21% EtOH, flowing at 2.0 |
| | | | mL/min. |
| 39 | 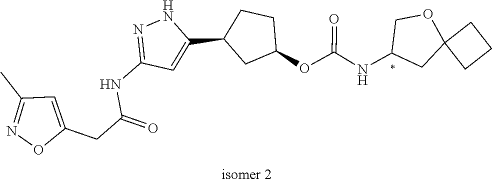 [SMILES: Cc1cc(CC(=O)Nc2cc([C@H]3CC[C@@H](OC(=O)NC4COC5(CCC5)C4)C3)[nH]n2)on1] | 444.1 | ¹H NMR (400 MHz, METHANOL-d₄, ppm): δ 6.38 (s, 1H), 6.24 (s, 1H), 5.17-5.08 (m, 1H), 4.20-4.13 (m, 1H), 3.97-3.90 (m, 1H), 3.89 (s, 2H), 3.59-3.51 (m, 1H), 3.23-3.10 (m, 1H), 2.58-2.50 (m, 1H), 2.35-1.50 (m, 16H). Chiral purity 95.74% de, retention time 7.562 min. Chiral SFC analysis was performed on a OJ-3, column heated to 35° C., eluted with a mobile phase of CO₂ and 21% EtOH, flowing at 2.0 mL/min. |
| 41 | 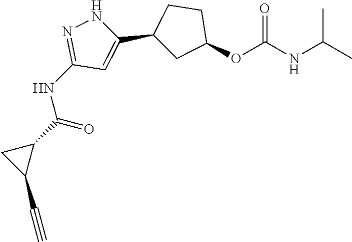 [SMILES: C#C[C@H]1C[C@@H]1C(=O)Nc1cc([C@H]2CC[C@@H](OC(=O)NC(C)C)C2)[nH]n1] | 345.0 | ¹HNMR (400 MHz, METHANOL-d₄, ppm): δ 6.31 (s, 1H), 5.12-5.06 (m, 1H), 3.73-3.67 (m, 1H), 3.18-3.10 (m, 1H), 2.56-2.45 (m, 1H), 2.24 (d, J = 2.0 Hz, 1H), 2.14-2.07 (m, 2H), 1.96-1.74 (m, 5H), 1.37-1.29 (m, 1H), 1.19-1.07 (m, 7H). |
| 42 | 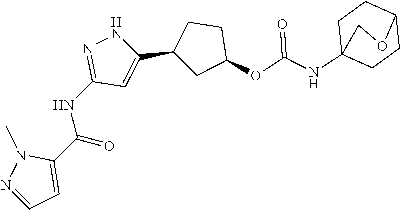 [SMILES: Cn1nccc1C(=O)Nc1cc([C@H]2CC[C@@H](OC(=O)NC34CCC(CC3)OC4)C2)[nH]n1] | 429.00 | ¹HNMR (300 MHz, METHANOL-d₄, ppm): δ 7.53 (d, J = 2.1 Hz, 1H), 6.97 (d, J = 2.1 Hz, 1H), 6.41 (s, 1H), 5.12-5.03 (m, 1H), 4.17 (s, 3H), 3.91 (s, 2H), 3.72 (s, 1H), 3.23-3.18 (m, 1H), 2.56-2.48 (m, 1H), 2.18-1.70 (m, 13H). |
| 43 | 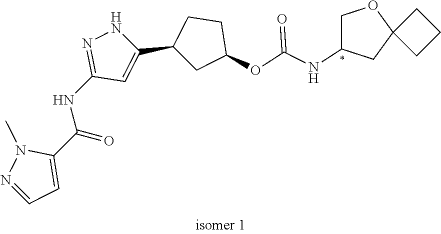 [SMILES: Cn1nccc1C(=O)Nc1cc([C@H]2CC[C@@H](OC(=O)NC3COC4(CCC4)C3)C2)[nH]n1] | 429.1 | ¹HNMR (300 MHz, METHANOL-d₄, ppm): δ 7.50 (d, J = 2.4 Hz, 1H), 6.94 (d, J = 2.1 Hz, 1H), 6.45 (s, 1H), 5.17-5.10 (m, 1H), 4.16-4.10 (m, 4H), 3.99-3.91 (m, 1H), 3.57-3.52 (m, 1H), 3.30-3.29 (m, 1H), 2.52-2.40 (m, 1H), 2.33-1.42 (m, 13H). Chiral purity 98.3% de, retention time 4.880 min. Chiral SFC analysis was performed on a Chiral-MIC, column heated to 35° C., eluted with a mobile phase of CO₂ and 21% EtOH, flowing at 2.0 mL/min. |
| 44 | 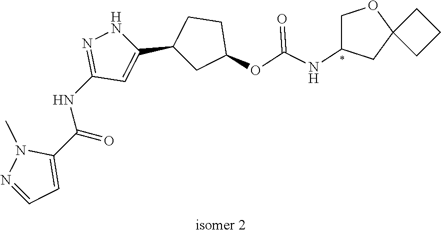 [SMILES: Cn1nccc1C(=O)Nc1cc([C@H]2CC[C@@H](OC(=O)NC3COC4(CCC4)C3)C2)[nH]n1] | 429.1 | ¹HNMR (300 MHz, METHANOL-d₄, ppm): δ 7.50 (d, J = 2.1 Hz, 1H), 6.94 (d, J = 2.1 Hz, 1H), 6.45 (s, 1H), 5.20-5.11 (m, 1H), 4.16-4.14 (m, 4H), 3.98-3.91 (m, 1H), 3.58-3.52 (m, 1H), 3.30-3.29 (m, 1H), 2.52-2.40 (m, 1H), 2.33-1.42 (m, 13H). Chiral purity 98.22% de, retention time 5.637 min. Chiral SFC analysis was performed on a Chiral-MIC, column heated to 35° C., eluted with a mobile phase of CO₂ and 21% EtOH, flowing at 2.0 mL/min. |
| 45 | 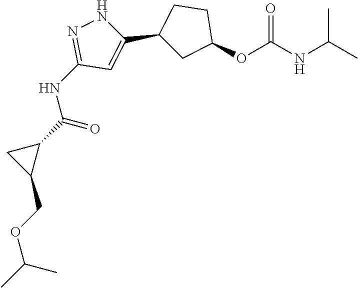 [SMILES: CC(C)NC(=O)O[C@@H]1CC[C@H](c2cc(NC(=O)[C@H]3C[C@@H]3COC(C)C)n[nH]2)C1] | 393.1 | ¹HNMR (400 MHz, DMSO-d₆, ppm) δ 12.00 (brs, 1H), 10.49 (s, 1H), 6.97-6.94 (s, 1H), 6.25 (s, 1H), 5.02-4.97 (s, 1H), 3.62-3.50 (m, 2H), 3.40-3.32 (m, 1H), 3.22-3.17 (m, 1H), 3.09-2.98 (m, 1H), 2.46-2.44 (m, 1H), 1.98-1.80 (m, 2H), 1.78-1.53 (m, 4H), 1.41-1.38 (m, 1H), 1.07 (d, J = 6.0 Hz, 6H), 1.02 (d, J = 6.4 Hz, 6H), 0.94-0.90 (m, 1H), 0.74-0.70 (m, 1H). |
| 46 | 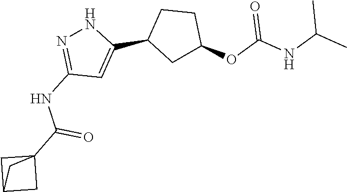 [SMILES: CC(C)NC(=O)O[C@@H]1CC[C@H](c2cc(NC(=O)C34CC(C3)C4)n[nH]2)C1] | 347.1 | ¹HNMR (400 MHz, METHANOL-d₄, ppm): δ 6.30 (s, 1H), 5.14-5.06 (s, 1H), 3.75-3.67 (m, 1H), 3.19-3.10 (m, 1H), 2.53-2.44 (m, 2H), 2.14 (s, 6H), 2.14-2.08 (m, 1H), 1.98-1.70 (m, 4H), 1.13-1.09 (m, 6H). |
| 47 | 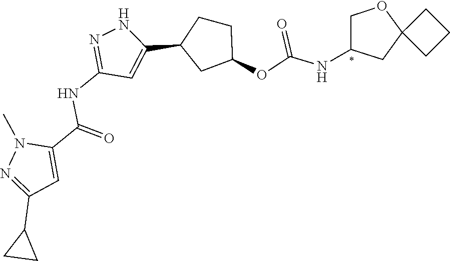 [SMILES: Cn1nc(C2CC2)cc1C(=O)Nc1cc([C@H]2CC[C@@H](OC(=O)NC3COC4(CCC4)C3)C2)[nH]n1] | 469.1 | ¹HNMR (400 MHz, METHANOL-d₄, ppm): δ 6.61 (s, 1H), 6.45 (s, 1H), 5.17-5.10 (m, 1H), 4.20-4.12 (m, 1H), 4.06 (s, 3H), 3.99-3.89 (m, 1H), 3.58-3.54 (m, 1H), 3.25-3.16 (m, 1H), 2.55-2.48 (m, 1H), 2.35-2.10 (m, 5H), 2.03-1.50 (m, 9H), 0.96-0.91 (m, 2H), 0.74-0.70 (m, 2H). Chiral purity 100% de, retention time 7.297 min. Chiral SFC analysis was performed on a Waters UPCC, OJ-3, 4.6 mm*250 mm, 3 μm, column heated to 35° C., eluted with a mobile phase of CO₂ and 15% EtOH, flowing at 2.0 mL/min. |
| 48 | 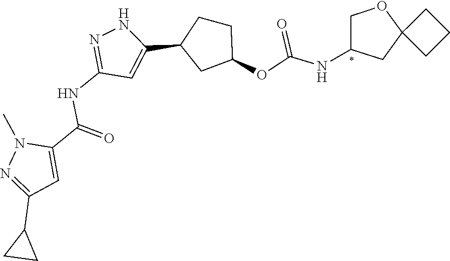 [SMILES: Cn1nc(C2CC2)cc1C(=O)Nc1cc([C@H]2CC[C@@H](OC(=O)NC3COC4(CCC4)C3)C2)[nH]n1] | 469.1 | ¹HNMR (400 MHz, METHANOL-d₄, ppm): δ 6.61 (s, 1H), 6.45 (s, 1H), 5.16-5.10 (m, 1H), 4.20-4.15 (m, 1H), 4.06 (s, 3H), 3.98-3.89 (m, 1H), 3.59-3.54 (m, 1H), 3.23-3.17 (m, 1H), 2.58-2.52 (m, 1H), 2.29-2.03 (m, 5H), 2.02-1.50 (m, 9H), 0.96-0.91 (m, 2H), 0.75-0.70 (m, 2H). Chiral purity 98.84% de, retention time 8.638 min. Chiral SFC analysis was performed on a Waters UPCC, OJ-3, 4.6 mm*250 mm, 3 μm, column heated to 35° C., eluted with a mobile phase of CO₂ and 15% EtOH, flowing at 2.0 mL/min. |
| 49 | 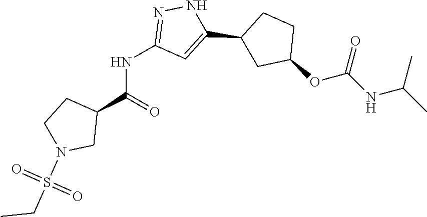 [SMILES: CCS(=O)(=O)N1CC[C@@H](C(=O)Nc2cc([C@H]3CC[C@@H](OC(=O)NC(C)C)C3)[nH]n2)C1] | 442.0 | ¹HNMR (400 MHz, METHANOL-d₄, ppm): δ 6.34 (s, 1H), 5.12-5.06 (m, 1H), 3.73-3.60 (m, 2H), 3.59-3.40 (m, 3H), 3.30-3.22 (m, 1H) 3.17-3.10 (m, 3H), 2.56-2.45 (m, 1H), 2.29-2.17 (m, 2H) 2.11-2.09 (m, 1H) 1.96-1.72 (m, 4H) 1.37-1.29 (m, 3H) 1.11 (d, J = 6.4 Hz, 6H). |
| 50 | 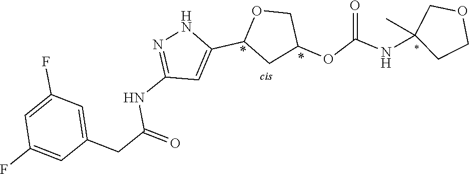 [SMILES: CC1(NC(=O)OC2COC(c3cc(NC(=O)Cc4cc(F)cc(F)c4)n[nH]3)C2)CCOC1] | 451.4 | ¹HNMR (400 MHz, METHANOL-d₄, ppm): δ 7.00-6.98 (m, 2H), 6.90-6.85 (m, 1H), 6.44 (s, 1H), 5.37-5.22 (m, 1H), 5.05-5.03 (m, 1H), 4.07-4.04 (m, 1H), 3.98-3.88 (m, 2H), 3.86-3.84 (m, 2H), 3.73 (s, 2H), 3.56-3.54 (m, 1H), 2.75-2.68 (m, 1H), 2.22-2.14 (m, 2H), 1.90-1.83 (m, 1H), 1.41 (s, 3H). |
| | It's obtained from the | | |
| | intermediate 50-1. | | |
| 51 | 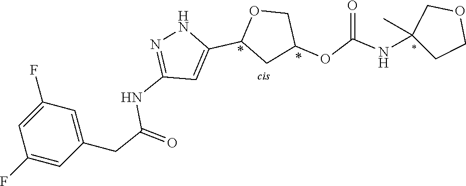 [SMILES: CC1(NC(=O)OC2COC(c3cc(NC(=O)Cc4cc(F)cc(F)c4)n[nH]3)C2)CCOC1] | 451.4 | ¹HNMR (400 MHz, METHANOL-d₄, ppm): δ 7.00-6.98 (m, 2H), 6.90-6.85 (m, 1H), 6.44 (s, 1H), 5.28-5.21 (m, 1H), 5.06-5.03 (m, 1H), 4.08-4.05 (m, 1H), 3.98-3.93 (m, 2H), 3.88-3.84 (m, 2H), 3.74 (s, 2H), 3.56-3.54 (m, 1H), 2.75-2.68 (m, 1H), 2.20-2.15 (m, 2H), 1.89-1.82 (m, 1H), 1.40 (s, 3H). |
| | It's obtained from the | | |
| | intermediate 50-2. | | |
| 52 | 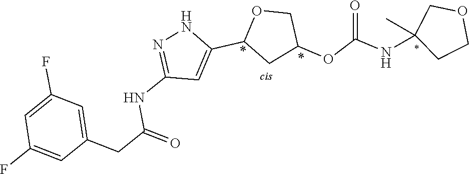 [SMILES: CC1(NC(=O)OC2COC(c3cc(NC(=O)Cc4cc(F)cc(F)c4)n[nH]3)C2)CCOC1] | 451.4 | ¹HNMR (400 MHz, METHANOL-d₄, ppm): δ 6.99-6.94 (m, 2H), 6.88-6.84 (m, 1H), 6.42 (s, 1H), 5.28-5.21 (m, 1H), 5.03-5.01 (m, 1H), 4.05-4.02 (m, 1H), 3.95-3.91 (m, 2H), 3.86-3.82 (m, 2H), 3.71 (s, 2H), 3.54-3.52 (m, 1H), 2.72-2.65 (m, 1H), 2.19-2.12 (m, 2H), 1.87-1.79 (m, 1H), 1.37 (s, 3H). |
| | It's obtained from the | | |
| | intermediate 50-3. | | |
| 53 | 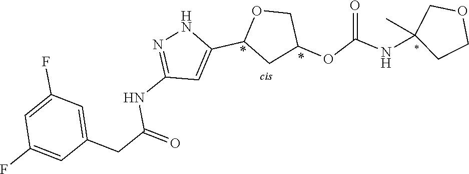 [SMILES: CC1(NC(=O)OC2COC(c3cc(NC(=O)Cc4cc(F)cc(F)c4)n[nH]3)C2)CCOC1] | 451.4 | ¹HNMR (400 MHz, METHANOL-d₄, ppm): δ 6.99-6.94 (m, 2H), 6.88-6.82 (m, 1H), 6.42 (s, 1H), 5.28-5.20 (m, 1H), 5.02-5.00 (m, 1H), 4.04-4.02 (m, 1H), 3.95-3.89 (m, 2H), 3.85-3.82 (m, 2H), 3.71 (s, 2H), 3.54-3.52 (m, 1H), 2.72-2.65 (m, 1H), 2.20-2.12 (m, 2H), 1.87-1.80 (m, 1H), 1.38 (s, 3H). |
| | It's obtained from the | | |
| | intermediate 50-4. | | |
| 54 | 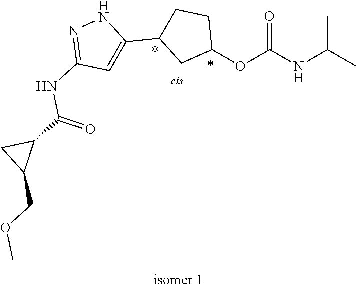 [SMILES: COC[C@H]1C[C@@H]1C(=O)Nc1cc(C2CCC(OC(=O)NC(C)C)C2)[nH]n1] | 367.4 | ¹HNMR (400 MHz, METHANOL-d₄, ppm): δ 6.36 (s, 1H), 5.28-5.22 (m, 1H), 5.01-4.98 (m, 1H), 4.05-4.02 (m, 1H), 3.96-3.92 (m, 1H), 3.69-3.66 (m, 1H), 3.50-3.46 (m, 1H), 3.34 (s, 3H), 3.26-3.23 (m, 1H), 2.77-2.66 (m, 1H), 2.14-2.09 (m, 1H), 1.76-1.63 (m, 2H), 1.21-1.17 (m, 1H), 1.12-0.93 (m, 6H), 0.91-0.87 (m, 1H). Chiral purity 85.96% ee, retention time 4.430 min. Chiral SFC analysis was performed on a Waters UPCC (CA-060), DAICEL CHIRALPAK ®OZ, 100*3.0 mm 3 μm column heated to 35° C., eluted with a mobile phase of CO₂ and 5-40% EtOH (0.1% DEA), flowing at 1.5 mL/min. |
| 55 | 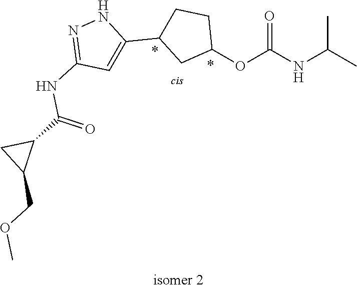 [SMILES: COC[C@H]1C[C@@H]1C(=O)Nc1cc(C2CCC(OC(=O)NC(C)C)C2)[nH]n1] | 367.4 | ¹HNMR (400 MHz, METHANOL-d₄, ppm): δ 6.35 (s, 1H), 5.28-5.21 (m, 1H), 5.01-4.99 (m, 1H), 4.05-4.03 (m, 1H), 3.96-3.92 (m, 1H), 3.71-3.64 (m, 1H), 3.51-3.47 (m, 1H), 3.34 (s, 3H), 3.26-3.23 (m, 1H), 2.74-2.67 (m, 1H), 2.14-2.11 (m, 1H), 1.74-1.67 (m, 2H), 1.21-1.19 (m, 1H), 1.11-0.94 (m, 6H), 0.91-0.87 (m, 1H). Chiral purity 94.72% ee, retention time 4.748 min. Chiral SFC analysis was performed on a Waters UPCC (CA-060), DAICEL CHIRALPAK ®OZ, 100*3.00 mm 3 μm column heated to 35° C., eluted with a mobile phase of CO₂ and 5-40% EtOH (0.1% DEA), flowing at 1.5 mL/ min. |
| 56 | 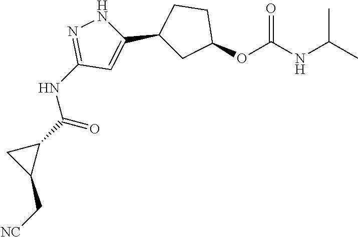 [SMILES: CC(C)NC(=O)O[C@@H]1CC[C@H](c2cc(NC(=O)[C@H]3C[C@@H]3CC#N)n[nH]2)C1] | 360.0 | ¹HNMR (400 MHz, DMSO-d₆, ppm): δ 12.02 (brs, 1H), 10.61 (s, 1H), 6.93-6.91 (m, 1H), 6.25 (s, 1H), 5.03-4.97 (m, 1H), 3.63-3.50 (m, 1H), 3.09-2.98 (m, 1H), 2.76-2.63 (m, 2H), 2.48-2.40 (m, 1H), 2.03-1.98 (m, 1H), 1.87-1.79 (m, 2H), 1.76-1.42 (m, 4H), 1.09-0.99 (m, 7H), 0.85-0.80 (m, 1H). |
| 57 | 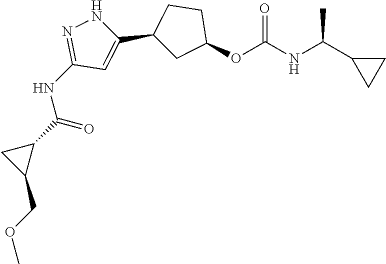 [SMILES: COC[C@H]1C[C@@H]1C(=O)Nc1cc([C@H]2CC[C@@H](OC(=O)N[C@@H](C)C3CC3)C2)[nH]n1] | 391.1 | ¹HNMR (400 MHz, METHANOL-d₄, ppm): δ 6.31 (s, 1H), 5.11-5.03 (m, 1H), 3.49-3.44 (m, 1H), 3.35 (s, 3H), 3.29-3.25 (m, 1H), 3.17-3.12 (m, 1H), 3.06-3.00 (m, 1H), 2.53-2.45 (m, 1H), 2.12-2.03 (m, 1H), 1.98-1.62 (m, 6H), 1.18-1.14 (m, 4H), 0.88-0.80 (m, 2H), 0.48-0.36 (m, 2H), 0.29-0.25 (m, 1H), 0.17-0.10 (m, 1H). |
| 58 | 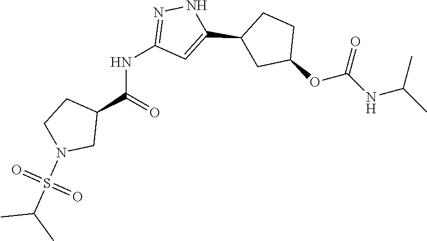 [SMILES: CC(C)NC(=O)O[C@@H]1CC[C@H](c2cc(NC(=O)[C@@H]3CCN(S(=O)(=O)C(C)C)C3)n[nH]2)C1] | 456.1 | ¹HNMR (400 MHz, METHANOL-d₄, ppm): δ 6.35 (s, 1H), 5.14-5.07 (m, 1H), 3.75-3.63 (m, 2H), 3.60-3.42 (m, 3H), 3.40-3.32 (m, 1H), 3.28-3.13 (m, 2H), 2.58-2.43 (m, 1H), 2.30-1.82 (m, 7H), 1.37-1.31 (m, 6H), 1.11 (d, J = 6.4 Hz, 6H). |
| 59 | 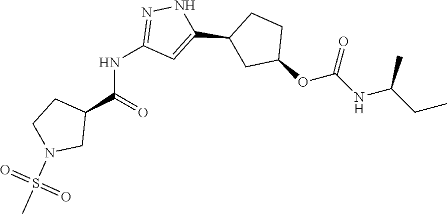 [SMILES: CC[C@H](C)NC(=O)O[C@@H]1CC[C@H](c2cc(NC(=O)[C@@H]3CCN(S(C)(=O)=O)C3)n[nH]2)C1] | 442.0 | ¹HNMR (400 MHz, DMSO-d₆, ppm): δ 12.08 (brs, 1H), 10.45 (brs, 1H), 6.90-6.88 (m, 1H), 6.34 (s, 1H), 5.07-4.92 (m, 1H), 3.51-3.46 (m, 1H), 3.43-3.20 (m, 5H), 3.15-3.03 (m, 1H), 2.91 (s, 3H), 2.51-2.42 (m, 1H), 2.20-2.10 (m, 1H), 2.09-1.97 (m, 2H), 1.96-1.85 (m, 1H), 1.75-1.50 (m, 3H), 1.39-1.30 (m, 2H), 1.00 (d, J = 6.4 Hz, 3H), 0.79 (t, J = 7.2 Hz, 3H). |
| 60 | 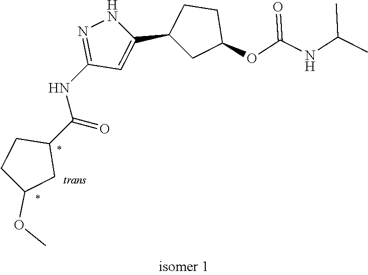 [SMILES: COC1CCC(C(=O)Nc2cc([C@H]3CC[C@@H](OC(=O)NC(C)C)C3)[nH]n2)C1] | 379.1 | ¹HNMR (400 MHz, METHANOL-d₄, ppm): δ 6.32 (s, 1H), 5.19-5.02 (m, 1H), 3.98-3.94 (m, 1H), 3.76-3.62 (m, 1H), 3.29 (s, 3H), 3.19-3.11 (m, 1H), 3.04-2.96 (m, 1H), 2.54-2.46 (m, 1H), 2.11-1.73 (m, 11H), 1.12-1.10 (m, 6H). Chiral purity 95.34% de, retention time: 4.570 min. Chiral SFC analysis was performed on a Waters UPCC, DAICEL CHIRALPAK ®OD-3, 250*4.6 mm 3 μm, column heated to 35° C., eluted with a mobile phase of CO₂ and 20.0% MeOH (0.1% DEA), flowing at 2.0 mL/min. |
| 61 | 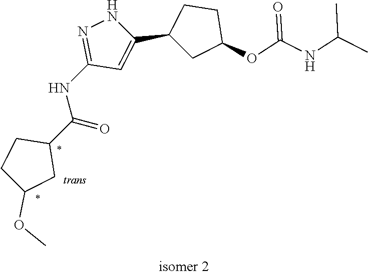 [SMILES: COC1CCC(C(=O)Nc2cc([C@H]3CC[C@@H](OC(=O)NC(C)C)C3)[nH]n2)C1] | 379.1 | ¹HNMR (400 MHz, METHANOL-d₄, ppm): δ 6.32 (s, 1H), 5.19-5.02 (m, 1H), 3.98-3.94 (m, 1H), 3.76-3.62 (m, 1H), 3.29 (s, 3H), 3.19-3.11 (m, 1H), 3.04-2.96 (m, 1H), 2.54-2.46 (m, 1H), 2.11-1.73 (m, 11H), 1.12-1.10 (m, 6H). Chiral purity 99.22% de, retention time: 5.836 min. Chiral SFC analysis was performed on a Waters UPCC, DAICEL CHIRALPAK ®OD-3, 250*4.6 mm 3 μm, column heated to 35° C., eluted with a mobile phase of CO₂ and 20.0% MeOH (0.1% DEA), flowing at 2.0 mL/min. |
| 62 | 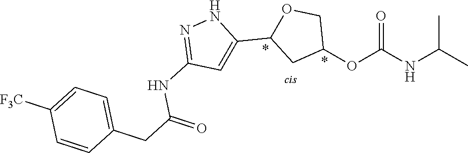 [SMILES: CC(C)NC(=O)OC1COC(c2cc(NC(=O)Cc3ccc(C(F)(F)F)cc3)n[nH]2)C1] | 441.4 | ¹HNMR (400 MHz, METHANOL-d₄, ppm): δ 7.65 (d, J = 8.0 Hz, 2H), 7.56 (d, J = 8.0 Hz, 2H), 6.43 (s, 1H), 5.28-5.23 (m, 1H), 5.03-5.00 (m, 1H), 4.06-4.03 (m, 1H), 3.97-3.93 (m, 1H), 3.81 (s, 2H), 3.71-3.65 (m, 1H), 2.74-2.67 (m, 1H), 2.15-2.10 (m, 1H), 1.16-0.90 (m, 6H). |
| | It's obtained from the | | |
| | intermediate 62-1. | | |
| 63 | 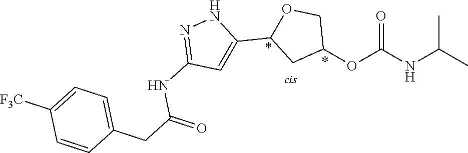 [SMILES: CC(C)NC(=O)OC1COC(c2cc(NC(=O)Cc3ccc(C(F)(F)F)cc3)n[nH]2)C1] | 441.4 | ¹HNMR (400 MHz, METHANOL-d₄, ppm): δ 7.65 (d, J = 8.4 Hz, 2H), 7.56 (d, J = 8.0 Hz, 2H), 6.43 (s, 1H), 5.28-5.23 (m, 1H), 5.04-5.00 (m, 1H), 4.06-4.04 (m, 1H), 3.97-3.93 (m, 1H), 3.81 (s, 2H), 3.71-3.65 (m, 1H), 2.74-2.67 (m, 1H), 2.15-2.11 (m, 1H), 1.16-0.93 (m, 6H). |
| | It's obtained from the | | |
| | intermediate 62-2. | | |
| 64 | 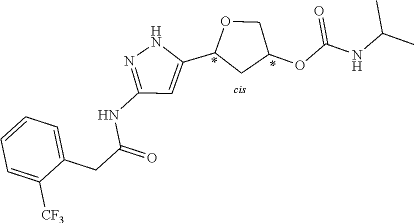 [SMILES: CC(C)NC(=O)OC1COC(c2cc(NC(=O)Cc3ccccc3C(F)(F)F)n[nH]2)C1] | 441.2 | ¹HNMR (400 MHz, METHANOL-d₄, ppm): δ 7.70 (d, J = 8.0 Hz, 1H), 7.62-7.58 (m, 1H), 7.50-7.44 (m, 2H), 6.41 (s, 1H), 5.28-5.21 (m, 1H), 5.00-4.97 (m, 1H), 4.03-4.01 (m, 1H), 3.95-3.91 (m, 3H), 3.67-3.64 (m, 1H), 2.72-2.64 (m, 1H), 2.13-2.08 (m, 1H), 1.14-0.94 (m, 6H). |
| | It's obtained from the | | |
| | intermediate 64-1. | | |
| 65 | 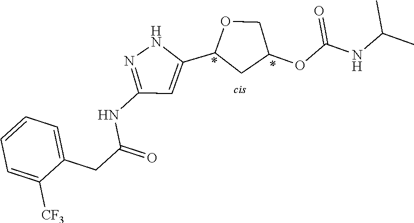 [SMILES: CC(C)NC(=O)OC1COC(c2cc(NC(=O)Cc3ccccc3C(F)(F)F)n[nH]2)C1] | 441.2 | ¹HNMR (400 MHz, METHANOL-d₄, ppm): δ 7.70 (d, J = 7.6 Hz, 1H), 7.62-7.58 (m, 1H), 7.50-7.44 (m, 2H), 6.40 (s, 1H), 5.28-5.21 (m, 1H), 5.01-4.97 (m, 1H), 4.04-4.01 (m, 1H), 3.96-3.91 (m, 3H), 3.67-3.62 (m, 1H), 2.72-2.65 (m, 1H), 2.13-2.08 (m, 1H), 1.14-0.95 (m, 6H). |
| | It's obtained from the | | |
| | intermediate 64-2. | | |
| 66 | 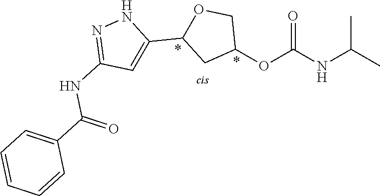 [SMILES: CC(C)NC(=O)OC1COC(c2cc(NC(=O)c3ccccc3)n[nH]2)C1] | 359.4 | ¹HNMR (400 MHz, METHANOL-d₄, ppm): δ 7.98-7.96 (m, 2H), 7.64-7.60 (m, 1H), 7.56-7.52 (m, 2H), 6.55 (s, 1H), 5.27-5.22 (m, 1H), 5.08-5.04 (m, 1H), 4.10-4.07 (m, 1H), 4.00-3.97 (m, 1H), 3.75-3.68 (m, 1H), 2.79-2.72 (m, 1H), 2.21-2.16 (m, 1H), 1.15-1.12 (m, 6H). |
| | It's obtained from the | | |
| | intermediate 66-1. | | |
| 67 | 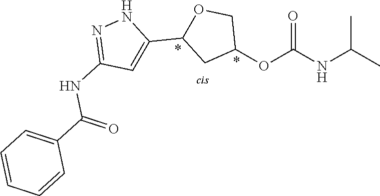 [SMILES: CC(C)NC(=O)OC1COC(c2cc(NC(=O)c3ccccc3)n[nH]2)C1] | 359.4 | ¹HNMR (400 MHz, METHANOL-d₄, ppm): δ 7.98-7.96 (m, 2H), 7.64-7.60 (m, 1H), 7.56-7.52 (m, 2H), 6.55 (s, 1H), 5.27-5.22 (m, 1H), 5.08-5.05 (m, 1H), 4.10-4.07 (m, 1H), 4.01-3.97 (m, 1H), 3.75-3.68 (m, 1H), 2.79-2.72 (m, 1H), 2.21-2.16 (m, 1H), 1.16-1.12 (m, 6H). |
| | It's obtained from the | | |
| | intermediate 66-2. | | |
| 68 | 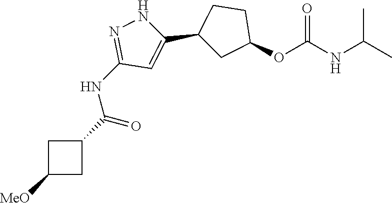 [SMILES: CO[C@H]1C[C@H](C(=O)Nc2cc([C@H]3CC[C@@H](OC(=O)NC(C)C)C3)[nH]n2)C1] | 365.4 | ¹H NMR (400 MHz, METHANOL-d₄, ppm): δ 6.35 (s, 1H), 5.09 (s, 1H), 4.21-4.04 (m, 1H), 3.69-3.67 (m, 1H), 3.24 (s, 3H), 3.19-3.09 (m, 2H), 2.53-2.48 (m, 3H), 2.25-2.20 (m, 2H), 2.18-2.10 (m, 1H), 1.97-1.73 (m, 4H), 1.12 (d, J = 6.4 Hz, 6H). |
| 70 | 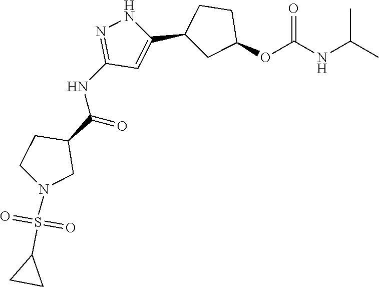 [SMILES: CC(C)NC(=O)O[C@@H]1CC[C@H](c2cc(NC(=O)[C@@H]3CCN(S(=O)(=O)C4CC4)C3)n[nH]2)C1] | 454.1 | ¹HNMR (400 MHz, METHANOL-d₄, ppm): δ 6.35 (s, 1H), 5.15-5.08 (m, 1H), 3.74-3.53 (m, 4H), 3.49-3.42 (m, 1H), 3.28-3.22 (m, 1H), 3.18-3.10 (m, 1H), 2.67-2.47 (m, 2H), 2.29-2.05 (m, 2H), 1.96-1.73 (m, 1H), 1.96-1.71 (m, 4H), 1.15-1.00 (m, 10H). |
| 71 | 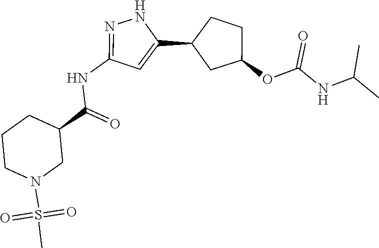 [SMILES: CC(C)NC(=O)O[C@@H]1CC[C@H](c2cc(NC(=O)[C@@H]3CCCN(S(C)(=O)=O)C3)n[nH]2)C1] | 442.0 | ¹HNMR (400 MHz, DMSO-d₆, ppm): δ 12.05 (brs, 1H), 10.40 (brs, 1H), 6.96-6.92 (m, 1H), 6.28 (s, 1H), 5.02-4.98 (m, 1H), 3.63-3.51 (m, 3H), 3.09-3.01 (m, 1H), 2.89 (s, 3H), 2.84-2.60 (m, 3H), 2.502-2.43 (m, 1H), 2.00-1.40 (m, 9H), 1.02 (d, J = 6.4 Hz, 6H). |
| 73 | 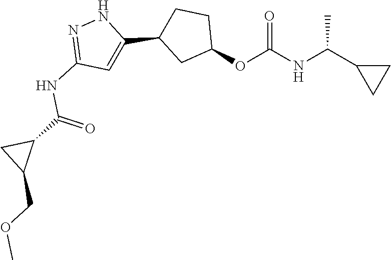 [SMILES: COC[C@H]1C[C@@H]1C(=O)Nc1cc([C@H]2CC[C@@H](OC(=O)N[C@H](C)C3CC3)C2)[nH]n1] | 391.1 | ¹HNMR (400 MHz, METHANOL-d₄, ppm): δ 6.26 (s, 1H), 5.14-5.06 (m, 1H), 3.53-3.46 (m, 1H), 3.35 (s, 3H), 3.28-3.13 (m, 2H), 3.04-2.96 (m, 1H), 2.55-2.45 (m, 1H), 2.18-2.10 (m, 1H), 2.00-1.62 (m, 6H), 1.25-1.20 (m, 1H), 1.17 (d, J = 6.8 Hz, 3H), 0.96-0.80 (m, 2H), 0.52-0.37 (m, 2H), 0.35-0.23 (m, 1H), 0.19-0.13 (m, 1H). |
| 74 | 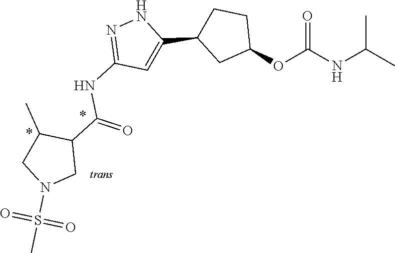 [SMILES: CC(C)NC(=O)O[C@@H]1CC[C@H](c2cc(NC(=O)C3CN(S(C)(=O)=O)CC3C)n[nH]2)C1] | 442.1 | ¹HNMR (400 MHz, DMSO-d₆, ppm): δ 12.09 (brs, 1H), 10.51 (brs, 1H), 6.93 (d, J = 7.6 Hz, 1H), 6.34 (s, 1H), 5.05-4.96 (m, 1H), 3.60-3.48 (m, 3H), 3.32-3.30 (m, 1H), 3.03-2.98 (m, 1H), 2.93 (s, 3H), 2.89-2.76 (m, 2H), 2.50-2.43 (m, 2H), 2.08-1.51 (m, 5H), 1.09-1.01 (m, 9H). |
| | It's obtained from the | | |
| | intermediate 74-1. | | |
| 75 | 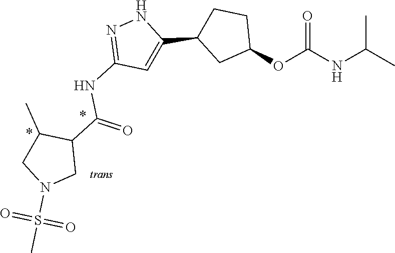 [SMILES: CC(C)NC(=O)O[C@@H]1CC[C@H](c2cc(NC(=O)C3CN(S(C)(=O)=O)CC3C)n[nH]2)C1] | 442.1 | ¹HNMR (400 MHz, DMSO-d₆, ppm): δ 12.09 (brs, 1H), 10.50 (brs, 1H), 6.93 (d, J = 7.2 Hz, 1H), 6.34 (s, 1H), 5.05-4.96 (m, 1H), 3.63-3.45 (m, 3H), 3.32-3.27 (m, 1H), 3.10-2.98 (m, 1H), 2.93 (s, 3H), 2.91-2.76 (m, 2H), 2.50-2.43 (m, 2H), 2.06-1.53 (m, 5H), 1.10-1.01 (m, 9H). |
| | It's obtained from the | | |
| | intermediate 74-2. | | |
| 76 | 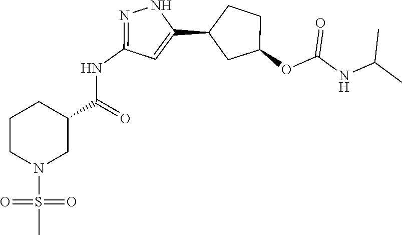 [SMILES: CC(C)NC(=O)O[C@@H]1CC[C@H](c2cc(NC(=O)[C@H]3CCCN(S(C)(=O)=O)C3)n[nH]2)C1] | 442.1 | ¹HNMR (400 MHz, DMSO-d₆, ppm): δ 12.05 (brs, 1H), 10.40 (brs, 1H), 6.94-6.85 (m, 1H), 6.30 (s, 1H), 5.03-4.95 (m, 1H), 3.68-3.48 (m, 3H), 3.08-2.98 (m, 1H), 2.89 (s, 3H), 2.78-2.71 (m, 1H), 2.68-2.58 (m, 2H), 2.50-2.40 (m, 1H), 2.08-1.41 (m, 9H), 0.98 (d, J = 6.4 Hz, 6H). |
| 77 | 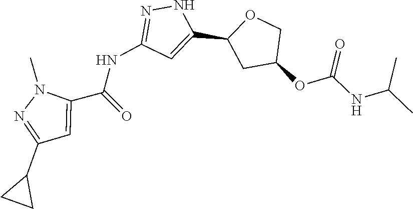 [SMILES: CC(C)NC(=O)O[C@@H]1CO[C@H](c2cc(NC(=O)c3cc(C4CC4)nn3C)n[nH]2)C1] | 403.4 | ¹HNMR (400 MHz, METHANOL-d₄, ppm): δ 6.62 (s, 1H), 6.49 (s, 1H), 5.30-5.23 (m, 1H), 5.04-5.01 (m, 1H), 4.07-4.04 (m, 4H), 3.97-3.93 (m, 1H), 3.72-3.65 (m, 1H), 2.76-2.69 (m, 1H), 2.16-2.12 (m, 1H), 1.95-1.88 (m, 1H), 1.12-1.00 (m, 6H), 0.97-0.92 (m, 2H), 0.74-0.70 (m, 2H). |
| 78 | 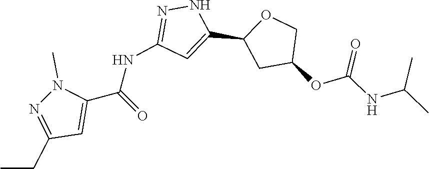 [SMILES: CCc1cc(C(=O)Nc2cc([C@@H]3C[C@H](OC(=O)NC(C)C)CO3)[nH]n2)n(C)n1] | 391.4 | ¹HNMR (400 MHz, METHANOL-d₄, ppm): δ 6.77 (s, 1H), 6.50 (s, 1H), 5.30-5.23 (m, 1H), 5.04-5.01 (m, 1H), 4.09-4.04 (m, 4H), 3.97-3.94 (m, 1H), 3.72-3.66 (m, 1H), 2.76-2.62 (m, 3H), 2.17-2.13 (m, 1H), 1.28-1.24 (m, 3H), 1.15-1.01 (m, 6H). |
| 79 | 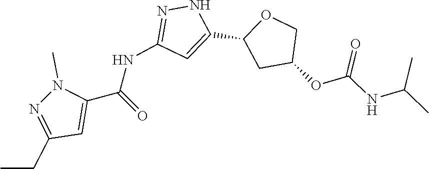 [SMILES: CCc1cc(C(=O)Nc2cc([C@H]3C[C@@H](OC(=O)NC(C)C)CO3)[nH]n2)n(C)n1] | 391.4 | ¹HNMR (400 MHz, METHANOL-d₄, ppm): δ 6.77 (s, 1H), 6.50 (s, 1H), 5.30-5.23 (m, 1H), 5.04-5.01 (m, 1H), 4.09-4.04 (m, 4H), 3.97-3.94 (m, 1H), 3.72-3.66 (m, 1H), 2.76-2.62 (m, 3H), 2.17-2.13 (m, 1H), 1.28-1.24 (m, 3H), 1.15-1.01 (m, 6H). |
| 80 | 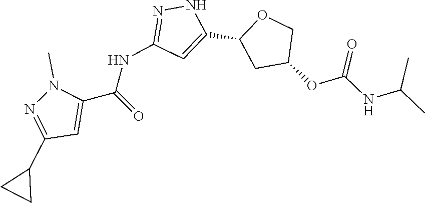 [SMILES: CC(C)NC(=O)O[C@H]1CO[C@@H](c2cc(NC(=O)c3cc(C4CC4)nn3C)n[nH]2)C1] | 403.4 | ¹H NMR (400 MHz, METHANOL-d₄, ppm): δ 6.62 (s, 1H), 6.49 (s, 1H), 5.23 (s, 1H), 5.06-4.99 (m, 1H), 4.07 (s, 3H), 4.06-4.02 (m, 1H), 3.99-3.92 (m, 1H), 3.74-3.63 (m, 1H), 2.77- 2.68 (m, 1H), 2.19-2.10 (m, 1H), 1.96-1.87 (m, 1H), 1.15-1.05 (m, 6H), 0.98-0.91 (m, 2H), 0.75-0.69 (m, 2H). |
| 81 | 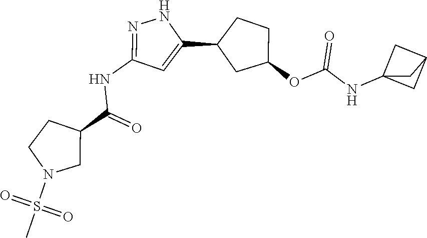 [SMILES: CS(=O)(=O)N1CC[C@@H](C(=O)Nc2cc([C@H]3CC[C@@H](OC(=O)NC45CC(C4)C5)C3)[nH]n2)C1] | 452.0 | ¹H NMR (400 MHz, DMSO-d₆, ppm): δ 12.10 (brs, 1H), 10.45 (brs, 1H), 7.75 (brs, 1H), 6.30 (s, 1H), 5.05-4.89 (m, 1H), 3.52-3.45 (m, 1H), 3.33-3.15 (m, 4H), 3.10-2.99 (m, 1H), 2.90 (s, 3H), 2.45-2.39 (m, 1H), 2.34 (s, 1H), 2.20-1.97 (m, 3H), 1.92-1.78 (m, 7H), 1.79-1.49 (m, 3H). |
| 82 | 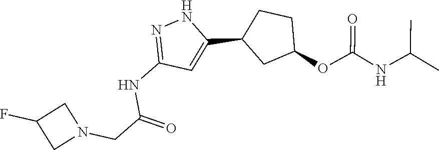 [SMILES: CC(C)NC(=O)O[C@@H]1CC[C@H](c2cc(NC(=O)CN3CC(F)C3)n[nH]2)C1] | 368.2 | ¹HNMR (400 MHz, DMSO-d₆, ppm): δ 9.90 (brs, 1H), 7.00-6.91 (brs, 1H), 6.28 (s, 1H), 5.29-5.09 (m, 1H), 5.03-4.95 (m, 1H), 3.72-3.64 (m, 2H), 3.62-3.54 (m, 1H), 3.35-3.26 (m, 2H), 3.25 (s, 2H), 3.10-2.82 (m, 2H), 2.48-2.40 (m, 1H), 2.05-1.83 (m, 2H), 1.78-1.65 (m, 3H), 1.03 (d, J = 6.4 Hz, 6H). |
| 83 | 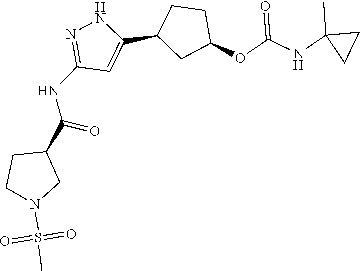 [SMILES: CC1(NC(=O)O[C@@H]2CC[C@H](c3cc(NC(=O)[C@@H]4CCN(S(C)(=O)=O)C4)n[nH]3)C2)CC1] | 440.1 | ¹HNMR (400 MHz, DMSO-d₆, ppm): δ 12.06 (brs, 1H), 10.44 (brs, 1H), 7.33 (s, 1H), 6.51 (s, 1H), 6.31 (s, 1H), 5.03-4.93 (m, 1H), 3.54-3.48 (m, 1H), 3.26-2.98 (m, 4H) 2.91 (s, 3H), 2.43-2.41 (m, 1H) 2.17-2.09 (m, 1H) 2.07-1.98 (m, 2H) 1.92-1.82 (m, 1H) 1.67-1.55 (m, 3H), 1.23 (s, 3H), 0.59-0.53 (m, 2H), 0.47-0.44 (m, 2H). |
| 85 | 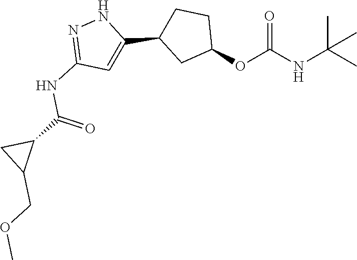 [SMILES: COCC1C[C@@H]1C(=O)Nc1cc([C@H]2CC[C@@H](OC(=O)NC(C)(C)C)C2)[nH]n1] | 379.1 | ¹HNMR (400 MHz, METHANOL-d₄, ppm): δ 6.31 (s, 1H), 5.10-5.02 (m, 1H), 3.49-3.45 (m, 1H), 3.35 (s, 3H), 3.29-3.25 (m, 1H), 3.16-3.12 (m, 1H), 2.55-2.45 (m, 1H), 2.15-2.03 (m, 1H), 1.99-1.85 (m, 3H), 1.79-1.72 (m, 2H), 1.67-1.62 (m, 1H), 1.28 (s, 9H), 1.19-1.14 (m, 1H), 0.89-0.84 (m, 1H). |
| 86 | 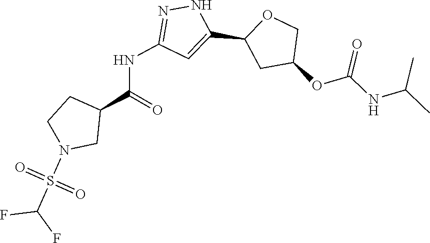 [SMILES: CC(C)NC(=O)O[C@@H]1CO[C@H](c2cc(NC(=O)[C@@H]3CCN(S(=O)(=O)C(F)F)C3)n[nH]2)C1] | 464.0 | ¹HNMR (400 MHz, DMSO-d₆, ppm): δ 12.08 (brs, 1H), 10.50 (brs, 1H), 7.13 (t, J = 52.4 Hz, 1H), 6.93 (d, J = 7.2 Hz, 1H), 6.31 (s, 1H), 5.04-4.93 (m, 1H) 3.69-3.64 (m, 1H), 3.61-3.50 (m, 3H), 3.48-3.42 (m, 1H), 3.28-3.22 (m, 1H), 3.12-2.98 (m, 1H), 2.47-2.42 (m, 1H), 2.24-1.83 (m, 4H), 1.79-1.52 (m, 3H), 1.03 (d, J = 6.4 Hz, 6H). |
| 87 | 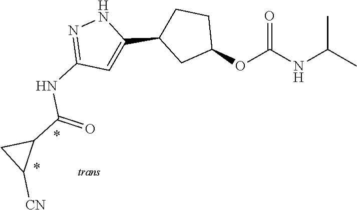 [SMILES: CC(C)NC(=O)O[C@@H]1CC[C@H](c2cc(NC(=O)C3CC3C#N)n[nH]2)C1] | 346.1 | ¹H NMR (400 MHz, DMSO-d₆, ppm): δ 12.13 (s, 1H), 10.89 (s, 1H), 6.93 (s, 1H), 6.26 (s, 1H), 4.98 (s, 1H), 3.57-3.55 (m, 1H), 3.05-3.02 (s, 1H), 2.46-2.43 (m, 2H), 2.03-1.32 (m, 8H), 1.03-1.01 (m, 6H). |
| 90 | 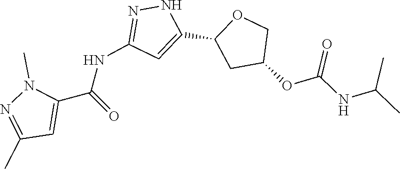 [SMILES: Cc1cc(C(=O)Nc2cc([C@H]3C[C@@H](OC(=O)NC(C)C)CO3)[nH]n2)n(C)n1] | 377.4 | ¹H NMR (400 MHz, METHANOL-d₄, ppm) δ 6.72 (s, 1H), 6.50 (s, 1H), 5.23 (s, 1H), 5.07-4.99 (m, 1H), 4.08 (s, 3H), 4.07-4.03 (m, 1H), 4.00-3.92 (m, 1H), 3.74-3.64 (m, 1H), 2.78- 2.67 (m, 1H), 2.26 (s, 3H), 2.19-2.10 (m, 1H), 1.15-1.05 (m, 6H). |
| 91 | 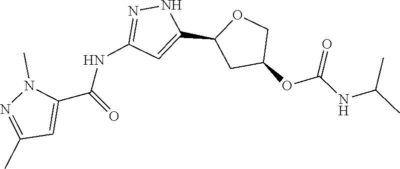 [SMILES: Cc1cc(C(=O)Nc2cc([C@@H]3C[C@H](OC(=O)NC(C)C)CO3)[nH]n2)n(C)n1] | 377.4 | ¹HNMR (400 MHz, METHANOL-d₄, ppm): δ 6.71 (s, 1H), 6.50 (s, 1H), 5.30-5.23 (m, 1H), 5.04-5.00 (m, 1H), 4.08-4.04 (m, 4H), 3.97-3.93 (m, 1H), 3.71-3.67 (m, 1H), 2.76-2.68 (m, 1H), 2.26 (s, 3H), 2.17-2.12 (m, 1H), 1.12-1.02 (m, 6H). |
| 92 | 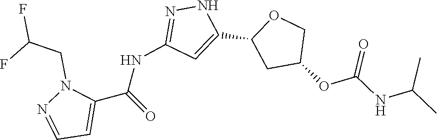 [SMILES: CC(C)NC(=O)O[C@H]1CO[C@@H](c2cc(NC(=O)c3ccnn3CC(F)F)n[nH]2)C1] | 413.4 | ¹HNMR (400 MHz, METHANOL-d₄, ppm): δ 7.62 (d, J = 2.0 Hz, 1H), 7.04 (d, J = 2.0 Hz, 1H), 6.52 (s, 1H), 6.39-6.09 (m, 1H), 5.30-5.23 (m, 1H), 5.04-4.96 (m, 3H), 4.07-3.94 (m, 2H), 3.72-3.66 (m, 1H), 2.76-2.69 (m, 1H), 2.18-2.13 (m, 1H), 1.15-1.02 (m, 6H). |
| 93 | 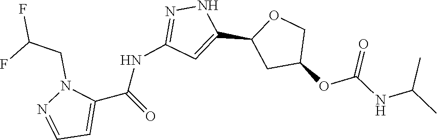 [SMILES: CC(C)NC(=O)O[C@@H]1CO[C@H](c2cc(NC(=O)c3ccnn3CC(F)F)n[nH]2)C1] | 413.2 | ¹HNMR (400 MHz, METHANOL-d₄, ppm): δ 7.62 (d, J = 2.0 Hz, 1H), 7.04 (d, J = 2.0 Hz, 1H), 6.52 (s, 1H), 6.39-6.09 (m, 1H), 5.30-5.23 (m, 1H), 5.04-4.96 (m, 3H), 4.07-3.94 (m, 2H), 3.70-3.67 (m, 1H), 2.76-2.69 (m, 1H), 2.17-2.14 (m, 1H), 1.15-1.02 (m, 6H). |
| 94 | 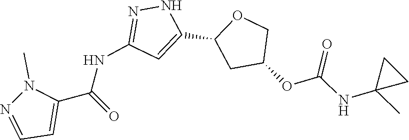 [SMILES: Cn1nccc1C(=O)Nc1cc([C@H]2C[C@@H](OC(=O)NC3(C)CC3)CO2)[nH]n1] | 375.4 | ¹HNMR (400 MHz, METHANOL-d₄, ppm) δ 7.51 (d, J = 2.0 Hz, 1H), 6.95 (d, J = 2.0 Hz, 1H), 6.49 (s, 1H), 5.23 (s, 1H), 5.07-5.00 (m, 1H), 4.16 (s, 3H), 4.07-4.01 (m, 1H), 3.98-3.92 (m, 1H), 2.79-2.67 (m, 1H), 2.19- 2.10 (m, 1H), 1.30 (s, 3H), 0.73-0.65 (m, 2H), 0.58-0.51 (m, 2H). |
| 95 | 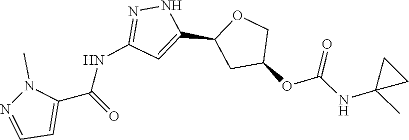 [SMILES: Cn1nccc1C(=O)Nc1cc([C@@H]2C[C@H](OC(=O)NC3(C)CC3)CO2)[nH]n1] | 375.4 | ¹HNMR (400 MHz, METHANOL-d₄, ppm): δ 7.51 (d, J = 2.0 Hz, 1H), 6.96 (s, 1H), 6.48 (s, 1H), 5.32-5.23 (m, 1H), 5.14-5.13 (m, 1H), 4.17 (s, 3H), 4.06-3.94 (m, 2H), 2.76-2.69 (m, 1H), 2.17-2.13 (m, 1H), 1.30 (s, 3H), 0.80-0.69 (m, 2H), 0.56-0.48 (m, 2H). |
| 96 | 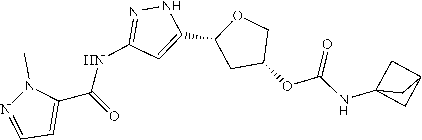 [SMILES: Cn1nccc1C(=O)Nc1cc([C@H]2C[C@@H](OC(=O)NC34CC(C3)C4)CO2)[nH]n1] | 387.4 | ¹HNMR (400 MHz, METHANOL-d₄, ppm): δ 7.52 (d, J = 2.0 Hz, 1H), 6.96 (d, J = 2.0 Hz, 1H), 6.50 (s, 1H), 5.23 (s, 1H), 5.08-4.99 (m, 1H), 4.17 (s, 3H), 4.08-4.02 (m, 1H), 4.00-3.92 (m, 1H), 2.78-2.67 (m, 1H), 2.36 (s, 1H), 2.20-2.07 (m, 1H), 2.04-1.84 (m, 6H). |
| 97 | 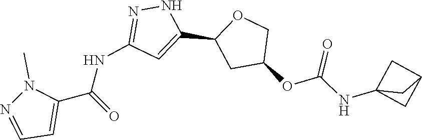 [SMILES: Cn1nccc1C(=O)Nc1cc([C@@H]2C[C@H](OC(=O)NC34CC(C3)C4)CO2)[nH]n1] | 387.4 | ¹HNMR (400 MHz, METHANOL-d₄, ppm): δ 7.52 (d, J = 2.0 Hz, 1H), 6.96 (d, J = 0.8 Hz, 1H), 6.49 (s, 1H), 5.32-5.23 (m, 1H), 5.11-5.04 (m, 1H), 4.17 (s, 3H), 4.07-3.97 (m, 2H), 2.77-2.70 (m, 1H), 2.36-2.32 (m, 1H), 2.15-2.08 (m, 1H), 1.99-1.86 (m, 6H). |
| 98 | 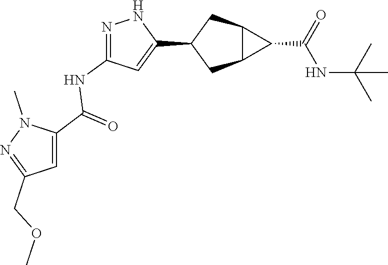 [SMILES: COCc1cc(C(=O)Nc2cc([C@H]3C[C@@H]4[C@H](C3)[C@H]4C(=O)NC(C)(C)C)[nH]n2)n(C)n1] | 415.2 | ¹HNMR (400 MHz, METHANOL-d₄, ppm): δ 6.95 (s, 1H), 6.34 (s, 1H), 4.44 (s, 2H), 4.14 (s, 3H), 3.53-3.48 (m, 1H), 3.38 (s, 3H), 2.53-2.46 (m, 2H), 2.14-2.07 (m, 2H), 1.81 (s, 2H), 1.34- 1.32 (m, 1H), 1.27 (s, 9H). |
| 99 | 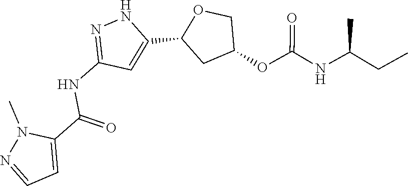 [SMILES: CC[C@H](C)NC(=O)O[C@H]1CO[C@@H](c2cc(NC(=O)c3ccnn3C)n[nH]2)C1] | 377.4 | ¹H NMR (400 MHz, METHANOL-d₄, ppm): δ 7.51 (d, J = 2.0 Hz, 1H), 6.95 (d, J = 2.0 Hz, 1H), 6.52 (s, 1H), 5.24 (s, 1H), 5.09-5.00 (m, 1H), 4.16 (s, 3H), 4.06 (d, J = 9.6 Hz, 1H), 3.98-3.94 (m, 1H), 3.51-3.46 (m, 1H), 2.76-2.70 (m, 1H), 2.18-2.13 (m, 1H), 1.45-1.43 (m, 2H), 1.08 (d, J = 6.4 Hz, 3H), 0.89 (t, J = 7.2 Hz, 3H). |
| 100 | 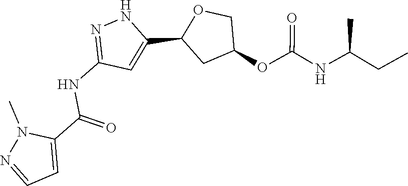 [SMILES: CC[C@H](C)NC(=O)O[C@@H]1CO[C@H](c2cc(NC(=O)c3ccnn3C)n[nH]2)C1] | 377.2 | ¹H NMR (400 MHz, METHANOL-d₄, ppm): δ 7.51 (d, J = 2.4 Hz, 1H), 6.95 (s, 1H), 6.51 (s, 1H), 5.23 (s, 1H), 5.07-5.03 (m, 1H), 4.16 (s, 3H), 4.06 (d, J = 10.4 Hz, 1H), 3.97-3.95 (m, 1H), 3.50-3.46 (m, 1H), 2.76-2.71 (m, 1H), 2.19-2.14 (m, 1H), 1.45-1.39 (m, 2H), 1.08 (d, J = 6.4 Hz, 3H), 0.88 (t, J = 7.2 Hz, 3H). |
| 101 | 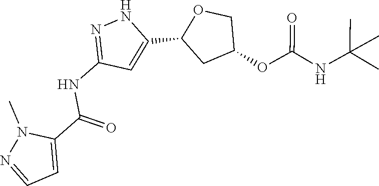 [SMILES: Cn1nccc1C(=O)Nc1cc([C@H]2C[C@@H](OC(=O)NC(C)(C)C)CO2)[nH]n1] | 377.4 | ¹H NMR (400 MHz, METHANOL-d₄, ppm): δ 7.51 (d, J = 2.0 Hz, 1H), 6.95 (d, J = 2.0 Hz, 1H), 6.53 (s, 1H), 5.20 (s, 1H), 5.05-4.98 (m, 1H), 4.16 (s, 3H), 4.04 (d, J = 10.4 Hz, 1H), 3.96-3.92 (m, 1H), 2.77-2.66 (m, 1H), 2.20-2.13 (m, 1H), 1.28 (s, 9H). |
| 102 | 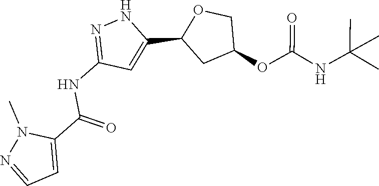 [SMILES: Cn1nccc1C(=O)Nc1cc([C@@H]2C[C@H](OC(=O)NC(C)(C)C)CO2)[nH]n1] | 377.2 | ¹H NMR (400 MHz, METHANOL-d₄, ppm): δ 7.51 (d, J = 2.4 Hz, 1H), 6.95 (d, J = 2.4 Hz, 1H), 6.52 (s, 1H), 5.20 (s, 1H), 5.05-5.01 (m, 1H), 4.16 (s, 3H), 4.04 (d, J = 10.4 Hz, 1H), 3.96-3.93 (m, 1H), 2.76-2.68 (m, 1H), 2.15-2.13 (m, 1H), 1.28 (s, 9H). |
| 103 | 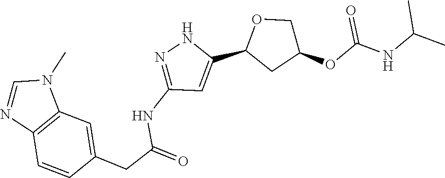 [SMILES: CC(C)NC(=O)O[C@@H]1CO[C@H](c2cc(NC(=O)Cc3ccc4ncn(C)c4c3)n[nH]2)C1] | 427.2 | ¹HNMR (400 MHz, DMSO-d₆, ppm): δ 12.32 (s, 1H), 10.57 (s, 1H), 8.13 (s, 1H), 7.61-7.54 (m, 1H), 7.48 (s, 1H), 7.24-7.14 (m, 1H), 7.12-7.01 (m, 1H), 6.44 (s, 1H), 5.14 (s, 1H), 4.85- 4.72 (m, 1H), 3.82 (s, 5H), 3.72 (s, 2H), 3.63-3.49 (m, 1H), 2.71-2.62 (m, 1H), 1.87 (s, 1H), 1.07-0.82 (m, 6H). |
| 104 | 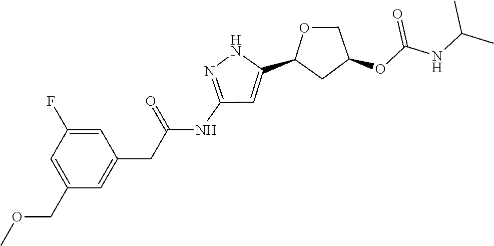 [SMILES: COCc1cc(F)cc(CC(=O)Nc2cc([C@@H]3C[C@H](OC(=O)NC(C)C)CO3)[nH]n2)c1] | 435.2 | ¹HNMR (400 MHz, DMSO-d₆, ppm): δ 12.35 (s, 1H), 10.61 (s, 1H), 7.18- 6.88 (m, 4H), 6.44 (s, 1H), 5.22-5.09 (m, 1H), 5.03-4.72 (m, 1H), 4.41 (s, 2H), 3.83 (s, 2H), 3.63 (s, 2H), 3.60- 3.51 (m, 1H), 3.31 (s, 3H), 2.72-2.60 (m, 1H), 1.88 (s, 1H), 1.10-0.87 (m, 6H). |
| 105 | 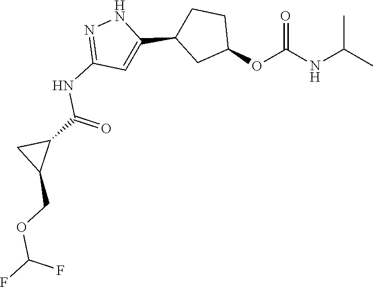 [SMILES: CC(C)NC(=O)O[C@@H]1CC[C@H](c2cc(NC(=O)[C@H]3C[C@@H]3COC(F)F)n[nH]2)C1] | 401.0 | ¹HNMR (300 MHz, METHANOL-d₄, ppm): δ 6.71 (brs, 1H), 6.38 (t, J = 75.3 Hz, 1H), 6.31 (brs, 1H), 5.15-5.03 (m, 1H), 3.93 (dd, J = 10.8, 6.0 Hz, 1H), 3.79-3.62 (m, 2H), 3.25-3.06 (m, 1H), 2.57-2.44 (m, 1H), 2.15-2.05 (m, 1H), 2.00-1.60 (m, 5H), 1.24-1.19 (m, 1H), 1.15-1.05 (m, 6H), 0.99-0.88 (m, 1H). |
| 106 | 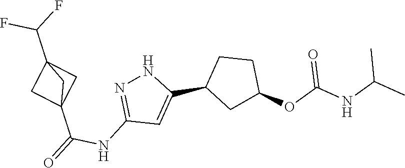 [SMILES: CC(C)NC(=O)O[C@@H]1CC[C@H](c2cc(NC(=O)C34CC(C(F)F)(C3)C4)n[nH]2)C1] | 397.0 | ¹HNMR (400 MHz, DMSO-d₆, ppm): δ 12.10 (brs, 1H), 10.22 (brs, 1H), 6.93 (d, J = 6.8 Hz, 1H), 6.28 (s, 1H), 6.07 (t, J = 56.4 Hz, 1H), 5.05-4.95 (m, 1H), 3.59-3.52 (m, 1H), 3.09-3.00 (m, 1H), 2.50-2.41 (m, 1H), 2.08 (s, 6H), 2.02-1.50 (m, 5H), 1.02 (d, J = 6.4 Hz, 6H). |
| 107 | 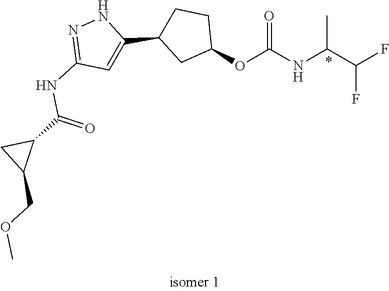 [SMILES: COC[C@H]1C[C@@H]1C(=O)Nc1cc([C@H]2CC[C@@H](OC(=O)NC(C)C(F)F)C2)[nH]n1] | 401.0 | ¹HNMR (400 MHz, METHANOL-d₄, ppm) δ 6.31 (s, 1H), 5.75 (t, J = 56.4 Hz, 1H), 5.18-5.08 (m, 1H), 3.99-3.85 (m, 1H), 3.49-3.45 (m, 1H), 3.35 (s, 3H), 3.29-3.22 (m, 1H), 3.19-3.10 (m, 1H), 2.59-2.48 (m, 1H), 2.13-2.06 (m, 1H), 1.99-1.72 (m, 5H), 1.68-1.60 (m, 1H), 1.19-1.10 (m, 4H), 0.88-0.84 (m, 1H). Chiral purity: 100% de, retention time: 14.461 min. Chiral SFC analysis was performed on a Waters UPCC, IG-3 column heated to 35° C., eluted with a mobile phase of CO₂ and gradient of 40% EtOH, flowing at 2.0 mL/min. |
| 108 | 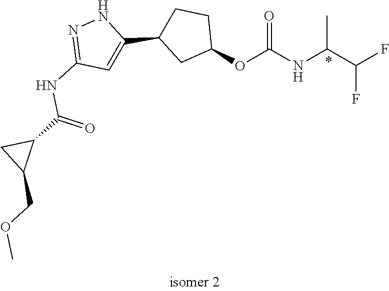 [SMILES: COC[C@H]1C[C@@H]1C(=O)Nc1cc([C@H]2CC[C@@H](OC(=O)NC(C)C(F)F)C2)[nH]n1] | 401.0 | ¹HNMR (400 MHz, METHANOL-d₄, ppm): δ 6.31 (s, 1H), 5.76 (t, J = 55.6 Hz, 1H), 5.16-5.09 (m, 1H), 3.99-3.85 (m, 1H), 3.49-3.45 (m, 1H), 3.35 (s, 3H), 3.29-3.25 (m, 1H), 3.22-3.10 (m, 1H), 2.54-2.47 (m, 1H), 2.15-2.06 (m, 1H), 1.97-1.72 (m, 5H), 1.69-1.60 (m, 1H), 1.19-1.14 (m, 4H), 0.89-0.82 (m, 1H). Chiral purity: 100% de, retention time: 24.754 min. Chiral SFC analysis was performed on a Waters UPCC, IG-3 column heated to 35° C., eluted with a mobile phase of CO₂ and gradient of 40% EtOH, flowing at 2.0 mL/min. |
| 109 | 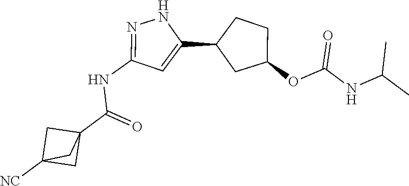 [SMILES: CC(C)NC(=O)O[C@@H]1CC[C@H](c2cc(NC(=O)C34CC(C#N)(C3)C4)n[nH]2)C1] | 372.1 | ¹H NMR (300 MHz, METHANOL-d₄, ppm): δ 6.30 (s, 1H), 5.13-5.05 (m, 1H), 3.79-3.61 (m, 1H), 3.21-3.06 (m, 1H), 2.56 (s, 6H), 2.53-2.43 (m, 1H), 2.16-2.02 (m, 1H), 2.00-1.68 (m, 4H), 1.20-1.12 (m, 3H). |
| 111 | 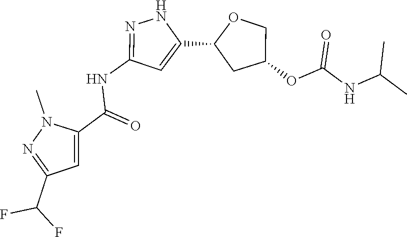 [SMILES: CC(C)NC(=O)O[C@H]1CO[C@@H](c2cc(NC(=O)c3cc(C(F)F)nn3C)n[nH]2)C1] | 413.4 | ¹H NMR (400 MHz, METHANOL-d₄, ppm): δ 7.17 (s, 1H), 6.77 (t, J = 54.8 Hz, 1H), 6.54 (s, 1H), 5.23 (s, 1H), 5.04-5.00 (m, 1H), 4.19 (s, 3H), 4.05 (d, J = 10.4 Hz, 1H), 3.97-3.93 (m, 1H), 3.70-3.67 (m, 1H), 2.76-2.69 (m, 1H), 2.17-2.13 (m, 1H), 1.13 (t, J = 3.6 Hz, 6H). |
| 112 | 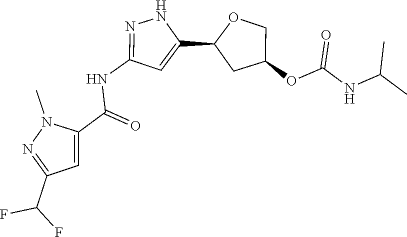 [SMILES: CC(C)NC(=O)O[C@@H]1CO[C@H](c2cc(NC(=O)c3cc(C(F)F)nn3C)n[nH]2)C1] | 413.4 | ¹HNMR (400 MHz, METHANOL-d₄, ppm): δ 7.17 (s, 1H), 6.90-6.63 (m, 1H), 6.53 (s, 1H), 5.30-5.23 (m, 1H), 5.04-5.01 (m, 1H), 4.19 (s, 3H), 4.06-3.96 (m, 1H), 3.95-3.93 (m, 1H), 3.71-3.67 (m, 1H), 2.76-2.69 (m, 1H), 2.17-2.12 (m, 1H), 1.15-1.02 (m, 6H). |
| 113 | 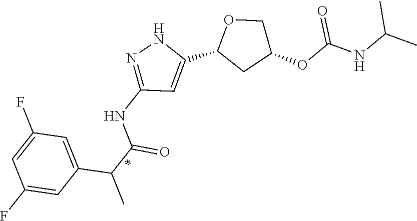 [SMILES: CC(C)NC(=O)O[C@H]1CO[C@@H](c2cc(NC(=O)C(C)c3cc(F)cc(F)c3)n[nH]2)C1] | 423.4 | ¹HNMR (400 MHz, METHANOL-d₄, ppm): δ 7.02-6.99 (m, 2H), 6.86- 6.80 (m, 1H), 6.39 (s, 1H), 5.20 (s, 1H), 5.00 (s, 1H), 4.03 (d, J = 10.0 Hz, 1H), 3.95-3.91 (m, 1H), 3.87-3.82 (m, 1H), 3.71-3.59 (m, 1H), 2.82-2.57 (m, 1H), 2.14-2.11 (m, 1H), 1.50 (d, J = 7.2 Hz, 3H), 1.09-1.06 (m, 6H). |
| | It's obtained from the | | |
| | intermediate 113-1. | | |
| 114 | 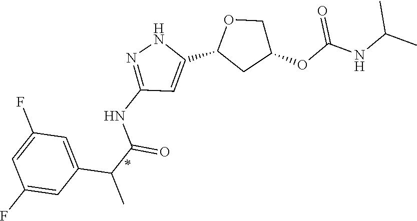 [SMILES: CC(C)NC(=O)O[C@H]1CO[C@@H](c2cc(NC(=O)C(C)c3cc(F)cc(F)c3)n[nH]2)C1] | 423.4 | ¹HNMR (400 MHz, METHANOL-d₄, ppm): δ 7.04-6.99 (m, 2H), 6.86-6.81 (m, 1H), 6.39 (s, 1H), 5.20 (s, 1H), 5.00 (s, 1H), 4.03 (d, J = 10.0 Hz, 1H), 3.98-3.90 (m, 1H), 3.87-3.83 (m, 1H), 3.69-3.59 (m, 1H), 2.72-2.56 (m, 1H), 2.14-2.11 (m, 1H), 1.49 (t, J = 7.2 Hz, 3H), 1.10-1.05 (m, 6H). |
| | It's obtained from the | | |
| | intermediate 113-2. | | |
| 115 | 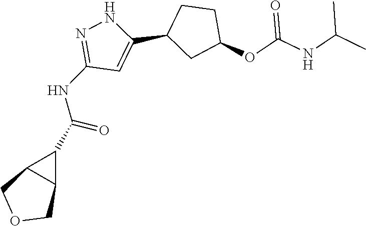 | 363.4 | ¹H NMR (400 MHz, METHANOL-d₄, ppm): δ 6.34 (s, 1H), 5.08 (s, 1H), 3.92 (d, J = 8.4 Hz, 2H), 3.76 (d, J = 8.4 Hz, 2H), 3.68-3.65 (m, 1H), 3.16- 3.12 (m, 1H), 2.58-2.39 (m, 1H), 2.16 (s, 2H), 2.09 (s, 1H), 1.96-1.67 (m, 5H), 1.13-1.05 (m, 6H). |
| 116 | 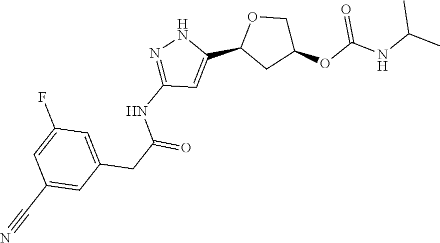 [SMILES: CC(C)NC(=O)O[C@@H]1CO[C@H](c2cc(NC(=O)Cc3cc(F)cc(C#N)c3)n[nH]2)C1] | 416.4 | ¹HNMR (400 MHz, DMSO-d₆, ppm): δ 12.37 (s, 1H), 10.66 (s, 1H), 7.76 (d, J = 8.0 Hz, 1H), 7.64 (s, 1H), 7.55 (d, J = 9.8 Hz, 1H), 7.06 (d, J = 7.8 Hz, 1H), 6.43 (s, 1H), 5.20-5.05 (m, 1H), 4.88-4.74 (m, 1H), 3.90-3.70 (m, 4H), 3.63-3.48 (m, 1H), 2.71-2.56 (m, 1H), 1.96-1.82 (m, 1H), 1.11- 0.94 (m, 6H). |
| 117 | 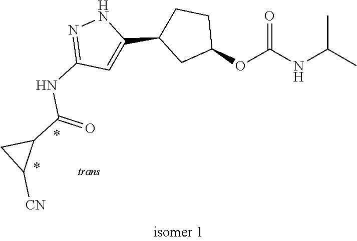 [SMILES: CC(C)NC(=O)O[C@@H]1CC[C@H](c2cc(NC(=O)C3CC3C#N)n[nH]2)C1] | 346.1 | ¹HNMR (400 MHz, DMSO-d₆, ppm): δ 12.12 (brs, 1H), 10.88 (s, 1H), 6.94-6.91 (m, 1H), 6.27 (s, 1H), 5.02-4.96 (m, 1H), 3.63-3.52 (m, 1H), 3.09-2.98 (m, 1H), 2.52-2.42 (m, 2H), 2.08-1.80 (m, 3H), 1.75-1.45 (m, 4H), 1.35-1.32 (m, 1H), 1.02 (d, J = 6.4 Hz, 6H). Chiral purity 99.80% de, retention time 4.694 min. Chiral SFC analysis was performed on a OD-3 column heated to 35° C., eluted with a mobile phase of CO₂ and 20% MeOH, flowing at 2.0 mL/min. |
| 118 | 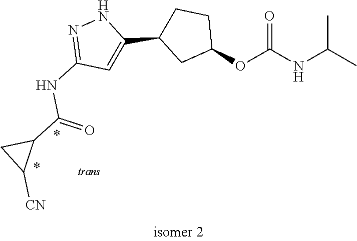 [SMILES: CC(C)NC(=O)O[C@@H]1CC[C@H](c2cc(NC(=O)C3CC3C#N)n[nH]2)C1] | 346.0 | ¹HNMR (400 MHz, DMSO-d₆, ppm) δ 12.13 (brs, 1H), 10.89 (s, 1H), 6.94-6.91 (s, 1H), 6.27 (s, 1H), 5.02-4.96 (m, 1H), 3.62-3.52 (m, 1H), 3.09-3.01 (m, 1H), 2.52-2.41 (m, 2H), 2.09-1.82 (m, 3H), 1.78-1.47 (m, 4H), 1.35-1.31 (m, 1H), 1.02 (d, J = 6.4 Hz, 6H). Chiral purity 98.70% de, retention time 5.989 min. Chiral SFC analysis was performed on a OD-3 column heated to 35° C., eluted with a mobile phase of CO₂ and 20% MeOH, flowing at 2.0 mL/min. |
| 119 | 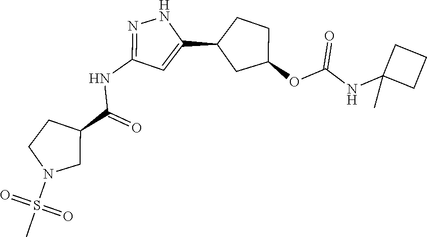 [SMILES: CC1(NC(=O)O[C@@H]2CC[C@H](c3cc(NC(=O)[C@@H]4CCN(S(C)(=O)=O)C4)n[nH]3)C2)CCC1] | 454.0 | ¹HNMR (400 MHz, [symbol] -d₄, ppm): δ 6.36 (brs, 1H), 5.07 (brs, 1H), 3.62-3.52 (m, 2H), 3.50-3.37 (m, 2H), 3.28-3.21 (m, 1H), 3.19-3.11 (m, 1H), 2.92 (s, 3H), 2.55-2.47 (m, 1H), 2.32-2.16 (m, 4H), 2.13-2.07 (m, 1H), 1.99-1.75 (m, 8H), 1.39 (s, 3H). |
| 121 | 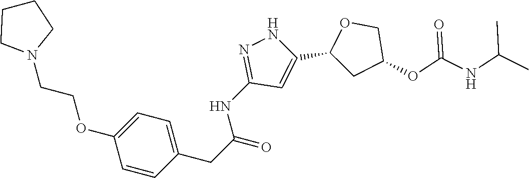 [SMILES: CC(C)NC(=O)O[C@H]1CO[C@@H](c2cc(NC(=O)Cc3ccc(OCCN4CCCC4)cc3)n[nH]2)C1] | 486.6 | ¹HNMR (400 MHz, METHANOL-d₄, ppm): δ 7.31 (d, J = 8.4 Hz, 2H), 7.00-6.98 (m, 2H), 6.41 (s, 1H), 5.28-5.21 (m, 1H), 4.98-4.85 (m, 1H), 4.32-4.30 (m, 2H), 4.03-3.90 (m, 2H), 3.71-3.63 (m, 7H), 3.25-3.18 (m, 2H), 2.72-2.65 (m, 1H), 2.20-2.03 (m, 5H), 1.14-0.96 (m, 6H). |
| 122 | 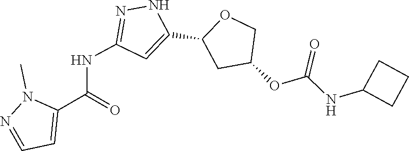 [SMILES: Cn1nccc1C(=O)Nc1cc([C@H]2C[C@@H](OC(=O)NC3CCC3)CO2)[nH]n1] | 375.4 | ¹HNMR (400 MHz, METHANOL-d₄, ppm): δ 7.52 (d, J = 2.4 Hz, 1H), 6.97 (d, J = 2.4 Hz, 1H), 6.50 (s, 1H), 5.22 (s, 1H), 5.09-5.00 (m, 1H), 4.17 (s, 3H), 4.14-4.02 (m, 2H), 3.99-3.94 (m, 1H), 2.77-2.63 (m, 1H), 2.28-2.10 (m, 3H), 1.93-1.88 (m, 2H), 1.67-1.50 (m, 2H) |
| 123 | 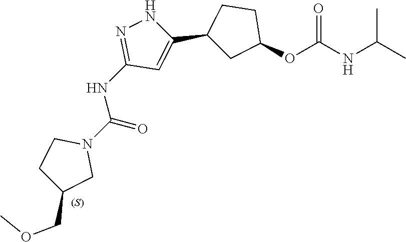 | 394.1 | ¹HNMR (400 MHz, METHANOL-d₄, ppm): δ 6.15 (s, 1H), 5.15-5.07 (m, 1H), 3.75-3.66 (m, 1H), 3.63-3.52 (m, 2H), 3.47-3.35 (m, 6H), 3.25-3.20 (m, 1H), 3.16-3.05 (m, 1H), 2.64-2.43 (m, 2H), 2.15-2.04 (m, 2H), 2.00-1.72 (m, 5H), 1.12 (d, J = 6.4 Hz, 6H). |
| 124 | 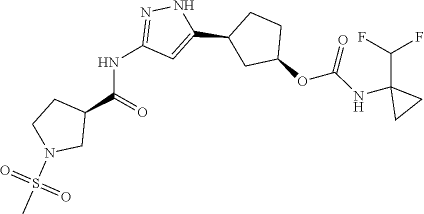 [SMILES: CS(=O)(=O)N1CC[C@@H](C(=O)Nc2cc([C@H]3CC[C@@H](OC(=O)NC4(C(F)F)CC4)C3)[nH]n2)C1] | 476.0 | ¹HNMR (400 MHz, METHANOL-d₄, ppm): δ 6.37 (s, 1H), 5.82 (t, J = 57.2 Hz, 1H), 5.21-5.10 (m, 1H), 3.65-3.38 (m, 4H), 3.26-3.10 (m, 2H), 2.92 (s, 3H), 2.56-2.46 (m, 1H), 2.31-2.08 (m, 3H), 2.02-1.72 (m, 4H), 1.08-1.03 (m, 2H), 0.90-0.80 (m, 2H). |
| 125 | 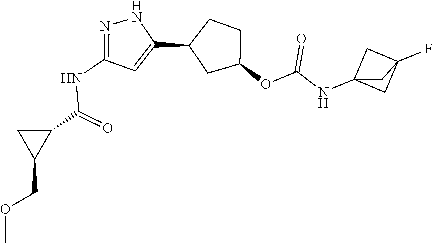 [SMILES: COC[C@H]1C[C@@H]1C(=O)Nc1cc([C@H]2CC[C@@H](OC(=O)NC34CC(F)(C3)C4)C2)[nH]n1] | 407.0 | ¹HNMR (400 MHz, METHANOL-d₄, ppm): δ 6.31 (s, 1H), 5.15-5.07 (m, 1H), 3.52-3.44 (m, 1H), 3.35 (s, 3H), 3.27-3.24 (m, 1H), 3.21-3.10 (m, 1H), 2.57-2.43 (m, 1H), 2.27 (s, 6H), 2.12-1.60 (m, 7H), 1.20-1.12 (m, 1H), 0.92-0.83 (m, 1H). |
| 126 | 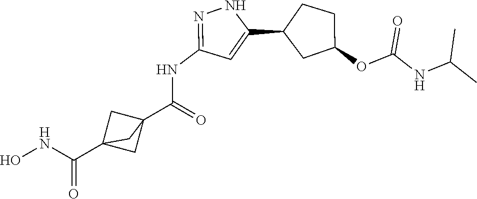 [SMILES: CC(C)NC(=O)O[C@@H]1CC[C@H](c2cc(NC(=O)C34CC(C(=O)NO)(C3)C4)n[nH]2)C1] | 406.1 | ¹HNMR (400 MHz, METHANOL-d₄, ppm): δ 6.34 (s, 1H), 5.13-5.05 (m, 1H), 3.79-3.62 (m, 1H), 3.20-3.11 (m, 1H), 2.60-2.48 (m, 1H), 2.31 (s, 6H), 2.15-2.05 (m, 1H), 2.03-1.74 (m, 4H), 1.16-1.06 (m, 6H). |
| 127 | 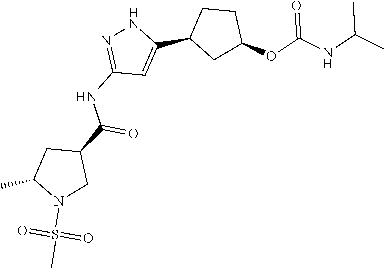 [SMILES: CC(C)NC(=O)O[C@@H]1CC[C@H](c2cc(NC(=O)[C@@H]3C[C@@H](C)N(S(C)(=O)=O)C3)n[nH]2)C1] | 442.0 | ¹H NMR (300 MHz, DMSO-d₆, ppm): δ 12.08 (brs, 1H), 10.46 (brs, 1H), 6.99-6.85 (m, 1H), 6.30 (s, 1H), 5.02-4.90 (m, 1H), 3.90-3.85 (m, 1H), 3.65-3.43 (m, 2H), 3.38-3.35 (m, 1H), 3.10-2.98 (m, 1H), 2.91 (s, 3H), 2.50-2.43 (m, 1H), 2.20-1.40 (m, 8H), 1.18 (d, J = 6.3 Hz, 3H), 1.02 (d, J = 6.6 Hz, 6H). |
| 128 | 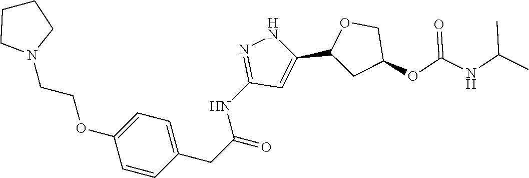 | 486.5 | ¹HNMR (400 MHz, METHANOL-d₄, ppm): δ 7.31 (d, J = 8.8 Hz, 2H), 6.98 (d, J = 8.8 Hz, 2H), 6.41 (s, 1H), 5.28-5.20 (m, 1H), 4.98-4.94 (m, 1H), 4.32-4.29 (m, 2H), 4.03-3.90 (m, 2H), 3.71-3.63 (m, 7H), 3.22-3.18 (m, 2H), 2.72-2.65 (m, 1H), 2.18-2.03 (m, 5H), 1.10-0.93 (m, 6H). |
| 129 | 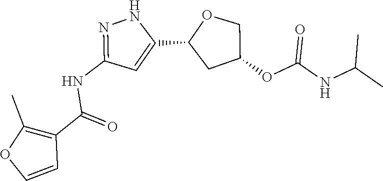 [SMILES: Cc1occc1C(=O)Nc1cc([C@H]2C[C@@H](OC(=O)NC(C)C)CO2)[nH]n1] | 363.4 | ¹HNMR (400 MHz, METHANOL-d₄, ppm): δ 7.43 (d, J = 2.0 Hz, 1H), 6.86 (d, J = 2.0 Hz, 1H), 6.44 (s, 1H), 5.31-5.23 (m, 1H), 5.06-5.02 (m, 1H), 4.07-3.95 (m, 2H), 3.70-3.67 (m, 1H), 2.76-2.69 (m, 1H), 2.59 (s, 3H), 2.18-2.14 (m, 1H), 1.15-0.95 (m, 6H). |
| 130 | 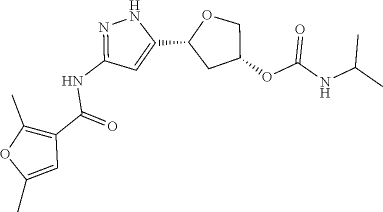 [SMILES: Cc1cc(C(=O)Nc2cc([C@H]3C[C@@H](OC(=O)NC(C)C)CO3)[nH]n2)c(C)o1] | 378.4 | ¹HNMR (400 MHz, METHANOL-d₄, ppm): δ 6.42 (s, 2H), 5.23 (s, 1H), 5.05-5.03 (m, 1H), 4.06 (d, J = 10.0 Hz, 1H), 3.97-3.94 (m, 1H), 3.70-3.66 (m, 1H), 2.74-2.71 (m, 1H), 2.54 (s, 3H), 2.27 (s, 3H), 2.16-2.13 (m, 1H), 1.12-1.09 (m, 6H). |
| 131 | 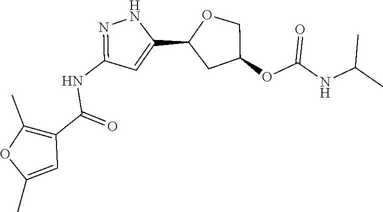 [SMILES: Cc1cc(C(=O)Nc2cc([C@@H]3C[C@H](OC(=O)NC(C)C)CO3)[nH]n2)c(C)o1] | 378.4 | ¹HNMR (400 MHz, METHANOL-d₄, ppm): δ 6.42 (s, 2H), 5.23 (s, 1H), 5.05-5.03 (m, 1H), 4.06 (d, J = 10.0 Hz, 1H), 3.97-3.94 (m, 1H), 3.70-3.66 (m, 1H), 2.74-2.71 (m, 1H), 2.54 (s, 3H), 2.27 (s, 3H), 2.16-2.13 (m, 1H), 1.12-1.09 (m, 6H). |
| 132 | 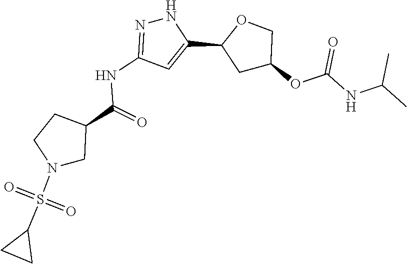 [SMILES: CC(C)NC(=O)O[C@@H]1CO[C@H](c2cc(NC(=O)[C@@H]3CCN(S(=O)(=O)C4CC4)C3)n[nH]2)C1] | 456.4 | ¹H NMR (400 MHz, DMSO-d₆, ppm): δ 12.36 (s, 1H), 10.55 (s, 1H), 7.11 (d, J = 7.6 Hz, 1H), 6.48 (s, 1H), 5.21- 5.10 (m, 1H), 4.85-4.75 (m, 1H), 3.92-3.77 (m, 2H), 3.63-3.51 (m, 2H), 3.45-3.35 (m, 2H), 3.30-3.17 (m, 2H), 2.74-2.62 (m, 2H), 2.20- 2.10 (m, 1H), 2.09-2.00 (m, 1H), 1.94-1.84 (m, 1H), 1.04 (d, J = 6.4 Hz, 6H), 0.99-0.92 (m, 4H). |
| 133 | 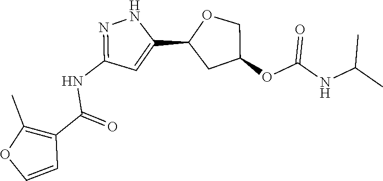 [SMILES: Cc1occc1C(=O)Nc1cc([C@@H]2C[C@H](OC(=O)NC(C)C)CO2)[nH]n1] | 363.4 | ¹H NMR (400 MHz, METHANOL-d₄, ppm): δ 7.41 (s, 1H), 6.85 (s, 1H), 6.58 (s, 1H), 5.23 (s, 1H), 5.03 (s, 1H), 4.05-4.03 (m, 1H), 3.95-3.90 (m, 1H), 3.71-3.68 (m, 1H), 2.78-2.61 (m, 1H), 2.58 (s, 3H), 2.16-2.12 (m, 1H), 1.12 (d, J = 3.6 Hz, 6H) |
| 134 | 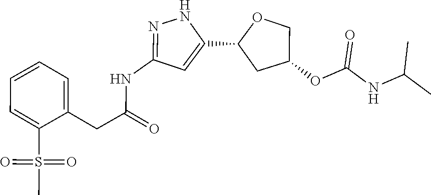 [SMILES: CC(C)NC(=O)O[C@H]1CO[C@@H](c2cc(NC(=O)Cc3ccccc3S(C)(=O)=O)n[nH]2)C1] | 451.4 | ¹H NMR (400 MHz, METHANOL-d₄, ppm): δ 8.06-8.04 (m, 1H), 7.69-7.65 (m, 1H), 7.60-7.44 (m, 2H), 6.47 (s, 1H), 5.20 (s, 1H), 4.97 (s, 1H), 4.28 (s, 2H), 3.99-3.91 (m, 2H), 3.70-3.57 (m, 1H), 3.20 (s, 3H), 2.74-2.58 (m, 1H), 2.10-2.08 (m, 1H), 1.08-1.05 (m, 6H). |
| 135 | 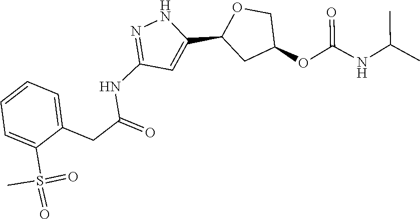 [SMILES: CC(C)NC(=O)O[C@@H]1CO[C@H](c2cc(NC(=O)Cc3ccccc3S(C)(=O)=O)n[nH]2)C1] | 451.2 | ¹HNMR (400 MHz, METHANOL-d₄, ppm): δ 8.06-8.04 (m, 1H), 7.70-7.66 (m, 1H), 7.58-7.51 (m, 2H), 6.38 (s, 1H), 5.28-5.20 (m, 1H), 5.01-4.98 (m, 1H), 4.28 (s, 2H), 4.04-3.91 (m, 2H), 3.69-3.62 (m, 1H), 3.20 (s, 3H), 2.73-2.65 (m, 1H), 2.13-2.08 (m, 1H), 1.12-0.96 (m, 6H). |
| 138 | 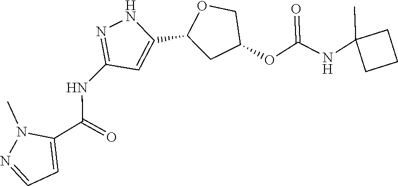 [SMILES: Cn1nccc1C(=O)Nc1cc([C@H]2C[C@@H](OC(=O)NC3(C)CCC3)CO2)[nH]n1] | 389.4 | ¹HNMR (400 MHz, METHANOL-d₄, ppm): δ 7.51 (d, J = 2.4 Hz, 1H), 6.95 (d, J = 2.4 Hz, 1H), 6.52 (s, 1H), 5.23-5.21 (m, 1H), 5.07-5.03 (m, 1H), 4.16 (s, 3H), 4.06 (d, J = 10.4 Hz, 1H), 3.97-3.95 (m, 1H), 2.76-2.71 (m, 1H), 2.28-2.14 (m, 3H), 1.91-1.76 (m, 4H), 1.38 (s, 3H). |
| 139 | 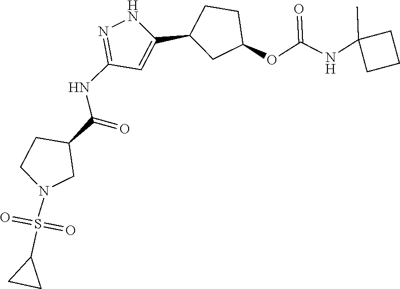 [SMILES: CC1(NC(=O)O[C@@H]2CC[C@H](c3cc(NC(=O)[C@@H]4CCN(S(=O)(=O)C5CC5)C4)n[nH]3)C2)CCC1] | 480.2 | ¹HNMR (400 MHz, METHANOL-d₄, ppm): δ 6.36 (s, 1H), 5.12-5.05 (m, 1H), 3.69-3.53 (m, 3H), 3.51-3.42 (m, 1H), 3.28-3.22 (m, 1H), 3.20-3.11 (m, 1H), 2.64-2.47 (m, 2H), 2.35-2.03 (m, 5H), 1.97-1.70 (m, 8H), 1.39 (s, 3H), 1.11-1.00 (m, 4H). |
| 140 | 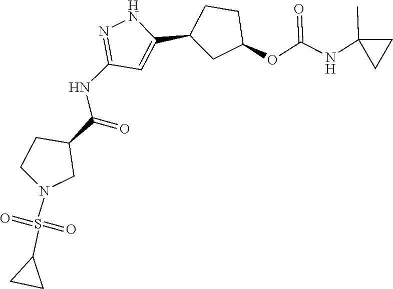 [SMILES: CC1(NC(=O)O[C@@H]2CC[C@H](c3cc(NC(=O)[C@@H]4CCN(S(=O)(=O)C5CC5)C4)n[nH]3)C2)CC1] | 466.0 | ¹HNMR (400 MHz, METHANOL-d₄, ppm): δ 6.33 (s, 1H), 5.16-5.08 (m, 1H), 3.72-3.41 (m, 4H), 3.28-3.11 (m, 2H), 2.67-2.46 (m, 2H), 2.31-2.09 (m, 3H), 1.99-1.70 (m, 4H), 1.31 (s, 3H), 1.13-1.00 (m, 4H), 0.71-0.64 (m, 2H), 0.59-0.54 (m, 2H). |
| 141 | 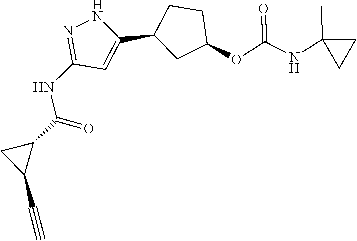 [SMILES: C#C[C@H]1C[C@@H]1C(=O)Nc1cc([C@H]2CC[C@@H](OC(=O)NC3(C)CC3)C2)[nH]n1] | 357.2 | ¹H NMR (300 MHz, DMSO-d₆, ppm): δ 12.06 (brs, 1H), 10.71 (brs, 1H), 7.33 (brs, 1H), 6.25 (s, 1H), 5.10-4.92 (m, 1H), 3.15-2.92 (m, 1H), 2.81 (d, J = 2.1 Hz, 1H), 2.47-2.37 (m, 1H), 2.20-2.10 (m, 1H), 2.05-1.92 (m, 1H), 1.91-1.76 (m, 1H), 1.75-1.45 (m, 4H), 1.22 (s, 3H), 1.20-1.12 (m, 1H), 1.12-1.02 (m, 1H), 0.62-0.54 (m, 2H), 0.48-0.42 (m, 2H). |
| 142 | 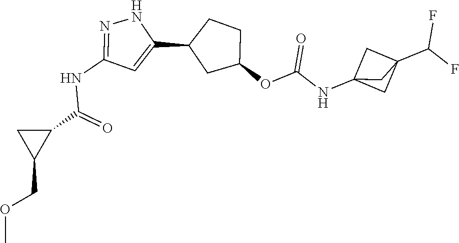 [SMILES: COC[C@H]1C[C@@H]1C(=O)Nc1cc([C@H]2CC[C@@H](OC(=O)NC34CC(C(F)F)(C3)C4)C2)[nH]n1] | 439.0 | ¹HNMR (400 MHz, DMSO-d₆, ppm): δ 12.00 (brs, 1H), 10.49 (brs, 1H), 7.92 (brs, 1H), 6.28-5.99 (m, 2H), 5.04-4.94 (m, 1H), 3.37-3.32 (m, 1H), 3.23 (s, 3H), 3.18-3.13 (m, 1H), 3.05-3.03 (m, 1H), 2.50-2.41 (m, 1H), 1.97-1.83 (m, 8H), 1.79-1.52 (m, 4H), 1.49-1.40 (m, 1H), 0.97-0.92 (m, 1H), 0.73-0.71 (m, 1H). |
| 143 | 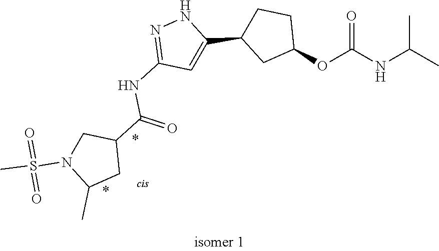 [SMILES: CC(C)NC(=O)O[C@@H]1CC[C@H](c2cc(NC(=O)C3CC(C)N(S(C)(=O)=O)C3)n[nH]2)C1] | | ¹HNMR (400 MHz, METHANOL-d₄, ppm): δ 6.36 (s, 1H), 5.16-5.07 (m, 1H), 3.91-3.60 (m, 3H), 3.56-3.47 (m, 1H), 3.20-3.07 (m, 2H), 2.92 (s, 3H), 2.58-2.43 (m, 2H), 2.17-1.70 (m, 6H), 1.37 (d, J = 6.0 Hz, 3H), 1.12 (d, J = 6.4 Hz, 6H). Chiral purity 99.42% de, retention time: 3.415 min. Chiral SFC analysis was performed on a Waters UPCC, column: DAICEL CHIRALPAK ®OD-3, 250*4.6 mm 3 μm, column heated to 35° C., eluted with a mobile phase of CO₂ and 5-20% MeOH (0.1% DEA), flowing at 2.0 mL/min. |
| 144 | 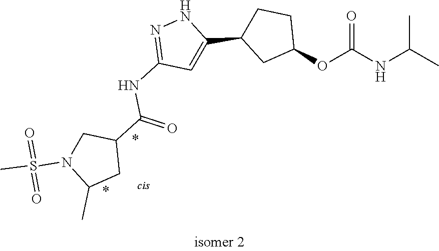 [SMILES: CC(C)NC(=O)O[C@@H]1CC[C@H](c2cc(NC(=O)C3CC(C)N(S(C)(=O)=O)C3)n[nH]2)C1] | | ¹HNMR (400 MHz, METHANOL-d₄, ppm): δ 6.36 (s, 1H), 5.16-5.09 (m, 1H), 3.91-3.63 (m, 3H), 3.55-3.48 (m, 1H), 3.18-3.07 (m, 2H), 2.92 (s, 3H), 2.57-2.44 (m, 2H), 2.11-1.73 (m, 6H), 1.37 (d, J = 6.0 Hz, 3H), 1.12 (d, J = 6.4 Hz, 6H). Chiral purity 99.64% de, retention time: 3.870 min. Chiral SFC analysis was performed on a Waters UPCC, column: DAICEL CHIRALPAK ®OD-3, 250*4.6 mm 3 μm, column heated to 35° C., eluted with a mobile phase of CO₂ and 5-20% MeOH (0.1% DEA), flowing at 2.0 mL/min. |
| 145 | 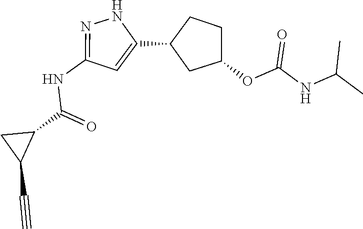 [SMILES: C#C[C@H]1C[C@@H]1C(=O)Nc1cc([C@@H]2CC[C@H](OC(=O)NC(C)C)C2)[nH]n1] | 345.2 | ¹HNMR (400 MHz, DMSO-d₆, ppm): δ 12.10 (brs, 1H), 10.72 (brs, 1H), 6.93 (d, J = 7.2 Hz, 1H), 6.26 (s, 1H), 5.05-4.96 (m, 1H), 3.62-3.50 (m, 1H), 3.09-2.98 (m, 1H), 2.81 (d, J = 2.0 Hz, 1H), 2.50-2.41 (m, 1H), 2.18-2.14 (m, 1H), 2.03-1.80 (m, 2H), 1.78-1.50 (m, 4H), 1.19-1.15 (m, 1H), 1.10-0.97 (m, 7H). |
| 146 | 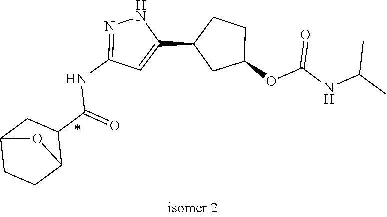 [SMILES: CC(C)NC(=O)O[C@@H]1CC[C@H](c2cc(NC(=O)C3CC4CCC3O4)n[nH]2)C1] | 377.3 | ¹HNMR (400 MHz, METHANOL-d₄, ppm): δ 6.37 (s, 1H), 5.15-5.08 (m, 1H), 4.72-4.63 (m, 2H), 3.75-3.67 (m, 1H), 3.19-3.10 (m, 1H), 2.77-2.72 (m, 1H), 2.54-2.46 (m, 1H), 2.15-2.04 (m, 2H), 1.99-1.53 (m, 9H), 1.15-1.05 (m, 6H). Chiral purity 99.62% de, retention time: 5.132 min. Chiral SFC analysis was performed on a Waters UPCC (CA-060), DAICELCHIRALCEL ®OD, 250*4.6 mm 5 μm, column heated to 35° C., eluted with a mobile phase of |
| | | | CO₂ and 15-40% MeOH (0.1% DEA), |
| | | | flowing at 3.0 mL/min. |
| 147 | 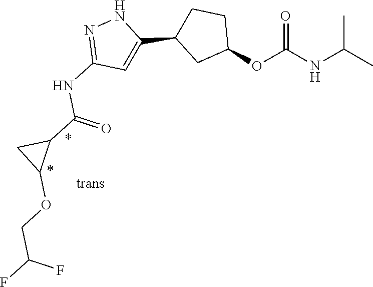 [SMILES: CC(C)NC(=O)O[C@@H]1CC[C@H](c2cc(NC(=O)C3CC3OCC(F)F)n[nH]2)C1] | 401.3 | ¹H NMR (400 MHz, DMSO-d₆, ppm): δ 12.05 (s, 1H), 10.59 (s, 1H), 6.95 (d, J = 8.0 Hz, 1H), 6.33-6.01 (m, 2H), 5.05-4.89 (m, 1H), 3.83-3.71 (m, 2H), 3.65-3.50 (m, 2H), 3.08-2.95 (m, 1H), 2.48-2.38 (m, 1H), 2.09- 1.94 (m, 2H), 1.93-1.81 (m, 1H), 1.71- 1.62 (m, 2H), 1.60-1.50 (m, 1H), 1.18-1.06 (m, 2H), 1.02 (d, J = 8.0 Hz, 6H). |
| 148 | 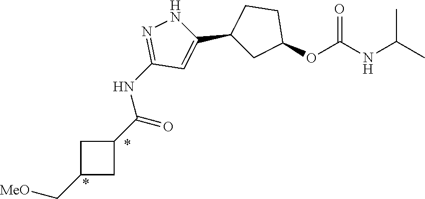 [SMILES: COCC1CC(C(=O)Nc2cc([C@H]3CC[C@@H](OC(=O)NC(C)C)C3)[nH]n2)C1] | 379.4 | ¹H NMR (400 MHz, METHANOL-d₄, ppm): δ 6.38 (s, 1H), 5.08 (s, 1H), 3.75-3.63 (m, 1H), 3.37 (d, J = 8.0 Hz, 2H), 3.30 (s, 3H), 3.20-3.08 (m, 2H), 2.57-2.44 (m, 2H), 2.31-2.21 (m, 2H), 2.14-2.00 (m, 3H), 1.98-1.71 (m, 4H), 1.15-1.06 (m, 6H). Chiral purity 100% de, retention time: 3.165 min. Chiral SFC analysis was performed on a DAICELCHIRALCEL ®OD, 250*4.6 mm 5 μm, column heated to 35° C., eluted with a mobile phase of CO₂ |
| | | | and 15-40% MeOH (0.1% DEA), |
| | | | flowing at 1.5 mL/min. |
| 149 | 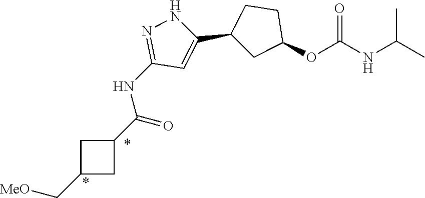 [SMILES: COCC1CC(C(=O)Nc2cc([C@H]3CC[C@@H](OC(=O)NC(C)C)C3)[nH]n2)C1] | 379.4 | ¹H NMR (400 MHz, METHANOL-d₄, ppm): δ 6.39 (s, 1H), 5.08 (s, 1H), 3.75-3.61 (m, 1H), 3.46 (d, J = 8.0 Hz, 2H), 3.36 (s, 3H), 3.28-3.10 (m, 2H), 2.63-2.45 (m, 2H), 2.44-2.35 (m, 2H), 2.15-1.99 (m, 3H), 1.97-1.72 (m, 4H), 1.16-1.04 (m, 6H). Chiral purity 100% de, retention time: 3.253 min. Chiral SFC analysis was performed on a DAICELCHIRAL ®OD, 250*4.6 mm 5 μm, column heated to 35° C., eluted with a mobile phase of CO₂ and |
| | | | 15-40% MeOH (0.1% DEA), flowing |
| | | | at 1.5 mL/min. |
| 150 | 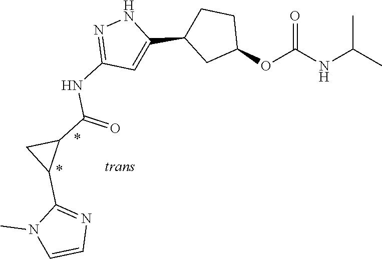 [SMILES: CC(C)NC(=O)O[C@@H]1CC[C@H](c2cc(NC(=O)C3CC3c3nccn3C)n[nH]2)C1] | 401.2 | ¹HNMR (300 MHz, METHANOL-d₄, ppm): δ 7.03 (d, J = 1.2 Hz, 1H), 6.85 (d, J = 1.5 Hz, 1H), 6.35 (s, 1H), 5.12-5.02 (m, 1H), 3.75-3.62 (m, 4H), 3.20-3.08 (m, 1H), 2.55-2.42 (m, 2H), 2.20-1.70 (m, 6H), 1.62-1.45 (m, 2H), 1.11 (d, J = 6.6 Hz, 6H). |
| | It's obtained from the | | |
| | intermediate 150-1. | | |
| 151 | 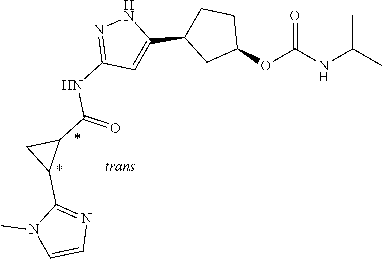 [SMILES: CC(C)NC(=O)O[C@@H]1CC[C@H](c2cc(NC(=O)C3CC3c3nccn3C)n[nH]2)C1] | 401.2 | ¹H NMR (300 MHz, DMSO-d₆, ppm): δ 12.10 (brs, 1H), 10.65 (brs, 1H), 7.03 (s, 1H), 6.94 (d, J = 7.5 Hz, 1H), 6.71 (s, 1H), 6.31 (s, 1H), 5.03-4.90 (m, 1H), 3.62 (s, 3H), 3.60-3.50 (m, 1H), 3.10-2.97 (m, 1H), 2.47-2.19 (m, 3H), 2.05-1.80 (m, 2H), 1.78-1.50 (m, 3H), 1.42-1.29 (m, 2H), 1.03 (d, J = 6.3 Hz, 6H). |
| | It's obtained from the | | |
| | intermediate 150-2. | | |
| 152 | 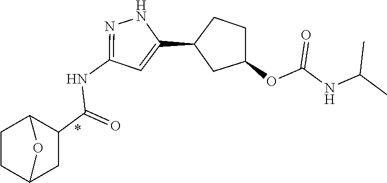 [SMILES: CC(C)NC(=O)O[C@@H]1CC[C@H](c2cc(NC(=O)C3CC4CCC3O4)n[nH]2)C1] | 377.0 | ¹HNMR (400 MHz, METHANOL-d₄, ppm): δ 6.34 (s, 1H), 5.15-5.08 (m, 1H), 4.71-4.65 (m, 2H), 3.74-3.67 (m, 1H), 3.19-3.12 (m, 1H), 2.77-2.72 (m, 1H), 2.53-2.46 (m, 1H), 2.13-2.04 (m, 2H), 1.99-1.50 (m, 9H), 1.15-1.05 (m, 6H). Chiral purity 99.94% de, retention time: 4.447 min. Chiral SFC analysis was performed on a Waters UPCC (CA-060), column: DAICELCHIRALCEL ®OD, |
| | | | 250*4.6 mm, 5 μm, column heated to |
| | | | 35° C., eluted with a mobile phase of |
| | | | CO₂ and 15-40% MeOH (0.1% DEA), |
| | | | flowing at 3.0 mL/min. |
| 153 | 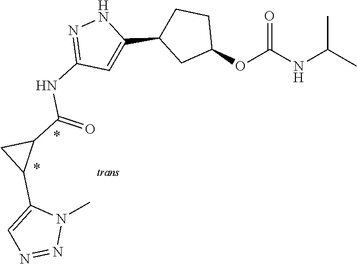 [SMILES: CC(C)NC(=O)O[C@@H]1CC[C@H](c2cc(NC(=O)C3CC3c3cnnn3C)n[nH]2)C1] | 402.4 | ¹H NMR (300 MHz, METHANOL-d₄, ppm): δ 7.46 (s, 1H), 6.37 (s, 1H), 5.15-5.05 (m, 1H), 4.08 (s, 3H), 3.75-3.62 (m, 1H), 3.21-3.09 (m, 1H), 2.56-2.39 (m, 2H), 2.15-2.05 (m, 2H). 1.99-1.72 (m, 4H), 1.69-1.61 (m, 1H), 1.39-1.32 (m, 1H), 1.15-1.05 (m, 6H). Chiral purity 100.0% de, retention time: 3.511 min. Chiral SFC analysis was performed on a Waters UPCC (CA-060), column: DIACELCHIRALPAK ®IG, 100*3.0 mm 3.0 μm column heated to 35° C., eluted with a mobile phase of CO₂ and 40% MeOH (0.1% DEA), flowing at 1.5 mL/min. |
| 154 | 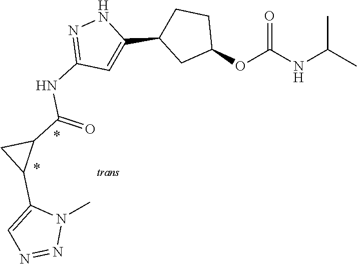 [SMILES: CC(C)NC(=O)O[C@@H]1CC[C@H](c2cc(NC(=O)C3CC3c3cnnn3C)n[nH]2)C1] | 402.2 | ¹H NMR (300 MHz, METHANOL-d₄, ppm): δ 7.46 (s, 1H), 6.37 (s, 1H), 5.15-5.05 (m, 1H), 4.08 (s, 3H), 3.75-3.62 (m, 1H), 3.20-3.09 (m, 1H), 2.58-2.41 (m, 2H), 2.15-2.05 (m, 2H), 1.99-1.72 (m, 4H), 1.69-1.61 (m, 1H), 1.39-1.32 (m, 1H), 1.15-1.05 (m, 6H). Chiral purity 100.0% de, retention time: 4.002 min. Chiral SFC analytical method: Waters UPCC (CA-060), column: DAICELCHIRALPAK ®IG, 100*3.0 mm 3.0 μm column heated to 35° C., eluted with a mobile phase of CO₂ and 40% MeOH (0.1% DEA), flowing at 1.5 mL/min. |
| 155 | 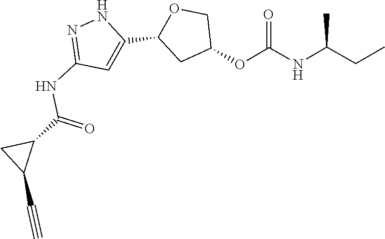 [SMILES: C#C[C@H]1C[C@@H]1C(=O)Nc1cc([C@H]2C[C@@H](OC(=O)N[C@@H](C)CC)CO2)[nH]n1] | 361.2 | ¹H NMR (400 MHz, METHANOL-d₄, ppm): δ 6.45 (s, 1H), 5.23-5.20 (m, 1H), 4.97-4.90 (m, 1H), 4.03-4.00 (m, 1H), 3.95-3.91 (m, 1H), 3.52-3.47 (m, 1H), 2.71-2.61 (m, 1H), 2.24 (d, J = 2.0 Hz, 1H), 2.15-2.03 (m, 2H), 1.78- 1.75 (m, 1H), 1.43-1.40 (m, 2H), 1.38-1.31 (m, 1H), 1.15-1.12 (m, 1H), 1.08 (d, J = 6.8 Hz, 3H), 0.92-0.81 (m, 3H). |
| 156 | 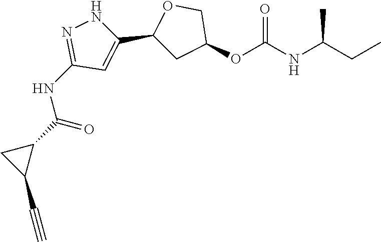 [SMILES: C#C[C@H]1C[C@@H]1C(=O)Nc1cc([C@@H]2C[C@H](OC(=O)N[C@@H](C)CC)CO2)[nH]n1] | 361.2 | ¹H NMR (400 MHz, DMSO-d₆, ppm): δ 12.34 (s, 1H), 10.79 (s, 1H), 7.02 (d, J = 8.0 Hz, 1H), 6.42 (s, 1H), 5.14 (s, 1H), 4.80 (s, 1H), 3.85 (s, 2H), 3.58-3.55 (m, 1H), 2.83 (s, 1H), 2.69- 2.62 (m, 1H), 2.18-2.15 (m, 1H), 1.88-1.80 (m, 1H), 1.66-1.60 (m, 1H), 1.38-1.32 (m, 2H), 1.23-0.72 (m, 8H). |
| 157 | 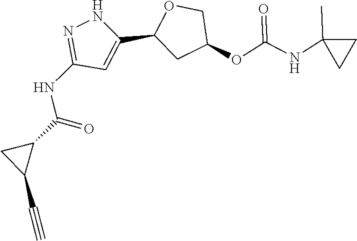 [SMILES: C#C[C@H]1C[C@@H]1C(=O)Nc1cc([C@@H]2C[C@H](OC(=O)NC3(C)CC3)CO2)[nH]n1] | 359.2 | ¹H NMR (400 MHz, DMSO-d₆, ppm): δ 12.33 (s, 1H), 10.80 (s, 1H), 7.50 (s, 1H), 6.40 (s, 1H), 5.14-5.10 (m, 1H), 4.80-4.77 (m, 1H), 3.86-3.80 (m, 2H), 2.83 (s, 1H), 2.69-2.62 (m, 1H), 2.18-2.15 (m, 1H), 1.88-1.80 (m, 1H), 1.66-1.60 (m, 1H), 1.23 (s, 3H), 1.21- 1.05 (m, 2H), 0.65-0.40 (m, 4H). |
| 158 | 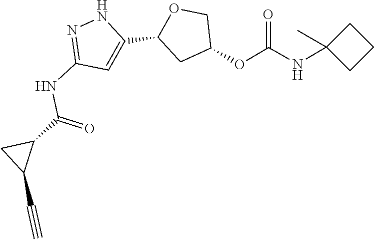 [SMILES: C#C[C@H]1C[C@@H]1C(=O)Nc1cc([C@H]2C[C@@H](OC(=O)NC3(C)CCC3)CO2)[nH]n1] | 373.2 | ¹H NMR (400 MHz, DMSO-d₆, ppm): δ 6.43 (s, 1H), 5.25-5.18 (m, 1H), 5.05-4.97 (m, 1H), 4.03-3.87 (m, 2H), 2.72-2.62 (m, 1H), 2.24-2.20 (m, 3H), 2.16-2.05 (m, 2H), 1.95-1.82 (m, 2H), 1.79-1.72 (m, 3H), 1.40-1.30 (m, 4H), 1.16-1.14 (m, 1H). |
| 159 | 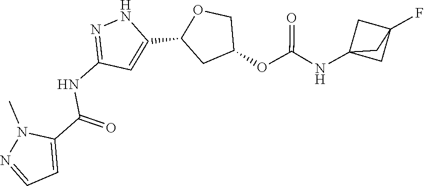 [SMILES: Cn1nccc1C(=O)Nc1cc([C@H]2C[C@@H](OC(=O)NC34CC(F)(C3)C4)CO2)[nH]n1] | 405.2 | ¹H NMR (400 MHz, DMSO-d₆, ppm): δ 7.51 (d, J = 2.0 Hz, 1H), 6.94 (d, J = 1.6 Hz, 1H), 6.52 (s, 1H), 5.24 (s, 1H), 5.01 (s, 1H), 4.16 (s, 3H), 4.05-3.96 (m, 2H), 2.76-2.69 (m, 1H), 2.27-2.12 (m, 7H). |
| 160 | 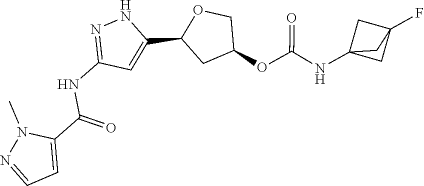 [SMILES: Cn1nccc1C(=O)Nc1cc([C@@H]2C[C@H](OC(=O)NC34CC(F)(C3)C4)CO2)[nH]n1] | 405.2 | ¹H NMR (400 MHz, DMSO-d₆, ppm): δ 10.82 (s, 1H), 8.11 (s, 1H), 7.49 (d, J = 2.0 Hz, 1H), 7.15 (d, J = 2.0 Hz, 1H), 6.53 (s, 1H) 5.20-5.18 (m, 1H), 4.85-4.80 (m, 1H), 4.08 (s, 3H), 3.90-3.85 (m, 2H), 2.76-2.65 (m, 2H), 2.28-1.95 (m, 6H). |
| 161 | 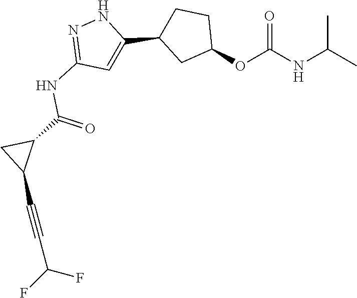 [SMILES: CC(C)NC(=O)O[C@@H]1CC[C@H](c2cc(NC(=O)[C@H]3C[C@@H]3C#CC(F)F)n[nH]2)C1] | 395.1 | ¹H NMR (400 MHz, DMSO-d₆): δ 12.10 (brs, 1H), 10.78 (brs, 1H), 6.94 (d, J = 7.6 Hz, 1H), 6.83-6.54 (m, 1H), 6.27 (s, 1H), 5.05-4.93 (m, 1H), 3.62-3.50 (m, 1H), 3.09-2.98 (m, 1H), 2.50-2.41 (m, 1H), 2.32-2.28 (m, 1H), 2.03-1.50 (m, 6H), 1.32-1.23 (m, 2H), 1.02 (d, J = 6.4 Hz, 6H). |
| 162 | 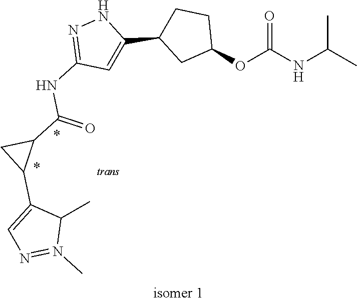 | 415.4 | ¹HNMR (400 MHz, METHANOL-d₄, ppm): δ 7.15 (s, 1H), 6.38 (s, 1H), 5.13-5.06 (m, 1H), 3.73 (s, 3H), 3.71-3.66 (m, 1H), 3.19-3.13 (m, 1H), 2.53-2.46 (m, 1H), 2.28 (s, 3H), 2.19-2.08 (m, 2H), 1.97-1.73 (m, 5H), 1.50-1.46 (m, 1H), 1.19-1.05 (m, 7H). Chiral purity 100.0% de, retention time: 4.766 min. Chiral SFC analysis was performed on a Waters UPCC (CA-060), DAICELCHIRALCEL ®OZ, 100*3.0 mm 3.0 μm, column heated to 35° C., eluted with a mobile phase of CO₂ and 5.0-40% MeOH (0.1% DEA), flowing at 1.5 mL/min. |
| 163 | 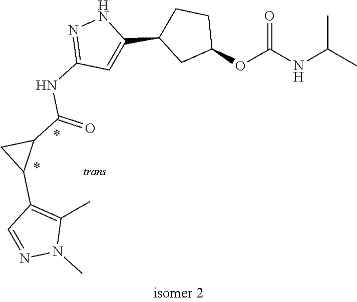 [SMILES: Cc1c(C2CC2C(=O)Nc2cc([C@H]3CC[C@@H](OC(=O)NC(C)C)C3)[nH]n2)cnn1C] | 415.4 | ¹HNMR (400 MHz, METHANOL-d₄, ppm): δ 7.15 (s, 1H), 6.38 (s, 1H), 5.13-5.06 (m, 1H), 3.73 (s, 3H), 3.71-3.66 (m, 1H), 3.19-3.14 (m, 1H), 2.54-2.45 (m, 1H), 2.29 (s, 3H), 2.19-2.10 (m, 2H), 1.94-1.77 (m, 5H), 1.49-1.46 (m, 1H), 1.19-1.05 (m, 7H). Chiral purity 100.0% de, retention time: 5.603 min. Chiral SFC analysis was performed on a Waters UPCC (CA-060), DAICELCHIRALCEL ®OZ, 100*3.0 mm 3.0 μm, column heated to 35° C., eluted with a mobile phase of CO₂ and 5.0-40% MeOH (0.1% DEA), flowing at 1.5 mL/min. |
| 164 | 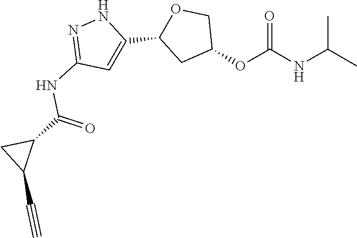 [SMILES: C#C[C@H]1C[C@@H]1C(=O)Nc1cc([C@H]2C[C@@H](OC(=O)NC(C)C)CO2)[nH]n1] | 347.2 | ¹H NMR (400 MHz, METHANOL-d₄, ppm): δ 6.42 (s, 1H), 5.25-5.20 (m, 1H), 4.97-4.92 (m, 1H), 4.02-3.98 (m, 1H), 3.93-3.85 (m, 1H), 3.73-3.65 (m, 1H), 2.72-2.63 (m, 1H), 2.24 (d, J = 2.0 Hz, 1H), 2.11-2.06 (m, 2H), 1.86-1.77 (m, 1H), 1.37-1.32 (m, 1H), 1.19-1.14 (m, 1H), 1.12-1.10 (m, 6H). |
| 165 | 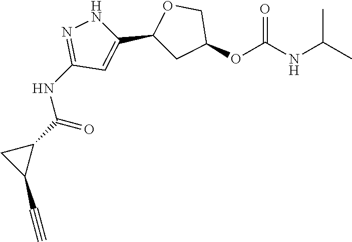 [SMILES: C#C[C@H]1C[C@@H]1C(=O)Nc1cc([C@@H]2C[C@H](OC(=O)NC(C)C)CO2)[nH]n1] | 347.2 | ¹H NMR (400 MHz, DMSO-d₆, ppm): δ 12.34 (s, 1H), 10.79 (s, 1H), 7.09 (d, J = 8.0 Hz, 1H), 6.42 (s, 1H), 5.14 (s, 1H), 4.80 (s, 1H), 3.85 (s, 2H), 3.58-3.55 (m, 1H), 2.83 (s, 1H), 2.69- 2.62 (m, 1H), 2.18-2.15 (m, 1H), 1.88-1.80 (m, 1H), 1.66-1.60 (m, 1H), 1.19-1.05 (m, 2H), 1.03-0.99 (m, 6H). |
| 166 | 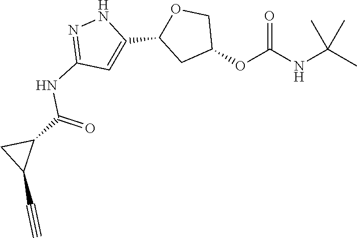 [SMILES: C#C[C@H]1C[C@@H]1C(=O)Nc1cc([C@H]2C[C@@H](OC(=O)NC(C)(C)C)CO2)[nH]n1] | 361.2 | ¹H NMR (400 MHz, METHANOL-d₄, ppm): δ 6.44 (s, 1H), 5.17-5.10 (m, 1H), 5.02-4.96 (m, 1H), 4.02-3.99 (m, 1H), 3.93-3.89 (m, 1H), 2.72-2.64 (m, 1H), 2.24 (d, J = 2.4 Hz, 1H), 2.13-2.01 (m, 2H), 1.81-1.76 (m, 1H), 1.40-1.31 (m, 1H), 1.30 (s, 9H), 1.19-1.12 (m, 1H). |
| 167 | 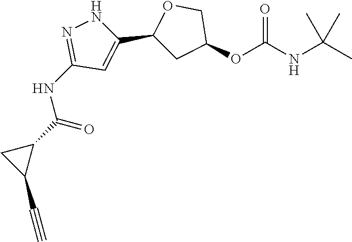 [SMILES: C#C[C@H]1C[C@@H]1C(=O)Nc1cc([C@@H]2C[C@H](OC(=O)NC(C)(C)C)CO2)[nH]n1] | 361.2 | ¹H NMR (400 MHz, DMSO-d₆, ppm): δ 12.32 (s, 1H), 10.80 (s, 1H), 6.92 (s, 1H), 6.42 (s, 1H), 5.12 (s, 1H), 4.80 (s, 1H), 3.81-3.78 (m, 2H), 3.38-33 (m, 1H), 2.83 (s, 1H), 2.69-2.62 (m, 1H), 2.18-2.15 (m, 1H), 1.88-1.80 (m, 1H), 1.66-1.60 (m, 1H), 1.20 (s, 9H), 1.13- 1.09 (m, 1H). |
| 168 | 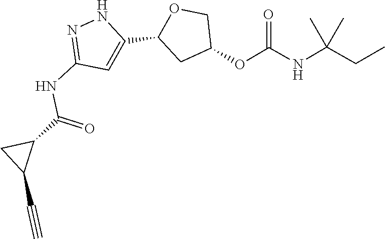 [SMILES: C#C[C@H]1C[C@@H]1C(=O)Nc1cc([C@H]2C[C@@H](OC(=O)NC(C)(C)CC)CO2)[nH]n1] | 375.2 | ¹H NMR (400 MHz, METHANOL-d₄, ppm): δ 6.45 (s, 1H), 5.16 (s, 1H), 4.97 (s, 1H), 4.02-3.99 (m, 1H), 3.93-3.89 (m, 1H), 2.75-2.62 (m, 1H), 2.24 (d, J = 2.4 Hz, 1H), 2.09-2.06 (m, 2H), 1.83-1.73 (m, 1H), 1.70-1.55 (m, 2H), 1.41-1.29 (m, 1H), 1.24-1.10 (m, 7H), 0.81 (t, J = 7.6 Hz, 3H). |
| 169 | 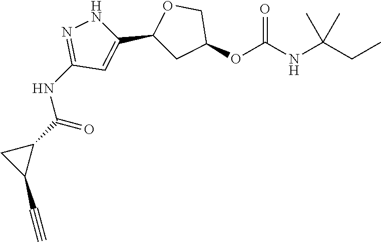 [SMILES: C#C[C@H]1C[C@@H]1C(=O)Nc1cc([C@@H]2C[C@H](OC(=O)NC(C)(C)CC)CO2)[nH]n1] | 375.2 | ¹H NMR (400 MHz, DMSO-d₆, ppm): δ 12.34 (s, 1H), 10.80 (s, 1H), 6.78 (s, 1H), 6.42 (s, 1H), 5.14-5.10 (m, 1H), 4.80-4.77 (m, 1H), 3.82 (s, 2H), 3.38-3.35 (m, 1H), 2.83 (s, 1H), 2.69- 2.62 (m, 1H), 2.18-2.15 (m, 1H), 1.88-1.80 (m, 1H), 1.66-1.60 (m, 1H), 1.58-1.52 (m, 2H), 1.23-1.08 (m, 7H), 0.74 (t, J = 7.2 Hz, 3H). |
| 170 | 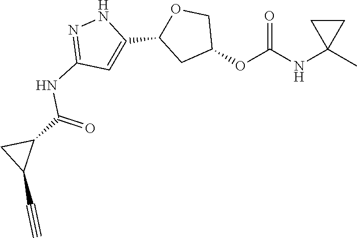 [SMILES: C#C[C@H]1C[C@@H]1C(=O)Nc1cc([C@H]2C[C@@H](OC(=O)NC3(C)CC3)CO2)[nH]n1] | 359.2 | ¹H NMR (400 MHz, METHANOL-d₄, ppm): δ 6.39 (s, 1H), 5.25-5.20 (m, 1H), 4.95-4.90 (m, 1H), 4.10-3.87 (m, 2H), 2.75-2.62 (m, 1H), 2.24 (d, J = 2.0 Hz, 1H), 2.13-2.08 (m, 2H), 1.78-1.75 (m, 1H), 1.40-1.31 (m, 1H), 1.29 (s, 3H), 1.16-1.12 (m, 1H), 0.68-0.65 (m, 2H), 0.53-0.51 (m, 2H). |
| 171 | 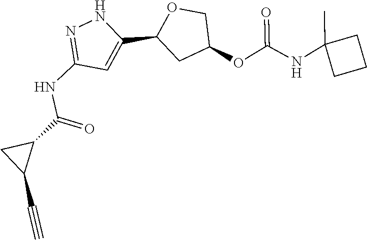 [SMILES: C#C[C@H]1C[C@@H]1C(=O)Nc1cc([C@@H]2C[C@H](OC(=O)NC3(C)CCC3)CO2)[nH]n1] | 373.2 | ¹H NMR (400 MHz, DMSO-d₆, ppm): δ 12.33 (s, 1H), 10.80 (s, 1H), 7.32 (s, 1H), 6.42 (s, 1H), 5.14-5.10 (m, 1H), 4.80-4.77 (m, 1H), 3.86-3.80 (m, 2H), 2.83 (s, 1H), 2.69-2.62 (m, 1H), 2.28-2.15 (m, 3H), 1.88-1.65 (m, 6H), 1.23 (s, 3H), 1.21-1.15 (m, 1H), 1.12-1.03 (m, 1H). |
| 172 | 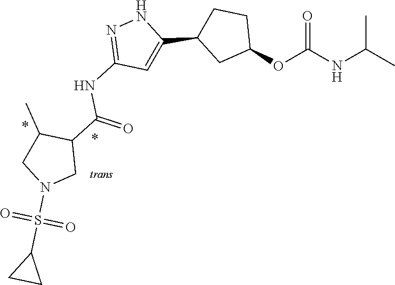 [SMILES: CC(C)NC(=O)O[C@@H]1CC[C@H](c2cc(NC(=O)C3CN(S(=O)(=O)C4CC4)CC3C)n[nH]2)C1] | 468.2 | ¹HNMR (400 MHz, DMSO-d₆, ppm): δ 12.10 (brs, 1H), 10.53 (s, 1H), 6.95-6.92 (m, 1H), 6.34 (s, 1H), 5.04-4.97 (m, 1H), 3.65-3.50 (m, 3H), 3.40-3.36 (m, 1H), 3.10-3.01 (m, 1H), 2.95-2.88 (m, 1H), 2.86-2.78 (m, 1H), 2.75-2.65 (m, 1H), 2.49-2.40 (m, 2H), 2.03-1.85 (m, 2H), 1.80-1.53 (m, 2H), 1.09-0.90 (m, 13H). |
| 173 | 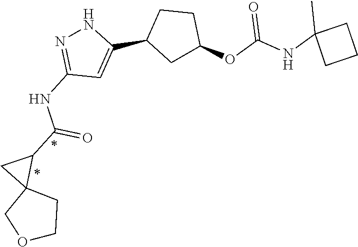 [SMILES: CC1(NC(=O)O[C@@H]2CC[C@H](c3cc(NC(=O)C4CC45CCOC5)n[nH]3)C2)CCC1] | 377.2 | ¹H NMR (400 MHz, DMSO-d₆, ppm): δ 12.00 (brs, 1H), 10.48 (brs, 1H), 6.94 (d, J = 7.2 Hz, 1H), 6.29 (s, 1H), 5.05-4.97 (m, 1H), 3.85-3.74 (m, 2H), 3.67-3.54 (m, 3H), 3.09-3.00 (m, 1H), 2.48-2.41 (m, 1H), 2.04-1.83 (m, 5H), 1.79-1.50 (m, 3H), 1.22-1.14 (m, 1H), 1.00-0.95 (m, 7H). |
| | It's obtained from the | | |
| | intermediate 173-1. | | |
| 174 | 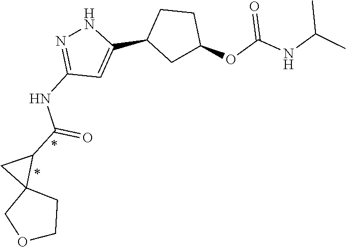 [SMILES: CC(C)NC(=O)O[C@@H]1CC[C@H](c2cc(NC(=O)C3CC34CCOC4)n[nH]2)C1] | 377.3 | ¹H NMR (400 MHz, DMSO-d₆, ppm): δ 12.00 (brs, 1H), 10.47 (brs, 1H), 6.94 (d, J = 7.2 Hz, 1H), 6.30 (s, 1H), 5.05-4.97 (m, 1H), 3.85-3.72 (m, 2H), 3.68-3.48 (m, 3H), 3.09-3.00 (m, 1H), 2.48-2.41 (m, 1H), 2.05-1.81 (m, 5H), 1.78-1.50 (m, 3H), 1.22-1.17 (m, 1H), 1.09-0.95 (m, 7H). |
| | It's obtained from the | | |
| | intermediate 173-2. | | |
| 175 | 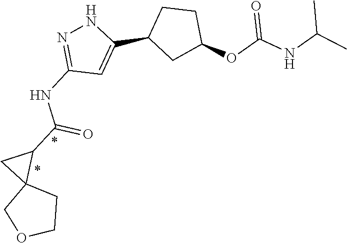 [SMILES: CC(C)NC(=O)O[C@@H]1CC[C@H](c2cc(NC(=O)C3CC34CCOC4)n[nH]2)C1] | 377.2 | ¹H NMR (400 MHz, DMSO-d₆, ppm): δ 12.01 (brs, 1H), 10.51 (brs, 1H), 6.94 (d, J = 7.6 Hz, 1H), 6.27 (s, 1H), 5.06-4.93 (m, 1H), 3.80-3.75 (m, 2H), 3.64-3.57 (m, 3H), 3.04-2.99 (m, 1H), 2.49-2.41 (m, 1H), 2.09-1.80 (m, 5H), 1.72-1.50 (m, 3H), 1.15-1.12 (m, 2H), 1.02 (d, J = 6.4 Hz, 6H). |
| | It's obtained from the | | |
| | intermediate 173-3. | | |
| 176 | 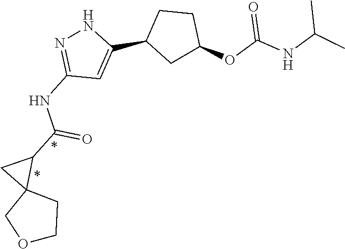 [SMILES: CC(C)NC(=O)O[C@@H]1CC[C@H](c2cc(NC(=O)C3CC34CCOC4)n[nH]2)C1] | 377.2 | ¹H NMR (400 MHz, DMSO-d₆, ppm): δ 12.02 (brs, 1H), 10.51 (brs, 1H), 6.94 (d, J = 7.6 Hz, 1H), 6.28 (s, 1H), 5.05-4.97 (m, 1H), 3.80-3.75 (m, 2H), 3.65-3.57 (m, 3H), 3.09-2.95 (m, 1H), 2.49-2.38 (m, 1H), 2.09-1.82 (m, 5H), 1.78-1.50 (m, 3H), 1.15-1.10 (m, 2H), 1.02 (d, J = 6.4 Hz, 6H). |
| | It's obtained from the | | |
| | intermediate 173-4. | | |
| 177 | 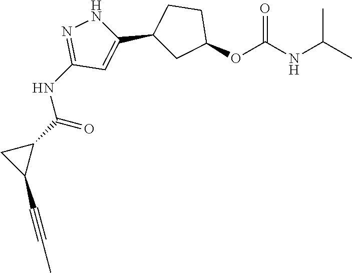 [SMILES: CC#C[C@H]1C[C@@H]1C(=O)Nc1cc([C@H]2CC[C@@H](OC(=O)NC(C)C)C2)[nH]n1] | 359.2 | ¹H NMR (400 MHz, METHANOL-d₄, ppm): δ 6.30 (s, 1H), 5.13-5.06 (m, 1H), 3.76-3.62 (m, 1H), 3.19-3.08 (m, 1H), 2.55-2.45 (m, 1H), 2.12-2.05 (m, 1H), 2.02-1.68 (m, 9H), 1.34-1.28 (m, 1H), 1.14-1.02 (m, 7H). |
| 178 | 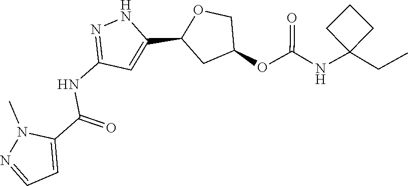 [SMILES: CCC1(NC(=O)O[C@@H]2CO[C@H](c3cc(NC(=O)c4ccnn4C)n[nH]3)C2)CCC1] | 403.3 | ¹H NMR (400 MHz, DMSO-d₆, ppm): δ 7.48 (d, J = 2.0 Hz, 1H), 7.27 (s, 1H), 7.16 (d, J = 2.0 Hz, 1H), 6.59 (s, 1H), 5.20-5.13 (m, 1H), 4.85-4.80 (m, 1H), 4.08 (s, 3H), 3.90-3.85 (m, 2H), 2.76-2.65 (m, 2H), 2.13-2.11 (m, 2H), 2.10-1.90 (m, 1H), 1.90-1.60 (m, 6H), 0.74 (t, J = 7.2 Hz, 3H). |
| 179 | 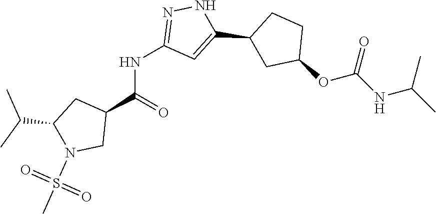 [SMILES: CC(C)NC(=O)O[C@@H]1CC[C@H](c2cc(NC(=O)[C@@H]3C[C@@H](C(C)C)N(S(C)(=O)=O)C3)n[nH]2)C1] | 470.3 | ¹H NMR (300 MHz, DMSO-d₆, ppm): δ 12.08 (brs, 1H), 10.47 (brs, 1H), 6.93 (d, J = 7.5 Hz, 1H), 6.31 (s, 1H), 5.07-4.92 (m, 1H), 3.68-3.55 (m, 2H), 3.50-3.46 (m, 2H), 3.28-3.20 (m, 1H), 3.09-2.98 (m, 1H), 2.90 (s, 3H), 2.12-1.82 (m, 6H), 1.74-1.55 (m, 3H), 1.03 (d, J = 6.6 Hz, 6H), 0.87 (d, J = 6.9 Hz, 6H). |
| 180 | 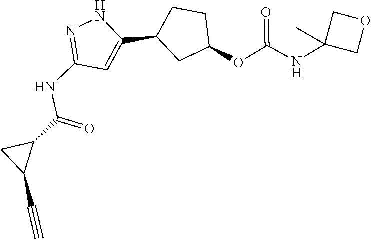 [SMILES: C#C[C@H]1C[C@@H]1C(=O)Nc1cc([C@H]2CC[C@@H](OC(=O)NC3(C)COC3)C2)[nH]n1] | 373.2 | ¹H NMR (300 MHz, METHANOL-d₄, ppm): δ 6.31 (s, 1H), 5.17-5.05 (m, 1H), 4.77-4.71 (m, 2H), 4.35 (d, J = 6.3 Hz, 2H), 3.21-3.01 (m, 1H), 2.58-2.45 (m, 1H), 2.23 (d, J = 2.1 Hz, 1H), 2.14-2.04 (m, 2H), 1.98-1.74 (m, 5H), 1.56 (s, 3H), 1.38-1.30 (m, 1H), 1.18-1.10 (m, 1H). |
| 181 | 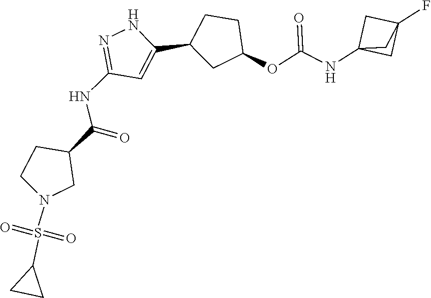 [SMILES: O=C(NC12CC(F)(C1)C2)O[C@@H]1CC[C@H](c2cc(NC(=O)[C@@H]3CCN(S(=O)(=O)C4CC4)C3)n[nH]2)C1] | 496.1 | ¹HNMR (400 MHz, METHANOL-d₄, ppm): δ 6.34 (s, 1H), 5.18-5.06 (m, 1H), 3.69-3.53 (m, 3H), 3.49-3.41 (m, 1H), 3.28-3.10 (m, 2H), 2.69-2.48 (m, 2H), 2.29-1.79 (m, 13H), 1.13-0.98 (m, 4H). |
| 182 | 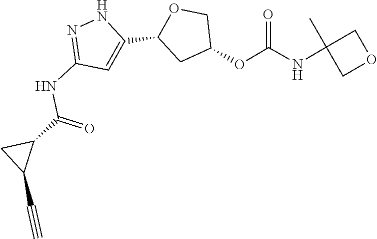 [SMILES: C#C[C@H]1C[C@@H]1C(=O)Nc1cc([C@H]2C[C@@H](OC(=O)NC3(C)COC3)CO2)[nH]n1] | 375.2 | ¹HNMR (400 MHz, METHANOL-d₄, ppm): δ 6.41 (s, 1H), 5.23 (s, 1H), 4.99 (s, 1H), 4.74 (s, 2H), 4.35 (s, 2H), 4.10- 3.87 (m, 2H), 2.69-2.63 (m, 1H), 2.24 (s, 1H), 2.20-2.03 (m, 2H), 1.89- 1.70 (m, 1H), 1.55 (s, 3H), 1.41- 1.28 (m, 2H), 1.16-1.10 (m, 1H). |
| 183 | 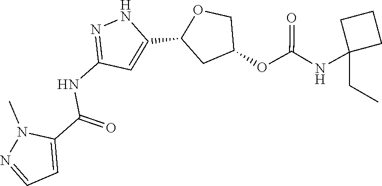 [SMILES: CCC1(NC(=O)O[C@H]2CO[C@@H](c3cc(NC(=O)c4ccnn4C)n[nH]3)C2)CCC1] | 403.3 | ¹HNMR (400 MHz, METHANOL-d₄, ppm): δ 7.51 (d, J = 2.0 Hz, 1H), 6.93 (d, J = 2.0 Hz, 1H), 6.59 (s, 1H), 5.20 (s, 1H), 5.03 (s, 1H), 4.16 (s, 3H), 4.05-4.01 (m, 1H), 4.00-3.86 (m, 1H), 2.77-2.60 (m, 1H), 2.16-2.10 (m, 3H), 1.97-1.90 (m, 2H), 1.86-1.60 (m, 4H), 0.80 (t, J = 7.2 Hz, 3H). |
| 184 | 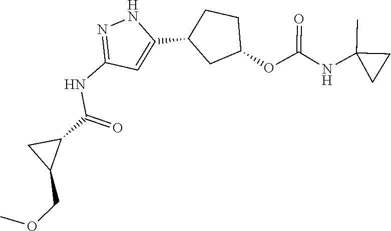 [SMILES: COC[C@H]1C[C@@H]1C(=O)Nc1cc([C@@H]2CC[C@H](OC(=O)NC3(C)CC3)C2)[nH]n1] | 377.2 | ¹HNMR (400 MHz, DMSO-d₆, ppm): δ 12.09 (brs, 1H), 10.49 (s, 1H), 7.40-7.30 (m, 1H), 6.24 (s, 1H), 5.08-4.93 (m, 1H), 3.37-3.34 (m, 1H), 3.32 (s, 4H), 3.23-3.13 (m, 1H), 3.10-2.95 (m, 1H), 2.50-2.42 (m, 1H), 1.99-1.81 (m, 2H), 1.79-1.38 (m, 5H), 1.22 (s, 3H), 0.96-0.92 (m, 1H), 0.75-0.72 (m, 1H), 0.60-0.55 (m, 2H), 0.50-0.42 (m, 2H). |
| 187 | 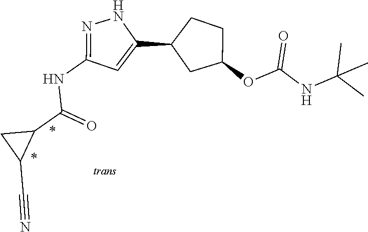 [SMILES: CC(C)(C)NC(=O)O[C@@H]1CC[C@H](c2cc(NC(=O)C3CC3C#N)n[nH]2)C1] | 360.2 | ¹H NMR (300 MHz, METHANOL-d₄, ppm): δ 6.48 (brs, 1H), 6.33 (s, 1H), 5.08-4.98 (m, 1H), 3.20-3.05 (m, 1H), 2.55-2.40 (m, 2H), 2.13-1.68 (m, 6H), 1.56-1.45 (m, 2H), 1.27 (s, 9H). Chiral purity 99.94% de; retention time: 4.739 min. Chiral SFC analysis was performed on a Waters UPCC, DAICEL CHIRALPAK ®OD-3, 100*3.0 mm 3 μm, column heated to 35° C., eluted with a mobile phase of CO₂ and 20% MeOH, flowing at 2.0 mL/min. |
| 188 | 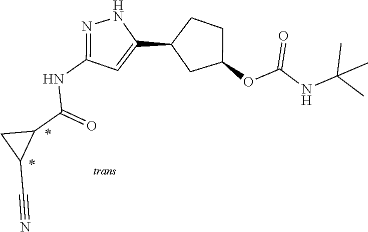 [SMILES: CC(C)(C)NC(=O)O[C@@H]1CC[C@H](c2cc(NC(=O)C3CC3C#N)n[nH]2)C1] | 360.2 | ¹H NMR (300 MHz, DMSO-d₆, ppm): δ 12.12 (brs, 1H), 10.89 (brs, 1H), 6.74 (brs, 1H), 6.27 (s, 1H), 5.10-4.90 (m, 1H), 3.10-2.94 (m, 1H), 2.53-2.38 (m, 2H), 2.11-1.79 (m, 3H), 1.75-1.46 (m, 4H), 1.38-1.29 (m, 1H), 1.20 (s, 9H). Chiral purity 99.94% de; retention time: 4.739 min. Chiral SFC analysis was performed on a Waters UPCC, DAICEL CHIRALPAK ®OD-3, 100*3.0 mm 3 μm, column heated to 35° C., eluted with a mobile phase of CO₂ and 20% MeOH, flowing at 2.0 mL/min. |
| 189 | 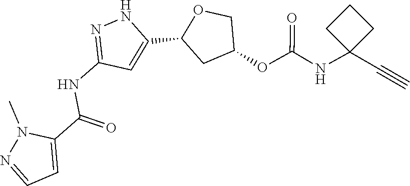 [SMILES: C#CC1(NC(=O)O[C@H]2CO[C@@H](c3cc(NC(=O)c4ccnn4C)n[nH]3)C2)CCC1] | 399.2 | ¹HNMR (400 MHz, METHANOL-d₄, ppm): δ 7.51 (d, J = 2.0 Hz, 1H), 6.95 (d, J = 2.0 Hz, 1H), 6.54 (s, 1H), 5.26 (s, 1H), 5.06-4.97 (m, 1H), 4.16 (s, 3H), 4.07-3.96 (m, 2H), 2.81-2.65 (m, 2H), 2.40-2.37 (m, 4H), 2.17-2.10 (m, 1H), 2.07-1.85 (m, 2H). |
| 190 | 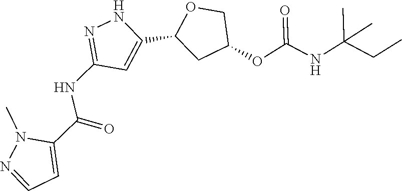 [SMILES: CCC(C)(C)NC(=O)O[C@H]1CO[C@@H](c2cc(NC(=O)c3ccnn3C)n[nH]2)C1] | 391.4 | ¹HNMR (400 MHz, METHANOL-d₄, ppm): δ 7.51 (d, J = 2.0 Hz, 1H), 6.94 (d, J = 2.0 Hz, 1H), 6.52 (s, 1H), 5.18 (s, 1H), 5.02 (s, 1H), 4.16 (s, 3H), 4.04-3.94 (m, 2H), 2.76-2.70 (m, 1H), 2.13-2.10 (m, 1H), 1.65-1.60 (m, 2H), 1.22 (s, 6H), 0.82 (t, J = 7.2 Hz, 3H). |
| 191 | 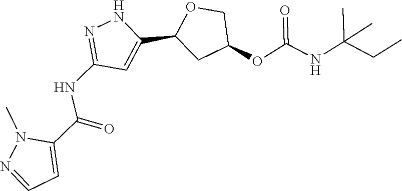 [SMILES: CCC(C)(C)NC(=O)O[C@@H]1CO[C@H](c2cc(NC(=O)c3ccnn3C)n[nH]2)C1] | 391.3 | ¹HNMR (400 MHz, DMSO-d₆, ppm): δ 12.51 (s, 1H), 10.82 (s, 1H), 7.49 (s, 1H), 7.16 (s, 1H), 6.81 (s, 1H), 6.59 (s, 1H), 5.20-5.13 (m, 1H), 4.85-4.80 (m, 1H), 4.08 (s, 3H), 3.90-3.85 (m, 2H), 2.70-2.65 (m, 1H), 1.91-1.80 (m, 1H), 1.60-1.54 (m, 2H), 1.20 (s, 6H), 0.74 (t, J = 7.2 Hz, 3H). |
| 192 | 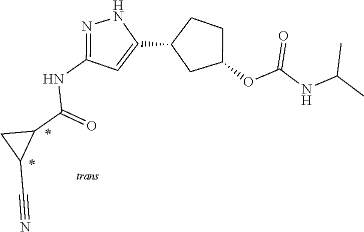 [SMILES: CC(C)NC(=O)O[C@H]1CC[C@@H](c2cc(NC(=O)C3CC3C#N)n[nH]2)C1] | 346.2 | ¹H NMR (400 MHz, DMSO-d₆, ppm): δ 12.14 (brs, 1H), 10.90 (brs, 1H), 6.93 (d, J = 7.2 Hz, 1H), 6.26 (s, 1H), 5.04-4.92 (m, 1H), 3.62-3.46 (m, 1H), 3.10-2.92 (m, 1H), 2.46-2.38 (m, 2H), 2.09-1.80 (m, 3H), 1.78-1.41 (m, 4H), 1.35-1.25 (m, 1H), 1.02 (d, J = 6.4 Hz, 6H). Chiral purity 99.94% de; retention time: 4.739 min. Chiral SFC analysis was performed on a Waters UPCC, DAICEL CHIRALPAK ®OD-3, 100*3.0 mm 3 μm column heated to 35° C., eluted with a mobile phase of CO₂ and 20% MeOH, flowing at 2.0 mL/min. |
| 193 | 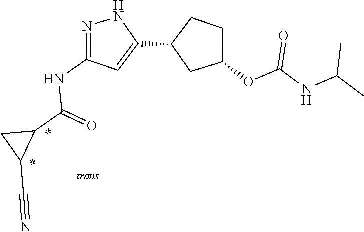 [SMILES: CC(C)NC(=O)O[C@H]1CC[C@@H](c2cc(NC(=O)C3CC3C#N)n[nH]2)C1] | 346.2 | ¹H NMR (400 MHz, DMSO-d₆, ppm): δ 12.12 (brs, 1H), 10.89 (brs, 1H), 6.93 (d, J = 7.2 Hz, 1H), 6.27 (s, 1H), 5.07-4.92 (m, 1H), 3.62-3.45 (m, 1H), 3.11-2.96 (m, 1H), 2.46-2.39 (m, 2H), 2.09-1.80 (m, 3H), 1.76-1.43 (m, 4H), 1.40-1.30 (m, 1H), 1.02 (d, J = 6.4 Hz, 6H). Chiral purity 99.94% de; retention time: 4.739 min. Chiral SFC analysis was performed on a Waters UPCC, DAICEL CHIRALPAK ®OD-3, 100*3.0 mm 3 μm column heated to 35° C., eluted with a mobile phase of CO₂ and 20% MeOH, flowing at 2.0 mL/min. |
| 194 | 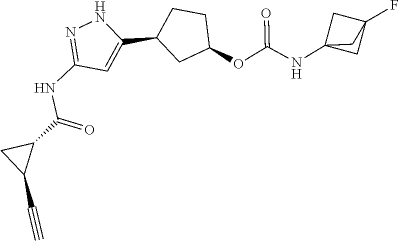 [SMILES: C#C[C@H]1C[C@@H]1C(=O)Nc1cc([C@H]2CC[C@@H](OC(=O)NC34CC(F)(C3)C4)C2)[nH]n1] | 387.1 | ¹HNMR (300 MHz, METHANOL-d₄, ppm): δ 6.30 (s, 1H), 5.21-5.02 (m, 1H), 3.18-3.13 (m, 1H), 2.55-2.46 (m, 1H), 2.26-2.23 (m, 7H), 2.13-2.07 (m, 2H), 2.00-1.70 (m, 5H), 1.38-1.28 (m, 1H), 1.18-1.12 (m, 1H). |
| 195 | 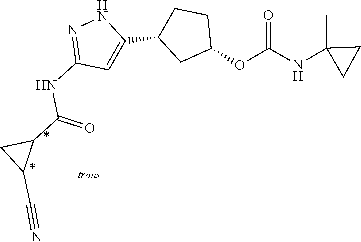 [SMILES: CC1(NC(=O)O[C@H]2CC[C@@H](c3cc(NC(=O)C4CC4C#N)n[nH]3)C2)CC1] | 358.2 | ¹H NMR (400 MHz, METHANOL-d₄, ppm): δ 6.29 (s, 1H), 5.15-5.06 (m, 1H), 3.25-3.12 (m, 1H), 2.59-2.42 (m, 2H), 2.20-2.04 (m, 2H), 1.96-1.72 (m, 4H), 1.56-1.51 (m, 2H), 1.30 (s, 3H), 0.75-0.65 (m, 2H), 0.57-0.53 (m, 2H). Chiral purity 99.80% de, retention time: 5.515 min. Chiral SFC analysis was performed on a Waters UPCC, OD-3, 250*4.6 mm, 3 μm, column heated to 35° C., eluted with a mobile phase of CO₂ and 20% MeOH, flowing at 2.0 mL/min. |
| 196 | 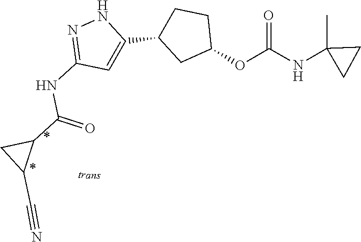 [SMILES: CC1(NC(=O)O[C@H]2CC[C@@H](c3cc(NC(=O)C4CC4C#N)n[nH]3)C2)CC1] | 358.1 | ¹H NMR (400 MHz, DMSO-d₆, ppm): δ 12.12 (brs, 1H), 10.89 (brs, 1H), 7.33-7.30 (m, 1H), 6.29 (s, 1H), 5.06-4.93 (m, 1H), 3.15-3.00 (m, 1H), 2.50-2.45 (m, 2H), 2.07-1.45 (m, 7H), 1.35-1.30 (m, 1H), 1.22 (s, 3H), 0.65-0.53 (m, 2H), 0.50-0.40 (m, 2H). Chiral purity 97.70% de, retention time: 7.080 min. Chiral SFC analysis was performed on a Waters UPCC, OD-3, 250*4.6 mm, 3 μm, column heated to 35° C., eluted with a mobile phase of CO₂ and 20% MeOH, flowing at 2.0 mL/min. |
| 197 | 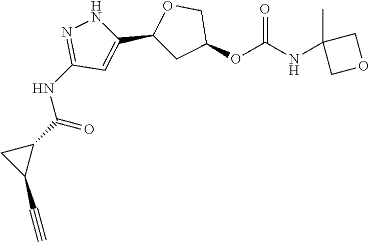 [SMILES: C#C[C@H]1C[C@@H]1C(=O)Nc1cc([C@@H]2C[C@H](OC(=O)NC3(C)COC3)CO2)[nH]n1] | 375.2 | ¹H NMR (400 MHz, DMSO-d₆): δ 10.81 (s, 1H), 7.77 (s, 1H), 6.39 (s, 1H), 5.16 (s, 1H), 4.80 (s, 1H), 4.56-4.50 (m, 2H), 4.25-4.20 (m, 2H), 3.87-3.80 (m, 2H), 2.83 (s, 1H), 2.70-2.60 (m, 1H), 2.20-2.12 (m, 1H), 1.90-1.80 (m, 1H), 1.70-1.61 (m, 1H), 1.46 (s, 3H), 1.21-1.18 (m, 1H), 1.12-1.10 (m, 1H). |
| 198 | 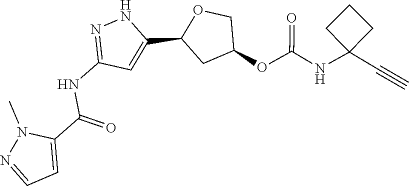 [SMILES: C#CC1(NC(=O)O[C@@H]2CO[C@H](c3cc(NC(=O)c4ccnn4C)n[nH]3)C2)CCC1] | 399.2 | ¹HNMR (400 MHz, DMSO-d₆, ppm): δ 12.54 (s, 1H), 10.82 (s, 1H), 7.91 (s, 1H), 7.49 (d, J = 1.6 Hz, 1H), 7.16 (d, J = 1.6 Hz, 1H), 6.59 (s, 1H), 5.20-5.13 (m, 1H), 4.85-4.80 (m, 1H), 4.08 (s, 3H), 3.90-3.85 (m, 2H), 3.20 (s, 1H), 2.80-2.71 (m, 1H), 2.30-2.20 (m, 4H), 1.91-1.70 (m, 3H). |
| 199 | 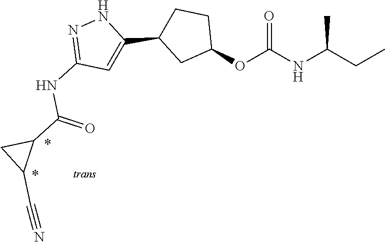 [SMILES: CC[C@H](C)NC(=O)O[C@@H]1CC[C@H](c2cc(NC(=O)C3CC3C#N)n[nH]2)C1] | 360.1 | ¹H NMR (400 MHz, DMSO-d₆, ppm): δ 12.13 (brs, 1H), 10.87 (brs, 1H), 6.88-6.86 (m, 1H), 6.27 (s, 1H), 5.04-4.98 (m, 1H), 3.36-3.31 (m, 1H), 3.03-2.92 (m, 1H), 2.50-2.43 (m, 2H), 2.03-1.86 (m, 3H), 1.78-1.50 (m, 4H), 1.45-1.28 (m, 3H), 0.99 (d, J = 6.8 Hz, 3H), 0.79 (t, J = 7.2 Hz, 3H). Chiral purity: 99.80% de; retention time: 2.925 min; Chiral SFC method: Waters UPCC, column: OD-3, 250 mm*4.6 mm, 3 μm column temperature: 35° C., MeOH in CO₂, 30%; flow rate: 2.0 mL/min. |
| 200 | 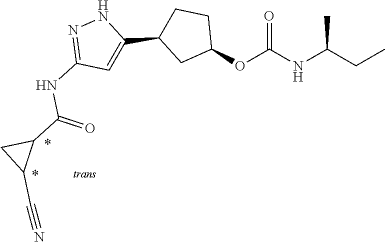 [SMILES: CC[C@H](C)NC(=O)O[C@@H]1CC[C@H](c2cc(NC(=O)C3CC3C#N)n[nH]2)C1] | 360.1 | ¹H NMR (300 MHz, DMSO-d₆, ppm): δ 12.15 (brs, 1H), 10.89 (brs, 1H), 6.85-6.83 (m, 1H), 6.26 (s, 1H), 5.05-4.97 (m, 1H), 3.45-3.31 (m, 1H), 3.12-2.96 (m, 1H), 2.52-2.42 (m, 2H), 2.08-1.82 (m, 3H), 1.78-1.49 (m, 4H), 1.36-1.29 (m, 3H), 0.99 (d, J = 6.8 Hz, 3H), 0.79 (t, J = 7.2 Hz, 3H). Chiral purity: 97.70% de; retention time: 3.504 min; Chiral SFC method: Waters UPCC, column: OD-3, 250 mm*4.6 mm, 3 μm, column temperature: 35° C., MeOH in CO₂, 30%; flow rate: 2.0 mL/min. |
| 201 | 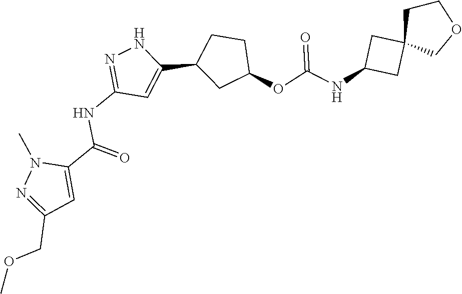 [SMILES: COCc1cc(C(=O)Nc2cc([C@H]3CC[C@@H](OC(=O)N[C@H]4C[C@@]5(CCOC5)C4)C3)[nH]n2)n(C)n1] | 473.2 | ¹H NMR (400 MHz, DMSO-d₆, ppm): δ 12.21 (brs, 1H), 10.73 (brs, 1H), 7.40 (d, J = 7.6 Hz, 1H), 7.11 (s, 1H), 6.40 (s, 1H), 5.10-4.93 (m, 1H), 4.33 (s, 2H), 4.05 (s, 3H), 3.91-3.80 (m, 1H), 3.67-3.50 (m, 4H), 3.27 (s, 3H), 3.18-3.01 (m, 1H), 2.28-2.10 (m, 2H), 2.09-1.55 (m, 10H). |
| 202 | 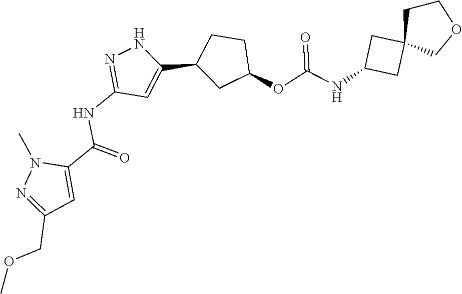 [SMILES: COCc1cc(C(=O)Nc2cc([C@H]3CC[C@@H](OC(=O)N[C@H]4C[C@]5(CCOC5)C4)C3)[nH]n2)n(C)n1] | 473.2 | ¹H NMR (400 MHz, DMSO-d₆, ppm): δ 12.22 (brs, 1H), 10.73 (brs, 1H), 7.37 (d, J = 8.0 Hz, 1H), 7.11 (s, 1H), 6.40 (s, 1H), 5.10-4.92 (m, 1H), 4.33 (s, 2H), 4.05 (s, 3H), 4.00-3.82 (m, 1H), 3.68-3.54 (m, 2H), 3.47 (s, 2H), 3.27 (s, 3H), 3.20-3.01 (m, 1H), 2.21-2.10 (m, 2H), 2.09-1.59 (m, 10H). |
| 203 | 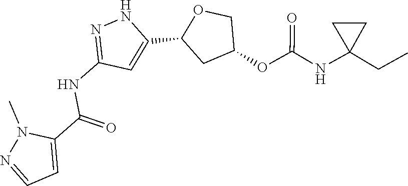 [SMILES: CCC1(NC(=O)O[C@H]2CO[C@@H](c3cc(NC(=O)c4ccnn4C)n[nH]3)C2)CC1] | 389.2 | ¹H NMR (400 MHz, METHANOL-d₄, ppm): δ 7.51-7.50 (m, 1H), 6.95-6.94 (m, 1H), 6.52 (s, 1H), 5.22 (s, 1H), 5.04-5.00 (m, 1H), 4.16 (s, 3H), 4.04-4.02 (m, 1H), 3.95-3.92 (m, 1H), 2.81-2.63 (m, 1H), 2.31-2.05 (m, 1H), 1.57-1.45 (m, 2H), 0.94-0.91 (m, 3H), 0.68 (t, J = 5.5 Hz, 2H), 0.56-0.54 (m, 2H). |
| 204 | 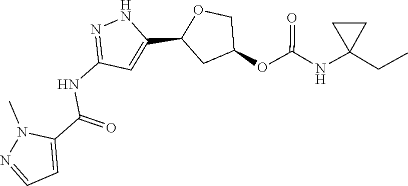 [SMILES: CCC1(NC(=O)O[C@@H]2CO[C@H](c3cc(NC(=O)c4ccnn4C)n[nH]3)C2)CC1] | 389.2 | ¹H NMR (400 MHz, DMSO-d₆, ppm): δ 12.51 (s, 1H), 10.81 (s, 1H), 7.51-7.49 (m, 2H), 7.16 (s, 1H), 6.57 (s, 1H), 5.15-5.13 (m, 1H), 4.85 (s, 1H), 4.09 (s, 3H), 3.84 (s, 2H), 2.75- 2.60 (m, 1H), 1.94-1.92 (m, 1H), 1.47- 1.41 (m, 2H), 0.86-0.82 (m, 3H), 0.63-0.41 (m, 4H). |
| 205 | 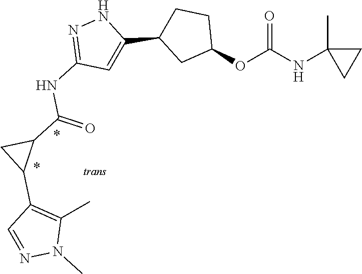 [SMILES: Cc1c(C2CC2C(=O)Nc2cc([C@H]3CC[C@@H](OC(=O)NC4(C)CC4)C3)[nH]n2)cnn1C] | 427.1 | ¹H NMR (300 MHz, METHANOL-d₄, ppm): δ 7.15 (s, 1H), 6.32 (s, 1H), 5.22-5.00 (m, 1H), 3.74 (s, 3H), 3.25-3.06 (m, 1H), 2.60-2.40 (m, 1H), 2.29 (s, 3H), 2.21-2.04 (m, 2H), 2.01-1.69 (m, 5H), 1.53-1.43 (m, 1H), 1.31 (s, 3H), 1.19-1.13 (m, 1H), 0.74-0.63 (m, 2H), 0.57-0.53 (m, 2H). Chiral purity: 97.32% de, retention time: 3.516 min. Chiral SFC method: Waters UPCC, column: CHIRALPAK OD-3 4.6 mm*250 mm, 3 μm, column temperature: 35° C., MeOH (0.1% DEA) in CO2, 0%-30.0%, flow rate: 2.0 mL/min. |
| 206 | 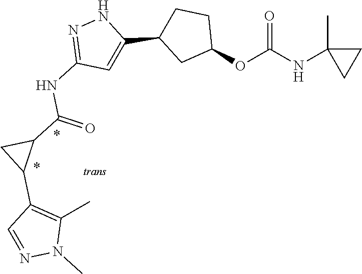 [SMILES: Cc1c(C2CC2C(=O)Nc2cc([C@H]3CC[C@@H](OC(=O)NC4(C)CC4)C3)[nH]n2)cnn1C] | 427.1 | ¹H NMR (300 MHz, METHANOL-d₄, ppm): δ 7.15 (s, 1H), 6.32 (s, 1H), 5.22-5.00 (m, 1H), 3.73 (s, 3H), 3.25-3.06 (m, 1H), 2.60-2.40 (m, 1H), 2.29 (s, 3H), 2.21-2.04 (m, 2H), 2.01-1.69 (m, 5H), 1.51-1.42 (m, 1H), 1.31 (s, 3H), 1.22-1.13 (m, 1H), 0.74-0.63 (m, 2H), 0.57-0.53 (m, 2H). Chiral purity: 98.70% de, retention time: 4.888 min. Chiral SFC method: Waters UPCC, column: CHIRALPAK OD-3 4.6 mm*250 mm, 3 μm, column temperature: 35° C., MeOH (0.1% DEA) in CO2, 0%-30.0%, flow rate: 2.0 mL/min. |
| 207 | 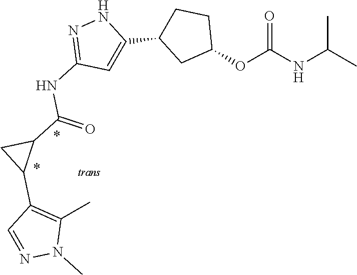 [SMILES: Cc1c(C2CC2C(=O)Nc2cc([C@@H]3CC[C@H](OC(=O)NC(C)C)C3)[nH]n2)cnn1C] | 415.2 | ¹H NMR (300 MHz, METHANOL-d₄, ppm): δ 7.15 (s, 1H), 6.34 (s, 1H), 5.20-5.01 (m, 1H), 3.75-3.65 (m, 4H), 3.21-3.06 (m, 1H), 2.55-2.45 (m, 1H), 2.29 (s, 3H), 2.21-2.04 (m, 2H), 2.02-1.71 (m, 5H), 1.51-1.45 (m, 1H), 1.19-1.10 (m, 7H). Chiral purity: 98.74% de, retention time: 3.196 min. Chiral SFC method: Waters UPCC, column: CHIRALPAK OD-3 4.6 mm*250 mm, 3 um, column temperature: 35° C., MeOH (0.1% DEA) in CO2, 0%-30.0%, flow rate: 2.0 mL/min. |
| 208 | 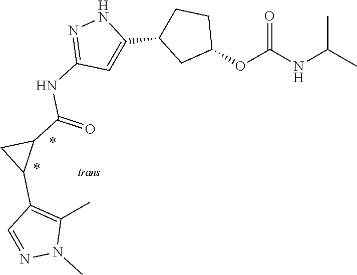 [SMILES: Cc1c(C2CC2C(=O)Nc2cc([C@@H]3CC[C@H](OC(=O)NC(C)C)C3)[nH]n2)cnn1C] | 415.2 | ¹H NMR (300 MHz, METHANOL-d₄, ppm): δ 7.15 (s, 1H), 6.34 (s, 1H), 5.20-5.01 (m, 1H), 3.80-3.60 (m, 4H), 3.21-3.06 (m, 1H), 2.55-2.45 (m, 1H), 2.29 (s, 3H), 2.21-2.04 (m, 2H), 2.02-1.71 (m, 5H), 1.51-1.45 (m, 1H), 1.22-1.05 (m, 7H). Chiral purity: 99.32% de, retention time: 4.908 min. Chiral SFC method: Waters UPCC, column: CHIRALPAK OD-3 4.6 mm*250 mm, 3 μm, column temperature: 35° C., MeOH (0.1% DEA) in CO2, 0%-30.0%, flow rate: 2.0 mL/min. |
| 209 | 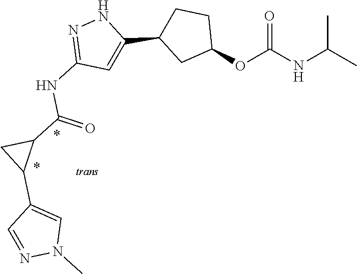 [SMILES: CC(C)NC(=O)O[C@@H]1CC[C@H](c2cc(NC(=O)C3CC3c3cnn(C)c3)n[nH]2)C1] | 401.2 | ¹H NMR (300 MHz, METHANOL-d₄, ppm): δ 7.45 (s, 1H), 7.32 (s, 1H), 6.32 (s, 1H), 5.14-5.01 (m, 1H), 3.82 (s, 3H), 3.75-3.62 (m, 1H), 3.19-3.08 (m, 1H), 2.55-2.42 (m, 1H), 2.32-2.21 (m, 1H), 2.12-2.00 (m, 1H), 1.98-1.69 (m, 5H), 1.52-1.42 (m, 1H), 1.20-1.13 (m, 1H), 1.10-1.02 (m, 6H). Chiral purity: 99.80% de, retention time: 3.325 min. Chiral SFC method: Waters UPCC, column: OD-3, 4.6 mm*250 mm, 3 μm, column temperature: 35° C., MeOH in CO2 30%, flow rate: 2.0 mL/min. |
| 210 | 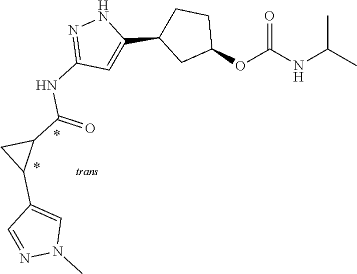 [SMILES: CC(C)NC(=O)O[C@@H]1CC[C@H](c2cc(NC(=O)C3CC3c3cnn(C)c3)n[nH]2)C1] | 401.2 | ¹H NMR (300 MHz, METHANOL-d₄, ppm): δ 7.45 (s, 1H), 7.32 (s, 1H), 6.32 (s, 1H), 5.13-5.01 (m, 1H), 3.82 (s, 3H), 3.75-3.62 (m, 1H), 3.19-3.08 (m, 1H), 2.55-2.42 (m, 1H), 2.32-2.21 (m, 1H), 2.12-2.00 (m, 1H), 1.98-1.69 (m, 5H), 1.52-1.42 (m, 1H), 1.20-1.05 (m, 7H). Chiral purity: 97.56% de, retention time: 5.002 min. Chiral SFC method: Waters UPCC, column: OD-3, 4.6 mm*250 mm, 3 μm, column temperature: 35° C., MeOH in CO₂, 30%, flow rate: 2.0 mL/min. |
| 211 | 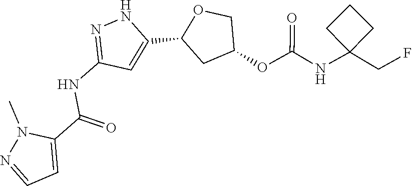 [SMILES: Cn1nccc1C(=O)Nc1cc([C@H]2C[C@@H](OC(=O)NC3(CF)CCC3)CO2)[nH]n1] | 407.2 | ¹H NMR (400 MHz, METHANOL-d₄, ppm): δ 7.51 (d, J = 2.0 Hz, 1H), 6.95 (d, J = 2.0 Hz, 1H), 6.53 (s, 1H), 5.23 (s, 1H), 4.98 (s, 1H), 4.53-4.2 (m, 2H), 4.16 (s, 3H), 4.06-4.04 (m, 1H), 3.98-3.93 (m, 1H), 2.82-2.62 (m, 1H), 2.18-2.13 (m, 5H), 1.90-1.78 (m, 2H). |
| 212 | 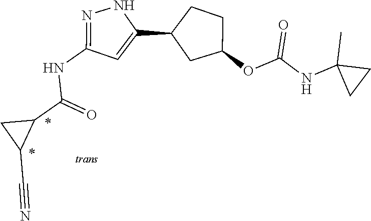 [SMILES: CC1(NC(=O)O[C@@H]2CC[C@H](c3cc(NC(=O)C4CC4C#N)n[nH]3)C2)CC1] | 358.1 | ¹H NMR (300 MHz, DMSO-d₆, ppm): δ 12.13 (s, 1H), 10.90 (s, 1H), 7.45-6.98 (m, 1H), 6.25 (s, 1H), 5.13-4.81 (m, 1H), 3.05-2.96 (m, 1H), 2.54-2.37 (m, 2H), 2.13-1.44 (m, 7H), 1.36-1.28 (m, 1H), 1.22 (s, 3H), 0.63-0.40 (m, 4H). Chiral purity: 99.74% de, retention time: 5.659 min. Chiral SFC method: Waters UPCC, column: CHIRALPAK OD-3 4.6 mm*250 mm, 3 μm, column temperature: 35° C., MeOH (0.1% DEA) in CO₂, 0%-20.0%, flow rate: 2.0 mL/min. |
| 213 | 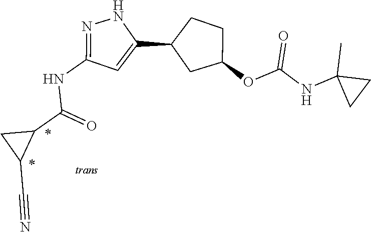 [SMILES: CC1(NC(=O)O[C@@H]2CC[C@H](c3cc(NC(=O)C4CC4C#N)n[nH]3)C2)CC1] | 358.1 | ¹H NMR (300 MHz, DMSO-d₆, ppm): δ 12.13 (s, 1H), 10.90 (s, 1H), 7.45-6.94 (m, 1H), 6.25 (s, 1H), 5.13-4.81 (m, 1H), 3.15-2.90 (m, 1H), 2.60-2.35 (m, 2H), 2.14-1.40 (m, 7H), 1.36-1.30 (m, 1H), 1.22 (s, 3H), 0.63-0.40 (m, 4H). Chiral purity: 97.08% de, retention time: 7.022 min. Chiral SFC method: Waters UPCC, column: CHIRALPAK OD-3 4.6 mm*250 mm, 3 μm, column temperature: 35° C., MeOH (0.1% DEA) in CO₂, 0%-20.0%, flow rate: 2.0 mL/min. |
| 214 | 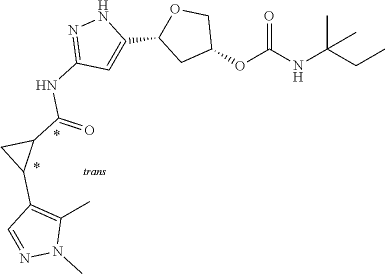 [SMILES: CCC(C)(C)NC(=O)O[C@H]1CO[C@@H](c2cc(NC(=O)C3CC3c3cnn(C)c3C)n[nH]2)C1] | 445.2 | ¹H NMR (400 MHz, METHANOL-d₄, ppm): δ 7.15 (s, 1H), 6.45 (s, 1H), 5.10 (s, 1H), 4.98 (s, 1H), 4.05-4.00 (m, 1H), 3.94-3.90 (m, 1H), 3.73 (s, 3H), 2.72-2.64 (m, 1H), 2.28 (s, 3H), 2.21-2.02 (m, 2H), 1.91-1.79 (m, 1H), 1.65-1.63 (m, 2H), 1.50-1.45 (m, 1H), 1.26-1.11 (m, 7H), 0.82 (t, J = 7.6 Hz, 3H). |
| 215 | 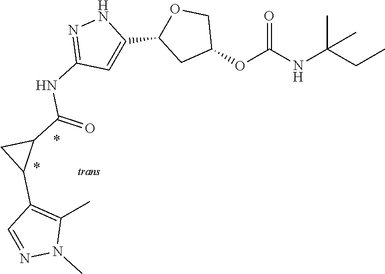 [SMILES: CCC(C)(C)NC(=O)O[C@H]1CO[C@@H](c2cc(NC(=O)C3CC3c3cnn(C)c3C)n[nH]2)C1] | 445.2 | ¹H NMR (400 MHz, METHANOL-d₄, ppm): δ 7.15 (s, 1H), 6.44 (s, 1H), 5.17 (s, 1H), 4.99-4.96 (m, 1H), 4.02-4.00 (m, 1H), 3.94-3.90 (m, 1H), 3.73 (s, 3H), 2.84-2.63 (m, 1H), 2.28 (s, 3H), 2.20-2.05 (m, 2H), 1.94- 1.82 (m, 1H), 1.65-1.63 (m, 2H), 1.50-1.46 (m, 1H), 1.24-1.13 (m, 7H), 0.81 (t, J = 7.2 Hz, 3H). |
| 216 | 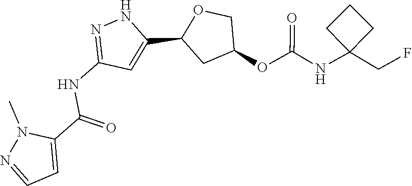 [SMILES: Cn1nccc1C(=O)Nc1cc([C@@H]2C[C@H](OC(=O)NC3(CF)CCC3)CO2)[nH]n1] | 407.2 | ¹H NMR (400 MHz, DMSO-d₆, ppm): δ 12.52 (s, 1H), 10.81 (s, 1H), 7.62 (s, 1H), 7.49 (s, 1H), 7.16 (s, 1H), 6.59 (s, 1H), 5.17 (s, 1H), 4.85 (s, 1H), 4.54 (s, 1H), 4.42 (s, 1H), 4.09 (s, 3H), 3.86-3.85 (m, 2H), 2.75-2.60 (m, 1H), 2.00 (s, 2H), 1.80-1.76 (m, 5H). |
| 217 | 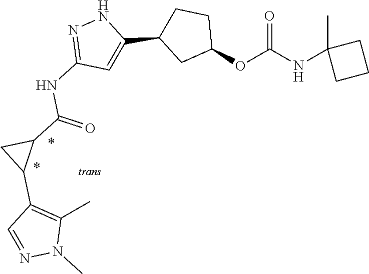 [SMILES: Cc1c(C2CC2C(=O)Nc2cc([C@H]3CC[C@@H](OC(=O)NC4(C)CCC4)C3)[nH]n2)cnn1C] | 441.2 | ¹H NMR (300 MHz, METHANOL-d₄, ppm): δ 7.15 (s, 1H), 6.35 (s, 1H), 5.22-5.00 (m, 1H), 3.74 (s, 3H), 3.23-3.10 (m, 1H), 2.60-2.42 (m, 1H), 2.29-2.04 (m, 7H), 2.00-1.69 (m, 9H), 1.53-1.42 (m, 1H), 1.39 (s, 3H), 1.22-1.10 (m, 1H). Chiral purity: 99.74% de, retention time: 4.153 min. Chiral SFC method: Waters UPCC, column: CHIRALPAK OD-3 4.6 mm*250 mm, 3 μm, column temperature: 35° C., MeOH (0.1% DEA) in CO₂, 0%-30.0%, flow rate: 2.0 mL/min. |
| 218 | 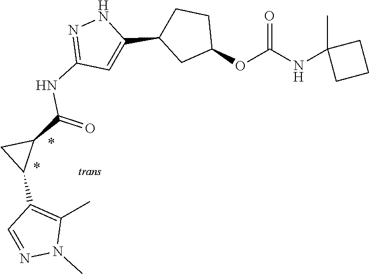 [SMILES: Cc1c([C@@H]2C[C@H]2C(=O)Nc2cc([C@H]3CC[C@@H](OC(=O)NC4(C)CCC4)C3)[nH]n2)cnn1C] | 441.2 | ¹H NMR (300 MHz, METHANOL-d₄, ppm): δ 7.15 (s, 1H), 6.35 (s, 1H), 5.22-5.00 (m, 1H), 3.74 (s, 3H), 3.21-3.10 (m, 1H), 2.56-2.42 (m, 1H), 2.36-2.04 (m, 7H), 2.00-1.69 (m, 9H), 1.53-1.42 (m, 1H), 1.39 (s, 3H), 1.21-1.13 (m, 1H). Chiral purity: 98.64% de, retention time: 5.754 min. Chiral SFC method: Waters UPCC, column: CHIRALPAK OD-3 4.6 mm*250 mm, 3 μm, column temperature: 35° C., MeOH (0.1% DEA) in CO₂, 0%-30.0%, flow rate: 2.0 mL/min. |
| 219 | 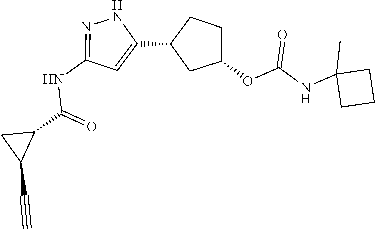 [SMILES: C#C[C@H]1C[C@@H]1C(=O)Nc1cc([C@@H]2CC[C@H](OC(=O)NC3(C)CCC3)C2)[nH]n1] | 371.1 | ¹H NMR (300 MHz, DMSO-d₆, ppm): δ 12.07 (brs, 1H), 10.72 (brs, 1H), 7.20-7.05 (m, 1H), 6.26 (s, 1H), 5.05-4.92 (m, 1H), 3.11-2.92 (m, 1H), 2.81 (d, J = 2.1 Hz, 1H), 2.50-2.40 (m, 1H), 2.29-2.09 (m, 3H), 2.02-1.51 (m, 10H), 1.30 (s, 3H), 1.20-1.03 (m, 2H). |
| 220 | 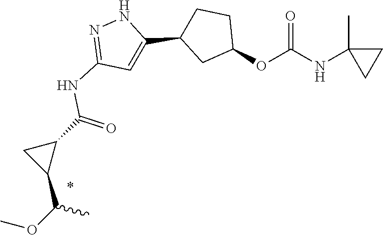 [SMILES: COC(C)[C@H]1C[C@@H]1C(=O)Nc1cc([C@H]2CC[C@@H](OC(=O)NC3(C)CC3)C2)[nH]n1] | 391.4 | ¹H NMR (400 MHz, METHANOL-d₄, ppm): δ 6.41-6.23 (m, 1H), 5.20-5.10 (m, 1H), 3.36 (s, 3H), 3.21-3.09 (m, 1H), 3.09-2.98 (m, 1H), 2.60-2.41 (m, 1H), 2.18-2.02 (m, 1H), 1.98-1.62 (m, 5H), 1.49-1.37 (m, 1H), 1.37-1.18 (m, 7H), 1.06-0.98 (m, 1H), 0.73-0.60 (m, 2H), 0.59-0.50 (m, 2H). Chiral purity: 100% ee, retention time: 1.866 min. Chiral SFC method: Waters UPCC (CA-060), column: DAICEL CHIRALPAK ®IA, 100*3.0 mm, 3 μm, column temperature: 35° C., MeOH (0.1% DEA) in CO₂, 40%, flow rate: 1.5 mL/min. |
| 221 | 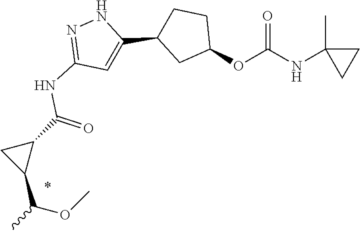 [SMILES: COC(C)[C@H]1C[C@@H]1C(=O)Nc1cc([C@H]2CC[C@@H](OC(=O)NC3(C)CC3)C2)[nH]n1] | 391.4 | ¹H NMR (400 MHz, METHANOL-d₄, ppm): δ 6.35-6.25 (m, 1H), 5.19-5.03 (m, 1H), 3.36 (s, 3H), 3.20-3.08 (m, 1H), 3.00-2.91 (m, 1H), 2.58-2.42 (m, 1H), 2.17-2.02 (m, 1H), 1.99-1.70 (m, 5H), 1.46-1.38 (m, 1H), 1.38-1.18 (m, 6H), 1.16-1.08 (m, 1H), 0.81-0.74 (m, 1H), 0.70-0.62 (m, 2H), 0.58-0.50 (m, 2H). Chiral purity: 98.08% de, retention time: 2.438 min. Chiral SFC method: Waters UPCC (CA-060), column: DAICEL CHIRALPAK ®IA, 100*3.0 mm, 3 μm, column temperature: 35° C., MeOH (0.1% |
| | | | DEA) in CO₂, 40%, flow rate: 1.5 |
| | | | mL/min. |
| 222 | 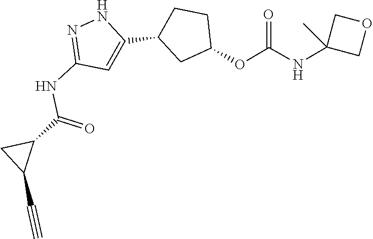 [SMILES: C#C[C@H]1C[C@@H]1C(=O)Nc1cc([C@@H]2CC[C@H](OC(=O)NC3(C)COC3)C2)[nH]n1] | 373.1 | ¹H NMR (300 MHz, METHANOL-d₄, ppm): δ 6.31 (s, 1H), 5.15-5.05 (m, 1H), 4.73 (s, 2H), 4.35 (d, J = 6.3 Hz, 2H), 3.21-3.12 (m, 1H), 2.58-2.42 (m, 1H), 2.23 (d, J = 2.1 Hz, 1H), 2.17-2.04 (m, 2H), 1.98-1.84 (m, 2H), 1.84-1.72 (m, 3H), 1.56 (s, 3H), 1.38-1.30 (m, 1H), 1.20-1.10 (m, 1H). |
| 223 | 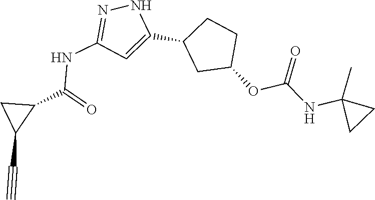 [SMILES: C#C[C@H]1C[C@@H]1C(=O)Nc1cc([C@@H]2CC[C@H](OC(=O)NC3(C)CC3)C2)[nH]n1] | 357.2 | ¹H NMR (300 MHz, DMSO-d₆, ppm): δ 12.06 (brs, 1H), 10.71 (brs, 1H), 7.33-7.07 (m, 1H), 6.25 (s, 1H), 5.03-4.90 (m, 1H), 3.11-2.94 (m, 1H), 2.81 (d, J = 2.1 Hz, 1H), 2.55-2.37 (m, 1H), 2.23-2.10 (m, 1H), 2.06-1.48 (m, 6H), 1.30-1.14 (m, 4H), 1.12-1.03 (m, 1H), 0.62-0.54 (m, 2H), 0.49-0.42 (m, 2H). |

### BIOLOGICAL ASSAYS AND DATA

### 1. Biochemical Assays

### 1.1 Materials

Kinases used-in this study are listed in the following table.

| Enzyme | Vendor | Cat# |
|---|---|---|
| CDK1/CyclinB1 | Thermo | PR4768C |
| CDK2/CyclinE1 | Carna | 04-165 |
| CDK4/CyclinD1 | Thermo | PV4400-10 |
| CDK5/p25 | Carna | 04-106 |
| CDK6/CyclinD3 | Carna | 04-107 |
| CDK7/CyclinH/MAT1 | Carna | 04-108 |
| CDK9/CyclinT1 | Carna | 04-110 |
| GSK3β | Carna | 04-141 |

Substrates and detection reagents used in this study are listed in the following table:

| Materials | Vendor | Cat# |
|---|---|---|
| Ulight-MBP peptide (0.5 nmoles) | PerkinElmer | TRF0109-D |
| EU-anti-P-MBP (10 μg) | PerkinElmer | TRF0201-D |
| ULight-4E-BP1 (Thr37/46) (5 nmoles) | PerkinElmer | TRF0128-M |
| Eu-anti-P-4E-BP1 (Thr37/46) (100 μg) | PerkinElmer | TRF0216-M |

### 1.2 Experimental Methods and Procedures

The purpose of these assays is to evaluate the inhibition of small molecule inhibitors by using TR-FRET assay.

Compound Preparation. Dissslove the compounds into DMSO solution to make 10 mM DMSO stock. Serially dilute the stock solution from highest concentration down to lowest in DMSO to make the compound stock plate (100× stock plates). To make 5× compound intermediate plate, add 38 μL of assay buffer (50 mM HEPES, pH 7.5, 0.05 mg/mL BSA, 10 mM MgCl₂, 1 mM EGTA, 0.01% Tween-20, 2 mM DTT) into each well of the V-bottom plate; then transfer 2 μL of the stock compound solution of each concentration from the compound stock plate (100× stock), and mix well.

Enzyme reaction. Transfer 2 μL compound from 5× intermediate plate into assay plate, then add 4 μL/well 2.5× Enzyme mix to assay plate, incubation for 10 min at room temperature. For positive control wells, add 4 μL/well assay buffer instead of Enzyme mix. Then add 4 μL/well 2.5× Substrate mix to assay plate, and incubation at 25° C. for 30 min at room temperature. After reaction, 10 μL 2× Detection mix (final 15 mM EDTA, 1× LANCE Detection Buffer, 0.5 nM Detection antibody) was added and incubated for 1 h prior to detection with excitation and emission at 320 and 665 nm (Tecan Spark® TR-FRET mode Tecan). Detail information of enzyme, substrate, detection antibody is listed in the following table.

| | Final | | | |
|---|---|---|---|---|
| | enzyme | ATP | Peptide | Detection |
| | conc. | conc. | substrate | antibody |
| Enzyme | (nM) | (μM) | (50 nM) | (0.5 nM) |
| CDK1/CyclinB | 7.5 | 17 | ULight-4E-BP1 | Eu-anti-P-4E-BP1 |
| | | | (Thr37/46) | (Thr37/46) |
| CDK2/CyclinE1 | 0.5 | 50 | ULight-4E-BP1 | Eu-anti-P-4E-BP1 |
| | | | (Thr37/46) | (Thr37/46) |
| GSK3β | 1.0 | 8.0 | ULight-4E-BP1 | Eu-anti-P-4E-BP1 |
| | | | (Thr37/46) | (Thr37/46) |
| CDK4/CyclinD1 | 0.50 | 225 | ULight-4E-BP1 | Eu-anti-P-4E-BP1 |
| | | | (Thr37/46) | (Thr37/46) |
| CDK5/p25 | 0.20 | 5 | ULight-4E-BP1 | Eu-anti-P-4E-BP1 |
| | | | (Thr37/46) | (Thr37/46) |
| CDK6/CyclinD3 | 0.50 | 140 | ULight-4E-BP1 | Eu-anti-P-4E-BP1 |
| | | | (Thr37/46) | (Thr37/46) |
| CDK7/CyclinH/ | 2.00 | 25 | ULIGHT-MBP | EU-anti-P-MBP |
| MAT1 | | | PEPTIDE | |
| CDK9/CyclinT1 | 1.0 | 3.5 | ULight-4E-BP1 | Eu-anti-P-4E-BP1 |
| | | | (Thr37/46) | (Thr37/46) |

### 1.3 Data Analysis

Inhibition rate (IR) of the tested compounds was determined by the following formula: IR (%)=(average High control−compound well)/(average High control−average Low control)*100%. Fit the compound IC₅₀ from non-linear regression equation: Y=Bottom+(Top−Bottom)/(1+10\hat{ }((Log IC₅₀−X)*HillSlope)). X: Log of cpd concentration; Y: Inhibition rate (TR); Top and Bottom: Plateaus in same units as Y, log IC₅₀: same log units as X; HillSlope: Slope factor or Hill slope.

### 2. Biological Activity Data

Biological activity data for CDK2 kinase assay are provided in Table 3:

TABLE 3

| | CDK2 IC₅₀ |
|---|---|
| Example# | (nM) |
| 1 | 7.1 |
| 2 | 17.8 |
| 3 | 16.1 |
| 4 | 2.5 |
| 5 | 0.8 |
| 6 | 0.8 |
| 7 | 2.4 |
| 8 | 11.3 |
| 9 | 1000 |
| 10 | 92.4 |
| 11 | 40.7 |
| 12 | 10.1 |
| 13 | 5.8 |
| 14 | 1.7 |
| 15 | 3.5 |
| 16 | 18.6 |
| 17 | 4.8 |
| 18 | 14.7 |
| 19 | 1000 |
| 20 | 1000 |
| 21 | 81.8 |
| 22 | 389 |
| 23 | 0.8 |
| 24 | 1.8 |
| 25 | 1.2 |
| 26 | 3.7 |
| 27 | 1.2 |
| 28 | 110 |
| 29 | 68.8 |
| 30 | 22.4 |
| 31 | 8.1 |
| 32 | 2.9 |
| 33 | 1.9 |
| 34 | 0.7 |
| 35 | 0.8 |
| 36 | 6.5 |
| 37 | 2.9 |
| 38 | 22.4 |
| 39 | 4.0 |
| 40 | 47.7 |
| 41 | 0.9 |
| 42 | 4.4 |
| 43 | 12.1 |
| 44 | 3.2 |
| 45 | 2.7 |
| 46 | 1.5 |
| 47 | 64.0 |
| 48 | 10.7 |
| 49 | 2.2 |
| 50 | 0.9 |
| 51 | 1.7 |
| 52 | 1.6 |
| 53 | 2.1 |
| 54 | 4.5 |
| 55 | 18.7 |
| 56 | 4.0 |
| 57 | 5.8 |
| 58 | 4.1 |
| 59 | 4.9 |
| 60 | 37.4 |
| 61 | 13.1 |
| 62 | 2.3 |
| 63 | 9.7 |
| 64 | 16.6 |
| 65 | 97.3 |
| 66 | 14.4 |
| 67 | 61.2 |
| 68 | 8.0 |
| 69 | 18.8 |
| 70 | 1.2 |
| 71 | 8.7 |
| 72 | 210 |
| 73 | 4.1 |
| 74 | 2.2 |
| 75 | 11.5 |
| 76 | 22.7 |
| 77 | 56.0 |
| 78 | 18.7 |
| 79 | 7.6 |
| 80 | 15.4 |
| 81 | 0.9 |
| 82 | 114 |
| 83 | 1.1 |
| 84 | 1.6 |
| 85 | 2.0 |
| 86 | 2.7 |
| 87 | 3.6 |
| 88 | 24.0 |
| 89 | 25.3 |
| 90 | 5.7 |
| 91 | 19.1 |
| 92 | 2.0 |
| 93 | 5.2 |
| 94 | 1.3 |
| 95 | 3.7 |
| 96 | 0.8 |
| 97 | 1.1 |
| 98 | 18.8 |
| 99 | 0.8 |
| 100 | 1.5 |
| 101 | 1.5 |
| 102 | 2.5 |
| 103 | 10.8 |
| 104 | 17.5 |
| 105 | 1.1 |
| 106 | 1.8 |
| 107 | 3.0 |
| 108 | 5.0 |
| 109 | 0.9 |
| 110 | 3.2 |
| 111 | 4.1 |
| 112 | 13.4 |
| 113 | 141 |
| 114 | 1.6 |
| 115 | 54.5 |
| 116 | 4.5 |
| 117 | 3.8 |
| 118 | 1.8 |
| 119 | 0.4 |
| 120 | 116 |
| 121 | 3.2 |
| 122 | 1.9 |
| 123 | 265 |
| 124 | 1.2 |
| 125 | 1.3 |
| 126 | 2.5 |
| 127 | 3.6 |
| 128 | 14.6 |
| 129 | 11.8 |
| 130 | 33.8 |
| 131 | 93.0 |
| 132 | 7.2 |
| 133 | 50.1 |
| 134 | 2.2 |
| 135 | 13.5 |
| 136 | 1.2 |
| 137 | 5.3 |
| 138 | 1.0 |
| 139 | 0.7 |
| 140 | 0.7 |
| 141 | 0.5 |
| 142 | 1.6 |
| 143 | 1.2 |
| 144 | 4.3 |
| 145 | 0.9 |
| 146 | 44.3 |
| 147 | 4.8 |
| 148 | 5.5 |
| 149 | 13.4 |
| 150 | 529 |
| 151 | 8.8 |
| 152 | 85.5 |
| 153 | 2.1 |
| 154 | 2.6 |
| 155 | 4.5 |
| 156 | 6.0 |
| 157 | 2.9 |
| 158 | 1.1 |
| 159 | 1.6 |
| 160 | 5.3 |
| 161 | 1.0 |
| 162 | 109 |
| 163 | 0.7 |
| 164 | 1.5 |
| 165 | 4.7 |
| 166 | 0.9 |
| 167 | 1.4 |
| 168 | 0.6 |
| 169 | 1.9 |
| 170 | 0.8 |
| 171 | 1.3 |
| 172 | 1.2 |
| 173 | 20.9 |
| 174 | 61.1 |
| 175 | 174 |
| 176 | 33.0 |
| 177 | 1.0 |
| 178 | 1.4 |
| 179 | 12.6 |
| 180 | 1.1 |
| 181 | 0.8 |
| 182 | 4.0 |
| 183 | 0.5 |
| 184 | 0.5 |
| 185 | 0.4 |
| 186 | 1.2 |
| 187 | 1.4 |
| 188 | 0.6 |
| 189 | 1.0 |
| 190 | 0.6 |
| 191 | 1.2 |
| 192 | 2.0 |
| 193 | 0.6 |
| 194 | 0.7 |
| 195 | 1.5 |
| 196 | 0.4 |
| 197 | 15.7 |
| 198 | 6.8 |
| 199 | 2.3 |
| 200 | 0.9 |
| 201 | 4.6 |
| 202 | 3.9 |
| 203 | 0.9 |
| 204 | 1.0 |
| 205 | 24.0 |
| 206 | 0.8 |
| 207 | 40.1 |
| 208 | 0.8 |
| 209 | 29.0 |
| 210 | 1.0 |
| 211 | 1.4 |
| 212 | 2.1 |
| 213 | 0.9 |
| 214 | 2.1 |
| 215 | 147 |
| 216 | 1.8 |
| 217 | 29.7 |
| 218 | 0.6 |
| 219 | 0.5 |
| 220 | 1.5 |
| 221 | 0.8 |
| 222 | 1.1 |
| 223 | 0.5 |

Biological activity data for CDK1 kinase assay are provided in Table 4.

TABLE 4

| | CDK1 IC₅₀ |
|---|---|
| Example# | (nM) |
| 14 | 96.7 |
| 15 | 142 |
| 24 | 102 |
| 25 | 54.9 |
| 33 | 118 |
| 44 | 255 |
| 49 | 132 |
| 52 | 111 |
| 53 | 134 |
| 97 | 72.6 |
| 100 | 74.8 |
| 102 | 133 |
| 122 | 89.4 |
| 126 | 142 |
| 136 | 64.6 |
| 143 | 60.9 |
| 180 | 69.4 |
| 181 | 34.4 |
| 189 | 58.6 |
| 191 | 101 |

Biological activity data for CDK9 kinase assay are provided in Table 5.

TABLE 5

| Example# | CDK9 IC₅₀ (nM) |
|---|---|
| 14 | 115 |
| 15 | 963 |
| 143 | 1000 |
| 180 | 210 |
| 191 | 131 |

Biological activity data for CDK4 and CDK6 kinase assays are provided in Table 6.

TABLE 6

| | CDK4 IC₅₀ | CDK6 IC₅₀ |
|---|---|---|
| Example# | (nM) | (nM) |
| 14 | 576 | 748 |
| 25 | 510 | 899 |
| 33 | 652 | 574 |
| 41 | 306 | 254 |
| 52 | 1000 | 1000 |

Biological activity data for GSK3β kinase assay are provided in Table 7.

TABLE 7

| | GSK3β IC₅₀ |
|---|---|
| Example# | (nM) |
| 41 | 572 |
| 49 | 395 |
| 52 | 1000 |
| 97 | 381 |
| 100 | 706 |

### 2. Cell Proliferation Assay

### 2.1 Materials

Medium and reagents used in this study are listed in the following table.

| Culture medium or reagent | Vendor | Cat# |
|---|---|---|
| ViewPlate-96 TC | PE | 6005181 |
| Master plate | Greiner bio-one | 651201 |
| T25 flask | Thermo Fisher | 156367 |
| FBS | Excell Bio | FND500 |
| 0.25% Trypsin | GIBCO | 25200-072 |
| RPMI1640 | GIBCO | 11875-093 |
| PBS | GIBCO | C14190500BT |
| Pen/Strep | BI | 03-031-1B |
| Insulin | BI | 41-975-100 |
| CellTiter-Glo ® Luminescent | Promega | G7573 |
| Cell Viability Assay | | |

### 2.2 Experimental Methods and Procedures

The OVCAR3 cancer cells were cultured at temperature 37° C., 5% CO₂ incubator. The cells were routinely subcultured. The cells growing in an exponential growth phase were harvested and counted for plating. Plate 100 μL of cell suspension into the assay plates. Incubate the assay plates at 37° C., 5% CO₂, 95% air and 100% relative humidity overnight. The compounds to be tested were dissolved in dimethyl sulfoxide and prepared with a stock concentration of 10 mM. Serially dilute the stock solution from highest concentration down to lowest in DMSO to make the compound stock plate (200× stock plates). Add 0.5 μL of the 200× compound of each well into the cells in assay plates. The final DMSO concentration was 0.5%. Return the assay plate into incubator and incubate for 6 days. The procedures were performed according to the Promega CellTiter-Glo Luminescent Cell Viability Assay Kit to detect cell viability.

### 2.3 Data Analysis

Inhibition rate (IR) of the tested compounds was determined by the following formula: IR (%)=(average High control−compound well)/(average High control−average Low control)*100%. Fit the compound IC₅₀ from non-linear regression equation: Y=Bottom+(Top−Bottom)/(1+10\hat{ }((Log IC₅₀−X)*HillSlope)). X: Log of cpd concentration; Y: Inhibition rate (IR); Top and Bottom: Plateaus in same units as Y; log IC₅₀: same log units as X; HillSlope: Slope factor or Hill slope.

Anti-proliferation data for OVCAR3 proliferation assay are provided in Table 8.

TABLE 8

| | OVCAR3 CTG |
|---|---|
| Example# | IC₅₀ (nM) |
| 41 | 193 |
| 84 | 74 |
| 109 | 237 |

The above description is only a specific embodiment of the present invention, but the scope of protection of the present invention is not limited thereto, and any person skilled in the art who is familiar with the technical field of the present invention can readily think of variations or substitutions within the technical scope of the present invention as disclosed herein shall be covered within the scope of protection of the present invention. Therefore, the scope of protection of the present invention shall be stated to be subject to the scope of protection of the claims.

1. A compound represented by formula 1a or formula 1b, or an optical isomer, prodrug or pharmaceutically acceptable salt thereof: 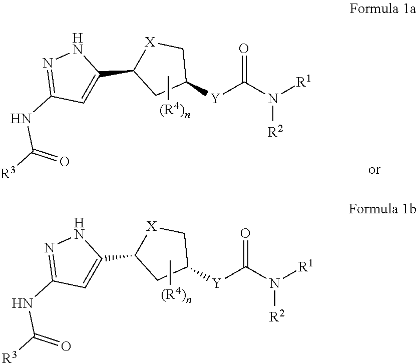
wherein,
X is selected from C, O or S, and X bonds with 1 or 2 hydrogen atoms or without hydrogen atoms attached according to the bonding valence;
Y is selected from C, O or N, preferably O or N and Y bonds with 1 or 2 hydrogen atoms or without hydrogen atoms attached according to the bonding valence; or Y together with the 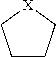
to form an optionally substituted 6 to 8 membered aliphatic fused ring, or an optionally substituted 6 to 8 membered aliphatic hetero fused ring containing 1 to 3 heteroatoms selected from N, O, and S;
R¹ and R² are independently hydrogen, optionally substituted C1 to C8 alkyl, optionally substituted C2 to C8 alkoxy, optionally substituted C3 to C15 cycloalkyl, optionally substituted 4 to 15 membered heterocycloalkyl containing 1 to 3 heteroatoms selected from N, O, and S, optionally substituted 4 to 15 membered aliphatic fused ring, optionally substituted 5 to 15 membered aliphatic spiro ring, optionally substituted 5 to 15 membered aliphatic bridged ring, optionally substituted 5 to 15 membered aliphatic hetero fused ring containing 1 to 3 heteroatoms selected from N, O, and S, optionally substituted 6 to 15 membered aliphatic hetero spiro ring containing 1 to 3 heteroatoms selected from N, O, and S, or optionally substituted 5 to 15 membered aliphatic hetero bridged ring containing 1 to 3 heteroatoms selected from N, O, and S heteroatom; or R¹ and R² are joined to form an optionally substituted 4 to 15 membered heterocycloalkyl containing 1 to 3 heteroatoms selected from N, O, and S, an optionally substituted 5 to 15 membered aliphatic hetero fused ring containing 1 to 3 heteroatoms selected from N, O, and S in addition to the N atom to which R¹ and R² are bonded, an optionally substituted 6 to 15 membered aliphatic hetero spiro ring containing 1 to 3 heteroatoms selected from N, O, and S in addition to the N atom to which R¹ and R² are bonded, or an optionally substituted 5 to 15 membered aliphatic hetero bridged ring containing 1 to 3 heteroatoms selected from N, O, and S in addition to the N atom to which R¹ and R² are bonded; or one of R¹ and R², together with the N atom to which R¹ and R² are bonded, the carbonyl group and Y, forms an optionally substituted 5 to 10 membered heterocycloalkyl containing 1 to 3 heteroatoms selected from N, O, and S;
wherein the “substituted” in the above definition of R¹ and R² means that the group comprises 1 to 3 substituents selected from C1 to C6 alkyl, C1 to C6 haloalkyl, C1 to C6 alkoxy, C1 to C6 haloalkoxy, C2 to C6 alkenyl, C2 to C6 haloalkenyl, C2 to C6 alkynyl, C2 to C6 haloalkynyl, C3 to C6 cycloalkyl, C3 to C6 halocycloalkyl, C4 to C6 heterocycloalkyl, C4 to C6 haloheterocycloalkyl, hydroxyl, amino, sulfone group, cyano, and halogen atom;
R³ is selected from hydrogen, optionally substituted C1 to C15 alkyl, optionally substituted C2 to C15 alkenyl, optionally substituted C2 to C15 alkynyl, optionally substituted C2 to C15 alkoxy, optionally substituted C3 to C15 cycloalkyl, optionally substituted 4 to 15 membered heterocycloalkyl containing 1 to 3 heteroatoms selected from N, O, and S, optionally substituted C6 to C14 aryl, optionally substituted 5 to 15 membered heteroaryl containing 1 to 3 heteroatoms selected from N, O, and S, optionally substituted 4 to 17 membered aliphatic fused ring, optionally substituted 5 to 17 membered aliphatic bridged ring, optionally substituted 5 to 17 membered aliphatic spiro ring, optionally substituted 4 to 17 membered aliphatic hetero fused ring containing 1 to 3 heteroatoms selected from N, O, and S, optionally substituted 5 to 17 membered aliphatic hetero bridged ring containing 1 to 3 heteroatoms selected from N, O and S heteroatoms, optionally substituted 5 to 17 membered aromatic fused ring, optionally substituted 5 to 17 membered aromatic hetero fused ring containing 1 to 3 heteroatoms selected from N, O, and S, and optionally substituted 4 to 17 membered aliphatic hetero spiro ring containing 1 to 3 heteroatoms selected from N, O, and S, C8-C14 cycloalkyl fused heteroaryl and C8-C14 heterocycloalkyl fused heteroaryl, wherein the “substituted” means that the group comprises 1 to 3 substituents selected from the following groups: optionally substituted C1 to C6 alkyl, optionally substituted C1 to C6 alkoxy, hydroxy-substituted C1 to C6 alkyl group, amino-substituted C1 to C6 alkyl group, C1 to C6 alkyl group substituted with 1, 2, or 3 halogen atoms, C1 to C6 alkoxy substituted with 1, 2, or 3 halogen atoms, optionally substituted C2 to C7 alkoxyalkyl, C2 to C7 alkoxyalkyl substituted with 1, 2, or 3 halogen atoms, optionally substituted C2 to C6 alkenyl, optionally substituted C2 to C6 alkynyl, optionally substituted C3 to C6 cycloalkyl, optionally substituted 4 to 6 membered heterocycloalkyl containing 1 to 3 heteroatoms selected from N, O, and S, optionally substituted C6 to C10 aryl, optionally substituted 5 to 10 membered heteroaryl containing 1 to 3 heteroatoms selected from N, O, and S, optionally substituted 6 to 10 membered fused heterocyclyl containing 1 to 3 heteroatoms selected from N, O, and S, optionally substituted 9 to 16 membered cycloalkyl fused aryl, optionally substituted 9 to 16 membered heterocycloalkyl fused aryl containing 1 to 3 heteroatoms selected from N, O and S, optionally substituted 8 to 15 membered cycloalkyl fused heteroaryl containing 1 to 5 heteroatoms selected from N, O and S, and optionally substituted 8 to 15 membered heterocycloalkyl fused heteroaryl containing 1 to 5 heteroatoms selected from N, O and S, optionally substituted C3 to C6 cycloalkylsulfonyl, optionally substituted C1 to C6 alkylsulfonyl, C1 to C6 haloalkyl sulfonyl, optionally substituted C1 to C6 alkylacyl, C1 to C6 haloalkylacyl, optionally substituted C6 to C14 arylacyl, optionally substituted 5 to 15 membered heteroarylacyl containing 1 to 3 heteroatoms selected from N, O, and S, cyano, and halo;
R⁴ is selected from hydrogen, hydroxyl, cyano, halogen atom, C1 to C6 alkyl, and C1 to C6 alkoxy; or
R⁴ together with the 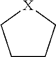
to form an optionally substituted 6 to 8 membered aliphatic fused ring, or an optionally substituted 6 to 8 membered aliphatic hetero fused ring containing 1 to 3 heteroatoms selected from N, O, and S, wherein the “substituted” means that the group comprises 1 to 3 substituents selected from C1 to C6 alkyl, C1 to C6 alkoxy, C2 to C6 alkenyl, C2 to C6 alkynyl, C3 to C6 cycloalkyl, cyano, hydroxyl, halogen atom;
the subscript n is an integer from 0 to 4.

2. The compound, or an optical isomer, prodrug or pharmaceutically acceptable salt thereof according to claim 1, characterized in that the subscript n is 0, 1, 2, 3, or 4.

3. The compound, or an optical isomer, prodrug or pharmaceutically acceptable salt thereof according to claim 1, characterized in that the subscript n is 0, 1, 2, or 3.

4. The compound, or an optical isomer, prodrug or pharmaceutically acceptable salt thereof according to claim 1, characterized in that the subscript n is 0, 1, or 2.

5. The compound, or an optical isomer, prodrug or pharmaceutically acceptable salt thereof according to claim 1, characterized in that the subscript n is 0 or 1.

6. The compound, or an optical isomer, prodrug or pharmaceutically acceptable salt thereof according to claim 1, characterized in that
R¹ and R² are the same or different from each other and are each independently selected from hydrogen, optionally substituted C1 to C6 alkyl, optionally substituted C2 to C6 alkoxy, optionally substituted C3 to C10 cycloalkyl, optionally substituted 4 to 10 membered heterocycloalkyl containing 1 to 3 heteroatoms selected from N, O, and S, optionally substituted 4 to 13 membered aliphatic fused ring, optionally substituted 5 to 13 membered aliphatic spiro ring, optionally substituted 5 to 13 membered aliphatic bridged ring, optionally substituted 5 to 13 membered aliphatic hetero fused ring containing 1 to 3 heteroatoms selected from N, O, and S, optionally substituted 6 to 13 membered aliphatic hetero spiro ring containing 1 to 3 heteroatoms selected from N, O, and S, and optionally substituted 5 to 13 membered aliphatic hetero bridged ring containing 1 to 3 heteroatoms selected from N, O, and S; or
R¹ and R² together with the N atom to which they are bonded form an optionally substituted 4 to 10 membered heterocycloalkyl containing 1 to 3 heteroatoms selected from N, O, and S, an optionally substituted 5 to 10 membered aliphatic hetero fused ring containing 1 to 3 heteroatoms selected from N, O, and S in addition to the N atom to which R¹ and R² are bonded, an optionally substituted 6 to 13 membered aliphatic hetero spiro ring containing 1 to 3 heteroatoms selected from N, O, and S in addition to the N atom to which R¹ and R² are bonded, or a optionally substituted 5 to 13 membered aliphatic hetero bridged ring containing 1 to 3 heteroatoms selected from N, O, and S in addition to the N atom to which R¹ and R² are bonded; or
one of R¹ and R², together with the N atom to which they are bonded, the carbonyl group and Y, forms an optionally substituted 5 to 8 membered heterocycloalkyl containing 1 to 3 heteroatoms selected from N, O, and S.

7. The compound, or an optical isomer, prodrug or pharmaceutically acceptable salt thereof according to claim 1, characterized in that
R¹ and R² are the same or different from each other and are each independently selected from hydrogen, optionally substituted C1 to C4 alkyl, optionally substituted C2 to C4 alkoxy, optionally substituted C3 to C8 cycloalkyl, optionally substituted 4 to 8 membered heterocycloalkyl containing 1 to 3 heteroatoms selected from N, O, and S, optionally substituted 4 to 10 membered aliphatic fused ring, optionally substituted 5 to 10 membered aliphatic spiro ring, optionally substituted 5 to 10 membered aliphatic bridged ring, optionally substituted 5 to 10 membered aliphatic hetero fused ring containing 1 to 3 heteroatoms selected from N, O, and S, optionally substituted 6 to 10 membered aliphatic hetero spiro ring containing 1 to 3 heteroatoms selected from N, O, and S, and optionally substituted 5 to 10 membered aliphatic hetero bridged ring containing 1 to 3 heteroatoms selected from N, O, and S; or
R¹ and R² together with the N atom to which they are bonded form an optionally substituted 4 to 8 membered heterocycloalkyl containing 1 to 3 heteroatoms selected from N, O, and S, an optionally substituted 4 to 10 membered aliphatic hetero fused ring containing 1 to 3 heteroatoms selected from N, O, and S in addition to the N atom to which R¹ and R² are bonded, an optionally substituted 6 to 10 membered aliphatic hetero spiro ring containing 1 to 3 heteroatoms selected from N, O, and S in addition to the N atom to which R¹ and R² are bonded, or an optionally substituted 5 to 10 membered aliphatic hetero bridged ring containing 1 to 3 heteroatoms selected from N, O, and S in addition to the N atom to which R¹ and R² are bonded; or
one of R¹ and R², together with the N atom to which they are bonded, the carbonyl group and Y forms an optionally substituted 5 to 6 membered heterocycloalkyl containing 1 to 3 heteroatoms selected from N, O, and S.

8. The compound, or an optical isomer, prodrug or pharmaceutically acceptable salt thereof according to claim 1, characterized in that
R1 and R2 are independently selected from hydrogen, methyl, ethyl, n-propyl, isopropyl, 1,1-difluoroisopropyl, n-butyl, isobutyl, tert-butyl, ethoxy, n-propoxy, isopropoxy, n-butoxy, isobutoxy, tert-butoxy, cyclopropyl, cyclobutyl, cyclopentyl, cyclohexyl, methylcyclopropyl, ethylcyclopropyl, n-propylcyclopropyl, isopropylcyclopropyl, n-butylcyclopropyl, isobutylcyclopropyl, methylcyclobutyl, ethylcyclobutyl, n-propylcyclobutyl, isopropylcyclobutyl, n-butylcyclobutyl, isobutylcyclobutyl, methylcyclopentyl, ethylcyclopentyl, n-propylcyclopentyl, isopropylcyclopentyl n-butylcyclopentyl, isobutylcyclopentyl, oxetanyl, tetrahydrofuranyl, tetrahydropyranyl, azetidinyl, pyrrolidinyl, piperidinyl, methyloxetanyl, ethyloxetanyl, n-propyloxetanyl, isopropyloxetanyl, n-butyloxetanyl, isobutyloxetanyl, methyl tetrahydrofuranyl, ethyl tetrahydrofuranyl, n-propyl tetrahydrofuranyl, isopropyl tetrahydrofuranyl, n-butyl tetrahydrofuranyl, isobutyl tetrahydrofuranyl, methylazetidinyl, ethylazetidinyl, n-propylazetidinyl, isopropylazetidinyl, n-butylazetidinyl, isobutylazetidinyl, methylpyrrolidinyl, ethylpyrrolidinyl, n-propylpyrrolidinyl, isopropylpyrrolidinyl, n-butylpyrrolidinyl, isobutylpyrrolidinyl, 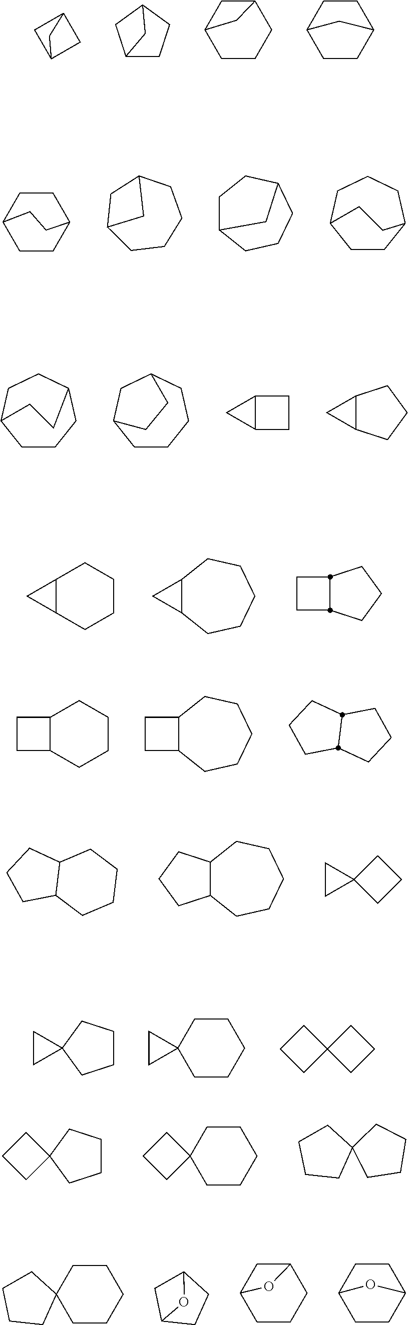 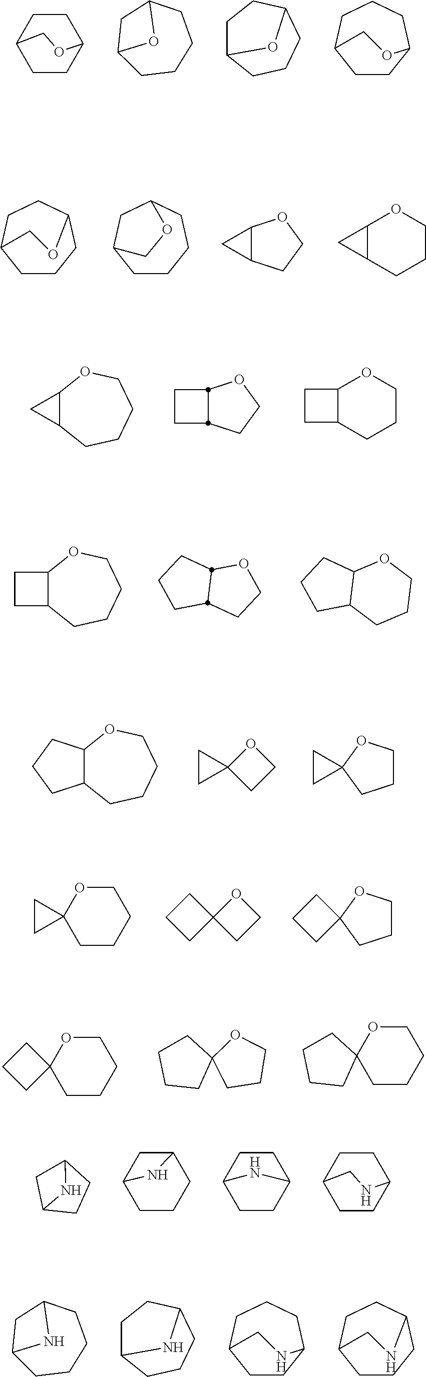 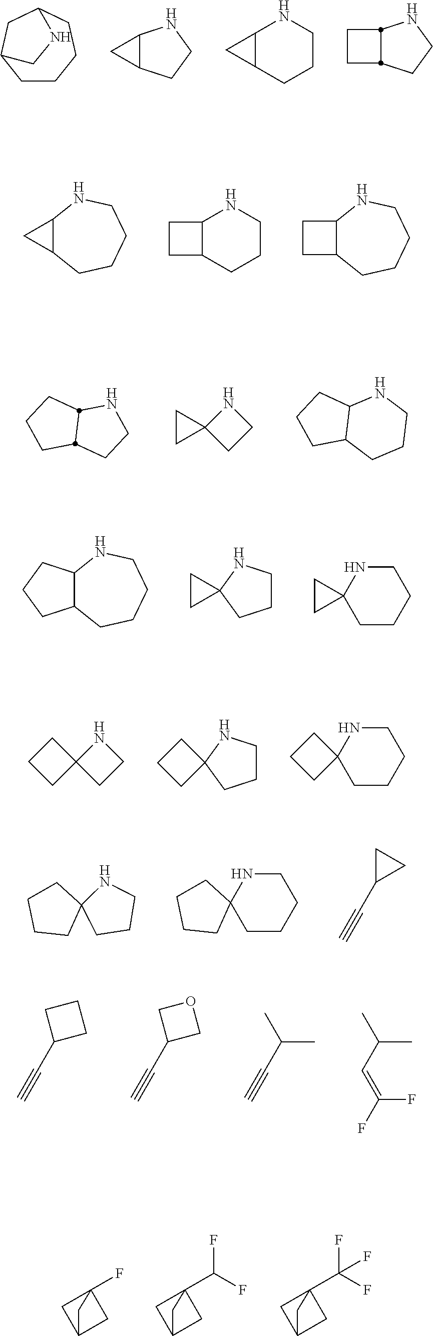 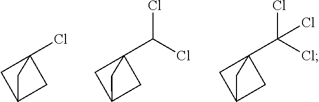
or
R¹ and R², together to form an optionally substituted 4 to 8 membered heterocycloalkyl containing 1 to 3 heteroatoms selected from N, O, and S, an optionally substituted 5 to 10 membered aliphatic hetero fused ring containing 1 to 4 heteroatoms selected from N, O, and S, an optionally substituted 6 to 10 membered aliphatic hetero spiro ring containing 1 to 4 heteroatoms selected from N, O, and S, or an optionally substituted 5 to 10 membered aliphatic hetero bridged ring containing 1 to 4 heteroatoms selected from N, O, and S; or
one of R¹ and R², together with the N atom to which they are bonded, the carbonyl and Y, forms an optionally substituted 5 to 6 membered heterocycloalkyl containing 1 to 3 heteroatoms selected from N, O, and S.

9. The compound, or an optical isomer, prodrug or pharmaceutically acceptable salt thereof according to claim 1, characterized in that
R⁴ is selected from hydrogen, C1 to C3 alkyl, and C1 to C3 alkoxy, or
R⁴ together with the 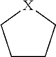
to which R⁴ is bonded forms

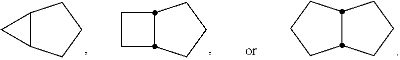

10. The compound, or an optical isomer, prodrug or pharmaceutically acceptable salt thereof according to claim 1, characterized in that, the compound is a compound represented by the following formula 1-1a or formula 1-1b: 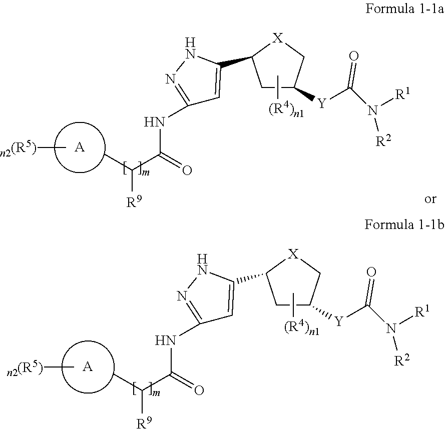
wherein,
X, Y, R¹, R², and R⁴ are defined the same as those in Formula 1a or Formula 1b, and n1 is defined the same as n in Formula 1a or Formula 1b in claim 1; 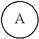
is selected from optionally substituted C3 to C15 cycloalkyl, optionally substituted 4 to 15 membered heterocycloalkyl containing 1 to 3 heteroatoms selected from N, O, and S, optionally substituted 6 to 14 membered aryl, optionally substituted 5 to 15 membered heteroaryl containing 1 to 3 heteroatoms selected from N, O, and S, optionally substituted 4 to 17 membered aliphatic fused ring, optionally substituted 5 to 17 membered aliphatic bridged ring, optionally substituted 5 to 17 membered aliphatic spiro ring, optionally substituted 4 to 17 membered aliphatic hetero fused ring containing 1 to 3 heteroatoms selected from N, O, and S, optionally substituted 5 to 17 membered aliphatic hetero bridged ring containing 1 to 3 heteroatoms selected from N, O, and S, optionally substituted 5 to 17 membered aliphatic hetero spiro ring containing 1 to 3 heteroatoms selected from N, O, and S, and optionally substituted 5 to 17 membered aromatic hetero fused ring containing 1 to 3 heteroatoms selected from N, O, and S, C8-C14 cycloalkyl fused heteroaryl and C8-C14 heterocycloalkyl fused heteroaryl;
R⁵ is selected from hydrogen, optionally substituted C1 to C6 alkyl, optionally substituted C1 to C6 alkylacyl, optionally substituted C1 to C6 alkoxy, optionally substituted C1 to C6 alkoxyacyl, C1 to C6 alkoxy substituted with C1 to C6 alkyl, optionally substituted C2 to C6 alkenyl, optionally substituted C2 to C6 alkynyl, sulfonyl, optionally substituted C1 to C6 alkylsulfonyl, optionally substituted C3 to C6 cycloalkylsulfonyl, C1 to C6 haloalkylsulfonyl, carbonyl, cyano, halogen atom, hydroxyl, C1 to C6 haloalkylacyl, optionally substituted C6 to C14 arylacyl, optionally substituted 5 to 15 membered heteroarylacyl containing 1 to 3 heteroatoms selected from N, O, and S, optionally substituted C3 to C8 cycloalkyl, saturated or unsaturated 4 to 8 membered heterocycloalkyl containing 1 to 3 heteroatoms selected from N, O, and S, optionally substituted 6 to 10 membered aryl, optionally substituted 5 to 10 membered heteroaryl containing 1 to 3 heteroatoms selected from N, O, and S, optionally substituted 8 to 15 membered aromatic fused ring, optionally substituted 8 to 15 membered aromatic hetero fused ring containing 1 to 3 heteroatoms selected from N, O, and S, optionally substituted 9 to 16 membered cycloalkyl fused aryl, optionally substituted 9 to 16 membered heterocycloalkyl fused aryl containing 1 to 3 heteroatoms selected from N, O and S, optionally substituted 8 to 15 membered cycloalkyl fused heteroaryl containing 1 to 5 heteroatoms selected from N, O and S, and optionally substituted 8 to 15 membered heterocycloalkyl fused heteroaryl containing 1 to 5 heteroatoms selected from N, O and S, wherein the “substituted” means that the group comprises 1 to 3 substituents selected from C1 to C6 alkyl, C1 to C6 alkoxy, C1 to C6 alkyl substituted with 1, 2, or 3 halogen atoms, C1 to C6 alkoxy substituted with 1, 2, or 3 halogen atoms, C2 to C6 alkenyl, C2 to C6 alkynyl, C3 to C6 cycloalkyl, cyano, nitro, halogen atom, hydroxyl, amino, amide, hydroxylamino, C3 to C6 cycloalkyl, 3 to 6 membered heterocycloalkyl containing 1 to 3 heteroatoms selected from N, O, and S, 6 to 8 membered aryl, and 3 to 6 membered heteroaryl containing 1 to 3 heteroatoms selected from N, O, and S;
n2 is an integer from 0 to 4;
R⁹ is selected from hydrogen, C1 to C4 alkyl, C1 to C4 alkoxy, C1 to C4 haloalkyl, C1 to C4 haloalkoxy;
m is an integer from 0 to 4.

11. The compound, or an optical isomer, prodrug or pharmaceutically acceptable salt thereof according to claim 10, characterized in that n2 is 0, 1, 2, 3 or 4.

12. The compound, or an optical isomer, prodrug or pharmaceutically acceptable salt thereof according to claim 10, characterized in that n2 is 0, 1, 2 or 3.

13. The compound, or an optical isomer, prodrug or pharmaceutically acceptable salt thereof according to claim 10, characterized in that n2 is 0, 1 or 2.

14. The compound, or an optical isomer, prodrug or pharmaceutically acceptable salt thereof according to claim 10, characterized in that m is 0, 1, 2, 3, or 4.

15. The compound, or an optical isomer, prodrug or pharmaceutically acceptable salt thereof according to claim 10, characterized in that m is 0, 1, 2 or 3.

16. The compound, or an optical isomer, prodrug or pharmaceutically acceptable salt thereof according to claim 10, characterized in that m is 0, 1, or 2.

17. The compound, or an optical isomer, prodrug or pharmaceutically acceptable salt thereof according to claim 10, characterized in that 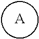
is selected from optionally substituted C3 to C10 cycloalkyl, optionally substituted 3 to 10 membered heterocycloalkyl containing 1 to 3 heteroatoms selected from N, O and S, optionally substituted 6 to 10 membered aryl, optionally substituted 5 to 10 membered heteroaryl containing 1 to 3 heteroatoms selected from N, O, and S, optionally substituted 4 to 13 membered aliphatic fused ring, optionally substituted 5 to 13 membered aliphatic bridged ring, optionally substituted 4 to 13 membered aliphatic hetero fused ring containing unsubstituted 1 to 3 heteroatoms selected from N, O, and S, optionally substituted 5 to 13 membered aliphatic hetero bridged ring containing 1 to 3 heteroatoms selected from N, O and S, optionally substituted 10 to 14 membered aromatic fused ring, and optionally substituted 8 to 12 membered aromatic hetero fused ring containing 1 to 3 heteroatoms selected from N, O, and S.

18. The compound, or an optical isomer, prodrug or pharmaceutically acceptable salt thereof according to claim 10, characterized in that 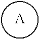
is selected from optionally substituted C3 to C8 cycloalkyl, optionally substituted 3 to 8 membered heterocycloalkyl containing 1 to 3 heteroatoms selected from N, O and S, optionally substituted 6 to 8 membered aryl, optionally substituted 5 to 8 membered heteroaryl containing 1 to 3 heteroatoms selected from N, O and S, optionally substituted 4 to 10 membered aliphatic fused ring, optionally substituted 4 to 10 membered aliphatic spiro ring, saturated or unsaturated substituted unsubstituted 5 to 10 membered aliphatic bridged ring, optionally substituted 4 to 10 membered aliphatic hetero fused ring containing 1 to 3 heteroatoms selected from N, O and S, optionally substituted 5 to 10 membered aliphatic hetero spiro ring containing 1 to 3 heteroatoms selected from N, O and S, optionally substituted 5 to 10 membered aliphatic hetero bridged ring containing 1 to 3 heteroatoms selected from N, O and S.

19. The compound, or an optical isomer, prodrug or pharmaceutically acceptable salt thereof according to claim 10, characterized in that 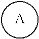
is selected from:

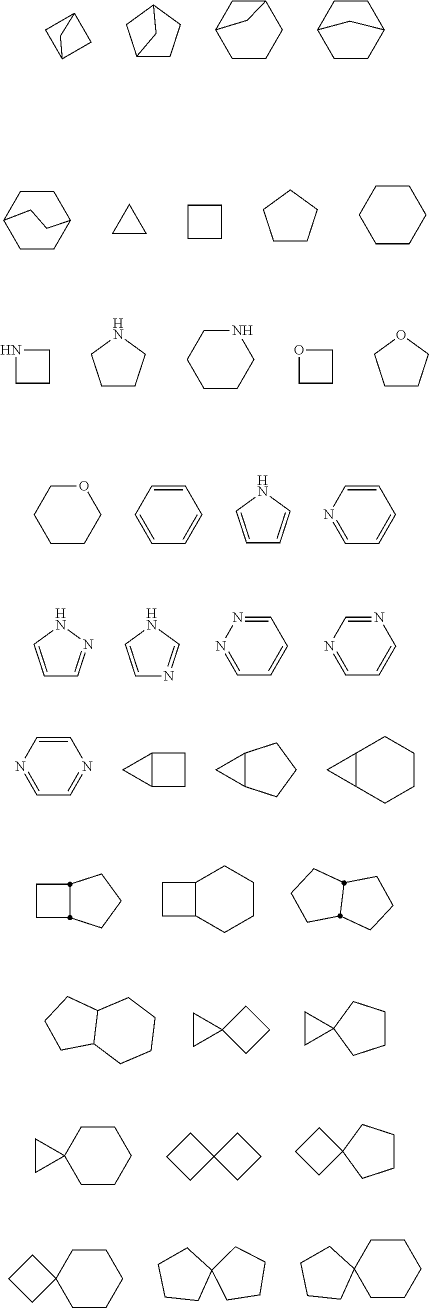

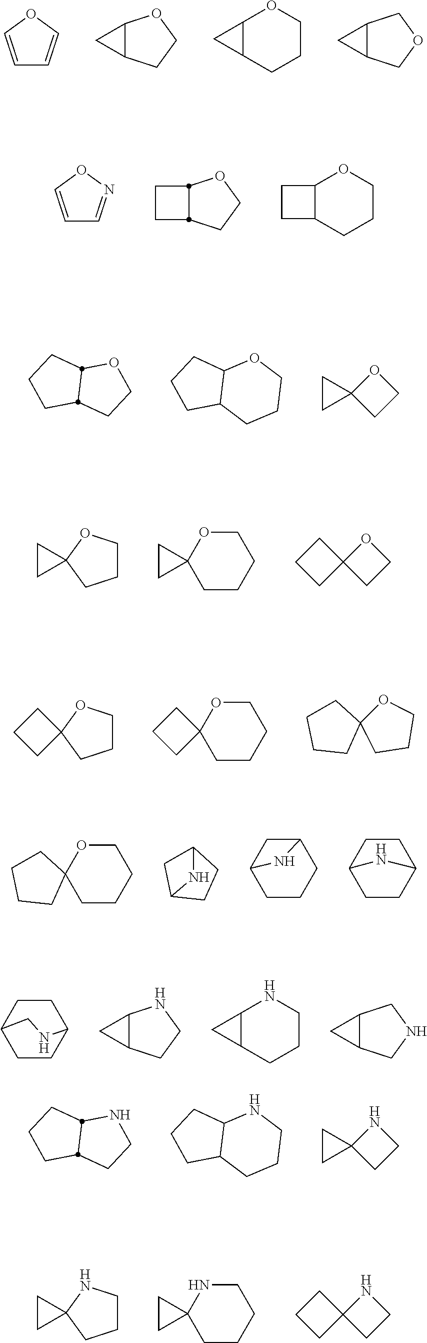

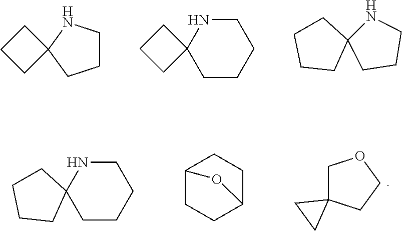

20. The compound, or an optical isomer, prodrug or pharmaceutically acceptable salt thereof according to claim 10, characterized in that,
R⁹ is selected from hydrogen, methyl, ethyl, n-propyl, isopropyl, n-butyl, isobutyl, tert-butyl, methoxy, ethoxy, n-propyloxy, isopropoxy, n-butoxy, isobutoxy, tert-butoxy, monofluoromethyl, difluoromethyl, trifluoromethyl, dichloromethyl, trichloromethyl, monofluoroethyl, difluoroethyl, trifluoroethyl, tetrafluoroethyl, pentafluoroethyl, dichloroethyl, trichloroethyl, tetrachloroethyl, pentachloroethyl, difluoropropyl, trifluoropropyl, tetrafluoropropyl, pentafluoropropyl, hexafluoropropyl, perfluoropropyl, monochloropropyl, dichloropropyl, trichloropropyl, tetrachloropropyl, pentachloropropyl, hexachloropropyl, perchloropropyl.

21. The compound, or an optical isomer, prodrug or pharmaceutically acceptable salt thereof according to claim 1, characterized in that, the compound is a compound represented by the following formula 1-1-1a, formula 1-1-1b, formula 1-1-2a, formula 1-1-2b formula 1-1-3a formula 1-1-3b formula 1-1-4a or formula 1-1-4b: 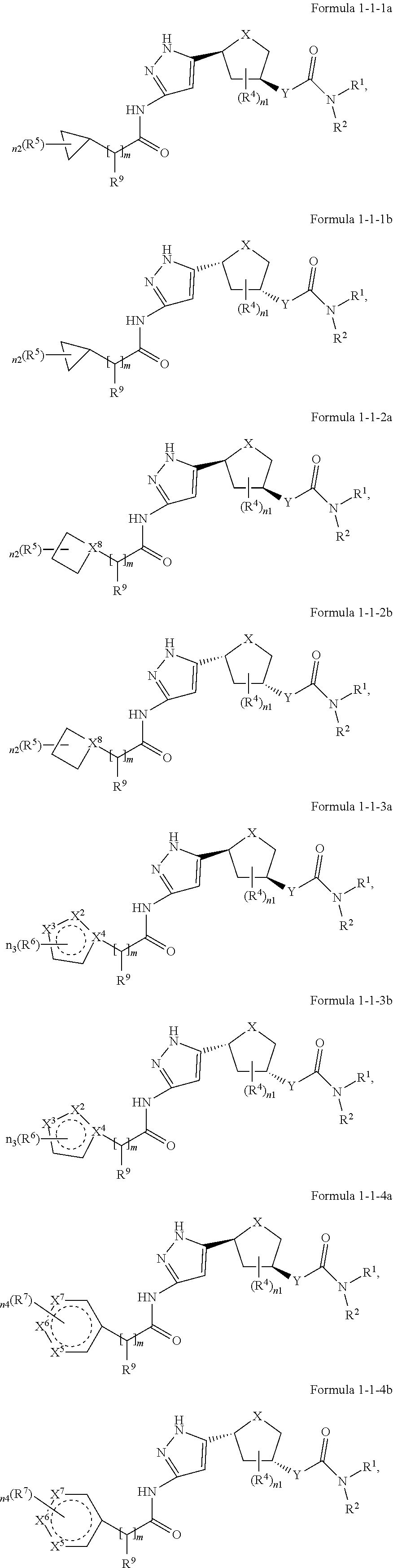
wherein,
X, Y, R¹, R², R⁴ are defined the same as those in Formula 1a or Formula 1b, n1 is defined the same as n in Formula 1a or Formula 1b;
R⁵, R⁹ and m are defined the same as those in formula 1-1a or formula 1-1b;
X² to X⁸ are each independently selected from C, O, S, and N, and X² to X⁸ each independently bond with 1 or 2 hydrogen atoms or without hydrogen atoms attached according to the bonding valence;
R⁶ is selected from hydrogen, C1 to C6 alkyl, C1 to C6 alkoxy, C2 to C8 alkoxyalkyl, C1 to C6 haloalkyl, C1 to C6 haloalkoxy, C1 to C6 alkylacyl, C3 to C6 cycloalkyl, C3 to C6 cycloalkylacyl, 4 to 6 membered heterocycloalkyl containing 1 to 3 heteroatoms selected from N, O and S, 4 to 6 membered heterocycloalkylacyl containing 1 to 3 heteroatoms selected from N, O, and S, sulfonyl, C3 to C6 cycloalkylsulfonyl, C1 to C6 alkylsulfonyl, C1 to C6 haloalkylsulfonyl, optionally substituted C6 to C14 arylacyl, optionally substituted 5 to 15 membered heteroarylacyl containing 1 to 3 heteroatoms selected from N, O, and S, carbonyl, cyano, and halogen atoms;
R⁷ is selected from hydrogen, C1 to C6 alkyl, C1 to C6 haloalkyl group, C1 to C6 alkoxy, C2 to C8 alkoxyalkyl, sulfonyl, C3 to C6 cycloalkylsulfonyl, C1 to C6 alkylsulfonyl, C1 to C6 haloalkylsulfonyl, C1 to C6 alkylacyl, C1 to C6 haloalkylacyl, optionally substituted C6 to C15 arylacyl, optionally substituted 5 to 15 membered heteroaryl acyl containing 1 to 3 heteroatoms selected from N, O, and S, carbonyl, cyano, halogen atoms, and C1 to C6 alkoxy substituted by 4 to 6 membered heterocycloalkyl containing 1 to 3 heteroatoms selected from N, O, and S;
the subscripts n3 and n4 are each independently an integer from 0 to 4.

22. The compound, or an optical isomer, prodrug or pharmaceutically acceptable salt thereof according to claim 21, characterized in that, X² to X⁸ are each independently selected from C, O, and N, and X² to X⁸ each independently bond with 1 or 2 hydrogen atoms or without hydrogen atoms attached according to the bonding valence.

23. The compound, or an optical isomer, prodrug or pharmaceutically acceptable salt thereof according to claim 21, characterized in that, n3 and n4 are each independently 0, 1, 2, 3, or 4, respectively.

24. The compound, or an optical isomer, prodrug or pharmaceutically acceptable salt thereof according to claim 21, characterized in that, n3 and n4 are each independently 0, 1, 2, or 3, respectively.

25. The compound, or an optical isomer, prodrug or pharmaceutically acceptable salt thereof according to claim 21, characterized in that, n3 and n4 are each independently 0, 1, or 2, respectively.

26. The compound, or an optical isomer, prodrug or pharmaceutically acceptable salt thereof according to claim 1, characterized in that, the compound is selected from the following specific compounds:

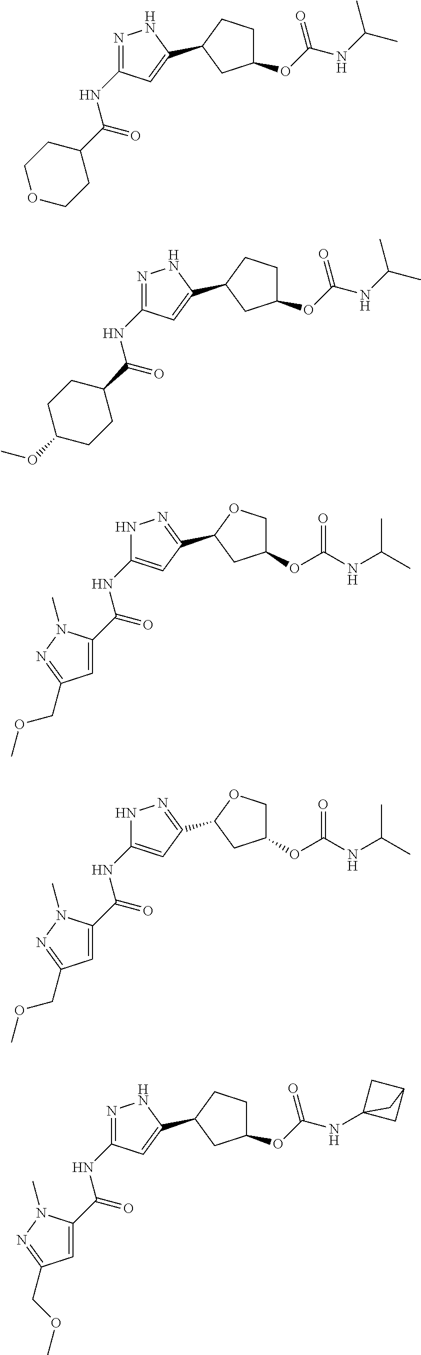

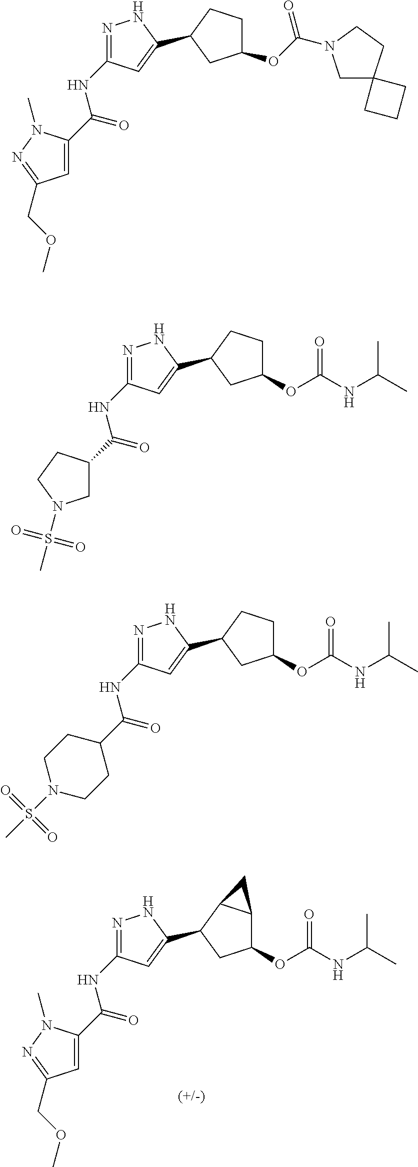

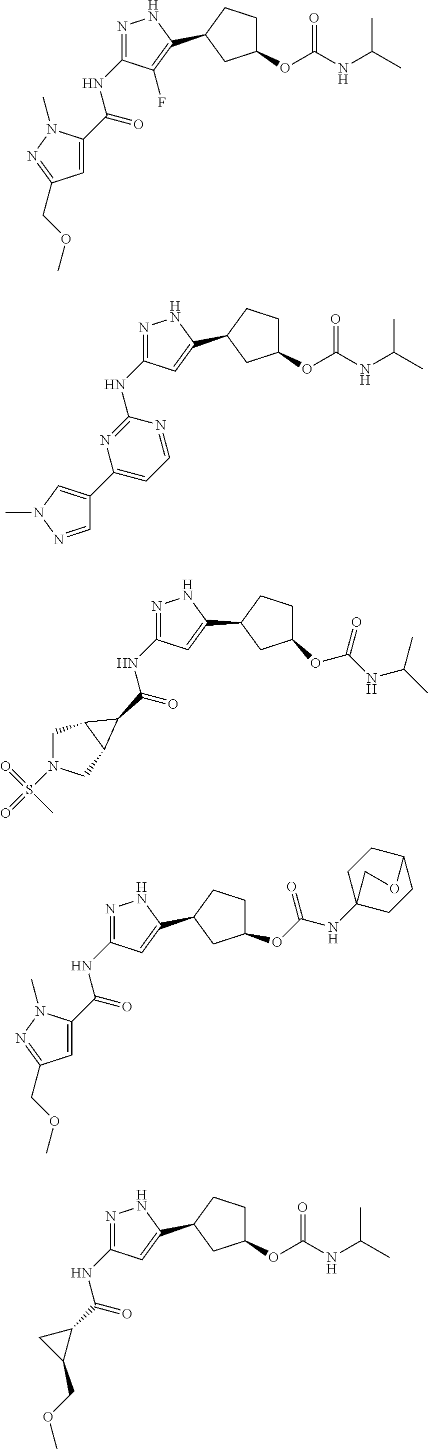

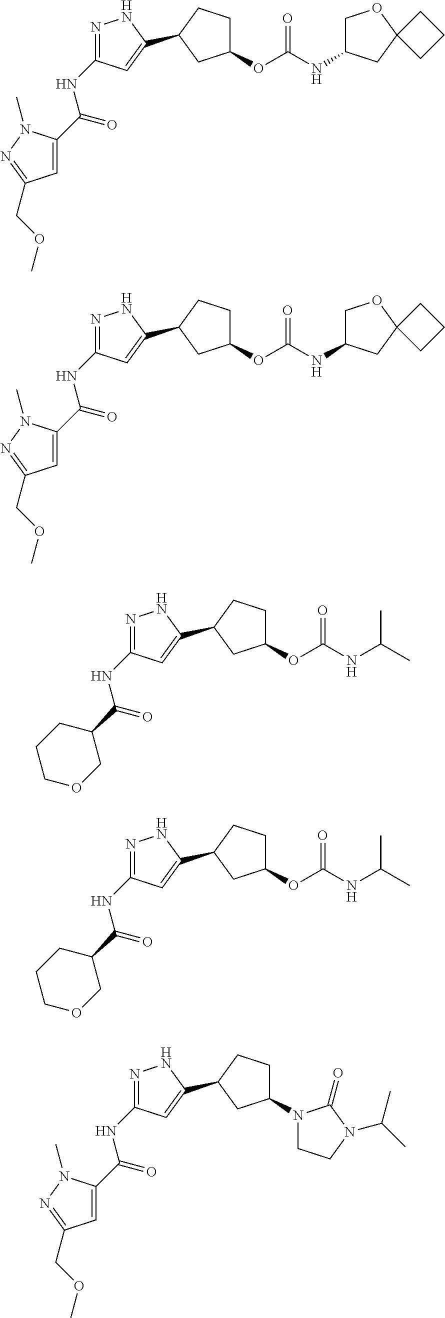 [SMILES: COCc1cc(C(=O)Nc2cc([C@H]3CC[C@@H](OC(=O)N[C@@H]4COC5(CCC5)C4)C3)[nH]n2)n(C)n1 ; COCc1cc(C(=O)Nc2cc([C@H]3CC[C@@H](OC(=O)N[C@H]4COC5(CCC5)C4)C3)[nH]n2)n(C)n1 ; CC(C)NC(=O)O[C@@H]1CC[C@H](c2cc(NC(=O)[C@@H]3CCCOC3)n[nH]2)C1 ; CC(C)NC(=O)O[C@@H]1CC[C@H](c2cc(NC(=O)[C@@H]3CCCOC3)n[nH]2)C1 ; COCc1cc(C(=O)Nc2cc([C@H]3CC[C@@H](N4CCN(C(C)C)C4=O)C3)[nH]n2)n(C)n1]

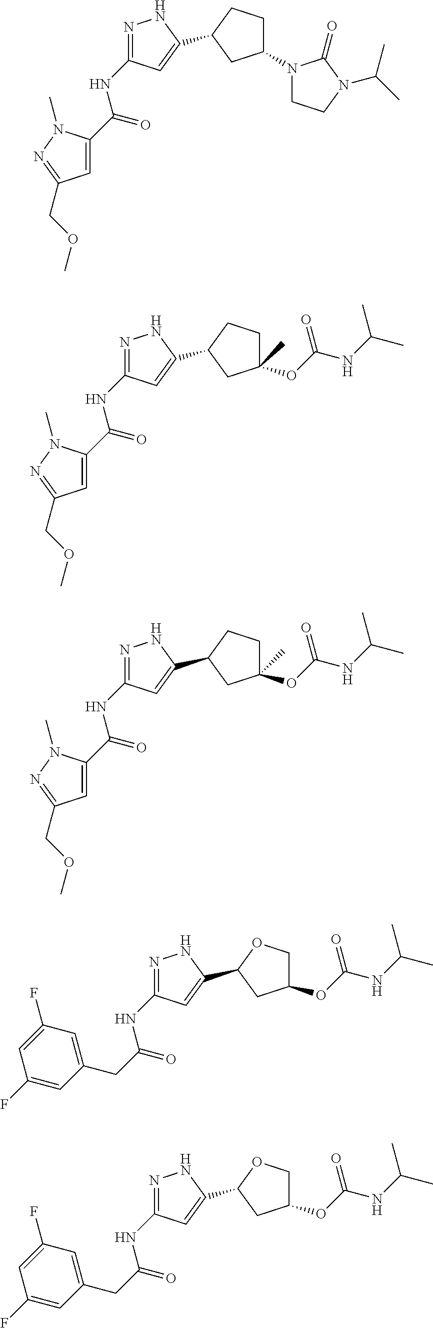 [SMILES: COCc1cc(C(=O)Nc2cc([C@@H]3CC[C@H](N4CCN(C(C)C)C4=O)C3)[nH]n2)n(C)n1 ; COCc1cc(C(=O)Nc2cc([C@@H]3CC[C@](C)(OC(=O)NC(C)C)C3)[nH]n2)n(C)n1 ; COCc1cc(C(=O)Nc2cc([C@H]3CC[C@@](C)(OC(=O)NC(C)C)C3)[nH]n2)n(C)n1 ; CC(C)NC(=O)O[C@@H]1CO[C@H](c2cc(NC(=O)Cc3cc(F)cc(F)c3)n[nH]2)C1 ; CC(C)NC(=O)O[C@H]1CO[C@@H](c2cc(NC(=O)Cc3cc(F)cc(F)c3)n[nH]2)C1]

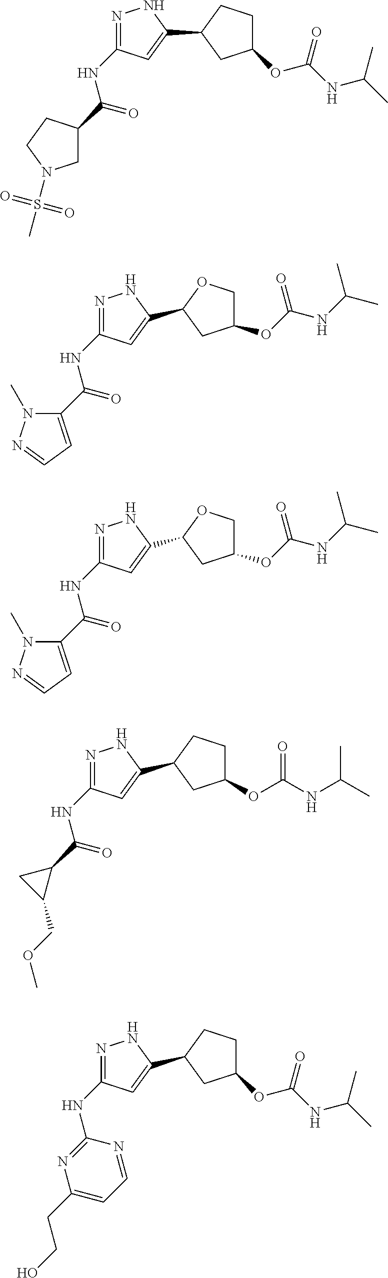

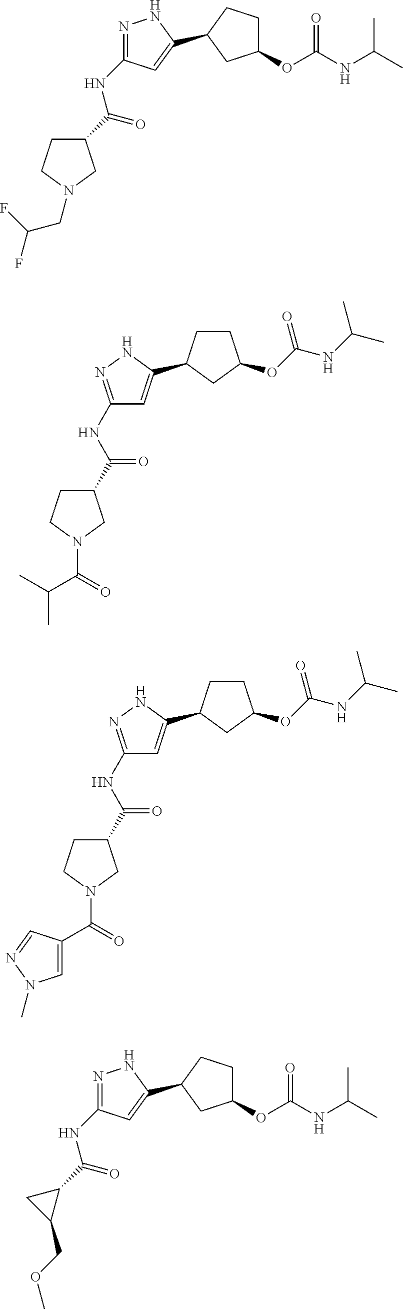

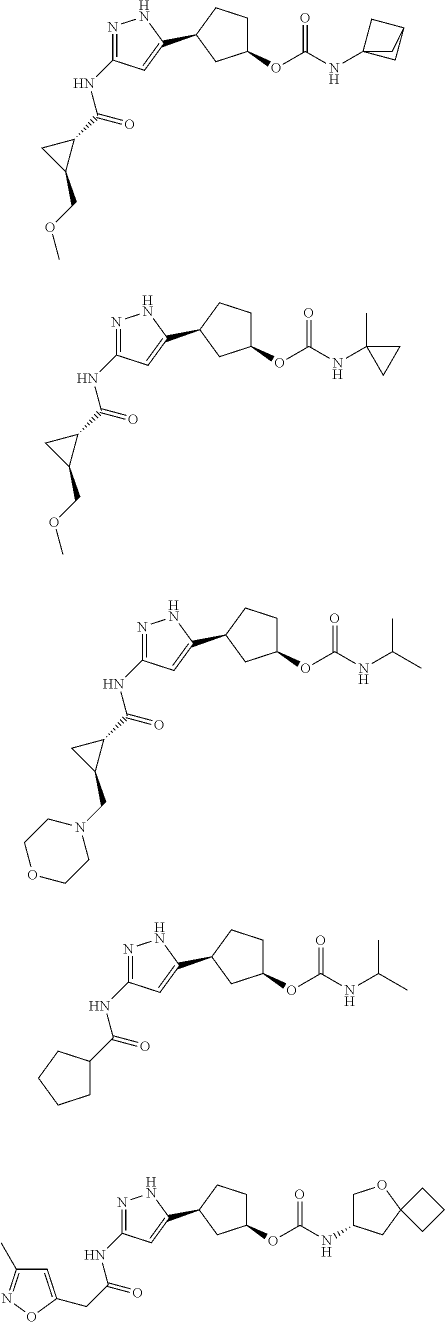

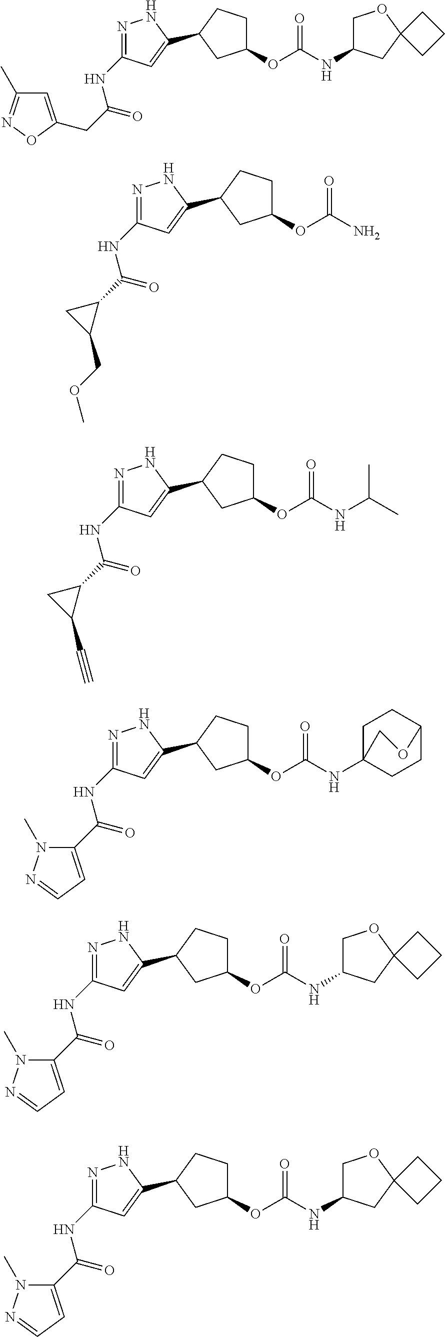

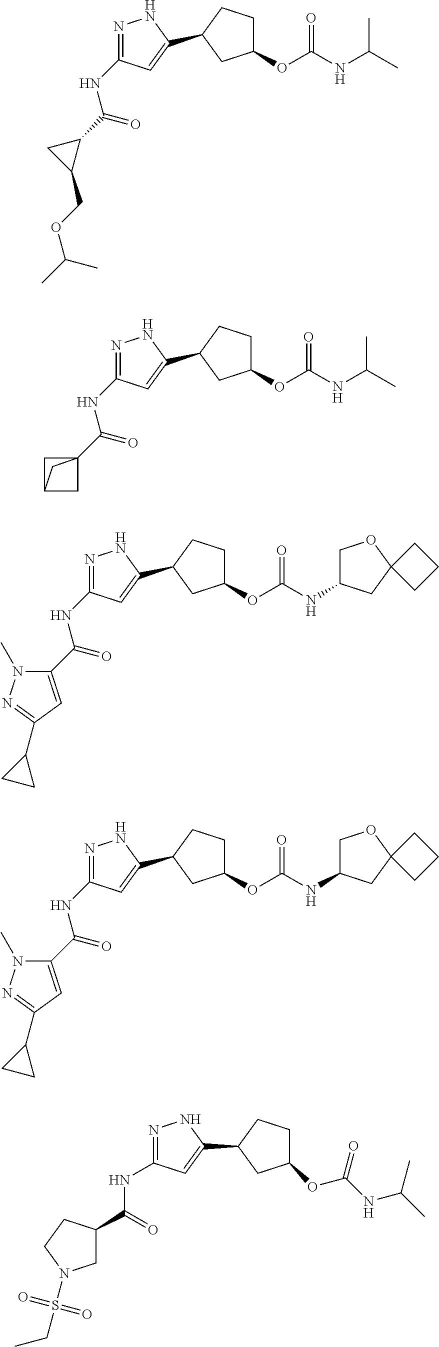

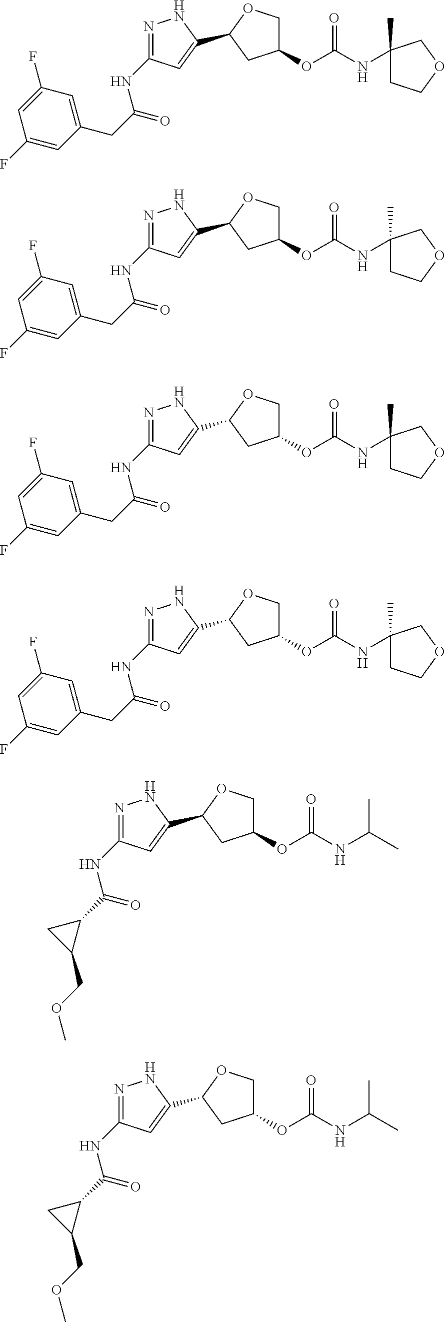 [SMILES: C[C@]1(NC(=O)O[C@@H]2CO[C@H](c3cc(NC(=O)Cc4cc(F)cc(F)c4)n[nH]3)C2)CCOC1 ; C[C@@]1(NC(=O)O[C@@H]2CO[C@H](c3cc(NC(=O)Cc4cc(F)cc(F)c4)n[nH]3)C2)CCOC1 ; C[C@]1(NC(=O)O[C@H]2CO[C@@H](c3cc(NC(=O)Cc4cc(F)cc(F)c4)n[nH]3)C2)CCOC1 ; C[C@@]1(NC(=O)O[C@H]2CO[C@@H](c3cc(NC(=O)Cc4cc(F)cc(F)c4)n[nH]3)C2)CCOC1 ; COC[C@H]1C[C@@H]1C(=O)Nc1cc([C@@H]2C[C@H](OC(=O)NC(C)C)CO2)[nH]n1 ; COC[C@H]1C[C@@H]1C(=O)Nc1cc([C@H]2C[C@@H](OC(=O)NC(C)C)CO2)[nH]n1]

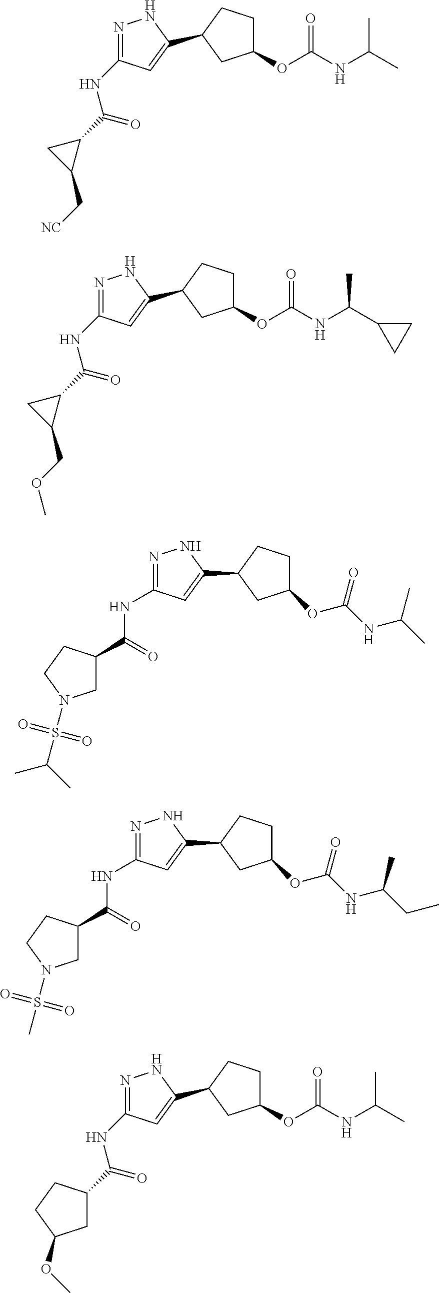

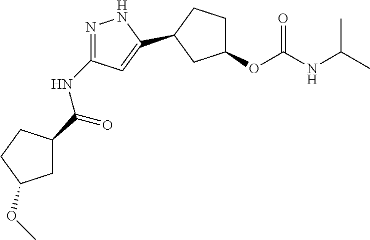 [SMILES: CO[C@@H]1CC[C@@H](C(=O)Nc2cc([C@H]3CC[C@@H](OC(=O)NC(C)C)C3)[nH]n2)C1]

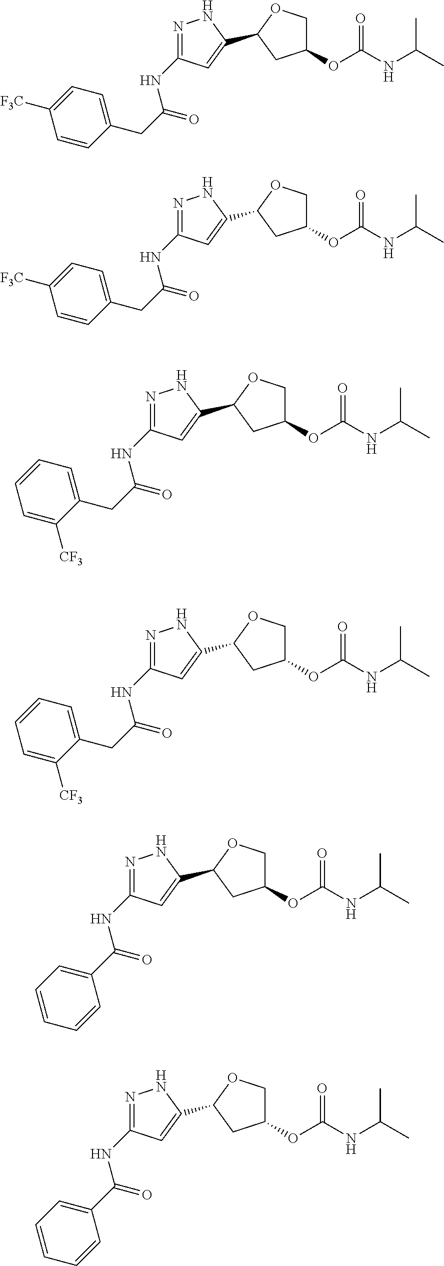 [SMILES: CC(C)NC(=O)O[C@@H]1CO[C@H](c2cc(NC(=O)Cc3ccc(C(F)(F)F)cc3)n[nH]2)C1 ; CC(C)NC(=O)O[C@H]1CO[C@@H](c2cc(NC(=O)Cc3ccc(C(F)(F)F)cc3)n[nH]2)C1 ; CC(C)NC(=O)O[C@@H]1CO[C@H](c2cc(NC(=O)Cc3ccccc3C(F)(F)F)n[nH]2)C1 ; CC(C)NC(=O)O[C@H]1CO[C@@H](c2cc(NC(=O)Cc3ccccc3C(F)(F)F)n[nH]2)C1 ; CC(C)NC(=O)O[C@@H]1CO[C@H](c2cc(NC(=O)c3ccccc3)n[nH]2)C1 ; CC(C)NC(=O)O[C@H]1CO[C@@H](c2cc(NC(=O)c3ccccc3)n[nH]2)C1]

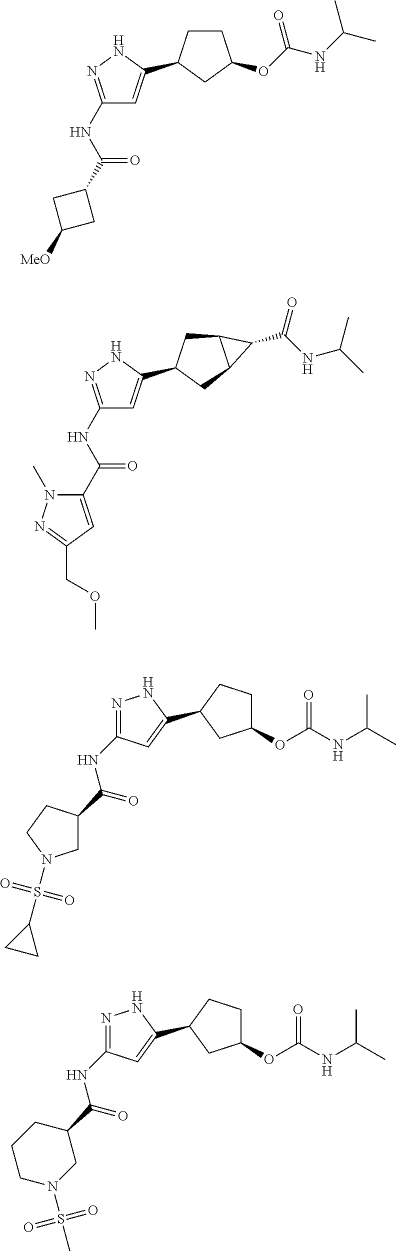

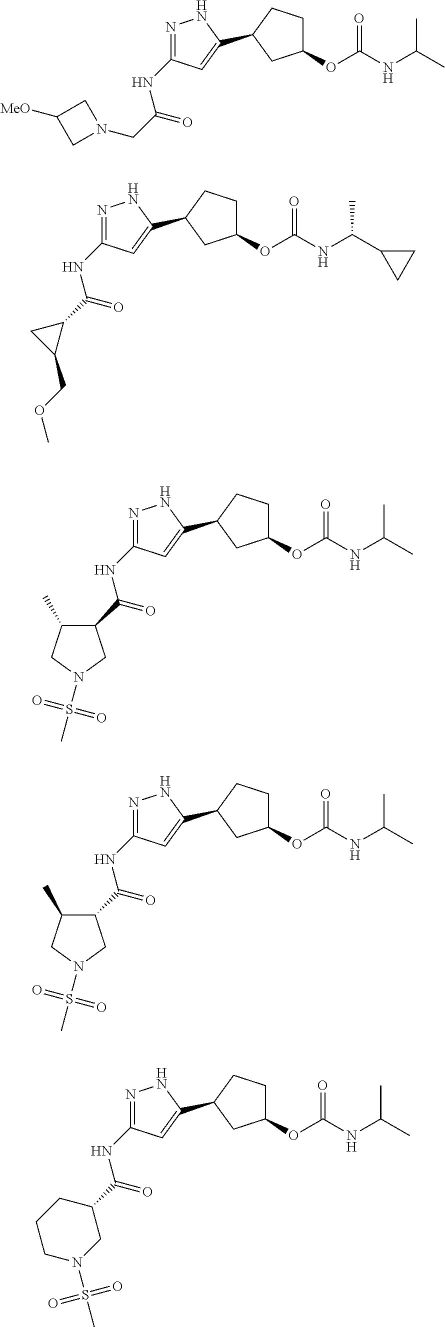 [SMILES: COC1CN(CC(=O)Nc2cc([C@H]3CC[C@@H](OC(=O)NC(C)C)C3)[nH]n2)C1 ; COC[C@H]1C[C@@H]1C(=O)Nc1cc([C@H]2CC[C@@H](OC(=O)N[C@H](C)C3CC3)C2)[nH]n1 ; CC(C)NC(=O)O[C@@H]1CC[C@H](c2cc(NC(=O)[C@H]3CN(S(C)(=O)=O)C[C@@H]3C)n[nH]2)C1 ; CC(C)NC(=O)O[C@@H]1CC[C@H](c2cc(NC(=O)[C@@H]3CN(S(C)(=O)=O)C[C@H]3C)n[nH]2)C1 ; CC(C)NC(=O)O[C@@H]1CC[C@H](c2cc(NC(=O)[C@H]3CCCN(S(C)(=O)=O)C3)n[nH]2)C1]

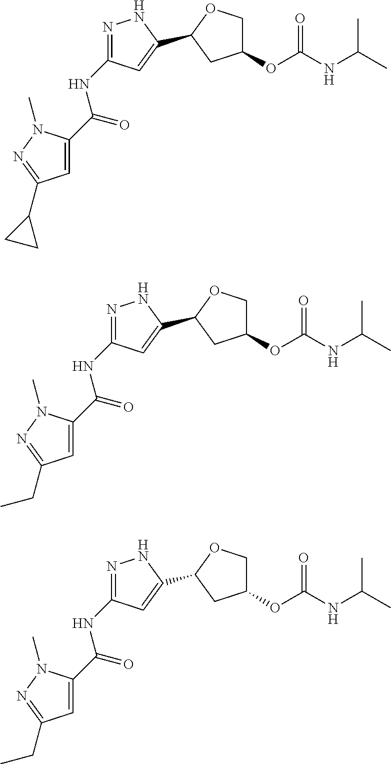

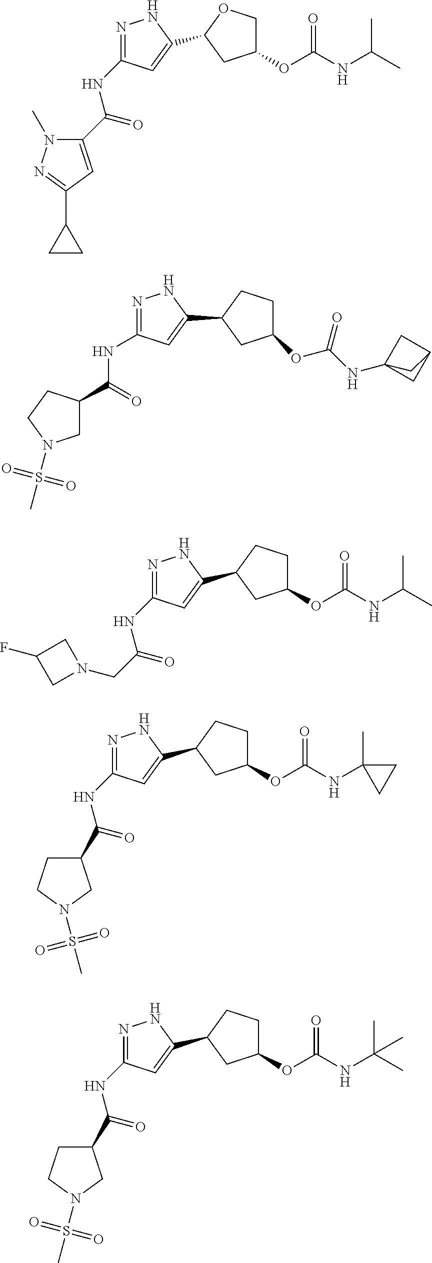 [SMILES: CC(C)NC(=O)O[C@H]1CO[C@@H](c2cc(NC(=O)c3cc(C4CC4)nn3C)n[nH]2)C1 ; CS(=O)(=O)N1CC[C@@H](C(=O)Nc2cc([C@H]3CC[C@@H](OC(=O)NC45CC(C4)C5)C3)[nH]n2)C1 ; CC(C)NC(=O)O[C@@H]1CC[C@H](c2cc(NC(=O)CN3CC(F)C3)n[nH]2)C1 ; CC1(NC(=O)O[C@@H]2CC[C@H](c3cc(NC(=O)[C@@H]4CCN(S(C)(=O)=O)C4)n[nH]3)C2)CC1 ; CC(C)(C)NC(=O)O[C@@H]1CC[C@H](c2cc(NC(=O)[C@@H]3CCN(S(C)(=O)=O)C3)n[nH]2)C1]

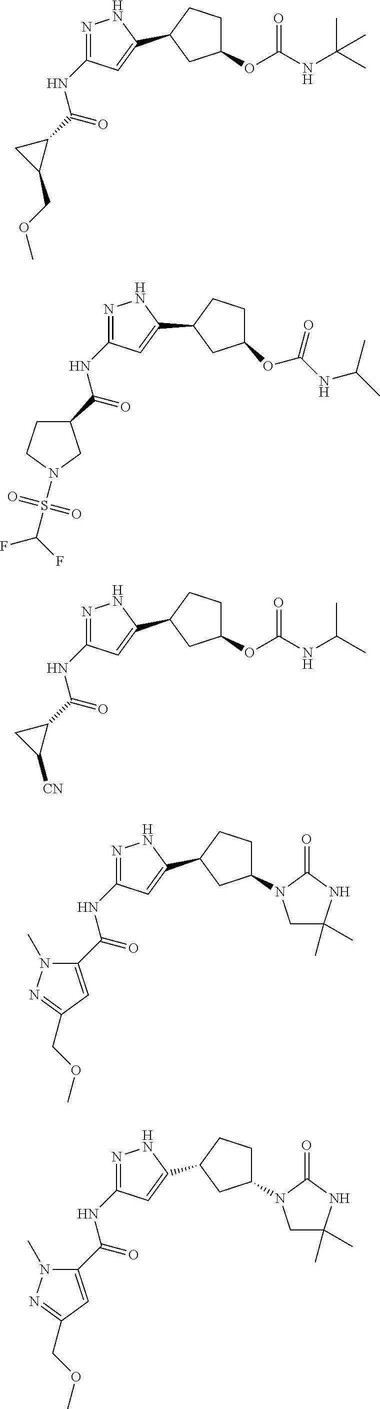

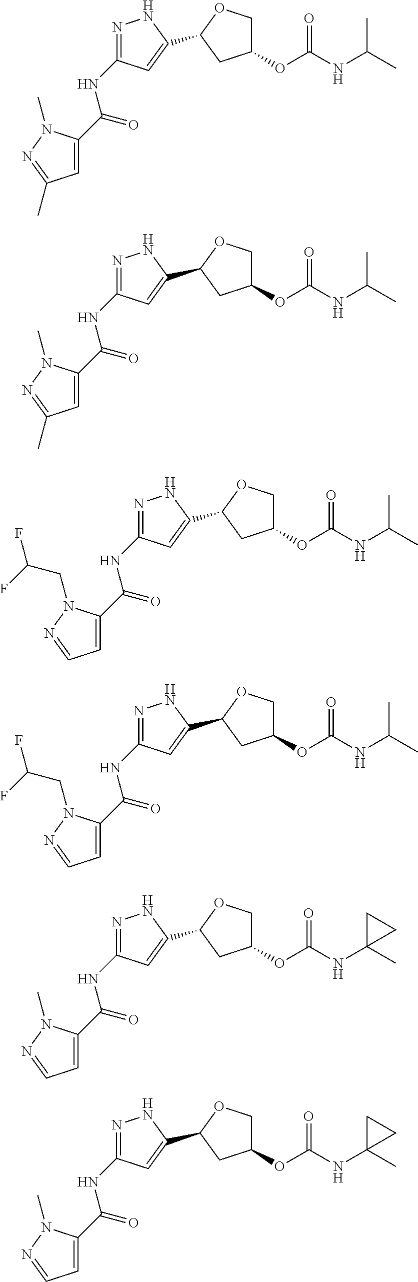 [SMILES: Cc1cc(C(=O)Nc2cc([C@H]3C[C@@H](OC(=O)NC(C)C)CO3)[nH]n2)n(C)n1 ; Cc1cc(C(=O)Nc2cc([C@@H]3C[C@H](OC(=O)NC(C)C)CO3)[nH]n2)n(C)n1 ; CC(C)NC(=O)O[C@H]1CO[C@@H](c2cc(NC(=O)c3ccnn3CC(F)F)n[nH]2)C1 ; CC(C)NC(=O)O[C@@H]1CO[C@H](c2cc(NC(=O)c3ccnn3CC(F)F)n[nH]2)C1 ; Cn1nccc1C(=O)Nc1cc([C@H]2C[C@@H](OC(=O)NC3(C)CC3)CO2)[nH]n1 ; Cn1nccc1C(=O)Nc1cc([C@@H]2C[C@H](OC(=O)NC3(C)CC3)CO2)[nH]n1]

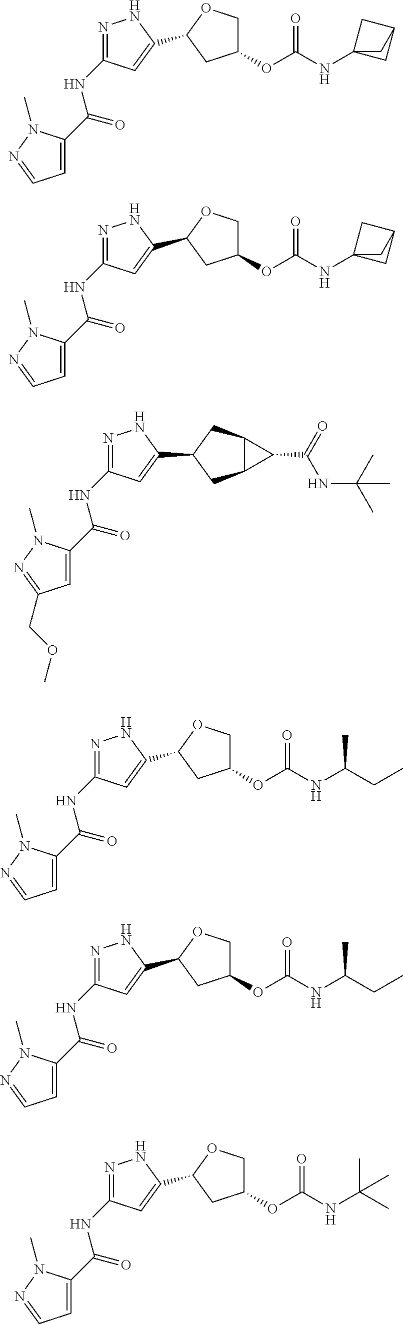 [SMILES: Cn1nccc1C(=O)Nc1cc([C@H]2C[C@@H](OC(=O)NC34CC(C3)C4)CO2)[nH]n1 ; Cn1nccc1C(=O)Nc1cc([C@@H]2C[C@H](OC(=O)NC34CC(C3)C4)CO2)[nH]n1 ; COCc1cc(C(=O)Nc2cc([C@H]3C[C@@H]4[C@H](C3)[C@H]4C(=O)NC(C)(C)C)[nH]n2)n(C)n1 ; CC[C@H](C)NC(=O)O[C@H]1CO[C@@H](c2cc(NC(=O)c3ccnn3C)n[nH]2)C1 ; CC[C@H](C)NC(=O)O[C@@H]1CO[C@H](c2cc(NC(=O)c3ccnn3C)n[nH]2)C1 ; Cn1nccc1C(=O)Nc1cc([C@H]2C[C@@H](OC(=O)NC(C)(C)C)CO2)[nH]n1]

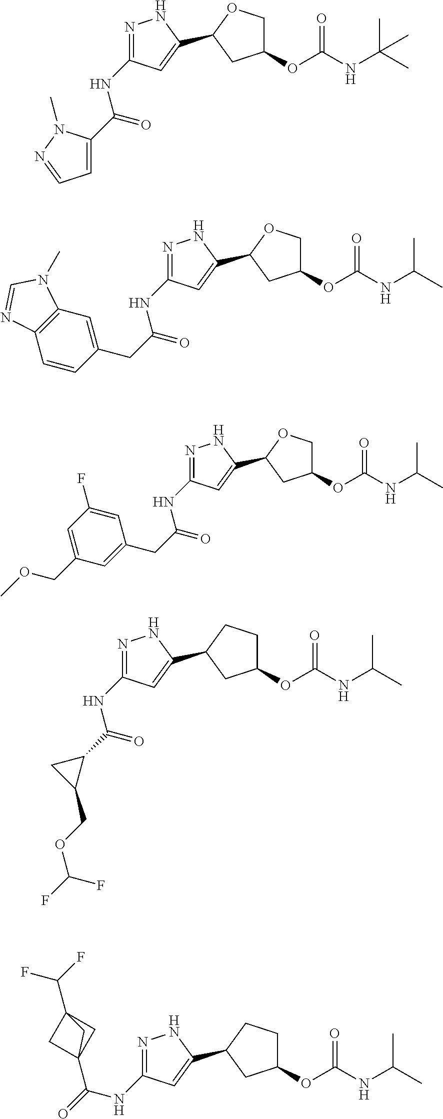

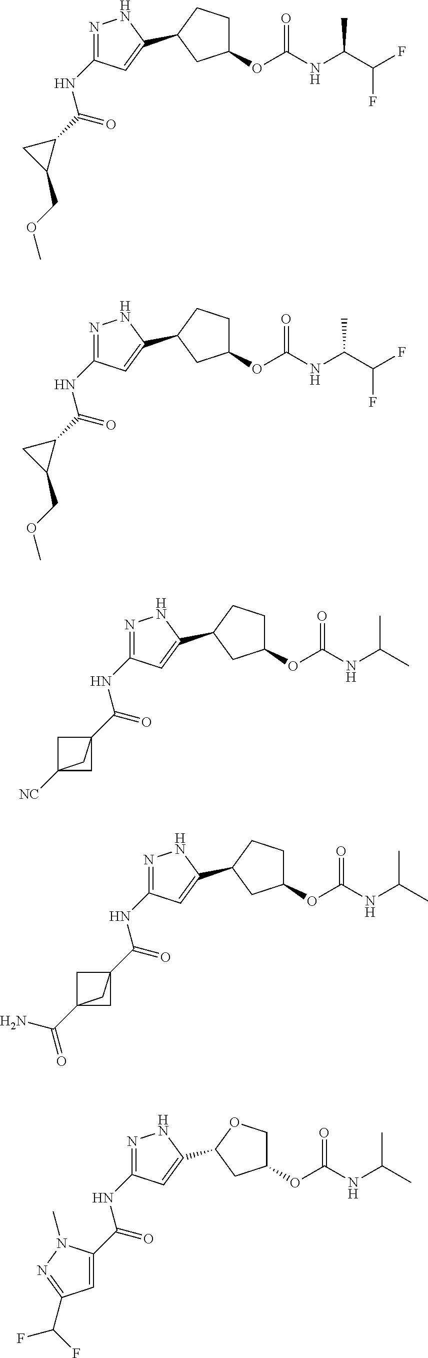 [SMILES: COC[C@H]1C[C@@H]1C(=O)Nc1cc([C@H]2CC[C@@H](OC(=O)N[C@@H](C)C(F)F)C2)[nH]n1 ; COC[C@H]1C[C@@H]1C(=O)Nc1cc([C@H]2CC[C@@H](OC(=O)N[C@H](C)C(F)F)C2)[nH]n1 ; CC(C)NC(=O)O[C@@H]1CC[C@H](c2cc(NC(=O)C34CC(C#N)(C3)C4)n[nH]2)C1 ; CC(C)NC(=O)O[C@@H]1CC[C@H](c2cc(NC(=O)C34CC(C(N)=O)(C3)C4)n[nH]2)C1 ; CC(C)NC(=O)O[C@H]1CO[C@@H](c2cc(NC(=O)c3cc(C(F)F)nn3C)n[nH]2)C1]

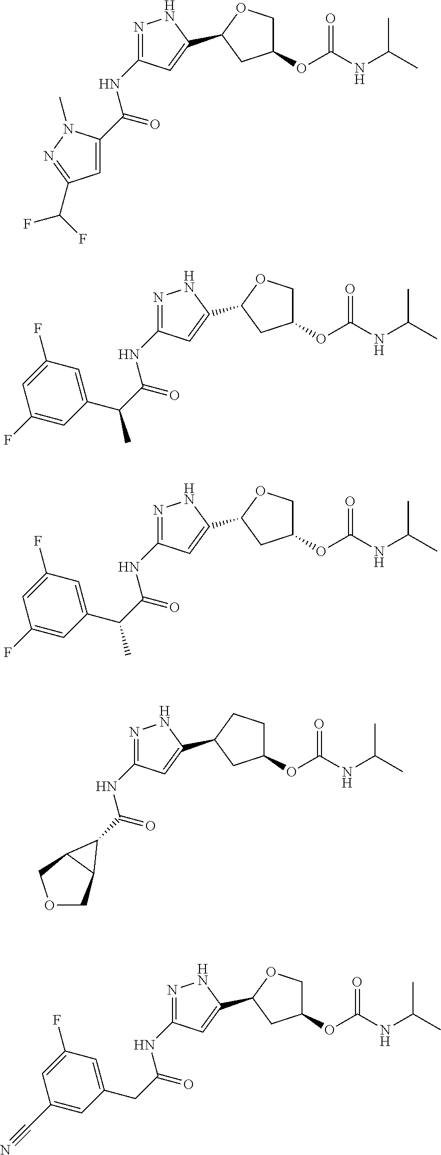 [SMILES: CC(C)NC(=O)O[C@@H]1CO[C@H](c2cc(NC(=O)c3cc(C(F)F)nn3C)n[nH]2)C1 ; CC(C)NC(=O)O[C@H]1CO[C@@H](c2cc(NC(=O)[C@@H](C)c3cc(F)cc(F)c3)n[nH]2)C1 ; CC(C)NC(=O)O[C@H]1CO[C@@H](c2cc(NC(=O)[C@H](C)c3cc(F)cc(F)c3)n[nH]2)C1 ; CC(C)NC(=O)O[C@@H]1CC[C@H](c2cc(NC(=O)[C@H]3[C@@H]4COC[C@@H]43)n[nH]2)C1 ; CC(C)NC(=O)O[C@@H]1CO[C@H](c2cc(NC(=O)Cc3cc(F)cc(C#N)c3)n[nH]2)C1]

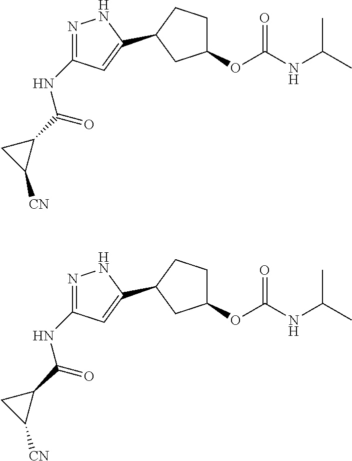 [SMILES: CC(C)NC(=O)O[C@@H]1CC[C@H](c2cc(NC(=O)[C@H]3C[C@@H]3C#N)n[nH]2)C1 ; CC(C)NC(=O)O[C@@H]1CC[C@H](c2cc(NC(=O)[C@@H]3C[C@H]3C#N)n[nH]2)C1]

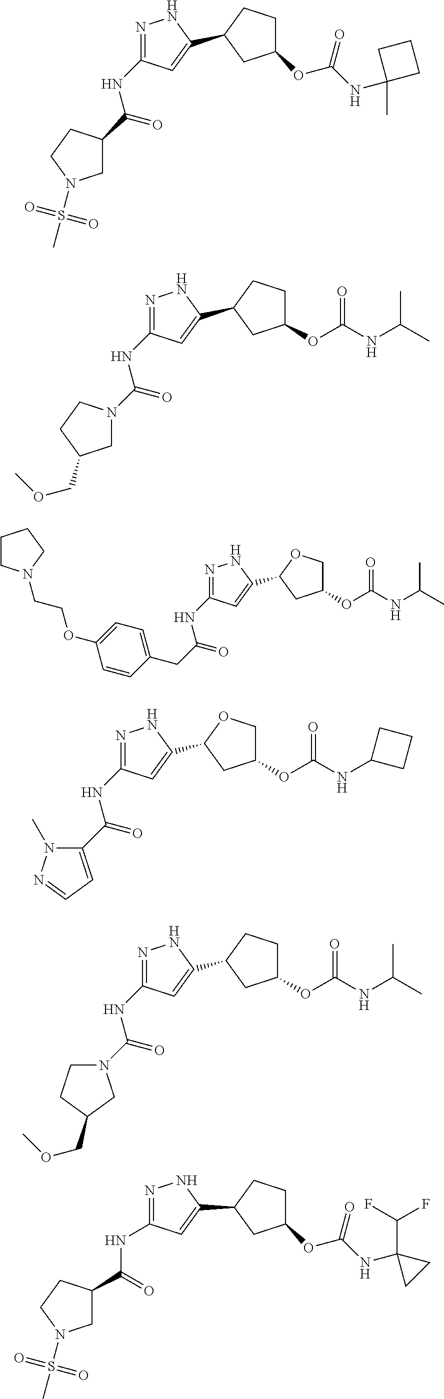

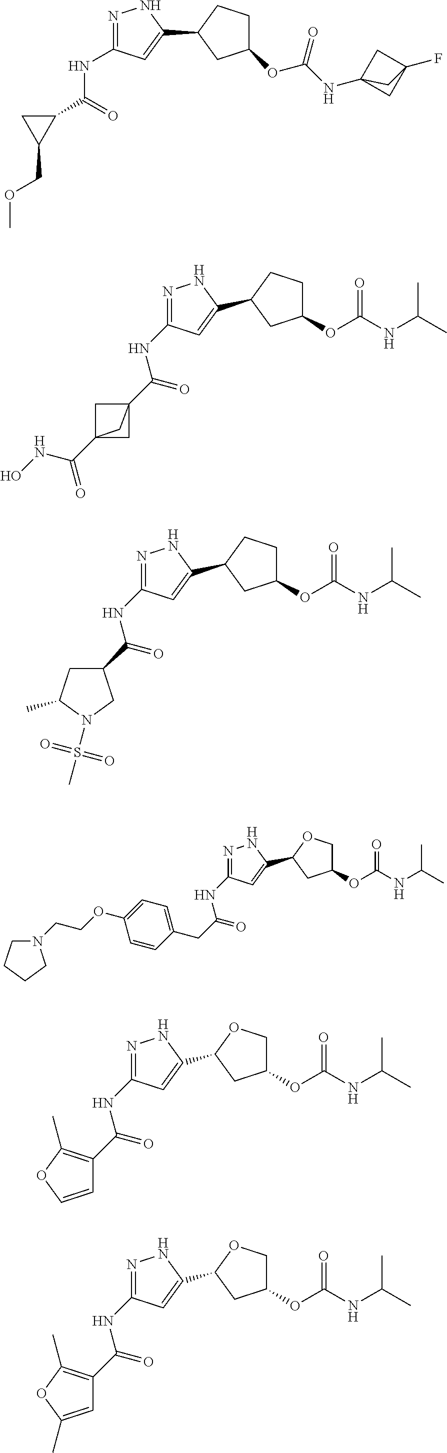

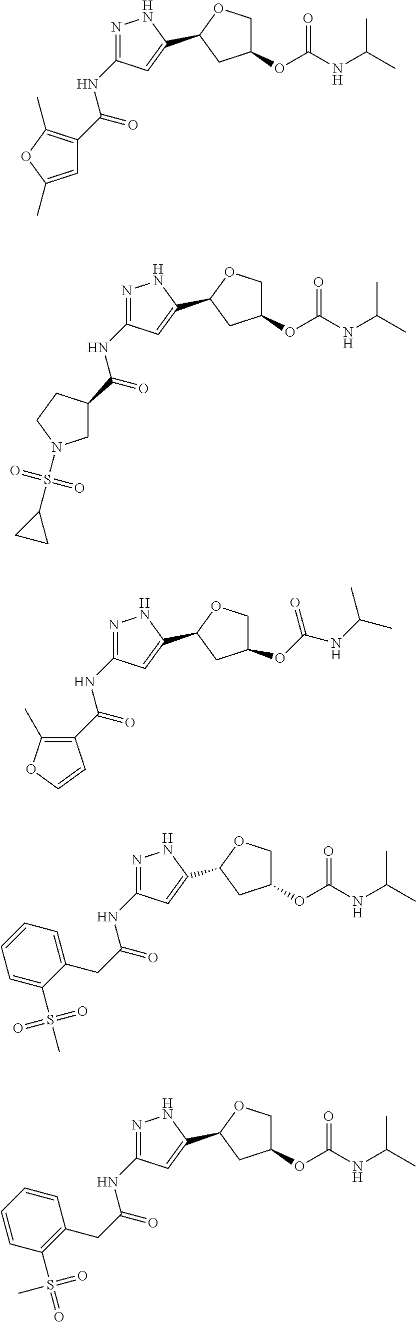

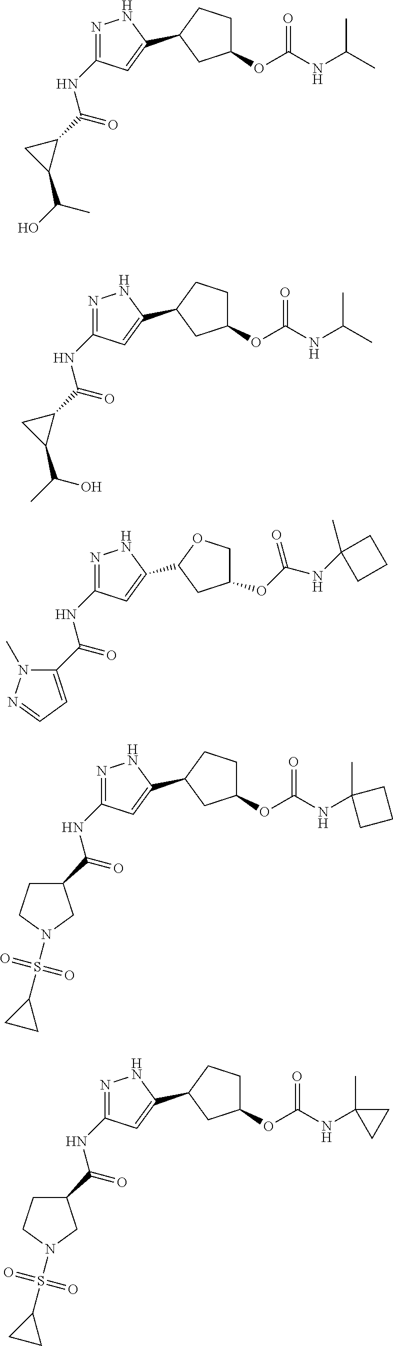

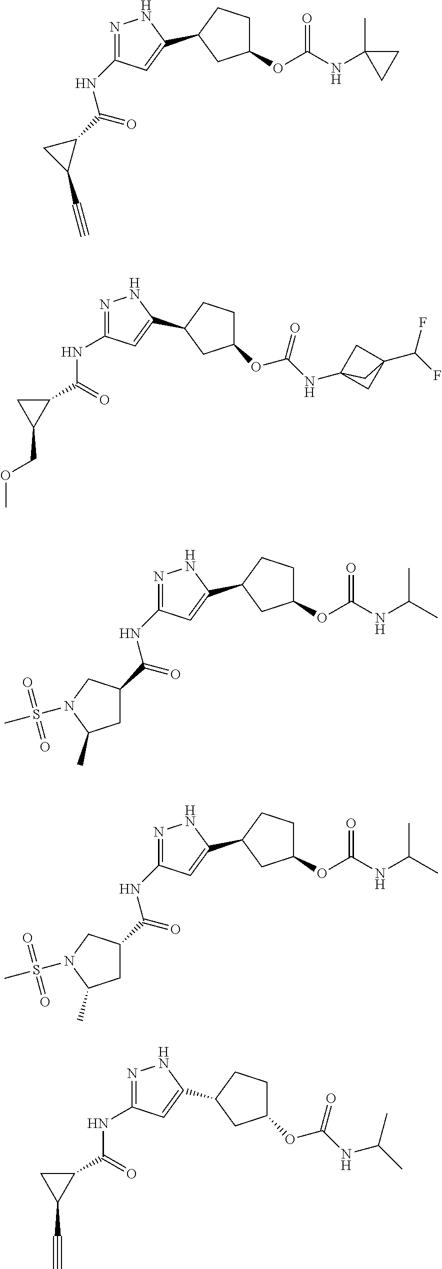

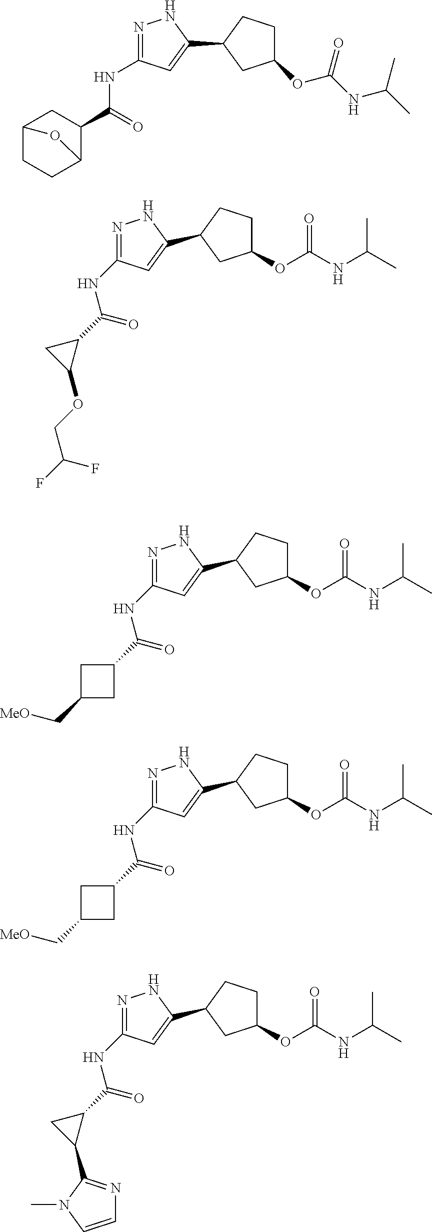

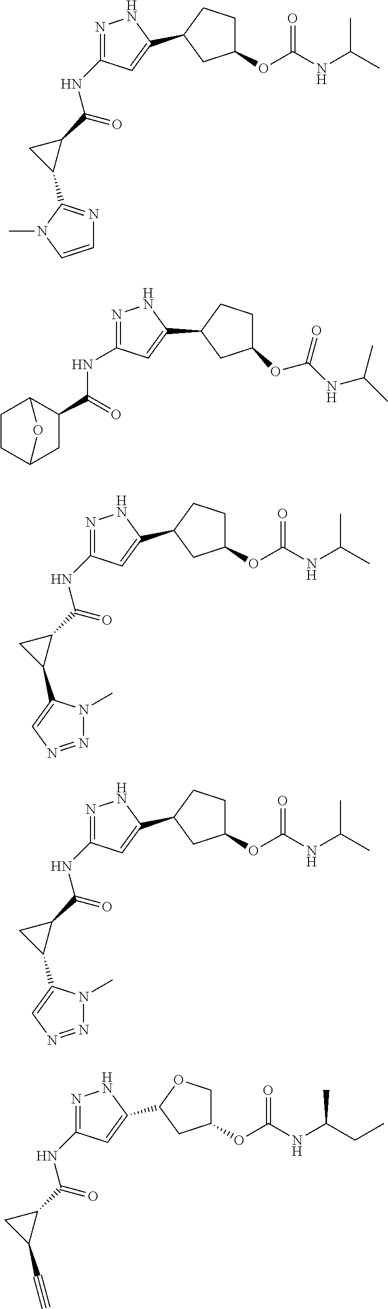

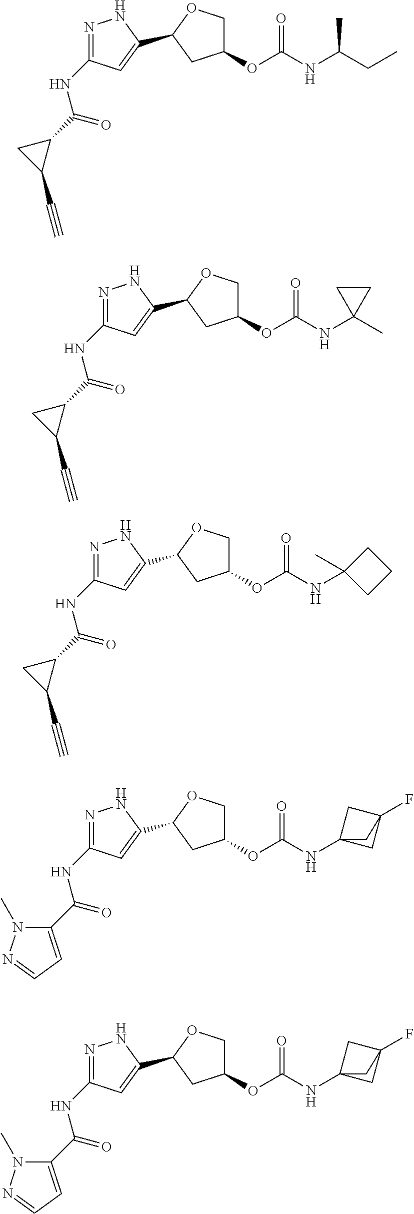

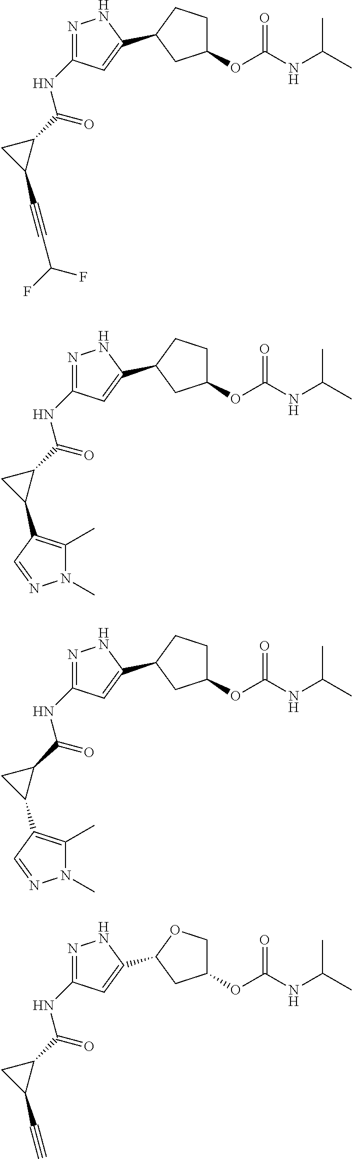 [SMILES: CC(C)NC(=O)O[C@@H]1CC[C@H](c2cc(NC(=O)[C@H]3C[C@@H]3C#CC(F)F)n[nH]2)C1 ; Cc1c([C@H]2C[C@@H]2C(=O)Nc2cc([C@H]3CC[C@@H](OC(=O)NC(C)C)C3)[nH]n2)cnn1C ; Cc1c([C@@H]2C[C@H]2C(=O)Nc2cc([C@H]3CC[C@@H](OC(=O)NC(C)C)C3)[nH]n2)cnn1C ; C#C[C@H]1C[C@@H]1C(=O)Nc1cc([C@H]2C[C@@H](OC(=O)NC(C)C)CO2)[nH]n1]

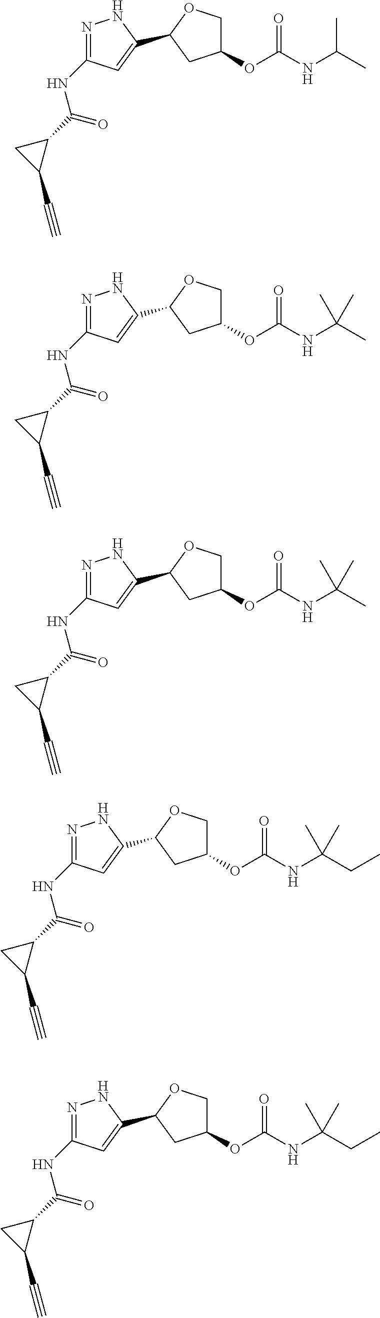 [SMILES: C#C[C@H]1C[C@@H]1C(=O)Nc1cc([C@@H]2C[C@H](OC(=O)NC(C)C)CO2)[nH]n1 ; C#C[C@H]1C[C@@H]1C(=O)Nc1cc([C@H]2C[C@@H](OC(=O)NC(C)(C)C)CO2)[nH]n1 ; C#C[C@H]1C[C@@H]1C(=O)Nc1cc([C@@H]2C[C@H](OC(=O)NC(C)(C)C)CO2)[nH]n1 ; C#C[C@H]1C[C@@H]1C(=O)Nc1cc([C@H]2C[C@@H](OC(=O)NC(C)(C)CC)CO2)[nH]n1 ; C#C[C@H]1C[C@@H]1C(=O)Nc1cc([C@@H]2C[C@H](OC(=O)NC(C)(C)CC)CO2)[nH]n1]

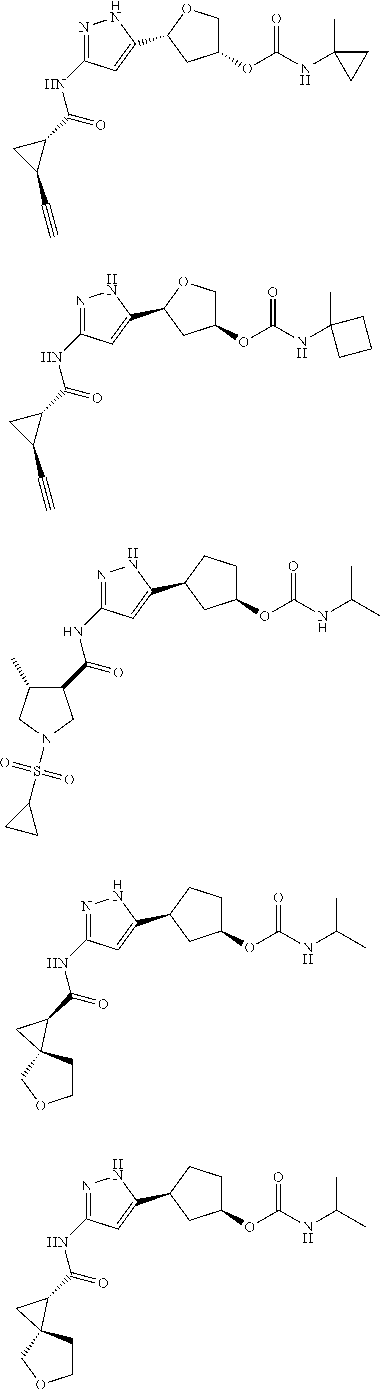

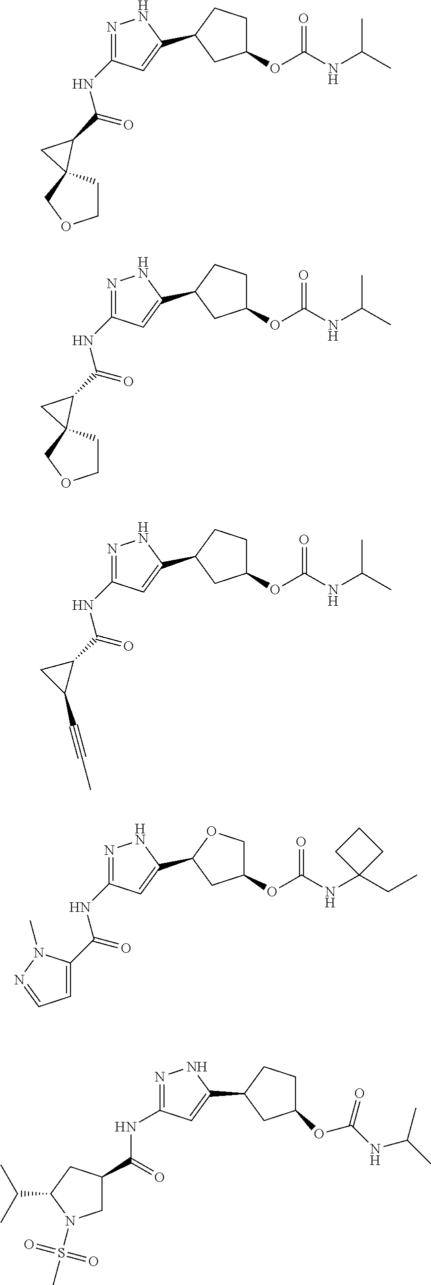

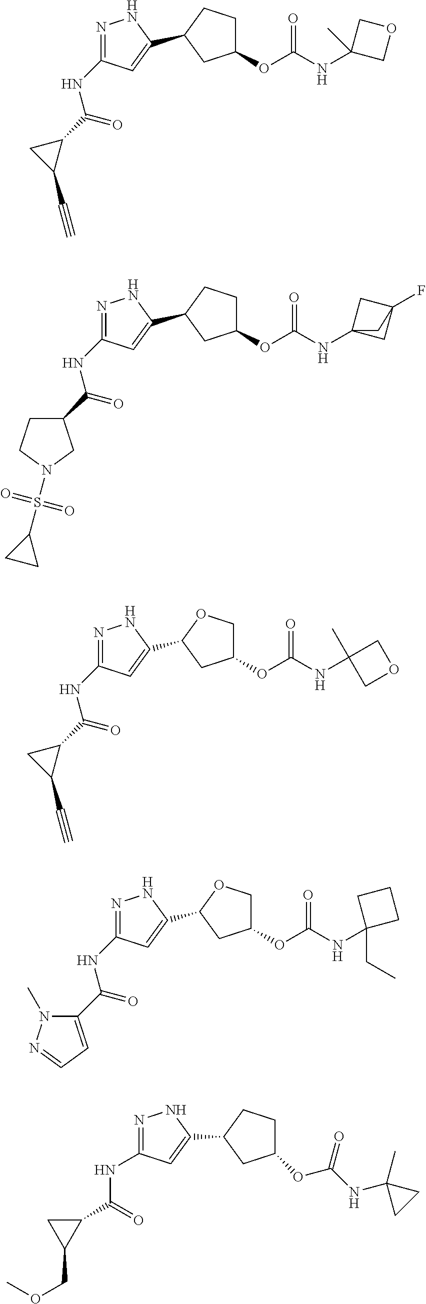 [SMILES: C#C[C@H]1C[C@@H]1C(=O)Nc1cc([C@H]2CC[C@@H](OC(=O)NC3(C)COC3)C2)[nH]n1 ; O=C(NC12CC(F)(C1)C2)O[C@@H]1CC[C@H](c2cc(NC(=O)[C@@H]3CCN(S(=O)(=O)C4CC4)C3)n[nH]2)C1 ; C#C[C@H]1C[C@@H]1C(=O)Nc1cc([C@H]2C[C@@H](OC(=O)NC3(C)COC3)CO2)[nH]n1 ; CCC1(NC(=O)O[C@H]2CO[C@@H](c3cc(NC(=O)c4ccnn4C)n[nH]3)C2)CCC1 ; COC[C@H]1C[C@@H]1C(=O)Nc1cc([C@@H]2CC[C@H](OC(=O)NC3(C)CC3)C2)[nH]n1]

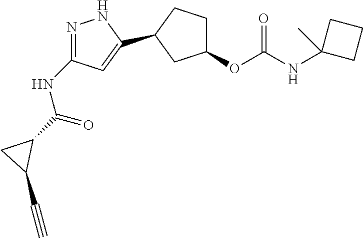 [SMILES: C#C[C@H]1C[C@@H]1C(=O)Nc1cc([C@H]2CC[C@@H](OC(=O)NC3(C)CCC3)C2)[nH]n1]

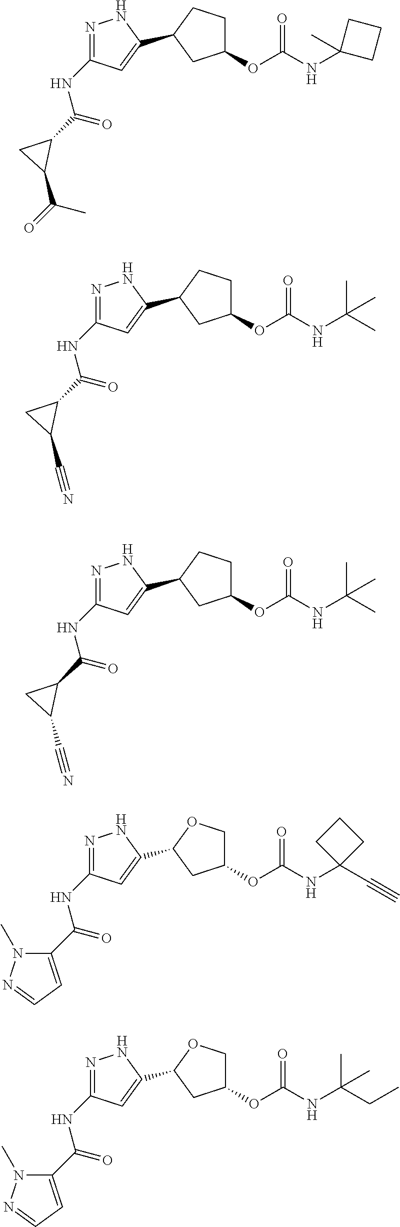

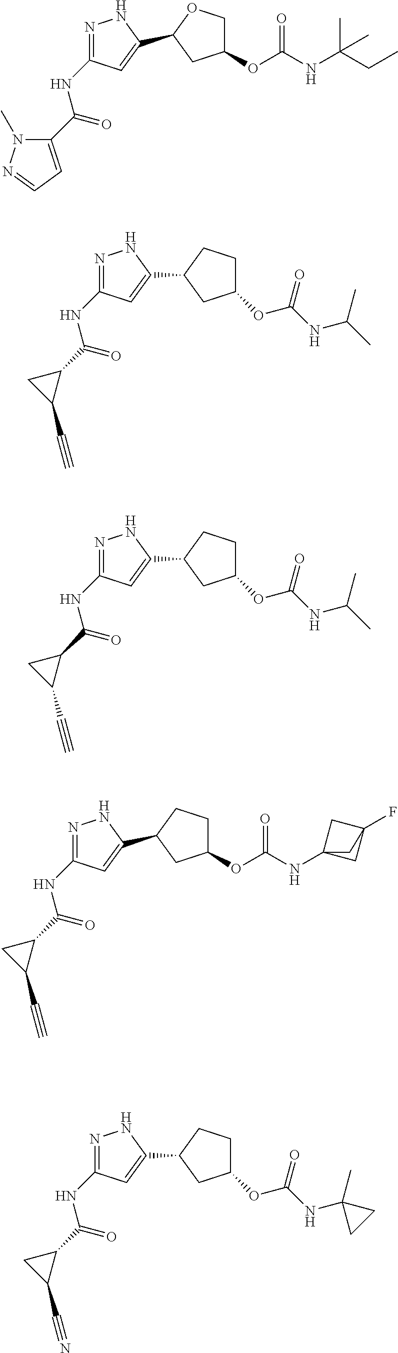 [SMILES: CCC(C)(C)NC(=O)O[C@@H]1CO[C@H](c2cc(NC(=O)c3ccnn3C)n[nH]2)C1 ; C#C[C@H]1C[C@@H]1C(=O)Nc1cc([C@@H]2CC[C@H](OC(=O)NC(C)C)C2)[nH]n1 ; C#C[C@@H]1C[C@H]1C(=O)Nc1cc([C@@H]2CC[C@H](OC(=O)NC(C)C)C2)[nH]n1 ; C#C[C@H]1C[C@@H]1C(=O)Nc1cc([C@H]2CC[C@@H](OC(=O)NC34CC(F)(C3)C4)C2)[nH]n1 ; CC1(NC(=O)O[C@H]2CC[C@@H](c3cc(NC(=O)[C@H]4C[C@@H]4C#N)n[nH]3)C2)CC1]

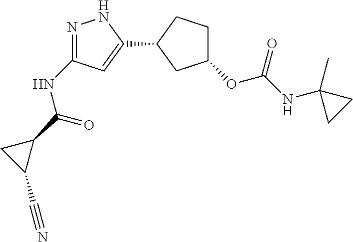 [SMILES: CC1(NC(=O)O[C@H]2CC[C@@H](c3cc(NC(=O)[C@@H]4C[C@H]4C#N)n[nH]3)C2)CC1]

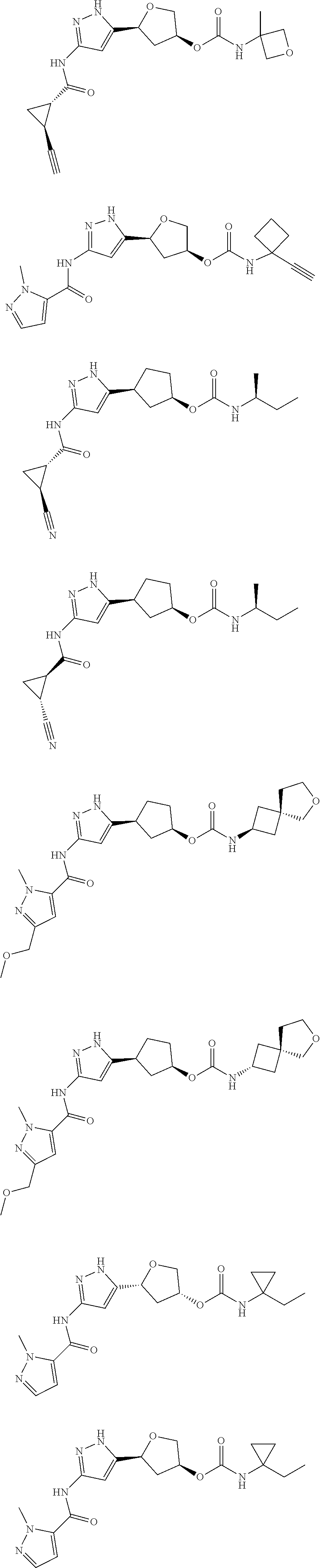

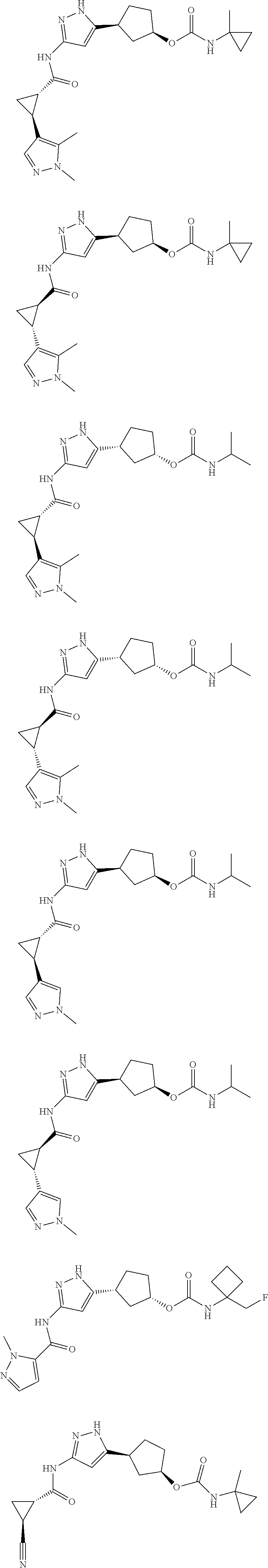

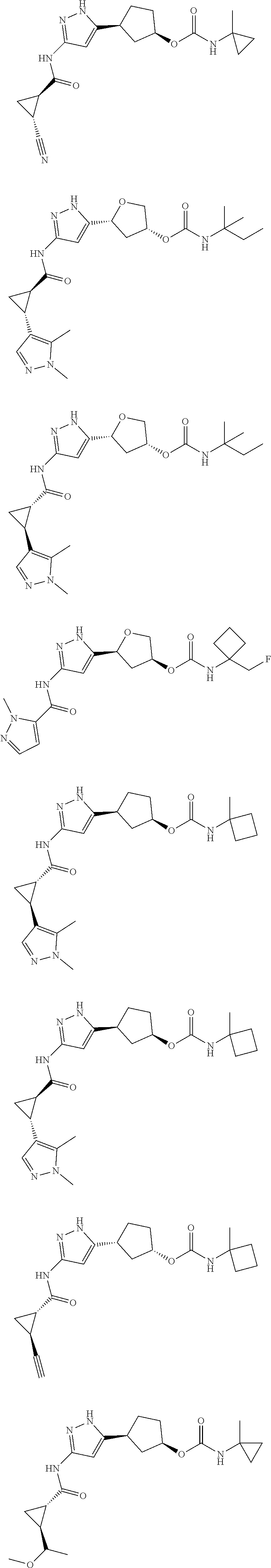

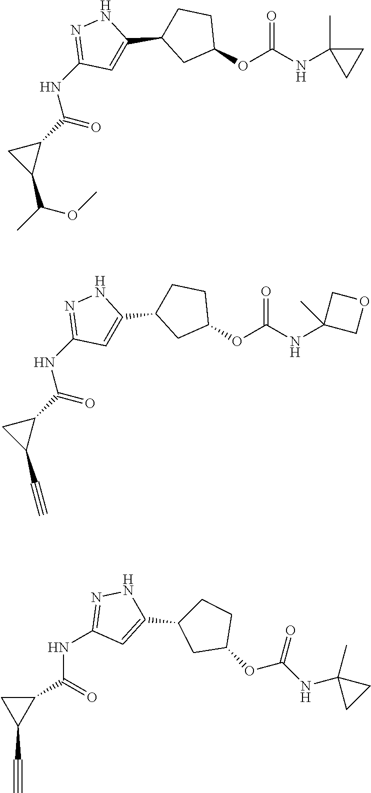 [SMILES: COC(C)[C@H]1C[C@@H]1C(=O)Nc1cc([C@H]2CC[C@@H](OC(=O)NC3(C)CC3)C2)[nH]n1 ; C#C[C@H]1C[C@@H]1C(=O)Nc1cc([C@@H]2CC[C@H](OC(=O)NC3(C)COC3)C2)[nH]n1 ; C#C[C@H]1C[C@@H]1C(=O)Nc1cc([C@@H]2CC[C@H](OC(=O)NC3(C)CC3)C2)[nH]n1]

27. A pharmaceutical composition comprising the compound, or an optical isomer, prodrug, or pharmaceutically acceptable salt thereof according to any one of claims 1 to 26, and a pharmaceutically acceptable excipient or carrier.

28. Use of the compound, or an optical isomer, prodrug, or pharmaceutically acceptable salt thereof according to any one of claims 1 to 26 in preparation of a CDK2 inhibitor.

29. Use of the compound, or an optical isomer, prodrug, or pharmaceutically acceptable salt thereof according to any one of claims 1 to 26 in preparation of a drug for treating an abnormal cell growth disease.

30. A method of treating an abnormal cell growth disease, comprising administering to a subject in need thereof an effective amount of the compound, or an optical isomer, prodrug, or pharmaceutically acceptable salt thereof according to any one of claims 1 to 26 or the pharmaceutical composition according to claim 27.

31. The use according to claim 29 or the method according to claim 30, characterized in that, the abnormal cell growth disease is cancer.

32. The use or method according to claim 31, characterized in that, the cancer is selected from breast cancer, ovarian cancer, bladder cancer, uterine cancer, prostate cancer, lung cancer, esophageal cancer, head and neck cancer, colorectal cancer, kidney Cancer, liver cancer, pancreatic cancer, stomach cancer or thyroid cancer.

33. The use or method according to claim 32, characterized in that, the lung cancer is selected from non-small cell lung cancer, small cell lung cancer, squamous cell carcinoma or adenocarcinoma.
